(19) United States
(12) Patent Application Publication
(10) Pub. No.: US 2022/0195462 A1
(43) Pub. Date: Jun. 23, 2022

(54) FULLY-HUMAN POST-TRANSLATIONALLY MODIFIED ANTIBODY THERAPEUTICS

(71) Applicant: REGENXBIO INC., Rockville, MD (US)

(72) Inventors: Olivier Danos, Princeton, NJ (US); Zuchun Wu, North Potomac, MD (US); Ye Liu, Clarksville, MD (US); Sherri Van Everen, Menlo Park, CA (US); Franz Gerner, Myersville, MD (US); Joseph Bruder, Gaithersburg, MD (US); Chunping Qiao, Rockville, MD (US); Devin McDougald, Boston, MA (US); Xu Wang, Falls Church, VA (US); Justin Glenn, Rockville, MD (US)

(21) Appl. No.: 17/605,486

(22) PCT Filed: Apr. 24, 2020
(86) PCT No.: PCT/US20/29802

§ 371 Date: Oct. 21, 2021

Related U.S. Application Data
(60) Provisional application No. 62/838,165, filed on Apr. 24, 2019.
(60) Provisional application No. 62/967,472, filed on Jan. 29, 2020.

(51) Int. Cl. C12N 15/86; A61P 17/00; C07K 16/40

(52) U.S. Cl. CPC C12N 15/86; A61P 17/00; A61K 2039/505; C07K 2317/21; C12N 2750/14141; C07K 16/40

(57) ABSTRACT

Provided are methods and compositions for the delivery of fully human post-translationally modified therapeutic monoclonal antibodies and antigen-binding fragments thereof. The fully human post-translationally modified therapeutic monoclonal antibodies may be delivered by gene therapy methods, e.g., as a recombinant adeno-associated vims (rAAV) vector to the appropriate tissue. Methods of manufacture of the AAV vectors, pharmaceutical compositions and methods of treatment are also provided. In addition, provided are methods of producing therapeutic antibodies that are “biobetters” as fully human post-translationally modified. These fully human post-translationally modified therapeutic antibodies may be administered to a subject in need of treatment with the therapeutic antibody.

### 0. SEQUENCE LISTING

The instant application contains a Sequence Listing which has been submitted electronically in ASCII format and is hereby incorporated by reference in its entirety. Said ASCII copy, created on Apr. 24, 2020, is named 38013_0001P1_SL.txt and is 690,185 bytes in size.

### 1. INTRODUCTION

Compositions and methods are described for the delivery of a fully human post-translationally modified (HuPTM) therapeutic monoclonal antibody (“mAb”) or the HuPTM antigen-binding fragment of a therapeutic mAb—e.g., a fully human-glycosylated (HuGly) Fab of the therapeutic mAb—to a human subject diagnosed with a disease or condition indicated for treatment with the therapeutic mAb.

### 2. BACKGROUND OF THE INVENTION

Therapeutic mAbs have been shown to be effective in treating a number of diseases and conditions. However, because these agents are effective for only a short period of time, repeated injections for long durations are often required, thereby creating considerable treatment burden for patients.

### 3. SUMMARY OF THE INVENTION

Compositions and methods are described for the delivery of a HuPTM mAb or a HuPTM antigen-binding fragment of a therapeutic mAb (for example, a fully human-glycosylated Fab (HuGlyFab) of a therapeutic mAb) to a patient (human subject) diagnosed with a disease or condition indicated for treatment with the therapeutic mAb. Such antigen-binding fragments of therapeutic mAbs include a Fab, F(ab′)2, or scFv (single-chain variable fragment) (collectively referred to herein as “antigen-binding fragment”). “HuPTM Fab” as used herein may include other antigen binding fragments of a mAb. In an alternative embodiment, full-length mAbs can be used. Delivery may be advantageously accomplished via gene therapy—e.g., by administering a viral vector or other DNA expression construct encoding a therapeutic mAb or its antigen-binding fragment (or a hyperglycosylated derivative of either) to a patient (human subject) diagnosed with a condition indicated for treatment with the therapeutic mAb—to create a permanent depot in a tissue or organ of the patient that continuously supplies the HuPTM mAb or antigen-binding fragment of the therapeutic mAb, e.g., a human-glycosylated transgene product, to a target tissue where the mAb or antigen-binding fragment there of exerts its therapeutic effect.

The HuPTM mAb or HuPTM antigen-binding fragment encoded by the transgene can include, but is not limited to, a full-length or an antigen-binding fragment of a therapeutic antibody that binds to:
Nervous system targets, including Amyloid beta (Aβ or Abeta) peptides derived from the amyloid precursor protein (APP), including but not limited solanezumab, GSK933776, and lecanemab (see FIGS. 2A-C), indicated for treating Alzheimer's disease; sortilin, including but not limited to AL-001 (see FIG. 3), for treating frontotemporal dementia (FTD); Tau protein implicated in tauopathies, including Alzheimer's disease, progressive supranuclear palsy, FTD, chronic traumatic encephalopathy, Pick's Complex, primary age-related tauopothy, including but not limited to ABBV-8E12, UCB-0107, and NI-105 (BIIB076) (see FIGS. 4A-C) for treating tauopathies; SEMA4D, including but not limited to VX15/2503 (see FIG. 5), for treating Huntington's disease and juvenile Huntington's disease; alpha-synuclein, including but not limited to prasinezumab, NI-202 (BIIB054) and MED-1341 (see FIGS. 6A-C), for treating Parkinson's disease and synucleinopathies; superoxide dismutase-1 (SOD-1), including but not limited NI-204 (see FIGS. 7A and B), for treating ALS and Alzheimer's disease; and CGRP receptor, including but not limited to eptinezumab, fremanezumab, and galcanezumab (see FIGS. 8A-C), for treating migraines and cluster headaches;
Ocular Anti-Angiogenic Targets, including but not limited to VEGF (vascular endothelial growth factor), including but not limited to sevacizumab (see FIG. 9A), for treating retinal disorders including diabetic retinopathy (DR), myopic choroidal neovascularization (mCNV), age-related macular degeneration (AMD), and macular edema; erythropoietin receptor, including but not limited to LKA-651 (see FIGS. 9B and C), indicated for treatment of retinal diseases such as retinal vein occlusion (RVO), wet AMD, and macular edema; amyloid beta (Aβ or Abeta) peptides derived from the amyloid precursor protein (APP), including but not limited to solanezumab, GSK933776, or lecanemab (see FIGS. 2A-C), for treating dry AMD; activin receptor like kinase 1 (ALK1), including but not limited to ascrinvacumab (see FIG. 10A), indicated for treating neovascular age-related macular degeneration; complement component 5 (C5), including but not limited to tesidolumab and ravulizumab (see FIGS. 10B and 10D), indicated for treating dry AMD and non-infectious uveitis, endoglin (END or CD105), including but not limited to carotuximab (see FIG. 10C), indicated for treating wet AMD and other retinal disorders caused by increased vascularization; complement component 1Q (C1Q), including but not limited to ANX-007 (see FIG. 11), indicated for treating glaucoma; and plasma protein targets, such as human complement proteins including but not limited to plasma kallikrein (pKal), including but not limited to lanadelumab (see FIG. 19) for treating diabetic retinopathy and diabetic macular edema;
Complement component 5, including but not limited to ravulizumab, indicated for treating myasthenia gravis (see FIG. 10D);
TNF-alpha, including but not limited, to adalimumab (HUMIRA®), infliximab (REMICADE®), and golimumab, indicated for treating non-infectious uveitis (see FIGS. 12A-C);
Repulsive guidance molecule-A, including but not limited to elezanumab (see FIG. 13), for treating multiple sclerosis;
Transthyretin (TTR), including but not limited to NI-301 and PRX-004 (see FIGS. 14A and B), indicated for treating amyloidosis;
Connective tissue growth factor (CTGF), including but not limited to pamrevlumab (see FIG. 15), indicated for treating fibrotic diseases (e.g. diabetic nephropathy, liver fibrosis, idiopathic pulmonary fibrosis);
Neuromyelitis optica (NMO)/Non-Infectious Uveitis targets, including the TNF-alpha targeted antibodies above, and interleukin 6 (IL6)- and interleukin 6 receptor (IL6R)-targeted antibodies, including but not limited to satralizumab, sarilumab, siltuximab, clazakizumab, sirukumab, olokizumab, gerilimzumab, and tocilizumab (see FIGS. 16A-H), indicated for treating NMO, DR, DME, and non-infectious uveitis; and CD19, including but not limited to inebilizumab (see FIG. 16I), indicated for treating NMO;
Immune response targets, including interleukin 6 (IL6)- and interleukin 6 receptor (IL6R)-targeted antibodies, including but not limited to satralizumab, sarilumab, siltuximab, clazakizumab, sirukumab, olokizumab, gerilimzumab, and tocilizumab (see FIGS. 16A-H), indicated for treating adverse immune responses, such as cytokine release syndrome, such as associated with bacterial or viral infections, and to be administered with immune effecting agents, such as CAR-T and other cell based therapies, and immunooncology agents to counteract, reduce or ameliorate detrimental immune responses associated with such therapies;
Integrin beta 7, including but not limited to etrolizumab (see FIG. 17), indicated for treating ulcerative colitis and Crohn's disease;
Sclerostin, including but not limited to romosozumab (EVENITY®) (see FIG. 18), indicated for treating osteoporosis and abnormal bone loss or weakness;
Plasma Protein targets, such as human complement proteins including but not limited to plasma kallikrein, including but not limited to lanadelumab (see FIG. 19) for treating hereditary angioedema and ocular indications, including diabetic retinopathy and diabetic macular edema; and
Anti-IL and IL Receptor and other targets for autoimmune, respiratory and allergic diseases, such as interleukin 5 (IL5), including but not limited to benralizumab (see FIG. 29A); interleukin 5 receptor (IL5R), including but not limited to reslizumab (see FIG. 29B); interleukin 13 (IL13), including but not limited to tralokinumab (see FIG. 29C); interleukin 31 receptor alpha (IL-31RA), including but not limited to nemolizumab (see FIG. 29D); immunoglobin E (IgE), including but not limited to omalizumab (see FIG. 29E); and thymic stromal lymphopoietin (TSLP), including but not limited to tezepelumab (see FIG. 29F). or antigen-binding fragments.

The recombinant vector used for delivering the transgene includes non-replicating recombinant adeno-associated virus vectors (“rAAV”). However, other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements.

Gene therapy constructs are designed such that both the heavy and light chains are expressed. The coding sequences for the heavy and light chains can be engineered in a single construct in which the heavy and light chains are separated by a cleavable linker or IRES so that separate heavy and light chain polypeptides are expressed. In certain embodiments, the coding sequences encode for a Fab or F(ab′)₂ or an scFv. In certain embodiments the full length heavy and light chains of the antibody are expressed. In other embodiments, the constructs express an scFv in which the heavy and light chain variable domains are connected via a flexible, non-cleavable linker. In certain embodiments, the construct expresses, from the N-terminus, NH₂—V_{L}-linker-V_{H}—COOH or NH₂—V_{H}-linker-V_{L}—COOH.

Therapeutic antibodies delivered by gene therapy have several advantages over injected or infused therapeutic antibodies that dissipate over time resulting in peak and trough levels. Sustained expression of the transgene product antibody, as opposed to injecting an antibody repeatedly, allows for a more consistent level of antibody to be present at the site of action, and is less risky and more convenient for patients, since fewer injections need to be made. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

Pharmaceutical compositions suitable for administration to human subjects comprise a suspension of the recombinant vector in a formulation buffer comprising a physiologically compatible aqueous buffer, a surfactant and optional excipients.

The invention is based, in part, on the following principles:
(i) The mAb therapeutics currently on the market are of the immunoglobulin G (IgG) isotypes, such as IgG1, IgG2, and IgG4, which in general have pharmacokinetic (PK) characteristics, such as slow clearance, long half-life, and limited tissue distribution. After intravenous administration, typical mAb serum PK profiles are biphasic with a rapid distribution phase and a slower elimination phase; thus, repeat administration is required to maintain doses required to treat chronic conditions. Moreover, the distribution of mAbs is generally limited to the vascular and interstitial spaces due to their large size and hydrophilicity. The extent of mAb partitioning from circulation into most tissues generally ranges from about 5-15%, except for brain where it is much lower. (See, e.g., Kamath, 2016, Drug Discovery Today: Technologies 21-22: 75-83, which is incorporated by reference herein in its entirety). Continuous production of HuPTMmAbs or HuPTM Fabs in situ avoids repeat administrations and allows the use of Fabs, which would otherwise have too short a systemic half-life to achieve efficacy; and the methods of administration described allow direct access to target tissues, such as the brain, where the delivery of higher doses to such tissues can be achieved.
(ii) The Fab region of a number of therapeutic mAbs possesses glycosylation sites. For example, see FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F which identify consensus and non-consensus asparaginal (“N”) glycosylation sites as well as glutamine (“Q”) residues that are glycosylation sites in the Fab region of certain therapeutic mAbs. (See, e.g., Valliere-Douglass et al., 2009, J. Biol. Chem. 284: 32493-32506, and Valliere-Douglass et al., 2010, J. Biol. Chem. 285: 16012-16022, each of which is incorporated by reference in its entirety for the identification of N-linked glycosylation sites in antibodies). In addition, O-glycosylation comprises the addition of N-acetyl-galactosamine to serine or threonine residues by the enzyme. It has been demonstrated that amino acid residues present in the hinge region of antibodies can be O-glycosylated. The possibility of O-glycosylation confers another advantage to the therapeutic antibodies provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria are modified to contain specific O-glycosylation machinery. See, e.g., Farid-Moayer et al., 2007, J. Bacteriol. 189:8088-8098.) Moreover, the Fab amino acid sequence may be modified to engineer hyperglycosylated variants (e.g., see amino acid substitutions that can be made to engineer hyperglycosylated Fab regions of therapeutic antibodies shown in FIGS. 20A and 20B; and Courtois et al., 2016, mAbs 8: 99-112 which is incorporated by reference herein in its entirety for it description of derivatives of antibodies that are hyperglycosylated on the Fab domain of the full-length antibody).
(iii) In addition to the glycosylation sites, the Fab regions can contain tyrosine (“Y”) sulfation sites in or near the CDRs; see FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F which identify tyrosine-O-sulfation sites in the Fab region of certain therapeutic mAbs. (See, e.g., Yang et al., 2015, Molecules 20:2138-2164 (particularly at 2154), which is incorporated by reference in its entirety for the analysis of amino acids surrounding tyrosine residues subjected to protein tyrosine sulfation). The “rules” can be summarized as follows: Y residues with E or D within +5 to −5 position of Y, and where position −1 of Y is a neutral or acidic charged amino acid—but not a basic amino acid, e.g., R, K, or H that abolishes sulfation.
(iv) The glycosylation of Fab regions, such as those shown in FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F and Fc regions (see FIG. 22 and Table 7) by human cells will result in the addition of glycans that can improve stability, half-life and reduce unwanted aggregation and/or immunogenicity of the transgene product. (See, e.g., Bovenkamp et al., 2016, J. Immunol. 196: 1435-1441 for a review of the emerging importance of Fab glycosylation; and FIG. 22 which identifies glycans that can be attached to HuGlyFab (adapted from Bondt et al., 2014, Mol & Cell Proteomics 13.1: 3029-2029)). The Fab and Fc portions of antibodies have been shown to have distinct glycosylation patterns, with Fab glycans being high in galactosylation, sialylation, and bisection (e.g., with bisecting GlcNAc) but low in fucosylation with respect to Fc glycans. (E.g., see Bondt et al., 2014, Mol. & Cell. Proteomics 13.11:3029-3039, incorporated by reference herein in its entirety for its disclosure of Fab-associated N-glycans).
(v) Significantly, glycans that are added to HuPTM mAb and HuGlyFab of the invention are highly processed complex-type N-glycans that contain 2,6-sialic acid. Such glycans are not present in (a) therapeutic mAbs produced in E. coli (which are not glycosylated at all); (b) in therapeutic antibodies produced in CHO cells that do not have the 2,6-sialyltransferase required to add 2,6-sialic acid during glycosylation; or (c) in therapeutic antibodies produced in either CHO or murine cell lines that add N-Glycolylneuraminic acid (“Neu5Gc” or “NeuGc”) which is not natural to humans (and potentially immunogenic), instead of N-Acetylneuraminic acid (“Neu5Ac”) the predominant human sialic acid. See, e.g., Dumont et al., 2015, Crit. Rev. Biotechnol. 36(6):1110-1122; Huang et al., 2006, Anal. Biochem. 349:197-207 (NeuGc is the predominant sialic acid in murine cell lines such as SP2/0 and NS0); and Song et al., 2014, Anal. Chem. 86:5661-5666, each of which is incorporated by reference herein in its entirety.
(vi) The human glycosylation pattern of the HuPTM mAb and HuGlyFab of the invention should reduce immunogenicity of the transgene product and improve efficacy. Importantly, when the full-length antibodies and antigen-binding fragments, used in accordance with the methods described herein are expressed in human target cells, the need for in vitro production in prokaryotic host cells (e.g., E. coli) or eukaryotic host cells (e.g., CHO cells or murine NS0 or SP2/0 cells) is circumvented. Instead, as a result of the methods described herein (e.g., use of human target cells to express the antigen-binding fragments), N-glycosylation sites of the full-length antibodies and antigen-binding fragments are advantageously decorated with glycans relevant to and beneficial to treatment of humans. Such an advantage is unattainable when CHO cells, murine cells, or E. coli are utilized in antibody/antigen-binding fragment production, because, e.g., (a) CHO cells lack components needed for addition of certain glycans (e.g., 2,6 sialic acid and bisecting GlcNAc); (b) CHO cells and murine cells (NS0 and SP2/0 cells) add Neu5Gc as sialic acid not typical to humans instead of Neu5Ac; (c) CHO cells can also produce an immunogenic glycan, the α-Gal antigen, which reacts with anti-α-Gal antibodies present in most individuals, which at high concentrations can trigger anaphylaxis (see, e.g., Bosques, 2010, Nat Biotech 28:1153-1156); and (d) E. coli does not naturally contain components needed for N-glycosylation.
(vii) Tyrosine-sulfation of Fab regions, such as those shown in FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F—a robust post-translational process in many human cells—should result in transgene products with increased avidity for their molecular targets. Indeed, tyrosine-sulfation of the Fab of antibodies has been shown to dramatically increase avidity for antigen and activity. (See, e.g., Loos et al., 2015, PNAS 112: 12675-12680, and Choe et al., 2003, Cell 114: 161-170). Such post-translational modifications are not present on therapeutic antibodies made in E. coli (a host that does not possess the enzymes required for tyrosine-sulfation), and at best are under-represented in therapeutic mAbs made in CHO cells. CHO cells are not secretory cells and have a limited capacity for post-translational tyrosine-sulfation. (See, e.g., Mikkelsen & Ezban, 1991, Biochemistry 30: 1533-1537, especially discussion at p. 1537).

For the foregoing reasons, the production of HuPTM mAb or HuPTM Fab should result in a “biobetter” molecule for the treatment of disease accomplished via gene therapy—e.g., by administering a viral vector or other DNA expression construct encoding a full-length HuPTM mAb or HuPTM Fab of a therapeutic mAb to a patient (human subject) diagnosed with a disease indication for that mAb, to create a permanent depot in the subject that continuously supplies the human-glycosylated, sulfated transgene product produced by the subject's transduced cells. The cDNA construct for the HuPTMmAb or HuPTM Fab should include a signal peptide that ensures proper co- and post-translational processing (glycosylation and protein sulfation) by the transduced human cells.

As an alternative, or an additional treatment to gene therapy, the full-length HuTPM mAb or HuPTM Fab can be produced in human cell lines by recombinant DNA technology, and the glycoprotein can be administered to patients.

Combination therapies involving delivery of the full-length HuPTM mAb or HuPTM Fab to the patient accompanied by administration of other available treatments are encompassed by the methods provided herein. The additional treatments may be administered before, concurrently or subsequent to the gene therapy treatment. Such additional treatments can include but are not limited to co-therapy with the therapeutic mAb.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

The inventors have also found that full length antibodies can be expressed from AAV based vectors (see Examples 36 and 37). The nucleotide sequence encoding the heavy and light chains of the full-length antibodies may be codon optimized for expression in human cells and may have reduced numbers of CpG dimers in the sequence. Accordingly, provided are compositions comprising AAV vectors that express a transgene encoding a full-length heavy chain (including an Fc domain) and light chain of a therapeutic antibody. Methods of administration and manufacture are also provided.

### 3.1 Illustrative Embodiments

### Compositions of Matter

1. A pharmaceutical composition for treating Alzheimer's disease (AD), frontotemporal dementia (FD), tauopathies, progressive supranuclear palsy, chronic traumatic encephalopathy, Pick's Complex, and primary age-related tauopathy, Huntington's disease, juvenile Huntington's disease, Parkinson's disease, synucleinopathies, ALS, migraines, or cluster headaches in a human subject in need thereof, comprising an adeno-associated virus (AAV) vector having:
(a) a viral capsid that is at least 95% identical to the amino acid sequence of an AAV8 capsid (SEQ ID NO: 143), AAV9 capsid (SEQ ID NO: 144), AAVrh10 capsid (SEQ ID NO: 145), an AAVrh20 capsid, an AAVrh39 capsid or an AAVcy5 capsid; and
(b) an artificial genome comprising an expression cassette flanked by AAV inverted terminal repeats (ITRs), wherein the expression cassette comprises a transgene encoding a substantially full-length or full-length mAb of an anti-amyloid beta (anti-Aβ), anti-sortilin, anti-Tau protein (anti-Tau), anti-semaphorin 4D (anti-SEMA4D), anti-alpha synuclein (anti-SNCA), anti-superoxide dismutase-1 (anti-SOD1) or anti-calcitonin gene-related peptide receptor (anti-CGRPR) monoclonal antibody (mAb), or an antigen-binding fragment thereof, operably linked to one or more regulatory sequences that control expression of the transgene in human CNS cells, human liver cells and/or human muscle cells;
wherein said AAV vector is formulated for administration to said subject, optionally wherein administration is intrathecal, intravenous, subcutaneous, intranasal, or intramuscular.

2. The pharmaceutical composition of paragraph 1, wherein the anti-Aβ mAb is solanezumab, lecanemab, or GSK933776; the anti-sortilin mAb is AL-001; the anti-Tau mAb is ABBV-8E12, UCB-0107, or NI-105 (BIIB076); the anti-SEMA4D mAb is VX15/2503; the anti-SNCA mAb is prasinezumab, NI-202 (BIIB054), or MED-1341; the anti-SOD1 mAb is NI-2041.10D12 or NI-204.12G7; and the anti-CGRPR mAb is eptinezumab, fremanezumab, or galcanezumab.

3. The pharmaceutical composition of paragraphs 1 or 2, wherein the antigen binding fragment is a Fab, a F(ab′)₂, or a single chain variable domain (scFv).

4. The pharmaceutical composition of any of paragraphs 1 to 3, wherein the full-length mAb or the antigen-binding fragment comprises a heavy chain with an amino acid sequence of SEQ ID NO: 1 and optionally an Fc polypeptide with an amino acid sequence of SEQ ID NO: 290 and a light chain with an amino acid sequence of SEQ ID NO: 2; or a heavy chain with an amino acid sequence of SEQ ID NO: 3 and optionally an Fc polypeptide with an amino acid sequence of SEQ ID NO: 291 and a light chain with an amino acid sequence of SEQ ID NO: 4; or a heavy chain with an amino acid sequence of SEQ ID NO: 360 and optionally an Fc polypeptide with an amino acid sequence of SEQ ID NO: 392 and a light chain with an amino acid sequence of SEQ ID NO: 361; or a heavy chain with an amino acid sequence of SEQ ID NO: 5 and optionally an Fc polypeptide of an IgG1 isotype (e.g., an amino acid sequence of SEQ ID NO: 283) and a light chain with an amino acid sequence of SEQ ID NO: 6; or a heavy chain with an amino acid sequence of SEQ ID NO: 7 and optionally an Fc polypeptide of an IgG4 isotype (e.g., an amino acid sequence of SEQ ID NO: 285) and a light chain with an amino acid sequence of SEQ ID NO: 8; or a heavy chain with an amino acid sequence of SEQ ID NO: 9 and optionally an Fc polypeptide with an amino acid sequence of SEQ ID NO: 292 and a light chain with an amino acid sequence of SEQ ID NO: 10; or a heavy chain with an amino acid sequence of SEQ ID NO: 11 and optionally an Fc polypeptide of an IgG1 isotype (e.g., an amino acid sequence of SEQ ID NO: 283) and a light chain with an amino acid sequence of SEQ ID NO: 12; or a heavy chain with an amino acid sequence of SEQ ID NO: 13 and optionally an Fc polypeptide of an IgG4 isotype (e.g., an amino acid sequence of SEQ ID NO: 285) and a light chain with an amino acid sequence of SEQ ID NO: 14; or a heavy chain with an amino acid sequence of SEQ ID NO: 15 and optionally an Fc polypeptide with an amino acid sequence of SEQ ID NO: 293 and a light chain with an amino acid sequence of SEQ ID NO: 16; or a heavy chain with an amino acid sequence of SEQ ID NO: 17 and optionally an Fc polypeptide of an IgG1 isotype (e.g., an amino acid sequence of SEQ ID NO: 283) and a light chain with an amino acid sequence of SEQ ID NO: 18; or a heavy chain with an amino acid sequence of SEQ ID NO: 19 and optionally an Fc polypeptide with an amino acid sequence of SEQ ID NO: 294 and a light chain with an amino acid sequence of SEQ ID NO: 20; or a heavy chain with an amino acid sequence of SEQ ID NO: 21 and optionally an Fc polypeptide with an amino acid sequence of SEQ ID NO: 295 and a light chain with an amino acid sequence of SEQ ID NO: 22; or a heavy chain with an amino acid sequence of SEQ ID NO: 23 and optionally an Fc polypeptide of an IgG1 isotype (e.g., an amino acid sequence of SEQ ID NO: 283) and a light chain with an amino acid sequence of SEQ ID NO: 24; or a heavy chain with an amino acid sequence of SEQ ID NO: 25 and optionally an Fc polypeptide with an amino acid sequence of SEQ ID NO: 296 and a light chain with an amino acid sequence of SEQ ID NO: 26; or a heavy chain with an amino acid sequence of SEQ ID NO: 27 and optionally an Fc polypeptide with an amino acid sequence of SEQ ID NO: 297 and a light chain with an amino acid sequence of SEQ ID NO: 28; or a heavy chain with an amino acid sequence of SEQ ID NO: 29 and optionally an Fc polypeptide with an amino acid sequence of SEQ ID NO: 298 and a light chain with an amino acid sequence of SEQ ID NO: 30.

5. The pharmaceutical composition of paragraph 4, wherein the transgene comprises a nucleotide sequence of SEQ ID NO: 71 encoding the heavy chain and a nucleotide sequence of SEQ ID NO: 72 encoding the light chain; or a nucleotide sequence of SEQ ID NO: 73 encoding the heavy chain and a nucleotide sequence of SEQ ID NO: 74 encoding the light chain; or a nucleotide sequence of SEQ ID NO: 376 encoding the heavy chain and a nucleotide sequence of SEQ ID NO: 377 encoding the light chain; or a nucleotide sequence of SEQ ID NO: 75 encoding the heavy chain and a nucleotide sequence of SEQ ID NO: 76 encoding the light chain; or a heavy chain with an nucleotide sequence of SEQ ID NO: 77 and a light chain with an nucleotide sequence of SEQ ID NO: 78; a heavy chain with an nucleotide sequence of SEQ ID NO: 79 and a light chain with an nucleotide sequence of SEQ ID NO: 80; or a heavy chain with an nucleotide sequence of SEQ ID NO: 81 and a light chain with an nucleotide sequence of SEQ ID NO: 82; or a heavy chain with an nucleotide sequence of SEQ ID NO: 83 and a light chain with an nucleotide sequence of SEQ ID NO: 84; or a heavy chain with an nucleotide sequence of SEQ ID NO: 85 and a light chain with an nucleotide sequence of SEQ ID NO: 86; or a heavy chain with an nucleotide sequence of SEQ ID NO: 87 and a light chain with an nucleotide sequence of SEQ ID NO: 88; or a heavy chain with an nucleotide sequence of SEQ ID NO: 89 and a light chain with an nucleotide sequence of SEQ ID NO: 90; or a heavy chain with an nucleotide sequence of SEQ ID NO: 91 and a light chain with an nucleotide sequence of SEQ ID NO: 92; or a heavy chain with an nucleotide sequence of SEQ ID NO: 93 and a light chain with an nucleotide sequence of SEQ ID NO: 94; or a heavy chain with an nucleotide sequence of SEQ ID NO: 95 and a light chain with an nucleotide sequence of SEQ ID NO: 96; or a heavy chain with an nucleotide sequence of SEQ ID NO: 97 and a light chain with an nucleotide sequence of SEQ ID NO: 98; or a heavy chain with an nucleotide sequence of SEQ ID NO: 99 and a light chain with an nucleotide sequence of SEQ ID NO: 100.

6. The pharmaceutical composition of any of paragraphs 1 to 4, wherein the mAb or antigen-binding fragment thereof is a hyperglycosylated mutant or wherein the Fc polypeptide of the mAb is glycosylated or aglycosylated.

7. The pharmaceutical composition of any of paragraphs 1 to 6, wherein the transgene encodes a signal sequence at the N-terminus of the heavy chain and the light chain of said antigen-binding fragment that directs secretion and post translational modification in said human CNS, muscle, or liver cells.

8. The pharmaceutical composition of paragraph 7, wherein said signal sequence is MYRMQLLLLIALSLALVTNS (SEQ ID NO: 146) or a signal sequence from Table 2.

9. The pharmaceutical composition of any of paragraphs 1 to 8, wherein the AAV capsid is AAV8 or AAV9.

10. A pharmaceutical composition for treating retinal disorders including diabetic retinopathy, myopic choroidal neovascularization (mCNV), macular degeneration (e.g., neovascular (wet) or dry age-related macular degeneration (nAMD)), macular edema (e.g., macular edema following a retinal vein occlusion (RVO) or diabetic macular edema (DME)), retinal vein occlusion, diabetic retinopathy (DR), non-infectious uveitis, or glaucoma, or abnormal vascularization of the retina in a human subject in need thereof, comprising an AAV vector comprising:
(a) a viral capsid that is at least 95% identical to the amino acid sequence of an AAV2.7m8 capsid (SEQ ID NO: 142), an AAV8 capsid (SEQ ID NO: 143), an AAV9 capsid (SEQ ID NO: 144); or an AAVrh10 capsid (SEQ ID NO: 145); and
(b) an artificial genome comprising an expression cassette flanked by AAV ITRs wherein the expression cassette comprises a transgene encoding a substantially full-length or full-length mAB of an anti-vascular endothelial growth factor (anti-VEGF), anti-erythropoietin receptor (anti-EPOR), anti-Aβ, anti-activin receptor like kinase 1 (anti-ALK1), anti-complement component 5 (anti-C5), anti-endoglin (anti-ENG), anti-complement component 1Q (anti-CC1Q), or anti-pKal mAb, or an antigen-binding fragment thereof, operably linked to one or more regulatory sequences that control expression of the transgene in human retina cells;
wherein said AAV vector is formulated for subretinal, intravitreal, intranasal, or suprachoroidal administration to the subject.

11. The pharmaceutical composition of paragraph 10, wherein the anti-VEGF mAb is sevacizumab; anti-EPOR mAb is LKA-651 (NSV2) or LKA-651 (NSV3); anti-Aβ mAb is solanezumab, lecanemab, or GSK933776; anti-ALK1 mAb is ascrinvacumab; anti-C5 mAb is tesidolumab or ravulizumab; anti-ENG mAb is carotuximab; the anti-CC1Q mAb is ANX-007; and the anti-pKal mAb is lanadelumab.

12. The pharmaceutical composition of paragraphs 10 or 11, wherein the antigen-binding fragment is a Fab, a F(ab′)₂, or an scFv.

13. The pharmaceutical composition of any of paragraphs 10 to 12, wherein the full-length mAb or the antigen-binding fragment comprises a heavy chain with an amino acid sequence of SEQ ID NO: 1 and optionally an Fc polypeptide with an amino acid sequence of SEQ ID NO: 290 and a light chain with an amino acid sequence of SEQ ID NO: 2; or a heavy chain with an amino acid sequence of SEQ ID NO: 360 and optionally an Fc polypeptide with an amino acid sequence of SEQ ID NO: 392 and a light chain with an amino acid sequence of SEQ ID NO: 361; or a heavy chain with an amino acid sequence of SEQ ID NO: 31 and optionally an Fc polypeptide with an amino acid sequence of SEQ ID NO: 299 and a light chain with an amino acid sequence of SEQ ID NO: 32; or a heavy chain with an amino acid sequence of SEQ ID NO: 33 and optionally an Fc polypeptide of an IgG1 isotype (e.g., an amino acid sequence of SEQ ID NO: 283) and a light chain with an amino acid sequence of SEQ ID NO: 34; or a heavy chain with an amino acid sequence of SEQ ID NO: 35 and optionally an Fc polypeptide of an IgG1 isotype (e.g., an amino acid sequence of SEQ ID NO: 283) and a light chain with an amino acid sequence of SEQ ID NO: 36; or a heavy chain with an amino acid sequence of SEQ ID NO: 3 and optionally an Fc polypeptide with an amino acid sequence of SEQ ID NO: 291 and a light chain with an amino acid sequence of SEQ ID NO: 4; or a heavy chain with an amino acid sequence of SEQ ID NO: 37 and optionally an Fc polypeptide with an amino acid sequence of SEQ ID NO: 300 and a light chain with an amino acid sequence of SEQ ID NO: 38; or a heavy chain with an amino acid sequence of SEQ ID NO: 39 and optionally an Fc polypeptide with an amino acid sequence of SEQ ID NO: 301 and a light chain with an amino acid sequence of SEQ ID NO: 40; or a heavy chain with an amino acid sequence of SEQ ID NO: 362 and optionally an Fc polypeptide with an amino acid sequence of SEQ ID NO: 393 and a light chain with an amino acid sequence of SEQ ID NO: 363; or a heavy chain with an amino acid sequence of SEQ ID NO: 41 and optionally an Fc polypeptide with an amino acid sequence of SEQ ID NO: 302 and a light chain with an amino acid sequence of SEQ ID NO: 42; or a heavy chain with an amino acid sequence of SEQ ID NO: 43 and optionally an Fc polypeptide of an IgG1 isotype (e.g., an amino acid sequence of SEQ ID NO: 283) and a light chain with an amino acid sequence of SEQ ID NO: 44; or a heavy chain with an amino acid sequence of SEQ ID NO: 69 and optionally an Fc polypeptide with an amino acid sequence of SEQ ID NO: 314 and a light chain with an amino acid sequence of SEQ ID NO: 70.

14. The pharmaceutical composition of paragraph 13, wherein the transgene comprises a nucleotide sequence of SEQ ID NO: 71 encoding the heavy chain and a nucleotide sequence of SEQ ID NO: 72 encoding the light chain; or a nucleotide sequence of SEQ ID NO: 376 encoding the heavy chain and a nucleotide sequence of SEQ ID NO: 377 encoding the light chain; or a nucleotide sequence of SEQ ID NO: 101 encoding the heavy chain and a nucleotide sequence of SEQ ID NO: 102 encoding the light chain; or a nucleotide sequence of SEQ ID NO: 103 encoding the heavy chain and a nucleotide sequence of SEQ ID NO: 104 encoding the light chain; or a nucleotide sequence of SEQ ID NO: 105 encoding the heavy chain and a nucleotide sequence of SEQ ID NO: 106 encoding the light chain; or a nucleotide sequence of SEQ ID NO: 73 encoding the heavy chain and a nucleotide sequence of SEQ ID NO: 74 encoding the light chain; or a nucleotide sequence of SEQ ID NO: 107 encoding the heavy chain and a nucleotide sequence of SEQ ID NO: 108 encoding the light chain; or a nucleotide sequence of SEQ ID NO: 109 encoding the heavy chain and a nucleotide sequence of SEQ ID NO: 110 encoding the light chain; or a nucleotide sequence of SEQ ID NO: 378 encoding the heavy chain and a nucleotide sequence of SEQ ID NO: 379 encoding the light chain; or a nucleotide sequence of SEQ ID NO: 111 encoding the heavy chain and a nucleotide sequence of SEQ ID NO: 112 encoding the light chain; or a nucleotide sequence of SEQ ID NO: 113 encoding the heavy chain and a nucleotide sequence of SEQ ID NO: 114 encoding the light chain; or a nucleotide sequence of SEQ ID NO: 139 encoding the heavy chain and a nucleotide sequence of SEQ ID NO: 140 encoding the light chain; or a nucleotide sequence of SEQ ID NO 141, 286, 287, or 435 to 443.

15. The pharmaceutical composition of any of paragraphs 10 to 13, wherein the mAb or antigen-binding fragment thereof is a hyperglycosylated mutant or wherein the Fc polypeptide of the mAb is glycosylated or aglycosylated.

16. The pharmaceutical composition of any of paragraphs 10 to 15, wherein the transgene encodes a signal sequence at the N-terminus of the heavy chain and the light chain of said antigen-binding fragment that directs secretion and post translational modification in said human retina cells.

17. The pharmaceutical composition of paragraph 16, wherein said signal sequence is MYRMQLLLLIALSLALVTNS (SEQ ID NO: 146) or a signal sequence from Table 2, Table 3 or Table 4.

18. The pharmaceutical composition of any of paragraphs 10 to 17, wherein the AAV capsid is AAV8.

19. A pharmaceutical composition for treating non-infectious uveitis in a human subject in need thereof, comprising an AAV vector comprising:
(a) a viral capsid that is at least 95% identical to the amino acid sequence of an AAV2.7m8 (SEQ ID NO: 142), an AAV8 capsid (SEQ ID NO: 143), an AAV9 capsid (SEQ ID NO: 144), or an AAVrh10 capsid (SEQ ID NO: 145); and
(b) an artificial genome comprising an expression cassette flanked by AAV ITRs wherein the expression cassette comprises a transgene encoding a substantially full-length or full-length anti-tumor necrosis factor-alpha (anti-TNFα) mAb or an antigen-binding fragment thereof, a substantially full-length or full-length anti-complement component 5 (C5) mAb or an antigen-binding fragment thereof, a substantially full-length or full-length anti-interleukin-6 (IL-6) mAb or an antigen-binding fragment thereof, or a substantially full-length or full-length anti-interleukin-6 receptor (IL-6R) mAb or an antigen-binding fragment thereof, operably linked to one or more regulatory sequences that control expression of the transgene in human retina cells;
wherein said AAV vector is formulated for subretinal, intravitreal, intranasal, or suprachoroidal administration to the subject.

20. The pharmaceutical composition of paragraph 19 wherein the anti-TNFα mAb is adalimumab, infliximab or golimumab; the anti-C5 mAb is tesidolumab or ravulizumab; the anti-IL-6 mAb is siltuximab, clazakimzumab, sirukumab, olokizumab or gerilimzumab; or the anti-IL-6R mAb is satralizumab, sarilumab or tocilizumab.

21. The pharmaceutical composition of paragraphs 19 or 20, wherein the antigen-binding fragment is a Fab, a F(ab′)₂, or an scFv.

22. The pharmaceutical composition of any of paragraphs 19 to 21, wherein the full-length mAb or the antigen-binding fragment comprises a heavy chain with an amino acid sequence of SEQ ID NO: 45 and optionally an Fc polypeptide with an amino acid sequence of SEQ ID NO: 303 and a light chain with an amino acid sequence of SEQ ID NO: 46, or SEQ ID NO; 451, 452 or 453; or a heavy chain with an amino acid sequence of SEQ ID NO: 47 and optionally an Fc polypeptide with an amino acid sequence of SEQ ID NO: 304 and a light chain with an amino acid sequence of SEQ ID NO: 48; or a heavy chain with an amino acid sequence of SEQ ID NO: 49 and optionally an Fc polypeptide with an amino acid sequence of SEQ ID NO: 305 and a light chain with an amino acid sequence of SEQ ID NO: 50; a heavy chain with an amino acid sequence of SEQ ID NO: 39 and optionally an Fc polypeptide with an amino acid sequence of SEQ ID NO: 301 and a light chain with an amino acid sequence of SEQ ID NO: 40; a heavy chain with an amino acid sequence of SEQ ID NO: 362 and optionally an Fc polypeptide with an amino acid sequence of SEQ ID NO: 393 and a light chain with an amino acid sequence of SEQ ID NO: 363; a heavy chain with an amino acid sequence of SEQ ID NO: 331 and optionally an Fc polypeptide with an amino acid sequence of SEQ ID NO: 355 and a light chain with an amino acid sequence of SEQ ID NO: 332; a heavy chain with an amino acid sequence of SEQ ID NO: 333 and optionally an Fc polypeptide with an amino acid sequence of SEQ ID NO: 356 and a light chain with an amino acid sequence of SEQ ID NO: 334; a heavy chain with an amino acid sequence of SEQ ID NO: 335 and optionally an Fc polypeptide with an amino acid sequence of SEQ ID NO: 357 and a light chain with an amino acid sequence of SEQ ID NO: 336; a heavy chain with an amino acid sequence of SEQ ID NO: 337 and optionally an Fc polypeptide with an amino acid sequence of SEQ ID NO: 358 and a light chain with an amino acid sequence of SEQ ID NO: 338; a heavy chain with an amino acid sequence of SEQ ID NO: 339 and a light chain with an amino acid sequence of SEQ ID NO: 340; a heavy chain with an amino acid sequence of SEQ ID NO: 59 and optionally an Fc polypeptide with an amino acid sequence of SEQ ID NO: 309 and a light chain with an amino acid sequence of SEQ ID NO: 60; a heavy chain with an amino acid sequence of SEQ ID NO: 61 and optionally an Fc polypeptide with an amino acid sequence of SEQ ID NO: 310 and a light chain with an amino acid sequence of SEQ ID NO: 62; and a heavy chain with an amino acid sequence of SEQ ID NO: 341 and optionally an Fc polypeptide with an amino acid sequence of SEQ ID NO: 359 and a light chain with an amino acid sequence of SEQ ID NO: 342.

23. The pharmaceutical composition of paragraph 22, wherein the transgene comprises a nucleotide sequence of SEQ ID NO: 115 encoding the heavy chain and a nucleotide sequence of SEQ ID NO: 116 encoding the light chain; or a nucleotide sequence of SEQ ID NO: 117 encoding the heavy chain and a nucleotide sequence of SEQ ID NO: 118 encoding the light chain; a nucleotide sequence of SEQ ID NO: 119 encoding the heavy chain and a nucleotide sequence of SEQ ID NO: 120 encoding the light chain; nucleotide sequence of SEQ ID NO: 109 encoding the heavy chain and a nucleotide sequence of SEQ ID NO: 110 encoding the light chain; or a nucleotide sequence of SEQ ID NO: 378 encoding the heavy chain and a nucleotide sequence of SEQ ID NO: 379 encoding the light chain; a nucleotide sequence of SEQ ID NO: 343 encoding the heavy chain and a nucleotide sequence of SEQ ID NO: 344 encoding the light chain; nucleotide sequence of SEQ ID NO: 345 encoding the heavy chain and a nucleotide sequence of SEQ ID NO: 346 encoding the light chain; a nucleotide sequence of SEQ ID NO: 347 encoding the heavy chain and a nucleotide sequence of SEQ ID NO: 348 encoding the light chain; a nucleotide sequence of SEQ ID NO: 349 encoding the heavy chain and a nucleotide sequence of SEQ ID NO: 350 encoding the light chain; nucleotide sequence of SEQ ID NO: 351 encoding the heavy chain and a nucleotide sequence of SEQ ID NO: 352 encoding the light chain; a nucleotide sequence of SEQ ID NO: 129 encoding the heavy chain and a nucleotide sequence of SEQ ID NO: 130 encoding the light chain; a nucleotide sequence of SEQ ID NO: 131 encoding the heavy chain and a nucleotide sequence of SEQ ID NO: 132 encoding the light chain; or nucleotide sequence of SEQ ID NO: 341 encoding the heavy chain and a nucleotide sequence of SEQ ID NO: 342 encoding the light chain.

24. The pharmaceutical composition of any of paragraphs 19 to 22, wherein the mAb or antigen-binding fragment thereof is a hyperglycosylated mutant or wherein the Fc polypeptide of the mAb is glycosylated or aglycosylated.

25. The pharmaceutical composition of any of paragraphs 19 to 24, wherein the transgene encodes a signal sequence at the N-terminus of the heavy chain and the light chain of said antigen-binding fragment that directs secretion and post translational modification in said human retina cells.

26. The pharmaceutical composition of paragraph 25, wherein said signal sequence is MYRMQLLLLIALSLALVTNS (SEQ ID NO: 146) or a signal sequence from Table 2, Table 3 or Table 4.

27. The pharmaceutical composition of any of paragraphs 19 to 26, wherein the AAV capsid is AAV8.

28. A pharmaceutical composition for treating multiple sclerosis in a human subject in need thereof, comprising an AAV vector comprising:
(a) a viral capsid that is at least 95% identical to the amino acid sequence of an AAV8 capsid (SEQ ID NO: 143), AAV9 capsid (SEQ ID NO: 144), AAVrh10 capsid (SEQ ID NO: 145), an AAVrh20 capsid, an AAVrh39 capsid or an AAVcy5 capsid; and
(b) an artificial genome comprising an expression cassette flanked by AAV ITRs wherein the expression cassette comprises a transgene encoding a substantially full-length or full-length anti-repulsive guidance molecule-A (anti-RGMa) mAb, or an antigen-binding fragment thereof, operably linked to one or more regulatory sequences that control expression of the transgene in human CNS cells, human liver cells, and/or human muscle cells;
wherein said AAV vector is formulated for administration to the subject, optionally wherein administration is intrathecal, intravenous, subcutaneous, intranasal, or intramuscular.

29. The pharmaceutical composition of paragraph 28 wherein the anti-RGMa mAb is elezanumab.

30. The pharmaceutical composition of paragraphs 28 or 29, wherein the antigen-binding fragment is a Fab, a F(ab′)₂, or an scFv.

31. The pharmaceutical composition of any of paragraphs 28 to 30, wherein the full-length mAb or the antigen-binding fragment comprises a heavy chain with an amino acid sequence of SEQ ID NO: 51 and optionally an Fc polypeptide with an amino acid sequence of SEQ ID NO: 306 and a light chain with an amino acid sequence of SEQ ID NO: 52.

32. The pharmaceutical composition of paragraph 31, wherein the transgene comprises a nucleotide sequence of SEQ ID NO: 121 encoding the heavy chain and a nucleotide sequence of SEQ ID NO: 122 encoding the light chain.

33. The pharmaceutical composition of any of paragraphs 28 to 31, wherein the antibody or antigen-binding fragment thereof is a hyperglycosylated mutant or wherein the Fc polypeptide of the mAb is glycosylated or aglycosylated.

34. The pharmaceutical composition of any of paragraphs 28 to 33, wherein the transgene encodes a signal sequence at the N-terminus of the heavy chain and the light chain of said antigen-binding fragment that directs secretion and post translational modification in said human CNS cells.

35. The pharmaceutical composition of paragraph 34, wherein said signal sequence is MYRMQLLLLIALSLALVTNS (SEQ ID NO: 146) or a signal sequence from Table 2, Table 3 or Table 4.

36. The pharmaceutical composition of any of paragraphs 28 to 35, wherein the AAV capsid is AAV9.

37. A pharmaceutical composition for treating amyloidosis (ATTR), familial amyloid cardiomyopathy (FAC), or familial amyloid polyneuropathy (FAP) in a human subject in need thereof, comprising an AAV vector comprising:
(a) a viral capsid that is at least 95% identical to the amino acid sequence of an AAV8 capsid (SEQ ID NO: 143), an AAV9 capsid (SEQ ID NO: 144), an AAVrh10 capsid (SEQ ID NO: 145); and
(b) an artificial genome comprising an expression cassette flanked by AAV ITRs wherein the expression cassette comprises a transgene encoding a substantially full-length or full-length anti-transthyretin (anti-TTR) mAb, or an antigen-binding fragment thereof, operably linked to one or more regulatory sequences that control expression of the transgene in human liver cells or human muscle cells;
wherein said AAV vector is formulated for subcutaneous, intramuscular or intravenous administration to the subject.

38. The pharmaceutical composition of paragraph 37, wherein the anti-TTR mAb is NI-301 or PRX-004.

39. The pharmaceutical composition of paragraphs 37 or 38, wherein the antigen-binding fragment is a Fab, a F(ab′)₂, or an scFv.

40. The pharmaceutical composition of any of paragraphs 37 to 39, wherein the full-length mAb or the antigen-binding fragment comprises a heavy chain with an amino acid sequence of SEQ ID NO: 53 and optionally an Fc polypeptide of an IgG1 isotype (e.g., an amino acid sequence of SEQ ID NO: 283) and a light chain with an amino acid sequence of SEQ ID NO: 54; or a heavy chain with an amino acid sequence of SEQ ID NO: 55 and optionally an Fc polypeptide with an amino acid sequence of SEQ ID NO: 307 and a light chain with an amino acid sequence of SEQ ID NO: 56.

41. The pharmaceutical composition of paragraph 40, wherein the transgene comprises a nucleotide sequence of SEQ ID NO: 123 encoding the heavy chain and a nucleotide sequence of SEQ ID NO: 124 encoding the light chain; or a nucleotide sequence of SEQ ID NO: 125 encoding the heavy chain and a nucleotide sequence of SEQ ID NO: 126 encoding the light chain.

42. The pharmaceutical composition of any of paragraphs 37 to 41, wherein the mAb or antigen-binding fragment thereof is a hyperglycosylated mutant or wherein the Fc polypeptide of the mAb is glycosylated or aglycosylated.

43. The pharmaceutical composition of any of paragraphs 37 to 42, wherein the transgene encodes a signal sequence at the N-terminus of the heavy chain and the light chain of said antigen-binding fragment that directs secretion and post translational modification in said human liver cells or human muscle cells.

44. The pharmaceutical composition of paragraph 43, wherein said signal sequence is MYRMQLLLLIALSLALVTNS (SEQ ID NO: 146) or a signal sequence from Table 3 or Table 4.

45. The pharmaceutical composition of any of paragraphs 37 to 44, wherein the AAV capsid is AAV8.

46. A pharmaceutical composition for treating fibrotic disorders, pulmonary fibrosis, cystic fibrosis (CF), idiopathic pulmonary fibrosis (IPF), liver cirrhosis, atrial fibrosis, endomyocardial fibrosis, old myocardial infarction, arthrofibrosis, Crohn's disease, ulcerative colitis, mediastinal fibrosis, myelofibrosis (MF), nephrogenic systemic fibrosis (NSF), progressive massive fibrosis (PMF), and retroperitoneal fibrosis (RPF) in a human subject in need thereof, comprising an AAV vector comprising:
(a) a viral capsid that is at least 95% identical to the amino acid sequence of an AAV8 capsid (SEQ ID NO: 143), an AAV9 capsid (SEQ ID NO: 144), or AAVrh10 (SEQ ID NO: 145); and
(b) an artificial genome comprising an expression cassette flanked by AAV ITRs wherein the expression cassette comprises a transgene encoding a substantially full-length or full-length anti-connective tissue growth factor (anti-CTGF) mAb or an antigen-binding fragment thereof, operably linked to one or more regulatory sequences that control expression of the transgene in human liver cells or human muscle cells;
wherein said AAV vector is formulated for subcutaneous, intramuscular or intravenous administration to the subject.

47. The pharmaceutical composition of paragraph 46, wherein the anti-CTGF mAb is pamrevlumab.

48. The pharmaceutical composition of paragraphs 46 or 47, wherein the antigen-binding fragment is a Fab, a F(ab′)₂, or an scFv.

49. The pharmaceutical composition of any of paragraphs 46 to 48, wherein the full-length mAb or the antigen-binding fragment comprises a heavy chain with an amino acid sequence of SEQ ID NO: 57 and optionally an Fc polypeptide with an amino acid sequence of SEQ ID NO: 308 and a light chain with an amino acid sequence of SEQ ID NO: 58.

50. The pharmaceutical composition of paragraph 49, wherein the transgene comprises a nucleotide sequence of SEQ ID NO: 127 encoding the heavy chain and a nucleotide sequence of SEQ ID NO: 128 encoding the light chain.

51. The pharmaceutical composition of any of paragraphs 44 to 50, wherein the mAb or antigen-binding fragment thereof is a hyperglycosylated mutant or wherein the Fc polypeptide of the mAb is glycosylated or aglycosylated.

52. The pharmaceutical composition of any of paragraphs 44 to 51, wherein the transgene encodes a signal sequence at the N-terminus of the heavy chain and the light chain of said antigen-binding fragment that directs secretion and post translational modification in said human liver cells or human muscle cells.

53. The pharmaceutical composition of paragraph 52, wherein said signal sequence is MYRMQLLLLIALSLALVTNS (SEQ ID NO: 146) or a signal sequence from Table 3 or Table 4.

54. The pharmaceutical composition of any of paragraphs 44 to 53, wherein the AAV capsid is AAV8.

55. A pharmaceutical composition for treating non-infectious uveitis, neuromyelitis optica (NMO), diabetic retinopathy (DR), or diabetic macular edema (DME) in a human subject in need thereof, comprising an AAV vector comprising:
(a) a viral capsid that is at least 95% identical to the amino acid sequence of an AAV8 capsid (SEQ ID NO: 143), an AAV2.7m8 capsid (SEQ ID NO: 142), an AAV9 capsid (SEQ ID NO: 144), or an AAVrh10 capsid (SEQ ID NO: 145); and
(b) an artificial genome comprising an expression cassette flanked by AAV ITRs wherein the expression cassette comprises a transgene encoding a substantially full-length or full-length mAb of an anti-interleukin-6 receptor (anti-IL6R), anti-interleukin-6 (IL6), or anti-cluster of differentiation 19 (anti-CD19) mAb, or an antigen-binding fragment thereof, operably linked to one or more regulatory sequences that control expression of the transgene in human retina cells;
wherein said AAV vector is formulated for subretinal, intravitreal, intranasal, or suprachoroidal administration to the subject.

56. The pharmaceutical composition of paragraph 55, wherein the anti-IL6R mAb is satralizumab, sarilumab, or tocilizumab, or the anti-IL6 mAb is siltuximab, clazakizumab, sirukumab, olokizumab, or gerilimzumab, or the anti-CD19 mAb is inebilizumab.

57. The pharmaceutical composition of paragraphs 55 or 56, wherein the antigen-binding fragment is a Fab, a F(ab′)₂, or an scFv.

58. The pharmaceutical composition of any of paragraphs 55 to 57, wherein the full-length mAb or the antigen-binding fragment comprises a heavy chain with an amino acid sequence of SEQ ID NO: 59 and optionally an Fc polypeptide with an amino acid sequence of SEQ ID NO: 309 and a light chain with an amino acid sequence of SEQ ID NO: 60; or a heavy chain with an amino acid sequence of SEQ ID NO: 61 and optionally an Fc polypeptide with an amino acid sequence of SEQ ID NO: 310 and a light chain with an amino acid sequence of SEQ ID NO: 62; or a heavy chain with an amino acid sequence of SEQ ID NO: 331 and optionally an Fc polypeptide with an amino acid sequence of SEQ ID NO: 355 and a light chain with an amino acid sequence of SEQ ID NO: 332; or a heavy chain with an amino acid sequence of SEQ ID NO: 333 and optionally an Fc polypeptide with an amino acid sequence of SEQ ID NO: 356 and a light chain with an amino acid sequence of SEQ ID NO: 334; or a heavy chain with an amino acid sequence of SEQ ID NO: 335 and optionally an Fc polypeptide with an amino acid sequence of SEQ ID NO: 357 and a light chain with an amino acid sequence of SEQ ID NO: 336; or a heavy chain with an amino acid sequence of SEQ ID NO: 337 and optionally an Fc polypeptide with an amino acid sequence of SEQ ID NO: 358 and a light chain with an amino acid sequence of SEQ ID NO: 338; or a heavy chain with an amino acid sequence of SEQ ID NO: 339 and optionally an Fc polypeptide with an amino acid sequence of SEQ ID NO: 283 and a light chain with an amino acid sequence of SEQ ID NO: 340; or a heavy chain with an amino acid sequence of SEQ ID NO: 341 and optionally an Fc polypeptide with an amino acid sequence of SEQ ID NO: 359 and a light chain with an amino acid sequence of SEQ ID NO: 342; a heavy chain with an amino acid sequence of SEQ ID NO: 63 and optionally an Fc polypeptide with an amino acid sequence of SEQ ID NO: 311 and a light chain with an amino acid sequence of SEQ ID NO: 64.

59. The pharmaceutical composition of paragraph 58, wherein the transgene comprises a nucleotide sequence of SEQ ID NO: 129 encoding the heavy chain and a nucleotide sequence of SEQ ID NO: 130 encoding the light chain; or a nucleotide sequence of SEQ ID NO: 131 encoding the heavy chain and a nucleotide sequence of SEQ ID NO: 132 encoding the light chain; or a nucleotide sequence of SEQ ID NO: 343 encoding the heavy chain and a nucleotide sequence of SEQ ID NO: 344 encoding the light chain; or a nucleotide sequence of SEQ ID NO: 345 encoding the heavy chain and a nucleotide sequence of SEQ ID NO: 346 encoding the light chain; or a nucleotide sequence of SEQ ID NO: 347 encoding the heavy chain and a nucleotide sequence of SEQ ID NO: 348 encoding the light chain; or a nucleotide sequence of SEQ ID NO: 349 encoding the heavy chain and a nucleotide sequence of SEQ ID NO: 350 encoding the light chain; or a nucleotide sequence of SEQ ID NO: 351 encoding the heavy chain and a nucleotide sequence of SEQ ID NO: 352 encoding the light chain; or a nucleotide sequence of SEQ ID NO: 353 encoding the heavy chain and a nucleotide sequence of SEQ ID NO: 354 encoding the light chain; or a nucleotide sequence of SEQ ID NO: 133 encoding the heavy chain and a nucleotide sequence of SEQ ID NO: 134 encoding the light chain.

60. The pharmaceutical composition of any of paragraphs 55 to 59, wherein the mAb or antigen-binding fragment thereof is a hyperglycosylated mutant or wherein the Fc polypeptide of the mAb is glycosylated or aglycosylated.

61. The pharmaceutical composition of any of paragraphs 55 to 60, wherein the transgene encodes a signal sequence at the N-terminus of the heavy chain and the light chain of said antigen-binding fragment that directs secretion and post translational modification in said human retinal cells.

62. The pharmaceutical composition of paragraph 61, wherein said signal sequence is MYRMQLLLLIALSLALVTNS (SEQ ID NO: 146) or a signal sequence from Table 2, Table 3 or Table 4.

63. The pharmaceutical composition of any of paragraphs 55 to 62, wherein the AAV capsid is AAV8.

64. A pharmaceutical composition for treating inflammatory bowel disease (IBD) including UC and CD in a human subject in need thereof, comprising an AAV vector comprising:
(a) a viral capsid that is at least 95% identical to the amino acid sequence of an AAV8 capsid (SEQ ID NO: 143); AAV9 capsid (SEQ ID NO: 144); or AAVrh10 capsid (SEQ ID NO: 145); and
(b) an artificial genome comprising an expression cassette flanked by AAV ITRs wherein the expression cassette comprises a substantially full-length or full-length anti-integrin β7 subunit (anti-ITGB7) mAb or an antigen-binding fragment thereof, operably linked to one or more regulatory sequences that control expression of the transgene in human liver cells or human muscle cells;
wherein said AAV vector is formulated for subcutaneous, intramuscular, or intravenous administration to the subject.

65. The pharmaceutical composition of paragraph 64, wherein the anti-ITGB7 mAb is etrolizumab.

66. The pharmaceutical composition of paragraphs 64 or 65, wherein the antigen-binding fragment is a Fab, a F(ab′)₂, or an scFv.

67. The pharmaceutical composition of any of paragraphs 64 to 66, wherein the full-length mAb or the antigen-binding fragment comprises a heavy chain with an amino acid sequence of SEQ ID NO: 65 and optionally an Fc polypeptide with an amino acid sequence of SEQ ID NO: 312 and a light chain with an amino acid sequence of SEQ ID NO: 66.

68. The pharmaceutical composition of paragraph 67, wherein the transgene comprises a nucleotide sequence of SEQ ID NO: 135 encoding the heavy chain and a nucleotide sequence of SEQ ID NO: 136 encoding the light chain.

69. The pharmaceutical composition of any of paragraphs 64 to 68, wherein the mAb or antigen-binding fragment thereof is a hyperglycosylated mutant or wherein the Fc polypeptide of the mAb is glycosylated or aglycosylated.

70. The pharmaceutical composition of any of paragraphs 64 to 69, wherein the transgene encodes a signal sequence at the N-terminus of the heavy chain and the light chain of said antigen-binding fragment that directs secretion and post translational modification in said human liver cells or human muscle cells.

71. The pharmaceutical composition of paragraph 70, wherein said signal sequence is MYRMQLLLLIALSLALVTNS (SEQ ID NO: 146) or a signal sequence from Table 3 or Table 4.

72. The pharmaceutical composition of any of paragraphs 64 to 71, wherein the AAV capsid is AAV8.

73. A pharmaceutical composition for treating osteoporosis or abnormal bone loss or weakness (e.g., treating giant cell tumor of bone, treating treatment-induced bone loss, slowing the loss of (or increasing) bone mass in breast and prostate cancer patients, preventing skeletal-related events due to bone metastasis, or for decreasing bone resorption and turnover in a human subject in need thereof, comprising an AAV vector comprising:
(a) a viral capsid that is at least 95% identical to the amino acid sequence of an AAV8 capsid (SEQ ID NO: 143); AAVrh10 capsid (SEQ ID NO: 145); or an AAV9 capsid (SEQ ID NO: 144); and
(b) an artificial genome comprising an expression cassette flanked by AAV ITRs wherein the expression cassette comprises a transgene encoding a substantially full-length or full-length anti-sclerostin (anti-SOST) mAb or an antigen-binding fragment thereof, operably linked to one or more regulatory sequences that control expression of the transgene in human liver cells or human muscle cells;
wherein said AAV vector is formulated for intravenous, intramuscular, or subcutaneous administration to the subject.

74. The pharmaceutical composition of paragraph 73, wherein the anti-SOST mAb is romosozumab.

75. The pharmaceutical composition of paragraphs 73 or 74, wherein the antigen-binding fragment is a Fab, a F(ab′)₂, or an scFv.

76. The pharmaceutical composition of any of paragraphs 73 to 75, wherein the full-length mAb or the antigen-binding fragment comprises a heavy chain with an amino acid sequence of SEQ ID NO: 67 and optionally an Fc polypeptide with an amino acid sequence of SEQ ID NO: 313 and a light chain with an amino acid sequence of SEQ ID NO: 68.

77. The pharmaceutical composition of paragraph 76, wherein the transgene comprises a nucleotide sequence of SEQ ID NO: 137 encoding the heavy chain and a nucleotide sequence of SEQ ID NO: 138 encoding the light chain.

78. The pharmaceutical composition of any of paragraphs 73 to 77, wherein the mAb or antigen-binding fragment thereof is a hyperglycosylated mutant or wherein the Fc polypeptide of the mAb is glycosylated or aglycosylated.

79. The pharmaceutical composition of any of paragraphs 73 to 78, wherein the transgene encodes a signal sequence at the N-terminus of the heavy chain and the light chain of said antigen-binding fragment that directs secretion and post translational modification in said human liver cells or human muscle cells.

80. The pharmaceutical composition of paragraph 79, wherein said signal sequence is MYRMQLLLLIALSLALVTNS (SEQ ID NO: 146) or a signal sequence from Table 3 or Table 4.

81. The pharmaceutical composition of any of paragraphs 73 to 80, wherein the AAV capsid is AAV8.

82. A pharmaceutical composition for treating angioedema including hereditary angioedema in a human subject in need thereof, comprising an AAV vector comprising:
(a) a viral capsid that is at least 95% identical to the amino acid sequence of an AAV8 capsid (SEQ ID NO: 143); AAVrh10 capsid (SEQ ID NO: 145); or AAV9 capsid (SEQ ID NO: 144); and
(b) an artificial genome comprising an expression cassette flanked by AAV ITRs wherein the expression cassette comprises a transgene encoding a substantially full-length or full-length anti-kallikrein (anti-pKal) mAb or an antigen-binding fragment thereof, operably linked to one or more regulatory sequences that control expression of the transgene in human liver cells or human muscle cells;
wherein said AAV vector is formulated for intravenous, intramuscular, or subcutaneous administration to the subject.

83. The pharmaceutical composition of paragraph 82, wherein the anti-pKal mAb is lanadelumab.

84. The pharmaceutical composition of paragraphs 82 or 83, wherein the antigen-binding fragment is a Fab, a F(ab′)₂, or an scFv.

85. The pharmaceutical composition of any of paragraphs 82 to 84, wherein the full-length mAb or the antigen-binding fragment comprises a heavy chain with an amino acid sequence of SEQ ID NO: 69 and optionally an Fc polypeptide with an amino acid sequence of SEQ ID NO: 314 and a light chain with an amino acid sequence of SEQ ID NO: 70.

86. The pharmaceutical composition of paragraph 85, wherein the transgene comprises a nucleotide sequence of SEQ ID NO: 139 encoding the heavy chain and a nucleotide sequence of SEQ ID NO: 140 encoding the light chain; or a nucleotide sequence of SEQ ID NO 141, 286, 287, or 435 to 443.

87. The pharmaceutical composition of any of paragraphs 82 to 85, wherein the mAb or antigen-binding fragment thereof is a hyperglycosylated mutant or wherein the Fc polypeptide of the mAb is glycosylated or aglycosylated.

88. The pharmaceutical composition of any of paragraphs 82 to 87, wherein the transgene encodes a signal sequence at the N-terminus of the heavy chain and the light chain of said antigen-binding fragment that directs secretion and post translational modification in said human retinal cells.

89. The pharmaceutical composition of paragraph 88, wherein said signal sequence is MYRMQLLLLIALSLALVTNS (SEQ ID NO: 146) or a signal sequence from Table 3 or Table 4.

90. The pharmaceutical composition of any of paragraphs 82 to 89, wherein the AAV capsid is AAV8.

### Method of Treatment

91. A method of treating Alzheimer's disease (AD), frontotemporal dementia (FD), tauopathies, progressive supranuclear palsy, chronic traumatic encephalopathy, Pick's Complex, and primary age-related tauopathy, Huntington's disease, juvenile Huntington's disease, Parkinson's disease, synucleinopathies, ALS, migraines or cluster headaches in a human subject in need thereof, comprising delivering to the cerebrospinal fluid (CSF) of said human subject, a therapeutically effective amount of a substantially full-length or full-length mAb of an anti-amyloid beta (anti-AP), anti-sortilin, anti-Tau protein (anti-Tau), anti-semaphorin 4D (anti-SEMA4D), anti-alpha synuclein (anti-SNCA), anti-superoxide dismutase-1 (anti-SOD1) or anti-calcitonin gene-related peptide receptor (anti-CGRPR) mAb, or antigen-binding fragment thereof, expressed from a transgene and produced by human CNS cells.

92. A method of treating Alzheimer's disease, frontotemporal dementia (FD), tauopathies, progressive supranuclear palsy, chronic traumatic encephalopathy, Pick's Complex, and primary age-related tauopathy, Huntington's disease, juvenile Huntington's disease, Parkinson's disease, synucleinopathies, ALS, migraines or cluster headaches in a human subject in need thereof, comprising:
administering to said subject a therapeutically effective amount of a recombinant nucleotide expression vector comprising a transgene encoding a substantially full-length or full-length mAb of an anti-amyloid beta (anti-Aβ), anti-sortilin, anti-Tau protein (anti-Tau), anti-semaphorin 4D (anti-SEMA4D), anti-alpha synuclein (anti-SNCA), anti-superoxide dismutase-1 (anti-SOD1) or anti-calcitonin gene-related peptide receptor (anti-CGRPR) mAb, or an antigen-binding fragment thereof, operably linked to one or more regulatory sequences that control expression of the transgene in human CNS cells, so that a depot is formed that releases a human post-translationally modified (HuPTM) form of said mAb or antigen-binding fragment thereof.

93. The method of paragraphs 91 or 92 wherein the anti-Aβ mAb is solanezumab, lecanemab, or GSK933776; the anti-sortilin mAb is AL-001; the anti-Tau mAb is ABBV-8E12, UCB-0107, or NI-105 (BIIB076); the anti-SEMA4D mAb is VX15/2503; the anti-SNCA mAb is prasinezumab, NI-202 (BIIB054), or MED-1341; the anti-SOD1 mAb is NI-2041.10D12 or NI-204.12G7; and the anti-CGRPR mAb is eptinezumab, fremanezumab, or galcanezumab.

94. The method of any of paragraphs 91 to 93 wherein the antigen-binding fragment is a Fab, a F(ab′)₂, or an scFv.

95. The method of any of paragraphs 91 to 94, wherein the full-length mAb or the antigen-binding fragment comprises a heavy chain with an amino acid sequence of SEQ ID NO: 1 and optionally an Fc polypeptide with an amino acid sequence of SEQ ID NO: 290 and a light chain with an amino acid sequence of SEQ ID NO: 2; or a heavy chain with an amino acid sequence of SEQ ID NO: 3 and optionally an Fc polypeptide with an amino acid sequence of SEQ ID NO: 292 and a light chain with an amino acid sequence of SEQ ID NO: 4; or a heavy chain with an amino acid sequence of SEQ ID NO: 360 and optionally an Fc polypeptide with an amino acid sequence of SEQ ID NO: 392 and a light chain with an amino acid sequence of SEQ ID NO: 361; or a heavy chain with an amino acid sequence of SEQ ID NO: 5 and optionally an Fc polypeptide of an IgG1 isotype (e.g., an amino acid sequence of SEQ ID NO: 283) and a light chain with an amino acid sequence of SEQ ID NO: 6; or a heavy chain with an amino acid sequence of SEQ ID NO: 7 and optionally an Fc polypeptide of an IgG4 isotype (e.g., an amino acid sequence of SEQ ID NO: 285) and a light chain with an amino acid sequence of SEQ ID NO: 8; or a heavy chain with an amino acid sequence of SEQ ID NO: 9 and optionally an Fc polypeptide with an amino acid sequence of SEQ ID NO: 292 and a light chain with an amino acid sequence of SEQ ID NO: 10; or a heavy chain with an amino acid sequence of SEQ ID NO: 11 and optionally an Fc polypeptide of an IgG1 isotype (e.g., an amino acid sequence of SEQ ID NO: 283) and a light chain with an amino acid sequence of SEQ ID NO: 12; or a heavy chain with an amino acid sequence of SEQ ID NO: 13 and optionally an Fc polypeptide of an IgG4 isotype (e.g., an amino acid sequence of SEQ ID NO: 285) and a light chain with an amino acid sequence of SEQ ID NO: 14; or a heavy chain with an amino acid sequence of SEQ ID NO: 15 and optionally an Fc polypeptide with an amino acid sequence of SEQ ID NO: 293 and a light chain with an amino acid sequence of SEQ ID NO: 16; or a heavy chain with an amino acid sequence of SEQ ID NO: 17 and optionally an Fc polypeptide of an IgG1 isotype (e.g., an amino acid sequence of SEQ ID NO: 283) and a light chain with an amino acid sequence of SEQ ID NO: 18; or a heavy chain with an amino acid sequence of SEQ ID NO: 19 and optionally an Fc polypeptide with an amino acid sequence of SEQ ID NO: 294 and a light chain with an amino acid sequence of SEQ ID NO: 20; or a heavy chain with an amino acid sequence of SEQ ID NO: 21 and optionally an Fc polypeptide with an amino acid sequence of SEQ ID NO: 295 and a light chain with an amino acid sequence of SEQ ID NO: 22; or a heavy chain with an amino acid sequence of SEQ ID NO: 23 and optionally an Fc polypeptide of an IgG1 isotype (e.g., an amino acid sequence of SEQ ID NO: 283) and a light chain with an amino acid sequence of SEQ ID NO: 24; or a heavy chain with an amino acid sequence of SEQ ID NO: 25 and optionally an Fc polypeptide with an amino acid sequence of SEQ ID NO: 296 and a light chain with an amino acid sequence of SEQ ID NO: 26; or a heavy chain with an amino acid sequence of SEQ ID NO: 27 and optionally an Fc polypeptide with an amino acid sequence of SEQ ID NO: 297 and a light chain with an amino acid sequence of SEQ ID NO: 28; or a heavy chain with an amino acid sequence of SEQ ID NO: 29 and optionally an Fc polypeptide with an amino acid sequence of SEQ ID NO: 298 and a light chain with an amino acid sequence of SEQ ID NO: 30.

96. The method of paragraph 95, wherein the transgene comprises a nucleotide sequence of SEQ ID NO: 71 encoding the heavy chain and a nucleotide sequence of SEQ ID NO: 72 encoding the light chain; or a nucleotide sequence of SEQ ID NO: 73 encoding the heavy chain and a nucleotide sequence of SEQ ID NO: 74 encoding the light chain; or a nucleotide sequence of SEQ ID NO: 376 encoding the heavy chain and a nucleotide sequence of SEQ ID NO: 377 encoding the light chain; or a nucleotide sequence of SEQ ID NO: 75 encoding the heavy chain and a nucleotide sequence of SEQ ID NO: 76 encoding the light chain; or a heavy chain with an nucleotide sequence of SEQ ID NO: 77 and a light chain with an nucleotide sequence of SEQ ID NO: 78; a heavy chain with an nucleotide sequence of SEQ ID NO: 79 and a light chain with an nucleotide sequence of SEQ ID NO: 80; or a heavy chain with an nucleotide sequence of SEQ ID NO: 81 and a light chain with an nucleotide sequence of SEQ ID NO: 82; or a heavy chain with an nucleotide sequence of SEQ ID NO: 83 and a light chain with an nucleotide sequence of SEQ ID NO: 84; or a heavy chain with an nucleotide sequence of SEQ ID NO: 85 and a light chain with an nucleotide sequence of SEQ ID NO: 86; or a heavy chain with an nucleotide sequence of SEQ ID NO: 87 and a light chain with an nucleotide sequence of SEQ ID NO: 88; or a heavy chain with an nucleotide sequence of SEQ ID NO: 89 and a light chain with an nucleotide sequence of SEQ ID NO: 90; or a heavy chain with an nucleotide sequence of SEQ ID NO: 91 and a light chain with an nucleotide sequence of SEQ ID NO: 92; or a heavy chain with an nucleotide sequence of SEQ ID NO: 93 and a light chain with an nucleotide sequence of SEQ ID NO: 94; or a heavy chain with an nucleotide sequence of SEQ ID NO: 95 and a light chain with an nucleotide sequence of SEQ ID NO: 96; or a heavy chain with an nucleotide sequence of SEQ ID NO: 97 and a light chain with an nucleotide sequence of SEQ ID NO: 98; or a heavy chain with an nucleotide sequence of SEQ ID NO: 99 and a light chain with an nucleotide sequence of SEQ ID NO: 100.

97. The method of any of paragraphs 91 to 95, wherein the mAb or antigen-binding fragment thereof is a hyperglycosylated mutant or wherein the Fc polypeptide of the mAb is glycosylated or aglycosylated.

98. The method of any of paragraphs 91 to 97 wherein the mAb or antigen-binding fragment thereof contains an alpha 2,6-sialylated glycan.

99. The method of any of paragraphs 91 to 98 wherein the mAb or antigen-binding fragment thereof is glycosylated but does not contain detectable NeuGc and/or α-Gal.

100. The method of any of paragraphs 91 to 99 wherein the mAb or antigen-binding fragment thereof contains a tyrosine sulfation.

101. The method of any of paragraphs 92 to 100 wherein the recombinant expression vector is AAV9.

102. The method of any of paragraphs 92 to 101 in which production of said HuPTM form of said mAb or antigen-binding fragment thereof is confirmed by transducing human CNS cells in culture with said recombinant nucleotide expression vector and expressing said mAb or antigen-binding fragment thereof.

103. A method of treating diabetic retinopathy, myopic choroidal neovascularization (mCNV), macular degeneration (e.g., neovascular (wet) or dry age-related macular degeneration (nAMD)), macular edema (e.g., macular edema following a retinal vein occlusion (RVO) or diabetic macular edema (DME)), RVO, diabetic retinopathy (DR), non-infectious uveitis, glaucoma, or abnormal vascularization of the retina in a human subject in need thereof, comprising delivering to the retina of said human subject, a therapeutically effective amount of a substantially full-length or full-length mAb of an anti-vascular endothelial growth factor (anti-VEGF), anti-erythropoietin receptor (anti-EPOR), anti-Aβ, anti-activin receptor like kinase 1 (anti-ALK1), anti-complement component 5 (anti-C5), anti-endoglin (anti-ENG), anti-complement component 1Q (anti-CC1Q)), or anti-pKal mAb, or antigen-binding fragment thereof, expressed from a transgene and produced by human retina cells.

104. A method of treating diabetic retinopathy, myopic choroidal neovascularization (mCNV), macular degeneration (e.g., neovascular (wet) or dry age-related macular degeneration (nAMD)), macular edema (e.g., macular edema following a retinal vein occlusion (RVO) or diabetic macular edema (DME)), RVO, diabetic retinopathy (DR), non-infectious uveitis, glaucoma or abnormal vascularization of the retina in a human subject in need thereof, comprising:
administering to the retina of said subject a therapeutically effective amount of a recombinant nucleotide expression vector comprising a transgene encoding a substantially full-length or full-length mAb of an anti-vascular endothelial growth factor (anti-VEGF), anti-erythropoietin receptor (anti-EPOR), anti-Aβ, anti-activin receptor like kinase 1 (anti-ALK1), anti-complement component 5 (anti-C5), anti-endoglin (anti-ENG), anti-complement component 1Q (anti-CC1Q), or anti-pKal mAb, or an antigen-binding fragment thereof, operably linked to one or more regulatory sequences that control expression of the transgene in human retina cells, so that a depot is formed that releases a HuPTM form of mAb or antigen-binding fragment thereof.

105. The method of paragraph 103 or 104 wherein the anti-VEGF mAb is sevacizumab; anti-EPOR mAb is LKA-651 (NSV2) or LKA-651 (NSV3); anti-Aβ mAb is solanezumab, lecanemab, or GSK933776; anti-ALK1 mAb is ascrinvacumab; anti-C5 mAb is tesidolumab or ravulizumab; anti-ENG mAb is carotuximab; the anti-CC1Q mAb is ANX-007; and the anti-pKal mAb is lanadelumab.

106. The method of any of paragraphs 103 to 105 wherein the antigen-binding fragment is a Fab, a F(ab′)₂, or an scFv.

107. The method of any of paragraphs 103 to 106, wherein the full-length mAb or the antigen-binding fragment comprises a heavy chain with an amino acid sequence of SEQ ID NO: 1 and optionally an Fc polypeptide with an amino acid sequence of SEQ ID NO: 290 and a light chain with an amino acid sequence of SEQ ID NO: 2; or a heavy chain with an amino acid sequence of SEQ ID NO: 360 and optionally an Fc polypeptide with an amino acid sequence of SEQ ID NO: 392 and a light chain with an amino acid sequence of SEQ ID NO: 361; or a heavy chain with an amino acid sequence of SEQ ID NO: 31 and optionally an Fc polypeptide with an amino acid sequence of SEQ ID NO: 299 and a light chain with an amino acid sequence of SEQ ID NO: 32; or a heavy chain with an amino acid sequence of SEQ ID NO: 33 and optionally an Fc polypeptide of an IgG1 isotype (e.g., an amino acid sequence of SEQ ID NO: 283) and a light chain with an amino acid sequence of SEQ ID NO: 34; or a heavy chain with an amino acid sequence of SEQ ID NO: 35 and optionally an Fc polypeptide of an IgG1 isotype (e.g., an amino acid sequence of SEQ ID NO: 283) and a light chain with an amino acid sequence of SEQ ID NO: 36; or a heavy chain with an amino acid sequence of SEQ ID NO: 3 and optionally an Fc polypeptide with an amino acid sequence of SEQ ID NO: 291 and a light chain with an amino acid sequence of SEQ ID NO: 4; or a heavy chain with an amino acid sequence of SEQ ID NO: 37 and optionally an Fc polypeptide with an amino acid sequence of SEQ ID NO: 300 and a light chain with an amino acid sequence of SEQ ID NO: 38; or a heavy chain with an amino acid sequence of SEQ ID NO: 39 and optionally an Fc polypeptide with an amino acid sequence of SEQ ID NO: 301 and a light chain with an amino acid sequence of SEQ ID NO: 40; or a heavy chain with an amino acid sequence of SEQ ID NO: 362 and optionally an Fc polypeptide with an amino acid sequence of SEQ ID NO: 393 and a light chain with an amino acid sequence of SEQ ID NO: 363; or a heavy chain with an amino acid sequence of SEQ ID NO: 41 and optionally an Fc polypeptide with an amino acid sequence of SEQ ID NO: 302 and a light chain with an amino acid sequence of SEQ ID NO: 42; or a heavy chain with an amino acid sequence of SEQ ID NO: 43 and optionally an Fc polypeptide of an IgG1 isotype (e.g., an amino acid sequence of SEQ ID NO: 283) and a light chain with an amino acid sequence of SEQ ID NO: 44; or a heavy chain with an amino acid sequence of SEQ ID NO: 69 and optionally an Fc polypeptide with an amino acid sequence of SEQ ID NO: 314 and a light chain with an amino acid sequence of SEQ ID NO: 70.

108. The method of paragraph 107, wherein the transgene comprises a nucleotide sequence of SEQ ID NO: 71 encoding the heavy chain and a nucleotide sequence of SEQ ID NO: 72 encoding the light chain; or a nucleotide sequence of SEQ ID NO: 376 encoding the heavy chain and a nucleotide sequence of SEQ ID NO: 377 encoding the light chain; or a nucleotide sequence of SEQ ID NO: 101 encoding the heavy chain and a nucleotide sequence of SEQ ID NO: 102 encoding the light chain; or a nucleotide sequence of SEQ ID NO: 103 encoding the heavy chain and a nucleotide sequence of SEQ ID NO: 104 encoding the light chain; or a nucleotide sequence of SEQ ID NO: 105 encoding the heavy chain and a nucleotide sequence of SEQ ID NO: 106 encoding the light chain; or a nucleotide sequence of SEQ ID NO: 73 encoding the heavy chain and a nucleotide sequence of SEQ ID NO: 74 encoding the light chain; or a nucleotide sequence of SEQ ID NO: 107 encoding the heavy chain and a nucleotide sequence of SEQ ID NO: 108 encoding the light chain; or a nucleotide sequence of SEQ ID NO: 109 encoding the heavy chain and a nucleotide sequence of SEQ ID NO: 110 encoding the light chain; or a nucleotide sequence of SEQ ID NO: 378 encoding the heavy chain and a nucleotide sequence of SEQ ID NO: 379 encoding the light chain; or a nucleotide sequence of SEQ ID NO: 111 encoding the heavy chain and a nucleotide sequence of SEQ ID NO: 112 encoding the light chain; or a nucleotide sequence of SEQ ID NO: 113 encoding the heavy chain and a nucleotide sequence of SEQ ID NO: 114 encoding the light chain, or a nucleotide sequence of SEQ ID NO 141, 286, 287, or 435 to 443.

109. The method of any of paragraphs 103 to 105, wherein the mAb or antigen-binding fragment thereof is a hyperglycosylated mutant or wherein the Fc polypeptide of the mAb is glycosylated or aglycosylated.

110. The method of any of paragraphs 103 to 109 wherein the mAb or antigen-binding fragment thereof contains an alpha 2,6-sialylated glycan.

111. The method of any of paragraphs 103 to 110 wherein the mAb or antigen-binding fragment thereof is glycosylated but does not contain detectable NeuGc or α-Gal.

112. The method of any of paragraphs 103 to 111 wherein the mAb or antigen-binding fragment thereof contains a tyrosine sulfation.

113. The method of any of paragraphs 104 to 112 wherein the recombinant expression vector is AAV2.7m8, AAV8, or AAV9.

114. The method of any of paragraphs 104 to 113 in which production of said HuPTM form of the mAb or antigen-binding fragment thereof is confirmed by transducing human retinal cells in culture with said recombinant nucleotide expression vector and expressing said mAb or antigen-binding fragment thereof.

115. A method of treating non-infectious uveitis in a human subject in need thereof, comprising delivering to the retina of said human subject, a therapeutically effective amount of a substantially full-length or full-length anti-tumor necrosis factor-alpha (anti-TNFα) mAb or antigen-binding fragment thereof, expressed from a transgene and produced by human retina cells.

116. A method of treating non-infectious uveitis in a human subject in need thereof, comprising:
administering to the retina of said human subject a therapeutically effective amount of a recombinant nucleotide expression vector comprising a transgene encoding a substantially full-length or full-length anti-tumor necrosis factor-alpha (anti-TNFα) mAb, or an antigen-binding fragment thereof, a substantially full-length or full-length anti-complement component 5 (C5) mAb or an antigen-binding fragment thereof, a substantially full-length or full-length anti-interleukin-6 (IL-6) mAb or an antigen-binding fragment thereof, a substantially full-length or full-length anti-interleukin-6 receptor (IL-6R) mAb or an antigen-binding fragment thereof, operably linked to one or more regulatory sequences that control expression of the transgene in human retina cells, so that a depot is formed that releases a HuPTM form of said mAb or antigen-binding fragment.

117. The method of paragraphs 115 or 116 wherein the anti-TNFα mAb is adalimumab, infliximab or golimumab the anti-C5 mAb is tesidolumab or ravulizumab; the anti-IL-6 mAb is siltuximab, clazakimzumab, sirukumab, olokizumab or gerilimzumab; or the anti-IL-6R mAb is satralizumab, sarilumab or tocilizumab.

118. The method of any of paragraphs 115 to 117 wherein the antigen-binding fragment is a Fab, a F(ab′)₂, or an scFv.

119. The method of any of paragraphs 115 to 118, wherein the full-length mAb or the antigen-binding fragment comprises a heavy chain with an amino acid sequence of SEQ ID NO: 45 and optionally an Fc polypeptide with an amino acid sequence of SEQ ID NO: 303 and a light chain with an amino acid sequence of SEQ ID NO: 46; or a heavy chain with an amino acid sequence of SEQ ID NO: 47 and optionally an Fc polypeptide with an amino acid sequence of SEQ ID NO: 304 and a light chain with an amino acid sequence of SEQ ID NO: 48; or a heavy chain with an amino acid sequence of SEQ ID NO: 49 and optionally an Fc polypeptide with an amino acid sequence of SEQ ID NO: 305; and a light chain with an amino acid sequence of SEQ ID NO: 50; a heavy chain with an amino acid sequence of SEQ ID NO: 39 and optionally an Fc polypeptide with an amino acid sequence of SEQ ID NO: 301 and a light chain with an amino acid sequence of SEQ ID NO: 40; a heavy chain with an amino acid sequence of SEQ ID NO: 362 and optionally an Fc polypeptide with an amino acid sequence of SEQ ID NO: 393 and a light chain with an amino acid sequence of SEQ ID NO: 363; a heavy chain with an amino acid sequence of SEQ ID NO: 331 and optionally an Fc polypeptide with an amino acid sequence of SEQ ID NO: 355 and a light chain with an amino acid sequence of SEQ ID NO: 332; a heavy chain with an amino acid sequence of SEQ ID NO: 333 and optionally an Fc polypeptide with an amino acid sequence of SEQ ID NO: 356 and a light chain with an amino acid sequence of SEQ ID NO: 334; a heavy chain with an amino acid sequence of SEQ ID NO: 335 and optionally an Fc polypeptide with an amino acid sequence of SEQ ID NO: 357 and a light chain with an amino acid sequence of SEQ ID NO: 336; a heavy chain with an amino acid sequence of SEQ ID NO: 337 and optionally an Fc polypeptide with an amino acid sequence of SEQ ID NO: 358 and a light chain with an amino acid sequence of SEQ ID NO: 338; a heavy chain with an amino acid sequence of SEQ ID NO: 339 and a light chain with an amino acid sequence of SEQ ID NO: 340; a heavy chain with an amino acid sequence of SEQ ID NO: 59 and optionally an Fc polypeptide with an amino acid sequence of SEQ ID NO: 309 and a light chain with an amino acid sequence of SEQ ID NO: 60; a heavy chain with an amino acid sequence of SEQ ID NO: 61 and optionally an Fc polypeptide with an amino acid sequence of SEQ ID NO: 310 and a light chain with an amino acid sequence of SEQ ID NO: 62; and a heavy chain with an amino acid sequence of SEQ ID NO: 341 and optionally an Fc polypeptide with an amino acid sequence of SEQ ID NO: 359 and a light chain with an amino acid sequence of SEQ ID NO: 342.

120. The method of paragraph 119, wherein the transgene comprises a nucleotide sequence of SEQ ID NO: 115 encoding the heavy chain and a nucleotide sequence of SEQ ID NO: 116 encoding the light chain; or a nucleotide sequence of SEQ ID NO: 117 encoding the heavy chain and a nucleotide sequence of SEQ ID NO: 118 encoding the light chain; or a nucleotide sequence of SEQ ID NO: 119 encoding the heavy chain and a nucleotide sequence of SEQ ID NO: 120 encoding the light chain; nucleotide sequence of SEQ ID NO: 109 encoding the heavy chain and a nucleotide sequence of SEQ ID NO: 110 encoding the light chain; or a nucleotide sequence of SEQ ID NO: 378 encoding the heavy chain and a nucleotide sequence of SEQ ID NO: 379 encoding the light chain; a nucleotide sequence of SEQ ID NO: 343 encoding the heavy chain and a nucleotide sequence of SEQ ID NO: 344 encoding the light chain; nucleotide sequence of SEQ ID NO: 345 encoding the heavy chain and a nucleotide sequence of SEQ ID NO: 346 encoding the light chain; a nucleotide sequence of SEQ ID NO: 347 encoding the heavy chain and a nucleotide sequence of SEQ ID NO: 348 encoding the light chain; a nucleotide sequence of SEQ ID NO: 349 encoding the heavy chain and a nucleotide sequence of SEQ ID NO: 350 encoding the light chain; nucleotide sequence of SEQ ID NO: 351 encoding the heavy chain and a nucleotide sequence of SEQ ID NO: 352 encoding the light chain; a nucleotide sequence of SEQ ID NO: 129 encoding the heavy chain and a nucleotide sequence of SEQ ID NO: 130 encoding the light chain; a nucleotide sequence of SEQ ID NO: 131 encoding the heavy chain and a nucleotide sequence of SEQ ID NO: 132 encoding the light chain; or nucleotide sequence of SEQ ID NO: 341 encoding the heavy chain and a nucleotide sequence of SEQ ID NO: 342 encoding the light chain.

121. The method of any of paragraphs 115 to 118, wherein the antibody or antigen-binding fragment thereof is a hyperglycosylated mutant or wherein the Fc polypeptide of the mAb is glycosylated or aglycosylated.

122. The method of any of paragraphs 115 to 121 wherein the mAb or antigen-binding fragment thereof contains an alpha 2,6-sialylated glycan.

123. The method of any of paragraphs 115 to 122 wherein the mAb or antigen-binding fragment thereof is glycosylated but does not contain detectable NeuGc or α-Gal.

124. The method of any of paragraphs 115 to 123 wherein the mAb or antigen-binding fragment thereof contains a tyrosine sulfation.

125. The method of any of paragraphs 116 to 124 wherein the recombinant expression vector is AAV2.7m8, AAV8, or AAV9.

126. The method of any of paragraphs 116 to 125 in which production of the HuPTM form of the mAb or antigen-binding fragment thereof is confirmed by transducing human retina cells in culture with said recombinant nucleotide expression vector and expressing said mAb or antigen-binding fragment thereof.

127. A method of treating multiple sclerosis in a human subject in need thereof, comprising delivering to the cerebrospinal fluid (CSF) of said human subject, a therapeutically effective amount of a substantially full-length or full-length anti-repulsive guidance molecule-A (anti-RGMa) mAb or antigen-binding fragment thereof, expressed from a transgene and produced by human CNS cells.

128. A method of treating multiple sclerosis in a human subject in need thereof, comprising:
administering to the CNS of said human subject, a therapeutically effective amount of a recombinant nucleotide expression vector comprising a transgene encoding a substantially full-length or full-length anti-repulsive guidance molecule-A (anti-RGMa) mAb or antigen-binding fragment thereof, operably linked to one or more regulatory sequences that control expression of the transgene in human CNS cells, so that a depot is formed that released a HuPTM form of said mAb or antigen-binding fragment thereof.

129. The method of paragraphs 127 or 128 wherein the anti-RGMa mAb is elezanumab.

130. The method of any of paragraphs 127 to 129 wherein the antigen-binding fragment is a Fab, a F(ab′)₂, or an scFv.

131. The method of any of paragraphs 127 to 130, wherein the full-length mAb or the antigen-binding fragment comprises a heavy chain with an amino acid sequence of SEQ ID NO: 51 and optionally an Fc polypeptide with an amino acid sequence of SEQ ID NO: 306 and a light chain with an amino acid sequence of SEQ ID NO: 52.

132. The method of paragraph 131, wherein the transgene comprises a nucleotide sequence of SEQ ID NO: 121 encoding the heavy chain and a nucleotide sequence of SEQ ID NO: 122 encoding the light chain.

133. The method of any of paragraphs 127 to 131, wherein the mAb or antigen-binding fragment thereof is a hyperglycosylated mutant or wherein the Fc polypeptide of the mAb is glycosylated or aglycosylated.

134. The method of any of paragraphs 127 to 133 wherein the mAb or antigen-binding fragment thereof contains an alpha 2,6-sialylated glycan.

135. The method of any of paragraphs 127 to 134 wherein the mAb or antigen-binding fragment thereof is glycosylated but does not contain detectable NeuGc or α-Gal.

136. The method of any of paragraphs 127 to 135 wherein the mAb or antigen-binding fragment thereof contains a tyrosine sulfation.

137. The method of any of paragraphs 128 to 136 wherein the recombinant expression vector is AAV9.

138. The method of any of paragraphs 128 to 136 in which production of the HuPTM form of said mAb or antigen-binding fragment thereof is confirmed by transducing human CNS cells in culture with said recombinant nucleotide expression vector and expressing said mAb or antigen-binding fragment thereof.

139. A method of treating amyloidosis (ATTR), familial amyloid cardiomyopathy (FAC), or familial amyloid polyneuropathy (FAP) in a human subject in need thereof, comprising delivering to circulation of said human subject, a therapeutically effective amount of a substantially full-length or full-length anti-transthyretin (anti-TTR) mAb or antigen-binding fragment thereof, expressed from a transgene and produced by human liver cells or human muscle cells.

140. A method of treating asthma in a human subject in need thereof, comprising:
administering to the liver or muscle of said human subject a therapeutically effective amount of a recombinant nucleotide expression vector comprising a transgene encoding a substantially full-length or full-length anti-transthyretin (anti-TTR) mAb or an antigen-binding fragment thereof, operably linked to one or more regulatory sequences that control expression of the transgene in human liver cells or human muscle cells, so that a depot is formed that releases a HuPTM form of said mAb or antigen-binding fragment thereof.

141. The method of paragraphs 139 or 140 wherein the anti-TTR mAb is NI-301 or PRX-004.

142. The method of any of paragraphs 139 to 141 wherein the antigen-binding fragment is a Fab, a F(ab′)₂, or an scFv.

143. The method of any of paragraphs 139 to 142, wherein the full-length mAb or the antigen-binding fragment comprises a heavy chain with an amino acid sequence of SEQ ID NO: 53 and optionally an Fc polypeptide of an IgG1 isotype (e.g., an amino acid sequence of SEQ ID NO: 283) and a light chain with an amino acid sequence of SEQ ID NO: 54; or a heavy chain with an amino acid sequence of SEQ ID NO: 55 and optionally an Fc polypeptide with an amino acid sequence of SEQ ID NO: 307 and a light chain with an amino acid sequence of SEQ ID NO: 56.

144. The method of paragraph 143, wherein the transgene comprises a nucleotide sequence of SEQ ID NO: 123 encoding the heavy chain and a nucleotide sequence of SEQ ID NO: 124 encoding the light chain; or a nucleotide sequence of SEQ ID NO: 125 encoding the heavy chain and a nucleotide sequence of SEQ ID NO: 126 encoding the light chain.

145. The method of any of paragraphs 139 to 143, wherein the antibody or antigen-binding fragment thereof is a hyperglycosylated mutant or wherein the Fc polypeptide of the mAb is glycosylated or aglycosylated.

146. The method of any of paragraphs 139 to 145 wherein the mAb or antigen-binding fragment thereof contains an alpha2,6-sialylated glycan.

147. The method of any of paragraphs 139 to 146 wherein the mAb or antigen-binding fragment thereof is glycosylated but does not contain detectable NeuGc or α-Gal.

148. The method of any of paragraphs 139 to 147 wherein the mAb or antigen-binding fragment thereof contains a tyrosine sulfation.

149. The method of any of paragraphs 140 to 148 wherein the recombinant expression vector is AAV8 or AAV9.

150. The method of any of paragraphs 140 to 149 in which production of said HuPTM form of said mAb or antigen-binding fragment thereof is confirmed by transducing human liver cells or human muscle cells in culture with said recombinant nucleotide expression vector and expressing said mAb or antigen-binding fragment thereof.

151. A method of treating fibrotic disorders including pulmonary fibrosis, cystic fibrosis (CF), idiopathic pulmonary fibrosis (IPF), liver cirrhosis, atrial fibrosis, endomyocardial fibrosis, old myocardial infarction, arthrofibrosis, Crohn's disease, mediastinal fibrosis, myelofibrosis (MF), nephrogenic systemic fibrosis (NSF), progressive massive fibrosis (PMF), and retroperitoneal fibrosis (RPF) in a human subject in need thereof, comprising delivering to the circulation of said human subject, a therapeutically effective amount of a substantially full-length or full-length anti-connective tissue growth factor (anti-CTGF) mAb or antigen-binding fragment thereof, expressed from a transgene and produced by human liver cells or human muscle cells.

152. A method of treating fibrotic disorders including pulmonary fibrosis, cystic fibrosis (CF), idiopathic pulmonary fibrosis (IPF), liver cirrhosis, atrial fibrosis, endomyocardial fibrosis, old myocardial infarction, arthrofibrosis, Crohn's disease, mediastinal fibrosis, myelofibrosis (MF), nephrogenic systemic fibrosis (NSF), progressive massive fibrosis (PMF), and retroperitoneal fibrosis (RPF) in a human subject in need thereof, comprising:
administering to the liver or muscle of said human subject a therapeutically effective amount of a recombinant nucleotide expression vector comprising a transgene encoding a substantially full-length or full-length anti-connective tissue growth factor (anti-CTGF) mAb, or an antigen-binding fragment thereof, operably linked to one or more regulatory sequences that control expression of the transgene in human liver cells or human muscle cells, so that a depot is formed that releases a HuPTM form of the mAb or antigen-binding fragment thereof.

153. The method of paragraph 151 or 152 wherein the anti-CTGF mAb is pamrevlumab.

154. The method of any of paragraphs 151 to 153 wherein the antigen-binding fragment is a Fab, a F(ab′)₂, or an scFv.

155. The method of any of paragraphs 151 to 154, wherein the full-length mAb or the antigen-binding fragment comprises a heavy chain with an amino acid sequence of SEQ ID NO: 57 and optionally an Fc polypeptide with an amino acid sequence of SEQ ID NO: 308 and a light chain with an amino acid sequence of SEQ ID NO: 58.

156. The method of paragraph 155, wherein the transgene comprises a nucleotide sequence of SEQ ID NO: 127 encoding the heavy chain and a nucleotide sequence of SEQ ID NO: 128 encoding the light chain.

157. The method of any of paragraphs 151 to 155, wherein the mAb or antigen-binding fragment thereof is a hyperglycosylated mutant or wherein the Fc polypeptide of the mAb is glycosylated or aglycosylated.

158. The method of any of paragraphs 151 to 157 wherein the mAb or antigen-binding fragment thereof contains an alpha2,6-sialylated glycan.

159. The method of any of paragraphs 151 to 158 wherein the mAb or antigen-binding fragment thereof is glycosylated but does not contain detectable NeuGc or α-Gal.

160. The method of any of paragraphs 151 to 159 wherein the mAb or antigen-binding fragment thereof contains a tyrosine sulfation.

161. The method of any of paragraphs 152 to 160 wherein the recombinant expression vector is AAV8 or AAV9.

162. The method of any of paragraphs 152 to 161 in which production of said HuPTM form of the mAb or antigen-binding fragment thereof is confirmed by transducing human liver cells or human muscle cells in culture with said recombinant nucleotide expression vector and expressing said mAb or antigen-binding fragment thereof.

163. A method of treating non-infectious uveitis, neuromyelitis optica (NMO), diabetic retinopathy (DR) or diabetic macular edema (DME) in a human subject in need thereof, comprising delivering to the retina of said human subject, a therapeutically effective amount of a substantially full-length or full-length mAb of an anti-interleukin-6 receptor (anti-IL6R) mAb, anti-interleukin-6 (IL6) mAb, or anti-cluster of differentiation 19 (anti-CD19) mAb, or antigen-binding fragment thereof, expressed from a transgene and produced by human retina cells.

164. A method of treating non-infectious uveitis, neuromyelitis optica (NMO), diabetic retinopathy (DR) or diabetic macular edema (DME) in a human subject in need thereof, comprising:
administering to the retina of said human subject a therapeutically effective amount of a recombinant nucleotide expression vector comprising a transgene encoding a substantially full-length or full-length mAb of an anti-interleukin-6 receptor (anti-IL6R) mAb, anti-interleukin-6 (IL6) mAb, or anti-cluster of differentiation 19 (anti-CD19) mAb, or an antigen-binding fragment thereof, operably linked to one or more regulatory sequences that control expression of the transgene in human retina cells, so that a depot is formed that releases a HuPTM form of the mAb or antigen-binding fragment thereof.

165. The method of paragraph 163 or 164 wherein the anti-IL6R is satralizumab, sarilumab, or tocilizumab, or the anti-IL6 mAb is siltuximab, clazakizumab, sirukumab, olokizumab, or gerilimzumab, or the anti-CD19 mAb is inebilizumab.

166. The method of any of paragraphs 163 to 165 wherein the antigen-binding fragment is a Fab, a F(ab′)₂, or an scFv.

167. The method of any of paragraphs 163 to 166, wherein the full-length mAb or the antigen-binding fragment comprises a heavy chain with an amino acid sequence of SEQ ID NO: 59 and optionally an Fc polypeptide with an amino acid sequence of SEQ ID NO: 309 and a light chain with an amino acid sequence of SEQ ID NO: 60; or a heavy chain with an amino acid sequence of SEQ ID NO: 61 and optionally an Fc polypeptide with an amino acid sequence of SEQ ID NO: 310 and a light chain with an amino acid sequence of SEQ ID NO: 62; or a heavy chain with an amino acid sequence of SEQ ID NO: 341 and optionally an Fc polypeptide with an amino acid sequence of SEQ ID NO: 359 and a light chain with an amino acid sequence of SEQ ID NO: 342; or a heavy chain with an amino acid sequence of SEQ ID NO: 331 and optionally an Fc polypeptide with an amino acid sequence of SEQ ID NO: 355 and a light chain with an amino acid sequence of SEQ ID NO: 332; or a heavy chain with an amino acid sequence of SEQ ID NO: 333 and optionally an Fc polypeptide with an amino acid sequence of SEQ ID NO: 356 and a light chain with an amino acid sequence of SEQ ID NO: 334; or a heavy chain with an amino acid sequence of SEQ ID NO: 335 and optionally an Fc polypeptide with an amino acid sequence of SEQ ID NO: 357 and a light chain with an amino acid sequence of SEQ ID NO: 336; or a heavy chain with an amino acid sequence of SEQ ID NO: 337 and optionally an Fc polypeptide with an amino acid sequence of SEQ ID NO: 358 and a light chain with an amino acid sequence of SEQ ID NO: 338; or a heavy chain with an amino acid sequence of SEQ ID NO: 339 and optionally an Fc polypeptide with an IgG1 amino acid sequence of SEQ ID NO: 283 and a light chain with an amino acid sequence of SEQ ID NO: 340; or a heavy chain with an amino acid sequence of SEQ ID NO: 341 and optionally an Fc polypeptide with an amino acid sequence of SEQ ID NO: 359 and a light chain with an amino acid sequence of SEQ ID NO: 342; a heavy chain with an amino acid sequence of SEQ ID NO: 63 and optionally an Fc polypeptide with an amino acid sequence of SEQ ID NO: 311 and a light chain with an amino acid sequence of SEQ ID NO: 64.

168. The method of paragraph 167, wherein the transgene comprises a nucleotide sequence of SEQ ID NO: 129 encoding the heavy chain and a nucleotide sequence of SEQ ID NO: 130 encoding the light chain; or a nucleotide sequence of SEQ ID NO: 131 encoding the heavy chain and a nucleotide sequence of SEQ ID NO: 132 encoding the light chain; or a nucleotide sequence of SEQ ID NO: 343 encoding the heavy chain and a nucleotide sequence of SEQ ID NO: 344 encoding the light chain; or a nucleotide sequence of SEQ ID NO: 345 encoding the heavy chain and a nucleotide sequence of SEQ ID NO: 346 encoding the light chain; or a nucleotide sequence of SEQ ID NO: 347 encoding the heavy chain and a nucleotide sequence of SEQ ID NO: 348 encoding the light chain; or a nucleotide sequence of SEQ ID NO: 349 encoding the heavy chain and a nucleotide sequence of SEQ ID NO: 350 encoding the light chain; or a nucleotide sequence of SEQ ID NO: 351 encoding the heavy chain and a nucleotide sequence of SEQ ID NO: 352 encoding the light chain; or a nucleotide sequence of SEQ ID NO: 353 encoding the heavy chain and a nucleotide sequence of SEQ ID NO: 354 encoding the light chain; or a nucleotide sequence of SEQ ID NO: 133 encoding the heavy chain and a nucleotide sequence of SEQ ID NO: 134 encoding the light chain.

169. The method of any of paragraphs 163 to 167, wherein the mAb or antigen-binding fragment thereof is a hyperglycosylated mutant or wherein the Fc polypeptide of the mAb is glycosylated or aglycosylated.

170. The method of any of paragraphs 163 to 168 wherein the mAb or antigen-binding fragment thereof contains an alpha 2,6-sialylated glycan.

171. The method of any of paragraphs 163 to 169 wherein the mAb or antigen-binding fragment thereof is glycosylated but does not contain detectable NeuGc or α-Gal.

172. The method of any of paragraphs 163 to 170 wherein the mAb or antigen-binding fragment thereof contains a tyrosine sulfation.

173. The method of any of paragraphs 164 to 171 wherein the recombinant expression vector is AAV8, AAV2.7m8 or AAV9.

174. The method of any of paragraphs 164 to 172 in which production of said HuPTM form of said mAb or antigen-binding fragment thereof is confirmed by transducing human retina cells in culture with said recombinant nucleotide expression vector and expressing said mAb or antigen-binding fragment thereof.

175. A method of treating inflammatory bowel disease (IBD) including UC and CD in a human subject in need thereof, comprising delivering to the circulation of said human subject, a therapeutically effective amount of a substantially full-length or full-length anti-integrin 137 subunit (anti-ITGB7) mAb or antigen-binding fragment thereof, expressed from a transgene and produced by human liver cells or human muscle cells.

176. A method of treating inflammatory bowel disease (IBD) including UC and CD in a human subject in need thereof, comprising:
administering to the liver or muscle of said human subject a therapeutically effective amount of a recombinant nucleotide expression vector comprising a transgene encoding a substantially full-length or full-length anti-integrin β7 subunit (anti-ITGB7) mAb or an antigen-binding fragment thereof, operably linked to one or more regulatory sequences that control expression of the transgene in human liver cells or human muscle cells, so that a depot is formed that releases a HuPTM form of said mAb or antigen-binding fragment thereof.

177. The method of paragraph 175 or 176 wherein the anti-ITGB7 mAb is etrolizumab.

178. The method of any of paragraphs 175 to 177 wherein the antigen-binding fragment is a Fab, a F(ab′)₂, or an scFv.

179. The method of any of paragraphs 175 to 178, wherein the full-length mAb or the antigen-binding fragment comprises a heavy chain with an amino acid sequence of SEQ ID NO: 65 and optionally an Fc polypeptide with an amino acid sequence of SEQ ID NO: 312 and a light chain with an amino acid sequence of SEQ ID NO: 66.

180. The method of paragraph 179, wherein the transgene comprises a nucleotide sequence of SEQ ID NO: 135 encoding the heavy chain and a nucleotide sequence of SEQ ID NO: 136 encoding the light chain.

181. The method of any of paragraphs 175 to 179, wherein the mAb or antigen-binding fragment thereof is a hyperglycosylated mutant or wherein the Fc polypeptide of the mAb is glycosylated or aglycosylated.

182. The method of any of paragraphs 175 to 181 wherein the mAb or antigen-binding fragment thereof contains an alpha 2,6-sialylated glycan.

183. The method of any of paragraphs 175 to 182 wherein the mAb or antigen-binding fragment thereof is glycosylated but does not contain detectable NeuGc or α-Gal.

184. The method of any of paragraphs 175 to 183 wherein the mAb or antigen-binding fragment thereof contains a tyrosine sulfation.

185. The method of any of paragraphs 176 to 184 wherein the recombinant expression vector is AAV8 or AAV9.

186. The method of any of paragraphs 176 to 185 in which production of said HuPTM form of said mAb or antigen-binding fragment thereof is confirmed by transducing human liver cells or human muscle cells in culture with said recombinant nucleotide expression vector and expressing said mAb or antigen-binding fragment thereof.

187. A method of treating systemic osteoporosis or abnormal bone loss or weakness (e.g., treating giant cell tumor of bone, treating treatment-induced bone loss, slowing the loss of (or increasing) bone mass in breast and prostate cancer patients, preventing skeletal-related events due to bone metastasis or for decreasing bone resorption and turnover in a human subject in need thereof, comprising delivering to the circulation of said human subject, a therapeutically effective amount of a substantially full-length or full-length anti-sclerostin (anti-SOST) mAb or antigen-binding fragment thereof, expressed from a transgene and produced by human liver cells or human muscle cells.

188. A method of treating osteoporosis or abnormal bone loss or weakness (e.g., treating giant cell tumor of bone, treating treatment-induced bone loss, slowing the loss of (or increasing) bone mass in breast and prostate cancer patients, preventing skeletal-related events due to bone metastasis or for decreasing bone resorption and turnover in a human subject in need thereof, comprising:
administering to the liver or muscle of said subject a therapeutically effective amount of a recombinant nucleotide expression vector comprising a transgene encoding a substantially full-length or full-length anti-sclerostin (anti-SOST) mAb or an antigen-binding fragment thereof, operably linked to one or more regulatory sequences that control expression of the transgene in human liver cells or in human muscle cells, so that a depot is formed that releases a HuPTM form of said mAb or antigen-binding fragment thereof.

189. The method of paragraph 187 or 188 wherein the anti-SOST mAb is romosozumab.

190. The method of any of paragraphs 187 to 189 wherein the antigen-binding fragment is a Fab, a F(ab′)₂, or an scFv.

191. The method of any of paragraphs 187 to 190, wherein the full-length mAb or the antigen-binding fragment comprises a heavy chain with an amino acid sequence of SEQ ID NO: 67 and optionally an Fc polypeptide with an amino acid sequence of SEQ ID NO: 313 and a light chain with an amino acid sequence of SEQ ID NO: 68.

192. The method of paragraph 191, wherein the transgene comprises a nucleotide sequence of SEQ ID NO: 137 encoding the heavy chain and a nucleotide sequence of SEQ ID NO: 138 encoding the light chain.

193. The method of any of paragraphs 187 to 191, wherein the mAb or antigen-binding fragment thereof is a hyperglycosylated mutant or wherein the Fc polypeptide of the mAb is glycosylated or aglycosylated.

194. The method of any of paragraphs 187 to 193 wherein the mAb or antigen-binding fragment thereof contains an alpha2,6-sialylated glycan.

195. The method of any of paragraphs 187 to 194 wherein the mAb or antigen-binding fragment thereof is glycosylated but does not contain detectable NeuGc or α-Gal.

196. The method of any of paragraphs 187 to 195 wherein the mAb or antigen-binding fragment thereof contains a tyrosine sulfation.

197. The method of any of paragraphs 188 to 196 wherein the recombinant expression vector is AAV8 or AAV9.

198. The method of any of paragraphs 188 to 196 in which production of said HuPTM form of said mAb or antigen-binding fragment thereof is confirmed by transducing human liver cells or human muscle cells in culture with said recombinant nucleotide expression vector and expressing said mAb or antigen-binding fragment thereof.

199. A method of treating angioedema in a human subject in need thereof, comprising delivering to the circulation of said human subject, a therapeutically effective amount of a substantially full-length or full-length anti-kallikrein (anti-pKal) mAb or antigen-binding fragment thereof, expressed from a transgene and produced by human muscle cells or human liver cells.

200. A method of treating angioedema in a human subject in need thereof, comprising:
administering to the muscle or liver of said subject, a therapeutically effective amount of a recombinant nucleotide expression vector comprising a transgene encoding a substantially full-length or full-length anti-kallikrein (anti-pKal) mAb or an antigen-binding fragment thereof, operably linked to one or more regulatory sequences that control expression of the transgene in human muscle cells or human liver cells, so that a depot is formed that releases a HuPTM form of said mAb or antigen-binding fragment thereof.

201. The method of paragraph 199 or 200 wherein the anti-pKal mAb is lanadelumab.

202. The method of any of paragraphs 199 to 201 wherein the antigen-binding fragment is a Fab, a F(ab′)₂, or an scFv.

203. The method of any of paragraphs 199 to 202, wherein the full-length mAb or the antigen-binding fragment comprises a heavy chain with an amino acid sequence of SEQ ID NO: 69 and optionally an Fc polypeptide with an amino acid sequence of SEQ ID NO: 314 and a light chain with an amino acid sequence of SEQ ID NO: 70.

204. The method of paragraph 203, wherein the transgene comprises a nucleotide sequence of SEQ ID NO: 139 encoding the heavy chain and a nucleotide sequence of SEQ ID NO: 140 encoding the light chain.

205. The method of any of paragraphs 199 to 203, wherein the mAb or antigen-binding fragment thereof is a hyperglycosylated mutant or wherein the Fc polypeptide of the mAb is glycosylated or aglycosylated.

206. The method of any of paragraphs 199 to 205 wherein the mAb or antigen-binding fragment thereof contains an alpha 2,6-sialylated glycan.

207. The method of any of paragraphs 199 to 206 wherein the mAb or antigen-binding fragment thereof is glycosylated but does not contain detectable NeuGc or α-Gal.

208. The method of any of paragraphs 199 to 207 wherein the mAb or antigen-binding fragment thereof contains a tyrosine sulfation.

209. The method of any of paragraphs 200 to 208 wherein the recombinant expression vector is AAV8 or AAV9.

210. The method of any of paragraphs 200 to 209 in which production of said HuPTM form of said mAb or antigen-binding fragment thereof is confirmed by transducing human liver cells or human muscle cells in culture with said recombinant nucleotide expression vector and expressing said mAb or antigen-binding fragment thereof.

### Method of Manufacture

211. A method of producing recombinant AAVs comprising:
(a) culturing a host cell containing:
   (i) an artificial genome comprising a cis expression cassette flanked by AAV ITRs, wherein the cis expression cassette comprises a transgene encoding a therapeutic antibody operably linked to expression control elements that will control expression of the transgene in human cells;
   (ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans;
   (iii) sufficient adenovirus helper functions to permit replication and packaging of the artificial genome by the AAV capsid protein; and
(b) recovering recombinant AAV encapsidating the artificial genome from the cell culture.

212. The method of paragraph 211, wherein the transgene encodes a substantially full-length or full-length mAb or antigen binding fragment that comprises the heavy and light chain variable domains of solanezumab, lecanemab, GSK933776, AL-001, ABBV-8E12, UCB-0107, NI-105 (BIIB076), VX15/2503, prasinezumab, NI-202 (BIIB054), MED-1341, NI-2041.10D12, NI-204.12G7, eptinezumab, fremanezumab, galcanezumab, or elezanumab.

213. The method of paragraph 212, wherein the AAV capsid protein is an AAV9, AAVrh10, AAVrh20, AAVrh39, or AAVcy5 capsid protein.

214. The method of paragraph 211, wherein the transgene encodes a substantially full-length or full-length mAb or antigen binding fragment that comprises the heavy and light chain variable domains of sevacizumab, LKA-651 (NSV2), LKA-651 (NSV3), GSK933776, solanezumab, lecanemab, ascrinvacumab, tesidolumab, ravulizumab, carotuximab, ANX-007, lanadelumab, adalimumab, infliximab, golimumab, satralizumab, sarilumab, tocilizumab, siltuximab, clazakizumab, sirukumab, olokizumab, gerilimzumab, or inebilizumab.

215. The method of paragraph 214, wherein the AAV capsid protein is an AAV2.7m8, AAV8, or AAV9 capsid protein.

216. The method of paragraph 211, wherein the transgene encodes a substantially full-length or full-length mAb or antigen binding fragment that comprises the heavy and light chain variable domains of NI-301, PRX-004, pamrevlumab, etrolizumab, romosozumab, or lanadelumab.

217. The method of paragraph 216, wherein the AAV capsid protein is an AAV8, AAV9, or AAVrh10 capsid protein.

218. The method of paragraph 211, wherein the transgene encodes a substantially full-length or full-length mAb.

### Autoimmune, Respiratory, and Allergic Disease

Compositions of Matter

219. A pharmaceutical composition for treating atopic dermatitis in a human subject in need thereof, comprising an AAV vector comprising:
(a) a viral capsid that is at least 95% identical to the amino acid sequence of an AAV8 capsid (SEQ ID NO: 143), an AAV9 capsid (SEQ ID NO: 144), or an AAVrh10 capsid (SEQ ID NO: 145); and
(b) an artificial genome comprising an expression cassette flanked by AAV ITRs wherein the expression cassette comprises a transgene encoding an anti-IL13 mAb or anti-IL31RA, or an antigen-binding fragment thereof, operably linked to one or more regulatory sequences that control expression of the transgene in human liver cells or human muscle cells;
wherein said AAV vector is formulated for intravenous administration to the liver or muscle of said subject.

220. The pharmaceutical composition of paragraph 219 wherein the anti-IL13 or the IL31RA is tralokinumab or nemolizumab.

221. The pharmaceutical composition of paragraphs 219 or 220, wherein the antigen-binding fragment is a Fab, a F(ab′)₂, or an scFv.

222. The pharmaceutical composition of any of paragraphs 219 to 221, wherein the antigen-binding fragment comprises a heavy chain with an amino acid sequence of SEQ ID NO: 368 and optionally an Fc polypeptide of an amino acid sequence of SEQ ID NO: 396 and a light chain with an amino acid sequence of SEQ ID NO: 369; or the antigen-binding fragment comprises a heavy chain with an amino acid sequence of SEQ ID NO: 370 and optionally an Fc polypeptide of an amino acid sequence of SEQ ID NO: 397 and a light chain with an amino acid sequence of SEQ ID NO: 371.

223. The pharmaceutical composition of paragraph 222, wherein the transgene comprises a nucleotide sequence of SEQ ID NO: 384 encoding the heavy chain and a nucleotide sequence of SEQ ID NO: 385 encoding the light chain; or the transgene comprises a nucleotide sequence of SEQ ID NO: 386 encoding the heavy chain and a nucleotide sequence of SEQ ID NO: 387 encoding the light chain.

224. The pharmaceutical composition of any of paragraphs 219 to 221, wherein the antibody or antigen-binding fragment thereof is a hyperglycosylated mutant.

225. The pharmaceutical composition of any of paragraphs 219 to 224, wherein the transgene encodes a signal sequence at the N-terminus of the heavy chain and the light chain of said antigen-binding fragment that directs secretion and post translational modification in said human liver cells or human muscle cells.

226. The pharmaceutical composition of paragraph 225, wherein said signal sequence is selected from the signal sequences in Table 2 or 3.

227. The pharmaceutical composition of any of paragraphs 219 to 226, wherein the AAV capsid is AAV8.

228. A pharmaceutical composition for treating eosinophilic asthma in a human subject in need thereof, comprising an AAV vector comprising:
(a) a viral capsid that is at least 95% identical to the amino acid sequence of an AAV8 capsid (SEQ ID NO: 143), an AAV9 capsid (SEQ ID NO: 144), or an AAVrh10 capsid (SEQ ID NO: 145); and
(b) an artificial genome comprising an expression cassette flanked by AAV ITRs wherein the expression cassette comprises a transgene encoding an anti-IL5R mAb or anti-IgE mAb, or an antigen-binding fragment thereof, operably linked to one or more regulatory sequences that control expression of the transgene in human liver cells or human muscle cells;
wherein said AAV vector is formulated for intravenous administration to the liver or muscle of said subject.

229. The pharmaceutical composition of paragraph 228 wherein the anti-IL5R or anti-IgE mAb is reslizumab or omalizumab.

230. The pharmaceutical composition of paragraphs 228 or 229, wherein the antigen-binding fragment is a Fab, a F(ab′)₂, or an scFv.

231. The pharmaceutical composition of any of paragraphs 228 to 230, wherein the antigen-binding fragment comprises a heavy chain with an amino acid sequence of SEQ ID NO: 364 and optionally an Fc polypeptide of an amino acid sequence of SEQ ID NO: 394 and a light chain with an amino acid sequence of SEQ ID NO: 365; or a heavy chain with an amino acid sequence of SEQ ID NO: 372 and optionally an Fc polypeptide of an amino acid sequence of SEQ ID NO: 398 and a light chain with an amino acid sequence of SEQ ID NO: 373.

232. The pharmaceutical composition of paragraph 231, wherein the transgene comprises a nucleotide sequence of SEQ ID NO: 380 encoding the heavy chain and a nucleotide sequence of SEQ ID NO: 381 encoding the light chain; or a nucleotide sequence of SEQ ID NO: 388 encoding the heavy chain and a nucleotide sequence of SEQ ID NO: 389 encoding the light chain.

233. The pharmaceutical composition of any of paragraphs 228 to 231, wherein the antibody or antigen-binding fragment thereof is a hyperglycosylated mutant.

234. The pharmaceutical composition of any of paragraphs 228 to 233, wherein the transgene encodes a signal sequence at the N-terminus of the heavy chain and the light chain of said antigen-binding fragment that directs secretion and post translational modification in said human liver cells or human muscle cells.

235. The pharmaceutical composition of paragraph 234, wherein said signal sequence is selected from the signal sequences in Table 2 or 3.

236. The pharmaceutical composition of any of paragraphs 228 to 235, wherein the AAV capsid is AAV8.

237. A pharmaceutical composition for treating asthma or chronic obstructive pulmonary disease (COPD) in a human subject in need thereof, comprising an AAV vector comprising:
(a) a viral capsid that is at least 95% identical to the amino acid sequence of an AAV8 capsid (SEQ ID NO: 143), an AAV9 capsid (SEQ ID NO: 144), or an AAVrh10 capsid (SEQ ID NO: 145); and
(b) an artificial genome comprising an expression cassette flanked by AAV ITRs wherein the expression cassette comprises a transgene encoding an anti-IL5, anti-IL-5R, anti-IgE, or anti-TSLP mAb, or an antigen-binding fragment thereof, operably linked to one or more regulatory sequences that control expression of the transgene in human liver cells or human muscle cells;
wherein said AAV vector is formulated for intravenous administration to the liver or muscle of said subject.

238. The pharmaceutical composition of paragraph 237 wherein the anti-IL-5, anti-IL5R, anti-IgE, or anti-TSLP mAb is benralizumab, reslizumab, omalizumab, or tezepelumab.

239. The pharmaceutical composition of paragraphs 237 or 238, wherein the antigen-binding fragment is a Fab, a F(ab′)₂, or an scFv.

240. The pharmaceutical composition of any of paragraphs 237 to 239, wherein the antigen-binding fragment comprises a heavy chain with an amino acid sequence of SEQ ID NO: 364 and optionally an Fc polypeptide of an amino acid sequence of SEQ ID NO: 394 and a light chain with an amino acid sequence of SEQ ID NO: 365; a heavy chain with an amino acid sequence of SEQ ID NO: 366 and optionally an Fc polypeptide of an amino acid sequence of SEQ ID NO: 395 and a light chain with an amino acid sequence of SEQ ID NO: 367; a heavy chain with an amino acid sequence of SEQ ID NO: 372 and a light chain with an amino acid sequence of SEQ ID NO: 373; or a heavy chain with an amino acid sequence of SEQ ID NO: 374 and optionally an IgG2 Fc polypeptide of an amino acid sequence of SEQ ID NO: 284 and a light chain with an amino acid sequence of SEQ ID NO: 375

241. The pharmaceutical composition of paragraph 240, wherein the transgene comprises a nucleotide sequence of SEQ ID NO: 380 encoding the heavy chain and a nucleotide sequence of SEQ ID NO: 381 encoding the light chain; a nucleotide sequence of SEQ ID NO: 382 encoding the heavy chain and a nucleotide sequence of SEQ ID NO: 383 encoding the light chain; a nucleotide sequence of SEQ ID NO: 388 encoding the heavy chain and a nucleotide sequence of SEQ ID NO: 389 encoding the light chain; a nucleotide sequence of SEQ ID NO: 390 encoding the heavy chain and a nucleotide sequence of SEQ ID NO: 391 encoding the light chain.

242. The pharmaceutical composition of any of paragraphs 237 to 240, wherein the antibody or antigen-binding fragment thereof is a hyperglycosylated mutant.

243. The pharmaceutical composition of any of paragraphs 237 to 242, wherein the transgene encodes a signal sequence at the N-terminus of the heavy chain and the light chain of said antigen-binding fragment that directs secretion and post translational modification in said human liver cells or human muscle cells.

244. The pharmaceutical composition of paragraph 243, wherein said signal sequence is selected from the signal sequences in Table 2 or 3.

245. The pharmaceutical composition of any of paragraphs 237 to 244, wherein the AAV capsid is AAV8.

246. A pharmaceutical composition for treating chronic idiopathic urticaria in a human subject in need thereof, comprising an AAV vector comprising:
(a) a viral capsid that is at least 95% identical to the amino acid sequence of an AAV8 capsid (SEQ ID NO:143), an AAV9 capsid (SEQ ID NO: 144), or an AAVrh10 capsid (SEQ ID NO: 145); and
(b) an artificial genome comprising an expression cassette flanked by AAV ITRs wherein the expression cassette comprises a transgene encoding an anti-IgE mAb, or an antigen-binding fragment thereof, operably linked to one or more regulatory sequences that control expression of the transgene in human liver cells or human muscle cells;
wherein said AAV vector is formulated for intravenous administration to the liver or muscle of said subject.

247. The pharmaceutical composition of paragraph 246, wherein the anti-IgE mAb is omalizumab.

248. The pharmaceutical composition of paragraphs 246 or 247, wherein the antigen-binding fragment is a Fab, a F(ab′)₂, or an scFv.

249. The pharmaceutical composition of any of paragraphs 246 to 248, wherein the antigen-binding fragment comprises a heavy chain with an amino acid sequence of SEQ ID NO: 372 and optionally an Fc polypeptide of an amino acid sequence of SEQ ID NO: 398 and a light chain with an amino acid sequence of SEQ ID NO: 373.

250. The pharmaceutical composition of paragraph 249, wherein the transgene comprises a nucleotide sequence of SEQ ID NO: 388 encoding the heavy chain and a nucleotide sequence of SEQ ID NO: 389 encoding the light chain.

251. The pharmaceutical composition of any of paragraphs 246 to 249, wherein the antibody or antigen-binding fragment thereof is a hyperglycosylated mutant.

252. The pharmaceutical composition of any of paragraphs 246 to 251, wherein the transgene encodes a signal sequence at the N-terminus of the heavy chain and the light chain of said antigen-binding fragment that directs secretion and post translational modification in said human liver cells or human muscle cells.

253. The pharmaceutical composition of paragraph 252, wherein said signal sequence is selected from the signal sequences in Table 2 or 3.

The pharmaceutical composition of any of paragraphs 246 to 253, wherein the AAV capsid is AAV8.

### Method of Treatment

254. A method of treating atopic dermatitis in a human subject in need thereof, comprising delivering to the circulation of said human subject, a therapeutically effective amount of an anti-IL13 or anti-IL31RA mAb or antigen-binding fragment thereof, produced by human liver cells or human muscle cells.

255. A method of treating atopic dermatitis in a human subject in need thereof, comprising:
administering to the liver or muscle of said subject a therapeutically effective amount of a recombinant nucleotide expression vector comprising a transgene encoding an anti-IL13 or anti-IL31RA mAb, or an antigen-binding fragment thereof, operably linked to one or more regulatory sequences that control expression of the transgene in human liver cells or human muscle cells, so that a depot is formed that releases a HuPTM form of said mAb or antigen-binding fragment thereof.

256. The method of paragraph 254 or 255 wherein the anti-IL13 or anti-IL31RA mAb is tralokinumab or nemolizumab.

257. The method of any of paragraphs 254 to 256 wherein the antigen-binding fragment is a Fab, a F(ab′)₂, or an scFv.

258. The method of any of paragraphs 254 to 257, wherein the antigen-binding fragment comprises a heavy chain with an amino acid sequence of SEQ ID NO: 368 and optionally an Fc polypeptide of an amino acid sequence of SEQ ID NO: 396 and a light chain with an amino acid sequence of SEQ ID NO: 369; or a heavy chain with an amino acid sequence of SEQ ID NO: 370 and optionally an Fc polypeptide of an amino acid sequence of SEQ ID NO: 397 and a light chain with an amino acid sequence of SEQ ID NO: 371.

259. The method of claim 258, wherein the transgene comprises a nucleotide sequence of SEQ ID NO: 384 encoding the heavy chain and a nucleotide sequence of SEQ ID NO: 385 encoding the light chain; or a nucleotide sequence of SEQ ID NO: 386 encoding the heavy chain and a nucleotide sequence of SEQ ID NO: 387 encoding the light chain.

260. The method of any of paragraphs 254 to 258, wherein the mAb or antigen-binding fragment thereof is a hyperglycosylated mutant.

261. The method of any of paragraphs 254 to 260 wherein the mAb or antigen-binding fragment thereof contains an alpha 2,6-sialylated glycan.

262. The method of any of paragraphs 254 to 261 wherein the mAb or antigen-binding fragment thereof is glycosylated but does not contain detectable NeuGc or α-Gal.

263. The method of any of paragraphs 254 to 262 wherein the mAb or antigen-binding fragment thereof contains a tyrosine sulfation.

264. The method of any of paragraphs 254 to 263 wherein the recombinant expression vector is AAV8 or AAV9.

265. The method of any of paragraphs 254 to 264 in which production of said HuPTM form of the mAb or antigen-binding fragment thereof is confirmed by transducing human liver cells or muscle cells in culture with said recombinant nucleotide expression vector and expressing said mAb or antigen-binding fragment thereof.

266. A method of treating eosinophilic asthma in a human subject in need thereof, comprising delivering to the circulation of said human subject, a therapeutically effective amount of an anti-IL5R or anti-IgE mAb or antigen-binding fragment thereof, produced by human liver cells or human muscle cells.

267. A method of treating eosinophilic asthma in a human subject in need thereof, comprising:
administering to the liver or muscle of said subject a therapeutically effective amount of a recombinant nucleotide expression vector comprising a transgene encoding an anti-IL5R or anti-IgE mAb, or an antigen-binding fragment thereof, operably linked to one or more regulatory sequences that control expression of the transgene in human liver cells or human muscle cells, so that a depot is formed that releases a HuPTM form of said mAb or antigen-binding fragment thereof.

268. The method of paragraph 266 or 267 wherein the anti-IL5R or anti-IgE mAb is reslizumab or omalizumab.

269. The method of any of paragraphs 266 to 268 wherein the antigen-binding fragment is a Fab, a F(ab′)₂, or an scFv.

270. The method of any of paragraphs 266 to 269, wherein the antigen-binding fragment comprises a heavy chain with an amino acid sequence of SEQ ID NO: 366 and optionally an Fc polypeptide of an amino acid sequence of SEQ ID NO: 395 and a light chain with an amino acid sequence of SEQ ID NO: 367; or a heavy chain with an amino acid sequence of SEQ ID NO: 372 and optionally an Fc polypeptide of an amino acid sequence of SEQ ID NO: 398 and a light chain with an amino acid sequence of SEQ ID NO: 373.

271. The method of claim 270, wherein the transgene comprises a nucleotide sequence of SEQ ID NO: 382 encoding the heavy chain and a nucleotide sequence of SEQ ID NO: 383 encoding the light chain; or a nucleotide sequence of SEQ ID NO: 388 encoding the heavy chain and a nucleotide sequence of SEQ ID NO: 389 encoding the light chain.

272. The method of any of paragraphs 266 to 270, wherein the mAb or antigen-binding fragment thereof is a hyperglycosylated mutant.

273. The method of any of paragraphs 266 to 272 wherein the mAb or antigen-binding fragment thereof contains an alpha 2,6-sialylated glycan.

274. The method of any of paragraphs 266 to 273 wherein the mAb or antigen-binding fragment thereof is glycosylated but does not contain detectable NeuGc or α-Gal.

275. The method of any of paragraphs 266 to 274 wherein the mAb or antigen-binding fragment thereof contains a tyrosine sulfation.

276. The method of any of paragraphs 266 to 275 wherein the recombinant expression vector is AAV8 or AAV9.

277. The method of any of paragraphs 266 to 276 in which production of said HuPTM form of the mAb or antigen-binding fragment thereof is confirmed by transducing human liver cells or muscle cells in culture with said recombinant nucleotide expression vector and expressing said mAb or antigen-binding fragment thereof.

278. A method of treating asthma or COPD in a human subject in need thereof, comprising delivering to the circulation of said human subject, a therapeutically effective amount of an anti-IL5, anti-IL5R, anti-IgE, or anti-TSLP mAb or antigen-binding fragment thereof, produced by human liver cells or human muscle cells.

279. A method of treating eosinophilic asthma in a human subject in need thereof, comprising:
administering to the liver or muscle of said subject a therapeutically effective amount of a recombinant nucleotide expression vector comprising a transgene encoding an anti-IL5R, anti-IL5, anti-IgE, or anti-TSLP mAb, or an antigen-binding fragment thereof, operably linked to one or more regulatory sequences that control expression of the transgene in human liver cells or human muscle cells, so that a depot is formed that releases a HuPTM form of said mAb or antigen-binding fragment thereof.

280. The method of paragraph 278 or 279 wherein the anti-IL5R, anti-IL5, anti-IgE, or anti-TSLP mAb is benralizumab, reslizumab, omalizumab, or tezepelumab.

281. The method of any of paragraphs 278 to 280 wherein the antigen-binding fragment is a Fab, a F(ab′)₂, or an scFv.

282. The method of any of paragraphs 278 to 281, wherein the antigen-binding fragment comprises a heavy chain with an amino acid sequence of SEQ ID NO: 364 and optionally an Fc polypeptide of an amino acid sequence of SEQ ID NO: 394 and a light chain with an amino acid sequence of SEQ ID NO: 365; or a heavy chain with an amino acid sequence of SEQ ID NO: 366 and optionally an Fc polypeptide of an amino acid sequence of SEQ ID NO: 395 and a light chain with an amino acid sequence of SEQ ID NO: 367; or a heavy chain with an amino acid sequence of SEQ ID NO: 372 and optionally an Fc polypeptide of an amino acid sequence of SEQ ID NO: 398 and a light chain with an amino acid sequence of SEQ ID NO: 373; or a heavy chain with an amino acid sequence of SEQ ID NO: 374 and optionally an IgG2 Fc polypeptide of an amino acid sequence of SEQ ID NO: 284 and a light chain with an amino acid sequence of SEQ ID NO: 375.

283. The method of paragraph 282, wherein the transgene comprises a nucleotide sequence of SEQ ID NO: 380 encoding the heavy chain and a nucleotide sequence of SEQ ID NO: 381 encoding the light chain; or a nucleotide sequence of SEQ ID NO: 383 encoding the heavy chain and a nucleotide sequence of SEQ ID NO: 383 encoding the light chain; or a nucleotide sequence of SEQ ID NO: 388 encoding the heavy chain and a nucleotide sequence of SEQ ID NO: 389 encoding the light chain; or a nucleotide sequence of SEQ ID NO: 390 encoding the heavy chain and a nucleotide sequence of SEQ ID NO: 391 encoding the light chain.

284. The method of any of paragraphs 278 to 283, wherein the mAb or antigen-binding fragment thereof is a hyperglycosylated mutant.

285. The method of any of paragraphs 278 to 284 wherein the mAb or antigen-binding fragment thereof contains an alpha 2,6-sialylated glycan.

286. The method of any of paragraphs 278 to 285 wherein the mAb or antigen-binding fragment thereof is glycosylated but does not contain detectable NeuGc or α-Gal.

287. The method of any of paragraphs 278 to 286 wherein the mAb or antigen-binding fragment thereof contains a tyrosine sulfation.

288. The method of any of paragraphs 278 to 287 wherein the recombinant expression vector is AAV8 or AAV9.

289. The method of any of paragraphs 278 to 288 in which production of said HuPTM form of the mAb or antigen-binding fragment thereof is confirmed by transducing human liver cells or muscle cells in culture with said recombinant nucleotide expression vector and expressing said mAb or antigen-binding fragment thereof.

290. A method of treating chronic idiopathic urticaria in a human subject in need thereof, comprising delivering to the circulation of said human subject, a therapeutically effective amount of an anti-IgE mAb or antigen-binding fragment thereof, produced by human liver cells or human muscle cells.

291. A method of treating eosinophilic asthma in a human subject in need thereof, comprising:
administering to the liver or muscle of said subject a therapeutically effective amount of a recombinant nucleotide expression vector comprising a transgene encoding an anti-IgE mAb, or an antigen-binding fragment thereof, operably linked to one or more regulatory sequences that control expression of the transgene in human liver cells or human muscle cells, so that a depot is formed that releases a HuPTM form of said mAb or antigen-binding fragment thereof.

292. The method of paragraph 290 or 291 wherein the anti-IgE mAb is omalizumab.

293. The method of any of paragraphs 290 to 292 wherein the antigen-binding fragment is a Fab, a F(ab′)₂, or an scFv.

294. The method of any of paragraphs 290 to 293, wherein the antigen-binding fragment comprises a heavy chain with an amino acid sequence of SEQ ID NO: 372 and optionally an Fc polypeptide of an amino acid sequence of SEQ ID NO: 398 and a light chain with an amino acid sequence of SEQ ID NO: 373.

295. The method of paragraph 294, wherein the transgene comprises a nucleotide sequence of SEQ ID NO: 388 encoding the heavy chain and a nucleotide sequence of SEQ ID NO: 389 encoding the light chain.

296. The method of any of paragraphs 290 to 295, wherein the mAb or antigen-binding fragment thereof is a hyperglycosylated mutant.

297. The method of any of paragraphs 290 to 296 wherein the mAb or antigen-binding fragment thereof contains an alpha 2,6-sialylated glycan.

298. The method of any of paragraphs 290 to 297 wherein the mAb or antigen-binding fragment thereof is glycosylated but does not contain detectable NeuGc or α-Gal.

299. The method of any of paragraphs 290 to 298 wherein the mAb or antigen-binding fragment thereof contains a tyrosine sulfation.

300. The method of any of paragraphs 290 to 299 wherein the recombinant expression vector is AAV8 or AAV9.

301. The method of any of paragraphs 290 to 300 in which production of said HuPTM form of the mAb or antigen-binding fragment thereof is confirmed by transducing human liver cells or muscle cells in culture with said recombinant nucleotide expression vector and expressing said mAb or antigen-binding fragment thereof.

### Method of Manufacture

302. The method of paragraph 211, wherein the transgene encodes a substantially full-length or full-length mAb or antigen binding fragment that comprises the heavy and light chain variable domains of benralizumab, reslizumab, tralokinumab, nemolizumab, omalizumab, or tezepelumab.

303. The method of paragraph 302, wherein the AAV capsid protein is an AAV8, AAV9, or AAVrh10 capsid protein.

### Myasthenia Gravis

Compositions of Matter

304. A pharmaceutical composition for treating myasthenia gravis in a human subject in need thereof, comprising an AAV vector comprising:
(a) a viral capsid that is at least 95% identical to the amino acid sequence of an AAV8 capsid (SEQ ID NO: 143), an AAV9 capsid (SEQ ID NO: 144), or an AAVrh10 capsid (SEQ ID NO: 145); and
(b) an artificial genome comprising an expression cassette flanked by AAV ITRs wherein the expression cassette comprises a transgene encoding an anti-C5 mAb, or an antigen-binding fragment thereof, operably linked to one or more regulatory sequences that control expression of the transgene in human liver cells or human muscle cells;
wherein said AAV vector is formulated for intravenous administration to the liver or muscle of said subject.

305. The pharmaceutical composition of paragraph 304 wherein the anti-C5 is ravulizumab.

306. The pharmaceutical composition of paragraphs 304 or 305, wherein the antigen-binding fragment is a Fab, a F(ab′)₂, or an scFv.

307. The pharmaceutical composition of any of paragraphs 304 to 306, wherein the antigen-binding fragment comprises a heavy chain with an amino acid sequence of SEQ ID NO: 362 and optionally an Fc polypeptide of an amino acid sequence of SEQ ID NO: 393 and a light chain with an amino acid sequence of SEQ ID NO: 363.

308. The pharmaceutical composition of paragraph 307, wherein the transgene comprises a nucleotide sequence of SEQ ID NO: 378 encoding the heavy chain and a nucleotide sequence of SEQ ID NO: 379 encoding the light chain.

309. The pharmaceutical composition of any of paragraphs 304 to 308, wherein the antibody or antigen-binding fragment thereof is a hyperglycosylated mutant.

310. The pharmaceutical composition of any of paragraphs 304 to 310, wherein the transgene encodes a signal sequence at the N-terminus of the heavy chain and the light chain of said antigen-binding fragment that directs secretion and post translational modification in said human liver cells or human muscle cells.

311. The pharmaceutical composition of paragraph 310, wherein said signal sequence is selected from the signal sequences in Table 2 or 3.

312. The pharmaceutical composition of any of paragraphs 304 to 311, wherein the AAV capsid is AAV8.

### Method of Treatment

313. A method of treating myasthenia gravis in a human subject in need thereof, comprising delivering to the circulation of said human subject, a therapeutically effective amount of an anti-C5 mAb or antigen-binding fragment thereof, produced by human liver cells or human muscle cells.

314. A method of treating myasthenia gravis in a human subject in need thereof, comprising:
administering to the liver or muscle of said subject a therapeutically effective amount of a recombinant nucleotide expression vector comprising a transgene encoding an anti-C5 mAb, or an antigen-binding fragment thereof, operably linked to one or more regulatory sequences that control expression of the transgene in human liver cells or human muscle cells, so that a depot is formed that releases a HuPTM form of said mAb or antigen-binding fragment thereof.

315. The method of paragraph 313 or 314 wherein the anti-C5 is ravulizumab.

316. The method of any of paragraphs 313 to 315 wherein the antigen-binding fragment is a Fab, a F(ab′)₂, or an scFv.

317. The method of any of paragraphs 313 to 316, wherein the antigen-binding fragment comprises a heavy chain with an amino acid sequence of SEQ ID NO: 362 and optionally an Fc polypeptide of an amino acid sequence of SEQ ID NO: 393 and a light chain with an amino acid sequence of SEQ ID NO: 363.

318. The method of claim 260, wherein the transgene comprises a nucleotide sequence of SEQ ID NO: 378 encoding the heavy chain and a nucleotide sequence of SEQ ID NO: 379 encoding the light chain.

319. The method of any of paragraphs 313 to 318, wherein the mAb or antigen-binding fragment thereof is a hyperglycosylated mutant.

320. The method of any of paragraphs 313 to 319 wherein the mAb or antigen-binding fragment thereof contains an alpha 2,6-sialylated glycan.

321. The method of any of paragraphs 313 to 320 wherein the mAb or antigen-binding fragment thereof is glycosylated but does not contain detectable NeuGc or α-Gal.

322. The method of any of paragraphs 313 to 321 wherein the mAb or antigen-binding fragment thereof contains a tyrosine sulfation.

323. The method of any of paragraphs 313 to 322 wherein the recombinant expression vector is AAV8 or AAV9.

324. The method of any of paragraphs 313 to 323 in which production of said HuPTM form of the mAb or antigen-binding fragment thereof is confirmed by transducing human liver cells or muscle cells in culture with said recombinant nucleotide expression vector and expressing said mAb or antigen-binding fragment thereof.

### Method of Manufacture

325. The method of paragraph 211, wherein the transgene encodes a substantially full-length or full-length mAb or antigen binding fragment that comprises the heavy and light chain variable domains of ravulizumab.

326. The method of paragraph 304, wherein the AAV capsid protein is an AAV8, AAV9, or AAVrh10 capsid protein.

### Compositions and Methods for Inhibiting Immune Response

327. A pharmaceutical composition for reducing, inhibiting or ameliorating a detrimental immune response in a human subject in need thereof, comprising an AAV vector comprising:
(a) a viral capsid that is at least 95% identical to the amino acid sequence of an AAV8 capsid (SEQ ID NO: 143), an AAV9 capsid (SEQ ID NO: 144), an AAVrh10 capsid (SEQ ID NO: 145); and
(b) an artificial genome comprising an expression cassette flanked by AAV ITRs wherein the expression cassette comprises a transgene encoding a substantially full-length or full-length mAb of an anti-interleukin-6 receptor (anti-IL6R) or anti-interleukin-6 (IL6), or an antigen-binding fragment thereof, operably linked to one or more regulatory sequences that control expression of the transgene in human liver cells or muscle cells;
wherein said AAV vector is formulated for subcutaneous, intramuscular, intravenous or pulmonary administration to the subject.

328. The pharmaceutical composition of paragraph 327, wherein the anti-IL6R mAb is satralizumab, sarilumab, or tocilizumab, or the anti-IL6 mAb is siltuximab, clazakizumab, sirukumab, olokizumab, or gerilimzumab.

329. The pharmaceutical composition of paragraphs 327 or 328, wherein the antigen-binding fragment is a Fab, a F(ab′)₂, or an scFv.

330. The pharmaceutical composition of any of paragraphs 327 to 329, wherein the full-length mAb or the antigen-binding fragment comprises a heavy chain with an amino acid sequence of SEQ ID NO: 59 and optionally an Fc polypeptide with an amino acid sequence of SEQ ID NO: 309 and a light chain with an amino acid sequence of SEQ ID NO: 60; or a heavy chain with an amino acid sequence of SEQ ID NO: 61 and optionally an Fc polypeptide with an amino acid sequence of SEQ ID NO: 310 and a light chain with an amino acid sequence of SEQ ID NO: 62; or a heavy chain with an amino acid sequence of SEQ ID NO: 331 and optionally an Fc polypeptide with an amino acid sequence of SEQ ID NO: 355 and a light chain with an amino acid sequence of SEQ ID NO: 332; or a heavy chain with an amino acid sequence of SEQ ID NO: 333 and optionally an Fc polypeptide with an amino acid sequence of SEQ ID NO: 356 and a light chain with an amino acid sequence of SEQ ID NO: 334; or a heavy chain with an amino acid sequence of SEQ ID NO: 335 and optionally an Fc polypeptide with an amino acid sequence of SEQ ID NO: 357 and a light chain with an amino acid sequence of SEQ ID NO: 336; or a heavy chain with an amino acid sequence of SEQ ID NO: 337 and optionally an Fc polypeptide with an amino acid sequence of SEQ ID NO: 358 and a light chain with an amino acid sequence of SEQ ID NO: 338; or a heavy chain with an amino acid sequence of SEQ ID NO: 339 and optionally an Fc polypeptide with an amino acid sequence of SEQ ID NO: 283 and a light chain with an amino acid sequence of SEQ ID NO: 340; or a heavy chain with an amino acid sequence of SEQ ID NO: 341 and optionally an Fc polypeptide with an amino acid sequence of SEQ ID NO: 359 and a light chain with an amino acid sequence of SEQ ID NO: 342.

331. The pharmaceutical composition of paragraph 330, wherein the transgene comprises a nucleotide sequence of SEQ ID NO: 129 encoding the heavy chain and a nucleotide sequence of SEQ ID NO: 130 encoding the light chain; or a nucleotide sequence of SEQ ID NO: 131 encoding the heavy chain and a nucleotide sequence of SEQ ID NO: 132 encoding the light chain; or a nucleotide sequence of SEQ ID NO: 343 encoding the heavy chain and a nucleotide sequence of SEQ ID NO: 344 encoding the light chain; or a nucleotide sequence of SEQ ID NO: 345 encoding the heavy chain and a nucleotide sequence of SEQ ID NO: 346 encoding the light chain; or a nucleotide sequence of SEQ ID NO: 347 encoding the heavy chain and a nucleotide sequence of SEQ ID NO: 348 encoding the light chain; or a nucleotide sequence of SEQ ID NO: 349 encoding the heavy chain and a nucleotide sequence of SEQ ID NO: 350 encoding the light chain; or a nucleotide sequence of SEQ ID NO: 351 encoding the heavy chain and a nucleotide sequence of SEQ ID NO: 352 encoding the light chain; or a nucleotide sequence of SEQ ID NO: 353 encoding the heavy chain and a nucleotide sequence of SEQ ID NO: 354 encoding the light chain.

332. The pharmaceutical composition of any of paragraphs 327 to 331, wherein the mAb or antigen-binding fragment thereof is a hyperglycosylated mutant or wherein the Fc polypeptide of the mAb is glycosylated or aglycosylated.

333. The pharmaceutical composition of any of paragraphs 327 to 331, wherein the transgene encodes a signal sequence at the N-terminus of the heavy chain and the light chain of said antigen-binding fragment that directs secretion and post translational modification in said human liver cells or human muscle cells.

334. The pharmaceutical composition of paragraph 333, wherein said signal sequence is MYRMQLLLLIALSLALVTNS (SEQ ID NO: 146) or a signal sequence from Table 3 or Table 4.

335. The pharmaceutical composition of any of paragraphs 327 to 334, wherein the AAV capsid is AAV8.

336. A method of reducing, inhibiting or ameliorating a detrimental immune response in a human subject in need thereof, comprising delivering to the circulation or tissue that is the target of the immune response of said human subject, a therapeutically effective amount of a substantially full-length or full-length mAb of an anti-interleukin-6 receptor (anti-IL6R) mAb, anti-interleukin-6 (IL6) mAb, or antigen-binding fragment thereof, expressed from a transgene and produced by human muscle or liver cells.

337. A method reducing, inhibiting or ameliorating a detrimental immune response in a human subject in need thereof, comprising:
administering to the muscle or liver of said human subject a therapeutically effective amount of a recombinant nucleotide expression vector comprising a transgene encoding a substantially full-length or full-length mAb of an anti-interleukin-6 receptor (anti-IL6R) mAb, anti-interleukin-6 (IL6) mAb, or an antigen-binding fragment thereof, operably linked to one or more regulatory sequences that control expression of the transgene in human muscle or liver cells, so that a depot is formed that releases a HuPTM form of the mAb or antigen-binding fragment thereof.

338. The method of paragraph 336 or 337 wherein the anti-IL6R is satralizumab, sarilumab, or tocilizumab, or the anti-IL6 mAb is siltuximab, clazakizumab, sirukumab, olokizumab, or gerilimzumab.

339. The method of any of paragraphs 336 to 338 wherein the antigen-binding fragment is a Fab, a F(ab)₂, or an scFv.

340. The method of any of paragraphs 336 to 339, wherein the full-length mAb or the antigen-binding fragment comprises a heavy chain with an amino acid sequence of SEQ ID NO: 59 and optionally an Fc polypeptide with an amino acid sequence of SEQ ID NO: 309 and a light chain with an amino acid sequence of SEQ ID NO: 60; or a heavy chain with an amino acid sequence of SEQ ID NO: 61 and optionally an Fc polypeptide with an amino acid sequence of SEQ ID NO: 310 and a light chain with an amino acid sequence of SEQ ID NO: 62; or a heavy chain with an amino acid sequence of SEQ ID NO: 341 and optionally an Fc polypeptide with an amino acid sequence of SEQ ID NO: 359 and a light chain with an amino acid sequence of SEQ ID NO: 342; or a heavy chain with an amino acid sequence of SEQ ID NO: 331 and optionally an Fc polypeptide with an amino acid sequence of SEQ ID NO: 355 and a light chain with an amino acid sequence of SEQ ID NO: 332; or a heavy chain with an amino acid sequence of SEQ ID NO: 333 and optionally an Fc polypeptide with an amino acid sequence of SEQ ID NO: 356 and a light chain with an amino acid sequence of SEQ ID NO: 334; or a heavy chain with an amino acid sequence of SEQ ID NO: 335 and optionally an Fc polypeptide with an amino acid sequence of SEQ ID NO: 357 and a light chain with an amino acid sequence of SEQ ID NO: 336; or a heavy chain with an amino acid sequence of SEQ ID NO: 337 and optionally an Fc polypeptide with an amino acid sequence of SEQ ID NO: 358 and a light chain with an amino acid sequence of SEQ ID NO: 338; or a heavy chain with an amino acid sequence of SEQ ID NO: 339 and optionally an Fc polypeptide with an IgG1 amino acid sequence of SEQ ID NO: 283 and a light chain with an amino acid sequence of SEQ ID NO: 340; or a heavy chain with an amino acid sequence of SEQ ID NO: 341 and optionally an Fc polypeptide with an amino acid sequence of SEQ ID NO: 359 and a light chain with an amino acid sequence of SEQ ID NO: 342.

341. The method of paragraph 340, wherein the transgene comprises a nucleotide sequence of SEQ ID NO: 129 encoding the heavy chain and a nucleotide sequence of SEQ ID NO: 130 encoding the light chain; or a nucleotide sequence of SEQ ID NO: 131 encoding the heavy chain and a nucleotide sequence of SEQ ID NO: 132 encoding the light chain; or a nucleotide sequence of SEQ ID NO: 343 encoding the heavy chain and a nucleotide sequence of SEQ ID NO: 344 encoding the light chain; or a nucleotide sequence of SEQ ID NO: 345 encoding the heavy chain and a nucleotide sequence of SEQ ID NO: 346 encoding the light chain; or a nucleotide sequence of SEQ ID NO: 347 encoding the heavy chain and a nucleotide sequence of SEQ ID NO: 348 encoding the light chain; or a nucleotide sequence of SEQ ID NO: 349 encoding the heavy chain and a nucleotide sequence of SEQ ID NO: 350 encoding the light chain; or a nucleotide sequence of SEQ ID NO: 351 encoding the heavy chain and a nucleotide sequence of SEQ ID NO: 352 encoding the light chain; or a nucleotide sequence of SEQ ID NO: 353 encoding the heavy chain and a nucleotide sequence of SEQ ID NO: 354 encoding the light chain.

342. The method of any of paragraphs 336 to 339, wherein the mAb or antigen-binding fragment thereof is a hyperglycosylated mutant or wherein the Fc polypeptide of the mAb is glycosylated or aglycosylated.

343. The method of any of paragraphs 336 to 342 wherein the mAb or antigen-binding fragment thereof contains an alpha 2,6-sialylated glycan.

344. The method of any of paragraphs 336 to 343 wherein the mAb or antigen-binding fragment thereof is glycosylated but does not contain detectable NeuGc or α-Gal.

345. The method of any of paragraphs 336 to 344 wherein the mAb or antigen-binding fragment thereof contains a tyrosine sulfation.

346. The method of any of paragraphs 336 to 345 wherein the recombinant expression vector is AAV8 or AAV9.

347. The method of any of paragraphs 336 to 346 in which production of said HuPTM form of said mAb or antigen-binding fragment thereof is confirmed by transducing human liver or muscle cells in culture with said recombinant nucleotide expression vector and expressing said mAb or antigen-binding fragment thereof.

### AAV Encoding Full Length mAb Composition Embodiments

348. A composition comprising an adeno-associated virus (AAV) vector having:
a. a viral AAV capsid, that is optionally at least 95% identical to the amino acid sequence of an AAV8 capsid (SEQ ID NO: 143), AAV9 capsid (SEQ ID NO: 144), AAVrh10 capsid (SEQ ID NO: 145), an AAVrh20 capsid, an AAVrh39 capsid or an AAVcy5 capsid; and
b. an artificial genome comprising an expression cassette flanked by AAV inverted terminal repeats (ITRs), wherein the expression cassette comprises a transgene encoding a substantially full-length or full-length mAb operably linked to one or more regulatory sequences that control expression of the transgene in human cells.
c. wherein the transgene encodes a signal sequence at the N-terminus of the heavy chain and the light chain of said mAb that directs secretion and post translational modification of said mAb.

349. The composition of paragraph 348, wherein said mAb comprises a heavy chain with a Fc polypeptide and a light chain having any one of the sequence combinations specified in paragraphs 4, 13, 22, 31, 40, 49, 58, 67, 76, 85, 95, 107, 119, 131, 143, 155, 167, 179, 191, 203, 222, 231, 240, 249, 258, 270, 282, 294, 307, and 317.

350. The composition of paragraph 348-349, wherein said mAb is a full length lanadelumab.

351. The composition of paragraph 350, wherein the transgene comprises a Furin/T2A linker between the nucleotide sequences coding for the heavy and light chains of said mAb.

352. The composition of paragraphs 350 to 351, wherein the regulatory sequence includes a regulatory sequence from Table 1.

353. The composition of paragraph 352, wherein the regulator sequence is a LMTP6 promoter, an ApoE.hAAT regulatory sequence, a CAG promoter, a CK8 regulatory sequence, or a TBG promoter.

354. The composition of paragraphs 350 to 353, wherein the transgene comprises a nucleotide sequence of SEQ ID NO. 141, 286, 287, or 435 to 444.

355. The composition of paragraphs 350 to 354, wherein the viral capsid is and AAV8 viral capsid.

356. A pharmaceutical composition for delivering lanadelumab to the bloodstream to treat hereditary angioedema in a human subject in need thereof, said composition comprising a recombinant AAV comprising a transgene encoding lanadelumab operably linked to one or more regulatory sequences that control expression of the transgene in muscle and/or liver cells, wherein said recombinant AAV is administered to said human subject at a dose sufficient to result in expression from the transgene and secretion of lanadelumab into the bloodstream of the human subject to produce lanadelumab plasma levels of at least 5 μg/ml to at least 35 μg/ml lanadelumab in said subject.

357. A method of treating hereditary angioedema in a human subject in need thereof, comprising administering to the subject a dose of a composition comprising a recombinant AAV comprising a transgene encoding lanadelumab operably linked to one or more regulatory sequences that control expression of the transgene in muscle and/or liver cells, in an amount sufficient to result in expression from the transgene and secretion of lanadelumab into the bloodstream of the human subject to produce lanadelumab plasma levels of at least 5 μg/ml to at least 35 μg/ml lanadelumab in said subject.

358. The method or composition of paragraphs 356 or 357 wherein the transgene comprises the nucleotide sequence of SEQ ID NO: 141, 286, 287, or 435 to 444

359. The method or composition of paragraphs 356 to 358, wherein the lanadelumab plasma levels are 20 μg/ml to 35 μg/ml.

360. The method or composition of paragraphs 356 to 359, wherein the lanadelumab plasma levels are maintained for at least three months.

361. The method or composition of paragraphs 356 to 360 wherein the lanadelumab antibody secreted into the plasma exhibits greater a greater than at least 40%, 45%, 50%, 55%, 60%, 65% or 70 reduction in pKal activity as measured by a kinetic enzymatic functional assay.

362. The method or composition of paragraph 361 wherein the activity of the lanadelumab antibody is measured at 2 weeks, 3 weeks, 4 weeks, 5 weeks, 6 weeks, 7 weeks, 8 weeks, 9 weeks, 10 weeks, 11 weeks or 12 weeks after said administration.

363. A method of determining human anti-pKal antibody activity in a sample, said method comprising
a. Incubating the sample with activated human pKal;
b. Subsequently incubating the sample having been incubated with the activated human pKal with the synthetic substrate Pro-Phe-Arg-AMC
c. Measure the release of AMC over three hours as compared to a control sample.

### Additional Embodiments

364. A pharmaceutical composition for delivery of an antibody or antigen binding fragment thereof to the bloodstream of a human subject in need thereof, comprising an AAV vector comprising:
(a) An AAV viral capsid that infects liver and/or muscle cells; and
(b) an artificial genome comprising an expression cassette flanked by AAV ITRs wherein the expression cassette comprises a transgene encoding a full-length antibody or an antigen-binding fragment thereof, operably linked to a chimeric promoter that directs expression in muscle and liver cells;
wherein said AAV vector is formulated for intramuscular administration.

365. The pharmaceutical composition of paragraph 364 wherein the chimeric promoter is LMTP6 (SEQ ID NO: 320), LMTP13 (SEQ ID NO: 321), LMTP14 (SEQ ID NO: 322), LMTP15 (SEQ ID NO: 323), LMTP18 (SEQ ID NO: 324), LMTP19 (SEQ ID NO: 325), or LMTP20 (SEQ ID NO: 326).

366. The pharmaceutical composition of paragraph 365 wherein the chimeric promoter is LMPT6 (SEQ ID NO: 320).

367. The pharmaceutical composition of any one of paragraphs 364 to 366, wherein the AAV viral capsid is at least 95% identical to the amino acid sequence of an AAV8 capsid (SEQ ID NO: 143), an AAV9 capsid (SEQ ID NO: 144), an AAVrh10 capsid (SEQ ID NO: 145).

368. The pharmaceutical composition of any of paragraphs 364 to 3673, wherein the antibody is sevacizumab, LKA-651, ravulizumab, adalimumab, infliximab, golimumab, elezanumab, NI-301, PRX-004, pamrevlumab, siltuximab, clazakizumab, sirukumab, olokizumab, gerilimzumab, satralizumab, sarilumab, tocilizumab, inebilizumab, etrolizumab, romosozumab, lanadelumab, benralizumab, reslizumab, tralokinumab, nemolizumab, omalizumab, or tezepelumab.

369. The pharmaceutical composition of any of paragraphs 364 to 366, wherein the transgene comprises nucleotide sequence SEQ ID NO; 443.

### 4. BRIEF DESCRIPTION OF THE DRAWINGS

FIG. 1. A schematic of an rAAV vector genome construct containing an expression cassette encoding the heavy and light chains of the Fab region of a therapeutic mAb controlled by expression elements, flanked by the AAV ITRs.

FIGS. 2A-C. The amino acid sequence of a transgene construct for the Fab region of therapeutic antibodies to amyloid 13 peptides: solanezumab Fab (FIG. 2A), GSK933776 (FIG. 2B) and lecanemab (FIG. 2C). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The heavy chain hinge regions are highlighted in grey.

FIG. 3. The amino acid sequence of a transgene construct for the Fab region of therapeutic antibodies to sortilin: AL-001 (FIG. 3). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The heavy chain hinge regions are highlighted in grey.

FIGS. 4A-4C. The amino acid sequence of a transgene construct for the Fab region of therapeutic antibodies to tau: ABBV-8E12 (FIG. 4A), UCB-0107 (FIG. 4B), and NI-105 (FIG. 4C). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The heavy chain hinge regions are highlighted in grey.

FIG. 5. The amino acid sequence of a transgene construct for the Fab region of therapeutic antibodies to SEMA4D: VX15/2503 (FIG. 5). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge regions are highlighted in grey.

FIGS. 6A-C. The amino acid sequence of a transgene construct for the Fab region of therapeutic antibodies to alpha-synuclein: prasinezumab (FIG. 6A), NI-202 (FIG. 6B), and MEDI-1341 (FIG. 6C). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIGS. 7A and B. The amino acid sequence of a transgene construct for the Fab region of therapeutic antibodies to superoxide dismutase 1 (SOD1): NI-205.10D12 (FIG. 7A); and NI-205.12G7 (FIG. 7B). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge regions are highlighted in grey.

FIGS. 8A-C. The amino acid sequence of a transgene construct for the Fab region of therapeutic antibodies directed at CGRPR: eptinezumab (FIG. 8A), fremanezumab (FIG. 8B), and galcanezumab (FIG. 8C). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge regions are highlighted in grey.

FIGS. 9A-C. The amino acid sequence of a transgene construct for the Fab region of therapeutic antibody directed at biological factors: anti-VEGF, sevacizumab (FIG. 9A); anti-EpoR, LKA-651.NVS2 (FIG. 9B), and LKA-651.NVS3 (FIG. 9C). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge regions are highlighted in grey.

FIGS. 10A-D. The amino acid sequence of a transgene construct for the Fab region of therapeutic antibody directed at biological factors: anti-ALK1, ascrinvacumab (FIG. 10A); anti-C5, tesidolumab (FIG. 10B) and ravulizumab (FIG. 10D), and anti-endoglin, carotuximab (FIG. 10C). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The heavy chain hinge regions are highlighted in grey.

FIG. 11. The amino acid sequence of a transgene construct for the Fab region of ANX-007, a therapeutic antibody to CC1Q. Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. The hinge region is highlighted in grey.

FIGS. 12A-C. The amino acid sequence of a transgene construct for the Fab region of therapeutic antibody directed at TNF-alpha: adalimumab (FIG. 12A), infliximab (FIG. 12B), and golimumab (FIG. 12C). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The heavy chain hinge regions are highlighted in grey.

FIG. 13. The amino acid sequence of a transgene construct for the Fab region of elezanumab, a therapeutic antibody to RGMa. Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIGS. 14A and B. The amino acid sequence of a transgene construct for the Fab region of therapeutic antibody directed at transthyretin (TTR): NI-301 (FIG. 14A) and PRX-004 (FIG. 14B). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIG. 15. The amino acid sequence of a transgene construct for the Fab region of pamrevlumab, a therapeutic antibody to CTGF. Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIGS. 16A-I. The amino acid sequence of a transgene construct for the Fab region of therapeutic antibody directed at biological factors: anti-IL6R, satralizumab (FIG. 16A), sarilumab (FIG. 16B), tocilizumab (FIG. 16H); anti-IL6, siltuximab (FIG. 16C), clazakizumab (FIG. 16D), sirukumab (FIG. 16E), olokizumab (FIG. 16F), gerilimzumab (FIG. 16G), and anti-CD19, inebilizumab (FIG. 16I). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIG. 17. The amino acid sequence of a transgene construct for the Fab region of etrolizumab, a therapeutic antibody to ITGB7. Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIG. 18. The amino acid sequence of a transgene construct for the Fab region of romosozumab, a therapeutic antibody to sclerostin. Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIG. 19. The amino acid sequence of a transgene construct for the Fab region of lanadelumab, a therapeutic antibody to plasma kallikrein (pKal). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIGS. 20A and B. Amino acid sequence alignment of the amino acid sequences of the heavy (FIG. 20A) (residues 1-220 of SEQ ID NO: 1, residues 1-223 of SEQ ID NO: 3, residues 1-237 of SEQ ID NO: 5, residues 1-220 of SEQ ID NO: 7, residues 1-223 of SEQ ID NO: 9, residues 1-232 of SEQ ID NO: 11, residues 1-228 of SEQ ID NO: 13, residues 1-224 of SEQ ID NO: 15, residues 1-232 of SEQ ID NO: 17, residues 1-230 of SEQ ID NO: 19, residues 1-234 of SEQ ID NO: 21, residues 1-231 of SEQ ID NO: 23, residues 1-219 of SEQ ID NO: 25, residues 1-227 of SEQ ID NO: 27, residues 1-224 of SEQ ID NO: 29, residues 1-230 of SEQ ID NO: 31, residues 1-225 of SEQ ID NO: 33, residues 1-235 of SEQ ID NO: 35, residues 1-223 of SEQ ID NO: 37, residues 1-224 of SEQ ID NO: 39, residues 1-226 of SEQ ID NO: 41, residues 1-229 of SEQ ID NO: 43, residues 1-229 of SEQ ID NO: 45, residues 1-228 of SEQ ID NO: 47, residues 1-237 of SEQ ID NO: 49, residues 1-228 of SEQ ID NO: 51, residues 1-228 of SEQ ID NO: 53, residues 1-225 of SEQ ID NO: 55, residues 1-224 of SEQ ID NO: 57, residues 1-224 of SEQ ID NO: 59, residues 1-224 of SEQ ID NO: 61, residues 1-229 of SEQ ID NO: 63, residues 1-225 of SEQ ID NO: 65, residues 1-228 of SEQ ID NO: 67, residues 1-230 of SEQ ID NO: 69, residues 1-227 of SEQ ID NO: 331, residues 1-228 of SEQ ID NO: 333, residues 1-227 of SEQ ID NO: 335, residues 1-224 of SEQ ID NO: 337, residues 1-230 of SEQ ID NO: 339, residues 1-228 of SEQ ID NO: 341, residues 1-232 of SEQ ID NO: 360, residues 1-227 of SEQ ID NO: 362, residues 1-229 of SEQ ID NO: 364, residues 1-221 of SEQ ID NO: 366, residues 1-226 of SEQ ID NO: 368, residues 1-226 of SEQ ID NO: 370, residues 1-225 of SEQ ID NO: 372, and residues 1-227 of SEQ ID NO: 374, respectively, in order of appearance) and light (FIG. 20B) (SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 332, 334, 336, 338, 340, 342, 361, 363, 365, 367, 369, 371, 373, and 375, respectively, in order of appearance) chain Fab portions of the therapeutic antibodies disclosed herein. Positions that may be substituted to produce hyperglycosylated variants of the Fab regions are highlighted. Four substitutions (one in the heavy chain and three in the light chain) that should result in hyperglycosylation of the Fab region by human cells are annotated above the amino acid residue positions. (For engineering mAbs or antigen-binding fragments to contain additional glycosylation sites on the Fab domain, see e.g., Courtois et al., 2016, mAbs 8: 99-112 for a description of derivatives of antibodies that are hyperglycosylated on the Fab domain of the full-length antibody).

FIG. 21. Clustal Multiple Sequence Alignment of AAV capsids 1-9. Amino acid substitutions (shown in bold in the bottom rows) can be made to AAV9 and AAV8 capsids by “recruiting” amino acid residues from the corresponding position of other aligned AAV capsids. Sequence shown in gray=hypervariable regions. The amino acid sequences of the AAV capsids are assigned SEQ ID NOs as follows: AAV1 is SEQ ID NO: 274; AAV2 is SEQ ID NO: 275; AAV3-3 is SEQ ID NO: 276; AAV4-4 is SEQ ID NO: 277; AAV5 is SEQ ID NO: 278; AAV6 is SEQ ID NO: 279; AAV7 is SEQ ID NO: 280; AAV8 is SEQ ID NO: 143; AAV9 is SEQ ID NO: 144; AAVrh10 is SEQ ID NO: 145; hu31 is SEQ ID NO: 281; and hu32 is SEQ ID NO: 282.

FIG. 22. Glycans that can be attached to HuGlyFab regions of full length mAbs or the antigen-binding domains. (Adapted from Bondt et al., 2014, Mol & Cell Proteomics 13.1: 3029-3039).

FIG. 23. Clustal Multiple Sequence Alignment of constant heavy chain regions (CH2 and CH3) of IgG1 (SEQ ID NO: 283), IgG2 (SEQ ID NO: 284), and IgG4 (SEQ ID NO: 285). The hinge region, from residue 219 to residue 230 of the heavy chain, is shown in italics. The numbering of the amino acids is in EU-format.

FIGS. 24A-D. A. Schematic showing the genome configuration of AAV8 and AAV9. The expression cassette utilizes the CAG promoter (SEQ ID NO: 411) to drive expression of a human antibody that binds to and inhibits for example, plasma kallikrein (pKal) or TNFα. A mutant IL2 leader (mIL2, SEQ ID NO: 146) targets the heavy and light chains for secretion and the furin-F2A sequence (SEQ ID NO: 231) drives the cleavage of the polyprotein into heavy and light chain components. B. Transfection titration comparing CAG.L01 (SEQ ID NO: 435; containing lanadelumab sequence L01 (SEQ ID NO: 141)) and CAG.L02 (SEQ ID NO: 437; containing lanadelumab sequence L02 (SEQ ID NO: 286)) proviral plasmid constructions. Top panels demonstrate reporter transgene (eGFP) expression following transfection of different plasmid quantities (4 μg-nontransfected). Bottom left panel depicts lanadelumab expression in the cell lysate while the bottom right panel detects plasmid expressed lanadelumab secreted into the cell supernatant. C. Transfection titration comparing CAG.L02 and CAG.L03 proviral plasmid constructions. Panels depict different exposure lengths (30 seconds or 60 seconds) of expressed lanadelumab from CAG.L02 or CAG.L03 constructs secreted into the cell supernatant. D. Transfection titration comparing Lanadelumab Fab proviral plasmid constructions. Figure depicts levels of Lanadelumab Fab following transfection of different plasmid quantities. L01 construct (CAG.L01: SEQ ID NO: 435) is driven by the CB promoter, while L02 (CAG. L02: SEQ ID NO: 437) is driven by the CAG promoter (SEQ ID NO: 411).

FIG. 25. The indicated AAV9 and AAV8 vectors (n=5 per group) were administered to NGS mice via either intravenous (IV) or intramuscular (IM) routes. IV administrations were into the tail vein and IM administrations were bilateral into the gastrocnemius muscles. Mice treated with vehicle were included as controls. Seven weeks post administration mice were sacrificed, and serum human antibody levels were determined by ELISA.

FIG. 26. A time course of antibody expression (lanadelumab serum levels) in NGS mice post-AAV9 administration (n=5 per group) is shown. AAV9 vectors (2e11 gc) were injected either IV or IM and serum antibody levels were determined by ELISA at day 7 (D7), day 21 (D21), day 35 (D35), and day 49 (D49).

FIG. 27 depicts the expression of the monoclonal antibody lanadelumab (Mab 1) in C2C12 muscle cells upon transduction of the cells with different cis plasmids expressing lanadelumab under the control of different regulatory elements: CAG (SEQ ID NO: 411), LMTP6 (SEQ ID NO: 320), and ApoE.hAAT (SEQ ID NO: 412). For detection of antibody protein, following transduction, the cells were treated with FITC conjugated anti-Fc (IgG) antibody. DAPI staining is shown to confirm confluency and viability of the cells under all conditions tested.

FIGS. 28A and B. A Serum expression levels (μg/ml) of lanadelumab upon intravenous injection of C/57BL6 mice with 2.5×10¹² vg/kg of AAV8 vectors encoding a lanadelumab regulated by different liver-specific, liver-tandem and liver-muscle regulatory elements (see Table 1). CAG (SEQ ID NO: 411) and TBG (SEQ ID NO: 423) promoters were used as controls. Data from the blood draw at 1, 3, 5 and 7 weeks post injection are shown. LSPX1, liver-specific promoter 1 (SEQ ID NO: 315); LSXP2, liver-specific promoter 2 (SEQ ID NO: 316); LTP1, liver-specific tandem promoter 1 (SEQ ID NO: 317); LMTP6, liver and muscle dual-specific tandem promoter 6 (SEQ ID NO: 320). Protein expression levels were quantified by ELISA from biweekly serum collections. N=5 mice per vector. Numbers on x-axis represent the weeks post vector administration. Data represent mean+SEM. 8B. Quantification of viral genomes in liver. C57Bl/6 mice were administrated intravenously with AAV8 vectors driven by different liver-specific promoters at equivalent doses (2.5×10¹² vg/kg). N=5 mice per group. Vector DNA was analyzed by ddPCR in mouse liver samples collected at 49 days post vector administration. Data represent mean+SEM.

FIGS. 29A-F. The amino acid sequence of a transgene construct for the Fab region of therapeutic antibody directed at biological factors: anti-IL5, benralizumab (A); anti-IL5R, reslizumab (B); anti-IL13, tralokinumab (C); anti-IL31R, nemolizumab (D), anti-IgE, omalizumab (E), and anti-TSLP, tezepelumab (F). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIGS. 30A and 30B. A. Route of administration and dose selection in Wistar rats. AAV8 vectors encoding vectorized lanadelumab driven by CAG promoters were injected intramuscularly at 1×10¹³ vg/kg (body weight) or intravenously at 1×10¹³ vg/kg and 1×10¹⁴ vg/kg into SD rats. Protein expression was quantified by ELISA from serum collected every three to seven days. N=3 rats per vector. Data represent mean+SEM. * indicates p<0.05, ** indicates p<0.01 with Welch's t test. B. AAV8 vectors encoding vectorized lanadelumab driven by CAG (SEQ ID NO: 411) or ApoE.hAAT (SEQ ID NO: 412) promoters were injected intravenously at 5×10¹³ vg/kg into Wistar and SD rats. Protein expression was quantified by ELISA from weekly serum collection. N=3 rats per vector. Data represent mean+SEM. P value: *, p<0.05; **, p<0.01. Serum antibody concentrations (mean and SEM) in animals of each group at each time point are presented in the table.

FIGS. 31A-31D. A. Serum anti-kallikrein (pKal) (lanadelumab) antibody concentration following AAV8 delivery. Animals received bilateral injections of 5×10¹⁰ vg/kg into the GA muscle. Serum was collected biweekly and vectorized antibody concentration was quantified with ELISA. B. Vector genome quantification from relevant tissues with digital droplet PCR (ddPCR). C. Comparison of vector gene expression from liver. Data represent relative fold gene expression as quantified by the ΔΔCT method. D. Comparison of AAV transgene expression from tissues using digital droplet PCR (ddPCR). Anti-pKal antibody mRNA copies were normalized to GAPDH mRNA copies across tissues. Data are represented as mean±SEM. Statistical significance was determined using a one-way ANOVA followed by Tukey's HSD post-test. *P<0.05, **P<0.01.

FIG. 32: Antibody concentrations in the serum of wild type mice treated with AAV8.Lanadelumab vectors produced with different BV/Sf9 production systems compared to an HEK system. C57BL/6 mice were intravenously injected with vectors at a dose of 2.5×10¹² vg/kg.

FIGS. 33A-33F. A and B show the pKal titration curve and signal-to-noise ratios for indicated pKal concentrations. C. Two pKal concentrations (6.25 nM and 12.5 nM) were used to measure the suppressive range of lanadelumab (compared to non-specific human IgG control antibody) in an antibody-dose response. C57BL/6 mice (n=5) were administered 5×10¹⁰ vector genomes (vg) (2.5×10¹² vg/kg) of ApoE.hAAT.L02.AAV8 per mouse intravenously. Shown are the compiled enzyme activities and percent reductions in pKal activity for both mouse groups D and E. The slopes of enzymatic progressive activity curves and an AMC standard were used to calculate specific pKal enzymatic activity, where significantly less activity was recorded at day 49 compared to day −7. F. The percent reduction in enzymatic activity was calculated as day 49 activity divided by that of day −7. Vectorized anti-pKal antibody-containing IgG significantly reduced pKal activity. All results are a compilation of 2-5 mice per group. To determine significance of differences, Student's t-tests were used (paired, two-tailed), where *p<0.05, **p<0.01, ***p<0.001.

FIGS. 34A-34L. Quantification of mouse paw volumes and paw swellings in carrageenan-induced paw edema mice treated with test articles. Bar charts show the paw volumes (A, C, E, G, I, and K) measured at 2 (A), 4 (C), 6 (E), 8 (G), 24 (I) and 48 (K) hours after carrageenan injection in C57BL/6 mice. Paw swelling difference (B, D, F, H, J, and L) was evaluated by calculating the difference of paw volumes measured at each time point and the baseline. N=10 mice per group. Data analysis was done with One-way ANOVA with Dunnett's post-hoc test for multiple comparisons. Data represent mean+S.DEM. P values: *, p<0.05; **, p<0.01; ***, p<0.001; ****, p<0.0001.

FIGS. 35A and 35B: Time course of mouse paw volumes measured in carrageenan-induced paw edema mice treated with test articles. Mouse paw volumes were measured before (baseline) and at different time points after 0.7% (A) or 1% (B) carrageenan injection. N=10 mice per group. Data represent mean±SEM.

FIGS. 36A and 36B: Characterization of vectorized adalimumab IgG and Fab cis plasmid expression. (A) Western blot depicting expression of adalimumab IgG and Fab derived from cell supernatant of 293T cells transfected with the respective cis plasmids. (B) Human TNFα binding ELISA derived from cells transfected with the cis plasmid. pAAV.CAG.Lanadelumab.IgG was used as a non-specific antibody (mAb) control. Data represented as mean±SEM.

FIGS. 37A-37C. Characterization of AAV8 expressed adalimumab IgG expression and activity. (A) Quantification of AAV8 expressed adalimumab at two multiplicity of infection (MOI) following transduction of 293T.AAVR cells. (B) Western blot depicting expression of adalimumab IgG heavy and light chain components at two different MOIs. (C) Human TNFα binding ELISA of adalimumab IgG derived from cell culture supernatant. Data represented as mean±SEM.

FIG. 38. Comparison of self-complementary AAV cis plasmids encoding vectorized adalimumab Fab. Negative control includes cell supernatant from non-transfected cells. Data represented as mean±SEM.

FIG. 39. Binding of TNFα across model species (mouse, rat, and human) by vectorized adalimumab IgG and Fab. Negative control included supernatant derived from non-transfected cells. Vectorized lanadelumab (pAAV.CAG.lanadelumab.IgG) functioned as a non-specific antibody control. Data represented as mean±SEM.

### 5. DETAILED DESCRIPTION OF THE INVENTION

Compositions and methods are described for the delivery of a fully human post-translationally modified (HuPTM) therapeutic monoclonal antibody (mAb) or a HuPTM antigen-binding fragment of a therapeutic mAb (for example, a fully human-glycosylated Fab (HuGlyFab) of a therapeutic mAb) to a patient (human subject) diagnosed with a disease or condition indicated for treatment with the therapeutic mAb. Delivery may be advantageously accomplished via gene therapy—e.g., by administering a viral vector or other DNA expression construct encoding a therapeutic mAb or its antigen-binding fragment (or a hyperglycosylated derivative of either) to a patient (human subject) diagnosed with a condition indicated for treatment with the therapeutic mAb—to create a permanent depot in a tissue or organ of the patient that continuously supplies the HuPTM mAb or antigen-binding fragment of the therapeutic mAb, e.g., a human-glycosylated transgene product, to a target tissue where the mAb or antigen-binding fragment there of exerts its therapeutic effect.

The HuPTM mAb or HuPTM antigen-binding fragment encoded by the transgene can include, but is not limited to, a full-length or an antigen-binding fragment of a therapeutic antibody that binds to:
Nervous system targets, including amyloid beta (Aβ or Abeta) peptides, sortilin, tau protein, SEMA4D, alpha-synuclein, and CGRP receptor,
Ocular Targets including VEGF, EpoR, ALK1, endoglin, complement component 5, and complement component 1Q,
Repulsive guidance molecule-A
Transthyretin
Connective Tissue Growth Factor
Neuromyelitis optica (NMO)/non-infectious uveitis targets and immune response targets, including interleukin 6 receptor, interleukin 6, and CD19
Integrin beta 7
Sclerostin
TNF-alpha, and
Plasma Protein Targets, such as human complement proteins, including plasma kallikrein,
Autoimmune, respiratory, and allergic disease targets, such as interleukin and interleukin receptors, including IL5, IL5R, IL13, and IL31RA, immunoglobulin E, and thymic stromal lymphopoietin
   or such mAbs or antigen-binding fragments engineered to contain additional glycosylation sites on the Fab domain (e.g., see Courtois et al., 2016, mAbs 8: 99-112 which is incorporated by reference herein in its entirety for its description of derivatives of antibodies that are hyperglycosylated on the Fab domain of the full-length antibody). The amino acid sequences of the heavy and light chains of antigen binding fragments of the foregoing are provided in Table 5, infra, and nucleotide sequences, including codon optimized versions, encoding the heavy and light chains of these antigen binding fragments are provided in Table 6

The recombinant vector used for delivering the transgene includes non-replicating recombinant adeno-associated virus vectors (“rAAV”) rAAVs are particularly attractive vectors for a number of reasons—they can transduce non-replicating cells, and therefore, can be used to deliver the transgene to tissues where cell division occurs at low levels, such as the CNS; they can be modified to preferentially target a specific organ of choice; and there are hundreds of capsid serotypes to choose from to obtain the desired tissue specificity, and/or to avoid neutralization by pre-existing patient antibodies to some AAVs. Such rAAVs include but are not limited to AAV based vectors comprising capsid components from one or more of AAV1, AAV2, AAV2.m78, AAV3, AAV4, AAV5, AAV6, AAV7, AAV8, AAV9, AAV10, AAV11, AAVrh10 or AAVrh20. In certain embodiments, AAV based vectors provided herein comprise capsids from one or more of AAV8, AAV9, AAV10, AAV11, AAVrh10 or AAVrh20 serotypes.

However, other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements.

Gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. In certain embodiments, the coding sequences encode for a Fab or F(ab′)₂ or an scFv.

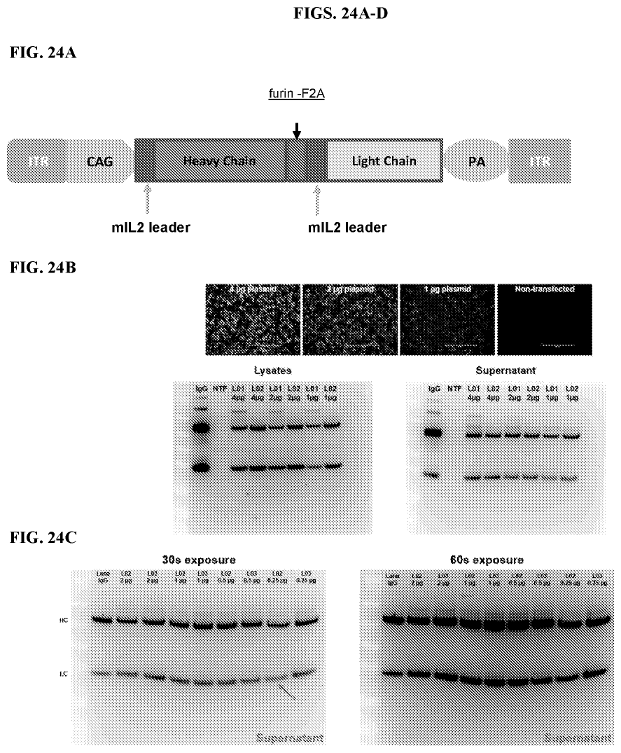

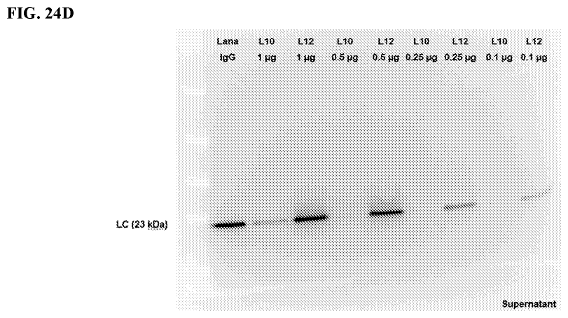

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161) and may also be optimized to reduce CpG dimers. Nucleotide sequences of the heavy and light chain variable domains of the therapeutic antibodies, which may be codon optimized, are disclosed in Table 6. Each heavy and light chain requires a leader to ensure proper post-translation processing and secretion (unless expressed as an scFv, in which only the N-terminal chain requires a leader sequence). Useful leader sequences for the expression of the heavy and light chains of the therapeutic antibodies in human cells are disclosed herein. An exemplary recombinant expression construct is shown in FIG. 1 and in FIG. 24A.

The production of HuPTM mAb or HuPTM Fab (including an HuPTM scFv) should result in a “biobetter” molecule for the treatment of disease accomplished via gene therapy—e.g., by administering a viral vector or other DNA expression construct encoding a full-length HuPTM mAb or HuPTM Fab or other antigen binding fragment, such as an scFv, of a therapeutic mAb to a patient (human subject) diagnosed with a disease indication for that mAb, to create a permanent depot in the subject that continuously supplies the human-glycosylated, sulfated transgene product produced by the subject's transduced cells. The cDNA construct for the HuPTMmAb or HuPTM Fab or HuPTM scFv should include a signal peptide that ensures proper co- and post-translational processing (glycosylation and protein sulfation) by the transduced human cells.

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil.

As an alternative, or an additional treatment to gene therapy, the full-length HuPTM mAb or HuPTM Fab or other antigen binding fragment thereof can be produced in human cell lines by recombinant DNA technology, and the glycoprotein can be administered to patients. Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. 36(6):1110-1122, which is incorporated by reference in its entirety for a review of the human cell lines that could be used for the recombinant production of the HuPTM mAb, HuPTM Fab or HuPTM scFv product, e.g., HuPTM Fab glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in human cells.

It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment of the invention is to slow or arrest the progression of disease.

Combination therapies involving delivery of the full-length HuPTM mAb or HuPTM Fab or antigen binding fragment thereof to the patient accompanied by administration of other available treatments are encompassed by the methods of the invention. The additional treatments may be administered before, concurrently or subsequent to the gene therapy treatment. Such additional treatments can include but are not limited to co-therapy with the therapeutic mAb.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

### 5.1 Constructs

Viral vectors or other DNA expression constructs encoding an HuPTM mAb or antigen-binding fragment thereof, particularly a HuGlyFab, or a hyperglycosylated derivative of a HuPTM mAb antigen-binding fragment are provided herein. The viral vectors and other DNA expression constructs provided herein include any suitable method for delivery of a transgene to a target cell. The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to retinal pigment epithelial cells, CNS cells, muscle cells, or liver cells.

In some aspects, the disclosure provides for a nucleic acid for use, wherein the nucleic acid comprises a nucleotide sequence that encodes a HuPTM mAb or HuGlyFab or other antigen-binding fragment thereof, as a transgene described herein, operatively linked to a promoter selected for expression in tissue targeted for expression of the transgene, for example, but not limited to the CB7/CAG promoter (SEQ ID NO: 411, FIG. 24A) and associated upstream regulatory sequences (see FIG. 1), cytomegalovirus (CMV) promoter, Rous sarcoma virus (RSV) promoter, GFAP promoter (glial fibrillary acidic protein), MBP promoter (myelin basic protein), MMT promoter, EF-1 alpha promoter (SEQ ID NO: 415), mU1a (SEQ ID NO: 414), UB6 promoter, chicken beta-actin (CBA) promoter, RPE65 promoter and opsin promoter, liver-specific promoters, such as TBG (Thyroxine-binding Globulin) promoter (SEQ ID NO: 423), APOA2 promoter, SERPINA1 (hAAT) promoter, ApoE.hAAT (SEQ ID NO: 412) or mIR122 promoter, or muscle-specific promoters, such as a human desmin promoter, CK8 promoter (SEQ ID NO: 413) or Pitx3 promoter, inducible promoters, such as a hypoxia-inducible promoter or a rapamycin-inducible promoter.

In some aspects herein, transgene expression is controlled by engineered nucleic acid regulatory elements that have more than one regulatory element (promoter or enhancer), including regulatory elements that are arranged in tandem (two or three copies) that promote liver-specific expression, or both liver-specific expression and muscle-specific expression, or both liver-specific and expression and bone-specific expression. These regulatory elements include for the liver-specific expression, LSPX1 (SEQ ID NO: 315), LSPX2 (SEQ ID NO: 316), LTP1 (SEQ ID NO: 317), LTP2 (SEQ ID NO: 318), or LTP3 (SEQ ID NO: 319), liver and muscle expression, LMTP6 (SEQ ID NO: 320), LMTP13 (SEQ ID NO: 321), LMTP14 (SEQ ID NO: 322), LMTP15 (SEQ ID NO: 323), LMTP18 (SEQ ID NO: 324), LMTP19 (SEQ ID NO: 325), or LMTP20 (SEQ ID NO: 326), or liver and bone expression, LBTP1 (SEQ ID NO: 327) or LBTP2 (SEQ ID NO: 328), the sequences of which are provided in Table 1.

In certain embodiments, provided herein are recombinant vectors that comprise one or more nucleic acids (e.g., polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., the nucleotide sequences encoding the heavy and light chains of the HuPTMmAb or HuGlyFab or other antigen-binding fragment), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161). Nucleotide sequences for expression in human cells are provided herein for the heavy and light chains of the HuGlyFabs in Table 6.

In a specific embodiment, the constructs described herein comprise the following components: (1) AAV2 inverted terminal repeats that flank the expression cassette; (2) one or more control elements, b) a chicken β-actin intron and c) a rabbit β-globin poly A signal; and (3) nucleic acid sequences coding for the heavy and light chains of a mAb or Fab, separated by a self-cleaving furin (F)/(F/T)2A linker (SEQ ID NOS: 231 or 429), ensuring expression of equal amounts of the heavy and the light chain polypeptides. An exemplary construct is shown in FIG. 1.

In a specific embodiment, the constructs described herein comprise the following components: (1) AAV2 inverted terminal repeats that flank the expression cassette; (2) one or more control elements, b) a chicken β-actin intron and c) a rabbit β-globin poly A signal; and (3) nucleic acid sequences coding for a full-length antibody comprising the heavy and light chain sequences using sequences that encode the Fab portion of the heavy chain, including the hinge region sequence, plus the Fc polypeptide of the heavy chain for the appropriate isotype and the light chain, wherein heavy and light chain nucleotide sequences are separated by a self-cleaving furin (F)/(F/T)2A linker (SEQ ID NOS: 231 or 429), ensuring expression of equal amounts of the heavy and the light chain polypeptides. An exemplary construct is shown in FIG. 24A.

### 5.1.1 mRNA Vectors

In certain embodiments, as an alternative to DNA vectors, the vectors provided herein are modified mRNA encoding for the gene of interest (e.g., the transgene, for example, HuPTMmAb or HuGlyFab or other antigen binding fragment thereof). The synthesis of modified and unmodified mRNA for delivery of a transgene to retinal pigment epithelial cells is taught, for example, in Hansson et al., J. Biol. Chem., 2015, 290(9):5661-5672, which is incorporated by reference herein in its entirety. In certain embodiments, provided herein is a modified mRNA encoding for a HuPTMmAb, HuPTM Fab, or HuPTM scFv.

### 5.1.2 Viral Vectors

Viral vectors include adenovirus, adeno-associated virus (AAV, e.g., AAV8, AAV9, AAVrh10, AAV2.7m8), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV) and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

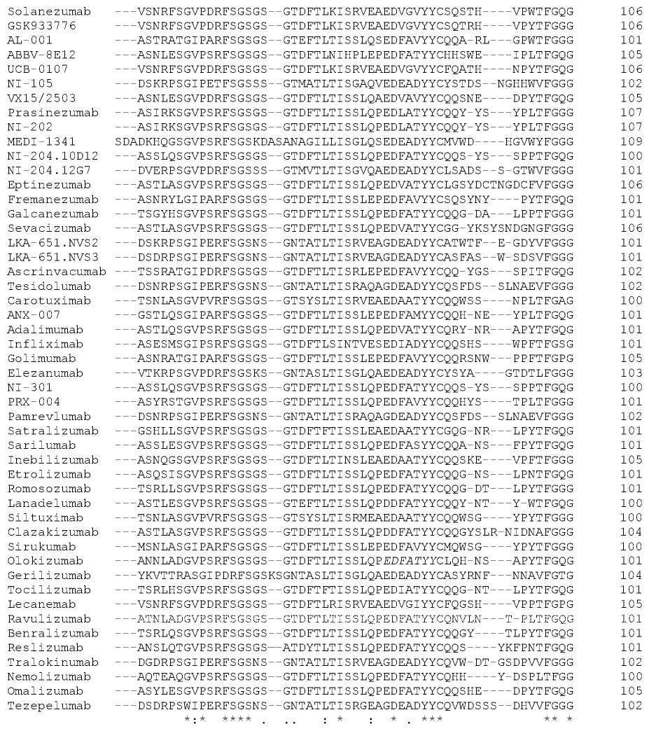

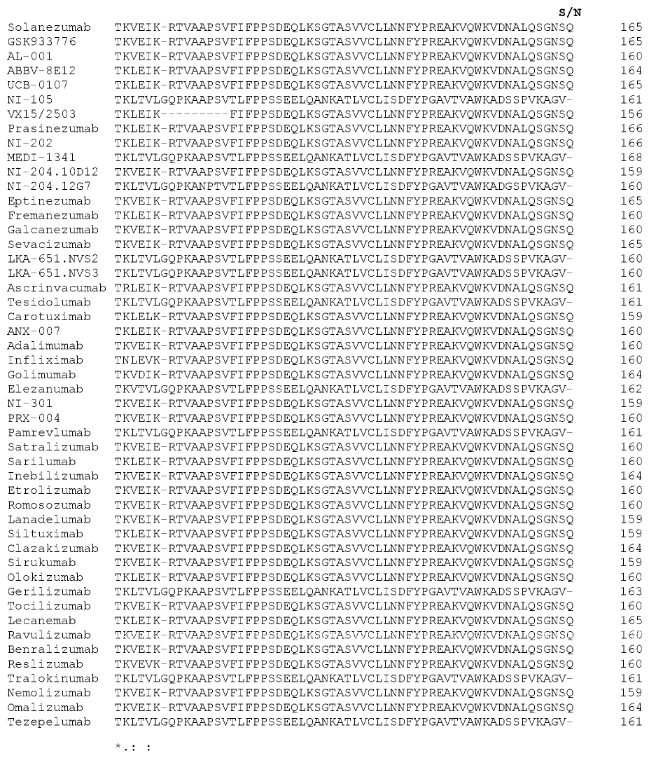

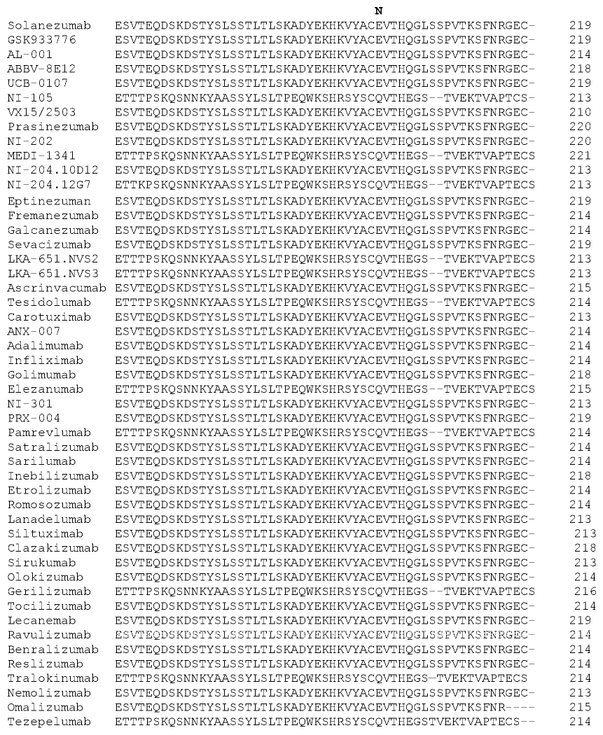

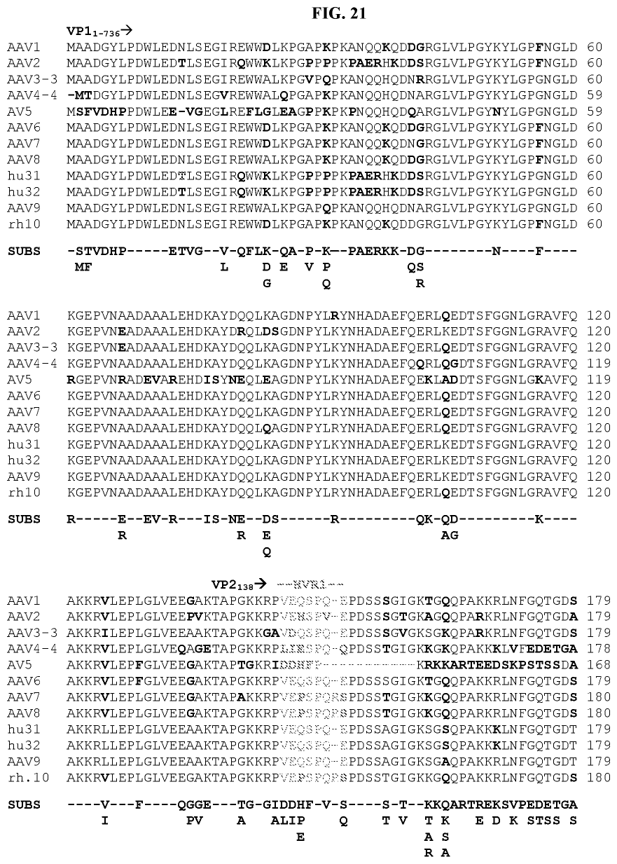

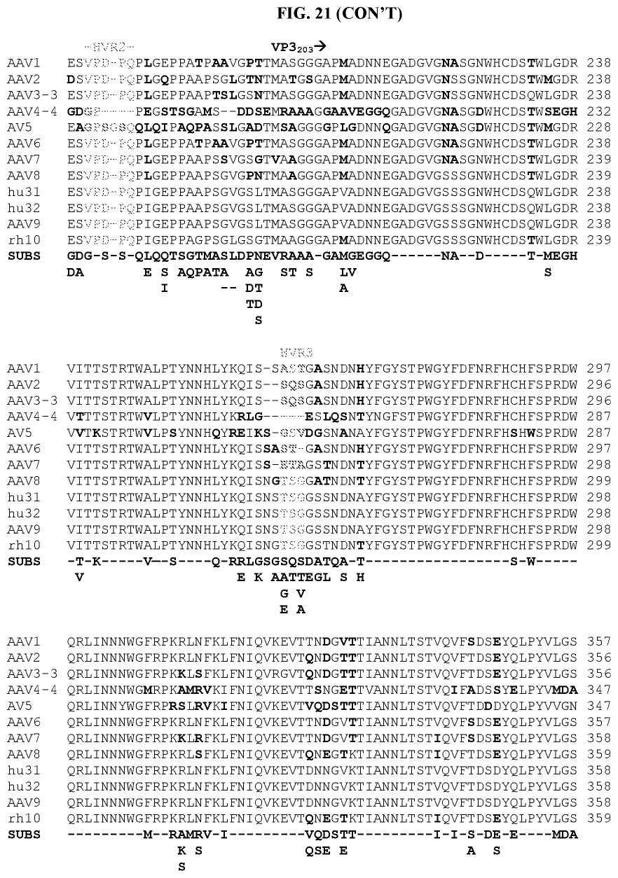

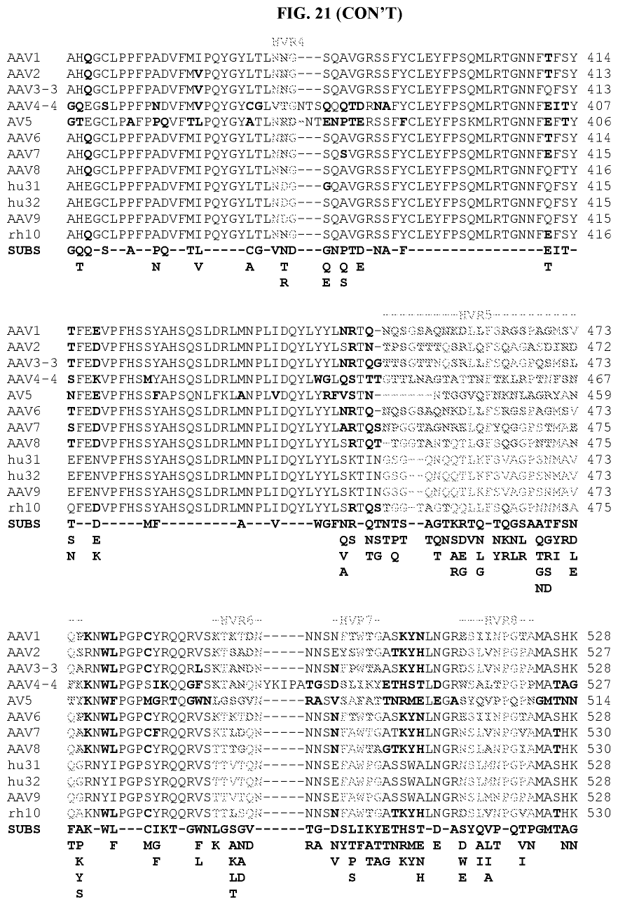

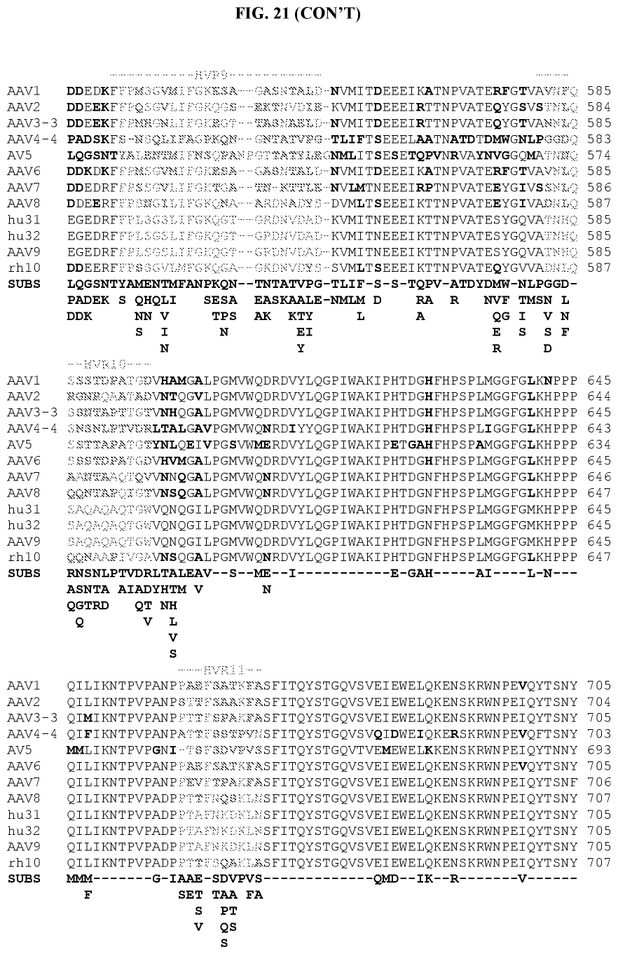

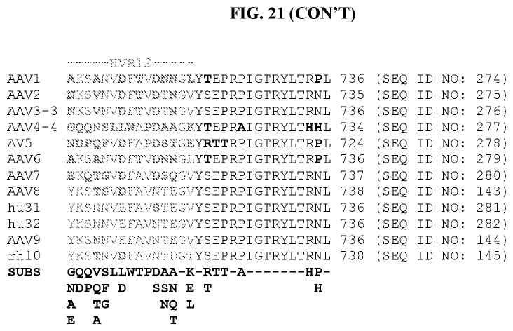

In certain embodiments, the viral vectors provided herein are AAV based viral vectors. In certain embodiments, the AAV-based vectors provided herein do not encode the AAV rep gene (required for replication) and/or the AAV cap gene (required for synthesis of the capsid proteins) (the rep and cap proteins may be provided by the packaging cells in trans). Multiple AAV serotypes have been identified. In certain embodiments, AAV-based vectors provided herein comprise components from one or more serotypes of AAV. In certain embodiments, AAV based vectors provided herein comprise capsid components from one or more of AAV1 (SEQ ID NO: 274), AAV2 (SEQ ID NO: 275), AAV2.7m8 (SEQ ID NO: 142), AAV3 (SEQ ID NO: 276), AAV4 (SEQ ID NO: 277), AAV5, AAV6 (SEQ ID NO: 279), AAV7 (SEQ ID NO: 280), AAV8 (SEQ ID NO: 143), AAV9 (SEQ ID NO: 144), AAV10, AAV11, or AAVrh10 (SEQ ID NO: 145). In certain embodiments, AAV based vectors provided herein comprise components from one or more of AAV8, AAV9, AAV10, AAV11, or AAVrh10 serotypes. Provided are viral vectors in which the capsid protein is a variant of the AAV8 capsid protein (SEQ ID NO: 143), AAV9 capsid protein (SEQ ID NO: 144), or AAVrh10 capsid protein (SEQ ID NO: 145), and the capsid protein is e.g., at least 95%, 96%, 97%, 98%, 99% or 99.9% identical to the amino acid sequence of the AAV8 capsid protein (SEQ ID NO: 143), AAV9 capsid protein (SEQ ID NO: 144), or AAVrh10 capsid protein (SEQ ID NO: 145), while retaining the biological function of the native capsid. In certain embodiments, the encoded AAV capsid has the sequence of SEQ ID NO: 143, 144, or 145 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8 AAV9 or AAVrh10 capsid. FIG. 21 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV vector comprises an AAV8, AAV9 or AAVrh10 capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions that are not present at that position in the native AAV capsid sequence as identified in the SUBS row of FIG. 21. Amino acid sequence for AAV8, AAV9, and AAVrh10 capsids are provided in FIG. 21.

In some embodiments, AAV-based vectors comprise components from one or more serotypes of AAV. In some embodiments, AAV based vectors provided herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof. In some embodiments, AAV based vectors provided herein comprise components from one or more of AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC 10, AAV.HSC11, AAV.HSC12, AAV.HSC13, AAV.HSC14, AAV.HSC15, or AAV.HSC16 or other rAAV particles, or combinations of two or more thereof serotypes. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof.

In particular embodiments, the recombinant AAV for use in compositions and methods herein is Anc80 or Anc80L65 (see, e.g., Zinn et al., 2015, Cell Rep. 12(6): 1056-1068, which is incorporated by reference in its entirety). In particular embodiments, the recombinant AAV for use in compositions and methods herein is AAV.7m8 (including variants thereof) (see, e.g., U.S. Pat. Nos. 9,193,956; 9,458,517; 9,587,282; US 2016/0376323, and WO 2018/075798, each of which is incorporated herein by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLoS One 8(4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

AAV8-based, AAV9-based, and AAVrh10-based viral vectors are used in certain of the methods described herein. Nucleotide sequences of AAV based viral vectors and methods of making recombinant AAV and AAV capsids are taught, for example, in U.S. Pat. Nos. 7,282,199 B2, 7,790,449 B2, 8,318,480 B2, 8,962,332 B2 and International Patent Application No. PCT/EP2014/076466, each of which is incorporated herein by reference in its entirety. In one aspect, provided herein are AAV (e.g., AAV8, AAV9 or AAVrh10)-based viral vectors encoding a transgene (e.g., an HuPTM Fab). The amino acid sequences of AAV capsids, including AAV8, AAV9 and AAVrh10 are provided in FIG. 21.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the transgene encoding the HuPTMmAb or HuGlyFab or antigen-binding fragment. The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the transgene encoding the HuPTM mAb antigen binding fragment. Four plasmids are used to make the construct: Gag/pol sequence containing plasmid, Rev sequence containing plasmids, Envelope protein containing plasmid (e.g., VSV-G), and Cis plasmid with the packaging elements and the anti-VEGF antigen-binding fragment gene.

For lentiviral vector production, the four plasmids are co-transfected into cells (e.g., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes an HuPTM mAb antigen binding fragment, such as an HuGlyFab, such that, upon introduction of the vector into a relevant cell, a glycosylated and/or tyrosine sulfated variant of the HuPTM mAb antigen binding fragment or HuGlyFab is expressed by the cell.

### 5.1.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters that control expression of the transgene. In certain embodiments, the promoter is a constitutive promoter. In certain embodiments, the promoter is a CB7 (also referred to as a CAG promoter) (see Dinculescu et al., 2005, Hum Gene Ther 16: 649-663, incorporated by reference herein in its entirety). In some embodiments, the CAG or CB7 promoter (SEQ ID NO: 411) includes other expression control elements that enhance expression of the transgene driven by the vector. In certain embodiments, the other expression control elements include chicken β-actin intron and/or rabbit β-globin polyA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs. In certain embodiments, the vectors provided herein comprise one or more tissue specific promoters (e.g., a retinal pigment epithelial cell-specific promoter, a CNS-specific promoter, a liver-specific promoter or muscle specific). In certain embodiments, the viral vectors provided herein comprise a RPE65 promoter or an opsin promoter (a retinal cell/CNS specific promoter). In certain embodiments, the viral vectors provided herein comprises a liver cell specific promoter, such as, a TBG (Thyroxine-binding Globulin) promoter (SEQ ID NO: 423), an APOA2 promoter, a SERPINA1 (hAAT) promoter, an ApoE.hAAT promoter (SEQ ID NO: 412), or a MIR122 promoter. In certain embodiments, the viral vector provided herein comprises a muscle specific promoter, such as a human desmin promoter (Jonuschies et al., 2014, Curr. Gene Ther. 14:276-288), a CK8 promoter (SEQ ID NO: 413; Himeda et al., 2011 Muscle Gene Therapy: Methods and Protocols, Methods in Molecular Biology, Dongsheng Duan (ed.), 709:3-19; SEQ ID NO: 413), or a Pitx3 promoter (Coulon et al., 2007, JBC 282:33192). In other embodiments, the viral vector comprises a VMD2 promoter. In certain embodiments, the viral vector herein comprises synthetic and tandem promoters, e.g. the promoters listed in Table 1 below.

In certain embodiments, the promoter is an inducible promoter. In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2α binding site. In certain embodiments, the HIF binding site comprises an RCGTG motif. For details regarding the location and sequence of HIF binding sites, see, e.g., Schödel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety. In specific embodiments, the hypoxia-inducible promoter is the human N-WASP promoter, see, e.g., Salvi, 2017, Biochemistry and Biophysics Reports 9:13-21 (incorporated by reference for the teaching of the N-WASP promoter) or is the hypoxia-induced promoter of human Epo, see, e.g., Tsuchiya et al., 1993, J. Biochem. 113:395-400 (incorporated by reference for the disclosure of the Epo hypoxia-inducible promoter). In other embodiments, the promoter is a drug inducible promoter, for example, a promoter that is induced by administration of rapamycin or analogs thereof. See, e.g., the disclosure of rapamycin inducible promoters in PCT publications WO94/18317, WO 96/20951, WO 96/41865, WO 99/10508, WO 99/10510, WO 99/36553, and WO 99/41258, and U.S. Pat. No. 7,067,526, which are hereby incorporated by reference in their entireties for the disclosure of drug inducible promoters.

Provided herein are constructs containing certain ubiquitous and tissue specific promoters. Such promoters include synthetic and tandem promoters. Examples and nucleotide sequences of promoters are provided in Table 1 below.

TABLE 1
Promoter and Other Regulatory Element Sequences

| Name/ | |
|---|---|
| SEQ ID NO: | Sequence |
| LSPX1 | aggttaatttttaaaaagcagtcaaaagtccaagtggcccttggcagcatttactctctctgt |
| SEQ ID NO: 315 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgtctagaaggctc |
| | agaggcacacaggagtttctgggctcaccctgcccccttccaacccctcagttcccatcctcc |
| | agcagctgtttgtgtgctgcctctgaagtccacactgaacaaacttcagcctactcatgtccc |
| | taaaatgggcaaacattgcaagcagcaaacagcaaacacacagccctccctgcctgctgacct |
| | tggagctggggcagaggtcagagacctctctgggcccatgccacctccaacatccactcgacc |
| | ccttggaatttcggtggagaggagcagaggttgtcctggcgtggtttaggtagtgtgagaggg |
| | gtacccggggatcttgctaccagtggaacagccactaaggattctgcagtgagagcagagggc |
| | cagctaagtggtactctcccagagactgtctgactcacgccaccccctccaccttggacacag |
| | gacgctgtggtttctgagccaggtacaatgactcctttcggtaagtgcagtggaagctgtaca |
| | ctgcccaggcaaagcgtccgggcagcgtaggcgggcgactcagatcccagccagtggacttag |
| | cccctgtttgctcctccgataactggggtgaccttggttaatattcaccagcagcctcccccg |
| | ttgcccctctggatccactgcttaaatacggacgaggacagggccctgtctcctcagcttcag |
| | gcaccaccactgacctgggacagt |
| LSXP2 | aggctcagaggcacacaggagtttctgggctcaccctgcccocttccaacccctcagttccca |
| SEQ ID NO: 316 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagaaggctcagaggcacacaggagtttctgggctcaccctgcccccttccaacc |
| | cctcagttcccatcctccagcagctgtttgtgtgctgcctctgaagtccacactgaacaaact |
| | tcagcctactcatgtccctaaaatgggcaaacattgcaagcagcaaacagcaaacacacagcc |
| | ctccctgcctgctgaccttggagctggggcagaggtcagagacctctctgggcccatgccacc |
| | tccaacatccactcgaccccttggaatttcggtggagaggagcagaggttgtcctggcgtggt |
| | ttaggtagtgtgagaggggtacccggggatcttgctaccagtggaacagccactaaggattct |
| | gcagtgagagcagagggccagctaagtggtactctcccagagactgtctgactcacgccaccc |
| | cctccaccttggacacaggacgctgtggtttctgagccaggtacaatgactcctttcggtaag |
| | tgcagtggaagctgtacactgcccaggcaaagcgtccgggcagcgtaggcgggcgactcagat |
| | cccagccagtggacttagcccctgtttgctcctccgataactggggtgaccttggttaatatt |
| | caccagcagcctcccccgttgcccctctggatccactgcttaaatacggacgaggacagggcc |
| | ctgtctcctcagcttcaggcaccaccactgacctgggacagt |
| LTP1 | aggttaatttttaaaaagcagtcaaaagtccaagtggcccttggcagcatttactctctctgt |
| SEQ ID NO: 317 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgacatcatttcct |
| | ctgcgaatgcatgtataatttctacagaacctattagaaaggatcacccagcctctgcttttg |
| | tacaactttcccttaaaaaactgccaattccactgctgtttggcccaatagtgagaacttttt |
| | cctgctgcctcttggtgcttttgcctatggcccctattctgcctgctgaagacactcttgcca |
| | gcatggacttaaacccctccagctctgacaatcctctttctcttttgttttacatgaagggtc |
| | tggcagccaaagcaatcactcaaagttcaaaccttatcattttttgctttgttcctcttggcc |
| | ttggttttgtacatcagctttgaaaataccatcccagggttaatgctggggttaatttataac |
| | taagagtgctctagttttgcaatacaggacatgctataaaaatggaaagatgttgctttctga |
| | gaggatcttgctaccagtggaacagccactaaggattctgcagtgagagcagagggccagcta |
| | agtggtactctcccagagactgtctgactcacgccaccccctccaccttggacacaggacgct |
| | gtggtttctgagccaggtacagtgactcctttcggtaagtgcagtggaagctgtacactgccc |
| | aggcaaagcgtccgggcagcgtaggcgggcgactcagatcccagccagtggacttagcccctg |
| | tttgctcctccgataactggggtgaccttggttaatattcaccagcagcctcccccgttgccc |
| | ctctggatccactgcttaaatacggacgaggacagggccctgtctcctcagcttcaggcacca |
| | ccactgacctgggacagt |
| LTP2 | aggctcagaggcacacaggagtttctgggctcaccctgcccccttccaacccctcagttccca |
| SEQ ID NO: 318 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagagcccttaagctagcaggttaatttttaaaaagcagtcaaaagtccaagtgg |
| | cccttggcagcatttactctctctgtttgctctggttaataatctcaggagcacaaacattcc |
| | agatccaggttaatttttaaaaagcagtcaaaagtccaagtggcccttggcagcatttactct |
| | ctctgtttgctctggttaataatctcaggagcacaaacattccagatccggcgcgccagggct |
| | ggaagctacctttgacatcatttcctctgcgaatgcatgtataatttctacagaacctattag |
| | aaaggatcacccagcctctgcttttgtacaactttcccttaaaaaactgccaattccactgct |
| | gtttggcccaatagtgagaactttttcctgctgcctcttggtgcttttgcctatggcccctat |
| | tctgcctgctgaagacactcttgccagcatggacttaaacccctccagctctgacaatcctct |
| | ttctcttttgttttacatgaagggtctggcagccaaagcaatcactcaaagttcaaaccttat |
| | cattttttgctttgttcctcttggccttggttttgtacatcagctttgaaaataccatcccag |
| | ggttaatgctggggttaatttataactaagagtgctctagttttgcaatacaggacatgctat |
| | aaaaatggaaagatgttgctttctgagaggatcttgctaccagtggaacagccactaaggatt |
| | ctgcagtgagagcagagggccagctaagtggtactctcccagagactgtctgactcacgccac |
| | cccctccaccttggacacaggacgctgtggtttctgagccaggtacagtgactcctttcggta |
| | agtgcagtggaagctgtacactgcccaggcaaagcgtccgggcagcgtaggcgggcgactcag |
| | atcccagccagtggacttagcccctgtttgctcctccgataactggggtgaccttggttaata |
| | ttcaccagcagcctcccccgttgcccctctggatccactgcttaaatacggacgaggacaggg |
| | ccctgtctcctcagcttcaggcaccaccactgacctgggacagt |
| LTP3 | aggttaatttttaaaaagcagtcaaaagtccaagtggcccttggcagcatttactctctctgt |
| SEQ ID NO: 319 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgacatcatttcct |
| | ctgcgaatgcatgtataatttctacagaacctattagaaaggatcacccagcctctgcttttg |
| | tacaactttcccttaaaaaactgccaattccactgctgtttggcccaatagtgagaacttttt |
| | cctgctgcctcttggtgcttttgcctatggcccctattctgcctgctgaagacactcttgcca |
| | gcatggacttaaacccctccagctctgacaatcctctttctcttttgttttacatgaagggtc |
| | tggcagccaaagcaatcactcaaagttcaaaccttatcattttttgctttgttcctcttggcc |
| | ttggttttgtacatcagctttgaaaataccatcccagggttaatgctggggttaatttataac |
| | taagagtgctctagttttgcaatacaggacatgctataaaaatggaaagatgttgctttctga |
| | gaggatcttgctaccagtggaacagccactaaggattctgcagtgagagcagagggccagcta |
| | agtggtactctcccagagactgtctgactcacgccaccccctccaccttggacacaggacgct |
| | gtggtttctgagccaggtacagtgactcctttcggtaagtgcagtggaagctgtacactgccc |
| | aggcaaagcgtccgggcagcgtaggcgggcgactcagatcccagccagtggacttagcccctg |
| | tttgctcctccgataactggggtgaccttggttaatattcaccagcagcctcccccgttgccc |
| | ctctggatccactgcttaaatacggacgaggacagggccctgtctcctcagcttcaggcacca |
| | ccactgacctgggacagtaaaacaggtaagtccgctgtttgtgtgctgcctctgaagtccaca |
| | ctgaacaaacttcagcctactcatgtccctaaaatgggcaaacattgcaagcagcaaacagca |
| | aacacacagccctccctgcctgctgaccttggagctggggcagaggtcagagacctctctggc |
| | ctctactaaccatgttcatgttttctttttttttctacaggtcctgggtgacgaacag |
| LMTP6 | aggctcagaggcacacaggagtttctgggctcaccctgcccccttccaacccctcagttccca |
| SEQ ID NO: 320 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccactacgggtttaggctgcccatgtaaggaggcaaggcctggggacacccgagatg |
| | cctggttataattaacccagacatgtggctgccccccccccccccaacacctgctgcctctaa |
| | aaataaccctgtccctggtggatcccactacgggtttaggctgcccatgtaaggaggcaaggc |
| | ctggggacacccgagatgcctggttataattaacccagacatgtggctgcccccccccccccc |
| | aacacctgctgcctctaaaaataaccctgtccctggtggatcccactacgggtttaggctgcc |
| | catgtaaggaggcaaggcctggggacacccgagatgcctggttataattaacccagacatgtg |
| | gctgccccccccccccccaacacctgctgcctctaaaaataaccctgtccctggtggatcccc |
| | tgcatgcgaagatcttcgaacaaggctgtgggggactgagggcaggctgtaacaggcttgggg |
| | gccagggcttatacgtgcctgggactcccaaagtattactgttccatgttcccggcgaagggc |
| | cagctgtcccccgccagctagactcagcacttagtttaggaaccagtgagcaagtcagccctt |
| | ggggcagcccatacaaggccatggggctgggcaagctgcacgcctgggtccggggtgggcacg |
| | gtgcccgggcaacgagctgaaagctcatctgctctcaggggcccctccctggggacagcccct |
| | cctggctagtcacaccctgtaggctcctctatataacccaggggcacaggggctgccctcatt |
| | ctaccaccacctccacagcacagacagacactcaggagccagccagcgtcgagatcttgctac |
| | cagtggaacagccactaaggattctgcagtgagagcagagggccagctaagtggtactctccc |
| | agagactgtctgactcacgccaccccctccaccttggacacaggacgctgtggtttctgagcc |
| | aggtacagtgactcctttcggtaagtgcagtggaagctgtacactgcccaggcaaagcgtccg |
| | ggcagcgtaggcgggcgactcagatcccagccagtggacttagcccctgtttgctcctccgat |
| | aactggggtgaccttggttaatattcaccagcagcctcccccgttgcccctctggatccactg |
| | cttaaatacggacgaggacagggccctgtctcctcagcttcaggcaccaccactgacctggga |
| | cagt |
| LMTP13 | aggctcagaggcacacaggagtttctgggctcaccctgcccccttccaacccctcagttccca |
| SEQ ID NO: 321 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agaggggtacccggggatcttgctaccagtctagaggccgtccgccctcggcaccatcctcac |
| | gacacccaaatatggcgacgggtgaggaatggtggggagttatttttagagcggtgaggaagg |
| | tgggcaggcagcaggtgttggcgctctaaaaataactcccgggagttatttttagagcggagg |
| | aatggtggacacccaaatatggcgacggttcctcacccgtcgccatatttgggtgtccgccct |
| | cggccggggccgcattcctgggggccgggcggtgctcccgcccgcctcgataaaaggctccgg |
| | ggccggcggcggcccacgagctacccggaggagcgggaggcgccaagcgtgagtatcgatctt |
| | gctaccagtggaacagccactaaggattctgcagtgagagcagagggccagctaagtggtact |
| | ctcccagagactgtctgactcacgccaccccctccaccttggacacaggacgctgtggtttct |
| | gagccaggtacagtgactcctttcggtaagtgcagtggaagctgtacactgcccaggcaaagc |
| | gtccgggcagcgtaggcgggcgactcagatcccagccagtggacttagcccctgtttgctcct |
| | ccgataactggggtgaccttggttaatattcaccagcagcctcccccgttgcccctctggatc |
| | cactgcttaaatacggacgaggacagggccctgtctcctcagcttcaggcaccaccactgacc |
| | tgggacagt |
| LMTP14 | gaatggtggacacccaaatatggcgacggttcctcacccgtcgccatatttgggtgtccgccc |
| SEQ ID NO: 322 | tcggccggggccgcattcctgggggccgggcggtgctcccgcccgcctcgataaaaggctccg |
| | gggccggcggcggcccacgagctacccggaggagcgggaggcgccaagcgatcttgctaccag |
| | tggaacagccactaaggattctgcagtgagagcagagggccagctaagtggtactctcccaga |
| | gactgtctgactcacgccaccccctccaccttggacacaggacgctgtggtttctgagccagg |
| | tacagtgactcctttcggtaagtgcagtggaagctgtacactgcccaggcaaagcgtccgggc |
| | agcgtaggcgggcgactcagatcccagccagtggacttagcccctgtttgctcctccgataac |
| | tggggtgaccttggttaatattcaccagcagcctcccccgttgcccctctggatccactgctt |
| | aaatacggacgaggacagggccctgtctcctcagcttcaggcaccaccactgacctgggacag |
| | t |
| LMTP15 | aggctcagaggcacacaggagtttctgggctcaccctgcccccttccaacccctcagttccca |
| SEQ ID NO: 323 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagagaatggtggacacccaaatatggcgacggttcctcacccgtcgccatattt |
| | gggtgtccgccctcggccggggccgcattcctgggggccgggcggtgctcccgcccgcctcga |
| | taaaaggctccggggccggcggcggcccacgagctacccggaggagcgggaggcgccaagcga |
| | tcttgctaccagtggaacagccactaaggattctgcagtgagagcagagggccagctaagtgg |
| | tactctcccagagactgtctgactcacgccaccccctccaccttggacacaggacgctgtggt |
| | ttctgagccaggtacagtgactcctttcggtaagtgcagtggaagctgtacactgcccaggca |
| | aagcgtccgggcagcgtaggcgggcgactcagatcccagccagtggacttagcccctgtttgc |
| | tcctccgataactggggtgaccttggttaatattcaccagcagcctcccccgttgcccctctg |
| | gatccactgcttaaatacggacgaggacagggccctgtctcctcagcttcaggcaccaccact |
| | gacctgggacagt |
| LMTP18 | aggctcagaggcacacaggagtttctgggctcaccctgcccccttccaacccctcagttccca |
| SEQ ID NO: 324 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccactacgggtttaggctgcccatgtaaggaggcaaggcctggggacacccgagatg |
| | cctggttataattaacccagacatgtggctgccccccccccccccaacacctgctgcctctaa |
| | aaataaccctgtccctggtggatcccctgcatgcgaagatcttcgaacaaggctgtgggggac |
| | tgagggcaggctgtaacaggcttgggggccagggcttatacgtgcctgggactcccaaagtat |
| | tactgttccatgttcccggcgaagggccagctgtcccccgccagctagactcagcacttagtt |
| | taggaaccagtgagcaagtcagcccttggggcagcccatacaaggccatggggctgggcaagc |
| | tgcacgcctgggtccggggtgggcacggtgcccgggcaacgagctgaaagctcatctgctctc |
| | aggggcccctccctggggacagcccctcctggctagtcacaccctgtaggctcctctatataa |
| | cccaggggcacaggggctgccctcattctaccaccacctccacagcacagacagacactcagg |
| | agccagccagcgtcgagatcttgctaccagtggaacagccactaaggattctgcagtgagagc |
| | agagggccagctaagtggtactctcccagagactgtctgactcacgccaccccctccaccttg |
| | gacacaggacgctgtggtttctgagccaggtacagtgactcctttcggtaagtgcagtggaag |
| | ctgtacactgcccaggcaaagcgtccgggcagcgtaggcgggcgactcagatcccagccagtg |
| | gacttagcccctgtttgctcctccgataactggggtgaccttggttaatattcaccagcagcc |
| | tcccccgttgcccctctggatccactgcttaaatacggacgaggacagggccctgtctcctca |
| | gcttcaggcaccaccactgacctgggacagt |
| LMTP19 | aggctcagaggcacacaggagtttctgggctcaccctgcccccttccaacccctcagttccca |
| SEQ ID NO: 325 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccctgcatgcgaagatcttcgaacaaggctgtgggggactgagggcaggctgtaaca |
| | ggcttgggggccagggcttatacgtgcctgggactcccaaagtattactgttccatgttcccg |
| | gcgaagggccagctgtcccccgccagctagactcagcacttagtttaggaaccagtgagcaag |
| | tcagcccttggggcagcccatacaaggccatggggctgggcaagctgcacgcctgggtccggg |
| | gtgggcacggtgcccgggcaacgagctgaaagctcatctgctctcaggggcccctccctgggg |
| | acagcccctcctggctagtcacaccctgtaggctcctctatataacccaggggcacaggggct |
| | gccctcattctaccaccacctccacagcacagacagacactcaggagccagccagcgtcgaga |
| | tcttgctaccagtggaacagccactaaggattctgcagtgagagcagagggccagctaagtgg |
| | tactctcccagagactgtctgactcacgccaccccctccaccttggacacaggacgctgtggt |
| | ttctgagccaggtacagtgactcctttcggtaagtgcagtggaagctgtacactgcccaggca |
| | aagcgtccgggcagcgtaggcgggcgactcagatcccagccagtggacttagcccctgtttgc |
| | tcctccgataactggggtgaccttggttaatattcaccagcagcctcccccgttgcccctctg |
| | gatccactgcttaaatacggacgaggacagggccctgtctcctcagcttcaggcaccaccact |
| | gacctgggacagt |
| LMTP20 | aggctcagaggcacacaggagtttctgggctcaccctgcccccttccaacccctcagttccca |
| SEQ ID NO: 326 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggcccttcagattaaaaataactgaggtaagggcctgggtaggggaggtggtgtgagac |
| | gctcctgtctctcctctatctgcccatcggccctttggggaggaggaatgtgcccaaggacta |
| | aaaaaaggccatggagccagaggggcgagggcaacagacctttcatgggcaaaccttggggcc |
| | ctgctgaagctttggcccactacgggtttaggctgcccatgtaaggaggcaaggcctggggac |
| | acccgagatgcctggttataattaacccagacatgtggctgccccccccccccccaacacctg |
| | ctgcctctaaaaataaccctgtccctggtggatcccctgcatgcgaagatcttcgaacaaggc |
| | tgtgggggactgagggcaggctgtaacaggcttgggggccagggcttatacgtgcctgggact |
| | cccaaagtattactgttccatgttcccggcgaagggccagctgtcccccgccagctagactca |
| | gcacttagtttaggaaccagtgagcaagtcagcccttggggcagcccatacaaggccatgggg |
| | ctgggcaagctgcacgcctgggtccggggtgggcacggtgcccgggcaacgagctgaaagctc |
| | atctgctctcaggggcccctccctggggacagcccctcctggctagtcacaccctgtaggctc |
| | ctctatataacccaggggcacaggggctgccctcattctaccaccacctccacagcacagaca |
| | gacactcaggagccagccagcgtcgagatcttgctaccagtggaacagccactaaggattctg |
| | cagtgagagcagagggccagctaagtggtactctcccagagactgtctgactcacgccacccc |
| | ctccaccttggacacaggacgctgtggtttctgagccaggtacagtgactcctttcggtaagt |
| | gcagtggaagctgtacactgcccaggcaaagcgtccgggcagcgtaggcgggcgactcagatc |
| | ccagccagtggacttagcccctgtttgctcctccgataactggggtgaccttggttaatattc |
| | accagcagcctcccccgttgcccctctggatccactgcttaaatacggacgaggacagggccc |
| | tgtctcctcagcttcaggcaccaccactgacctgggacagt |
| LBTP1 | aggttaatttttaaaaagcagtcaaaagtccaagtggcccttggcagcatttactctctctgt |
| SEQ ID NO: 327 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgtctagaaggctc |
| | agaggcacacaggagtttctgggctcaccctgcccccttccaacccctcagttcccatcctcc |
| | agcagctgtttgtgtgctgcctctgaagtccacactgaacaaacttcagcctactcatgtccc |
| | taaaatgggcaaacattgcaagcagcaaacagcaaacacacagccctccctgcctgctgacct |
| | tggagctggggcagaggtcagagacctctctgggcccatgccacctccaacatccactcgacc |
| | ccttggaatttcggtggagaggagcagaggttgtcctggcgtggtttaggtagtgtgagaggg |
| | gtacccggggatcttgctaccagtggaacagccactaaggattctgcagtgagagcagagggc |
| | cagctaagtggtactctcccagagactgtctgactcacgccaccccctccaccttggacacag |
| | gacgctgtggtttctgagccaggtacaatgactcctttcggtaagtgcagtggaagctgtaca |
| | ctgcccaggcaaagcgtccgggcagcgtaggcgggcgactcagatcccagccagtggacttag |
| | cccctgtttgctcctccgataactggggtgaccttggttaatattcaccagcagcctcccccg |
| | ttgcccctctggatccactgcttaaatacggacgaggacagggccctgtctcctcagcttcag |
| | gcaccaccactgacctgggacagt |
| LBTP2 | aggctcagaggcacacaggagtttctgggctcaccctgcccccttccaacccctcagttccca |
| SEQ ID NO: 328 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggcacacatacacgaacacacatacatatacatacattcacatatatgcacacacatac |
| | acatatacacgcatacacgtacacacaaatgcacactcacacatgcacacacatacataatat |
| | acacactctcacacatgcacatacacacatacatacacatacatgtgcatgcacacacacaaa |
| | tacacatgcatacatccacattcacacagatgcagacacaaatgcacacacacacacacacac |
| | acacacacacacacacacacacacgcacactgccaccctgaactagtggtggctaaatgaaca |
| | ataagtctccatcaccagcttggggggaggtaggtggtagtgtaggtgcccccattgtgtgat |
| | catgttcattgtatgagtttgtctgtgttcattcatcatagtgacagtccccatgtgggtagc |
| | agagagtacgtgtgcatgcatcatctccgtgtttgctcatgagtgtgtatgtcagtgtgttcc |
| | agtctttctgtgtgagtgtcgtccccaatcccccatccccccccccagatctctaattagtgg |
| | tttggggtttgttccttttccctcctgttcctttcctcagcagcgcggcagcagcggcggcag |
| | cctcggtggtagcagcagcagcagcagcagcagcagcagcagcagcagcagcagcagcaacag |
| | aagctgccgcgccgctgagtagcagcaaggactccgagtcaagagtaggattgtaggattgga |
| | tctgagtgggaacaagagtgagctggcctgagagaggagcagatgcctcccagcgccctcagg |
| | ccacccattgccagtaatcttcaagccagacctcttgagaggagacgggacagccaaccctag |
| | cctacccaggtacagacactgggcagttctgggggactgcccacagatgcctattggattcct |
| | ggggtatgtaggactcccgggtctaccagcccttttcacctttccccatagcacccccaagga |
| | agctctgacaacttgcccatattcctgtttcccacccgtcccctgggcacccccttttcttct |
| | ctccctcccagatcccttctttggggagctcagcaaatggagcaggaaatttggaccctctgc |
| | ctccctctctcgccttcctcattggatccggagtcttctccgctgggaaagctgtaattagag |
| | ggtggatccctacagacagagagcagcccccccacccccaccccccagtcccttctaacttta |
| | gatctcttctctcccattctcccattctccctccctctcccttctccctctcccactggctcc |
| | tcggttctctccatctgcctgactccttgggacccggtccccagatcttgctaccagtggaac |
| | agccactaaggattctgcagtgagagcagagggccagctaagtggtactctcccagagactgt |
| | ctgactcacgccaccccctccaccttggacacaggacgctgtggtttctgagccaggtacagt |
| | gactcctttcggtaagtgcagtggaagctgtacactgcccaggcaaagcgtccgggcagcgta |
| | ggcgggcgactcagatcccagccagtggacttagcccctgtttgctcctccgataactggggt |
| | gaccttggttaatattcaccagcagcctcccccgttgcccctctggatccactgcttaaatac |
| | ggacgaggacagggccctgtctcctcagcttcaggcaccaccactgacctgggacagt |
| SP7 | cacacatacacgaacacacatacatatacatacattcacatatatgcacacacatacacatat |
| SEQ ID NO: 329 | acacgcatacacgtacacacaaatgcacactcacacatgcacacacatacataatatacacac |
| | tctcacacatgcacatacacacatacatacacatacatgtgcatgcacacacacaaatacaca |
| | tgcatacatccacattcacacagatgcagacacaaatgcacacacacacacacacacacacac |
| | acacacacacacacacacgcacactgccaccctgaactagtggtggctaaatgaacaataagt |
| | ctccatcaccagcttggggggaggtaggtggtagtgtaggtgcccccattgtgtgatcatgtt |
| | cattgtatgagtttgtctgtgttcattcatcatagtgacagtccccatgtgggtagcagagag |
| | tacgtgtgcatgcatcatctccgtgtttgctcatgagtgtgtatgtcagtgtgttccagtctt |
| | tctgtgtgagtgtcgtccccaatcccccatccccccccccagatctctaattagtggtttggg |
| | gtttgttccttttccctcctgttcctttcctcagcagcgcggcagcagcggcggcagcctcgg |
| | tggtagcagcagcagcagcagcagcagcagcagcagcagcagcagcagcagcaacagaagctg |
| | ccgcgccgctgagtagcagcaaggactccgagtcaagagtaggattgtaggattggatctgag |
| | tgggaacaagagtgagctggcctgagagaggagcagatgcctcccagcgccctcaggccaccc |
| | attgccagtaatcttcaagccagacctcttgagaggagacgggacagccaaccctagcctacc |
| | caggtacagacactgggcagttctgggggactgcccacagatgcctattggattcctggggta |
| | tgtaggactcccgggtctaccagcccttttcacctttccccatagcacccccaaggaagctct |
| | gacaacttgcccatattcctgtttcccacccgtcccctgggcacccccttttcttctctccct |
| | cccagatcccttctttggggagctcagcaaatggagcaggaaatttggaccctctgcctccct |
| | ctctcgccttcctcattggatccggagtcttctccgctgggaaagctgtaattagagggtgga |
| | tccctacagacagagagcagcccccccacccccaccccccagtcccttctaactttagatctc |
| | ttctctcccattctcccattctccctccctctcccttctccctctcccactggctcctcggtt |
| | ctctccatctgcctgactccttgggacccggtcccca |
| minSP7 | cgcggcagcagcggcggcagcctcggtggtagcagcagcagcagcagcagcagcagcagcagc |
| SEQ ID NO: 330 | agcagcagcagcagcaacagaagctgccgcgccgctgagtagcagcaaggactccgagtcaag |
| | agtaggattgtaggattggatctgagtgggaacaagagtgagctggcctgagagaggagcaga |
| | tgcctcccagcgccctcaggccacccattgccagtaatcttcaagccagacctcttgagagga |
| | gacgggacagccaaccctagcctacccaggtacagacactgggcagttctgggggactgccca |
| | cagatgcctattggattcctggggtatgtaggactcccgggtctaccagcccttttcaccttt |
| | ccccatagcacccccaaggaagctctgacaacttgcccatattcctgtttcccacccgtcccc |
| | tgggcacccccttttcttctctccctcccagatcccttctttggggagctcagcaaatggagc |
| | aggaaatttggaccctctgcctccctctctcgccttcctcattggatccggagtcttctccgc |
| | tgggaaagctgtaattagagggtggatccctacagacagagagcagcccccccacccccaccc |
| | cccagtcccttctaactttagatctcttctctcccattctcccattctccctccctctccctt |
| | ctccctctcccactggctcctcggttctctccatctgcctgactccttgggacccggtcccca |
| ApoE.hAAT | aggctcagaggcacacaggagtttctgggctcaccctgcccccttccaacccctcagttccca |
| SEQ ID NO: 412 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agaggggtacccggggatcttgctaccagtggaacagccactaaggattctgcagtgagagca |
| | gagggccagctaagtggtactctcccagagactgtctgactcacgccaccccctccaccttgg |
| | acacaggacgctgtggtttctgagccaggtacaatgactcctttcggtaagtgcagtggaagc |
| | tgtacactgcccaggcaaagcgtccgggcagcgtaggcgggcgactcagatcccagccagtgg |
| | acttagcccctgtttgctcctccgataactggggtgaccttggttaatattcaccagcagcct |
| | cccccgttgcccctctggatccactgcttaaatacggacgaggacagggccctgtctcctcag |
| | cttcaggcaccaccactgacctgggacagt |
| CAG/CB7 | gacattgattattgactagttattaatagtaatcaattacggggtcattagttcatagcccat |
| SEQ ID NO: 411 | atatggagttccgcgttacataacttacggtaaatggcccgcctggctgaccgcccaacgacc |
| | cccgcccattgacgtcaataatgacgtatgttcccatagtaacgccaatagggactttccatt |
| | gacgtcaatgggtggagtatttacggtaaactgcccacttggcagtacatcaagtgtatcata |
| | tgccaagtacgccccctattgacgtcaatgacggtaaatggcccgcctggcattatgcccagt |
| | acatgaccttatgggactttcctacttggcagtacatctacgtattagtcatcgctattacca |
| | tggtcgaggtgagccccacgttctgcttcactctccccatctcccccccctccccacccccaa |
| | ttttgtatttatttattttttaattattttgtgcagcgatgggggcggggggggggggggggc |
| | gcgcgccaggcggggcggggcggggcgaggggcggggcggggcgaggcggagaggtgcggcgg |
| | cagccaatcagagcggcgcgctccgaaagtttccttttatggcgaggcggcggcggcggcggc |
| | cctataaaaagcgaagcgcgcggcgggcgggagtcgctgcgcgctgccttcgccccgtgcccc |
| | gctccgccgccgcctcgcgccgcccgccccggctctgactgaccgcgttactcccacaggtga |
| | gcgggcgggacggcccttctcctccgggctgtaattagcgcttggtttaatgacggcttgttt |
| | cttttctgtggctgcgtgaaagccttgaggggctccgggagggccctttgtgcggggggagcg |
| | gctcggggggtgcgtgcgtgtgtgtgtgcgtggggagcgccgcgtgcggctccgcgctgcccg |
| | gcggctgtgagcgctgcgggcgcggcgcggggctttgtgcgctccgcagtgtgcgcgagggga |
| | gcgcggccgggggcggtgccccgcggtgcggggggggctgcgaggggaacaaaggctgcgtgc |
| | ggggtgtgtgcgtgggggggtgagcagggggtgtgggcgcgtcggtcgggctgcaaccccccc |
| | tgcacccccctccccgagttgctgagcacggcccggcttcgggtgcggggctccgtacggggc |
| | gtggcgcggggctcgccgtgccgggcggggggtggcggcaggtgggggtgccgggcggggcgg |
| | ggccgcctcgggccggggagggctcgggggaggggcgcggcggcccccggagcgccggcggct |
| | gtcgaggcgcggcgagccgcagccattgccttttatggtaatcgtgcgagagggcgcagggac |
| | ttcctttgtcccaaatctgtgcggagccgaaatctgggaggcgccgccgcaccccctctagcg |
| | ggcgcggggcgaagcggtgcggcgccggcaggaaggaaatgggcggggagggccttcgtgcgt |
| | cgccgcgccgccgtccccttctccctctccagcctcggggctgtccgcggggggacggctgcc |
| | ttcgggggggacggggcagggcggggttcggcttctggcgtgtgaccggcggctctagagcct |
| | ctgctaaccatgttcatgccttcttctttttcctacagctcctgggcaacgtgctggttattg |
| | tgctgtctcatcattttggcaaag |
| CK8 | ccactacgggtttaggctgcccatgtaaggaggcaaggcctggggacacccgagatgcctggt |
| SEQ ID NO: 413 | tataattaacccagacatgtggctgccccccccccccccaacacctgctgcctctaaaaataa |
| | ccctgtccctggtggatcccactacgggtttaggctgcccatgtaaggaggcaaggcctgggg |
| | acacccgagatgcctggttataattaacccagacatgtggctgccccccccccccccaacacc |
| | tgctgcctctaaaaataaccctgtccctggtggatcccactacgggtttaggctgcccatgta |
| | aggaggcaaggcctggggacacccgagatgcctggttataattaacccagacatgtggctgcc |
| | ccccccccccccaacacctgctgcctctaaaaataaccctgtccctggtggatcccctgcatg |
| | cgaagatcttcgaacaaggctgtgggggactgagggcaggctgtaacaggcttgggggccagg |
| | gcttatacgtgcctgggactcccaaagtattactgttccatgttcccggcgaagggccagctg |
| | tcccccgccagctagactcagcacttagtttaggaaccagtgagcaagtcagcccttggggca |
| | gcccatacaaggccatggggctgggcaagctgcacgcctgggtccggggtgggcacggtgccc |
| | gggcaacgagctgaaagctcatctgctctcaggggcccctccctggggacagcccctcctggc |
| | tagtcacaccctgtaggctcctctatataacccaggggcacaggggctgccctcattctacca |
| | ccacctccacagcacagacagacactcaggagccagccagcgtcga |
| mU1a | atggaggcggtactatgtagatgagaattcaggagcaaactgggaaaagcaactgcttccaaa |
| SEQ ID NO: 414 | tatttgtgatttttacagtgtagttttggaaaaactcttagcctaccaattcttctaagtgtt |
| | ttaaaatgtgggagccagtacacatgaagttatagagtgttttaatgaggcttaaatatttac |
| | cgtaactatgaaatgctacgcatatcatgctgttcaggctccgtggccacgcaactcatact |
| EF-1α | gggcagagcgcacatcgcccacagtccccgagaagttggggggaggggtcggcaattgaacgg |
| SEQ ID NO: 415 | gtgcctagagaaggtggcgcggggtaaactgggaaagtgatgtcgtgtactggctccgccttt |
| | ttcccgagggtgggggagaaccgtatataagtgcagtagtcgccgtgaacgttctttttcgca |
| | acgggtttgccgccagaacacag |
| TBG | gggctggaagctacctttgacatcatttcctctgcgaatgcatgtataatttctacagaacct |
| SEQ ID NO: 423 | attagaaaggatcacccagcctctgcttttgtacaactttcccttaaaaaactgccaattcca |
| | ctgctgtttggcccaatagtgagaactttttcctgctgcctcttggtgcttttgcctatggcc |
| | cctattctgcctgctgaagacactcttgccagcatggacttaaacccctccagctctgacaat |
| | cctctttctcttttgttttacatgaagggtctggcagccaaagcaatcactcaaagttcaaac |
| | cttatcattttttgctttgttcctcttggccttggttttgtacatcagctttgaaaataccat |
| | cccagggttaatgctggggttaatttataactaagagtgctctagttttgcaatacaggacat |
| | gctataaaaatggaaagat |
| Chimeric Intron | gtaagtatcaaggttacaagacaggtttaaggagaccaatagaaactgggcttgtcgagacag |
| SEQ ID NO: 416 | agaagactcttgcgtttctgataggcacctattggtcttactgacatccactttgcctttctc |
| | tccacag |
| VH4 Intron | gtgagtatctcagggatccagacatggggatatgggaggtgcctctgatcccagggctcactg |
| SEQ ID NO: 417 | tgggtctctctgttcacag |
| 5′ITR | ctgcgcgctcgctcgctcactgaggccgcccgggcaaagcccgggcgtcgggcgacctttggt |
| SEQ ID NO: 418 | cgcccggcctcagtgagcgagcgagcgcgcagagagggagtggccaactccatcactaggggt |
| | tcct |
| 5′-ITR | ctgcgcgctcgctcgctcactgaggccgcccgggcaaagcccgggcgtcgggcgacctttggt |
| (Deleted D- | cgcccggcctcagtgagcgagcgagcgcgcagagagggagtgg |
| sequence for self- | |
| complimentary | |
| AAV) | |
| SEQ ID NO: 419 | |
| 3′-ITR AAV | gaacccctagtgatggagttggccactccctctctgcgcgctcgctcgctcactgaggccgcc |
| SEQ ID NO: 420 | cgggcaaagcccgggcgtcgggcgacctttggtcgcccggcctcagtgagcgagcgagcgcgc |
| | agagagggagtggccaa |
| 3′-ITR | ttggccactccctctctgcgcgctcgctcgctcactgaggccgggcgaccaaaggtcgcccga |
| (Deleted D- | cgcccgggctttgcccgggcggcctcagtgagcgagcgagcgcgcag |
| sequence for self- | |
| complimentary | |
| AAV) | |
| SEQ ID NO: 421 | |

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron (e.g. VH4 intron (SEQ ID NO: 417) or a chimeric intron (SEQ ID NO: 416). In certain embodiments, the viral vectors provided herein comprise a polyadenylation sequence.

Provided are gene expression cassettes and rAAVs comprising gene expression cassettes in which expression of the transgene is controlled by engineered nucleic acid regulatory elements that have more than one regulatory element (promoter or enhancer), including regulatory elements that are arranged in tandem (two or three copies) that promote liver-specific expression, or both liver-specific expression and muscle-specific expression, or both liver-specific and expression and bone-specific expression. These regulatory elements include for the liver-specific expression, LSPX1 (SEQ ID NO: 315), LSPX2 (SEQ ID NO: 316), LTP1 (SEQ ID NO: 317), LTP2 (SEQ ID NO: 318), or LTP3 (SEQ ID NO: 319), liver and muscle expression, LMTP6 (SEQ ID NO: 320), LMTP13 (SEQ ID NO: 321), LMTP14 (SEQ ID NO: 322), LMTP15 (SEQ ID NO: 323), LMTP18 (SEQ ID NO: 324), LMTP19 (SEQ ID NO: 325), or LMTP20 (SEQ ID NO: 326), or liver and bone expression, LBTP1 (SEQ ID NO: 327) or LBTP2 (SEQ ID NO: 328), the sequences of which are provided in Table 1 supra.

### 5.1.4 Signal Peptides

In certain embodiments, the vectors provided herein comprise components that modulate protein delivery. In certain embodiments, the viral vectors provided herein comprise one or more signal peptides. Signal peptides may also be referred to herein as “leader sequences” or “leader peptides”. In certain embodiments, the signal peptides allow for the transgene product to achieve the proper packaging (e.g., glycosylation) in the cell. In certain embodiments, the signal peptides allow for the transgene product to achieve the proper localization in the cell. In certain embodiments, the signal peptides allow for the transgene product to achieve secretion from the cell.

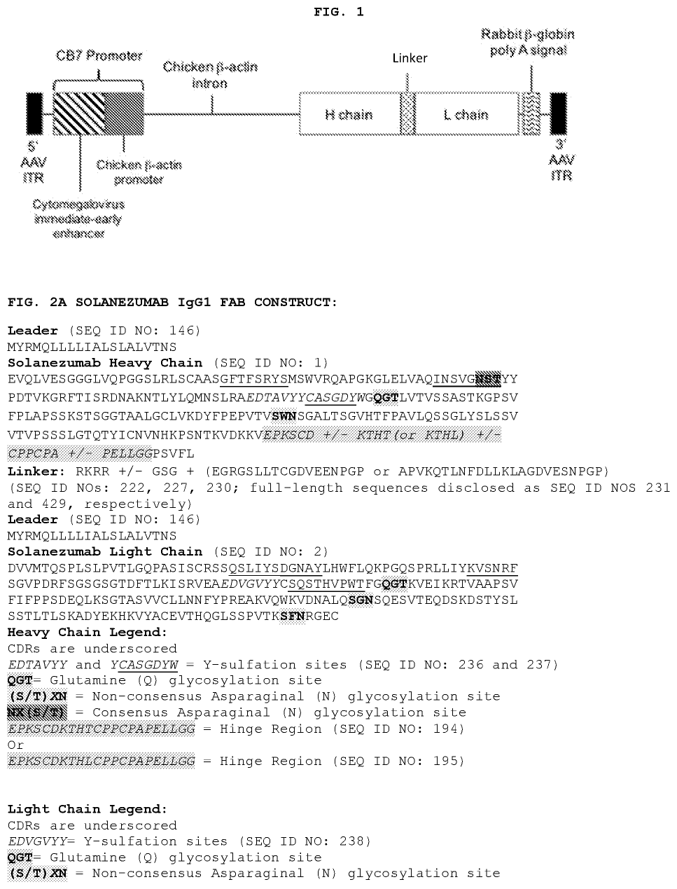

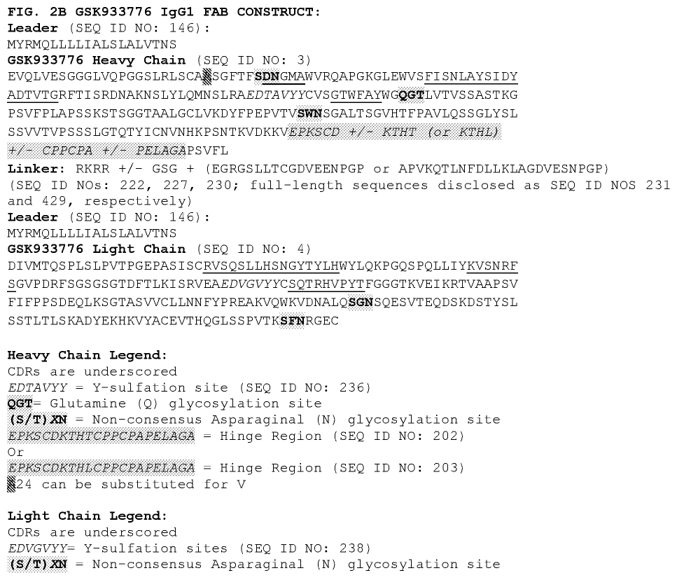

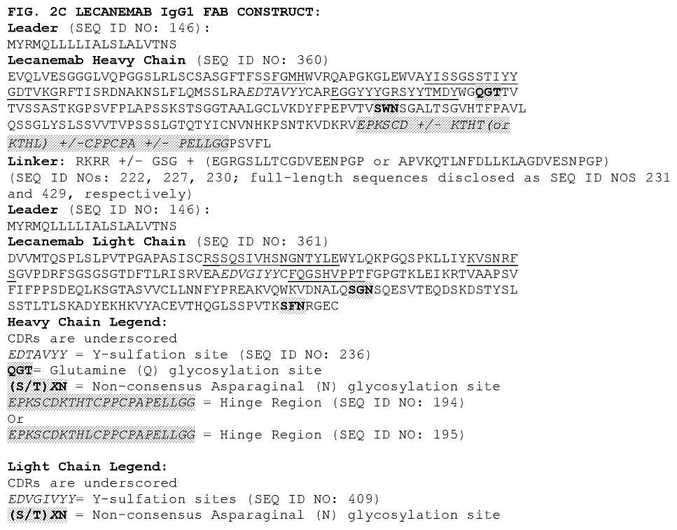

There are two general approaches to select a signal sequence for protein production in a gene therapy context or in cell culture. One approach is to use a signal peptide from proteins homologous to the protein being expressed. For example, a human antibody signal peptide may be used to express IgGs in CHO or other cells. Another approach is to identify signal peptides optimized for the particular host cells used for expression. Signal peptides may be interchanged between different proteins or even between proteins of different organisms, but usually the signal sequences of the most abundant secreted proteins of that cell type are used for protein expression. For example, the signal peptide of human albumin, the most abundant protein in plasma, was found to substantially increase protein production yield in CHO cells. However, certain signal peptides may retain function and exert activity after being cleaved from the expressed protein as “post-targeting functions”. Thus, in specific embodiments, the signal peptide is selected from signal peptides of the most abundant proteins secreted by the cells used for expression to avoid the post-targeting functions. In a certain embodiment, the signal sequence is fused to both the heavy and light chain sequences. An exemplary sequence is MYRMQLLLLIALSLALVTNS (SEQ ID NO: 146) which can be encoded by a nucleotide sequence of SEQ ID NO: 422 (see Table 2, FIGS. 2-19 and FIGS. 29A-29F). Alternatively, signal sequences that are appropriate for expression, and may cause selective expression or directed expression of the HuPTM mAb or Fab or scFv in eye (including CNS), muscle, or liver are provided in Tables 2, 3 and 4, respectively, below.

TABLE 2
Signal peptides for expression in eye/CNS tissue

| | SEQ ID | |
|---|---|---|
| Signal Peptide Origin | NO: | Sequence |
| Mutant interleukin 2 | 146 | MYRMQLLLLIALSLALVTNS |
| signal peptide | | |
| Mutant interleukin 2 | 422 | atgtataggatgcaactgctcctcctgatt |
| signal peptide coding | | gctctgagcctggctcttgtgaccaactct |
| sequence | | |
| VEGF-A signal | 147 | MNFLLSWVHWSLALLLYLHHAKWSQA |
| peptide | | |
| Fibulin-1 signal | 148 | MERAAPSRRVPLPLLLLGGLALLAAGVDA |
| peptide | | |
| Vitronectin signal | 149 | MAPLRPLLILALLAWVALA |
| peptide | | |
| Complement Factor H | 150 | MRLLAKIICLMLWAICVA |
| signal peptide | | |
| Opticin signal peptide | 151 | MRLLAFLSLLALVLQETGT |
| Albumin signal | 152 | MKWVTFISLLFLFSSAYS |
| peptide | | |
| Chymotrypsinogen | 153 | MAFLWLLSCWALLGTTFG |
| signal peptide | | |
| Interleukin-2 signal | 154 | MYRMQLLSCIALILALVTNS |
| peptide | | |
| Trypsinogen-2 signal | 155 | MNLLLILTFVAAAVA |
| peptide | | |

TABLE 3
Signal peptides for expression in muscle cells.

| | SEQ ID | |
|---|---|---|
| Signal Peptide Origin | NO: | Sequence |
| Human SPARC | 156 | MRAWIFFLLCLAGRALA |
| Human Collagen alpha-1(I) | 157 | MFSFVDLRLLLLLAATALLTHG |
| chain | | |
| Human Lactotransferrin | 158 | MKLVFLVLLFLGALGLCLA |
| Human Complement C3 | 159 | MGPTSGPSLIILLLTHLPLALG |
| Human Lumican | 160 | MSLSAFTLFLALIGGTSG |
| Human Gelsolin isoform 1 | 161 | MAPHRPAPALLCALSLALCALSLPVRA |
| Human Pro-cathepsin H | 162 | MWATLPLLCAGAWLLGVPVCGA |
| Human SERPINF1 | 163 | MQALVLLLCIGALLGHSSC |
| Human SERPINE1 | 164 | MQMSPALTCLVLGLALVFGEGSA |
| Human Cathepsin D | 165 | MQPSSLLPLALCLLAAPASA |
| Human TIMP1 | 166 | MAPFEPLASGILLLLWLIAPSRA |
| Human Fibronectin | 167 | MLRGPGPGLLLLAVQCLGTAVPSTGASKSKR |
| Human Complement C1s | 168 | MWCIVLFSLLAWVYA |
| subcomponent | | |
| Human Cathepsin L1 | 169 | MNPTLILAAFCLGIASA |
| Human Cathepsin B | 170 | MWQLWASLCCLLVLANA |
| Human Salivary acidic | 171 | MLLILLSVALLAFSSA |
| proline-rich | | |
| phosphoprotein ½ | | |
| Human Follistatin-related | 172 | MWKRWLALALALVAVAWVRA |
| protein 1 | | |

TABLE 4
Signal peptides for expression in liver cells.

| | SEQ ID | |
|---|---|---|
| Signal Peptide Origin | NO: | Sequence |
| Human Serum albumin | 173 | MKWVTFISLLFLFSSAYS |
| Human α-1 Antitrypsin | 174 | MPSSVSWGILLLAGLCCLVPVSLA |
| (SERPINA1) | | |
| Human Apolipoprotein A-1 | 175 | MKAAVLTLAVLFLTGSQA |
| Human Apolipoprotein A-2 | 176 | MKLLAATVLLLTICSLEG |
| Human Apolipoprotein B-100 | 177 | MDPPRPALLALLALPALLLLLLAGARA |
| Human Coagulation Factor | 178 | MQRVNMIMAESPGLITICLLGYLLSAEC |
| IX | | |
| Human Complement C2 | 179 | MGPLMVLFCLLFLYPGLADS |
| Human Complement | 180 | MWLLVSVILISRISSVGG |
| Factor H-related Protein 2 | | |
| (CFHR2) | | |
| Human Complement | 181 | MLLLFSVILISWVSTVGG |
| Factor H-related Protein 5 | | |
| (CFHR5) | | |
| Human Fibrinogen α-chain | 182 | MFSMRIVCLVLSVVGTAWT |
| (FGA) | | |
| Human Fibrinogen β-chain | 183 | MKRIVIVSWSFHKLKTMKHLLLLLLCVFLVKS |
| (FGB) | | |
| Human Fibrinogen γ-chain | 184 | MSWSLHPRNLILYFYALLFLSSTCVA |
| (FGG) | | |
| Human α-2-HS- | 185 | MKSLVLLLCLAQLWGCHS |
| Glycoprotein (AHSG) | | |
| Human Hemopexin (HPX) | 186 | MARVLGAPVALGLWSLCWSLAIA |
| Human Kininogen-1 | 187 | MKLITILFLCSRLLLSLT |
| Human Mannose-binding | 188 | MSLFPSLPLLLLSMVAASYS |
| protein C (MBL2) | | |
| Human Plasminogen | 189 | MEHKEVVLLLLLFLKSGQG |
| (PLMN) | | |
| Human Prothrombin | 190 | MAHVRGLQLPGCLALAALCSLVHS |
| (Coagulation Factor II) | | |
| Human Secreted | 191 | MISRMEKMTMMMKILIMFALGMNYWSCSG |
| Phosphoprotein 24 | | |
| Human Anti-thrombin-III | 192 | MYSNVIGTVTSGKRKVYLLSLLLIGFWDCVTC |
| (SERPINC1) | | |
| Human Serotransferrin | 193 | MRLAVGALLVCAVLGLCLA |
| (TF) | | |

### 5.1.5 Polycistronic Messages—IRES and 2A Linkers and scFv Constructs

Internal ribosome entry sites. A single construct can be engineered to encode both the heavy and light chains separated by a cleavable linker or IRES so that separate heavy and light chain polypeptides are expressed by the transduced cells. In certain embodiments, the viral vectors provided herein provide polycistronic (e.g., bicistronic) messages. For example, the viral construct can encode the heavy and light chains separated by an internal ribosome entry site (IRES) elements (for examples of the use of IRES elements to create bicistronic vectors see, e.g., Gurtu et al., 1996, Biochem. Biophys. Res. Comm. 229(1):295-8, which is herein incorporated by reference in its entirety). IRES elements bypass the ribosome scanning model and begin translation at internal sites. The use of IRES in AAV is described, for example, in Furling et al., 2001, Gene Ther 8(11): 854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based, AAV9-based or AAVrh10-based vector).

Furin-2A linkers. In other embodiments, the viral vectors provided herein encode the heavy and light chains separated by a cleavable linker such as the self-cleaving 2A and 2A-like peptides, with or without upstream furin cleavage sites, e.g. Furin/2A linkers, such as furin/F2A (F/F2A) or furin/T2A (F/T2A) linkers (Fang et al., 2005, Nature Biotechnology 23: 584-590, Fang, 2007, Mol Ther 15: 1153-9, and Chang, J. et al, MAbs 2015, 7(2):403-412, each of which is incorporated by reference herein in its entirety). For example, a furin/2A linker may be incorporated into an expression cassette to separate the heavy and light chain coding sequences, resulting in a construct with the structure:

Leader-Heavy chain-Furin site-2A site-Leader-Light chain-PolyA.

A 2A site or 2A-like site, such as an F2A site comprising the amino acid sequence RKRR(GSG)APVKQTLNFDLLKLAGDVESNPGP(SEQ ID NO: 231) or a T2A site comprising the amino acid sequence RKRR(GSG)EGRGSLLTCGDVEENPGP (SEQ ID NO: 429), is self-processing, resulting in “cleavage” between the final G and P amino acid residues. Several linkers, with or without an upstream flexible Gly-Ser-Gly (GSG) linker sequence (SEQ ID NO: 427), that could be used include but are not limited to:

T2A:
(SEQ ID NO: 227)
(GSG)EGRGSLLTCGDVEENPGP;
P2A:
(SEQ ID NO: 228)
(GSG)ATNFSLLKQAGDVEENPGP;
E2A:
(SEQ ID NO: 229)
(GSG)QCTNYALLKLAGDVESNPGP;
F2A:
(SEQ ID NO: 230)
(GSG)APVKQTLNFDLLKLAGDVESNPGP

(see also, e.g., Szymczak, et al., 2004, Nature Biotechnol 22(5):589-594, and Donnelly, et al., 2001, J Gen Virol, 82:1013-1025, each of which is incorporated herein by reference). Exemplary nucleotide sequences encoding different parts of the flexible linker are described in Table. 1-1.

TABLE 1-1

| | SEQ | |
|---|---|---|
| ID | ID NO: | Sequence |
| Furin-T2A | 424 | agaaagagaagaggctctggagaag |
| | | gcagaggctccctgctgacatgtgg |
| | | ggatgttgaagagaatcctgggcct |
| Furin | 425 | agaaagagaaga |
| Furin-GSG | 426 | agaaagagaagaggctctgga |
| linker | | |
| GSG | 427 | ggctctgga |
| T2A | 428 | gaaggcagaggctccctgctgacat |
| | | gtggggatgttgaagagaatcctgggcct |

In certain embodiments an additional proteolytic cleavage site, e.g. a furin cleavage site, is incorporated into the expression construct adjacent to the self-processing cleavage site (e.g. 2A or 2A like sequence), thereby providing a means to remove additional amino acids that remain following cleavage by the self processing cleavage sequence. Without being bound to any one theory, a peptide bond is skipped when the ribosome encounters the 2A sequence in the open reading frame, resulting in the termination of translation, or continued translation of the downstream sequence (the light chain). This self-processing sequence results in a string of additional amino acids at the end of the C-terminus of the heavy chain. However, such additional amino acids can then be cleaved by host cell Furin at the furin cleavage site(s), e.g. located immediately prior to the 2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15(6):1153-1159; Luke, 2012, Innovations in Biotechnology, Ch. 8, 161-186). Furin linkers that may be used comprise a series of four basic amino acids, for example, RKRR (SEQ ID NO: 222), RRRR (SEQ ID NO: 223), RRKR (SEQ ID NO: 224), or RKKR (SEQ ID NO: 225). Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR (SEQ ID NO: 222), RRRR (SEQ ID NO: 223), RRKR (SEQ ID NO: 224), or RKKR (SEQ ID NO: 225). In certain embodiments, once the linker is cleaved by a carboxypeptidase, no additional amino acids remain. In certain embodiments, 0.5% to 1%, 1% to 2%, 5%, 10%, 15%, or 20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R-X-K/R-R, such that the additional amino acids on the C-terminus of the heavy chain are R, RX, RXK, RXR, RXKR, or RXRR, where X is any amino acid, for example, alanine (A). In certain embodiments, no additional amino acids may remain on the C-terminus of the heavy chain.

Flexible peptide linker. In some embodiments, a single construct can be engineered to encode both the heavy and light chains (e.g. the heavy and light chain variable domains) separated by a flexible peptide linker such as those encoding a scFv. A flexible peptide linker can be composed of flexible residues like glycine and serine so that the adjacent heavy chain and light chain domains are free to move relative to one another. The construct may be arranged such that the heavy chain variable domain is at the N-terminus of the scFv, followed by the linker and then the light chain variable domain. Alternatively, the construct may be arranged such that the light chain variable domain is at the N-terminus of the scFv, followed by the linker and then the heavy chain variable domain. That is, the components may be arranged as NH₂—V_{L}-linker-V_{H}—COOH or NH₂—V_{H}-linker-V_{L}—COOH.

In certain embodiments, an expression cassette described herein is contained within a viral vector with a restraint on the size of the polynucleotide(s) therein. In certain embodiments, the expression cassette is contained within an AAV virus-based vector. Due to the size restraints of certain vectors, the vector may or may not accommodate the coding sequences for the full heavy and light chains of the therapeutic antibody but may accommodate the coding sequences of the heavy and light chains of antigen binding fragments, such as the heavy and light chains of a Fab or F(ab′)₂ fragment or an scFv. In particular, the AAV vectors described herein may accommodate a transgene of approximately 4.7 kilobases. For constructs such as that in FIG. 1 that contains the CB7 promoter, the chicken β-actin intron, rabbit β-globin polyA signal, and ITRs, the therapeutic antibody encoded may be approximately 752 amino acids. Substitution of smaller expression elements would permit the expression of larger protein products, such as full-length therapeutic antibodies.

### 5.1.6 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half-life of the transgene. In certain embodiments, the UTRs are optimized for the stability of the mRNA of the transgene. In certain embodiments, the UTRs are optimized for the secondary structure of the mRNA of the transgene.

### 5.1.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1):364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety). In preferred embodiments, nucleotide sequences encoding the ITRs may, for example, comprise the nucleotide sequences of SEQ ID NOS: 418 (5′-ITR) or 420 (3′-ITR). In certain embodiments, the modified ITRs used to produce self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety). In preferred embodiments, nucleotide sequences encoding the modified ITRs may, for example, comprise the nucleotide sequences of SEQ ID NOS: 419 (5′-ITR) or 421 (3′-ITR).

### 5.1.8 Transgenes

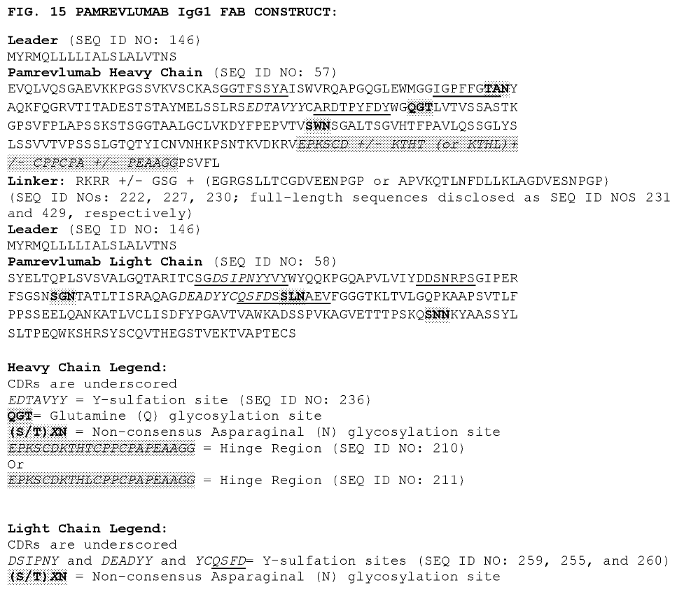

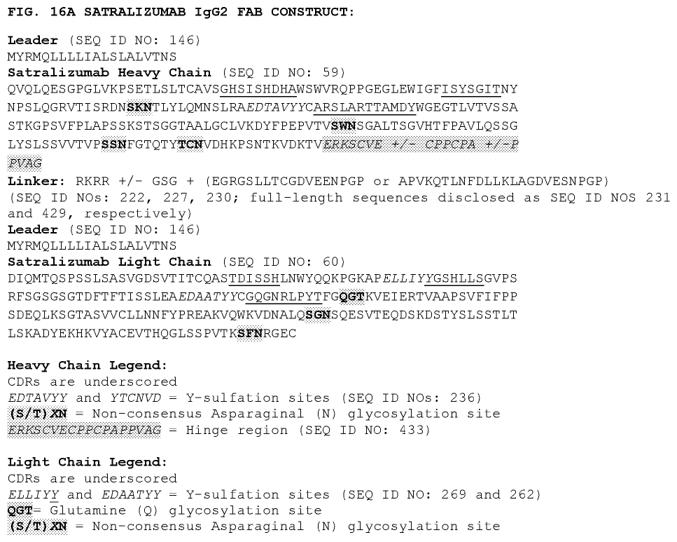

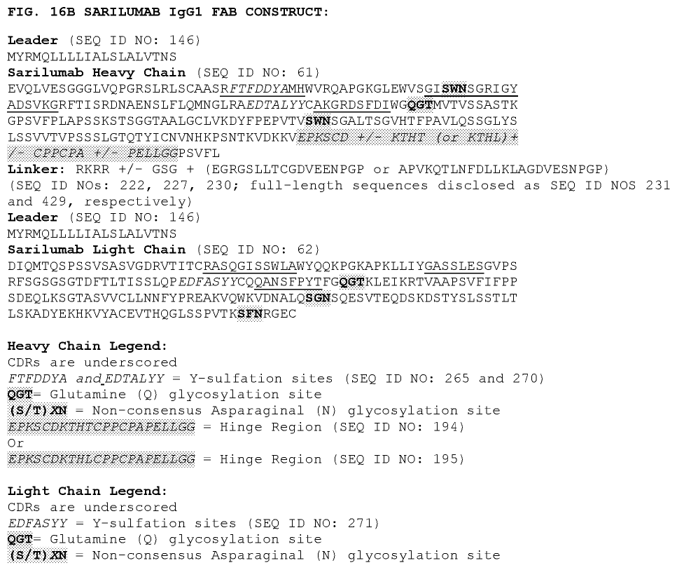

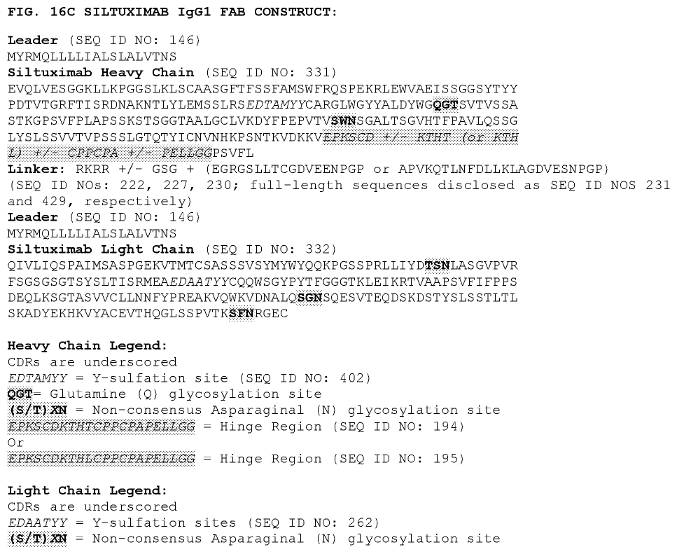

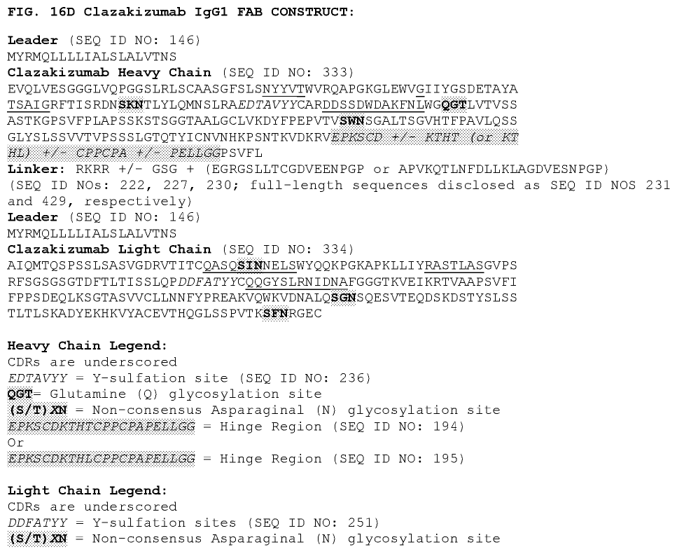

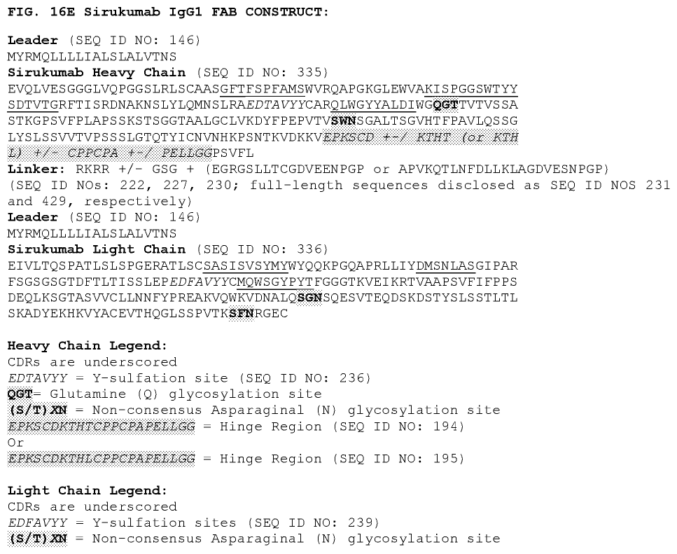

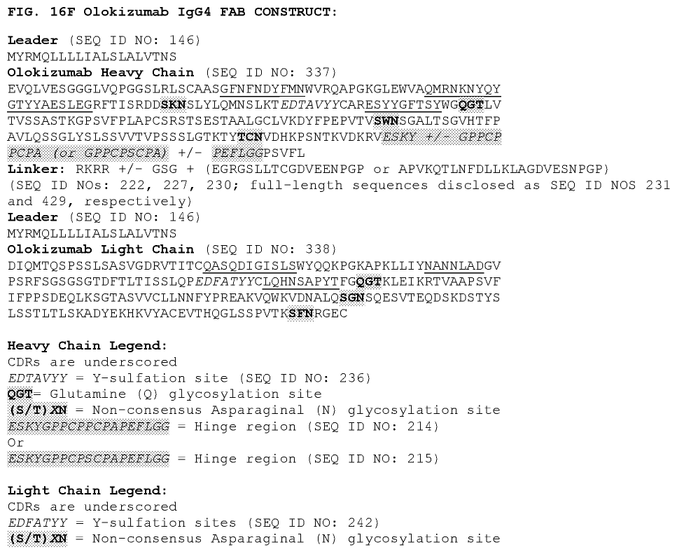

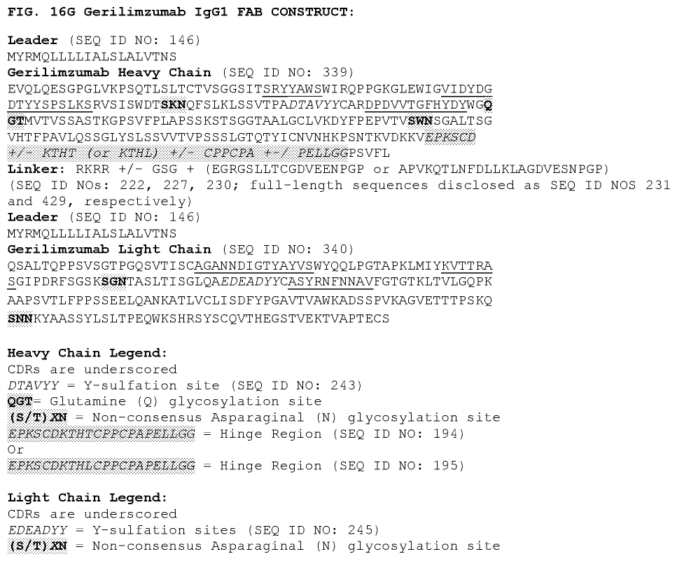

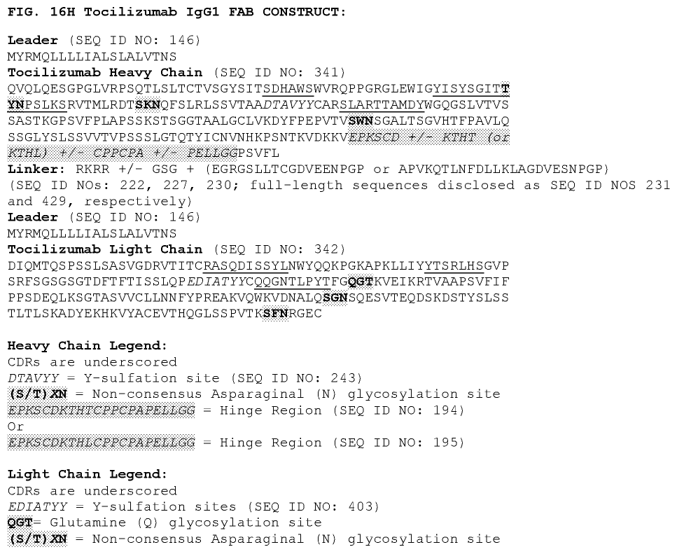

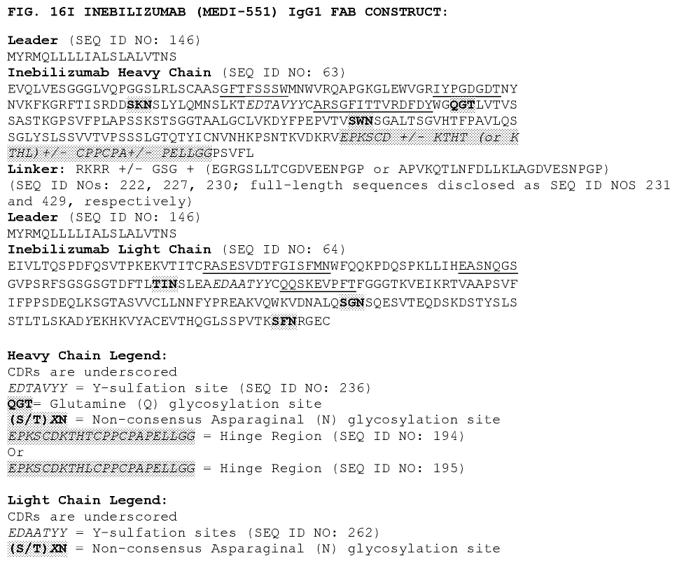

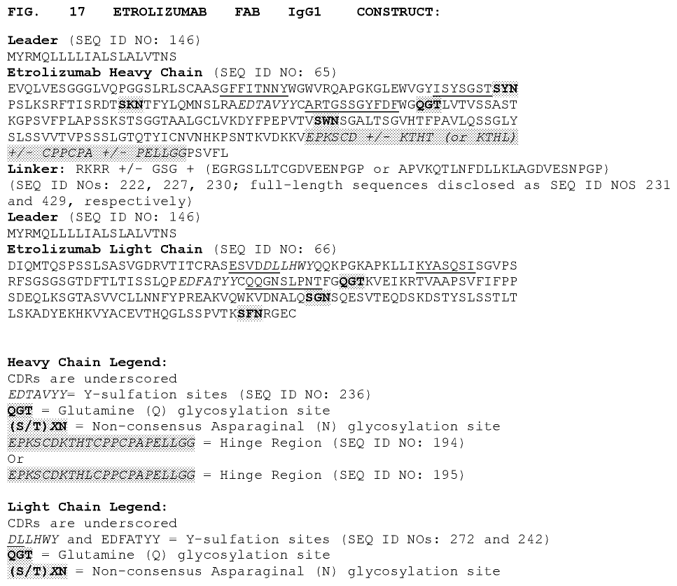

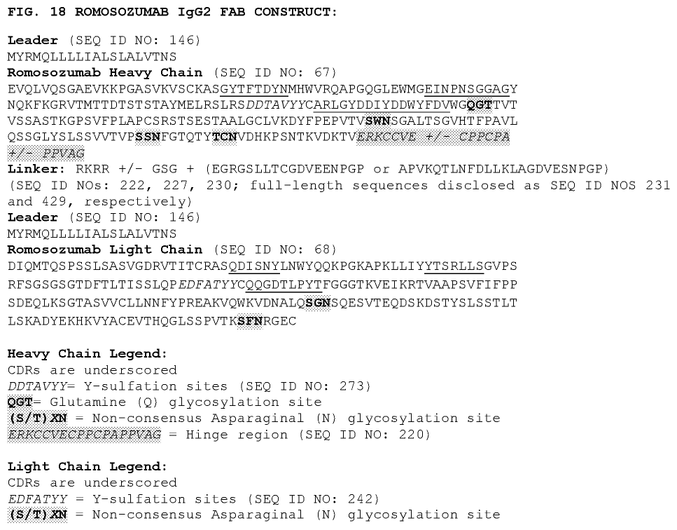

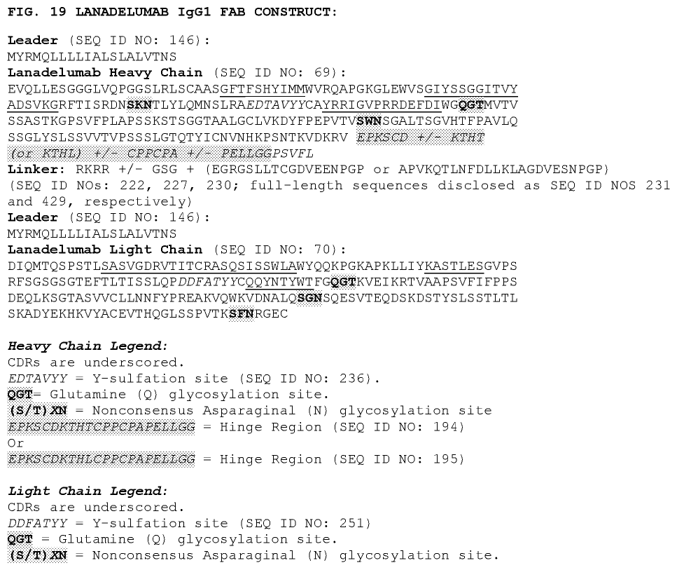

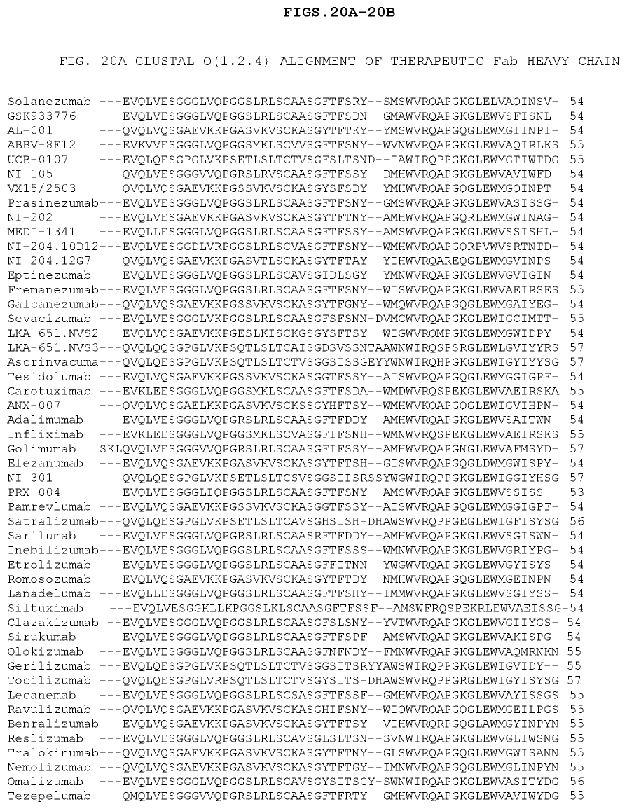

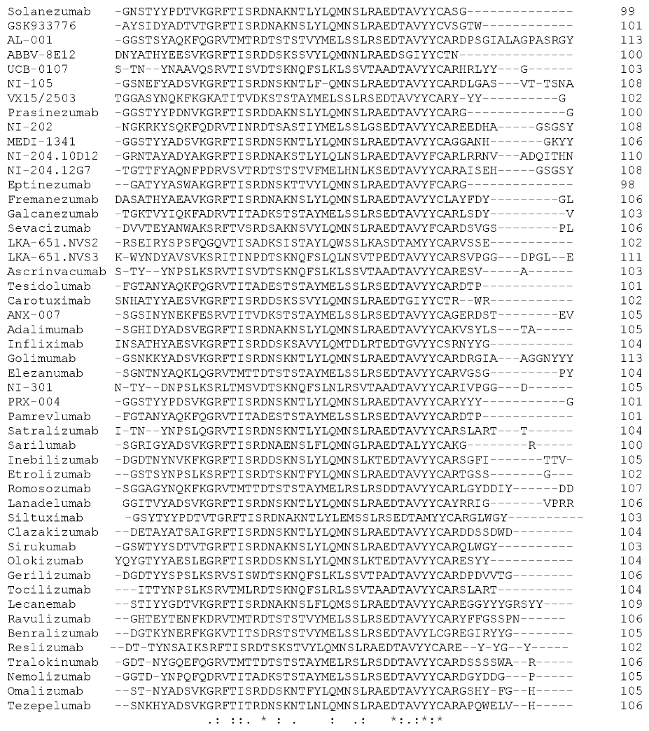

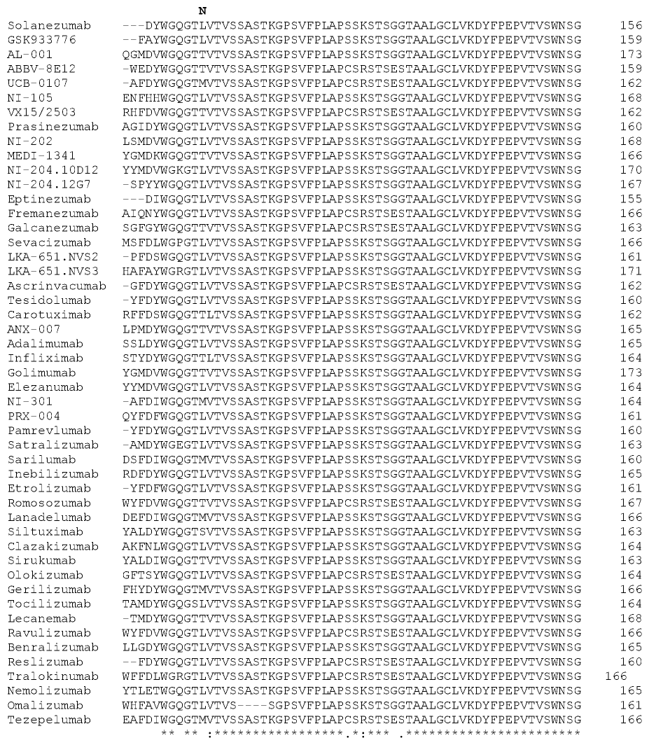

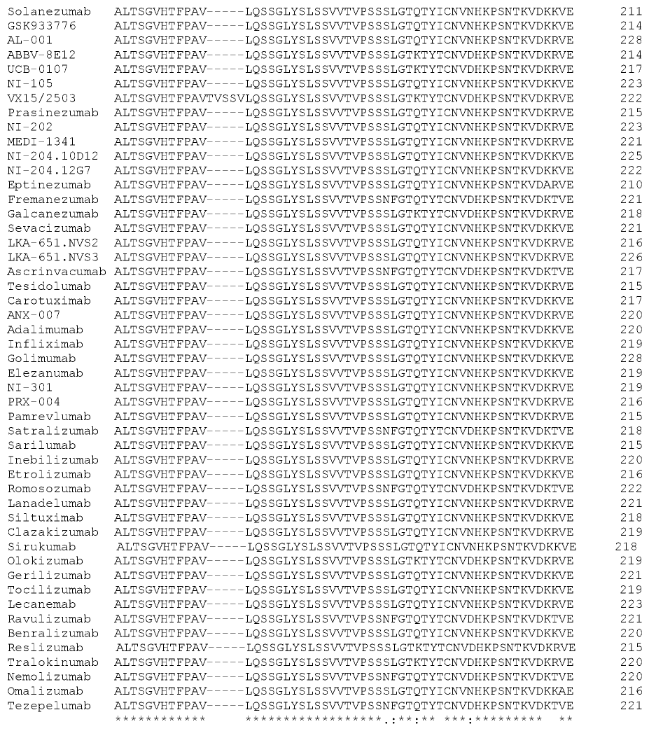

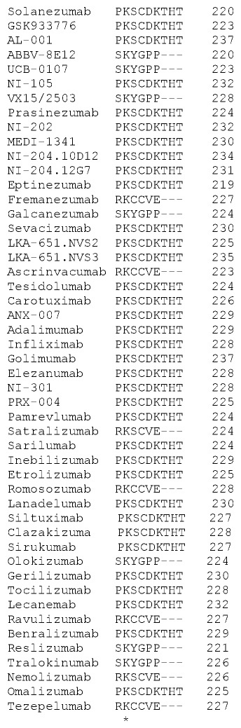

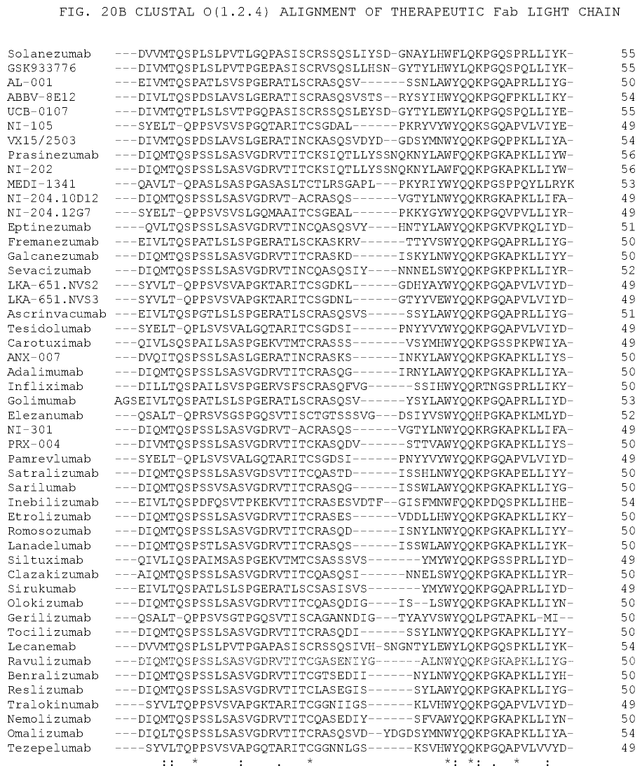

The transgenes encode a HuPTM mAb, either as a full-length antibody or an antigen binding fragment thereof, e.g. a Fab fragment (an HuGlyFab) or a F(ab′)₂ or an scFv based upon a therapeutic antibody disclosed herein. In specific embodiments, the HuPTM mAb or antigen binding fragment, particularly the HuGlyFab, are engineered to contain additional glycosylation sites on the Fab domain (e.g., see Courtois et al., 2016, mAbs 8: 99-112 which is incorporated by reference herein in its entirety for it description of sites of hyperglycosylation on a Fab domain). FIG. 20 provides alignments of the Fab heavy and light chains of the therapeutic antibodies disclosed herein and highlights in green residues that may be substituted with an asparagine or, in some instances, a serine, resulting in hyperglycosylation. In addition, for the HuPTM mAb comprising an Fc domain, the Fc domain may be engineered to alter the glycosylation site at N297 to prevent glycosylation at that site (for example, a substitution at N297 for another amino acid and/or a substitution at T297 for a residue that is not a T or S to knock out the glycosylation site). Such Fc domains are “aglycosylated”.

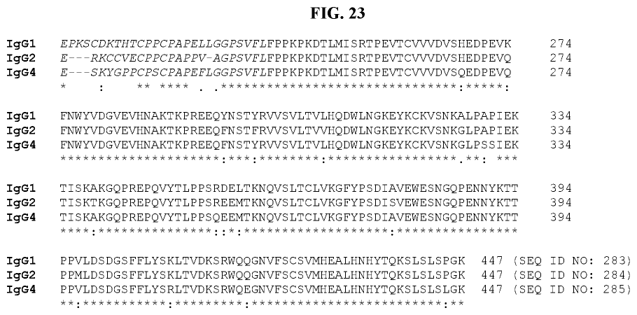

In certain embodiments, the transgenes encode either a full-length antibody or an antigen binding fragment thereof with the coding sequence of the heavy and light chains. Such transgenes encoding the full-length antibody comprise the Fab portion and an Fc region. The Fc region is further discussed in Section 5.1.9. Exemplary sequences are provided in FIG. 23 and Table 7.

In some embodiments, antigen binding fragments are advantageously used. FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F provide the amino acid sequences of the heavy and light chains of the Fab fragments of the therapeutic antibodies (see also Table 5, which provides the amino acid sequences of the Fab heavy and light chains of the therapeutic antibodies). In some embodiments, the transgene for expression of a full-length antibody may comprise the nucleotide sequences encoding the heavy and light chain sequences using nucleotide sequences that encode the Fab portion of the heavy chain, including the hinge region sequence, plus the Fc polypeptide of the heavy chain for the appropriate isotype as described further herein and the light chain. Nucleotide sequences encoding the Fab fragment portions of the heavy and light chains of the therapeutic antibodies disclosed herein are provided in Table 6. Certain of these nucleotide sequences are codon optimized for expression in human cells. Sequences for lanadelumab and adalimumab coding transgenes are provided in Tables 8 and 17, respectively. The transgene may encode a Fab fragment using nucleotide sequences encoding the sequences provided in FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F, but not including the portion of the hinge region on the heavy chain that forms interchain di-sulfide bonds (e.g., the portion containing the sequence CPPCPA (SEQ ID NO: 232)). Heavy chain Fab domain sequences that do not contain a CPPCP (SEQ ID NO: 233) sequence of the hinge region at the C-terminus will not form intrachain disulfide bonds and, thus, will form Fab fragments with the corresponding light chain Fab domain sequences, whereas those heavy chain Fab domain sequences with a portion of the hinge region at the C-terminus containing the sequence CPPCP (SEQ ID NO: 233) will form intrachain disulfide bonds and, thus, will form Fab₂ fragments. For example, in some embodiments, the transgene may encode a scFv comprising a light chain variable domain and a heavy chain variable domain connected by a flexible linker in between (where the heavy chain variable domain may be either at the N-terminal end or the C-terminal end of the scFv), and optionally, may further comprise a Fc polypeptide (e.g., IgG1, IgG2, IgG3, or IgG4) on the C-terminal end of the heavy chain. Alternatively, in other embodiments, the transgene may encode F(ab′)₂ fragments comprising a nucleotide sequence that encodes the light chain and the heavy chain sequence that includes at least the sequence CPPCA (SEQ ID NO: 430) of the hinge region, as depicted in FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F which depict various regions of the hinge region that may be included at the C-terminus of the heavy chain sequence. Pre-existing anti-hinge antibodies (AHA) may cause immunogenicity and reduce efficacy. Thus, in certain embodiments, for the IgG1 isotype, C-terminal ends with D221 or ends with a mutation T225L or with L242 can reduce binding to AHA. (See, e.g., Brerski, 2008, J Immunol 181: 3183-92 and Kim, 2016, 8: 1536-1547). For IgG2, the risk of AHA is lower since the hinge region of IgG2 is not as susceptible to enzymatic cleavage required to generate endogenous AHA. (See, e.g., Brerski, 2011, MAbs 3: 558-567).

In certain embodiments, the viral vectors provided herein comprise the following elements in the following order: a) a constitutive or inducible (e.g., hypoxia-inducible or rifamycin-inducible) promoter sequence or a tissue specific promoter/regulatory region, for example, one of the regulatory regions provided in Table 1, and b) a sequence encoding the transgene (e.g., a HuGlyFab). In certain embodiments, the sequence encoding the transgene comprises multiple ORFs separated by IRES elements. In certain embodiments, the ORFs encode the heavy and light chain domains of the HuGlyFab. In certain embodiments, the sequence encoding the transgene comprises multiple subunits in one ORF separated by F/F2A sequences or F/T2A sequences. In certain embodiments, the sequence comprising the transgene encodes the heavy and light chain domains of the HuGlyFab separated by an F/F2A sequence or a F/T2A sequence. In certain embodiments, the sequence comprising the transgene encodes the heavy and light chain variable domains of the HuGlyFab separated by a flexible peptide linker (as an scFv). In certain embodiments, the viral vectors provided herein comprise the following elements in the following order: a) a constitutive or an inducible promoter sequence or a tissue specific promoter, such as one of the promoters or regulatory regions in Table 1, and b) a sequence encoding the transgene (e.g., a HuGlyFab), wherein the transgene comprises a nucleotide sequence encoding a signal peptide, a light chain and a heavy chain Fab portion separated by an IRES element. In certain embodiments, the viral vectors provided herein comprise the following elements in the following order: a) a constitutive or a hypoxia-inducible promoter sequence or regulatory element listed in Table 1, and b) a sequence encoding the transgene comprising a signal peptide, a light chain and a heavy chain sequence separated by a cleavable F/F2A sequence (SEQ ID NO: 231) or a F/T2A sequence (SEQ ID NO: 429) or a flexible peptide linker.

In certain embodiments, the viral vectors provided herein comprise the following elements in the following order: a) a first ITR sequence, b) a first linker sequence, c) a constitutive or an inducible promoter sequence or a tissue specific promoter or regulatory region, d) a second linker sequence, e) an intron sequence, f) a third linker sequence, g) a first UTR sequence, h) a sequence encoding the transgene (e.g., a HuGlyFab), i) a second UTR sequence, j) a fourth linker sequence, k) a poly A sequence, 1) a fifth linker sequence, and m) a second ITR sequence.

In certain embodiments, the viral vectors provided herein comprise the following elements in the following order: a) a first ITR sequence, b) a first linker sequence, c) a constitutive or an inducible promoter sequence or a tissue specific regulatory region, d) a second linker sequence, e) an intron sequence, f) a third linker sequence, g) a first UTR sequence, h) a sequence encoding the transgene (e.g., HuGlyFab), i) a second UTR sequence, j) a fourth linker sequence, k) a poly A sequence, l) a fifth linker sequence, and m) a second ITR sequence, wherein the transgene comprises a signal, and wherein the transgene encodes a light chain and a heavy chain sequence separated by a cleavable F/2A sequence.

### 5.1.9 Fc Region Modifications

In certain embodiments, the transgenes encode full length or substantially full length heavy and light chains that associate to form a full length or intact antibody. (“Substantially intact” or “substantially full length” refers to a mAb having a heavy chain sequence that is at least 95% identical to the full-length heavy chain mAb amino acid sequence and a light chain sequence that is at least 95% identical to the full-length light chain mAb amino acid sequence). Accordingly, the transgenes comprise nucleotide sequences that encode, for example, the light and heavy chains of the Fab fragments of FIGS. 2A-2C, 3, 4A-4C, 5, 6A-6C, 7A-B, 8A-8C, 9A-9C, 10A-10D, 11, 12A-12C, 13, 14A-14B, 15, 16A-16I, 17, 18, 19, and 29A-29F including the hinge region of the heavy chain and C-terminal of the heavy chain of the Fab fragment, an Fc domain peptide. Table 7 provides the amino acid sequences of the Fc polypeptides for certain of the therapeutic antibodies described herein. Alternatively, an IgG1, IgG2, or IgG4 Fc domain, the sequences of which are provided in FIG. 23 may be utilized. As detailed, the transgene may comprise a nucleotide sequence encoding the Fc polypeptide for the therapeutic antibody linked to the nucleotide sequence encoding the heavy chain Fab fragment at the C terminus of the hinge region as provided in Table 6 (with the amino acid sequences provided in FIGS. 2A-2C, 3, 4A-4C, 5, 6A-6C, 7A-B, 8A-8C, 9A-9C, 10A-10D, 11, 12A-12C, 13, 14A-14B, 15, 16A-16I, 17, 18, 19, and 29A-29F and Table 6).

The term “Fc region” refers to a dimer of two “Fc polypeptides” (or “Fc domains”), each “Fc polypeptide” comprising the heavy chain constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to at least the last two constant region immunoglobulin domains of IgA, IgD, and IgG, or the last three constant region immunoglobulin domains of IgE and IgM and may also include part or all of the flexible hinge N-terminal to these domains. For IgG, e.g., “Fc polypeptide” comprises immunoglobulin domains Cgamma2 (Cγ2, often referred to as CH2 domain) and Cgamma3 (Cγ3, also referred to as CH3 domain) and may include the lower part of the hinge domain between Cgamma1 (Cγ1, also referred to as C_{H}1 domain) and CH2 domain. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, Va.). For IgA, e.g., Fc polypeptide comprises immunoglobulin domains Calpha2 (Cα2) and Calpha3 (Cα3) and may include the lower part of the hinge between Calpha1 (Cα1) and Cα2.

In certain embodiments, the Fc polypeptide is that of the therapeutic antibody (see Table 7) or is the Fc polypeptide corresponding to the isotype of the therapeutic antibody (isotype is indicated in FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F). In still other embodiments, the Fc polypeptide is an IgG Fc polypeptide. The Fc polypeptide may be from the IgG1, IgG2, or IgG4 isotype (see FIG. 23 for alignment of IgG1, IgG2 and IgG4 Fc domain sequences, numbered according to EU numbering) or may be an IgG3 Fc domain, depending, for example, upon the desired effector activity of the therapeutic antibody. In some embodiments, the engineered heavy chain constant region (CH), which includes the Fc domain, is chimeric. As such, a chimeric CH region combines CH domains derived from more than one immunoglobulin isotype and/or subtype. For example, the chimeric (or hybrid) CH region comprises part or all of an Fc region from IgG, IgA and/or IgM. In other examples, the chimeric CH region comprises part or all a CH2 domain derived from a human IgG1, human IgG2, or human IgG4 molecule, combined with part or all of a CH3 domain derived from a human IgG1, human IgG2, or human IgG4 molecule. In other embodiments, the chimeric CH region contains a chimeric hinge region.

In some embodiments, the recombinant vectors encode therapeutic antibodies comprising an engineered (mutant) Fc regions, e.g. engineered Fc regions of an IgG constant region. Modifications to an antibody constant region, Fc region or Fc fragment of an IgG antibody may alter one or more effector functions such as Fc receptor binding or neonatal Fc receptor (FcRn) binding and thus half-life, CDC activity, ADCC activity, and/or ADPC activity, compared to a corresponding antibody having a wild-type IgG constant region, or an IgG heavy chain constant region without the recited modification(s). Accordingly, in some embodiments, the antibody may be engineered to provide an antibody constant region, Fc region or Fc fragment of an IgG antibody that exhibits altered binding (as compared to a reference or wild-type constant region without the recited modification(s)) to one or more Fc receptors (e.g., FcγRI, FcγRIIA, FcγRIIB, FcγRIIIA, FcγRIIIB, FcγRIV, or FcRn receptor). In some embodiments, the antibody an antibody constant region, Fc region or Fc fragment of an IgG antibody that exhibits a one or more altered effector functions such as CDC, ADCC, or ADCP activity, compared to a corresponding antibody having a wild-type IgG constant region, or an IgG constant without the recited modification(s).

“Effector function” refers to a biochemical event that results from the interaction of an antibody Fc region with an Fc receptor or ligand. Effector functions include FcγR-mediated effector functions such as ADCC and ADCP and complement-mediated effector functions such as CDC.

An “effector cell” refers to a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and T cells, and may be from any organism including but not limited to humans, mice, rats, rabbits, and monkeys.

“ADCC” or “antibody dependent cell-mediated cytotoxicity” refers to the cell-mediated reaction wherein nonspecific cytotoxic effector (immune) cells that express FcγRs recognize bound antibody on a target cell and subsequently cause lysis of the target cell.

“ADCP” or “antibody dependent cell-mediated phagocytosis” refers to the cell-mediated reaction wherein nonspecific cytotoxic effector (immune) cells that express FcγRs recognize bound antibody on a target cell and subsequently cause phagocytosis of the target cell.

“CDC” or “complement-dependent cytotoxicity” refers to the reaction wherein one or more complement protein components recognize bound antibody on a target cell and subsequently cause lysis of the target cell.

In some embodiments, the modifications of the Fc domain include, but are not limited to, the following modifications and combinations thereof, with reference to EU numbering of an IgG constant region (see FIG. 23): 233, 234, 235, 236, 237, 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

In certain embodiments, the Fc region comprises an amino acid addition, deletion, or substitution of one or more of amino acid residues 251-256, 285-290, 308-314, 385-389, and 428-436 of the IgG. In some embodiments, 251-256, 285-290, 308-314, 385-389, and 428-436 (EU numbering of Kabat; see FIG. 23) is substituted with histidine, arginine, lysine, aspartic acid, glutamic acid, serine, threonine, asparagine, or glutamine. In some embodiments, a non-histidine residue is substituted with a histidine residue. In some embodiments, a histidine residue is substituted with a non-histidine residue.

Enhancement of FcRn binding by an antibody having an engineered Fc leads to preferential binding of the affinity-enhanced antibody to FcRn as compared to antibody having wild-type Fc, and thus leads to a net enhanced recycling of the FcRn-affinity-enhanced antibody, which results in further increased antibody half-life. An enhanced recycling approach allows highly effective targeting and clearance of antigens, including e.g. “high titer” circulating antigens, such as C5, cytokines, or bacterial or viral antigens.

Provided in certain embodiments are modified constant region, Fc region or Fc fragment of an IgG antibody with enhanced binding to FcRn in serum as compared to a wild-type Fc region (without engineered modifications). In some instances, antibodies, e.g. IgG antibodies, are engineered to bind to FcRn at a neutral pH, e.g., at or above pH 7.4, to enhance pH-dependence of binding to FcRn as compared to a wild-type Fc region (without engineered modifications). In some instances, antibodies, e.g. IgG antibodies, are engineered to exhibit enhanced binding (e.g. increased affinity or K_{D}) to FcRn in endosomes (e.g., at an acidic pH, e.g., at or below pH 6.0) relative to a wild-type IgG and/or reference antibody binding to FcRn at an acidic pH, as well as in comparison to binding to FcRn in serum (e.g., at a neutral pH, e.g., at or above pH 7.4). Provided are antibodies with an engineered antibody constant region, Fc region or Fc fragment of an IgG antibody that exhibits an improved serum or resident tissue half-life, compared to a corresponding antibody having a wild-type IgG constant region, or an IgG constant without the recited modification(s);

Non-limiting examples of such Fc modifications include, e.g., a modification at position 250 (e.g., E or Q); 250 and 428 (e.g., L or F); 252 (e.g., LN/Y/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P) (EU numbering; see FIG. 23).

In some embodiments, the Fc region can be a mutant form such as hIgG1 Fc including M252 mutations, e.g. M252Y and S254T and T256E (“YTE mutation”) exhibit enhanced affinity for human FcRn (Dall'Acqua, et al., 2002, J Immunol 169:5171-5180) and subsequent crystal structure of this mutant antibody bound to hFcRn resulting in the creation of two salt bridges (Oganesyan, et al. 2014, JBC 289(11): 7812-7824). Antibodies having the YTE mutation have been administered to monkeys and humans, and have significantly improved pharmacokinetic properties (Haraya, et al., 2019, Drug Metabolism and Pharmacokinetics, 34(1):25-41).

In some embodiments, modifications to one or more amino acid residues in the Fc region may reduce half-life in systemic circulation (serum), however result in improved retainment in tissues (e.g. in the eye) by disabling FcRn binding (e.g. H435A, EU numbering of Kabat) (Ding et al., 2017, MAbs 9:269-284; and Kim, 1999, Eur J Immunol 29:2819).

In some embodiments, the Fc domain may be engineered to activate all, some, or none of the normal Fc effector functions, without affecting the Fc polypeptide's (e.g. antibody's) desired pharmacokinetic properties. Fc polypeptides having altered effector function may be desirable as they may reduce unwanted side effects, such as activation of effector cells, by the therapeutic protein.

Methods to alter or even ablate effector function may include mutation(s) or modification(s) to the hinge region amino acid residues of an antibody. For example, IgG Fc domain mutants comprising 234A, 237A, and 238S substitutions, according to the EU numbering system, exhibit decreased complement dependent lysis and/or cell mediated destruction. Deletions and/or substitutions in the lower hinge, e.g. where positions 233-236 within a hinge domain (EU numbering) are deleted or modified to glycine, have been shown in the art to significantly reduce ADCC and CDC activity.

In specific embodiments, the Fc domain is an aglycosylated Fc domain that has a substitution at residue 297 or 299 to alter the glycosylation site at 297 such that the Fc domain is not glycosylated. Such aglycosylated Fc domains may have reduced ADCC or other effector activity.

Non-limiting examples of proteins comprising mutant and/or chimeric CH regions having altered effector functions, and methods of engineering and testing mutant antibodies, are described in the art, e.g. K. L. Amour, et al., Eur. J. Immunol. 1999, 29:2613-2624; Lazar et al., Proc. Natl. Acad. Sci. USA 2006, 103:4005; US Patent Application Publication No. 20070135620A1 published Jun. 14, 2007; US Patent Application Publication No. 20080154025 A1, published Jun. 26, 2008; US Patent Application Publication No. 20100234572 A1, published Sep. 16, 2010; US Patent Application Publication No. 20120225058 A1, published Sep. 6, 2012; US Patent Application Publication No. 20150337053 A1, published Nov. 26, 2015; International Publication No. WO20/16161010A2 published Oct. 6, 2016; U.S. Pat. No. 9,359,437, issued Jun. 7, 2016; and U.S. Pat. No. 10,053,517, issued Aug. 21, 2018, all of which are herein incorporated by reference.

The C-terminal lysines (-K) conserved in the heavy chain genes of all human IgG subclasses are generally absent from antibodies circulating in serum—the C-terminal lysines are cleaved off in circulation, resulting in a heterogeneous population of circulating IgGs. (van den Bremer et al., 2015, mAbs 7:672-680). In the vectored constructs for full length mAbs, the DNA encoding the C-terminal lysine (-K) or glycine-lysine (-GK) of the Fc terminus can be deleted to produce a more homogeneous antibody product in situ. (See, Hu et al., 2017 Biotechnol. Prog. 33: 786-794 which is incorporated by reference herein in its entirety).

### 5.1.10 Manufacture and Testing of Vectors

The viral vectors provided herein may be manufactured using host cells. The viral vectors provided herein may be manufactured using mammalian host cells, for example, A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (e.g., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

Alternatively, baculovirus expression systems in insect cells may be used to produce AAV vectors. For a review, see Aponte-Ubillus et al., 2018, Appl. Microbiol. Biotechnol. 102:1045-1054 which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Once expressed, characteristics of the expressed product can be determined, including determination of the glycosylation and tyrosine sulfation patterns associated with the HuGlyFab. Glycosylation patterns and methods of determining the same are discussed in Section 5.2.1, while tyrosine sulfation patterns and methods of determining the same are discussed in Section 5.2.2. In addition, benefits resulting from glycosylation/sulfation of the cell-expressed HuGlyFab can be determined using assays known in the art, e.g., the methods described in Sections 5.2.1 and 5.2.2.

### 5.1.10 Compositions

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN′, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

### 5.2 N-Glycosylation, Tyrosine Sulfation, and O-Glycosylation

The amino acid sequence (primary sequence) of HuGlyFabs or HuPTM Fabs, HuPTMmAbs, and HuPTM scFvs disclosed herein each comprises at least one site at which N-glycosylation or tyrosine sulfation takes place (see FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F) for glycosylation and/or sulfation positions within the amino acid sequences of the Fab fragments of the therapeutic antibodies). Post-translational modification also occurs in the Fc domain of full length antibodies, particularly at residue N297 (by EU numbering, see FIG. 23).

Alternatively, mutations may be introduced into the Fc domain to alter the glycosylation site at residue N297 (EU numbering, see FIG. 23), in particular substituting another amino acid for the asparagine at 297 or the threonine at 299 to remove the glycosylation site resulting in an aglycosylated Fc domain.

### 5.2.1 N-Glycosylation

Reverse Glycosylation Sites

The canonical N-glycosylation sequence is known in the art to be Asn-X-Ser (or Thr), wherein X can be any amino acid except Pro. However, it recently has been demonstrated that asparagine (Asn) residues of human antibodies can be glycosylated in the context of a reverse consensus motif, Ser(or Thr)-X-Asn, wherein X can be any amino acid except Pro. See Valliere-Douglass et al., 2009, J. Biol. Chem. 284:32493-32506; and Valliere-Douglass et al., 2010, J. Biol. Chem. 285:16012-16022. As disclosed herein, certain HuGlyFabs and HuPTM scFvs disclosed herein comprise such reverse consensus sequences.

Non-Consensus Glycosylation Sites

In addition to reverse N-glycosylation sites, it recently has been demonstrated that glutamine (Gln) residues of human antibodies can be glycosylated in the context of a non-consensus motif, Gln-Gly-Thr. See Valliere-Douglass et al., 2010, J. Biol. Chem. 285:16012-16022. Surprisingly, certain of the HuGlyFab fragments disclosed herein comprise such non-consensus sequences. In addition, O-glycosylation comprises the addition of N-acetyl-galactosamine to serine or threonine residues by the enzyme. It has been demonstrated that amino acid residues present in the hinge region of antibodies can be O-glycosylated. The possibility of O-glycosylation confers another advantage to the therapeutic antibodies provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098.)

Engineered N-Glycosylation Sites

In certain embodiments, a nucleic acid encoding a HuPTM mAb, HuGlyFab or HuTPM scFv is modified to include 1, 2, 3, 4, 5, 6, 7, 8, 9, 10, or more N-glycosylation sites (including the canonical N-glycosylation consensus sequence, reverse N-glycosylation site, and non-consensus N-glycosylation sites) than would normally be associated with the HuPTM mAb, HuGlyFab or HuPTM scFv (e.g., relative to the number of N-glycosylation sites associated with the HuPTM mAb, HuGlyFab or HuPTM scFv in its unmodified state). In specific embodiments, introduction of glycosylation sites is accomplished by insertion of N-glycosylation sites (including the canonical N-glycosylation consensus sequence, reverse N-glycosylation site, and non-consensus N-glycosylation sites) anywhere in the primary structure of the antigen-binding fragment, so long as said introduction does not impact binding of the antibody or antigen-binding fragment to its antigen. Introduction of glycosylation sites can be accomplished by, e.g., adding new amino acids to the primary structure of the antigen-binding fragment, or the antibody from which the antigen-binding fragment is derived (e.g., the glycosylation sites are added, in full or in part), or by mutating existing amino acids in the antigen-binding fragment, or the antibody from which the antigen-binding fragment is derived, in order to generate the N-glycosylation sites (e.g., amino acids are not added to the antigen-binding fragment/antibody, but selected amino acids of the antigen-binding fragment/antibody are mutated so as to form N-glycosylation sites). Those of skill in the art will recognize that the amino acid sequence of a protein can be readily modified using approaches known in the art, e.g., recombinant approaches that include modification of the nucleic acid sequence encoding the protein.

In a specific embodiment, a HuGlyMab or antigen-binding fragment is modified such that, when expressed in mammalian cells, such as retina, CNS, liver or muscle cells, it can be hyperglycosylated. See Courtois et al., 2016, mAbs 8:99-112 which is incorporated by reference herein in its entirety.

N-Glycosylation of HuPTM mAbs and HuPTM Antigen-Binding Fragments

Unlike small molecule drugs, biologics usually comprise a mixture of many variants with different modifications or forms that could have a different potency, pharmacokinetics, and/or safety profile. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment provided herein can be, for example, to slow or arrest the progression of a disease or abnormal condition or to reduce the severity of one or more symptoms associated with the disease or abnormal condition.

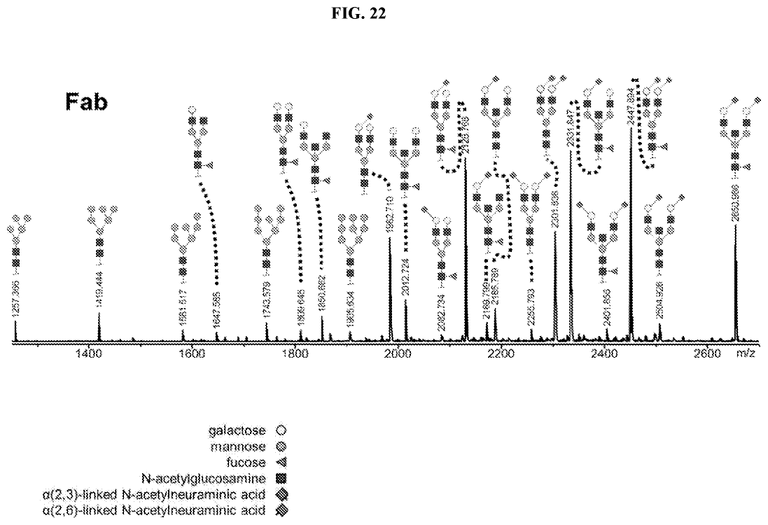

When a HuPTM mAb, HuGlyFab or HuPTM scFv is expressed in a human cell, the N-glycosylation sites of the antigen-binding fragment can be glycosylated with various different glycans. N-glycans of antigen-binding fragments and the Fc domain have been characterized in the art. For example, Bondt et al., 2014, Mol. & Cell. Proteomics 13.11:3029-3039 (incorporated by reference herein in its entirety for its disclosure of Fab-associated N-glycans; see also, FIG. 22) characterizes glycans associated with Fabs, and demonstrates that Fab and Fc portions of antibodies comprise distinct glycosylation patterns, with Fab glycans being high in galactosylation, sialylation, and bisection (e.g., with bisecting GlcNAc) but low in fucosylation with respect to Fc glycans. Like Bondt, Huang et al., 2006, Anal. Biochem. 349:197-207 (incorporated by reference herein in its entirety for it disclosure of Fab-associated N-glycans) found that most glycans of Fabs are sialylated. However, in the Fab of the antibody examined by Huang (which was produced in a murine cell background), the identified sialic residues were N-Glycolylneuraminic acid (“Neu5Gc” or “NeuGc”) (which is not natural to humans) instead of N-acetylneuraminic acid (“Neu5Ac,” the predominant human sialic acid). In addition, Song et al., 2014, Anal. Chem. 86:5661-5666 (incorporated by reference herein in its entirety for its disclosure of Fab-associated N-glycans) describes a library of N-glycans associated with commercially available antibodies.

Glycosylation of the Fc domain has been characterized and is a single N-linked glycan at asparagine 297 (EU numbering; see FIG. 23). The glycan plays an integral structural and functional role, impacting antibody effector function, such as binding to Fc receptor (see, for example, Jennewein and Alter, 2017, Trends In Immunology 38:358 for a discussion of the role of Fc glycosylation in antibody function). Removal of the Fc region glycan almost completely ablates effector function (Jennewien and Alter at 362). The composition of the Fc glycan has been shown to impact effector function, for example hypergalactosylation and reduction in fucosylation have been shown to increase ADCC activity while sialylation correlates with anti-inflammatory effects (Id. at 364). Disease states, genetics and even diet can impact the composition of the Fc glycan in vivo. For recombinantly expressed antibodies, the glycan composition can differ significantly by the type of host cell used for recombinant expression and strategies are available to control and modify the composition of the glycan in therapeutic antibodies recombinantly expressed in cell culture, such as CHO to alter effector function (see, for example, US 2014/0193404 by Hansen et al.). Accordingly, the HuPTM mAbs provided herein may advantageously have a glycan at N297 that is more like the native, human glycan composition than antibodies expressed in non-human host cells.

Importantly, when the HuPTM mAb, HuGlyFab or HuPTM scFv are expressed in human cells, the need for in vitro production in prokaryotic host cells (e.g., E. coli) or eukaryotic host cells (e.g., CHO cells or NS0 cells) is circumvented. Instead, as a result of the methods described herein, N-glycosylation sites of the HuPTM mAb, HuGlyFab or HuPTM scFv are advantageously decorated with glycans relevant to and beneficial to treatment of humans. Such an advantage is unattainable when CHO cells, NS0 cells, or E. coli are utilized in antibody/antigen-binding fragment production, because e.g., CHO cells (1) do not express 2,6 sialyltransferase and thus cannot add 2,6 sialic acid during N-glycosylation; (2) can add Neu5Gc as sialic acid instead of Neu5Ac; and (3) can also produce an immunogenic glycan, the α-Gal antigen, which reacts with anti-α-Gal antibodies present in most individuals, which at high concentrations can trigger anaphylaxis; and because (4) E. coli does not naturally contain components needed for N-glycosylation.

Assays for determining the glycosylation pattern of antibodies, including antigen-binding fragments are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. Glycans may also be released using enzymes such as glycosidases or endoglycosidases, such as PNGase F and Endo H, which cleave cleanly and with fewer side reactions than hydrazines. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low or high molecular weight can be analyzed by MALDI-MS/MS and the result used to confirm the glycan sequence. Each peak in the chromatogram corresponds to a polymer, e.g., glycan, consisting of a certain number of repeat units and fragments, e.g., sugar residues, thereof. The chromatogram thus allows measurement of the polymer, e.g., glycan, length distribution. The elution time is an indication for polymer length, while fluorescence intensity correlates with molar abundance for the respective polymer, e.g., glycan. Other methods for assessing glycans associated with antigen-binding fragments include those described by Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666.

Homogeneity or heterogeneity of the glycan patterns associated with antibodies (including antigen-binding fragments), as it relates to both glycan length or size and numbers glycans present across glycosylation sites, can be assessed using methods known in the art, e.g., methods that measure glycan length or size and hydrodynamic radius. HPLC, such as size exclusion, normal phase, reversed phase, and anion exchange HPLC, as well as capillary electrophoresis, allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can be measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

In certain embodiments, the HuPTM mAbs, or antigen binding fragments thereof, also do not contain detectable NeuGc and/or α-Gal. By “detectable NeuGc” or “detectable α-Gal” or “does not contain or does not have NeuGc or α-Gal” means herein that the HuPTM mAb or antigen-binding fragment, does not contain NeuGc or α-Gal moieties detectable by standard assay methods known in the art. For example, NeuGc may be detected by HPLC according to Hara et al., 1989, “Highly Sensitive Determination of N-Acetyl- and N-Glycolylneuraminic Acids in Human Serum and Urine and Rat Serum by Reversed-Phase Liquid Chromatography with Fluorescence Detection.” J. Chromatogr., B: Biomed. 377, 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring α-Gal epitope expression on cells by a monoclonal anti-Gal antibody.” Transplantation. 65(8):1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5):699-710. See also the references cited in Platts-Mills et al., 2015, “Anaphylaxis to the Carbohydrate Side-Chain Alpha-gal” Immunol Allergy Clin North Am. 35(2): 247-260.

Benefits of N-Glycosylation

N-glycosylation confers numerous benefits on the HuPTM mAb, HuGlyFab or HuPTM scFv described herein. Such benefits are unattainable by production of antigen-binding fragments in E. coli, because E. coli does not naturally possess components needed for N-glycosylation. Further, some benefits are unattainable through antibody production in, e.g., CHO cells (or murine cells such as NS0 cells), because CHO cells lack components needed for addition of certain glycans (e.g., 2,6 sialic acid and bisecting GlcNAc) and because either CHO or murine cell lines add N—N-Glycolylneuraminic acid (“Neu5Gc” or “NeuGc”) which is not natural to humans (and potentially immunogenic), instead of N-Acetylneuraminic acid (“Neu5Ac”) the predominant human sialic acid. See, e.g., Dumont et al., 2015, Crit. Rev. Biotechnol. 36(6):1110-1122; Huang et al., 2006, Anal. Biochem. 349:197-207 (NeuGc is the predominant sialic acid in murine cell lines such as SP2/0 and NS0); and Song et al., 2014, Anal. Chem. 86:5661-5666, each of which is incorporated by reference herein in its entirety). Moreover, CHO cells can also produce an immunogenic glycan, the α-Gal antigen, which reacts with anti-α-Gal antibodies present in most individuals, which at high concentrations can trigger anaphylaxis. See, e.g., Bosques, 2010, Nat. Biotech. 28:1153-1156. The human glycosylation pattern of the HuGlyFab of HuPTM scFv described herein should reduce immunogenicity of the transgene product and improve efficacy.

While non-canonical glycosylation sites usually result in low level glycosylation (e.g., 1-5%) of the antibody population, the functional benefits may be significant (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement.

The presence of sialic acid on HuPTM mAb, HuGlyFab or HuPTM scFv used in the methods described herein can impact clearance rate of the HuPTM mAb, HuGlyFab or HuPTM scFv. Accordingly, sialic acid patterns of a HuPTM mAb, HuGlyFab or HuPTM scFv can be used to generate a therapeutic having an optimized clearance rate. Methods of assessing antigen-binding fragment clearance rate are known in the art. See, e.g., Huang et al., 2006, Anal. Biochem. 349:197-207.

In another specific embodiment, a benefit conferred by N-glycosylation is reduced aggregation. Occupied N-glycosylation sites can mask aggregation prone amino acid residues, resulting in decreased aggregation. Such N-glycosylation sites can be native to an antigen-binding fragment used herein or engineered into an antigen-binding fragment used herein, resulting in HuGlyFab or HuPTM scFv that is less prone to aggregation when expressed, e.g., expressed in human cells. Methods of assessing aggregation of antibodies are known in the art. See, e.g., Courtois et al., 2016, mAbs 8:99-112 which is incorporated by reference herein in its entirety.

In another specific embodiment, a benefit conferred by N-glycosylation is reduced immunogenicity. Such N-glycosylation sites can be native to an antigen-binding fragment used herein or engineered into an antigen-binding fragment used herein, resulting in HuPTM mAb, HuGlyFab or HuPTM scFv that is less prone to immunogenicity when expressed, e.g., expressed in human retinal cells, human CNS cells, human liver cells or human muscle cells.

In another specific embodiment, a benefit conferred by N-glycosylation is protein stability. N-glycosylation of proteins is well-known to confer stability on them, and methods of assessing protein stability resulting from N-glycosylation are known in the art. See, e.g., Sola and Griebenow, 2009, J Pharm Sci., 98(4): 1223-1245.

In another specific embodiment, a benefit conferred by N-glycosylation is altered binding affinity. It is known in the art that the presence of N-glycosylation sites in the variable domains of an antibody can increase the affinity of the antibody for its antigen. See, e.g., Bovenkamp et al., 2016, J. Immunol. 196:1435-1441. Assays for measuring antibody binding affinity are known in the art. See, e.g., Wright et al., 1991, EMBO J. 10:2717-2723; and Leibiger et al., 1999, Biochem. J. 338:529-538.

### 5.2.2 Tyrosine Sulfation

Tyrosine sulfation occurs at tyrosine (Y) residues with glutamate (E) or aspartate (D) within +5 to −5 position of Y, and where position −1 of Y is a neutral or acidic charged amino acid, but not a basic amino acid, e.g., arginine (R), lysine (K), or histidine (H) that abolishes sulfation. The HuGlyFabs and HuPTM scFvs described herein comprise tyrosine sulfation sites (see FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F).

Importantly, tyrosine-sulfated antigen-binding fragments cannot be produced in E. coli, which naturally does not possess the enzymes required for tyrosine-sulfation. Further, CHO cells are deficient for tyrosine sulfation—they are not secretory cells and have a limited capacity for post-translational tyrosine-sulfation. See, e.g., Mikkelsen & Ezban, 1991, Biochemistry 30: 1533-1537. Advantageously, the methods provided herein call for expression of HuPTM Fab in human cells that are secretory and have capacity for tyrosine sulfation.

Tyrosine sulfation is advantageous for several reasons. For example, tyrosine-sulfation of the antigen-binding fragment of therapeutic antibodies against targets has been shown to dramatically increase avidity for antigen and activity. See, e.g., Loos et al., 2015, PNAS 112: 12675-12680, and Choe et al., 2003, Cell 114: 161-170. Assays for detection tyrosine sulfation are known in the art. See, e.g., Yang et al., 2015, Molecules 20:2138-2164.

### 5.2.3 O-Glycosylation

O-glycosylation comprises the addition of N-acetyl-galactosamine to serine or threonine residues by the enzyme. It has been demonstrated that amino acid residues present in the hinge region of antibodies can be O-glycosylated. In certain embodiments, the HuGlyFab comprise all or a portion of their hinge region, and thus are capable of being O-glycosylated when expressed in human cells. The possibility of O-glycosylation confers another advantage to the HuGlyFab provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098.) O-glycosylated HuGlyFab, by virtue of possessing glycans, shares advantageous characteristics with N-glycosylated HuGlyFab (as discussed above).

### 5.3 Vectored Therapeutic Antibodies

### 5.3.1 Anti-ABeta HuPTM Constructs and Formulations for Alzheimer's Disease

Compositions and methods are described for the delivery of HuPTM mAbs and antigen-binding fragments thereof, such as HuPTM Fabs, that bind to amyloid beta (Aβ or Abeta) peptides derived from the amyloid precursor protein that may have benefit in treating Alzheimer's disease (AD) and the like. In particular embodiments, the HuPTM mAb is solanezumab, or GSK933776, or lecanemab, or an antigen binding fragment of one of the foregoing. The amino acid sequences of Fab fragments of these antibodies are provided in FIGS. 2A-C, respectively. Delivery may be accomplished via gene therapy—e.g., by administering a viral vector or other DNA expression construct encoding an Aβ-binding HuPTM mAb (or an antigen binding fragment and/or a hyperglycosylated derivative or other derivative, thereof) to patients (human subjects) diagnosed with, or having one or more symptoms of, AD, to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to Aβ that can be administered to deliver the HuPTM mAb or antigen binding fragment in a patient. The transgene is a nucleic acid comprising the nucleotide sequences encoding an antigen binding fragment of an antibody that binds to Aβ, such as solanezumab, lecanemab, or GSK933776, or variants there of as detailed herein. The transgene may also encode an anti-Aβ antigen binding fragment that contains additional glycosylation sites (e.g., see Courtois et al., 2016, mAbs 8: 99-112 which is incorporated by reference herein in its entirety).

In certain embodiments, the anti-Aβ antigen-binding fragment transgene comprises the nucleotide sequences encoding the heavy and light chains of the Fab portion of solanezumab (having amino acid sequences of SEQ ID NOs. 1 and 2, respectively, see Table 5 and FIG. 2A). The nucleotide sequences may be codon optimized for expression in human cells. The nucleotide sequences may, for example, comprise the nucleotide sequences of SEQ ID NO: 71 (encoding the solanezumab heavy chain Fab portion) and SEQ ID NO: 72 (encoding the solanezumab light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the Fab fragments, including the heavy and light chain variable domain sequences and C_{L} and C_{H}1 sequences, the transgenes may comprise, at the C-terminus of the heavy chain C_{H}1 sequence, all or a portion of the hinge region. In specific embodiments, the anti-Aβ-antigen binding domain has a heavy chain variable domain and C_{H}1 domain of SEQ ID NO: 1 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 2A. These hinge regions may be encoded by nucleotide sequences at the 3′ end of SEQ ID NO: 71 by the hinge region encoding sequences set forth in Table 5 (SEQ ID NO: 71). In another embodiment, the transgenes comprise the amino acid sequences encoding the full length (or substantially full length) heavy and light chains of the antibody, comprising the Fc domain at the C terminus of the heavy chain, e.g. an IgG1 Fc domain, such as SEQ ID NO: 290 of Table 7, SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-Aβ antigen-binding fragment transgene encodes an Aβ antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-Aβ antigen-binding fragment transgene encodes an Aβ antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-Aβ antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In specific embodiments, the Aβ antigen binding fragment comprises a heavy chain comprising an amino acid sequence of SEQ ID NO: 1 with 1, 2, 3, 4, 5, 6, 7, 8, 9, 10, 11, 12, 13, 14, 15 or more amino acid substitutions, insertions or deletions, and the substitutions, insertions or deletions are made for example, in the framework regions (e.g., those regions outside of the CDRs, which CDRs are underlined in FIG. 2A) or are substitutions with an amino acid present at that position in the heavy chain of one or more of the other therapeutic antibodies, for example, as identified by the alignment in FIG. 20A. In specific embodiments, the Aβ antigen binding fragment comprises a light chain comprising an amino acid sequence of SEQ ID NO: 2 with 1, 2, 3, 4, 5, 6, 7, 8, 9, 10, 11, 12, 13, 14, 15 or more amino acid substitutions, insertions or deletions, and the substitutions, insertions or deletions are made for example, in the framework regions (e.g., those regions outside of the CDRs, which CDRs are underlined in FIG. 2A) or are substitutions with an amino acid present at that position in the light chain of one or more of the other therapeutic antibodies, for example, as identified by the alignment in FIG. 20B.

In certain embodiments, the anti-Aβ antigen-binding fragment transgene encodes a hyperglycosylated solanezumab Fab, comprising a heavy chain and a light chain of SEQ ID NOs: 1 and 2, respectively, with one or more of the following mutations: L107N (heavy chain), Q165N or Q165S (light chain), and/or E200N (light chain) (see FIG. 20A (heavy chain) and B (light chain)).

In certain embodiments, the anti-Aβ antigen-binding fragment transgene encodes an antigen-binding fragment and comprises the nucleotide sequences encoding the six solanezumab CDRs which are underlined in the heavy and light chain variable domain sequences of FIG. 2A which are spaced between framework regions, generally human framework regions, and associated with constant domains depending upon the form of the antigen-binding molecule, as is known in the art to form the heavy and/or light chain variable domain of an anti-Aβ antibody or antigen-binding fragment thereof.

In certain embodiments, the anti-Aβ antigen-binding fragment transgene comprises the nucleotide sequences encoding the heavy and light chains of the Fab portion of GSK933776 (having amino acid sequences of SEQ ID NOs. 3 and 4, respectively, see Table 5 and FIG. 2B). The nucleotide sequences may be codon optimized for expression in human cells. Nucleotide sequences may, for example, comprise the nucleotide sequences of SEQ ID NO: 73 (encoding the GSK933776 heavy chain Fab portion) and SEQ ID NO: 74 (encoding the GSK933776 light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or a signal sequence found in Table 2.

In addition to the heavy and light chain variable domain and C_{H}1 and C_{L} sequences, the transgenes may comprise, at the C-terminus of the heavy chain C_{H}1 sequence, all or a portion of the hinge region. In specific embodiments, the anti-Aβ-antigen binding domain has a heavy chain Fab domain of SEQ ID NO: 3 with additional hinge region sequence starting after the C-terminal valine (V), contains all or a portion of the amino acid sequence EPKSCDKTHTCPPCPAPELAGA (SEQ ID NO: 202), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELAGAPSVFL (SEQ ID NO: 204) or EPKSCDKTHLCPPCPAPELAGAPSVFL (SEQ ID NO: 205) as set forth in FIG. 2B. These hinge regions may be encoded by nucleotide sequences at the 3′ end of SEQ ID NO: 3 by the hinge region encoding sequences set forth in Table 6 (SEQ ID NO: 73). In another embodiment, the transgenes comprise the amino acid sequences encoding the full length (or substantially full length) heavy and light chains of the antibody, comprising the Fc domain at the C terminus of the heavy chain, e.g., an IgG1 Fc domain, such as SEQ ID NO. 291 (Table 7), or alternatively, SEQ ID No. 283 or as depicted in FIG. 23, or a mutant or variant thereof. In particular embodiments, the Fc domain has alanine substitutions at positions 235 and 237 (EU numbering). The Fc domain may be engineered for altered binding to one or more Fc receptors and/or effector function as disclosed in Section 5.1.9, infra.

In certain embodiments, the anti-Aβ antigen-binding fragment transgene encodes an Aβ antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-Aβ antigen-binding fragment transgene encodes an Aβ antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, the anti-Aβ antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In specific embodiments, the Aβ antigen binding fragment comprises a heavy chain comprising an amino acid sequence of SEQ ID NO: 3 with 1, 2, 3, 4, 5, 6, 7, 8, 9, 10, 11, 12, 13, 14, 15 or more amino acid substitutions, insertions or deletions, and the substitutions, insertions or deletions, e.g., are made in the framework regions (e.g., those regions outside of the CDRs, which CDRs are underlined in FIG. 2B) or are substitutions with an amino acid present at that position in the heavy chain of one or more of the other therapeutic antibodies, for example, as identified by the alignment in FIG. 20A. In specific embodiments, the Aβ antigen binding fragment comprises a light chain comprising an amino acid sequence of SEQ ID NO: 4 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2B) or are substitutions with an amino acid present at that position in the light chain of one or more of the other therapeutic antibodies, for example, as identified by the alignment in FIG. 20B.

In certain embodiments, the anti-Aβ antigen-binding fragment transgene encodes a hyperglycosylated GSK933776 Fab, comprising a heavy chain and a light chain of SEQ ID NOs: 3 and 4, respectively, with one or more of the following mutations: L110N (heavy chain), Q165N or Q165S (light chain), and/or E200N (light chain) (see FIG. 20A (heavy chain) and B (light chain)).

In certain embodiments, the anti-Aβ antigen-binding fragment transgene encodes an antigen-binding fragment and comprises the nucleotide sequences encoding the six GSK933776 CDRs which are underlined in the heavy and light chain variable domain sequences of FIG. 2B which are spaced between framework regions, generally human framework regions, and associated with constant domains depending upon the form of the antigen-binding molecule, as is known in the art to form the heavy and/or light chain variable domain of an anti-Aβ antibody or antigen-binding fragment thereof.

In certain embodiments, the anti-Aβ antigen-binding fragment transgene comprises the nucleotide sequences encoding the heavy and light chains of the Fab portion of lecanemab (having amino acid sequences of SEQ ID NOs. 360 and 361, respectively, see Table 5 and FIG. 2C). The nucleotide sequences may be codon optimized for expression in human cells. Nucleotide sequences may, for example, comprise the nucleotide sequences of SEQ ID NO: 376 (encoding the lecanemab heavy chain Fab portion) and SEQ ID NO: 377 (encoding the lecanemab light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the Fab fragments, including the heavy and light chain variable domain sequences and the C_{L} and C_{H}1 domains, the transgenes may comprise, at the C-terminus of the heavy chain C_{H}1 sequence, all or a portion of the hinge region. In specific embodiments, the anti-Aβ-antigen binding domain has a heavy chain variable domain and C_{H}1 domain of SEQ ID NO: 360 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGG (SEQ ID NO: 200) or EPKSCDKTHLCPPCPAPELLGGPSVFL (SEQ ID NO: 201) as set forth in FIG. 2C. These hinge regions may be encoded by nucleotide sequences at the 3′ end of SEQ ID NO: 376 by the hinge region encoding sequences set forth in Table 6 (SEQ ID NO: 376). In another embodiment, the transgenes comprise the amino acid sequences encoding the full length (or substantially full length) heavy and light chains of the antibody, comprising the Fc domain at the C terminus of the heavy chain, e.g. an IgG1 Fc domain, such as SEQ ID NO: 392 of Table 7, SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-Aβ antigen-binding fragment transgene encodes an Aβ antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 361. In certain embodiments, the anti-Aβ antigen-binding fragment transgene encodes an Aβ antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 360. In certain embodiments, the anti-Aβ antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 361 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 360. In specific embodiments, the Aβ antigen binding fragment comprises a heavy chain comprising an amino acid sequence of SEQ ID NO: 360 with 1, 2, 3, 4, 5, 6, 7, 8, 9, 10, 11, 12, 13, 14, 15 or more amino acid substitutions, insertions or deletions, and the substitutions, insertions or deletions are made for example, in the framework regions (e.g., those regions outside of the CDRs, which CDRs are underlined in FIG. 2C) or are substitutions with an amino acid present at that position in the heavy chain of one or more of the other therapeutic antibodies, for example, as identified by the alignment in FIG. 20A. In specific embodiments, the Aβ antigen binding fragment comprises a light chain comprising an amino acid sequence of SEQ ID NO: 361 with 1, 2, 3, 4, 5, 6, 7, 8, 9, 10, 11, 12, 13, 14, 15 or more amino acid substitutions, insertions or deletions, and the substitutions, insertions or deletions are made for example, in the framework regions (e.g., those regions outside of the CDRs, which CDRs are underlined in FIG. 2C) or are substitutions with an amino acid present at that position in the light chain of one or more of the other therapeutic antibodies, for example, as identified by the alignment in FIG. 20B.

In certain embodiments, the anti-Aβ antigen-binding fragment transgene encodes a hyperglycosylated lecanemab Fab, comprising a heavy chain and a light chain of SEQ ID NOs: 360 and 361, respectively, with one or more of the following mutations: T119N (heavy chain), Q165N or Q165S (light chain), and/or E200N (light chain) (see FIG. 20A (heavy chain) and B (light chain)).

In certain embodiments, the anti-Aβ antigen-binding fragment transgene encodes an antigen-binding fragment and comprises the nucleotide sequences encoding the six lecanemab CDRs which are underlined in the heavy and light chain variable domain sequences of FIG. 2C which are spaced between framework regions, generally human framework regions, and associated with constant domains depending upon the form of the antigen-binding molecule, as is known in the art to form the heavy and/or light chain variable domain of an anti-Aβ antibody or antigen-binding fragment thereof.

Gene Therapy Methods

Provided are methods of treating human subjects for AD by administration of a viral vector containing a transgene encoding an anti-Aβ antibody, or antigen binding fragment thereof. The antibody may be solanezumab, lecanemab, or GSK933776 and is, e.g., a Fab fragment thereof, or other antigen-binding fragment thereof. In other embodiments, the antibody is a full-length or substantially full-length antibody having an Fc region. In certain embodiments, the patient has been diagnosed with and/or has symptoms associated with prodromal AD, e.g., a mild cognitive impairment associated with early AD or even pre-AD. Recombinant vectors used for delivering the transgene are described in Section 5.4.1. Such vectors should have a tropism for human CNS cells and can include non-replicating rAAV, particularly those bearing an AAV9, AAVrh10, AAVrh20, AAVrh39, or AAVcy5 capsid. The recombinant vectors can be administered in any manner such that the recombinant vector enters the CNS, e.g., by introducing the recombinant vector into the cerebral spinal fluid (CSF). See Section 5.5.1 for details regarding the methods of treatment.

Subjects to whom such gene therapy is administered can be those responsive to anti-Aβ therapy. In particular embodiments, the methods encompass treating patients who have been diagnosed with AD, or have one or more symptoms associated therewith, and identified as responsive to treatment with an anti-Aβ antibody or considered a good candidate for therapy with an anti-Aβ antibody. In specific embodiments, the patients have previously been treated with solanezumab, lecanemab, or GSK933776 and have been found to be responsive to solanezumab, lecanemab, and/or GSK933776. To determine responsiveness, the anti-Aβ antibody or antigen-binding fragment transgene product (e.g., produced in human cell culture, bioreactors, etc.) may be administered directly to the subject.

Human Post Translationally Modified Antibodies

The production of the anti-Aβ HuPTM mAb or HuPTM Fab, should result in a “biobetter” molecule for the treatment of AD accomplished via gene therapy—e.g., by administering a viral vector or other DNA expression construct encoding the anti-Aβ HuPTM Fab or HuPTM mAb, intrathecally, particularly intracisternal or lumbar administration, or intravenous administration to human subjects (patients) diagnosed with or having one or more symptoms of AD, to create a permanent depot in the CNS that continuously supplies the fully-human post-translationally modified, e.g., human-glycosylated, sulfated transgene product produced by transduced CNS cells.

The cDNA construct for the anti-Aβ HuPTMmAb or anti-An HuPTM Fab should include a signal peptide that ensures proper co- and post-translational processing (glycosylation and protein sulfation) by the transduced CNS cells. For example, the signal sequence may be MYRMQLLLLIALSLALVTNS (SEQ ID NO: 146).

As an alternative, or an additional treatment to gene therapy, the anti-An HuPTM mAb or HuPTM Fab can be produced in human cell lines by recombinant DNA technology, and administered to patients diagnosed with AD, or for whom therapy for AD is considered appropriate.

In specific embodiments, the anti-An HuPTM mAb or antigen-binding fragment thereof has heavy and light chains with the amino acid sequences of the heavy and light chain Fab portions of solanezumab as set forth in FIG. 2A (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N56, Q104, and/or N154 of the heavy chain (SEQ ID NO:1) or Q105, N163 and/or N215 of the light chain (SEQ ID NO: 2). Alternatively, or in addition to, the HuPTM mAb or antigen binding-fragment thereof with the heavy and light chain variable domain sequences of solanezumab has a sulfation group at Y94, Y95 and/or Y101 of the heavy chain (SEQ ID NO: 1) or Y91 and/or Y92 of the light chain (SEQ ID NO: 2). In other embodiments, the anti-Aβ HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the anti-Aβ-HuPTM mAb is a full length mAb with an Fc region, e.g., the Fc domain of SEQ ID NO 290, or alternatively of SEQ ID NO: 283, SEQ ID NO: 284 or SEQ ID NO: 285, or a mutant or variant thereof.

In specific embodiments, the anti-An HuPTM mAb or antigen-binding fragment thereof has heavy and light chains with the amino acid sequences of the heavy and light chain Fab portions of GSK933776 as set forth in FIG. 2B (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N32, Q107, and/or N157 of the heavy chain (SEQ ID NO: 3) or N163 and/or N215 of the light chain (SEQ ID NO: 4). Alternatively or in addition to, the HuPTM mAb or antigen binding-fragment thereof with the heavy and light chain variable domain sequences of GSK933776 has a sulfation group at Y94 and/or Y95 of the heavy chain (SEQ ID NO: 3) or Y91 and/or Y92 of the light chain (SEQ ID NO: 4). In other embodiments, the anti-Aβ HuPTM mAb or antigen-binding fragment thereof does not contain any detectable NeuGc moieties and/or does not contain any detectable alpha-Gal moieties. In certain embodiments, the anti-Aβ-HuPTM mAb is a full length mAb with an Fc region, e.g., the Fc domain of SEQ ID NO: 291, or alternatively SEQ ID NO: 283, SEQ ID NO: 284 or SEQ ID NO: 285, or a mutant or variant thereof, and, e.g., has alanine substitutions at positions 235 and 237 (EU numbering).

In specific embodiments, the anti-Aβ HuPTM mAb or antigen-binding fragment thereof has heavy and light chains with the amino acid sequences of the heavy and light chain Fab portions of lecanemab as set forth in FIG. 2C (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions Q116, and/or N166 of the heavy chain (SEQ ID NO: 360) or N163 and/or N215 of the light chain (SEQ ID NO: 361). Alternatively, or in addition to, the HuPTM mAb or antigen binding-fragment thereof with the heavy and light chain variable domain sequences of lecanemab has a sulfation group at Y94 and/or Y95 of the heavy chain (SEQ ID NO: 360) or Y91 and/or Y92 of the light chain (SEQ ID NO: 361). In other embodiments, the anti-Aβ HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the anti-Aβ-HuPTM mAb is a full length mAb with an Fc region, e.g., the Fc domain of SEQ ID NO 392, or alternatively of SEQ ID NO: 283 or a mutant or variant thereof. In certain embodiments, the HuPTM mAb or Fab is therapeutically effective and is at least 0.5%, 1% or 2% 2,6 sialylated and/or sulfated and may be at least 5%, 10% or even 50% or 100% glycosylated 2,6 sialylation and/or sulfated. The goal of gene therapy treatment provided herein is to slow or arrest the progression of AD, particular cognitive impairment. Efficacy may be monitored by measuring a reduction in plaque formation and/or an improvement in cognitive function or a reduction in the decline in cognitive function.

Combinations of delivery of the anti-Aβ HuPTM mAb or antigen-binding fragment thereof, to the CNS accompanied by delivery of other available treatments are encompassed by the methods provided herein. The additional treatments may be administered before, concurrently or subsequent to the gene therapy treatment. Available treatments for AD that could be combined with the gene therapy provided herein include but are not limited to ARICEPT® (donepezil), RAZADYNE® (galantamine), NAMENDA® (rivastigmine), and NAMZARIC® (donepezil and memantine), to name a few, and administration with anti-Aβ agents, including but not limited to solanezumab, GSK933776, or lecanemab, or anti-Tau agents, such as aTAU.

### 5.3.2. Anti-Sortilin HuPTM Constructs and Formulations for Frontotemporal Dementia

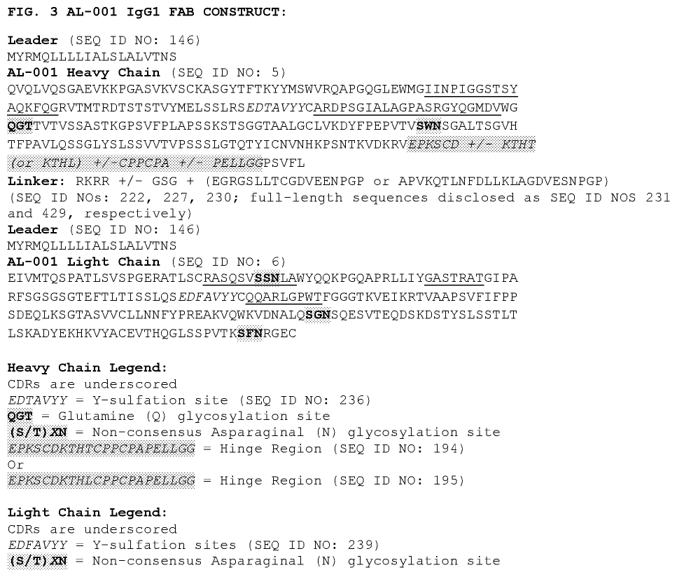

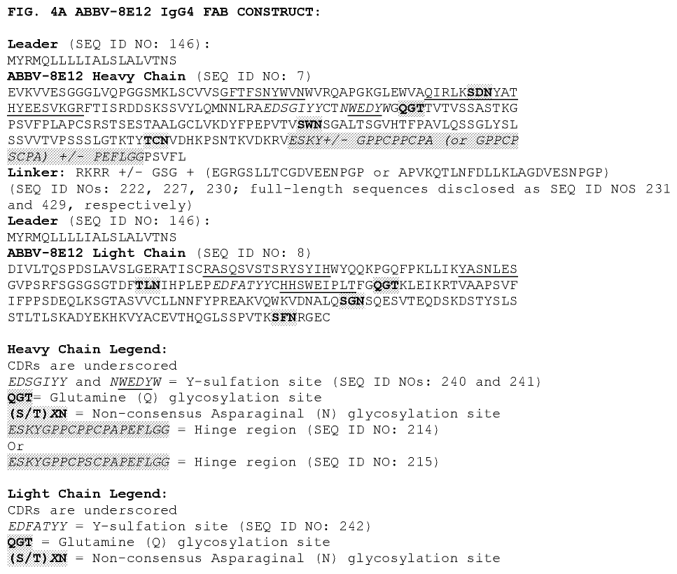

Compositions and methods are described for the delivery of HuPTM mAbs and antigen-binding fragments thereof, such as HuPTM Fabs, that bind to sortilin that may have benefit in treating frontotemporal dementia (FD). In particular embodiments, the HuPTM mAb is AL-001, or an antigen binding fragment of AL-001. The amino acid sequences of the heavy and light chains of Fab fragments of this antibody is provided in FIG. 3. Delivery may be accomplished via gene therapy—e.g., by administering a viral vector or other DNA expression construct encoding a sortilin-binding HuPTM mAb (or an antigen binding fragment and/or a hyperglycosylated derivative or other derivative, thereof) to patients (human subjects) diagnosed with, or having one or more symptoms of, FD, to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to sortilin that can be administered to deliver the HuPTM mAb or antigen binding fragment in a patient. The transgene is a nucleic acid comprising the nucleotide sequences encoding an antigen binding fragment of an antibody that binds to sortilin, such as AL-001, or variants thereof as detailed herein. The transgene may also encode an anti-sortilin antigen binding fragment that contains additional glycosylation sites (e.g., see Courtois et al., 2016, mAbs 8: 99-112 which is incorporated by reference herein in its entirety).

In certain embodiments, the anti-sortilin antigen-binding fragment transgene comprises the nucleotide sequences encoding the heavy and light chains of the Fab portion of AL-001 (having amino acid sequences of SEQ ID NOs. 5 and 6, respectively, see Table 5 and FIG. 3). The nucleotide sequences may be codon optimized for expression in human cells. Nucleotide sequences may, for example, comprise the nucleotide sequences of SEQ ID NO: 75 (encoding the AL-001 heavy chain Fab portion) and SEQ ID NO: 76 (encoding the AL-001 light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2, supra.

In addition to the heavy and light chain variable domain sequences and the C_{H}1 and C_{L} domains, the transgenes may comprise, at the C-terminus sequence, all or a portion of the hinge region. In specific embodiments, the anti-sortilin-antigen binding domain has a heavy chain Fab domain of SEQ ID NO: 5 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 3. These hinge regions may be encoded by nucleotide sequences at the 3′ end of SEQ ID NO: 75 by the hinge region encoding sequences set forth in Table 6 (SEQ ID NO: 75). In another embodiment, the transgenes comprise the amino acid sequences encoding the full length (or substantially full length) heavy and light chains of the antibody, comprising the Fc domain at the C terminus of the heavy chain, e.g., an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-sortilin antigen-binding fragment transgene encodes an sortilin antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 6. In certain embodiments, the anti-sortilin antigen-binding fragment transgene encodes an sortilin antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 5. In certain embodiments, the anti-sortilin antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 6 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 5. In specific embodiments, the sortilin antigen binding fragment comprises a heavy chain comprising an amino acid sequence of SEQ ID NO: 5 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIG. 3) or are substitutions with an amino acid present at that position in the heavy chain of one or more of the other therapeutic antibodies, for example, as identified by the alignment in FIG. 20A. In specific embodiments, the sortilin antigen binding fragment comprises a light chain comprising an amino acid sequence of SEQ ID NO: 6 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3) or are substitutions with an amino acid present at that position in the light chain of one or more of the other therapeutic antibodies, for example, as identified by the alignment in FIG. 20B.

In certain embodiments, the anti-sortilin antigen-binding fragment transgene encodes a hyperglycosylated AL-001 Fab or mAb, comprising a heavy chain and a light chain of SEQ ID NOs: 5 and 6, respectively, with one or more of the following mutations: T124N (heavy chain), Q160N or Q160S (light chain), and/or E199N (light chain) (see FIG. 20A (heavy chain) and B (light chain)).

In certain embodiments, the anti-sortilin antigen-binding fragment transgene encodes an antigen-binding fragment and comprises the nucleotide sequences encoding the six AL-001 CDRs which are underlined in the heavy and light chain variable domain sequences of FIG. 3 which are spaced between framework regions, generally human framework regions, and associated with constant domains depending upon the form of the antigen-binding molecule, as is known in the art to form the heavy and/or light chain variable domain of an anti-sortilin antibody or antigen-binding fragment thereof.

Gene Therapy Methods

Provided are methods of treating human subjects for FD by administration of a viral vector containing a transgene encoding an anti-sortilin antibody, or antigen binding fragment thereof. The antibody may be AL-001 and is, e.g., a Fab fragment thereof, or other antigen-binding fragment thereof. In certain embodiments, the transgene encodes a full length or substantially full-length Al-001 mAb, including the Fc region. In certain embodiments, the patient has been diagnosed with FD and/or has symptoms associated with FD or prodromal FD, e.g., a mild cognitive impairment associated with early FD or even pre-FD. Recombinant vectors used for delivering the transgene are described in Section 5.4.1. Such vectors should have a tropism for human CNS cells and can include non-replicating rAAV, particularly those bearing an AAV9, AAVrh10, AAVrh20, AAVrh39, or AAVcy5 capsid. The recombinant vectors can be administered in any manner such that the recombinant vector enters the CNS, e.g., by introducing the recombinant vector into the cerebral spinal fluid (CSF). See Section 5.5.1 for details regarding the methods of treatment.

Subjects to whom such gene therapy is administered can be those responsive to anti-sortilin therapy. In particular embodiments, the methods encompass treating patients who have been diagnosed with FD, or have one or more symptoms associated therewith, and identified as responsive to treatment with an anti-sortilin antibody or considered a good candidate for therapy with an anti-sortilin antibody. In specific embodiments, the patients have previously been treated with AL-001, and have been found to be responsive to AL-001. To determine responsiveness, the anti-sortilin antibody or antigen-binding fragment transgene product (e.g., produced in human cell culture, bioreactors, etc.) may be administered directly to the subject.

Human Post Translationally Modified Antibodies

The production of the anti-sortilin HuPTM mAb or HuPTM Fab, should result in a “biobetter” molecule for the treatment of FD accomplished via gene therapy—e.g., by administering a viral vector or other DNA expression construct encoding the anti-sortilin HuPTM Fab or HuPTM mAb, intrathecally, particularly intracisternal or lumbar administration, or intravenous administration to human subjects (patients) diagnosed with or having one or more symptoms of FD, to create a permanent depot in the CNS that continuously supplies the fully-human post-translationally modified, e.g., human-glycosylated, sulfated transgene product produced by transduced CNS cells.

The cDNA construct for the anti-sortilin HuPTM mAb or anti-sortilin HuPTM Fab should include a signal peptide that ensures proper co- and post-translational processing (glycosylation and protein sulfation) by the transduced CNS cells. For example, the signal sequence may be MYRMQLLLLIALSLALVTNS (SEQ ID NO: 146).

As an alternative, or an additional treatment to gene therapy, the anti-sortilin HuPTM mAb or HuPTM Fab can be produced in human cell lines by recombinant DNA technology, and administered to patients diagnosed with FD, or for whom therapy for FD is considered appropriate.

In specific embodiments, the anti-sortilin HuPTM mAb or antigen-binding fragment thereof has heavy and light chains with the amino acid sequences of the heavy and light chain Fab portions of AL-001 as set forth in FIG. 3 (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions Q121 and/or N171 of the heavy chain (SEQ ID NO:5) or N32, N158 and/or N210 of the light chain (SEQ ID NO: 6). Alternatively or in addition to, the HuPTM mAb or antigen binding-fragment thereof with the heavy and light chain variable domain sequences of AL-001 has a sulfation group at Y94 and/or Y95 of the heavy chain (SEQ ID NO: 5) or Y86 and/or Y87 of the light chain (SEQ ID NO: 6). In other embodiments, the anti-sortilin HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the anti-sortilin-HuPTM mAb is a full length mAb with an Fc region, e.g., the Fc domain of SEQ ID NO: 283, SEQ ID NO: 284 or SEQ ID NO: 285, or a mutant or variant thereof.

In certain embodiments, the HuPTM mAb or Fab is therapeutically effective and is at least 0.5%, 1% or 2% 2,6 sialylated and/or sulfated and may be at least 5%, 10% or even 50% or 100% glycosylated 2,6 sialylation and/or sulfated. The goal of gene therapy treatment provided herein is to slow or arrest the progression of FD, particular cognitive impairment. Efficacy may be monitored by measuring an improvement in cognitive function and/or a reduction in the deterioration in behavior, personality and/or difficulty with producing or comprehending language.

Combinations of delivery of the anti-sortilin HuPTM mAb or antigen-binding fragment thereof, to the CNS accompanied by delivery of other available treatments are encompassed by the methods provided herein. The additional treatments may be administered before, concurrently or subsequent to the gene therapy treatment. Available treatments for FD that could be combined with the gene therapy provided herein.

### 5.3.3. Anti-Tau HuPTM Constructs and Formulations for Tauopathies Like Alzheimer's Disease, Chronic Traumatic Encephalopathy, Progressive Supranuclear Palsy, or Frontotemporal Dementia

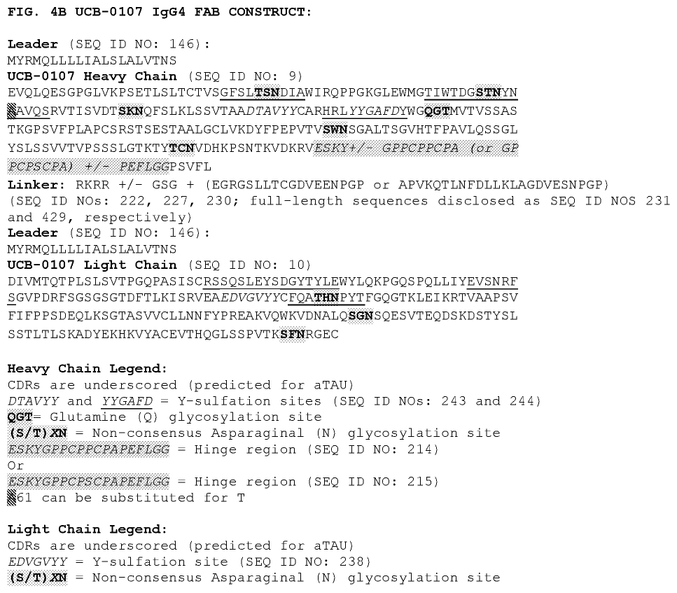

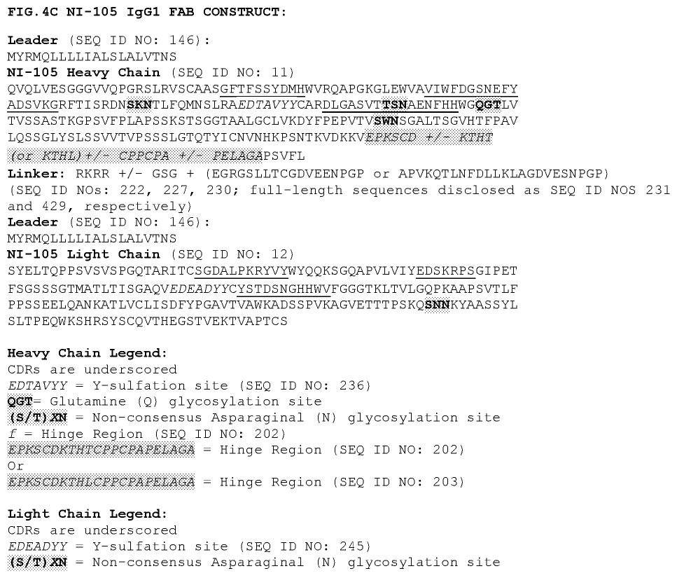

Compositions and methods are described for the delivery of HuPTM mAbs and antigen-binding fragments thereof, such as HuPTM Fabs, that bind to Tau protein (Tau), such as monomeric Tau, oligomeric Tau, non-phosphorylated Tau, and phosphorylated Tau, that may have benefit in treating Alzheimer's Disease (AD), Chronic Traumatic Encephalopathy (CTE), Pick's Complex, primary age-related tauopathy, progressive supranuclear palsy (PSP), FD, and other tauopathies. In particular embodiments, the HuPTM mAb is ABBV-8E12, UCB-0107, and NI-105 (BIIB076), or an antigen binding fragment of one of the foregoing. The amino acid sequences of Fab fragments of these antibodies are provided in FIGS. 4A-C. Delivery may be accomplished via gene therapy—e.g., by administering a viral vector or other DNA expression construct encoding a Tau-binding HuPTM mAb (or an antigen binding fragment and/or a hyperglycosylated derivative or other derivative, thereof) to patients (human subjects) diagnosed with, or having one or more symptoms of, AD, CTE, PSP, FD, or other tauopathies, to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to Tau that can be administered to deliver the HuPTM mAb or antigen binding fragment in a patient. The transgene is a nucleic acid comprising the nucleotide sequences encoding an antigen binding fragment of an antibody that binds to Tau, such as ABBV-8E12, UCB-0107, and NI-105 (BIIB076), or variants there of as detailed herein. The transgene may also encode anti-Tau antigen binding fragment that contains additional glycosylation sites (e.g., see Courtois et al., 2016, mAbs 8: 99-112 which is incorporated by reference herein in its entirety).

In certain embodiments, the anti-Tau antigen-binding fragment transgene comprises the nucleotide sequences encoding the heavy and light chains of the Fab portion of ABBV-8E12 (having amino acid sequences of SEQ ID NOs. 7 and 8, respectively, see Table 5 and FIG. 4A). The nucleotide sequences may be codon optimized for expression in human cells. Nucleotide sequences may, for example, comprise the nucleotide sequences of SEQ ID NO: 77 (encoding the ABBV-8E12 heavy chain Fab portion) and SEQ ID NO: 78 (encoding the ABBV-8E12 light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2, supra.

In addition to the heavy and light chain variable domain and C_{H}1 and C_{L} sequences, the transgenes may comprise, at the C-terminus of the C_{H}1 domain, all or a portion of the hinge region. In specific embodiments, the anti-Tau-antigen binding domain has a heavy chain Fab domain of SEQ ID NO: 7 with additional hinge region sequence starting after the C-terminal tyrosine (Y), contains all or a portion of the amino acid sequence ESKYGPPCPPCPAPEFLGG (SEQ ID NO: 214), and specifically, ESKYGPPCPPCPA (SEQ ID NO: 216), ESKYGPPCPSCPA (SEQ ID NO: 217), ESKYGPPCPSCPAPEFLGGPSVFL (SEQ ID NO: 218), or ESKYGPPCPPCPAPEFLGGPSVFL (SEQ ID NO: 219) as set forth in FIG. 4A. These hinge regions may be encoded by nucleotide sequences at the 3′ end of SEQ ID NO: 77 by the hinge region encoding sequences set forth in Table 5 (SEQ ID NO: 77). In another embodiment, the transgenes comprise the amino acid sequences encoding the full length (or substantially full length) heavy and light chains of the antibody, comprising the Fc domain at the C terminus of the heavy chain, e.g., an IgG1 or IgG4 Fc domain, such as SEQ ID Nos. 283 or 285, including with a S241P substitution (EU numbering) or as depicted in FIG. 23, or a mutant or variant thereof. The Fc domain may be engineered for altered binding to one or more Fc receptors and/or effector function as disclosed in Section 5.1.9, infra.

In certain embodiments, the anti-Tau antigen-binding fragment transgene encodes a Tau antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 8. In certain embodiments, the anti-Tau antigen-binding fragment transgene encodes a Tau antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 7. In certain embodiments, the anti-Tau antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 8 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 7. In specific embodiments, the Tau antigen-binding fragment comprises a heavy chain comprising an amino acid sequence of SEQ ID NO: 7 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 4A) or are substitutions with an amino acid present at that position in the heavy chain of one or more of the other therapeutic antibodies, for example, as identified by the alignment in FIG. 20A. In specific embodiments, the Tau antigen binding fragment comprises a light chain comprising an amino acid sequence of SEQ ID NO: 8 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 4A) or are substitutions with an amino acid present at that position in the light chain of one or more of the other therapeutic antibodies, for example, as identified by the alignment in FIG. 20B.

In certain embodiments, the anti-Tau antigen-binding fragment transgene encodes a hyperglycosylated ABBV-8E12 Fab, comprising a heavy chain and a light chain of SEQ ID NOs: 7 and 8, respectively, with one or more of the following mutations: T110N (heavy chain), Q164N or Q164S (light chain), and/or E199N (light chain) (see FIG. 20A (heavy chain) and B (light chain)).

In certain embodiments, the anti-Tau antigen-binding fragment transgene encodes an antigen-binding fragment and comprises the nucleotide sequences encoding the six ABBV-8E12 CDRs which are underlined in the heavy and light chain variable domain sequences of FIG. 4A which are spaced between framework regions, generally human framework regions, and associated with constant domains depending upon the form of the antigen-binding molecule, as is known in the art to form the heavy and/or light chain variable domain of an anti-Tau antibody or antigen-binding fragment thereof.

In certain embodiments, the anti-Tau antigen-binding fragment transgene comprises the nucleotide sequences encoding the heavy and light chains of the Fab portion of UCB-0107 (having amino acid sequences of SEQ ID NOs. 9 and 10, respectively, see Table 5 and FIG. 4B). The nucleotide sequences may be codon optimized for expression in human cells. Nucleotide sequences may, for example, comprise the nucleotide sequences of SEQ ID NO: 79 (encoding the UCB-0107 heavy chain Fab portion) and SEQ ID NO: 80 (encoding the UCB-0107 light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain, C_{H}1 and C_{L} domain sequences, the transgenes may comprise, at the C-terminus of the heavy chain C_{H}1 domain sequence, all or a portion of the hinge region. In specific embodiments, the anti-Tau-antigen binding domain has a heavy chain Fab fragment of SEQ ID NO: 9 with additional hinge region sequence starting after the C-terminal valine (V), contains all or a portion of the amino acid sequence ESKYGPPCPPCPAPEFLGG (SEQ ID NO: 214), and specifically, ESKYGPPCPPCPA (SEQ ID NO: 216), ESKYGPPCPSCPA (SEQ ID NO: 217), ESKYGPPCPSCPAPEFLGGPSVFL (SEQ ID NO: 218), or ESKYGPPCPPCPAPEFLGGPSVFL (SEQ ID NO: 219) as set forth in FIG. 4B. These hinge regions may be encoded by nucleotide sequences at the 3′ end of SEQ ID NO: 79 by the hinge region encoding sequences set forth in Table 5 (SEQ ID NO: 79). In another embodiment, the transgenes comprise the amino acid sequences encoding the full length (or substantially full length) heavy and light chains of the antibody, comprising the Fc domain at the C terminus of the heavy chain, e.g., SEQ ID NO. 292 (Table 7), or an IgG4 Fc domain, such as SEQ ID No. 285 or as depicted in FIG. 23, or a mutant or variant thereof. The Fc domain may be engineered for altered binding to one or more Fc receptors and/or effector function as disclosed in Section 5.1.9, infra.

In certain embodiments, the anti-Tau antigen-binding fragment transgene encodes a Tau antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 10. In certain embodiments, the anti-Tau antigen-binding fragment transgene encodes a Tau antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 9. In certain embodiments, the anti-Tau antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 10 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 9. In specific embodiments, the Tau antigen-binding fragment comprises a heavy chain comprising an amino acid sequence of SEQ ID NO: 9 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 4B) or are substitutions with an amino acid present at that position in the heavy chain of one or more of the other therapeutic antibodies, for example, as identified by the alignment in FIG. 20A. In specific embodiments, the Tau antigen binding fragment comprises a light chain comprising an amino acid sequence of SEQ ID NO: 10 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 4B) or are substitutions with an amino acid present at that position in the light chain of one or more of the other therapeutic antibodies, for example, as identified by the alignment in FIG. 20B.

In certain embodiments, the anti-Tau antigen-binding fragment transgene encodes a hyperglycosylated UCB-0107 Fab, comprising a heavy chain and a light chain of SEQ ID NOs: 9 and 10, respectively, with one or more of the following mutations: M113N (heavy chain), Q165N or Q165S (light chain), and/or E200N (light chain) (see FIG. 20A (heavy chain) and B (light chain)).

In certain embodiments, the anti-Tau antigen-binding fragment transgene encodes an antigen-binding fragment and comprises the nucleotide sequences encoding the six UCB-0107 CDRs which are underlined in the heavy and light chain variable domain sequences of FIG. 4B which are spaced between framework regions, generally human framework regions, and associated with constant domains depending upon the form of the antigen-binding molecule, as is known in the art to form the heavy and/or light chain variable domain of an anti-Tau antibody or antigen-binding fragment thereof.

In certain embodiments, the anti-Tau antigen-binding fragment transgene comprises the nucleotide sequences encoding the heavy and light chains of the Fab portion of NI-105 (having amino acid sequences of SEQ ID NOs. 11 and 12, respectively, see Table 5 and FIG. 4C). The nucleotide sequences may be codon optimized for expression in human cells. Nucleotide sequences may, for example, comprise the nucleotide sequences of SEQ ID NO: 81 (encoding the NI-105 heavy chain Fab portion) and SEQ ID NO: 82 (encoding the NI-105 light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain and C_{H}1 and C_{L} sequences, the transgenes may comprise, at the C-terminus of the C_{H}1 domain sequence, all or a portion of the hinge region. In specific embodiments, the anti-Tau-antigen binding domain has a heavy chain Fab fragment of SEQ ID NO: 11 with additional hinge region sequence starting after the C-terminal valine (V), contains all or a portion of the amino acid sequence EPKSCDKTHTCPPCPAPELAGA (SEQ ID NO: 202), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELAGAPSVFL (SEQ ID NO: 204) or EPKSCDKTHLCPPCPAPELAGAPSVFL (SEQ ID NO: 205) as set forth in FIG. 4C. These hinge regions may be encoded by nucleotide sequences at the 3′ end of SEQ ID NO: 81 by the hinge region encoding sequences set forth in Table 5 (SEQ ID NO: 81). In another embodiment, the transgenes comprise the amino acid sequences encoding the full length (or substantially full length) heavy and light chains of the antibody, comprising the Fc domain at the C terminus of the heavy chain, e.g., an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-Tau antigen-binding fragment transgene encodes a Tau antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 12. In certain embodiments, the anti-Tau antigen-binding fragment transgene encodes a Tau antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 11. In certain embodiments, the anti-Tau antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 12 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 11. In specific embodiments, the Tau antigen-binding fragment comprises a heavy chain comprising an amino acid sequence of SEQ ID NO: 11 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 4C) or are substitutions with an amino acid present at that position in the heavy chain of one or more of the other therapeutic antibodies, for example, as identified by the alignment in FIG. 20A. In specific embodiments, the Tau antigen binding fragment comprises a light chain comprising an amino acid sequence of SEQ ID NO: 12 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 4C) or are substitutions with an amino acid present at that position in the light chain of one or more of the other therapeutic antibodies, for example, as identified by the alignment in FIG. 20B.

In certain embodiments, the anti-Tau antigen-binding fragment transgene encodes a hyperglycosylated NI-105 Fab, comprising a heavy chain and a light chain of SEQ ID NOs: 11 and 12, respectively, with one or more of the following mutations: L119N (heavy chain) and/or Q196N (light chain) (see FIG. 20A (heavy chain) and B (light chain)).

In certain embodiments, the anti-Tau antigen-binding fragment transgene encodes an antigen-binding fragment and comprises the nucleotide sequences encoding the six NI-105 CDRs which are underlined in the heavy and light chain variable domain sequences of FIG. 4C which are spaced between framework regions, generally human framework regions, and associated with constant domains depending upon the form of the antigen-binding molecule, as is known in the art to form the heavy and/or light chain variable domain of an anti-Tau antibody or antigen-binding fragment thereof.

Gene Therapy Methods

Provided are methods of treating human subjects for AD, CTE, PSP, FD, or other tauopathies by administration of a viral vector containing a transgene encoding an anti-Tau antibody, or antigen binding fragment thereof. The antibody may be ABBV-8E12, UCB-0107, or NI-105 (BIIB076), and is, for example, a Fab fragment thereof, or other antigen-binding fragment thereof. In certain embodiments, the antibody is a full length or substantially full length mAb with Fc region. In certain embodiments, the patient has been diagnosed with and/or has symptoms associated with prodromal AD, e.g., a mild cognitive impairment associated with early AD or even pre-AD. Recombinant vectors used for delivering the transgene are described in Section 5.4.1. Such vectors should have a tropism for human CNS cells and can include non-replicating rAAV, particularly those bearing an AAV9, AAVrh10, AAVrh20, AAVrh39, or AAVcy5 capsid. The recombinant vectors can be administered in any manner such that the recombinant vector enters the CNS, e.g., by introducing the recombinant vector into the cerebral spinal fluid (CSF). See Section 5.5.1 for details regarding the methods of treatment.

Subjects to whom such gene therapy is administered can be those responsive to anti-Tau therapy. In particular embodiments, the methods encompass treating patients who have been diagnosed with AD, PSP, or FD, or have one or more symptoms associated therewith, and identified as responsive to treatment with an anti-Tau antibody or considered a good candidate for therapy with an anti-Tau antibody. In specific embodiments, the patients have previously been treated with ABBV-8E12, UCB-0107, and/or NI-105 (BIIB076), and have been found to be responsive to ABBV-8E12, UCB-0107, and/or NI-105 (BIIB076). To determine responsiveness, the anti-Tau antibody or antigen-binding fragment transgene product (e.g., produced in human cell culture, bioreactors, etc.) may be administered directly to the subject.

Human Post Translationally Modified Antibodies

The production of the anti-Tau HuPTM mAb or HuPTM Fab, should result in a “biobetter” molecule for the treatment of AD, PSP, or FD accomplished via gene therapy—e.g., by administering a viral vector or other DNA expression construct encoding the anti-Tau HuPTM Fab or HuPTM mAb, intrathecally, particularly intracisternal or lumbar administration, or intravenous administration to human subjects (patients) diagnosed with or having one or more symptoms of AD, PSP, or FD, to create a permanent depot in the CNS that continuously supplies the fully-human post-translationally modified, e.g., human-glycosylated, sulfated transgene product produced by transduced CNS cells.

The cDNA construct for the anti-Tau HuPTMmAb or anti-Tau HuPTM Fab should include a signal peptide that ensures proper co- and post-translational processing (glycosylation and protein sulfation) by the transduced CNS cells. For example, the signal sequence may be MYRMQLLLLIALSLALVTNS (SEQ ID NO: 146).

As an alternative, or an additional treatment to gene therapy, the anti-Tau HuPTM mAb or HuPTM Fab can be produced in human cell lines by recombinant DNA technology, and administered to patients diagnosed with AD, PSP, or FD, or for whom therapy for AD, PSP, or FD is considered appropriate.

In specific embodiments, the anti-Tau HuPTM mAb or antigen-binding fragment thereof has heavy and light chains with the amino acid sequences of the heavy and light chain Fab portions of ABBV-8E12 as set forth in FIG. 4A (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N57, Q107, N157 and/or N199 of the heavy chain (SEQ ID NO: 7) or N78, Q104, N162, and/or N214 of the light chain (SEQ ID NO: 8). Alternatively or in addition to, the HuPTM mAb or antigen binding-fragment thereof with the heavy and light chain variable domain sequences of ABBV-8E12 has a sulfation group at Y96, Y97 and/or Y104 of the heavy chain (SEQ ID NO: 7) and/or Y90 and/or Y91 of the light chain (SEQ ID NO: 8). In other embodiments, the anti-Tau HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In specific embodiments, the anti-Tau HuPTM mAb or antigen-binding fragment thereof has heavy and light chains with the amino acid sequences of the heavy and light chain Fab portions of UCB-0107 as set forth in FIG. 4B (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N32, N58, N76, Q110, N160 and/or N202 of the heavy chain (SEQ ID NO: 9) or N99, N163 and/or N215 of the light chain (SEQ ID NO: 10). Alternatively or in addition to, the HuPTM mAb or antigen binding-fragment thereof with the heavy and light chain variable domain sequences of UCB-0107 has a sulfation group at Y93, Y94, Y101 and/or Y102 of the heavy chain (SEQ ID NO: 9) and/or Y91 and/or Y92 of the light chain (SEQ ID NO: 10). In other embodiments, the anti-Tau HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In specific embodiments, the anti-Tau HuPTM mAb or antigen-binding fragment thereof has heavy and light chains with the amino acid sequences of the heavy and light chain Fab portions of NI-105 as set forth in FIG. 4C (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N77, N107, Q116, and/or N166 of the heavy chain (SEQ ID NO: 11) and/or N172 of the light chain (SEQ ID NO: 12). Alternatively or in addition to, the HuPTM mAb or antigen binding-fragment thereof with the heavy and light chain variable domain sequences of NI-105 has a sulfation group at Y93 and/or Y94 of the heavy chain (SEQ ID NO: 11) and/or Y85 and/or Y86 of the light chain (SEQ ID NO: 12). In other embodiments, the anti-Tau HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% 2,6 sialylated and/or sulfated and may be at least 5%, 10% or even 50% or 100% glycosylated 2,6 sialylation and/or sulfated. The goal of gene therapy treatment provided herein is to slow or arrest the progression of AD, PSP, or FD, particularly cognitive impairment, gross or fine motor skill impairment, or vision impairment. Efficacy may be monitored by measuring a reduction in plaque formation and/or an improvement in cognitive function, with motor skills, or with vision or a reduction in the decline in cognitive function, motor skills, or vision.

Combinations of delivery of the anti-Tau HuPTM mAb or antigen-binding fragment thereof, to the CNS accompanied by delivery of other available treatments are encompassed by the methods provided herein. The additional treatments may be administered before, concurrently or subsequent to the gene therapy treatment. Available treatments for AD, PSP, or FD that could be combined with the gene therapy provided herein include but are not limited to ARICEPT® (donepezil), RAZADYNE® (galantamine), NAMENDA® (rivastigmine), and NAMZARIC® (donepezil and memantine), to name a few, and administration with anti-Tau agents, including but not limited to anti-tau, such as, but not limited to ABBV-8E12, UCB-0107, or NI-105, and anti-An agents, such as, but not limited to solanezumab, lecanemab, or GSK933776.

### 5.3.4. Anti-SEMA4D HuPTM Constructs and Formulations for Huntington's Disease

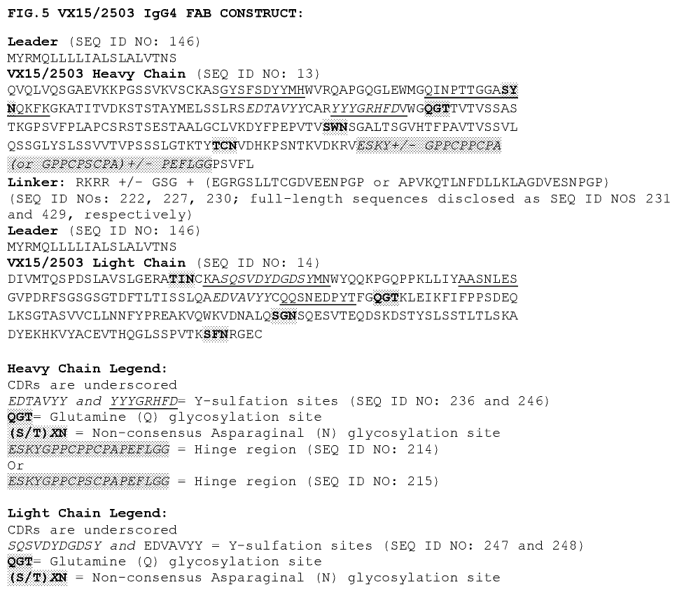

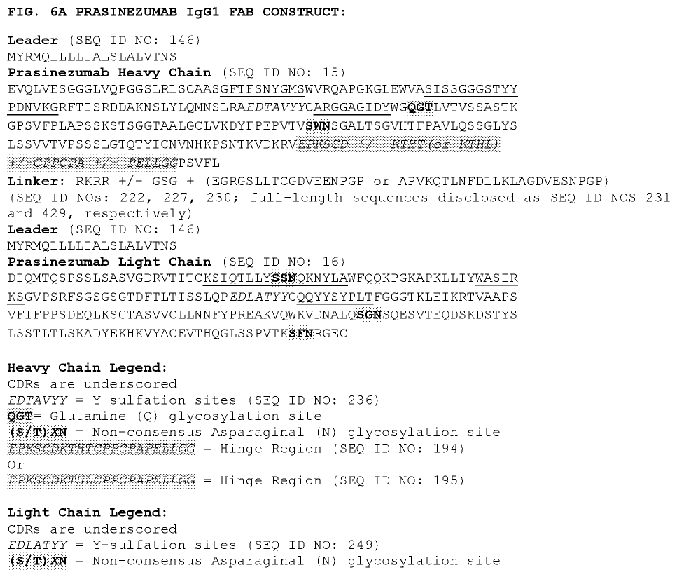

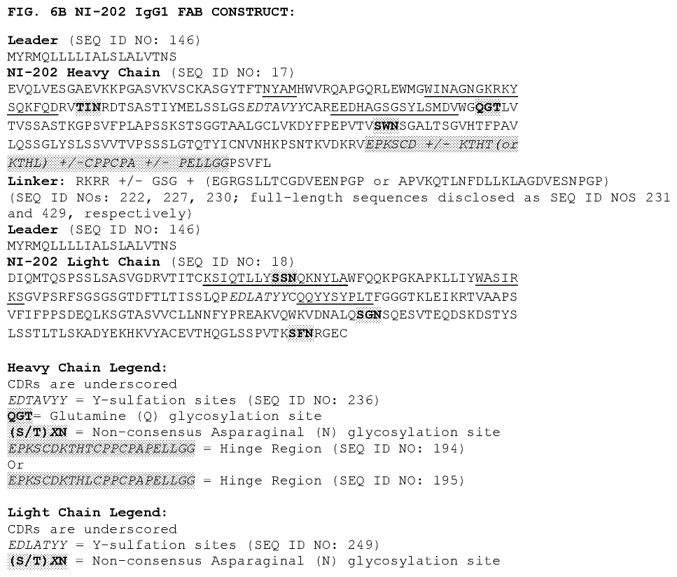

Compositions and methods are described for the delivery of HuPTM mAbs and antigen-binding fragments thereof, such as HuPTM Fabs, that bind to semaphorin 4D (SEMA4D) that may have benefit in treating Huntington's disease (HD) and juvenile Huntington's disease (JHD). In particular embodiments, the HuPTM mAb is VX15/2503, or an antigen binding fragment of VX15/2503. The amino acid sequences of Fab fragments of this antibody is provided in FIG. 5. Delivery may be accomplished via gene therapy—e.g., by administering a viral vector or other DNA expression construct encoding an SEMA4D-binding HuPTM mAb (or an antigen binding fragment and/or a hyperglycosylated derivative or other derivative, thereof) to patients (human subjects) diagnosed with, or having one or more symptoms of, HD, to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to SEMA4D that can be administered to deliver the HuPTM mAb or antigen binding fragment in a patient. The transgene is a nucleic acid comprising the nucleotide sequences encoding an antigen binding fragment of an antibody that binds to SEMA4D, such as VX15/2503, or variants there of as detailed herein. The transgene may also encode an anti-SEMA4D antigen binding fragment that contains additional glycosylation sites (e.g., see Courtois et al., 2016, mAbs 8: 99-112 which is incorporated by reference herein in its entirety).

In certain embodiments, the anti-SEMA4D antigen-binding fragment transgene comprises the nucleotide sequences encoding the heavy and light chains of the Fab portion of VX15/2503 (having amino acid sequences of SEQ ID NOs. 13 and 14, respectively, see Table 5 and FIG. 5). The nucleotide sequences may be codon optimized for expression in human cells. Nucleotide sequences may, for example, comprise the nucleotide sequences of SEQ ID NO: 83 (encoding the VX15/2503 heavy chain Fab portion) and SEQ ID NO: 84 (encoding the VX15/2503 light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain and C_{H}1 and C_{L} sequences, the transgenes may comprise, at the C-terminus of the heavy chain C_{H}1 sequence, all or a portion of the hinge region. In specific embodiments, the anti-SEMA4D-antigen binding domain has a heavy chain variable domain and C_{H}1 domain of SEQ ID NO: 13 with additional hinge region sequence starting after the C-terminal valine (V), contains all or a portion of the amino acid sequence contains all or a portion of the amino acid sequence ESKYGPPCPPCPAPEFLGG (SEQ ID NO: 214), and specifically, ESKYGPPCPPCPA (SEQ ID NO: 216), ESKYGPPCPSCPA (SEQ ID NO: 217), ESKYGPPCPSCPAPEFLGGPSVFL (SEQ ID NO: 218), or ESKYGPPCPPCPAPEFLGGPSVFL (SEQ ID NO: 219) as set forth in FIG. 5. These hinge regions may be encoded by nucleotide sequences at the 3′ end of SEQ ID NO: 83 by the hinge region encoding sequences set forth in Table 6 (SEQ ID NO: 83). In another embodiment, the transgenes comprise the amino acid sequences encoding the full length (or substantially full length) heavy and light chains of the antibody, comprising the Fc domain at the C terminus of the heavy chain, e.g., an IgG4 Fc domain, such as SEQ ID No. 285 or as depicted in FIG. 23, or a mutant or variant thereof. The Fc domain may be engineered for altered binding to one or more Fc receptors and/or effector function as disclosed in Section 5.1.9, infra.

In certain embodiments, the anti-SEMA4D antigen-binding fragment transgene encodes an SEMA4D antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 14. In certain embodiments, the anti-SEMA4D antigen-binding fragment transgene encodes an SEMA4D antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 13. In certain embodiments, the anti-SEMA4D antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 14 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 13. In specific embodiments, the SEMA4D antigen binding fragment comprises a heavy chain comprising an amino acid sequence of SEQ ID NO: 13 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 5) or are substitutions with an amino acid present at that position in the heavy chain of one or more of the other therapeutic antibodies, for example, as identified by the alignment in FIG. 20A. In specific embodiments, the SEMA4D antigen binding fragment comprises a light chain comprising an amino acid sequence of SEQ ID NO: 14 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 5) or are substitutions with an amino acid present at that position in the light chain of one or more of the other therapeutic antibodies, for example, as identified by the alignment in FIG. 20B.

In certain embodiments, the anti-SEMA4D antigen-binding fragment transgene encodes a hyperglycosylated VX15/2503 Fab, comprising a heavy chain and a light chain of SEQ ID NOs: 13 and 14, respectively, with one or more of the following mutations: T113N (heavy chain), Q156N or Q156S (light chain), and/or E191N (light chain) (see FIG. 20A (heavy chain) and B (light chain)).

In certain embodiments, the anti-SEMA4D antigen-binding fragment transgene encodes an antigen-binding fragment and comprises the nucleotide sequences encoding the six VX15/2503 CDRs which are underlined in the heavy and light chain variable domain sequences of FIG. 5 which are spaced between framework regions, generally human framework regions, and associated with constant domains depending upon the form of the antigen-binding molecule, as is known in the art to form the heavy and/or light chain variable domain of an anti-SEMA4D antibody or antigen-binding fragment thereof.

Gene Therapy Methods

Provided are methods of treating human subjects for HD or juvenile HD by administration of a viral vector containing a transgene encoding an anti-SEMA4D antibody, or antigen binding fragment thereof. The antibody may be VX15/2503 and is, e.g., a Fab fragment thereof, or other antigen-binding fragment thereof. In certain embodiments, the transgene encodes the full length or substantially full length VX15/2503. In certain embodiments, the patient has been diagnosed with and/or has symptoms associated with HD, e.g., mild involuntary movements, tremors, and/or dystonia associated with early HD or even pre-HD. Recombinant vectors used for delivering the transgene are described in Section 5.4.1. Such vectors should have a tropism for human CNS cells and can include non-replicating rAAV, particularly those bearing an AAV9, AAVrh10, AAVrh20, AAVrh39, or AAVcy5 capsid. The recombinant vectors can be administered in any manner such that the recombinant vector enters the CNS, e.g., by introducing the recombinant vector into the cerebral spinal fluid (CSF). See Section 5.5.1 for details regarding the methods of treatment.

Subjects to whom such gene therapy is administered can be those responsive to anti-SEMA4D therapy. In particular embodiments, the methods encompass treating patients who have been diagnosed with HD, or have one or more symptoms associated therewith, and identified as responsive to treatment with an anti-SEMA4D antibody or considered a good candidate for therapy with an anti-SEMA4D antibody. In specific embodiments, the patients have previously been treated with VX15/2503, and have been found to be responsive to VX15/2503. To determine responsiveness, the anti-SEMA4D antibody or antigen-binding fragment transgene product (e.g., produced in human cell culture, bioreactors, etc.) may be administered directly to the subject.

Human Post Translationally Modified Antibodies

The production of the anti-SEMA4D HuPTM mAb or HuPTM Fab, should result in a “biobetter” molecule for the treatment of HD accomplished via gene therapy—e.g., by administering a viral vector or other DNA expression construct encoding the anti-SEMA4D HuPTM Fab, intrathecally, particularly intracisternal or lumbar administration, or intravenous administration to human subjects (patients) diagnosed with or having one or more symptoms of HD, to create a permanent depot in the CNS that continuously supplies the fully-human post-translationally modified, e.g., human-glycosylated, sulfated transgene product produced by transduced CNS cells.

The cDNA construct for the anti-SEMA4D HuPTM mAb or anti-SEMA4D HuPTM Fab should include a signal peptide that ensures proper co- and post-translational processing (glycosylation and protein sulfation) by the transduced CNS cells. For example, the signal sequence may be MYRMQLLLLIALSLALVTNS (SEQ ID NO: 146).

As an alternative, or an additional treatment to gene therapy, the anti-SEMA4D HuPTM mAb or HuPTM Fab can be produced in human cell lines by recombinant DNA technology and administered to patients diagnosed with HD or juvenile HD, or for whom therapy for HD or juvenile HD is considered appropriate.

In specific embodiments, the anti-SEMA4D HuPTM mAb or antigen-binding fragment thereof has heavy and light chains with the amino acid sequences of the heavy and light chain Fab portions of VX15/2503 as set forth in FIG. 5 (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N61, Q110, N160 and/or N207 of the heavy chain (SEQ ID NO:13) or N22, Q104, N154 and/or N206 of the light chain (SEQ ID NO: 14). Alternatively or in addition to, the HuPTM mAb or antigen binding-fragment thereof with the heavy and light chain variable domain sequences of VX15/2503 has a sulfation group at Y94, Y95, Y99, Y100, and/or Y101 of the heavy chain (SEQ ID NO: 13) or Y31, Y36, Y90, and/or Y91 of the light chain (SEQ ID NO: 14). In other embodiments, the anti-SEMA4D HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% 2,6 sialylated and/or sulfated and may be at least 5%, 10% or even 50% or 100% glycosylated 2,6 sialylation and/or sulfated. The goal of gene therapy treatment provided herein is to slow or arrest the progression of HD or juvenile HD, particular the impairment in voluntary movements. Efficacy may be monitored by measuring improvements in movement, dystonia, and/or an improvement in cognitive function or a reduction in the decline in chorea control and cognitive function. In the case of juvenile HD, efficacy may be monitored by measuring improvements in muscle stiffness, dystonia, and/or chorea or a reduction in the decline in muscle and cognitive function.

Combinations of delivery of the anti-SEMA4D HuPTM mAb or antigen-binding fragment thereof, to the CNS accompanied by delivery of other available treatments are encompassed by the methods provided herein. The additional treatments may be administered before, concurrently or subsequent to the gene therapy treatment. Available treatments for HD or juvenile HD that could be combined with the gene therapy provided herein include but are not limited to speech, physical, and occupational therapy, XENAZINE® (Tetrabenazine), KLONOPIN® (clonazepam), HALDOL® (haloperidol), CLORAZIL® (clozapine), PROZAD® (fluoxetine), ZOLOFT® (sertraline), and PAMELOR® (nortriptyline), and administration with anti-SEMA4D agents, including but not limited to VX15/2503.

### 5.3.5. Anti-Alpha-Synuclein HuPTM Constructs and Formulations for Parkinson and Synucleinopathies

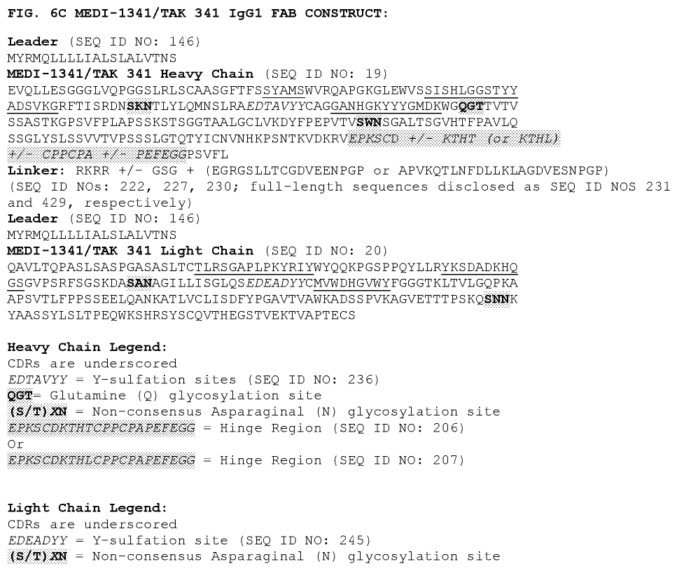

Compositions and methods are described for the delivery of HuPTM mAbs and antigen-binding fragments thereof, such as HuPTM Fabs, that bind to alpha-synuclein (SNCA) that may have benefit in treating Parkinson disease (PD) and other synucleinopathies such as dementia with Lewy bodies (DLB), pure autonomic failure (PAF), and multiple system atrophy (MSA). In particular embodiments, the HuPTM mAb is prasinezumab, NI-202 (BIIB054), and MED-1341, or an antigen binding fragment of one of the foregoing. The amino acid sequences of Fab fragments of these antibodies are provided in FIGS. 6A-C. Delivery may be accomplished via gene therapy—e.g., by administering a viral vector or other DNA expression construct encoding a SNCA-binding HuPTM mAb (or an antigen binding fragment and/or a hyperglycosylated derivative or other derivative, thereof) to patients (human subjects) diagnosed with, or having one or more symptoms of, PD, DLB, PAF, MSA, to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to SNCA that can be administered to deliver the HuPTM mAb or antigen binding fragment in a patient. The transgene is a nucleic acid comprising the nucleotide sequences encoding an antigen binding fragment of an antibody that binds to SNCA, such as prasinezumab, NI-202 (BIIB054), or MED-1341, or variants there of as detailed herein. The transgene may also encode anti-SNCA antigen binding fragment that contains additional glycosylation sites (e.g., see Courtois et al., 2016, mAbs 8: 99-112 which is incorporated by reference herein in its entirety).

In certain embodiments, the anti-SNCA antigen-binding fragment transgene comprises the nucleotide sequences encoding the heavy and light chains of the Fab portion of prasinezumab (having amino acid sequences of SEQ ID NOs. 15 and 16, respectively, see Table 5 and FIG. 6A). The nucleotide sequences may be codon optimized for expression in human cells. Nucleotide sequences may, for example, comprise the nucleotide sequences of SEQ ID NO: 85 (encoding the prasinezumab heavy chain Fab portion) and SEQ ID NO: 86 (encoding the prasinezumab light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain and C_{H}1 and C_{L} domain sequences, the transgenes may comprise, at the C-terminus of the heavy chain C_{H}1 sequence, all or a portion of the hinge region. In specific embodiments, the anti-SNCA-antigen binding domain has a heavy chain Fab fragment of SEQ ID NO: 15 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 6A. These hinge regions may be encoded by nucleotide sequences at the 3′ end of SEQ ID NO: 85 by the hinge region encoding sequences set forth in Table 5 (SEQ ID NO: 85). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 293 (Table 7), or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-SNCA antigen-binding fragment transgene encodes a SNCA antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 16. In certain embodiments, the anti-SNCA antigen-binding fragment transgene encodes a SNCA antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 15. In certain embodiments, the anti-SNCA antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 16 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 15. In specific embodiments, the SNCA antigen-binding fragment comprises a heavy chain comprising an amino acid sequence of SEQ ID NO: 16 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 6A) or are substitutions with an amino acid present at that position in the heavy chain of one or more of the other therapeutic antibodies, for example, as identified by the alignment in FIG. 20A. In specific embodiments, the SNCA antigen binding fragment comprises a light chain comprising an amino acid sequence of SEQ ID NO: 16 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 6A) or are substitutions with an amino acid present at that position in the light chain of one or more of the other therapeutic antibodies, for example, as identified by the alignment in FIG. 20B.

In certain embodiments, the anti-Tau antigen-binding fragment transgene encodes a hyperglycosylated prasinezumab Fab, comprising a heavy chain and a light chain of SEQ ID NOs: 15 and 16, respectively, with one or more of the following mutations: L119N (heavy chain), Q166N or Q166S (light chain), and/or E201N (light chain) (see FIG. 20A (heavy chain) and B (light chain)).

In certain embodiments, the anti-SNCA antigen-binding fragment transgene encodes an antigen-binding fragment and comprises the nucleotide sequences encoding the six prasinezumab CDRs which are underlined in the heavy and light chain variable domain sequences of FIG. 6A which are spaced between framework regions, generally human framework regions, and associated with constant domains depending upon the form of the antigen-binding molecule, as is known in the art to form the heavy and/or light chain variable domain of an anti-SNCA antibody or antigen-binding fragment thereof.

In certain embodiments, the anti-SNCA antigen-binding fragment transgene comprises the nucleotide sequences encoding the heavy and light chains of the Fab portion of NI-202 (having amino acid sequences of SEQ ID NOs. 17 and 18, respectively, see Table 5 and FIG. 6B). The nucleotide sequences may be codon optimized for expression in human cells. Nucleotide sequences may, for example, comprise the nucleotide sequences of SEQ ID NO: 87 (encoding the NI-202 heavy chain Fab portion) and SEQ ID NO: 88 (encoding the NI-202 light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-SNCA-antigen binding domain has a heavy chain Fab fragment of SEQ ID NO: 17 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 6B. These hinge regions may be encoded by nucleotide sequences at the 3′ end of SEQ ID NO: 87 by the hinge region encoding sequences set forth in Table 5 (SEQ ID NO: 87). In another embodiment, the transgenes comprise the amino acid sequences encoding the full length (or substantially full length) heavy and light chains of the antibody, comprising the Fc domain at the C terminus of the heavy chain, e.g., an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-SNCA antigen-binding fragment transgene encodes a SNCA antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 18. In certain embodiments, the anti-SNCA antigen-binding fragment transgene encodes a SNCA antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 17. In certain embodiments, the anti-SNCA antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 18 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 17. In specific embodiments, the SNCA antigen-binding fragment comprises a heavy chain comprising an amino acid sequence of SEQ ID NO: 17 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 6B) or are substitutions with an amino acid present at that position in the heavy chain of one or more of the other therapeutic antibodies, for example, as identified by the alignment in FIG. 20A. In specific embodiments, the SNCA antigen binding fragment comprises a light chain comprising an amino acid sequence of SEQ ID NO: 18 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 6B) or are substitutions with an amino acid present at that position in the light chain of one or more of the other therapeutic antibodies, for example, as identified by the alignment in FIG. 20B.

In certain embodiments, the anti-SNCA antigen-binding fragment transgene encodes a hyperglycosylated NI-202 Fab, comprising a heavy chain and a light chain of SEQ ID NOs: 17 and 18, respectively, with one or more of the following mutations: L119N (heavy chain), Q166N or Q166S (light chain), and/or E199N (light chain) (see FIG. 20A (heavy chain) and B (light chain)).

In certain embodiments, the anti-SNCA antigen-binding fragment transgene encodes an antigen-binding fragment and comprises the nucleotide sequences encoding the six NI-202 CDRs which are underlined in the heavy and light chain variable domain sequences of FIG. 6B which are spaced between framework regions, generally human framework regions, and associated with constant domains depending upon the form of the antigen-binding molecule, as is known in the art to form the heavy and/or light chain variable domain of an anti-SNCA antibody or antigen-binding fragment thereof.

In certain embodiments, the anti-SNCA antigen-binding fragment transgene comprises the nucleotide sequences encoding the heavy and light chains of the Fab portion of MED-1341 (having amino acid sequences of SEQ ID NOs. 19 and 20, respectively, see Table 5 and FIG. 6C). The nucleotide sequences may be codon optimized for expression in human cells. Nucleotide sequences may, for example, comprise the nucleotide sequences of SEQ ID NO: 89 (encoding the MEDI-1341 heavy chain Fab portion) and SEQ ID NO: 90 (encoding the MEDI-1341 light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain and C_{H}1 or C_{L} sequences, the transgenes may comprise, at the C-terminus of the heavy chain C_{H}1 sequence, all or a portion of the hinge region. In specific embodiments, the anti-SNCA-antigen binding domain has a heavy chain Fab fragment of SEQ ID NO: 19 with additional hinge region sequence starting after the C-terminal valine (V), contains all or a portion of the amino acid sequence EPKSCDKTHTCPPCPAPEFEGG (SEQ ID NO: 206), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPEFEGGPSVFL (SEQ ID NO: 208) or EPKSCDKTHLCPPCPAPEFEGGPSVFL (SEQ ID NO: 209) as set forth in FIG. 6C. These hinge regions may be encoded by nucleotide sequences at the 3′ end of SEQ ID NO: 89 by the hinge region encoding sequences set forth in Table 6 (SEQ ID NO: 89). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 294 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-SNCA antigen-binding fragment transgene encodes a SNCA antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 20. In certain embodiments, the anti-SNCA antigen-binding fragment transgene encodes a SNCA antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 19. In certain embodiments, the anti-SNCA antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 20 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 19. In specific embodiments, the SNCA antigen-binding fragment comprises a heavy chain comprising an amino acid sequence of SEQ ID NO: 19 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 6C) or are substitutions with an amino acid present at that position in the heavy chain of one or more of the other therapeutic antibodies, for example, as identified by the alignment in FIG. 20A. In specific embodiments, the SNCA antigen binding fragment comprises a light chain comprising an amino acid sequence of SEQ ID NO: 20 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 6C) or are substitutions with an amino acid present at that position in the light chain of one or more of the other therapeutic antibodies, for example, as identified by the alignment in FIG. 20B.

In certain embodiments, the anti-SNCA antigen-binding fragment transgene encodes a hyperglycosylated MEDI-1341 Fab, comprising a heavy chain and a light chain of SEQ ID NOs: 19 and 20, respectively, with one or more of the following mutations: T117N (heavy chain) and/or Q203N (light chain) (see FIG. 20A (heavy chain) and B (light chain)).

In certain embodiments, the anti-SNCA antigen-binding fragment transgene encodes an antigen-binding fragment and comprises the nucleotide sequences encoding the six MEDI-1341 CDRs which are underlined in the heavy and light chain variable domain sequences of FIG. 6C which are spaced between framework regions, generally human framework regions, and associated with constant domains depending upon the form of the antigen-binding molecule, as is known in the art to form the heavy and/or light chain variable domain of an anti-SNCA antibody or antigen-binding fragment thereof.

Gene Therapy Methods

Provided are methods of treating human subjects for PD, DLB, PAF, MSA, or other synulceinopathies by administration of a viral vector containing a transgene encoding an anti-SNCA antibody, or antigen binding fragment thereof. The antibody may be prasinezumab, NI-202 (BIIB054), or MED-1341, and is e.g. a Fab fragment thereof, or other antigen-binding fragment thereof. In other embodiments, the transgene encodes a full length or substantially full-length antibody with Fc region. In certain embodiments, the patient has been diagnosed with and/or has symptoms associated with PD or other synulceinopathies, e.g., a mild cognitive and/or motor skill impairment associated with early PD or even pre-PD. Recombinant vectors used for delivering the transgene are described in Section 5.4.1. Such vectors should have a tropism for human CNS cells and can include non-replicating rAAV, particularly those bearing an AAV9, AAVrh10, AAVrh20, AAVrh39, or AAVcy5 capsid. The recombinant vectors can be administered in any manner such that the recombinant vector enters the CNS, e.g., by introducing the recombinant vector into the cerebral spinal fluid (CSF). See Section 5.5.1 for details regarding the methods of treatment.

Subjects to whom such gene therapy is administered can be those responsive to anti-SNCA therapy. In certain embodiments, the methods encompass treating patients who have been diagnosed with PD, DLB, PAF, or MSA, or have one or more symptoms associated therewith, and identified as responsive to treatment with an anti-SNCA antibody or considered a good candidate for therapy with an anti-SNCA antibody. In specific embodiments, the patients have previously been treated with prasinezumab, NI-202 (B1113054) and/or MED-1341, and have been found to be responsive to prasinezumab, NI-202 (BIIB054) and/or MED-1341. To determine responsiveness, the anti-SNCA antibody or antigen-binding fragment transgene product (e.g., produced in human cell culture, bioreactors, etc.) may be administered directly to the subject.

Human Post Translationally Modified Antibodies

The production of the anti-SNCA HuPTM mAb or HuPTM Fab, should result in a “biobetter” molecule for the treatment of PD, DLB, PAF, or MSA accomplished via gene therapy—e.g., by administering a viral vector or other DNA expression construct encoding the anti-SNCA HuPTM Fab or HuPTM mAb, intrathecally, particularly intracisternal or lumbar administration, or intravenous administration to human subjects (patients) diagnosed with or having one or more symptoms of PD, DLB, PAF, or MSA, to create a permanent depot in the CNS that continuously supplies the fully-human post-translationally modified, e.g., human-glycosylated, sulfated transgene product produced by transduced CNS cells.

The cDNA construct for the anti-SNCA HuPTMmAb or anti-SNCA HuPTM Fab should include a signal peptide that ensures proper co- and post-translational processing (glycosylation and protein sulfation) by the transduced CNS cells. For example, the signal sequence may be MYRMQLLLLIALSLALVTNS (SEQ ID NO: 146).

As an alternative, or an additional treatment to gene therapy, the anti-SNCA HuPTM mAb or HuPTM Fab can be produced in human cell lines by recombinant DNA technology, and administered to patients diagnosed with PD, DLB, PAF, or MSA or for whom therapy for PD, DLB, PAF or MSA is considered appropriate.

In specific embodiments, the anti-SNCA HuPTM mAb or antigen-binding fragment thereof has heavy and light chains with the amino acid sequences of the heavy and light chain Fab portions of prasinezumab as set forth in FIG. 6A (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions Q108 and/or N158 of the heavy chain (SEQ ID NO: 15) or N34, N164, and/or N216 of the light chain (SEQ ID NO: 16). Alternatively or in addition to, the HuPTM mAb or antigen binding-fragment thereof with the heavy and light chain variable domain sequences of prasinezumab has a sulfation group at Y94 and/or Y95 of the heavy chain (SEQ ID NO: 15) and/or Y92 and/or Y93 of the light chain (SEQ ID NO: 16). In other embodiments, the anti-SNCA HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In specific embodiments, the anti-SNCA HuPTM mAb or antigen-binding fragment thereof has heavy and light chains with the amino acid sequences of the heavy and light chain Fab portions of NI-202 as set forth in FIG. 6B (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N71, Q116, and/or N166 of the heavy chain (SEQ ID NO: 17) or N34, N164 and/or N216 of the light chain (SEQ ID NO: 18). Alternatively or in addition to, the HuPTM mAb or antigen binding-fragment thereof with the heavy and light chain variable domain sequences of NI-202 has a sulfation group at Y94 and/or Y95 of the heavy chain (SEQ ID NO: 17) or Y92 and/or Y93 of the light chain (SEQ ID NO: 18). In other embodiments, the anti-SNCA HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In specific embodiments, the anti-SNCA HuPTM mAb or antigen-binding fragment thereof has heavy and light chains with the amino acid sequences of the heavy and light chain Fab portions of MEDI-1341 as set forth in FIG. 6C (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N77, Q114, and/or N164 of the heavy chain (SEQ ID NO: 19) or N172 of the light chain (SEQ ID NO: 20). Alternatively or in addition to, the HuPTM mAb or antigen binding-fragment thereof with the heavy and light chain variable domain sequences of MED-1341 has a sulfation group at Y94 and/or Y95 of the heavy chain (SEQ ID NO: 19) or Y94 and/or Y95 of the light chain (SEQ ID NO: 20). In other embodiments, the anti-SNCA HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% 2,6 sialylated and/or sulfated and may be at least 5%, 10% or even 50% or 100% glycosylated 2,6 sialylation and/or sulfated. The goal of gene therapy treatment provided herein is to slow or arrest the progression of PD, DLB, PFA, or MSP, particularly cognitive impairment, gross or fine motor skill impairment, or vision impairment. Efficacy may be monitored by measuring an improvement in cognitive function, motor skills (i.e. posture, balance, tremor), and/or vision or a reduction in the decline in cognitive function, motor skills, or vision.

Combinations of delivery of the anti-SNCA HuPTM mAb or antigen-binding fragment thereof, to the CNS accompanied by delivery of other available treatments are encompassed by the methods provided herein. The additional treatments may be administered before, concurrently or subsequent to the gene therapy treatment. Available treatments for PD, DLB, PFA, or MSP that could be combined with the gene therapy provided herein include but are not limited to RYTARY®, SINEMET®, or DUOPA® (carbidopa/levodopa), to name a few, and administration with anti-SNCA agents, including but not limited to anti-SNCA, such as, but not limited to prasinezumab, NI-202 (BIIB054), or MED-1341.

### 5.3.6. Anti-SOD1 HuPTM Constructs and Formulations for Alzheimer's Disease and Amyotrophic Lateral Sclerosis

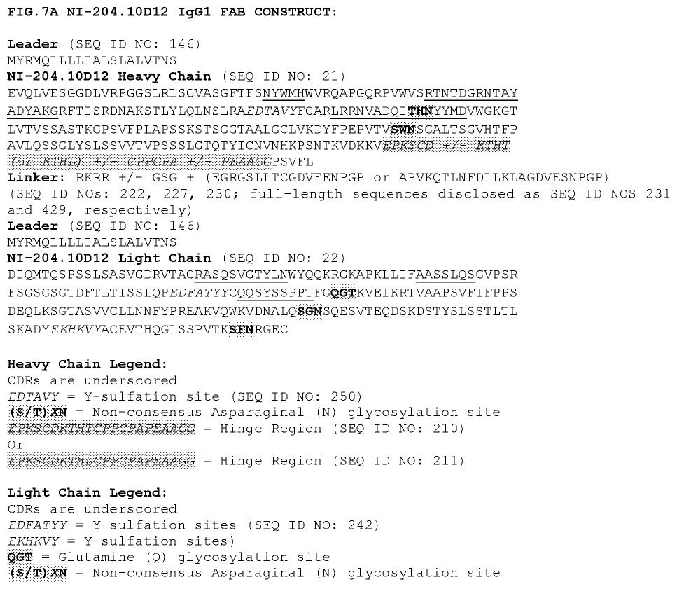

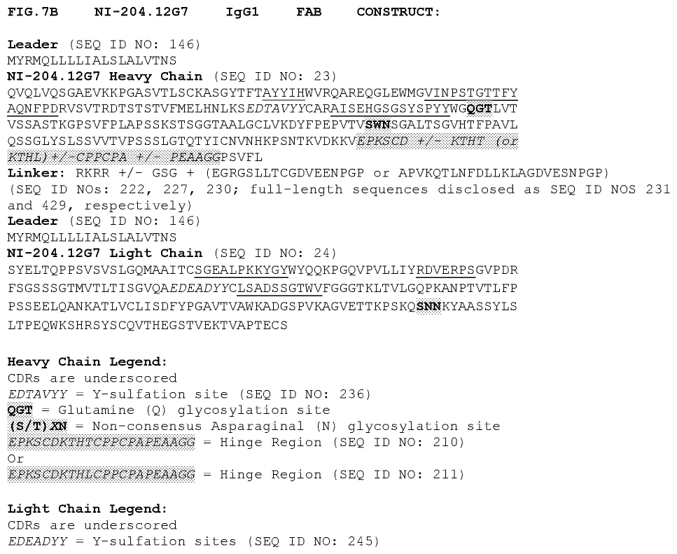

Compositions and methods are described for the delivery of HuPTM mAbs and antigen-binding fragments thereof, such as HuPTM Fabs, that bind to superoxide dismutase 1 (SOD1) that may have benefit in treating AD and amyotrophic lateral sclerosis (ALS). In particular embodiments, the HuPTM mAb is NI-204, or an antigen binding fragment of NI-204. The amino acid sequences of Fab fragments of this antibody are provided in FIGS. 7A and B. Delivery may be accomplished via gene therapy—e.g., by administering a viral vector or other DNA expression construct encoding an SOD1-binding HuPTM mAb (or an antigen binding fragment and/or a hyperglycosylated derivative or other derivative, thereof) to patients (human subjects) diagnosed with, or having one or more symptoms of AD or ALS to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to SOD1 that can be administered to deliver the HuPTM mAb or antigen binding fragment in a patient. The transgene is a nucleic acid comprising the nucleotide sequences encoding an antigen binding fragment of an antibody that binds to SOD1, such as NI-204, or variants there of as detailed herein. The transgene may also encode an anti-SOD1 antigen binding fragment that contains additional glycosylation sites (e.g., see Courtois et al., 2016, mAbs 8: 99-112 which is incorporated by reference herein in its entirety).

In certain embodiments, the anti-SOD1 antigen-binding fragment transgene comprises the nucleotide sequences encoding the heavy and light chains of the Fab portion of NI-204 (10D12) (having amino acid sequences of SEQ ID NOs. 21 and 22, respectively, see Table 5 and FIG. 7A). The nucleotide sequences may be codon optimized for expression in human cells. Nucleotide sequences may, for example, comprise the nucleotide sequences of SEQ ID NO: 91 (encoding the NI-202 (10D12) heavy chain Fab portion) and SEQ ID NO: 92 (encoding the NI-202 (10D12) light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain and C_{H}1 and C_{L} sequences, the transgenes may comprise, at the C-terminus of the heavy chain C_{H}1 sequence, all or a portion of the hinge region. In specific embodiments, the anti-SOD1-antigen binding domain has a heavy chain Fab fragment of SEQ ID NO: 21 with additional hinge region sequence starting after the C-terminal valine (V), contains all or a portion of the amino acid sequence EPKSCDKTHTCPPCPAPEAAGG (SEQ ID NO: 210), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPEAAGGPSVFL (SEQ ID NO: 212) or EPKSCDKTHLCPPCPAPEAAGGPSVFL (SEQ ID NO: 213) as set forth in FIG. 3. These hinge regions may be encoded by nucleotide sequences at the 3′ end of SEQ ID NO: 91 by the hinge region encoding sequences set forth in Table 6 (SEQ ID NO: 91). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO; 295, or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-SOD1 antigen-binding fragment transgene encodes an SOD1 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 22. In certain embodiments, the anti-SOD1 antigen-binding fragment transgene encodes an SOD1 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 21. In certain embodiments, the anti-SOD1 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 22 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 21. In specific embodiments, the SOD1 antigen binding fragment comprises a heavy chain comprising an amino acid sequence of SEQ ID NO: 21 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 7A) or are substitutions with an amino acid present at that position in the heavy chain of one or more of the other therapeutic antibodies, for example, as identified by the alignment in FIG. 20A. In specific embodiments, the SOD1 antigen binding fragment comprises a light chain comprising an amino acid sequence of SEQ ID NO: 22 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 7A) or are substitutions with an amino acid present at that position in the light chain of one or more of the other therapeutic antibodies, for example, as identified by the alignment in FIG. 20B.

In certain embodiments, the anti-SOD1 antigen-binding fragment transgene encodes a hyperglycosylated NI-202 (10D12) Fab, comprising a heavy chain and a light chain of SEQ ID NOs: 21 and 22, respectively, with one or more of the following mutations: L121N (heavy chain), Q159N or Q159S (light chain), and/or E194N (light chain) (see FIG. 20A (heavy chain) and B (light chain)).

In certain embodiments, the anti-SOD1 antigen-binding fragment transgene encodes an antigen-binding fragment and comprises the nucleotide sequences encoding the six NI-202 (10D12) CDRs which are underlined in the heavy and light chain variable domain sequences of FIG. 7A which are spaced between framework regions, generally human framework regions, and associated with constant domains depending upon the form of the antigen-binding molecule, as is known in the art to form the heavy and/or light chain variable domain of an anti-SOD1 antibody or antigen-binding fragment thereof.

In certain embodiments, the anti-SOD1 antigen-binding fragment transgene comprises the nucleotide sequences encoding the heavy and light chains of the Fab portion of NI-204 (12G7) (having amino acid sequences of SEQ ID NOs. 23 and 24, respectively, see Table 5 and FIG. 7B). The nucleotide sequences may be codon optimized for expression in human cells. Nucleotide sequences may, for example, comprise the nucleotide sequences of SEQ ID NO: 93 (encoding the NI-202 (12G7) heavy chain Fab portion) and SEQ ID NO: 94 (encoding the NI-202 (12G7) light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-SOD1-antigen binding domain has a heavy chain Fab fragment of SEQ ID NO: 23 with additional hinge region sequence starting after the C-terminal valine (V), contains all or a portion of the amino acid sequence EPKSCDKTHTCPPCPAPEAAGG (SEQ ID NO: 210), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPEAAGGPSVFL (SEQ ID NO: 212) or EPKSCDKTHLCPPCPAPEAAGGPSVFL (SEQ ID NO: 213) as set forth in FIG. 7B. These hinge regions may be encoded by nucleotide sequences at the 3′ end of SEQ ID NO: 93 by the hinge region encoding sequences set forth in Table 6 (SEQ ID NO: 93). In another embodiment, the transgenes comprise the amino acid sequences encoding the full length (or substantially full length) heavy and light chains of the antibody, comprising the Fc domain at the C terminus of the heavy chain, e.g., an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-SOD1 antigen-binding fragment transgene encodes an SOD1 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 24. In certain embodiments, the anti-SOD1 antigen-binding fragment transgene encodes an SOD1 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 23. In certain embodiments, the anti-SOD1 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 24 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 23. In specific embodiments, the SOD1 antigen binding fragment comprises a heavy chain comprising an amino acid sequence of SEQ ID NO: 23 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 7B) or are substitutions with an amino acid present at that position in the heavy chain of one or more of the other therapeutic antibodies, for example, as identified by the alignment in FIG. 20A. In specific embodiments, the SOD1 antigen binding fragment comprises a light chain comprising an amino acid sequence of SEQ ID NO: 24 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 7B) or are substitutions with an amino acid present at that position in the light chain of one or more of the other therapeutic antibodies, for example, as identified by the alignment in FIG. 20B.

In certain embodiments, the anti-SOD1 antigen-binding fragment transgene encodes a hyperglycosylated NI-202 (12G7) Fab, comprising a heavy chain and a light chain of SEQ ID NOs: 23 and 24, respectively, with one or more of the following mutations: L118N (heavy chain) and/or Q196N (light chain) (see FIGS. 20A (heavy chain) and 20B (light chain)).

In certain embodiments, the anti-SOD1 antigen-binding fragment transgene encodes an antigen-binding fragment and comprises the nucleotide sequences encoding the six NI-202 (12G7) CDRs which are underlined in the heavy and light chain variable domain sequences of FIG. 7B which are spaced between framework regions, generally human framework regions, and associated with constant domains depending upon the form of the antigen-binding molecule, as is known in the art to form the heavy and/or light chain variable domain of an anti-SOD1 antibody or antigen-binding fragment thereof.

Gene Therapy Methods

Provided are methods of treating human subjects for AD or ALS by administration of a viral vector containing a transgene encoding an anti-SOD1 antibody, or antigen binding fragment thereof. The antibody may be NI-202 and is, e.g., a Fab fragment thereof, or other antigen-binding fragment thereof. In certain embodiments, the patient has been diagnosed with and/or has symptoms associated with prodromal AD, e.g., a mild cognitive impairment associated with early AD or even pre-AD, or ALS.

Recombinant vectors used for delivering the transgene are described in Section 5.4.1. Such vectors should have a tropism for human CNS cells and can include non-replicating rAAV, particularly those bearing an AAV9, AAVrh10, AAVrh20, AAVrh39, or AAVcy5 capsid. The recombinant vectors can be administered in any manner such that the recombinant vector enters the CNS, e.g., by introducing the recombinant vector into the cerebral spinal fluid (CSF). See Section 5.5.1 for details regarding the methods of treatment.

Subjects to whom such gene therapy is administered can be those responsive to anti-SOD1 therapy. In certain embodiments, the methods encompass treating patients who have been diagnosed with AD or ALS, or have one or more symptoms associated therewith, and identified as responsive to treatment with an anti-SOD1 antibody or considered a good candidate for therapy with an anti-SOD1 antibody. In specific embodiments, the patients have previously been treated with NI-202 and have been found to be responsive to NI-202. To determine responsiveness, the anti-SOD1 antibody or antigen-binding fragment transgene product (e.g., produced in human cell culture, bioreactors, etc.) may be administered directly to the subject.

Human Post Translationally Modified Antibodies

The production of the anti-SOD1 HuPTM mAb or HuPTM Fab, should result in a “biobetter” molecule for the treatment of AD or ALS accomplished via gene therapy—e.g., by administering a viral vector or other DNA expression construct encoding the anti-SOD1 HuPTM Fab, intrathecally, particularly intracisternal or lumbar administration, or intravenous administration to human subjects (patients) diagnosed with or having one or more symptoms of AD or ALS, to create a permanent depot in the CNS that continuously supplies the fully-human post-translationally modified, e.g., human-glycosylated, sulfated transgene product produced by transduced CNS cells.

The cDNA construct for the anti-SOD1 HuPTMmAb or anti-SOD1 HuPTM Fab should include a signal peptide that ensures proper co- and post-translational processing (glycosylation and protein sulfation) by the transduced CNS cells. For example, the signal sequence may be MYRMQLLLLIALSLALVTNS (SEQ ID NO: 146).

As an alternative, or an additional treatment to gene therapy, the anti-SOD1 HuPTM mAb or HuPTM Fab can be produced in human cell lines by recombinant DNA technology, and administered to patients diagnosed with AD or ALS, or for whom therapy for AD or ALS is considered appropriate.

In specific embodiments, the anti-SOD1 HuPTM mAb or antigen-binding fragment thereof has heavy and light chains with the amino acid sequences of the heavy and light chain Fab portions of NI-202 (10D12) as set forth in FIG. 7A (with glutamine (Q) glycosylation sites; asparaginal (N) glycosylation sites, non-consensus asparaginal (N) glycosylation sites; and tyrosine-O-sulfation sites (Y) are as indicated in the legend has glycosylation, particularly a 2,6-sialylation, at one or more of the amino acid positions N110 and/or N168 of the heavy chain (SEQ ID NO: 21) or Q99, N157 and/or N209 of the light chain (SEQ ID NO: 22). Alternatively or in addition to, the HuPTM mAb or antigen binding-fragment thereof with the heavy and light chain variable domain sequences of NI-204 has a sulfation group at Y94 of the heavy chain (SEQ ID NO: 21) or Y85 and/or Y86 of the light chain (SEQ ID NO: 22). In other embodiments, the anti-SOD1 HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In specific embodiments, the anti-SOD1 HuPTM mAb or antigen-binding fragment thereof has heavy and light chains with the amino acid sequences of the heavy and light chain Fab portions of NI-202 (12G7) as set forth in FIG. 7B (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N110 and/or N168 of the heavy chain (SEQ ID NO: 23) or Q99, N157 and/or N209 of the light chain (SEQ ID NO: 24). Alternatively or in addition to, the HuPTM mAb or antigen binding-fragment thereof with the heavy and light chain variable domain sequences of NI-204 has a sulfation group at Y94 of the heavy chain (SEQ ID NO: 23) or Y85 and/or Y86 of the light chain (SEQ ID NO: 24). In other embodiments, the anti-SOD1 HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% 2,6 sialylated and/or sulfated and may be at least 5%, 10% or even 50% or 100% glycosylated 2,6 sialylation and/or sulfated. The goal of gene therapy treatment provided herein is to slow or arrest the progression of AD or ALS. In the case of AD, efficacy may be monitored by measuring a reduction in plaque formation and/or an improvement in cognitive function or a reduction in the decline in cognitive function. In the case of ALS, efficacy may be monitored by measuring an improvement in speech and/or a reduction of clumsiness, abnormal limb fatigue, and/or muscle cramps and twitches.

Combinations of delivery of the anti-SOD1 HuPTM mAb or antigen-binding fragment thereof, to the CNS accompanied by delivery of other available treatments are encompassed by the methods provided herein. The additional treatments may be administered before, concurrently or subsequent to the gene therapy treatment. Available treatments for AD that could be combined with the gene therapy provided herein include but are not limited to ARICEPT® (donepezil), RAZADYNE® (galantamine), NAMENDA® (rivastigmine), and NAMZARIC® (donepezil and memantine), to name a few, and administration with anti-SOD1 agents, including but not limited to NI-204. Available treatments for ALS that could be combined with the gene therapy provided herein include but are not limited to RILUTEK® (riluzole), RADICAVA® (edaravone), TIGLUTIK® (riluzole), and NUDEXTRA® (dextromethorphan HBr and quinidine sulfate), to name a few, and administration with anti-SOD1 agents, including but not limited to NI-204.

### 5.3.7. Anti-CGRPR HuPTM Constructs and Formulations for Migraines and Cluster Headaches

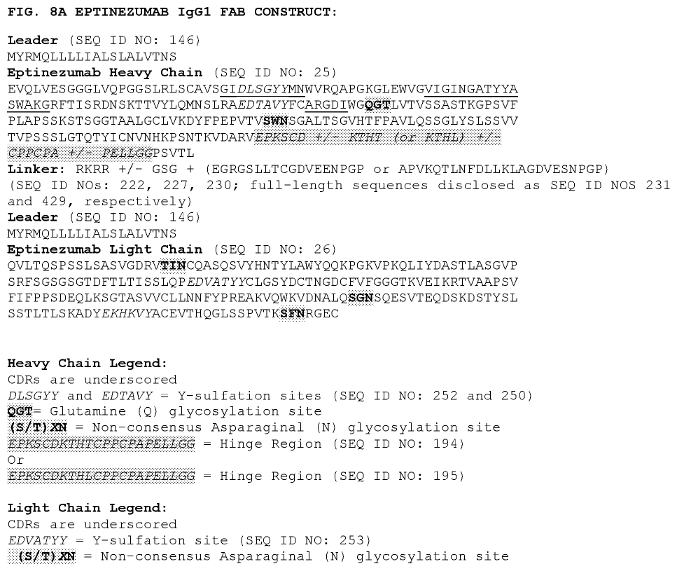

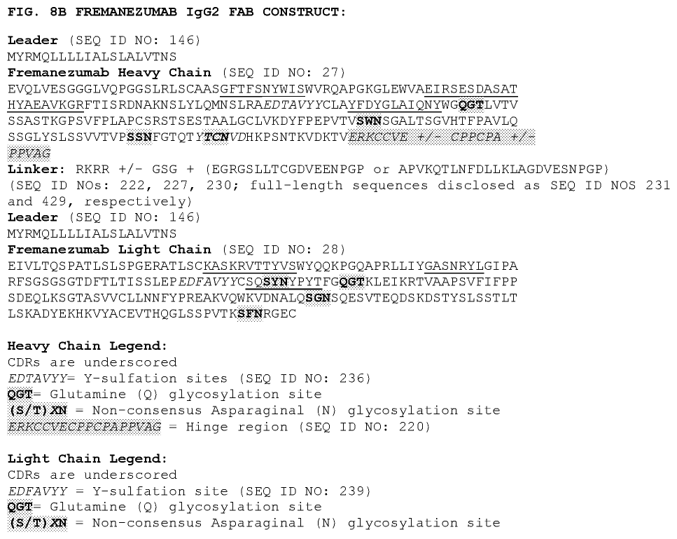

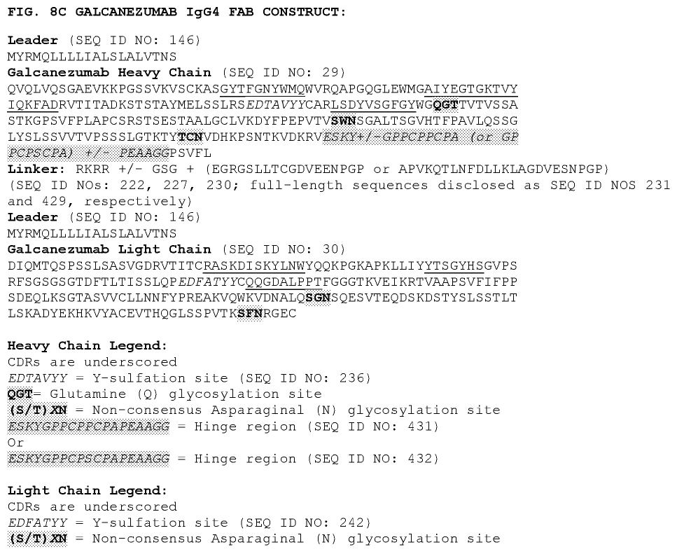

Compositions and methods are described for the delivery of HuPTM mAbs and antigen-binding fragments thereof, such as HuPTM Fabs, that bind to calcitonin gene-related peptide receptor (CGRPR) that may have benefit in treating migraines and cluster headaches (referred to collectively as headache disorders). In certain embodiments, the HuPTM mAb is eptinezumab, fremanezumab, galcanezumab or an antigen binding fragment of one of the foregoing. The amino acid sequences of Fab fragments of these antibodies are provided in FIGS. 8A-C. Delivery may be accomplished via gene therapy—e.g., by administering a viral vector or other DNA expression construct encoding an CGRPR-binding HuPTM mAb (or an antigen binding fragment and/or a hyperglycosylated derivative or other derivative, thereof) to patients (human subjects) diagnosed with, or having one or more symptoms of, migraines and cluster headaches, to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to CGRPR that can be administered to deliver the HuPTM mAb or antigen binding fragment in a patient. The transgene is a nucleic acid comprising the nucleotide sequences encoding an antigen binding fragment of an antibody that binds to CGRPR, such as eptinezumab, fremanezumab, galcanezumab or variants thereof as detailed herein or in accordance with the details herein. The transgene may also encode anti-CGRPR antigen binding fragment that contains additional glycosylation sites (e.g., see Courtois et al., 2016, mAbs 8: 99-112 which is incorporated by reference herein in its entirety).

In certain embodiments, the anti-CGRPR antigen-binding fragment transgene comprises the nucleotide sequences encoding the heavy and light chains of the Fab portion of eptinezumab (having amino acid sequences of SEQ ID NOs. 25 and 26, respectively, see Table 5 and FIG. 8A). The nucleotide sequences may be codon optimized for expression in human cells. Nucleotide sequences may, for example, comprise the nucleotide sequences of SEQ ID NO: 95 (encoding the eptinezumab heavy chain Fab portion) and SEQ ID NO: 96 (encoding the eptinezumab light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-CGRPR-antigen binding domain has a heavy chain Fab fragment of SEQ ID NO: 25 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 8A. These hinge regions may be encoded by nucleotide sequences at the 3′ end of SEQ ID NO: 95 by the hinge region encoding sequences set forth in Table 6 (SEQ ID NO: 95). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 296 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-CGRPR antigen-binding fragment transgene encodes a CGRPR antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 26. In certain embodiments, the anti-CGRPR antigen-binding fragment transgene encodes a CGRPR antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 25. In certain embodiments, the anti-CGRPR antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 26 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 25. In specific embodiments, the CGRPR antigen-binding fragment comprises a heavy chain comprising an amino acid sequence of SEQ ID NO: 25 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 8A) or are substitutions with an amino acid present at that position in the heavy chain of one or more of the other therapeutic antibodies, for example, as identified by the alignment in FIG. 20A. In specific embodiments, the CGRPR antigen-binding fragment comprises a light chain comprising an amino acid sequence of SEQ ID NO: 26 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 8A) or are substitutions with an amino acid present at that position in the light chain of one or more of the other therapeutic antibodies, for example, as identified by the alignment in FIG. 20B.

In certain embodiments, the anti-CGRPR antigen-binding fragment transgene encodes a hyperglycosylated eptinezumab Fab, comprising a heavy chain and a light chain of SEQ ID NOs: 25 and 26, respectively, with one or more of the following mutations: L106N (heavy chain), Q165N or Q165S (light chain), and/or E200N (light chain) (see FIGS. 20A (heavy chain) and 20B (light chain)).

In certain embodiments, the anti-CGRPR antigen-binding fragment transgene encodes an antigen-binding fragment and comprises the nucleotide sequences encoding the six eptinezumab CDRs which are underlined in the heavy and light chain variable domain sequences of FIG. 8A which are spaced between framework regions, generally human framework regions, and associated with constant domains depending upon the form of the antigen-binding molecule, as is known in the art to form the heavy and/or light chain variable domain of an anti-CGRPR antibody or antigen-binding fragment thereof.

In certain embodiments, the anti-CGRPR antigen-binding fragment transgene comprises the nucleotide sequences encoding the heavy and light chains of the Fab portion of fremanezumab (having amino acid sequences of SEQ ID NOs. 27 and 28, respectively, see Table 5 and FIG. 8B). The nucleotide sequences may be codon optimized for expression in human cells. Nucleotide sequences may, for example, comprise the nucleotide sequences of SEQ ID NO: 97 (encoding the fremanezumab heavy chain Fab portion) and SEQ ID NO: 98 (encoding the fremanezumab light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain and C_{H}1 and C_{L} sequences, the transgenes may comprise, at the C-terminus of the heavy chain C_{H}1 domain sequence, all or a portion of the hinge region. In specific embodiments, the anti-CGRPR-antigen binding domain has a heavy chain Fab fragment of SEQ ID NO: 27 with additional hinge region sequence starting after the C-terminal valine (V), contains all or a portion of the amino acid sequence ERKCCVECPPCPAPPVAG (SEQ ID NO: 220) or ERKCCVECPPCPA (SEQ ID NO: 221) as set forth in FIG. 8B. These hinge regions may be encoded by nucleotide sequences at the 3′ end of SEQ ID NO: 97 by the hinge region encoding sequences set forth in Table 6 (SEQ ID NO: 97). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 297 (Table 7) or an IgG2 Fc domain, such as SEQ ID No. 284 or as depicted in FIG. 23, or a mutant or variant thereof. The Fc domain may be engineered for altered binding to one or more Fc receptors and/or effector function as disclosed in Section 5.1.9, infra.

In certain embodiments, the anti-CGRPR antigen-binding fragment transgene encodes a CGRPR antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 28. In certain embodiments, the anti-CGRPR antigen-binding fragment transgene encodes a CGRPR antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 27. In certain embodiments, the anti-CGRPR antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 28 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 27. In specific embodiments, the CGRPR antigen-binding fragment comprises a heavy chain comprising an amino acid sequence of SEQ ID NO: 27 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 8B) or are substitutions with an amino acid present at that position in the heavy chain of one or more of the other therapeutic antibodies, for example, as identified by the alignment in FIG. 20A. In specific embodiments, the CGRPR antigen-binding fragment comprises a light chain comprising an amino acid sequence of SEQ ID NO: 28 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 8B) or are substitutions with an amino acid present at that position in the light chain of one or more of the other therapeutic antibodies, for example, as identified by the alignment in FIG. 20B.

In certain embodiments, the anti-CGRPR antigen-binding fragment transgene encodes a hyperglycosylated fremanezumab Fab, comprising a heavy chain and a light chain of SEQ ID NOs: 27 and 28, respectively, with one or more of the following mutations: L117N (heavy chain), Q160N or Q160S (light chain), and/or E195N (light chain) (see FIGS. 20A (heavy chain) and 20B (light chain)).

In certain embodiments, the anti-CGRPR antigen-binding fragment transgene encodes an antigen-binding fragment and comprises the nucleotide sequences encoding the six fremanezumab CDRs which are underlined in the heavy and light chain variable domain sequences of FIG. 8B which are spaced between framework regions, generally human framework regions, and associated with constant domains depending upon the form of the antigen-binding molecule, as is known in the art to form the heavy and/or light chain variable domain of an anti-CGRPR antibody or antigen-binding fragment thereof.

In certain embodiments, the anti-CGRPR antigen-binding fragment transgene comprises the nucleotide sequences encoding the heavy and light chains of the Fab portion of galcanezumab (having amino acid sequences of SEQ ID NOs. 29 and 30, respectively, see Table 5 and FIG. 8C). The nucleotide sequences may be codon optimized for expression in human cells. Nucleotide sequences may, for example, comprise the nucleotide sequences of SEQ ID NO: 99 (encoding the galcanezumab heavy chain Fab portion) and SEQ ID NO: 100 (encoding the galcanezumab light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-CGRPR-antigen binding domain has a heavy chain Fab domain of SEQ ID NO: 29 with additional hinge region sequence starting after the C-terminal valine (V), contains all or a portion of the amino acid sequence contains all or a portion of the amino acid sequence ESKYGPPCPPCPAPEAAGG (SEQ ID NO: 431) or ESKYGPPCPSCPAPEAAGG (SEQ ID NO: 432) as set forth in FIG. 8C. These hinge regions may be encoded by nucleotide sequences at the 3′ end of SEQ ID NO: 99 by the hinge region encoding sequences set forth in Table 6 (SEQ ID NO: 99). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 298 (Table 7) or an IgG4 Fc domain, such as SEQ ID No. 285 or as depicted in FIG. 23, or a mutant or variant thereof. The Fc domain may be engineered for altered binding to one or more Fc receptors and/or effector function as disclosed in Section 5.1.9, infra.

In certain embodiments, the anti-CGRPR antigen-binding fragment transgene encodes a CGRPR antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 30. In certain embodiments, the anti-CGRPR antigen-binding fragment transgene encodes a CGRPR antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 29. In certain embodiments, the anti-CGRPR antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 30 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 29. In specific embodiments, the CGRPR antigen-binding fragment comprises a heavy chain comprising an amino acid sequence of SEQ ID NO: 29 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 8C) or are substitutions with an amino acid present at that position in the heavy chain of one or more of the other therapeutic antibodies, for example, as identified by the alignment in FIG. 20A. In specific embodiments, the CGRPR antigen-binding fragment comprises a light chain comprising an amino acid sequence of SEQ ID NO: 30 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 8C) or are substitutions with an amino acid present at that position in the light chain of one or more of the other therapeutic antibodies, for example, as identified by the alignment in FIG. 20B.

In certain embodiments, the anti-CGRPR antigen-binding fragment transgene encodes a hyperglycosylated galcanezumab Fab, comprising a heavy chain and a light chain of SEQ ID NOs: 29 and 30, respectively, with one or more of the following mutations: T114N (heavy chain), Q160N or Q160S, and/or E195N (light chain) (see FIGS. 20A (heavy chain) and 20B (light chain)).

In certain embodiments, the anti-CGRPR antigen-binding fragment transgene encodes an antigen-binding fragment and comprises the nucleotide sequences encoding the six galcanezumab CDRs which are underlined in the heavy and light chain variable domain sequences of FIG. 8C which are spaced between framework regions, generally human framework regions, and associated with constant domains depending upon the form of the antigen-binding molecule, as is known in the art to form the heavy and/or light chain variable domain of an anti-CGRPR antibody or antigen-binding fragment thereof.

Gene Therapy Methods

Provided are methods of treating human subjects for migraines and cluster headaches by administration of a viral vector containing a transgene encoding an anti-CGRPR antibody, or antigen binding fragment thereof. The antibody may be eptinezumab, fremanezumab, or galcanezumab and is, e.g., a Fab fragment thereof, or other antigen-binding fragment thereof or is a full length anti-CGRPR antibody with an Fc region. In certain embodiments, the patient has been diagnosed with and/or has symptoms associated with episodic migraines or chronic migraines. In certain embodiments, the patient has been diagnosed with and/or has symptoms associated with episodic cluster headaches or chronic cluster headaches. Recombinant vectors used for delivering the transgenes are described in Section 5.4.1 and shown in FIGS. 8A-C. Such vectors should have a tropism for human CNS cells and can include non-replicating rAAV, particularly those bearing an AAV9, AAVrh10, AAVrh20, AAVrh39, or AAVcy5 capsid. The recombinant vectors can be administered in any manner such that the recombinant vector enters the CNS, e.g., by introducing the recombinant vector into the cerebral spinal fluid (CSF). See Section 5.5.1 for details regarding the methods of treatment.

Subjects to whom such gene therapy is administered can be those responsive to anti-CGRPR therapy. In certain embodiments, the methods encompass treating patients who have been diagnosed with migraines or cluster headaches or have one or more symptoms associated therewith, and identified as responsive to treatment with an anti-CGRPR antibody or considered a good candidate for therapy with an anti-CGRPR antibody. In specific embodiments, the patients have previously been treated with eptinezumab, fremanezumab, or galcanezumab, and have been found to be responsive to one or more of eptinezumab, fremanezumab, and galcanezumab. To determine responsiveness, the anti-CGRPR antibody or antigen-binding fragment transgene product (e.g., produced in human cell culture, bioreactors, etc.) may be administered directly to the subject.

Human Post Translationally Modified Antibodies

The production of the anti-CGRPR HuPTM mAb or HuPTM Fab, should result in a “biobetter” molecule for the treatment of migraines or cluster headaches accomplished via gene therapy—e.g., by administering a viral vector or other DNA expression construct encoding the anti-CGRPR HuPTM Fab, intrathecally, particularly intracisternal or lumbar administration, or intravenous administration to human subjects (patients) diagnosed with or having one or more symptoms of migraines or cluster headaches, to create a permanent depot in the CNS that continuously supplies the fully-human post-translationally modified, e.g., human-glycosylated, sulfated transgene product produced by transduced CNS cells.

The cDNA construct for the anti-CGRPR HuPTM mAb or anti-CGRPR HuPTM Fab should include a signal peptide that ensures proper co- and post-translational processing (glycosylation and protein sulfation) by the transduced CNS cells. For example, the signal sequence may be MYRMQLLLLIALSLALVTNS (SEQ ID NO: 146).

As an alternative, or an additional treatment to gene therapy, the anti-CGRPR HuPTM mAb or HuPTM Fab can be produced in human cell lines by recombinant DNA technology, and administered to patients diagnosed with migraines or cluster headaches, or for whom therapy for migraines or cluster headaches is considered appropriate.

In specific embodiments, the anti-CGRPR HuPTM mAb or antigen-binding fragment thereof has heavy and light chains with the amino acid sequences of the heavy and light chain Fab portions of eptinezumab as set forth in FIG. 8A (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions Q103 and/or N153 of the heavy chain (SEQ ID NO: 25) or N21, N163, and/or N215 of the light chain (SEQ ID NO: 26). Alternatively or in addition to, the HuPTM mAb or antigen binding-fragment thereof with the heavy and light chain variable domain sequences of eptinezumab has a sulfation group at Y32, Y33 and/or Y93 of the heavy chain (SEQ ID NO: 25) and/or Y87 and/or Y88 of the light chain (SEQ ID NO: 26). In other embodiments, the anti-CGRPR HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In specific embodiments, the anti-CGRPR HuPTM mAb or antigen-binding fragment thereof has heavy and light chains with the amino acid sequences of the heavy and light chain Fab portions of fremanezumab as set forth in FIG. 8B (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions Q114, N164, N197 and/or N206 of the heavy chain (SEQ ID NO: 27) or N93, Q100, N158, and/or N210 of the light chain (SEQ ID NO: 28). Alternatively or in addition to, the HuPTM mAb or antigen binding-fragment thereof with the heavy and light chain variable domain sequences of fremanezumab has a sulfation group at Y96, Y97 and/or Y203 of the heavy chain (SEQ ID NO: 27) or Y86 and/or Y87 of the light chain (SEQ ID NO: 28). In other embodiments, the anti-CGRPR HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In specific embodiments, the anti-CGRPR HuPTM mAb or antigen-binding fragment thereof has heavy and light chains with the amino acid sequences of the heavy and light chain Fab portions of galcanezumab as set forth in FIG. 8C (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions Q111, N161, and/or N203 of the heavy chain (SEQ ID NO: 29) or N158 and/or N210 of the light chain (SEQ ID NO: 30). Alternatively or in addition to, the HuPTM mAb or antigen binding-fragment thereof with the heavy and light chain variable domain sequences of erenumab has a sulfation group at Y32 and/or Y33 and/or Y93 of the heavy chain (SEQ ID NO: 29) and/or Y86 and/or Y87 and/or Y92 of the light chain (SEQ ID NO: 30). In other embodiments, the anti-CGRPR HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% 2,6 sialylated and/or sulfated and may be at least 5%, 10% or even 50% or 100% glycosylated 2,6 sialylation and/or sulfated. The goal of gene therapy treatment provided herein is to prevent or reduce the intensity or frequency of migraines, cluster headaches, or one or more of the symptoms associated therewith, including nausea, light sensitivity, sound sensitivity, red eye, eyelid edema, forehead and facial sweating, tearing (lacrimation), abnormal small size of the pupil (miosis), nasal congestion, runny nose (rhinorrhea), and drooping eyelid (ptosis). Efficacy may be monitored by measuring a reduction in the intensity or frequency of migraines or cluster headaches, or a reduction in the amount of acute migraine-specific medication used over a defined period of time.

Combinations of delivery of the anti-CGRPR HuPTM mAb or antigen-binding fragment thereof, to the CNS accompanied by delivery of other available treatments are encompassed by the methods provided herein. The additional treatments may be administered before, concurrently or subsequent to the gene therapy treatment. Available treatments for cluster headaches or migraines that could be combined with the gene therapy provided herein include but are not limited to triptans, ergotamine derivatives and NSAIDs, to name a few, and administration with anti-CGRPR agents, including but not limited to eptinezumab, fremanezumab, and gal canezumab.

### 5.3.8. Anti-VEGF, Anti-EPOR, Anti-Aβ and Anti-Kallikrein HuPTM Constructs and Formulations for Ocular Disorders

Compositions and methods are described for the delivery of HuPTM mAb and antigen-binding fragments thereof, such as HuPTM Fabs, that bind to vascular endothelial growth factor (VEGF), erythropoietin receptor (EPOR), Aβ peptides derived from the amyloid precursor protein, or kallikrein, respectively, indicated for treating one or more retinal disorders including diabetic retinopathy, myopic choroidal neovascularization (mCNV), macular degeneration (e.g., neovascular (wet) or dry age-related macular degeneration (nAMD)), macular edema (e.g., macular edema following a retinal vein occlusion (RVO) or diabetic macular edema (DME)). In certain embodiments, the HuPTM mAb has the amino acid sequence of sevacizumab, LKA-651, GSK933776, lecanemab, or lanadelumab, or an antigen binding fragment of one of the foregoing. The amino acid sequences of Fab fragments of sevacizumab, LKA-651, solanezumab, GSK933776, lecanemab, and lanadelumab are provided in FIGS. 9A-C, 2A-C, and 19, respectively. Delivery may be accomplished via gene therapy—e.g., by administering a viral vector or other DNA expression construct encoding a VEGF-binding, EPOR-binding, Aβ-binding, or kallikrein-binding HuPTM mAb (or an antigen binding fragment and/or a hyperglycosylated derivative or other derivative, thereof, including an scFv) to patients (human subjects) diagnosed with, or having one or more symptoms of a retinal disorder (e.g. diabetic retinopathy, mCNV, macular degeneration, or macular edema) to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to VEGF, EPOR, Aβ or kallikrein, that can be administered to deliver the HuPTM mAb or antigen binding fragment in a patient. The transgene is a nucleic acid comprising the nucleotide sequences encoding an antigen binding fragment of an antibody that binds to VEGF, EPOR, Aft or kallikrein, such as sevacizumab, LKA-651, solanezumab, lecanemab, GSK933776, or lanadelumab, or variants thereof, as detailed herein. The transgene may also encode an anti-VEGF, anti-EPOR, anti-Aft anti-kallikrein antigen binding fragment that contains additional glycosylation sites (e.g., see Courtois et al.).

In certain embodiments, the anti-VEGF antigen-binding fragment transgene comprises the nucleotide sequences encoding the heavy and light chains of the Fab portion of sevacizumab (having amino acid sequences of SEQ ID NOs. 31 and 32, respectively, see Table 5 and FIG. 9A). The nucleotide sequences may be codon optimized for expression in human cells. Nucleotide sequences may, for example, comprise the nucleotide sequences of SEQ ID NO: 101 (encoding the sevacizumab heavy chain Fab portion) and SEQ ID NO: 102 (encoding the sevacizumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-VEGF antigen binding domain has a heavy chain Fab domain of SEQ ID NO: 31 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200), or EPKSCDKTHLCPPCPAPELLGGPSVFL (SEQ ID NO: 201) as set forth in FIG. 9A. These hinge regions may be encoded by nucleotide sequences at the 3′ end of SEQ ID NO: 101 by the hinge region encoding sequences set forth in Table 6 (SEQ ID NO: 101). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 299 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-VEGF antigen-binding fragment transgene encodes an VEGF antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 32. In certain embodiments, the anti-VEGF antigen-binding fragment transgene encodes an VEGF antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 31. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 32 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 31. In specific embodiments, the VEGF antigen binding fragment comprises a heavy chain comprising an amino acid sequence of SEQ ID NO: 31 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 9A) or are substitutions with an amino acid present at that position in the heavy chain of one or more of the other therapeutic antibodies, for example, as identified by the alignment in FIG. 20A. In specific embodiments, the VEGF antigen binding fragment comprises a light chain comprising an amino acid sequence of SEQ ID NO: 32 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 9A) or are substitutions with an amino acid present at that position in the light chain of one or more of the other therapeutic antibodies, for example, as identified by the alignment in FIG. 20B.

In certain embodiments, the anti-VEGF antigen-binding fragment transgene encodes a hyperglycosylated sevacizumab Fab, comprising a heavy chain and a light chain of SEQ ID NOs: 31 and 32, respectively, with one or more of the following mutations: L117N (heavy chain), Q165N or Q165S (light chain), and/or E200N (light chain) (see FIGS. 20A (heavy chain) and 20B (light chain)).

In certain embodiments, the anti-VEGF antigen-binding fragment transgene encodes an antigen-binding fragment and comprises the nucleotide sequences encoding the six sevacizumab CDRs which are underlined in the heavy and light chain variable domain sequences of FIG. 9A which are spaced between framework regions, generally human framework regions, and associated with constant domains depending upon the form of the antigen-binding molecule, as is known in the art to form the heavy and/or light chain variable domain of an anti-VEGF antibody or antigen-binding fragment thereof.

In certain embodiments, the anti-EPOR antigen-binding fragment transgene comprises the nucleotide sequences encoding the heavy and light chains of the Fab portion of LKA-651 (NVS2) (having amino acid sequences of SEQ ID NOs. 33 and 34, respectively, see Table 5 and FIG. 9B). The nucleotide sequences may be codon optimized for expression in human cells. Nucleotide sequences may, for example, comprise the nucleotide sequences of SEQ ID NO: 103 (encoding the LKA-651 (NVS2) heavy chain Fab portion) and SEQ ID NO: 104 (encoding the LKA-651 (NVS2) light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-EPOR antigen binding domain has a heavy chain variable domain of SEQ ID NO: 33 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200), or EPKSCDKTHLCPPCPAPELLGGPSVFL (SEQ ID NO: 201) as set forth in FIG. 9B. These hinge regions may be encoded by nucleotide sequences at the 3′ end of SEQ ID NO: 103 by the hinge region encoding sequences set forth in Table 6 (SEQ ID NO: 103). In another embodiment, the transgenes comprise the amino acid sequences encoding the full length (or substantially full length) heavy and light chains of the antibody, comprising the Fc domain at the C terminus of the heavy chain, e.g., an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-EPOR antigen-binding fragment transgene encodes an EPOR antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 34. In certain embodiments, the anti-EPOR antigen-binding fragment transgene encodes an EPOR antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 33. In certain embodiments, the anti-EPOR antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 34 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 33. In specific embodiments, the EPOR antigen binding fragment comprises a heavy chain comprising an amino acid sequence of SEQ ID NO: 33 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 9B) or are substitutions with an amino acid present at that position in the heavy chain of one or more of the other therapeutic antibodies, for example, as identified by the alignment in FIG. 20A. In specific embodiments, the EPOR antigen binding fragment comprises a light chain comprising an amino acid sequence of SEQ ID NO: 34 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 9B) or are substitutions with an amino acid present at that position in the light chain of one or more of the other therapeutic antibodies, for example, as identified by the alignment in FIG. 20B.

In certain embodiments, the anti-EPOR antigen-binding fragment transgene encodes a hyperglycosylated LKA-651 (NVS2) Fab, comprising a heavy chain and a light chain of SEQ ID NOs: 33 and 34, respectively, with one or more of the following mutations: L112N (heavy chain) and/or Q195N (light chain) (see FIGS. 20A (heavy chain) and 20B (light chain)).

In certain embodiments, the anti-EPOR antigen-binding fragment transgene encodes an antigen-binding fragment and comprises the nucleotide sequences encoding the six LKA-651 (NVS3) CDRs which are underlined in the heavy and light chain variable domain sequences of FIG. 9C which are spaced between framework regions, generally human framework regions, and associated with constant domains depending upon the form of the antigen-binding molecule, as is known in the art to form the heavy and/or light chain variable domain of an anti-EPOR antibody or antigen-binding fragment thereof.

In certain embodiments, the anti-EPOR antigen-binding fragment transgene comprises the nucleotide sequences encoding the heavy and light chains of the Fab portion of LKA-651 (NVS3) (having amino acid sequences of SEQ ID NOs. 35 and 36, respectively, see Table 5 and FIG. 9C). The nucleotide sequences may be codon optimized for expression in human cells. Nucleotide sequences may, for example, comprise the nucleotide sequences of SEQ ID NO: 105 (encoding the LKA-651 (NVS3) heavy chain Fab portion) and SEQ ID NO: 106 (encoding the LKA-651 (NVS3) light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-EPOR antigen binding domain has a heavy chain Fab domain of SEQ ID NO: 35 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200), or EPKSCDKTHLCPPCPAPELLGGPSVFL (SEQ ID NO: 201) as set forth in FIG. 9C. These hinge regions may be encoded by nucleotide sequences at the 3′ end of SEQ ID NO: 105 by the hinge region encoding sequences set forth in Table 6 (SEQ ID NO: 105). In another embodiment, the transgenes comprise the amino acid sequences encoding the full length (or substantially full length) heavy and light chains of the antibody, comprising the Fc domain at the C terminus of the heavy chain, e.g. an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-EPOR antigen-binding fragment transgene encodes an EPOR antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 36. In certain embodiments, the anti-EPOR antigen-binding fragment transgene encodes an EPOR antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 35. In certain embodiments, the anti-EPOR antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 36 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 35. In specific embodiments, the EPOR antigen binding fragment comprises a heavy chain comprising an amino acid sequence of SEQ ID NO: 35 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 9C) or are substitutions with an amino acid present at that position in the heavy chain of one or more of the other therapeutic antibodies, for example, as identified by the alignment in FIG. 20A. In specific embodiments, the EPOR antigen binding fragment comprises a light chain comprising an amino acid sequence of SEQ ID NO: 36 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 9C) or are substitutions with an amino acid present at that position in the light chain of one or more of the other therapeutic antibodies, for example, as identified by the alignment in FIG. 20B.

In certain embodiments, the anti-EPOR antigen-binding fragment transgene encodes a hyperglycosylated LKA-651 (NVS3) Fab, comprising a heavy chain and a light chain of SEQ ID NOs: 35 and 36, respectively, with one or more of the following mutations: L122N (heavy chain) and/or Q195N (light chain) (see FIGS. 20A (heavy chain) and 20B (light chain)).

In certain embodiments, the anti-EPOR antigen-binding fragment transgene encodes an antigen-binding fragment and comprises the nucleotide sequences encoding the six LKA-651 (NVS3) CDRs which are underlined in the heavy and light chain variable domain sequences of FIG. 9C which are spaced between framework regions, generally human framework regions, and associated with constant domains depending upon the form of the antigen-binding molecule, as is known in the art to form the heavy and/or light chain variable domain of an anti-EPOR antibody or antigen-binding fragment thereof.

In certain embodiments, the anti-An antigen-binding fragment transgene comprises the nucleotide sequences encoding the heavy and light chains of the Fab portion of solanezumab (having amino acid sequences of SEQ ID NOs. 1 and 2, respectively, see Table 5 and FIG. 2A). The nucleotide sequences may be codon optimized for expression in human cells. Nucleotide sequences may, for example, comprise the nucleotide sequences of SEQ ID NO: 71 (encoding the solanezumab heavy chain Fab portion) and SEQ ID NO: 72 (encoding the solanezumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the Fab fragments, including the heavy and light chain variable domain sequences and C_{L} C_{H}1, the transgenes may comprise, at the C-terminus of the heavy chain C_{H}1 sequence, all or a portion of the hinge region. In specific embodiments, the anti-Aβ-antigen binding domain has a heavy chain variable domain and C_{H}1 domain of SEQ ID NO: 1 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 2A. These hinge regions may be encoded by nucleotide sequences at the 3′ end of SEQ ID NO: 71 by the hinge region encoding sequences set forth in Table 6 (SEQ ID NO: 71). In another embodiment, the transgenes comprise the amino acid sequences encoding the full length (or substantially full length) heavy and light chains of the antibody, comprising the Fc domain at the C terminus of the heavy chain, e.g. an IgG1 Fc domain, such as SEQ ID NO: 290 of Table 7, SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-Aβ antigen-binding fragment transgene encodes an Aβ antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-Aβ antigen-binding fragment transgene encodes an Aβ antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-Aβ antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In specific embodiments, the Aβ antigen binding fragment comprises a heavy chain comprising an amino acid sequence of SEQ ID NO: 1 with 1, 2, 3, 4, 5, 6, 7, 8, 9, 10, 11, 12, 13, 14, 15 or more amino acid substitutions, insertions or deletions, and the substitutions, insertions or deletions are made for example, in the framework regions (e.g., those regions outside of the CDRs, which CDRs are underlined in FIG. 2A) or are substitutions with an amino acid present at that position in the heavy chain of one or more of the other therapeutic antibodies, for example, as identified by the alignment in FIG. 20A. In specific embodiments, the Aβ antigen binding fragment comprises a light chain comprising an amino acid sequence of SEQ ID NO: 2 with 1, 2, 3, 4, 5, 6, 7, 8, 9, 10, 11, 12, 13, 14, 15 or more amino acid substitutions, insertions or deletions, and the substitutions, insertions or deletions are made for example, in the framework regions (e.g., those regions outside of the CDRs, which CDRs are underlined in FIG. 2A) or are substitutions with an amino acid present at that position in the light chain of one or more of the other therapeutic antibodies, for example, as identified by the alignment in FIG. 20B.

In certain embodiments, the anti-Aβ antigen-binding fragment transgene encodes a hyperglycosylated solanezumab Fab, comprising a heavy chain and a light chain of SEQ ID NOs: 1 and 2, respectively, with one or more of the following mutations: L107N (heavy chain), Q165N or Q165S (light chain), and/or E200N (light chain) (see FIG. 20A (heavy chain) and B (light chain)).

In certain embodiments, the anti-An antigen-binding fragment transgene encodes an antigen-binding fragment and comprises the nucleotide sequences encoding the six solanezumab CDRs which are underlined in the heavy and light chain variable domain sequences of FIG. 2A which are spaced between framework regions, generally human framework regions, and associated with constant domains depending upon the form of the antigen-binding molecule, as is known in the art to form the heavy and/or light chain variable domain of an anti-An antibody or antigen-binding fragment thereof.

In certain embodiments, the anti-Aβ antigen-binding fragment transgene comprises the nucleotide sequences encoding the heavy and light chains of the Fab portion of GSK933776 (having amino acid sequences of SEQ ID NOs. 3 and 4, respectively, see Table 5 and FIG. 2B). The nucleotide sequences may be codon optimized for expression in human cells. Nucleotide sequences may, for example, comprise the nucleotide sequences of SEQ ID NO: 73 (encoding the GSK933776 heavy chain Fab portion) and SEQ ID NO: 74 (encoding the GSK933776 light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-Aβ-antigen binding domain has a heavy chain Fab domain of SEQ ID NO: 3 with additional hinge region sequence starting after the C-terminal valine (V), contains all or a portion of the amino acid sequence EPKSCDKTHTCPPCPAPELAGA (SEQ ID NO: 202), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELAGAPSVFL (SEQ ID NO: 204) or EPKSCDKTHLCPPCPAPELAGAPSVFL (SEQ ID NO: 205) as set forth in FIG. 2B. These hinge regions may be encoded by nucleotide sequences at the 3′ end of SEQ ID NO: 73 by the hinge region encoding sequences set forth in Table 6 (SEQ ID NO: 73). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 291 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-Aβ antigen-binding fragment transgene encodes an Aβ antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-Aβ antigen-binding fragment transgene encodes an Aβ antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, the anti-Aβ antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In specific embodiments, the Aβ antigen binding fragment comprises a heavy chain comprising an amino acid sequence of SEQ ID NO: 3 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2B) or are substitutions with an amino acid present at that position in the heavy chain of one or more of the other therapeutic antibodies, for example, as identified by the alignment in FIG. 20A. In specific embodiments, the Aβ antigen binding fragment comprises a light chain comprising an amino acid sequence of SEQ ID NO: 4 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2B) or are substitutions with an amino acid present at that position in the light chain of one or more of the other therapeutic antibodies, for example, as identified by the alignment in FIG. 20B.

In certain embodiments, the anti-Aβ antigen-binding fragment transgene encodes a hyperglycosylated GSK933776 Fab, comprising a heavy chain and a light chain of SEQ ID NOs: 3 and 4, respectively, with one or more of the following mutations: L110N (heavy chain), Q165N or Q165S (light chain), and/or E200N (light chain) (see FIGS. 20A (heavy chain) and 20B (light chain)).

In certain embodiments, the anti-Aβ antigen-binding fragment transgene encodes an antigen-binding fragment and comprises the nucleotide sequences encoding the six GSK933776 CDRs which are underlined in the heavy and light chain variable domain sequences of FIG. 2B which are spaced between framework regions, generally human framework regions, and associated with constant domains depending upon the form of the antigen-binding molecule, as is known in the art to form the heavy and/or light chain variable domain of an anti-Aβ antibody or antigen-binding fragment thereof.

The anti-kallikrein constructs and HuPTM mAbs and HuPTM Fabs are described in detail in Section 5.3.18, infra. Provided are transgenes for expression of the anti-kallikrein antibody lanadelumab, sequences of which are provided in Table 8.

Gene Therapy Methods

Provided are methods of treating human subjects for one or more retinal disorders (such as diabetic retinopathy, mCNV, macular degeneration, or macular edema) by administration of a viral vector containing a transgene encoding an anti-VEGF antibody or antigen binding fragment thereof. The antibody may be sevacizumab or a Fab fragment thereof or any antigen binding fragment thereof. In embodiments, the patient has been diagnosed with and/or has symptoms associated with one or more of the various retinal disorders listed above.

Also, provided are methods of treating human subjects for one or more retinal disorders (such as diabetic retinopathy, mCNV, macular degeneration, or macular edema) by administration of a viral vector containing a transgene encoding an anti-EPOR antibody or antigen binding fragment thereof. The antibody may be LKA-651, or a Fab fragment thereof, or other antigen-binding fragment thereof. In embodiments, the patient has been diagnosed with and/or has symptoms associated with one or more of the various retinal disorders listed above.

Further provided are methods of treating human subjects for dry AMD degeneration by administration of a viral vector containing a transgene encoding an anti-Aβ antibody or antigen binding fragment thereof. The antibody or Fab fragment thereof may be solanezumab, lecanemab, or GSK933776. In embodiments, the patient has been diagnosed with and/or has symptoms associated with dry AMD.

Provided are methods of treating human subjects for diabetic retinopathy or diabetic macular edema by administration of a viral vector containing a transgene encoding an anti-kallikrein antibody or antigen binding fragment thereof. The antibody may be lanadelumab, or a Fab fragment thereof, or other antigen-binding fragment thereof. In embodiments, the patient has been diagnosed with and/or has symptoms associated with one or more of the various retinal disorders listed above. In specific embodiments, the transgene is a lanadelumab coding transgene in Table 8.

Recombinant vector used for delivering the transgene are described in Section 5.4.3. Such vectors should have a tropism for human retina-type cells and can include non-replicating rAAV, particularly those bearing an AAV8 capsid. Alternatively, vectors bearing an AAV2.7m8 capsid can be used for ocular indications. The recombinant vectors, such as the ones shown in FIGS. 9A-C, 2A-C and 19, can be administered in any manner such that the recombinant vector enters the retina, e.g., by introducing the recombinant vector into the eye. See Section 5.5.3 for details regarding the methods of treatment.

Subjects to whom such gene therapy is administered can be those responsive to anti-VEGF, anti-EPOR, anti-Aβ or anti-kallikrein antibodies. In certain embodiments, the methods encompass treating patients who have been diagnosed with one or more retinal disorders, or have one or more symptoms associated therewith, and identified as responsive to treatment with an anti-VEGF, anti-EPOR, anti-Aβ or anti-kallikrein antibody or considered a good candidate for therapy with an anti-VEGF, anti-EPOR, anti-Aβ or anti-kallikrein antibody. In specific embodiments, the patients have previously been treated with sevacizumab, LKA-651, solanezumab, lecanemab, GSK933776 or lanadelumab and have been found to be responsive to sevacizumab, LKA-651, solanezumab, lecanemab, GSK933776 or lanadelumab. To determine responsiveness, the anti-VEGF, anti-EPOR, anti-Aβ or anti-kallikrein, or antigen-binding fragment transgene product (e.g., produced in cell culture, bioreactors, etc.) may be administered directly to the subject.

Human Post Translationally Modified Antibodies

The production of the anti-VEGF, anti-EPOR, anti-Aβ or anti-kallikrein HuPTM mAb or HuPTM Fab, should result in a “biobetter” molecule for the treatment of one or more retinal disorders accomplished via gene therapy—e.g., by administering a viral vector or other DNA expression construct encoding the anti-VEGF, anti-EPOR, anti-Aβ, anti-kallikrein HuPTM Fab, subretinally, intravitreally, or suprachoroidally to human subjects (patients) diagnosed with or having one or more symptoms of one or more retinal disorders, to create a permanent depot in the retina that continuously supplies the fully-human post-translationally modified, e.g., human-glycosylated, sulfated transgene product produced by transduced cells of the retina.

As an alternative, or an additional treatment to gene therapy, the anti-VEGF, anti-EPOR, anti-Aβ or anti-kallikrein HuPTM mAb or HuPTM Fab can be produced in human cell lines by recombinant DNA technology and administered to patients diagnosed with a retinal disorder for whom therapy for a retinal disorder is considered appropriate.

In specific embodiments, the anti-VEGF HuPTM mAb or antigen-binding fragment thereof has heavy and light chains with the amino acid sequences of the heavy and light chain Fab portions of sevacizumab as set forth in FIG. 9A (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N32 and/or N164 of the heavy chain (SEQ ID NO: 31) or N22, N163, and/or N215 of the light chain (SEQ ID NO: 32). Alternatively or in addition to, the HuPTM mAb or antigen binding-fragment thereof with the heavy and light chain variable domain sequences of sevacizumab has a sulfation group at Y95 of the heavy chain (SEQ ID NO: 31) and/or Y88 and/or Y89 of the light chain (SEQ ID NO: 32). In other embodiments, the anti-VEGF HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-EPOR HuPTM mAb or antigen-binding fragment thereof has heavy and light chains with the amino acid sequences of the heavy and light chain Fab portions of LKA-651 (NVS2) as set forth in FIG. 9B (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions Q109 and/or N159 of the heavy chain (SEQ ID NO: 33) or N68 and/or N171 of the light chain (SEQ ID NO: 34). Alternatively or in addition to, the HuPTM mAb or antigen binding-fragment thereof with the heavy and light chain variable domain sequences of LKA-651 (NVS2) has a sulfation group at Y94 and/or Y95 of the heavy chain (SEQ ID NO: 33) and/or Y85, Y86, and/or Y96 of the light chain (SEQ ID NO: 34). In other embodiments, the anti-EPOR HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-EPOR HuPTM mAb or antigen-binding fragment thereof has heavy and light chains with the amino acid sequences of the heavy and light chain Fab portions of LK-651 (NVS3) as set forth in FIG. 9C (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N32, N80, and/or N169 of the heavy chain (SEQ ID NO: 35) or N68 and/or N171 of the light chain (SEQ ID NO: 36). Alternatively or in addition to, the HuPTM mAb or antigen binding-fragment thereof with the heavy and light chain variable domain sequences of LKA-651 (NVS3) has a sulfation group at Y97 and/or Y98 of the heavy chain (SEQ ID NO: 35) and/or Y30, Y31, Y85 and/or Y86 of the light chain (SEQ ID NO: 36). In other embodiments, the anti-EPOR HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-Aβ HuPTM mAb or antigen-binding fragment thereof has heavy and light chains with the amino acid sequences of the heavy and light chain Fab portions of solanezumab as set forth in FIG. 2A (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N56, Q104, and/or N154 of the heavy chain (SEQ ID NO:1) or Q105, N163 and/or N215 of the light chain (SEQ ID NO: 2). Alternatively, or in addition to, the HuPTM mAb or antigen binding-fragment thereof with the heavy and light chain variable domain sequences of solanezumab has a sulfation group at Y94, Y95 and/or Y101 of the heavy chain (SEQ ID NO: 1) or Y91 and/or Y92 of the light chain (SEQ ID NO: 2). In other embodiments, the anti-Aβ HuPTM mAb or antigen-binding fragment thereof does not contain any detectable (e.g., as detected by assays known in the art, for example, those described in section 5.2, infra) NeuGc moieties and/or does not contain any detectable (e.g., as detected by assays known in the art, for example, those described in section 5.2, infra) alpha-Gal moieties. In certain embodiments, the anti-Aβ-HuPTM mAb is a full length mAb with an Fc region, e.g., the Fc domain of SEQ ID NO: 283, SEQ ID NO: 284 or SEQ ID NO: 285, or a mutant or variant thereof.

In specific embodiments, the anti-Aβ HuPTM mAb or antigen-binding fragment thereof has heavy and light chains with the amino acid sequences of the heavy and light chain Fab portions of GSK933776 as set forth in FIG. 2B (with glutamine (Q) glycosylation sites; asparaginal (N) glycosylation sites, non-consensus asparaginal (N) glycosylation sites; and tyrosine-O-sulfation sites (Y) are as indicated in the legend) has glycosylation, particularly a 2,6-sialylation, at one or more of the amino acid positions N32, Q107, and/or N157 of the heavy chain (SEQ ID NO: 3) or N163 and/or N215 of the light chain (SEQ ID NO: 4). Alternatively or in addition to, the HuPTM mAb or antigen binding-fragment thereof with the heavy and light chain variable domain sequences of GSK933776 has a sulfation group at Y94 and/or Y95 of the heavy chain (SEQ ID NO: 3) or Y91 and/or Y92 of the light chain (SEQ ID NO: 4). In other embodiments, the anti-Aβ HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of a retinal disorder, and/or to suppress angiogenesis. In the case of retinal disorders, efficacy may be monitored by monitoring vision acuity. For example, efficacy can be monitored by assessing change in vision acuity from baseline. (see, e.g., U.S. Department of Health and Human Services Food and Drug Administration Center for Drug Evaluation and Research, Center for Biologics Evaluation and Research. Guidance for industry: clinical trial endpoints for the approval of cancer drugs and biologics. https://www.fda.gov/downloads/Drugs/Guidances/ucm071590.pdf. Published May 2007. Accessed Oct. 13, 2017; Oncology Endpoints in a Changing Landscape. Manag. Care. 2016; 1(suppl):1-12).

Combinations of delivery of the anti-VEGF, anti-EPOR, or anti-Aβ HuPTM mAb or antigen-binding fragment thereof to the retina accompanied by delivery of other available treatments are encompassed by the methods provided herein. The additional treatments may be administered before, concurrently, or subsequent to the gene therapy treatment. Available treatments for diabetic retinopathy, mCNV, macular degeneration, or macular edema that could be combined with the gene therapy provided herein include but are not limited to laser photocoagulation, photodynamic therapy with verteporfin, aflibercept, and/or intravitreal steroids and administration with anti-VEGF, anti-EPOR, anti-Aβ agents, including but not limited to sevacizumab, LKA-651 (NVS2), LKA-651 (NVS3), solanezumab, lecanemab, or GSK933776.

### 5.3.9. Anti-ALK1, Anti-C5, and Anti-Endoglin HuPTM Constructs and Formulations for Ocular Disorders

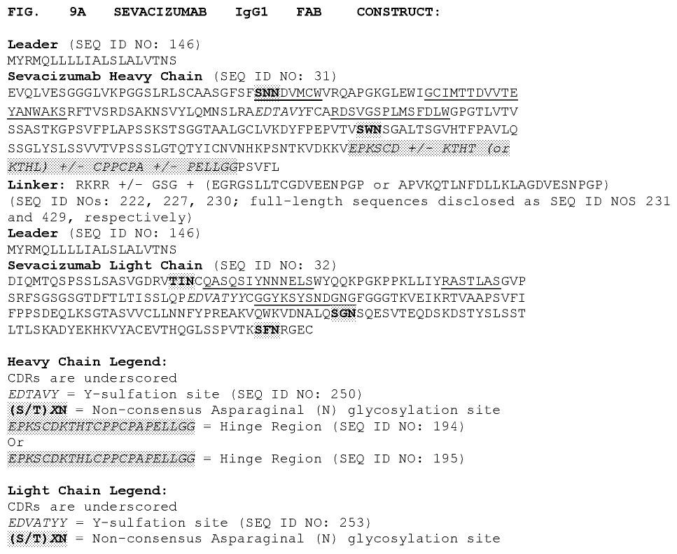

Compositions and methods are described for the delivery of HuPTM mAb and antigen-binding fragments thereof, such as HuPTM Fabs, that bind to activin receptor like kinase 1 (ALK1), complement component 5 (C5), or anti-endoglin (ENG), respectively, indicated for treating one or more ocular disorders including retinal diseases caused by increased neovascularization, for example, nAMD (also known as “wet” AMD), dry AMD, retinal vein occlusion (RVO), diabetic macular edema (DME), diabetic retinopathy (DR), or non-infectious uveitis In certain embodiments, the HuPTM mAb has the amino acid sequence of ascrinvacumab, tesidolumab, ravulizumab, carotuximab, or an antigen binding fragment of one of the foregoing. The amino acid sequences of Fab fragments of ascrinvacumab, tesidolumab, ravulizumab, and carotuximab are provided in FIGS. 10A-D, respectively. Delivery may be accomplished via gene therapy—e.g., by administering a viral vector or other DNA expression construct encoding a ALK1-binding, C5-binding, or ENG-binding HuPTM mAb (or an antigen binding fragment and/or a hyperglycosylated derivative or other derivative, thereof, including an scFv) to patients (human subjects) diagnosed with, or having one or more symptoms of a ocular disorder (e.g. macular degeneration or macular uveitis) to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to ALK1, C5, or ENG that can be administered to deliver the HuPTM mAb or antigen binding fragment in a patient. The transgene is a nucleic acid comprising the nucleotide sequences encoding an antigen binding fragment of an antibody that binds to ALK1, C5, or ENG, such as ascrinvacumab, tesidolumab, ravulizumab, carotuximab, or variants thereof as detailed herein. The transgene may also encode an anti-ALK1, anti-C5, or anti-ENG antigen binding fragment that contains additional glycosylation sites (e.g., see Courtois et al.).

In certain embodiments, the anti-ALK1 antigen-binding fragment transgene comprises the nucleotide sequences encoding the heavy and light chains of the Fab portion of ascrinvacumab (having amino acid sequences of SEQ ID NOs. 37 and 38, respectively, see Table 5 and FIG. 10A). The nucleotide sequences may be codon optimized for expression in human cells. Nucleotide sequences may, for example, comprise the nucleotide sequences of SEQ ID NO: 107 (encoding the ascrinvacumab heavy chain Fab portion) and SEQ ID NO: 108 (encoding the ascrinvacumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-ALK1 antigen binding domain has a heavy chain Fab domain of SEQ ID NO: 37 with additional hinge region sequence starting after the C-terminal valine (V), contains all or a portion of the amino acid sequence contains all or a portion of the amino acid sequence ERKCCVECPPCPAPPVAG (SEQ ID NO: 220) or ERKCCVECPPCPA (SEQ ID NO: 221) as set forth in FIG. 10A. These hinge regions may be encoded by nucleotide sequences at the 3′ end of SEQ ID NO: 107 by the hinge region encoding sequences set forth in Table 6 (SEQ ID NO: 107). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO; 300 (Table 7) or an IgG2 Fc domain, such as SEQ ID No. 284 or as depicted in FIG. 23, or a mutant or variant thereof. The Fc domain may be engineered for altered binding to one or more Fc receptors and/or effector function as disclosed in Section 5.1.9, infra.

In certain embodiments, the anti-ALK1 antigen-binding fragment transgene encodes an ALK1 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 38. In certain embodiments, the anti-ALK1 antigen-binding fragment transgene encodes an ALK1 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 37. In certain embodiments, the anti-ALK1 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 38 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 37. In specific embodiments, the ALK1 antigen binding fragment comprises a heavy chain comprising an amino acid sequence of SEQ ID NO: 37 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 10A) or are substitutions with an amino acid present at that position in the heavy chain of one or more of the other therapeutic antibodies, for example, as identified by the alignment in FIG. 20A. In specific embodiments, the ALK1 antigen binding fragment comprises a light chain comprising an amino acid sequence of SEQ ID NO: 38 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 10A) or are substitutions with an amino acid present at that position in the light chain of one or more of the other therapeutic antibodies, for example, as identified by the alignment in FIG. 20B.

In certain embodiments, the anti-ALK1 antigen-binding fragment transgene encodes a hyperglycosylated ascrinvacumab Fab, comprising a heavy chain and a light chain of SEQ ID NOs: 37 and 38, respectively, with one or more of the following mutations: L113N (heavy chain), Q161N or Q161S (light chain), and/or E196N (light chain) (see FIGS. 20A (heavy chain) and 20B (light chain)).

In certain embodiments, the anti-ALK1 antigen-binding fragment transgene encodes an antigen-binding fragment and comprises the nucleotide sequences encoding the six ascrinvacumab CDRs which are underlined in the heavy and light chain variable domain sequences of FIG. 10A which are spaced between framework regions, generally human framework regions, and associated with constant domains depending upon the form of the antigen-binding molecule, as is known in the art to form the heavy and/or light chain variable domain of an anti-ALK1 antibody or antigen-binding fragment thereof.

In certain embodiments, the anti-C5 antigen-binding fragment transgene comprises the nucleotide sequences encoding the heavy and light chains of the Fab portion of tesidolumab (having amino acid sequences of SEQ ID NOs. 39 and 40, respectively, see Table 5 and FIG. 10B). The nucleotide sequences may be codon optimized for expression in human cells. Nucleotide sequences may, for example, comprise the nucleotide sequences of SEQ ID NO: 109 (encoding the tesidolumab heavy chain Fab portion) and SEQ ID NO: 110 (encoding the tesidolumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-C5 antigen binding domain has a heavy chain Fab domain of SEQ ID NO: 39 with additional hinge region sequence starting after the C-terminal valine (V), contains all or a portion of the amino acid sequence EPKSCDKTHTCPPCPAPEAAGG (SEQ ID NO: 210), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPEAAGGPSVFL (SEQ ID NO: 212), or EPKSCDKTHLCPPCPAPEAAGGPSVFL (SEQ ID NO: 213) as set forth in FIG. 10B. These hinge regions may be encoded by nucleotide sequences at the 3′ end of SEQ ID NO: 109 by the hinge region encoding sequences set forth in Table 6 (SEQ ID NO: 109). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 301 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-C5 antigen-binding fragment transgene encodes an C5 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 40. In certain embodiments, the anti-C5 antigen-binding fragment transgene encodes an C5 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 39. In certain embodiments, the anti-C5 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 40 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 39. In specific embodiments, the C5 antigen binding fragment comprises a heavy chain comprising an amino acid sequence of SEQ ID NO: 39 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 10B) or are substitutions with an amino acid present at that position in the heavy chain of one or more of the other therapeutic antibodies, for example, as identified by the alignment in FIG. 20A. In specific embodiments, the C5 antigen binding fragment comprises a light chain comprising an amino acid sequence of SEQ ID NO: 40 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 10B) or are substitutions with an amino acid present at that position in the light chain of one or more of the other therapeutic antibodies, for example, as identified by the alignment in FIG. 20B.

In certain embodiments, the anti-C5 antigen-binding fragment transgene encodes a hyperglycosylated tesidolumab Fab, comprising a heavy chain and a light chain of SEQ ID NOs: 39 and 40, respectively, with one or more of the following mutations: L111N (heavy chain) and/or Q196N (light chain) (see FIGS. 20A (heavy chain) and 20B (light chain)).

In certain embodiments, the anti-C5 antigen-binding fragment transgene encodes an antigen-binding fragment and comprises the nucleotide sequences encoding the six tesidolumab CDRs which are underlined in the heavy and light chain variable domain sequences of FIG. 10B which are spaced between framework regions, generally human framework regions, and associated with constant domains depending upon the form of the antigen-binding molecule, as is known in the art to form the heavy and/or light chain variable domain of an anti-C5 antibody or antigen-binding fragment thereof.

In certain embodiments, the anti-ENG antigen-binding fragment transgene comprises the nucleotide sequences encoding the heavy and light chains of the Fab portion of carotuximab (having amino acid sequences of SEQ ID NOs. 41 and 42, respectively, see Table 5 and FIG. 10C). The nucleotide sequences may be codon optimized for expression in human cells. Nucleotide sequences may, for example, comprise the nucleotide sequences of SEQ ID NO: 111 (encoding the carotuximab heavy chain Fab portion) and SEQ ID NO: 112 (encoding the carotuximab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-ENG antigen binding domain has a heavy chain Fab domain of SEQ ID NO: 41 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200), or EPKSCDKTHLCPPCPAPELLGGPSVFL (SEQ ID NO: 201) as set forth in FIG. 10C. These hinge regions may be encoded by nucleotide sequences at the 3′ end of SEQ ID NO: 111 by the hinge region encoding sequences set forth in Table 6 (SEQ ID NO: 111). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 302 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-ENG antigen-binding fragment transgene encodes an ENG antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 42. In certain embodiments, the anti-ENG antigen-binding fragment transgene encodes an ENG antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 41. In certain embodiments, the anti-ENG antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 42 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 41. In specific embodiments, the ENG antigen binding fragment comprises a heavy chain comprising an amino acid sequence of SEQ ID NO: 41 with 1, 2, 3, 4, 5, 6, 7, 8, 9, 10, 11, 12, 13, 14, 15 or more amino acid substitutions, insertions or deletions, and the substitutions, insertions or deletions, e.g., are made in the framework regions (e.g., those regions outside of the CDRs, which CDRs are underlined in FIG. 10C) or are substitutions with an amino acid present at that position in the heavy chain of one or more of the other therapeutic antibodies, for example, as identified by the alignment in FIG. 20A. In specific embodiments, the ENG antigen binding fragment comprises a light chain comprising an amino acid sequence of SEQ ID NO: 42 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 10C) or are substitutions with an amino acid present at that position in the light chain of one or more of the other therapeutic antibodies, for example, as identified by the alignment in FIG. 20B.

In certain embodiments, the anti-ENG antigen-binding fragment transgene encodes a hyperglycosylated carotuximab Fab, comprising a heavy chain and a light chain of SEQ ID NOs: 41 and 42, respectively, with one or more of the following mutations: T113N (heavy chain), Q159N or Q159S (light chain), and/or E194N (light chain) (see FIGS. 20A (heavy chain) and 20B (light chain)).

In certain embodiments, the anti-ENG antigen-binding fragment transgene encodes an antigen-binding fragment and comprises the nucleotide sequences encoding the six carotuximab CDRs which are underlined in the heavy and light chain variable domain sequences of FIG. 10C which are spaced between framework regions, generally human framework regions, and associated with constant domains depending upon the form of the antigen-binding molecule, as is known in the art to form the heavy and/or light chain variable domain of an anti-ENG antibody or antigen-binding fragment thereof.

In certain embodiments, the anti-C5 antigen-binding fragment transgene comprises the nucleotide sequences encoding the heavy and light chains of the Fab portion of ravulizumab (having amino acid sequences of SEQ ID NOs. 362 and 363, respectively, see Table 5 and FIG. 10D). The nucleotide sequences may be codon optimized for expression in human cells. Nucleotide sequences may, for example, comprise the nucleotide sequences of SEQ ID NO: 378 (encoding the ravulizumab heavy chain Fab portion) and SEQ ID NO: 379 (encoding the ravulizumab light chain Fab portion) as set forth in Table 6. The heavy and light chain sequences both have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 146) or the one of the sequences found in Table 2 supra.

In addition to the Fab fragments, including the heavy and light chain variable domain sequences and C_{L} C_{H}1, the transgenes may comprise, at the C-terminus of the heavy chain C_{H}1 sequence, all or a portion of the hinge region. In specific embodiments, the anti-C5-antigen binding domain has a heavy chain variable domain and C_{H}1 domain of SEQ ID NO: 362 with additional hinge region sequence starting after the C-terminal valine (V), contains all or a portion of the amino acid sequence ERKCCVECPPCPAPPVAG (SEQ ID NO: 220) or ERKCCVECPPCPA (SEQ ID NO: 221) as set forth in FIG. 10D. These hinge regions may be encoded by nucleotide sequences at the 3′ end of SEQ ID NO: 378 by the hinge region encoding sequences set forth in Table 6 (SEQ ID NO: 378). In another embodiment, the transgenes comprise the amino acid sequences encoding the full length (or substantially full length) heavy and light chains of the antibody, comprising the Fc domain at the C terminus of the heavy chain, e.g. an IgG2 Fc domain, such as SEQ ID NO: 393 of Table 7, SEQ ID No. 284 or as depicted in FIG. 23, or a mutant or variant thereof. The Fc domain may be engineered for altered binding to one or more Fc receptors and/or effector function as disclosed in Section 5.1.9, infra.

In certain embodiments, the anti-C5 antigen-binding fragment transgene encodes an C5 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 363. In certain embodiments, the anti-C5 antigen-binding fragment transgene encodes an C5 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 362. In certain embodiments, the anti-C5 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 363 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 362. In specific embodiments, the C5 antigen binding fragment comprises a heavy chain comprising an amino acid sequence of SEQ ID NO: 362 with 1, 2, 3, 4, 5, 6, 7, 8, 9, 10, 11, 12, 13, 14, 15 or more amino acid substitutions, insertions or deletions, and the substitutions, insertions or deletions are made for example, in the framework regions (e.g., those regions outside of the CDRs, which CDRs are underlined in FIG. 10D) or are substitutions with an amino acid present at that position in the heavy chain of one or more of the other therapeutic antibodies, for example, as identified by the alignment in FIG. 20A. In specific embodiments, the C5 antigen binding fragment comprises a light chain comprising an amino acid sequence of SEQ ID NO: 363 with 1, 2, 3, 4, 5, 6, 7, 8, 9, 10, 11, 12, 13, 14, 15 or more amino acid substitutions, insertions or deletions, and the substitutions, insertions or deletions are made for example, in the framework regions (e.g., those regions outside of the CDRs, which CDRs are underlined in FIG. 10D) or are substitutions with an amino acid present at that position in the light chain of one or more of the other therapeutic antibodies, for example, as identified by the alignment in FIG. 20B.

In certain embodiments, the anti-C5 antigen-binding fragment transgene encodes a hyperglycosylated ravulizumab Fab, comprising a heavy chain and a light chain of SEQ ID NOs: 362 and 363, respectively, with one or more of the following mutations: L117N (heavy chain), Q160N or Q160S (light chain), and/or E195N (light chain) (see FIG. 20A (heavy chain) and B (light chain)).

In certain embodiments, the anti-C5 antigen-binding fragment transgene encodes an antigen-binding fragment and comprises the nucleotide sequences encoding the six ravulizumab CDRs which are underlined in the heavy and light chain variable domain sequences of FIG. 10D which are spaced between framework regions, generally human framework regions, and associated with constant domains depending upon the form of the antigen-binding molecule, as is known in the art to form the heavy and/or light chain variable domain of an anti-C5 antibody or antigen-binding fragment thereof.

Gene Therapy Methods

Provided are methods of treating human subjects for one or more ocular disorders by administration of a viral vector containing a transgene encoding an anti-ENG, anti-C5, or anti-ALK1 antibody or antigen binding fragment thereof. The antibody or Fab fragment thereof may be ascrinvacumab, tesidolumab, ravulizumab, or carotuximab. In embodiments, the patient has been diagnosed with and/or has symptoms associated with one or more of the various ocular disorders listed above. Recombinant vector used for delivering the transgene are described in Section 5.4.3. Such vectors should have a tropism for human retina-type cells and can include non-replicating rAAV, particularly those bearing an AAV8 capsid. Alternatively, vectors bearing an AAV2.7m8 or AAV9 capsid can be used for ocular indications. The recombinant vectors, such as the ones shown in FIGS. 10A-D, can be administered in any manner such that the recombinant vector enters the retina, e.g., by introducing the recombinant vector into the eye. See Section 5.5.3 for details regarding the methods of treatment.

Subjects to whom such gene therapy is administered can be those responsive to anti-ALK1, anti-C5, or anti-ENG. In some embodiments, the methods encompass treating patients who have been diagnosed with one or more retinal disorders or, in the case of an anti-C5 antibody, non-infectious uveitis, or have one or more symptoms associated therewith, and identified as responsive to treatment with an anti-ALK1, anti-C5, or anti-ENG antibody or considered a good candidate for therapy with an anti-ALK1, anti-C5, or anti-ENG antibody. In specific embodiments, the patients have previously been treated with ascrinvacumab, tesidolumab, ravulizumab, or carotuximab and have been found to be responsive to ascrinvacumab, tesidolumab, ravulizumab, or carotuximab. To determine responsiveness, the anti-ALK1, anti-C5, or anti-ENG or antigen-binding fragment transgene product (e.g., produced in cell culture, bioreactors, etc.) may be administered directly to the subject.

Human Post Translationally Modified Antibodies

The production of the anti-ALK1, anti-C5, or anti-ENG HuPTM mAb or HuPTM Fab, should result in a “biobetter” molecule for the treatment of one or more retinal disorders, or in the of case those derived from anti-C5 for the treatment of non-infectious uveitis, accomplished via gene therapy—e.g., by administering a viral vector or other DNA expression construct encoding the anti-ALK1, anti-C5, or anti-ENG HuPTM Fab, subretinally, intravitreally, or suprachoroidally to human subjects (patients) diagnosed with or having one or more symptoms of one or more retinal disorders or non-infectious uveitis, to create a permanent depot in the retina that continuously supplies the fully-human post-translationally modified, e.g., human-glycosylated, sulfated transgene product produced by transduced cells of the retina.

As an alternative, or an additional treatment to gene therapy, the anti-ALK1, anti-C5, or anti-ENG HuPTM mAb or HuPTM Fab can be produced in human cell lines by recombinant DNA technology and administered to patients diagnosed with a retinal or ocular disorder for whom therapy for a retinal or ocular disorder is considered appropriate.

In specific embodiments, the anti-ALK1 HuPTM mAb or antigen-binding fragment thereof has heavy and light chains with the amino acid sequences of the heavy and light chain Fab portions of ascrinvacumab as set forth in FIG. 10A (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N62, N78, Q110, N160, N193, and/or N202 of the heavy chain (SEQ ID NO: 37) or Q101, N159, and/or N211 of the light chain (SEQ ID NO: 38). Alternatively or in addition to, the HuPTM mAb or antigen binding-fragment thereof with the heavy and light chain variable domain sequences of ascrinvacumab has a sulfation group at Y95, Y96, and/or Y199 of the heavy chain (SEQ ID NO: 37) and/or Y87 and/or Y88 of the light chain (SEQ ID NO: 38). In other embodiments, the anti-ALK1 HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-C5 HuPTM mAb or antigen-binding fragment thereof has heavy and light chains with the amino acid sequences of the heavy and light chain Fab portions of tesidolumab as set forth in FIG. 10B (with glutamine (Q) glycosylation sites; asparaginal (N) glycosylation sites, non-consensus asparaginal (N) glycosylation sites; and tyrosine-O-sulfation sites (Y) are as indicated in the legend has a glycosylation, particularly a 2,6-sialylation, at one or more of the amino acid positions N59, Q108, and/or N158 of the heavy chain (SEQ ID NO: 39) or N68, N95, and/or N172 of the light chain (SEQ ID NO: 40). Alternatively or in addition to, the HuPTM mAb or antigen binding-fragment thereof with the heavy and light chain variable domain sequences of tesidolumab has a sulfation group at Y94 and/or Y95 of the heavy chain (SEQ ID NO: 39) and/or Y30, Y85, and/or Y86 of the light chain (SEQ ID NO: 40). In other embodiments, the anti-C5 HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-C5 HuPTM mAb or antigen-binding fragment thereof has heavy and light chains with the amino acid sequences of the heavy and light chain Fab portions of ravulizumab as set forth in FIG. 10D (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N63, N106, Q114, N164, N197, and/or N206 of the heavy chain (SEQ ID NO: 362) or N28, Q100, N158, and/or N210 of the light chain (SEQ ID NO: 363). Alternatively or in addition to, the HuPTM mAb or antigen binding-fragment thereof with the heavy and light chain variable domain sequences of ravulizumab has a sulfation group at Y94 and/or Y95 of the heavy chain (SEQ ID NO: 362) and/or Y86 and/or Y86 of the light chain (SEQ ID NO: 363). In other embodiments, the anti-C5 HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-ENG HuPTM mAb or antigen-binding fragment thereof has heavy and light chains with the amino acid sequences of the heavy and light chain Fab portions of carotuximab as set forth in FIG. 10C (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions Q110 and/or N160 of the heavy chain (SEQ ID NO: 41) or N52, N93, N157, and/or N209 of the light chain (SEQ ID NO: 42). Alternatively or in addition to, the HuPTM mAb or antigen binding-fragment thereof with the heavy and light chain variable domain sequences of carotuximab has a sulfation group at Y95 and/or Y96 of the heavy chain (SEQ ID NO: 41) and/or Y85 and/or Y86 of the light chain (SEQ ID NO: 42). In other embodiments, the anti-ENG HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of an ocular disorder. In the case of retinal disorders, efficacy may be monitored by monitoring vision acuity. For example, efficacy can be monitored by assessing change in vision acuity. In the case of uveitis, efficacy may be monitored by monitoring vision acuity, redness of the eye, sensitivity to light, and/or eye pain. For example, efficacy can be monitored by assessing change in vision acuity, redness of the eye, sensitivity to light, and/or eye pain from baseline.

Combinations of delivery of the anti-ALK1, anti-C5, or anti-ENG HuPTM mAb or antigen-binding fragment thereof to the retina accompanied by delivery of other available treatments are encompassed by the methods provided herein. The additional treatments may be administered before, concurrently, or subsequent to the gene therapy treatment. Available treatments for macular degeneration that could be combined with the gene therapy provided herein include but are not limited to laser photocoagulation, photodynamic therapy with verteporfin, aflibercept, anti-VEGF agents, and/or intravitreal steroids and administration with anti-ALK1, anti-C5, or anti-ENG agents, including but not limited to ascrinvacumab, tesidolumab, ravulizumab, or carotuximab. In the case of uveitis, available treatments for a subject that could be combined with the gene therapy provided herein include but are not limited to, azathioprine, methotrexate, mycophenolate mofetil, cyclosporine, cyclophosphamide, corticosteroids (local and/or systemic), and others and administration with anti-ALK1, anti-C5, or anti-ENG agents, including but not limited to ascrinvacumab, tesidolumab, ravulizumab, or carotuximab.

### 5.3.10. Anti-CC1Q HuPTM Constructs and Formulations for Glaucoma

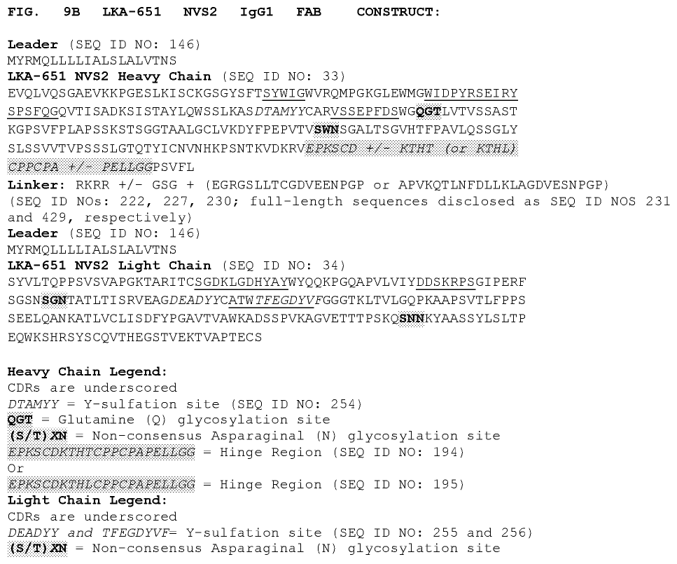

Compositions and methods are described for the delivery of HuPTM mAb and antigen-binding fragments thereof, such as HuPTM Fabs, that bind to complement component 1Q (CC1Q) indicated for treating glaucoma. In certain embodiments, the HuPTM mAb has the amino acid sequence of ANX-007 or an antigen binding fragment of the foregoing. The amino acid sequence of Fab fragments of ANX-007 are provided in FIG. 11. Delivery may be accomplished via gene therapy—e.g., by administering a viral vector or other DNA expression construct encoding a CC1Q-binding HuPTM mAb (or an antigen binding fragment and/or a hyperglycosylated derivative or other derivative, thereof, including an scFv) to patients (human subjects) diagnosed with, or having one or more symptoms of glaucoma to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to CC1Q that can be administered to deliver the HuPTM mAb or antigen binding fragment in a patient. The transgene is a nucleic acid comprising the nucleotide sequences encoding an antigen binding fragment of an antibody that binds to CC1Q, such as ANX-007, or variants thereof as detailed herein. The transgene may also encode an anti-CC1Q antigen binding fragment that contains additional glycosylation sites (e.g., see Courtois et al.).

In certain embodiments, the anti-CC1Q antigen-binding fragment transgene comprises the nucleotide sequences encoding the heavy and light chains of the Fab portion of ANX-007 (having amino acid sequences of SEQ ID NOs. 43 and 44, respectively, see Table 5 and FIG. 11). The nucleotide sequences may be codon optimized for expression in human cells. Nucleotide sequences may, for example, comprise the nucleotide sequences of SEQ ID NO: 113 (encoding the ANX-007 heavy chain Fab portion) and SEQ ID NO: 114 (encoding the ANX-007 light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-CC1Q antigen binding domain has a heavy chain Fab domain of SEQ ID NO: 43 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200), or EPKSCDKTHLCPPCPAPELLGGPSVFL (SEQ ID NO: 201) as set forth in FIG. 11. These hinge regions may be encoded by nucleotide sequences at the 3′ end of SEQ ID NO: 113 by the hinge region encoding sequences set forth in Table 6 (SEQ ID NO: 113). In another embodiment, the transgenes comprise the amino acid sequences encoding the full length (or substantially full length) heavy and light chains of the antibody, comprising the Fc domain at the C terminus of the heavy chain, e.g. an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-CC1Q antigen-binding fragment transgene encodes an CC1Q antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 44. In certain embodiments, the anti-CC1Q antigen-binding fragment transgene encodes an CC1Q antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 43. In certain embodiments, the anti-CC1Q antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 44 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 43. In specific embodiments, the CC1Q antigen binding fragment comprises a heavy chain comprising an amino acid sequence of SEQ ID NO: 43 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 11) or are substitutions with an amino acid present at that position in the heavy chain of one or more of the other therapeutic antibodies, for example, as identified by the alignment in FIG. 20A. In specific embodiments, the CC1Q antigen binding fragment comprises a light chain comprising an amino acid sequence of SEQ ID NO: 44 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 11) or are substitutions with an amino acid present at that position in the light chain of one or more of the other therapeutic antibodies, for example, as identified by the alignment in FIG. 20B.

In certain embodiments, the anti-CC1Q antigen-binding fragment transgene encodes a hyperglycosylated ANX-007 Fab, comprising a heavy chain and a light chain of SEQ ID NOs: 43 and 44, respectively, with one or more of the following mutations: T116N (heavy chain), Q160N or Q160S (light chain), and/or E195N (light chain) (see FIGS. 20A (heavy chain) and 20B (light chain)).

In certain embodiments, the anti-CC1Q antigen-binding fragment transgene encodes an antigen-binding fragment and comprises the nucleotide sequences encoding the six ANX-007 CDRs which are underlined in the heavy and light chain variable domain sequences of FIG. 11 which are spaced between framework regions, generally human framework regions, and associated with constant domains depending upon the form of the antigen-binding molecule, as is known in the art to form the heavy and/or light chain variable domain of an anti-CC1Q antibody or antigen-binding fragment thereof.

Gene Therapy Methods

Provided are methods of treating human subjects for glaucoma by administration of a viral vector containing a transgene encoding an anti-CC1Q antibody or antigen binding fragment thereof. The antibody or Fab fragment thereof may be ANX-007. In embodiments, the patient has been diagnosed with and/or has symptoms associated with one or more of the various retinal disorders listed above.

Recombinant vector used for delivering the transgene are described in Section 5.4.3. Such vectors should have a tropism for human retina-type cells and can include non-replicating rAAV, particularly those bearing an AAV8 capsid. Alternatively, vectors bearing an AAV2.7m8 or AAV9 capsid can be used for ocular indications. The recombinant vectors, such as the ones shown in FIG. 11, can be administered in any manner such that the recombinant vector enters the retina, e.g. by introducing the recombinant vector into the eye. See Section 5.5.3 for details regarding the methods of treatment.

Subjects to whom such gene therapy is administered can be those responsive to anti-CC1Q. In certain embodiments, the methods encompass treating patients who have been diagnosed with glaucoma, or have one or more symptoms associated therewith, and identified as responsive to treatment with an anti-CC1Q antibody or considered a good candidate for therapy with an anti-CC1Q antibody. In specific embodiments, the patients have previously been treated with ANX-007, and have been found to be responsive to ANX-007. To determine responsiveness, the anti-CC1Q or antigen-binding fragment transgene product (e.g., produced in cell culture, bioreactors, etc.) may be administered directly to the subject.

Human Post Translationally Modified Antibodies

The production of the anti-CC1Q HuPTM mAb or HuPTM Fab, should result in a “biobetter” molecule for the treatment of one or more retinal disorders accomplished via gene therapy—e.g., by administering a viral vector or other DNA expression construct encoding the anti-CC1Q HuPTM Fab, subretinally, intravitreally, or suprachoroidally to human subjects (patients) diagnosed with or having one or more symptoms of glaucoma, to create a permanent depot in the retina that continuously supplies the fully-human post-translationally modified, e.g., human-glycosylated, sulfated transgene product produced by transduced cells of the retina.

As an alternative, or an additional treatment to gene therapy, the anti-CC1Q HuPTM mAb or HuPTM Fab can be produced in human cell lines by recombinant DNA technology, and administered to patients diagnosed with glaucoma or for whom therapy for glaucoma is considered appropriate.

In specific embodiments, the anti-CC1Q HuPTM mAb or antigen-binding fragment thereof has heavy and light chains with the amino acid sequences of the heavy and light chain Fab portions of ANX-007 as set forth in FIG. 11 (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N59, Q113, and/or N163 of the heavy chain (SEQ ID NO: 43) or N22, N30, Q100, N158, and/or N210 of the light chain (SEQ ID NO: 44). Alternatively or in addition to, the HuPTM mAb or antigen binding-fragment thereof with the heavy and light chain variable domain sequences of ANX-007 has a sulfation group at Y60, Y94, and/or Y95 of the heavy chain (SEQ ID NO: 43) and/or Y86 and/or Y87 of the light chain (SEQ ID NO: 44). In other embodiments, the anti-CC1Q HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of glaucoma. In the case of glaucoma, efficacy may be monitored by monitoring vision acuity, eye pain, or intraocular pressure (TOP). For example, efficacy can be monitored by assessing change in TOP, vision acuity, and pain from baseline.

Combinations of delivery of the anti-CC1Q HuPTM mAb or antigen-binding fragment thereof to the retina accompanied by delivery of other available treatments are encompassed by the methods provided herein. The additional treatments may be administered before, concurrently, or subsequent to the gene therapy treatment. Available treatments for glaucoma that could be combined with the gene therapy provided herein include but are not limited to prostaglandins (XALATAN®, LUMIGAN®, TRAVATAN Z®, and RESCULA®), alpha-andronergic agonists (IOPIDINE®, ALPHAGAN®, and ALPHAGAN-P®), carbonic anhydrase inhibitors (TRUSOPT®, AZOPT®, DIAMOX®, NEPTAZANE®, and DARANIDE®), parasympathomimetics (PILOCARPINE®, CARBACHOL®, ECHOTHIOPHATE®, DEMACARIUM®), and/or beta-blocker (TIMOPTIC XE®, ISTALOL®, BETOPTIC S®) and administration with anti-CC1Q agents, including but not limited ANX-007.

### 5.3.11. Anti-TNFα HuPTM Constructs and Formulations for Non-Infectious Uveitis

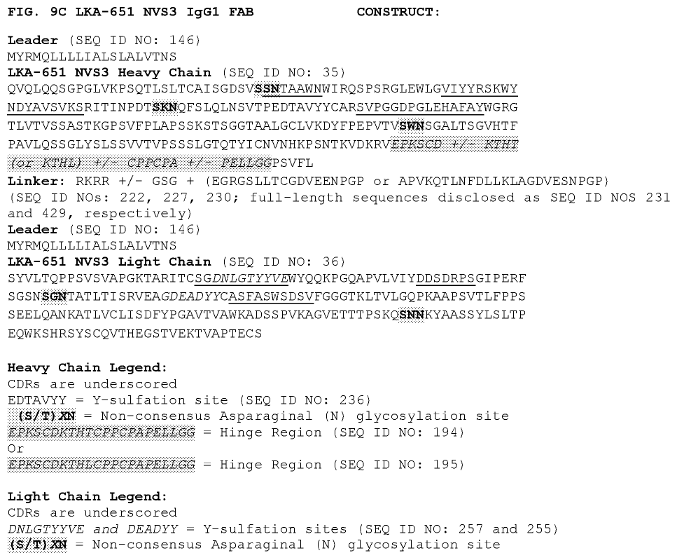

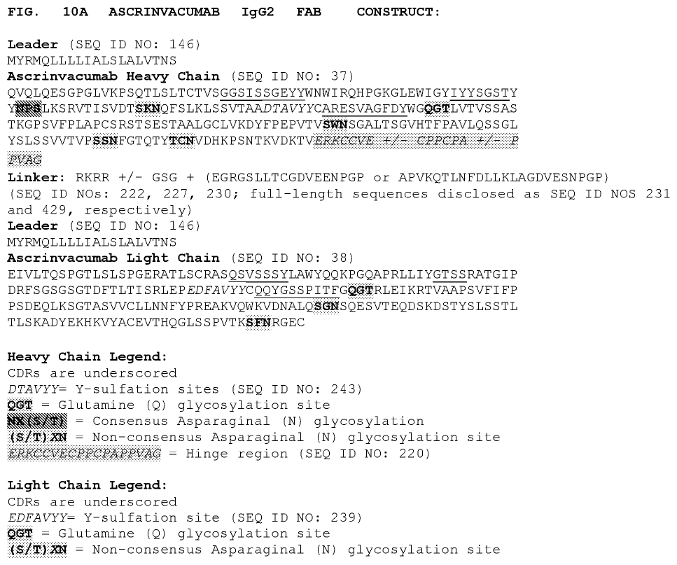

Compositions and methods are described for the delivery of HuPTM mAbs and antigen-binding fragments thereof, such as HuPTM Fabs, that bind to tumor necrosis factor-alpha (TNFα), such as adalimumab (FIG. 12A), infliximab (FIG. 12B), or golimumab (FIG. 12C) and indicated for treating non-infectious uveitis. In certain embodiments, the HuPTM mAb has the amino acid sequence of adalimumab, infliximab, golimumab, or an antigen binding fragment thereof. Amino acid sequences of Fab fragments of the antibody are provided in FIGS. 12A-C. Delivery may be accomplished via gene therapy—e.g., by administering a viral vector or other DNA expression construct encoding an TNFα-binding HuPTM mAb (or an antigen binding fragment and/or a hyperglycosylated derivative or other derivative, thereof) to patients (human subjects) diagnosed with one or more symptoms of non-infectious uveitis to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to TNFα that can be administered to deliver the HuPTM mAb or antigen binding fragment in a patient. The transgene is a nucleic acid comprising the nucleotide sequences encoding an antigen binding fragment of an antibody that binds to TNFα, such as adalimumab, infliximab, golimumab, or variants thereof as detailed herein. The transgene may also encode an anti-TNFα antigen binding fragment that contains additional glycosylation sites (e.g., see Courtois et al.).

In certain embodiments, the anti-TNFα antigen-binding fragment transgene comprises the nucleotide sequences encoding the heavy and light chains of the Fab portion of adalimumab (having amino acid sequences of SEQ ID NOs. 45 and 46, respectively, see Table 5 and FIG. 12A). The nucleotide sequences may be codon optimized for expression in human cells. Nucleotide sequences may, for example, comprise the nucleotide sequences of SEQ ID NO: 115 (encoding the adalimumab heavy chain Fab portion) and SEQ ID NO: 116 (encoding the adalimumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-TNFα-antigen binding domain has a heavy chain Fab domain of SEQ ID NO: 45 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 12A. These hinge regions may be encoded by nucleotide sequences at the 3′ end of SEQ ID NO: 115 by the hinge region encoding sequences set forth in Table 6 (SEQ ID NO: 115). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 303 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-TNFα antigen-binding fragment transgene encodes an TNFα antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 46. In certain embodiments, the anti-TNFα antigen-binding fragment transgene encodes an TNFα antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 45. In certain embodiments, the anti-TNFα antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 46 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 45. In specific embodiments, the TNFα antigen binding fragment comprises a heavy chain comprising an amino acid sequence of SEQ ID NO: 45 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 12A) or are substitutions with an amino acid present at that position in the heavy chain of one or more of the other therapeutic antibodies, for example, as identified by the alignment in FIG. 20A. In specific embodiments, the TNFα antigen binding fragment comprises a light chain comprising an amino acid sequence of SEQ ID NO: 46 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 12A) or are substitutions with an amino acid present at that position in the light chain of one or more of the other therapeutic antibodies, for example, as identified by the alignment in FIG. 20B.

In certain embodiments, the anti-TNFα antigen-binding fragment transgene encodes a hyperglycosylated adalimumab Fab, comprising a heavy chain and a light chain of SEQ ID NOs: 45 and 46, respectively, with one or more of the following mutations: L116N (heavy chain), Q160N or Q160S (light chain), and/or E195N (light chain) (see FIGS. 20A (heavy chain) and 20B (light chain)).

In certain embodiments, the anti-TNFα antigen-binding fragment transgene encodes an antigen-binding fragment and comprises the nucleotide sequences encoding the six adalimumab CDRs which are underlined in the heavy and light chain variable domain sequences of FIG. 12A which are spaced between framework regions, generally human framework regions, and associated with constant domains depending upon the form of the antigen-binding molecule, as is known in the art to form the heavy and/or light chain variable domain of an anti-TNFα antibody or antigen-binding fragment thereof.

In certain embodiments, the anti-TNFα antigen-binding fragment transgene comprises the nucleotide sequences encoding the heavy and light chains of the Fab portion of infliximab (having amino acid sequences of SEQ ID NOs. 47 and 48, respectively, see Table 5 and FIG. 12B). The nucleotide sequences may be codon optimized for expression in human cells. Nucleotide sequences may, for example, comprise the nucleotide sequences of SEQ ID NO: 117 (encoding the infliximab heavy chain Fab portion) and SEQ ID NO: 118 (encoding the infliximab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-TNFα-antigen binding domain has a heavy chain Fab domain of SEQ ID NO: 47 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 12B. These hinge regions may be encoded by nucleotide sequences at the 3′ end of SEQ ID NO: 117 by the hinge region encoding sequences set forth in Table 6 (SEQ ID NO: 117). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 304 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-TNFα antigen-binding fragment transgene encodes an TNFα antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 48. In certain embodiments, the anti-TNFα antigen-binding fragment transgene encodes an TNFα antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 47. In certain embodiments, the anti-TNFα antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 48 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 47. In specific embodiments, the TNFα antigen binding fragment comprises a heavy chain comprising an amino acid sequence of SEQ ID NO: 47 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 12B) or are substitutions with an amino acid present at that position in the heavy chain of one or more of the other therapeutic antibodies, for example, as identified by the alignment in FIG. 20A. In specific embodiments, the TNFα antigen binding fragment comprises a light chain comprising an amino acid sequence of SEQ ID NO: 48 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIG. 12B) or are substitutions with an amino acid present at that position in the light chain of one or more of the other therapeutic antibodies, for example, as identified by the alignment in FIG. 20B.

In certain embodiments, the anti-TNFα antigen-binding fragment transgene encodes a hyperglycosylated infliximab Fab, comprising a heavy chain and a light chain of SEQ ID NOs: 47 and 48, respectively, with one or more of the following mutations: T115N (heavy chain), Q160N or Q160S (light chain), and/or E195N (light chain) (see FIGS. 11A (heavy chain) and 20B (light chain)).

In certain embodiments, the anti-TNFα antigen-binding fragment transgene encodes an antigen-binding fragment and comprises the nucleotide sequences encoding the six infliximab CDRs which are underlined in the heavy and light chain variable domain sequences of FIG. 12B which are spaced between framework regions, generally human framework regions, and associated with constant domains depending upon the form of the antigen-binding molecule, as is known in the art to form the heavy and/or light chain variable domain of an anti-TNFα antibody or antigen-binding fragment thereof.

In certain embodiments, the anti-TNFα antigen-binding fragment transgene comprises the nucleotide sequences encoding the heavy and light chains of the Fab portion of golimumab (having amino acid sequences of SEQ ID NOs. 49 and 50, respectively, see Table 5 and FIG. 12C). The nucleotide sequences may be codon optimized for expression in human cells. Nucleotide sequences may, for example, comprise the nucleotide sequences of SEQ ID NO: 119 (encoding the golimumab heavy chain Fab portion) and SEQ ID NO: 120 (encoding the golimumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-TNFα-antigen binding domain has a heavy chain variable domain of SEQ ID NO: 49 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 12C. These hinge regions may be encoded by nucleotide sequences at the 3′ end of SEQ ID NO: 119 by the hinge region encoding sequences set forth in Table 6 (SEQ ID NO: 119). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO; 305 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-TNFα antigen-binding fragment transgene encodes an TNFα antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 50. In certain embodiments, the anti-TNFα antigen-binding fragment transgene encodes an TNFα antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 49. In certain embodiments, the anti-TNFα antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 50 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 49. In specific embodiments, the TNFα antigen binding fragment comprises a heavy chain comprising an amino acid sequence of SEQ ID NO: 49 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIG. 12C) or are substitutions with an amino acid present at that position in the heavy chain of one or more of the other therapeutic antibodies, for example, as identified by the alignment in FIG. 20A. In specific embodiments, the TNFα antigen binding fragment comprises a light chain comprising an amino acid sequence of SEQ ID NO: 50 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 12C) or are substitutions with an amino acid present at that position in the light chain of one or more of the other therapeutic antibodies, for example, as identified by the alignment in FIG. 20B.

In certain embodiments, the anti-TNFα antigen-binding fragment transgene encodes a hyperglycosylated golimumab Fab, comprising a heavy chain and a light chain of SEQ ID NOs: 49 and 50, respectively, with one or more of the following mutations: T124N (heavy chain), Q164N or Q164S (light chain), and/or E199N (light chain) (see FIGS. 20A (heavy chain) and 20B (light chain)).

In certain embodiments, the anti-TNFα antigen-binding fragment transgene encodes an antigen-binding fragment and comprises the nucleotide sequences encoding the six golimumab CDRs which are underlined in the heavy and light chain variable domain sequences of FIG. 12C which are spaced between framework regions, generally human framework regions, and associated with constant domains depending upon the form of the antigen-binding molecule, as is known in the art to form the heavy and/or light chain variable domain of an anti-TNFα antibody or antigen-binding fragment thereof.

Gene Therapy Methods

Provided are methods of treating human subjects for non-infectious uveitis by administration of a viral vector containing a transgene encoding an anti-TNFα antibody, or antigen binding fragment thereof. The antibody may be adalimumab, infliximab, or golimumab, and is, e.g., a full length or substantially full length antibody or Fab fragment thereof, or other antigen-binding fragment thereof. In embodiments, the patient has been diagnosed with and/or has symptom(s) associated with non-infectious uveitis. Recombinant vector used for delivering the transgene are described in Section 5.4.3. Such vectors should have a tropism for human retina-type cells and can include non-replicating rAAV, particularly those bearing an AAV8 capsid. Alternatively, vectors bearing an AAV2.7m8 or AAV9 capsid can be used for ocular indications. The recombinant vectors, such as the ones shown in FIG. 12A-C, can be administered in any manner such that the recombinant vector enters the retina, e.g. by introducing the recombinant vector into the eye. See Section 5.5.3 for details regarding the methods of treatment.

Subjects to whom such gene therapy is administered can be those responsive to anti-TNFα therapy. In certain embodiments, the methods encompass treating patients who have been diagnosed with non-infectious uveitis, or have one or more symptoms associated therewith, and identified as responsive to treatment with an anti-TNFα antibody or considered a good candidate for therapy with an anti-TNFα antibody. In specific embodiments, the patients have previously been treated with adalimumab, infliximab, or golimumab, and have been found to be responsive to adalimumab, infliximab, or golimumab. In other embodiments, the patients have been previously treated with an anti-TNF-alpha antibody or fusion protein such as etanercept, certolizumab, or other anti-TNF-alpha agent. To determine responsiveness, the anti-TNFα antibody or antigen-binding fragment transgene product (e.g., produced in cell culture, bioreactors, etc.) may be administered directly to the subject.

Human Post Translationally Modified Antibodies

The production of the anti-TNFα HuPTM mAb or HuPTM Fab, should result in a “biobetter” molecule for the treatment of non-infectious uveitis accomplished via gene therapy—e.g., by administering a viral vector or other DNA expression construct encoding the anti-TNFα HuPTM Fab, intravenously to human subjects (patients) diagnosed with or having one or more symptoms of non-infectious uveitis, to create a permanent depot in the retina that continuously supplies the fully-human post-translationally modified, e.g., human-glycosylated, sulfated transgene product produced by transduced liver or muscle cells.

In specific embodiments, the anti-TNFα HuPTM mAb or antigen-binding fragment thereof has heavy and light chains with the amino acid sequences of the heavy and light chain Fab portions of adalimumab as set forth in FIG. 12A (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N54, Q113, and/or N163 of the heavy chain (SEQ ID NO:45) or Q100, N158, and/or N210 of the light chain (SEQ ID NO:46). Alternatively or in addition to, the HuPTM mAb or antigen binding-fragment thereof with the heavy and light chain variable domain sequences of adalimumab has a sulfation group at Y32, Y94 and/or Y95 of the heavy chain (SEQ ID NO:45) and/or Y86 and/or Y87 of the light chain (SEQ ID NO:46). In other embodiments, the anti-TNFα HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-TNFα HuPTM mAb or antigen-binding fragment thereof has heavy and light chains with the amino acid sequences of the heavy and light chain Fab portions of infliximab as set forth in FIG. 12B (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N57, N101, Q112 and/or N162 of the heavy chain (SEQ ID NO:47) or N41, N76, N158 and/or N210 of the light chain (SEQ ID NO:48). Alternatively or in addition to, the HuPTM mAb or antigen binding-fragment thereof with the heavy and light chain variable domain sequences of infliximab has a sulfation group at Y96 and/or Y97 of the heavy chain (SEQ ID NO:47) and/or Y86 and/or Y87 of the light chain (SEQ ID NO:48). In other embodiments, the anti-TNFα HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-TNFα HuPTM mAb or antigen-binding fragment thereof has heavy and light chains with the amino acid sequences of the heavy and light chain Fab portions of golimumab as set forth in FIG. 12C (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N80, Q121, and/or N171 of the heavy chain (SEQ ID NO:49) or N162 and/or N214 of the light chain (SEQ ID NO:50). Alternatively or in addition to, the HuPTM mAb or antigen binding-fragment thereof with the heavy and light chain variable domain sequences of golimumab has a sulfation group at Y112, Y113 and/or Y114 of the heavy chain (SEQ ID NO:49) and/or Y89 and/or Y90 of the light chain (SEQ ID NO:50). In other embodiments, the anti-TNFα HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of or relieve one or more symptoms of non-infectious uveitis, such as to reduce the levels of pain, redness of the eye, sensitivity to light, and/or other discomfort for the patient. Efficacy may be monitored by measuring a reduction in pain, redness of the eye, and/or photophobia and/or an improvement in vision.

Combinations of delivery of the anti-TNFα HuPTM mAb or antigen-binding fragment thereof, to the liver or muscles accompanied by delivery of other available treatments are encompassed by the methods provided herein. The additional treatments may be administered before, concurrently, or subsequent to the gene therapy treatment. Available treatments for a subject with non-infectious uveitis that could be combined with the gene therapy provided herein include but are not limited to, azathioprine, methotrexate, mycophenolate mofetil, cyclosporine, cyclophosphamide, corticosteroids (local and/or systemic), and others and administration with anti-TNFα agents, including but not limited to adalimumab, infliximab, or golimumab.

### 5.3.12. Anti-RGMa HuPTM Constructs and Formulations for Multiple Sclerosis

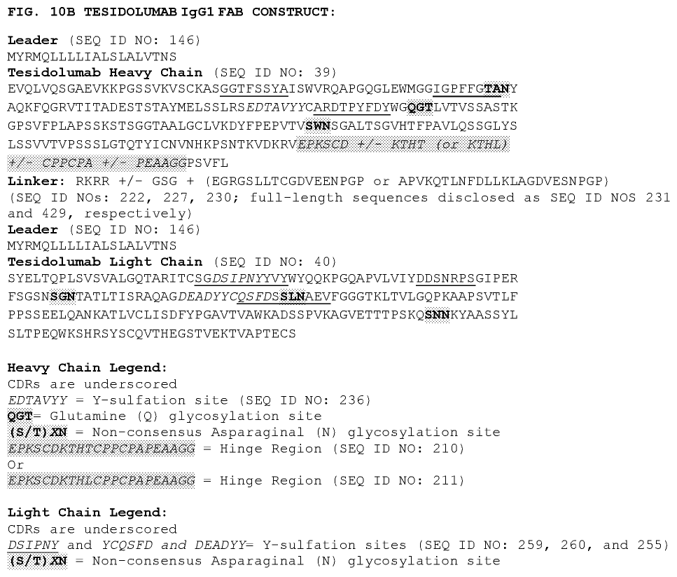

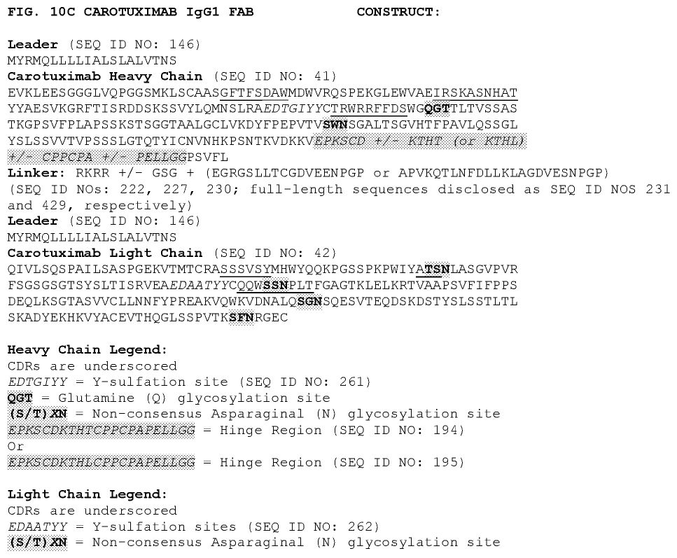

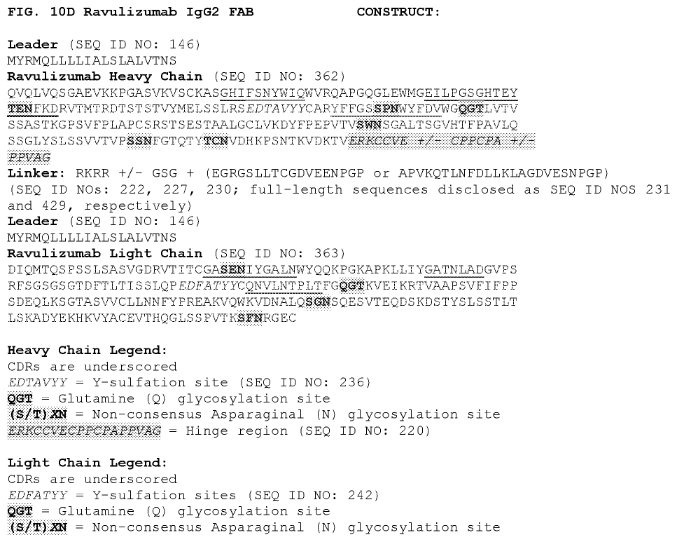

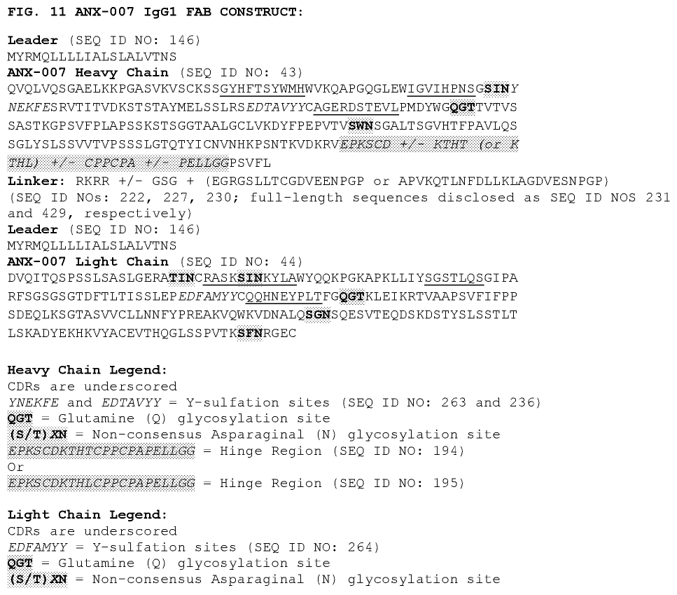

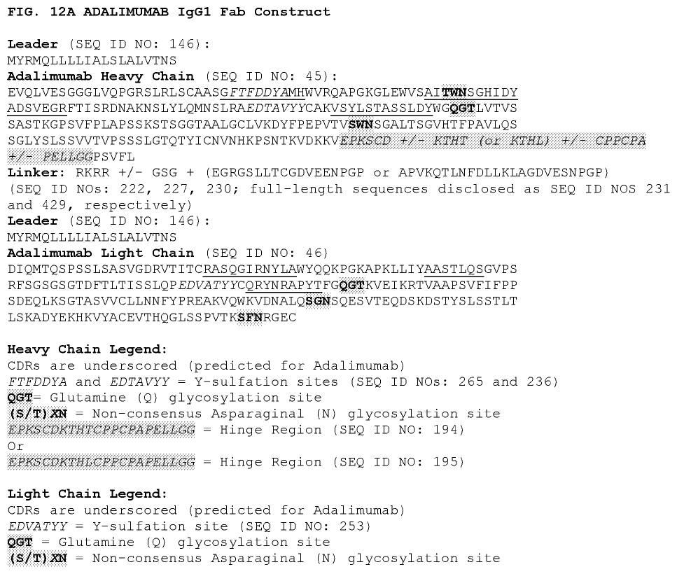

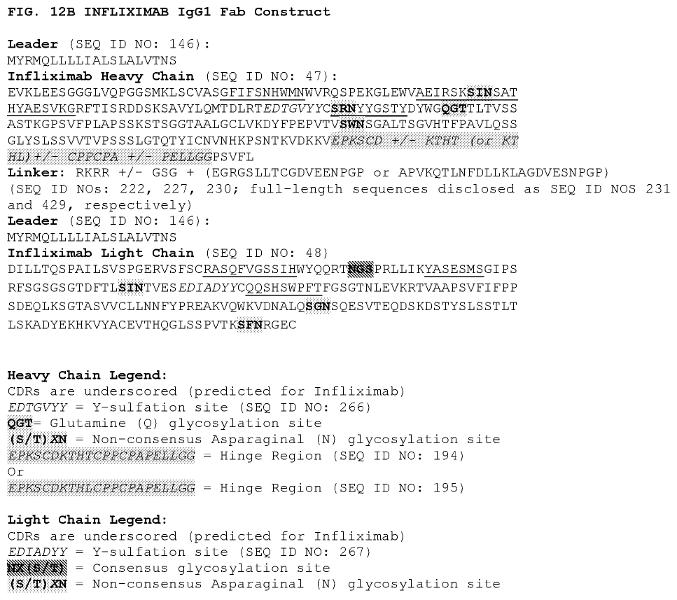

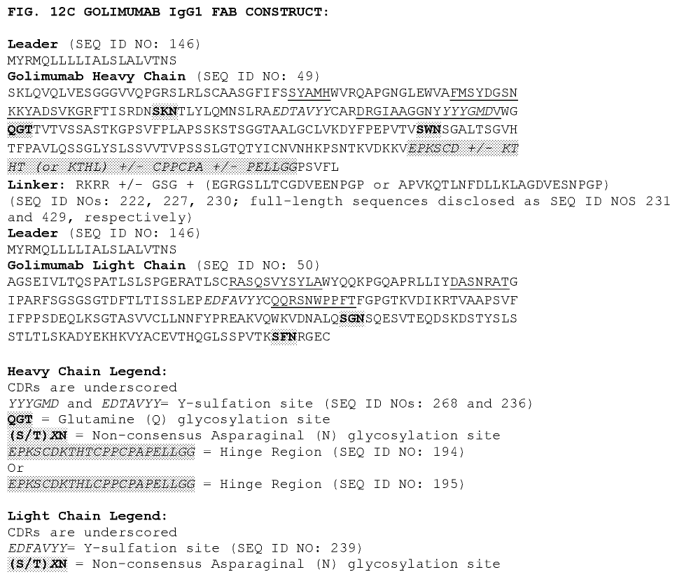

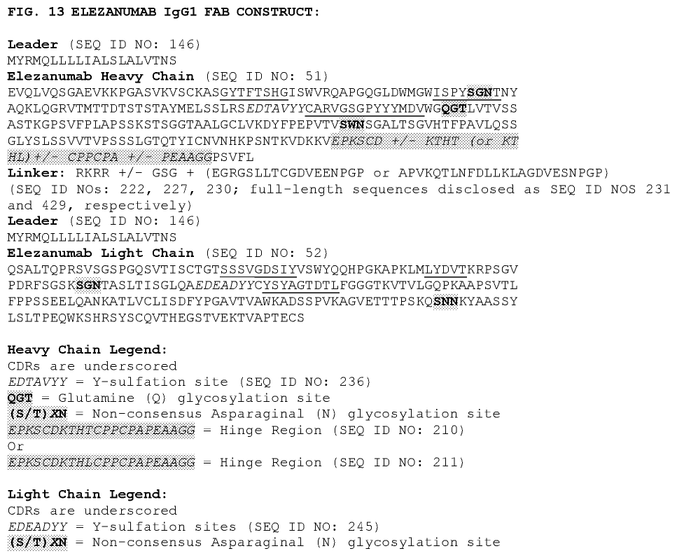

Compositions and methods are described for the delivery of HuPTM mAbs and antigen-binding fragments thereof, such as HuPTM Fabs, that bind to repulsive guidance molecule-A (RGMa) and indicated for treating multiple sclerosis (MS). In certain embodiments, the HuPTM mAb has the amino acid sequence of elezanumab or an antigen binding fragment of the foregoing. The amino acid sequence of Fab fragments of elezanumab is provided in FIG. 13. Delivery may be accomplished via gene therapy—e.g., by administering a viral vector or other DNA expression construct encoding an RGMa-binding HuPTM mAb (or an antigen binding fragment and/or a hyperglycosylated derivative or other derivative, thereof) to patients (human subjects) diagnosed with, or having one or more symptoms of MS to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to RGMa that can be administered to deliver the HuPTM mAb or antigen binding fragment in a patient. The transgene is a nucleic acid comprising the nucleotide sequences encoding an antigen binding fragment of an antibody that binds to RGMa, such as elezanumab, or variants thereof as detailed herein. The transgene may also encode an RGMa-integrin antigen binding fragment that contains additional glycosylation sites (e.g., see Courtois et al.).

In certain embodiments, the anti-RGMa antigen-binding fragment transgene comprises the nucleotide sequences encoding the heavy and light chains of the Fab portion of elezanumab (having amino acid sequences of SEQ ID NOs. 51 and 52, respectively, see Table 5 and FIG. 13). The nucleotide sequences may be codon optimized for expression in human cells. Nucleotide sequences may, for example, comprise the nucleotide sequences of SEQ ID NO: 121 (encoding the elezanumab heavy chain Fab portion) and SEQ ID NO: 122 (encoding the elezanumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} sequences, the transgenes may comprise, at the C-terminus of the heavy chain C_{H}1 domain sequence, all or a portion of the hinge region. In specific embodiments, the anti-integrin-antigen binding domain has a heavy chain Fab domain of SEQ ID NO: 51 with additional hinge region sequence starting after the C-terminal valine (V), contains all or a portion of the amino acid sequence EPKSCDKTHTCPPCPAPEAAGG (SEQ ID NO: 210), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPEAAGGPSVFL (SEQ ID NO: 212) or EPKSCDKTHLCPPCPAPEAAGGPSVFL (SEQ ID NO: 213) as set forth in FIG. 13. These hinge regions may be encoded by nucleotide sequences at the 3′ end of SEQ ID NO: 121 by the hinge region encoding sequences set forth in Table 6 (SEQ ID NO: 121). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 306 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-integrin antigen-binding fragment transgene encodes an RGMa antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 52. In certain embodiments, the anti-RGMa antigen-binding fragment transgene encodes an integrin antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 51. In certain embodiments, the anti-RGMa antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 52 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 51. In specific embodiments, the RGMa antigen binding fragment comprises a heavy chain comprising an amino acid sequence of SEQ ID NO: 51 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIG. 13) or are substitutions with an amino acid present at that position in the heavy chain of one or more of the other therapeutic antibodies, for example, as identified by the alignment in FIG. 20A. In specific embodiments, the integrin antigen binding fragment comprises a light chain comprising an amino acid sequence of SEQ ID NO: 52 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 13) or are substitutions with an amino acid present at that position in the light chain of one or more of the other therapeutic antibodies, for example, as identified by the alignment in FIG. 20B.

In certain embodiments, the anti-RGMa antigen-binding fragment transgene encodes a hyperglycosylated elezanumab Fab, comprising a heavy chain and a light chain of SEQ ID NOs: 51 and 52, respectively, with one or more of the following mutations: L115N (heavy chain) and/or Q197N (light chain) (see FIGS. 20A (heavy chain) and 20B (light chain)).

In certain embodiments, the anti-RGMa antigen-binding fragment transgene encodes an antigen-binding fragment and comprises the nucleotide sequences encoding the six elezanumab CDRs which are underlined in the heavy and light chain variable domain sequences of FIG. 13 which are spaced between framework regions, generally human framework regions, and associated with constant domains depending upon the form of the antigen-binding molecule, as is known in the art to form the heavy and/or light chain variable domain of an anti-RGMa antibody or antigen-binding fragment thereof.

In specific embodiments, provided are AAV vectors comprising a viral capsid that is at least 95% identical to the amino acid sequence of an AAV8 capsid (SEQ ID NO: 72), AAV9 capsid (SEQ ID NO: 73) or AAVrh10 capsid (SEQ ID NO: 74); and an artificial genome comprising an expression cassette flanked by AAV inverted terminal repeats (ITRs), wherein the expression cassette comprises a transgene encoding an anti-RGMa mAb, or an antigen-binding fragment thereof, operably linked to one or more regulatory sequences that control expression of the transgene in human liver or muscle cells.

Gene Therapy Methods

Provided are methods of treating human subjects for MS by administration of a viral vector containing a transgene encoding an anti-RGMa antibody, or antigen binding fragment thereof. The antibody may be elenazumab and is, for example, a full length antibody or Fab fragment thereof, or other antigen-binding fragment thereof. In embodiments, the patient has been diagnosed with and/or has symptoms associated with MS. Recombinant vector used for delivering the transgene are described in Section 5.4.1 and 5.4.2. In some embodiments, such vectors should have a tropism for human liver cells and can include non-replicating rAAV, particularly those bearing an AAV8 or AAV9 capsid. The recombinant vectors, such as those shown in FIG. 13, can be administered in any manner such that the recombinant vector enters the liver or muscle tissue, e.g. by introducing the recombinant vector into the bloodstream. See 5.5.2 for details regarding the methods of treatment. In other embodiments, such vectors should have a tropism for human CNS cells and can include non-replicating rAAV, particularly those bearing an AAV9, AAVrh10, AAVrh20, AAVrh39, or AAVcy5 capsid. The recombinant vector, such as shown in FIG. 13, can be administered in any manner such that the recombinant vector enters the CNS, e.g. by introducing the recombinant vector into the cerebral spinal fluid (CSF). See Section 5.5.1 for details regarding the methods of treatment.

Subjects to whom such gene therapy is administered can be those responsive to anti-RGMa therapy. In certain embodiments, the methods encompass treating patients who have been diagnosed with MS, or have one or more symptoms associated therewith, and identified as responsive to treatment with an anti-RGMa antibody or considered a good candidate for therapy with an anti-RGMa antibody. In specific embodiments, the patients have previously been treated with elezanumab, and have been found to be responsive to elezanumab. To determine responsiveness, the anti-RGMa antibody or antigen-binding fragment transgene product (e.g., produced in cell culture, bioreactors, etc.) may be administered directly to the subject.

Human Post Translationally Modified Antibodies

The production of the anti-RGMa HuPTM mAb or HuPTM Fab, should result in a “biobetter” molecule for the treatment of MS accomplished via gene therapy—e.g., by administering a viral vector or other DNA expression construct encoding the anti-RGMa HuPTM Fab, subcutaneously, intramuscularly, or intravenously to human subjects (patients) diagnosed with or having one or more symptoms of MS, to create a permanent depot in the liver, muscle or CNS tissue that continuously supplies the fully-human post-translationally modified, such as human-glycosylated, sulfated transgene product produced by transduced liver, muscle or CNS cells.

The cDNA construct for the anti-RGMa HuPTMmAb or anti-RGMa HuPTM Fab should include a signal peptide that ensures proper co- and post-translational processing (glycosylation and protein sulfation) by the transduced liver or muscle cells. For example, the signal sequence may be MYRMQLLLLIALSLALVTNS (SEQ ID NO: 146). Alternatively, in some embodiments, the signal sequence may have an amino acid sequence selected from any one of the signal sequences set forth in Tables 2, 3 or 4 that correspond to the proteins secreted by CNS cells, myocytes or hepatocytes, respectively.

As an alternative, or an additional treatment to gene therapy, the anti-RGMa HuPTM mAb or HuPTM Fab can be produced in human cell lines by recombinant DNA technology, and administered to patients diagnosed with MS, or for whom therapy for MS is considered appropriate.

In specific embodiments, the anti-RGMa HuPTM mAb or antigen-binding fragment thereof has heavy and light chains with the amino acid sequences of the heavy and light chain Fab portions of elezanumab as set forth in FIG. 13 (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N57, Q112, and/or N162 of the heavy chain (SEQ ID NO:51) or N71 and/or N173 of the light chain (SEQ ID NO:52). Alternatively or in addition to, the HuPTM mAb or antigen binding-fragment thereof with the heavy and light chain variable domain sequences of elezanumab has a sulfation group at Y94 and/or Y95 of the heavy chain (SEQ ID NO: 51) and/or Y88 and/or Y89 of the light chain (SEQ ID NO: 52). In other embodiments, the anti-RGMa HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of MS, particularly a reduction in pain and discomfort for the patient and/or improvements in mobility. Efficacy may be monitored by scoring the symptoms or degree of lesions in the affected tissue. For example, with regard to MS, efficacy can be monitored by assessing frequency of relapses (e.g., Annualized Relapse Rate), physical disability status (e.g., scoring Kurtzke Expanded Disability Status Scale (EDSS)), and biological markers, including brain scans using MRI (e.g., evaluation of T1-weighted gadolinium (Gd)-enhancing lesions and T2-hyperintense lesions through magnetic resonance imaging).

Combinations of delivery of the anti-RGMa HuPTM mAb or antigen-binding fragment thereof, to the CNS, liver, or muscles accompanied by delivery of other available treatments are encompassed by the methods provided herein. The additional treatments may be administered before, concurrently, or subsequent to the gene therapy treatment. Available treatments for MS that could be combined with the gene therapy provided herein include but are not limited to interferon beta, interferon beta 1a, glatiramer acetate, cyclophosphamide, corticosteroids, immunomodulators (e.g, azathioprine, 6-mercaptopurine, and/or methotrexate), and mitoxantrone and administration with anti-RGMa agents, including but not limited to elenazumab.

### 5.3.13 Anti-TTR HuPTM Constructs and Formulations for Amyloidosis

Compositions and methods are described for the delivery of HuPTM mAbs and antigen-binding fragments thereof, such as HuPTM Fabs, that bind to transthyretin (TTR), particularly mis-folded or pathogenic TTR, indicated for treating familial or wild-type amyloido transthyretin (ATTR) amyloidosis, familial amyloid cardiomyopathy (FAC), and/or familial amyloid polyneuropathy (FAP). In certain embodiments, the HuPTM mAb has the amino acid sequence of NI-301 or PRX-004 or an antigen binding fragment thereof. The amino acid sequence of Fab fragments of NI-301 and PRX-004 are provided in FIGS. 14A and 14B, respectively. Delivery may be accomplished via gene therapy—e.g., by administering a viral vector or other DNA expression construct encoding a TTR-binding HuPTM mAb (or an antigen binding fragment and/or a hyperglycosylated derivative or other derivative, thereof) to patients (human subjects) diagnosed with, or having one or more symptoms of amyloidosis, FAP, and/or FAC to create a permanent depot that continuously supplies the human PTM, e.g., human-glycosylated, transgene product.

Provided are recombinant vectors containing a transgene encoding a HuPTM mAb or HuPTM Fab (or other antigen binding fragment of the HuPTM mAb) that binds to TTR that can be administered to deliver the HuPTM mAb or antigen binding fragment in a patient. The transgene is a nucleic acid comprising the nucleotide sequences encoding an antigen binding fragment of an antibody that binds to TTR, such as NI-301, PRX-004, or variants thereof as detailed herein. The transgene may also encode an anti-TTR antigen binding fragment that contains additional glycosylation sites (e.g., see Courtois et al.).

In certain embodiments, the anti-TTR antigen-binding fragment transgene comprises the nucleotide sequences encoding the heavy and light chains of the Fab portion of NI-301 (having amino acid sequences of SEQ ID NOs. 53 and 54, respectively, see Table 5 and FIG. 14A). The nucleotide sequences may be codon optimized for expression in human cells. Nucleotide sequences may, for example, comprise the nucleotide sequences of SEQ ID NO: 123 (encoding the NI-301 heavy chain Fab portion) and SEQ ID NO: 124 (encoding the NI-301 light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-TTR antigen binding domain has a heavy chain Fab domain of SEQ ID NO: 53 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 14A. These hinge regions may be encoded by nucleotide sequences at the 3′ end of SEQ ID NO: 123 by the hinge region encoding sequences set forth in Table 6 (SEQ ID NO: 123). In another embodiment, the transgenes comprise the amino acid sequences encoding the full length (or substantially full length) heavy and light chains of the antibody, comprising the Fc domain at the C terminus of the heavy chain, e.g., an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-TTR antigen-binding fragment transgene encodes an TTR antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 54. In certain embodiments, the anti-TTR antigen-binding fragment transgene encodes an TTR antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 53. In certain embodiments, the anti-TTR antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 54 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 53. In specific embodiments, the TTR antigen binding fragment comprises a heavy chain comprising an amino acid sequence of SEQ ID NO: 53 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIG. 14A) or are substitutions with an amino acid present at that position in the heavy chain of one or more of the other therapeutic antibodies, for example, as identified by the alignment in FIG. 20A. In specific embodiments, the TTR antigen binding fragment comprises a light chain comprising an amino acid sequence of SEQ ID NO: 54 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 14A) or are substitutions with an amino acid present at that position in the light chain of one or more of the other therapeutic antibodies, for example, as identified by the alignment in FIG. 20B.

In certain embodiments, the anti-TTR antigen-binding fragment transgene encodes a hyperglycosylated NI-301 Fab, comprising a heavy chain and a light chain of SEQ ID NOs: 53 and 54, respectively, with one or more of the following mutations: M115N (heavy chain), Q159N or Q159S (light chain), and/or E194N (light chain) (see FIGS. 20A (heavy chain) and 20B (light chain)).

In certain embodiments, the anti-TTR antigen-binding fragment transgene encodes an antigen-binding fragment and comprises the nucleotide sequences encoding the six NI-301 CDRs which are underlined in the heavy and light chain variable domain sequences of FIG. 14A which are spaced between framework regions, generally human framework regions, and associated with constant domains depending upon the form of the antigen-binding molecule, as is known in the art to form the heavy and/or light chain variable domain of an anti-TTR antibody or antigen-binding fragment thereof.

In certain embodiments, the anti-TTR antigen-binding fragment transgene comprises the nucleotide sequences encoding the heavy and light chains of the Fab portion of PRX-004 (having amino acid sequences of SEQ ID NOs. 55 and 56, respectively, see Table 5 and FIG. 14B). The nucleotide sequences may be codon optimized for expression in human cells. Nucleotide sequences may, for example, comprise the nucleotide sequences of SEQ ID NO: 125 (encoding the PRX-004 heavy chain Fab portion) and SEQ ID NO: 126 (encoding the PRX-004 light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-TTR antigen binding domain has a heavy chain Fab domain of SEQ ID NO: 55 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 14B. These hinge regions may be encoded by nucleotide sequences at the 3′ end of SEQ ID NO: 125 by the hinge region encoding sequences set forth in Table 6 (SEQ ID NO: 125). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 307 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-TTR antigen-binding fragment transgene encodes an TTR antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 56. In certain embodiments, the anti-TTR antigen-binding fragment transgene encodes an TTR antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 55. In certain embodiments, the anti-TTR antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 56 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 55. In specific embodiments, the TTR antigen binding fragment comprises a heavy chain comprising an amino acid sequence of SEQ ID NO: 55 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 14B) or are substitutions with an amino acid present at that position in the heavy chain of one or more of the other therapeutic antibodies, for example, as identified by the alignment in FIG. 20A. In specific embodiments, the TTR antigen binding fragment comprises a light chain comprising an amino acid sequence of SEQ ID NO: 56 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 14B) or are substitutions with an amino acid present at that position in the light chain of one or more of the other therapeutic antibodies, for example, as identified by the alignment in FIG. 20B.

In certain embodiments, the anti-TTR antigen-binding fragment transgene encodes a hyperglycosylated PRX-004 Fab, comprising a heavy chain and a light chain of SEQ ID NOs: 55 and 56, respectively, with one or more of the following mutations: L112N (heavy chain), Q160N or Q160S (light chain), and/or E195N (light chain) (see FIGS. 20A (heavy chain) and 20B (light chain)).

In certain embodiments, the anti-TTR antigen-binding fragment transgene encodes an antigen-binding fragment and comprises the nucleotide sequences encoding the six PRX-004 CDRs which are underlined in the heavy and light chain variable domain sequences of FIG. 14B which are spaced between framework regions, generally human framework regions, and associated with constant domains depending upon the form of the antigen-binding molecule, as is known in the art to form the heavy and/or light chain variable domain of an anti-TTR antibody or antigen-binding fragment thereof.

Gene Therapy Methods

Provided are methods of treating human subjects for ATTR, FAT, or FAC by administration of a viral vector containing a transgene encoding an anti-TTR antibody or antigen binding fragment thereof. The antibody may be NI-301, PRX-004 and is, e.g., a full length antibody or Fab fragment thereof, or other antigen-binding fragment thereof. In embodiments, the patient has been diagnosed with and/or has symptoms associated with ATTR, FAC, or FAT.

Provided are methods of treating human subjects for ATTR, FAC, and FAT by administration of a viral vector containing a transgene encoding an anti-TTR antibody, or antigen binding fragment thereof. The antibody may be NI-301, or PRX-004, and is, e.g., a Fab fragment thereof, or other antigen-binding fragment thereof. In embodiments, the patient has been diagnosed with and/or has symptoms associated with ATTR, FAT, or FAC. Recombinant vector used for delivering the transgene are described in Section 5.4.1 and 5.4.2. In some embodiments, such vectors should have a tropism for human liver cells and can include non-replicating rAAV, particularly those bearing an AAV8 or AAV9 capsid. The recombinant vectors, such as those shown in FIGS. 14A and 14B, can be administered in any manner such that the recombinant vector enters the liver or muscle tissue, e.g., by introducing the recombinant vector into the bloodstream. See 5.5.2 for details regarding the methods of treatment. In other embodiments, such vectors should have a tropism for human CNS cells and can include non-replicating rAAV, particularly those bearing an AAV9, AAVrh10, AAVrh20, AAVrh39, or AAVcy5 capsid. The recombinant vector, such as shown in FIGS. 14A and 14B, can be administered in any manner such that the recombinant vector enters the CNS, e.g., by introducing the recombinant vector into the cerebral spinal fluid (CSF). See Section 5.5.1 for details regarding the methods of treatment.

Subjects to whom such gene therapy is administered can be those responsive to anti-TTR therapy. In certain embodiments, the methods encompass treating patients who have been diagnosed with ATTR, FAP, or FAC, or have one or more symptoms associated therewith, and identified as responsive to treatment with an anti-TTR antibody, or considered a good candidate for therapy with an anti-TTR antibody. In specific embodiments, the patients have previously been treated with PRX-004 or NI-301, and have been found to be responsive to PRX-004 or NI-301. To determine responsiveness, the anti-TTR or antigen-binding fragment transgene product (e.g., produced in cell culture, bioreactors, etc.) may be administered directly to the subject.

Human Post Translationally Modified Antibodies

The production of the anti-TTR HuPTM mAb or HuPTM Fab, should result in a “biobetter” molecule for the treatment of ATTR, FAC, or FAP accomplished via gene therapy—e.g., by administering a viral vector or other DNA expression construct encoding the anti-TTR HuPTM Fab, subcutaneously, intramuscularly, or intravenously to human subjects (patients) diagnosed with ATTR, FAC, or FAP, to create a permanent depot in the muscle or liver that continuously supplies the fully-human post-translationally modified, e.g., human-glycosylated, sulfated transgene product produced by transduced cells of the muscle or liver.

The cDNA construct for the anti-TTR HuPTMmAb or anti-TTR HuPTM Fab should include a signal peptide that ensures proper co- and post-translational processing (glycosylation and protein sulfation) by the transduced cells of the muscle or liver. For example, the signal sequence may be MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by cells of the muscle or liver, respectively.

As an alternative, or an additional treatment to gene therapy, the anti-TTR HuPTM mAb or HuPTM Fab can be produced in human cell lines by recombinant DNA technology, and administered to patients diagnosed with ATTR, FAP, or FAC is considered appropriate.

In specific embodiments, the anti-TTR HuPTM mAb or antigen-binding fragment thereof has heavy and light chains with the amino acid sequences of the heavy and light chain Fab portions of NI-301 as set forth in FIG. 14A (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N58, N78, N83, Q112, and/or N161 of the heavy chain (SEQ ID NO: 53) or Q99, N157, and/or N209 of the light chain (SEQ ID NO: 54). Alternatively or in addition to, the HuPTM mAb or antigen binding-fragment thereof with the heavy and light chain variable domain sequences of NI-301 has a sulfation group at Y95 and/or Y96 of the heavy chain (SEQ ID NO: 53) and/or Y85 and/or Y86 of the light chain (SEQ ID NO: 54). In other embodiments, the anti-TTR HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-TTR HuPTM mAb or antigen-binding fragment thereof has heavy and light chains with the amino acid sequences of the heavy and light chain Fab portions of PRX-004 as set forth in FIG. 14B (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N76, Q109, and/or N159 of the heavy chain (SEQ ID NO: 55) or N158 and/or N210 of the light chain (SEQ ID NO: 56). Alternatively or in addition to, the HuPTM mAb or antigen binding-fragment thereof with the heavy and light chain variable domain sequences of PRX-004 has a sulfation group at Y93 and/or Y94 of the heavy chain (SEQ ID NO: 55) and/or Y86 and/or Y87 of the light chain (SEQ ID NO: 56). In other embodiments, the anti-TTR HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of the disease being treated or alleviate one or more symptoms thereof. In the case of ATTR, efficacy can be monitored by assessing one or more amyloidosis endpoints, including by measuring the progression of organ damage, the amount of amyloid fibril tissue deposits, and/or improvement in organ function (i.e. kidney and liver).

Combinations of delivery of the anti-TTR HuPTM mAb or antigen-binding fragment thereof to the muscle or liver accompanied by delivery of other available treatments are encompassed by the methods provided herein. The additional treatments may be administered before, concurrently, or subsequent to the gene therapy treatment. Available treatments for ATTR that could be combined with the gene therapy provided herein include but are not limited to chemotherapeutic agent(s) (e.g., alkylating agents, antimetabolites, topoisomerase inhibitors, and mitotic inhibitors), lenalidomide (REVLIMID®), pomalidomide (POMALYST®), thalidomide (SYNOVIR®), daraumumab (DARZALEX®), elotuzumab (EMPLICITI®), bortezomib (VELCADE®), ixazomib (NINLARO®), and/or carfilzomib (KYPROLIS®) and administration with anti-TTR agents, including but not limited to NI-301 and PRX-004.

### 5.3.14 Anti-CTGF HuPTM Constructs and Formulations for Fibrotic Diseases

Compositions and methods are described for the delivery of HuPTM mAbs and antigen-binding fragments thereof, such as HuPTM Fabs, that bind to connective tissue growth factor (CTGF) indicated for treating one or more fibrotic disorders including pulmonary fibrosis, cystic fibrosis (CF), idiopathic pulmonary fibrosis (IPF), liver cirrhosis, atrial fibrosis, endomyocardial fibrosis, old myocardial infarction, arthrofibrosis, Crohn's disease, mediastinal fibrosis, myelofibrosis (MF), nephrogenic systemic fibrosis (NSF), progressive massive fibrosis (PMF), retroperitoneal fibrosis (RPF). In certain embodiments, the HuPTM mAb has the amino acid sequence of pamrevlumab or an antigen binding fragment thereof. The amino acid sequence of Fab fragments of pamrevlumab is provided in FIG. 15. Delivery may be accomplished via gene therapy—e.g., by administering a viral vector or other DNA expression construct encoding an CTGF-binding HuPTM mAb (or an antigen binding fragment and/or a hyperglycosylated derivative or other derivative, thereof) to patients (human subjects) diagnosed with, or having one or more symptoms of a fibrotic disorder to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to CTGF that can be administered to deliver the HuPTM mAb or antigen binding fragment in a patient. The transgene is a nucleic acid comprising the nucleotide sequences encoding an antigen binding fragment of an antibody that binds to CTGF, such as pamrevlumab, or variants thereof as detailed herein. The transgene may also encode an anti-CTGF antigen binding fragment that contains additional glycosylation sites (e.g., see Courtois et al.).

In certain embodiments, the anti-CTGF antigen-binding fragment transgene comprises the nucleotide sequences encoding the heavy and light chains of the Fab portion of pamrevlumab (having amino acid sequences of SEQ ID NOs. 57 and 58, respectively, see Table 5 and FIG. 15). The nucleotide sequences may be codon optimized for expression in human cells Nucleotide sequences may, for example, comprise the nucleotide sequences of SEQ ID NO: 127 (encoding the pamrevlumab heavy chain Fab portion) and SEQ ID NO: 128 (encoding the pamrevlumab light chain Fab portion) as set forth in Table 6. In the case of treating fibrotic diseases, the heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-CTGF antigen binding domain has a heavy chain Fab domain of SEQ ID NO: 57 with additional hinge region sequence starting after the C-terminal valine (V), contains all or a portion of the amino acid sequence EPKSCDKTHTCPPCPAPEAAGG (SEQ ID NO: 210), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPEAAGGPSVFL (SEQ ID NO: 212) or EPKSCDKTHLCPPCPAPEAAGGPSVFL (SEQ ID NO: 213) as set forth in FIG. 15. These hinge regions may be encoded by nucleotide sequences at the 3′ end of SEQ ID NO: 127 by the hinge region encoding sequences set forth in Table 6 (SEQ ID NO: 127). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 308 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-CTGF antigen-binding fragment transgene encodes an CTGF antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 58. In certain embodiments, the anti-CTGF antigen-binding fragment transgene encodes an CTGF antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 57. In certain embodiments, the anti-CTGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 58 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 57. In specific embodiments, the CTGF antigen binding fragment comprises a heavy chain comprising an amino acid sequence of SEQ ID NO: 57 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 15) or are substitutions with an amino acid present at that position in the heavy chain of one or more of the other therapeutic antibodies, for example, as identified by the alignment in FIG. 20A. In specific embodiments, the CTGF antigen binding fragment comprises a light chain comprising an amino acid sequence of SEQ ID NO: 58 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 15) or are substitutions with an amino acid present at that position in the light chain of one or more of the other therapeutic antibodies, for example, as identified by the alignment in FIG. 20B.

In certain embodiments, the anti-CTGF antigen-binding fragment transgene encodes a hyperglycosylated pamrevlumab Fab, comprising a heavy chain and a light chain of SEQ ID NOs: 57 and 58, respectively, with one or more of the following mutations: L111N (heavy chain) and/or Q196N (light chain) (see FIGS. 20A (heavy chain) and 20B (light chain)).

In certain embodiments, the anti-CTGF antigen-binding fragment transgene encodes an antigen-binding fragment and comprises the nucleotide sequences encoding the six pamrevlumab CDRs which are underlined in the heavy and light chain variable domain sequences of FIG. 15 which are spaced between framework regions, generally human framework regions, and associated with constant domains depending upon the form of the antigen-binding molecule, as is known in the art to form the heavy and/or light chain variable domain of an anti-CTGF antibody or antigen-binding fragment thereof.

Gene Therapy Methods

Provided are methods of treating human subjects for one or more fibrotic disorders (such as IPF) by administration of a viral vector containing a transgene encoding an anti-CTGF antibody or antigen binding fragment thereof. The antibody may be pamrevlumab, and is e.g. a full length or substantially full length antibody or Fab fragment thereof, or other antigen-binding fragment thereof. In embodiments, the patient has been diagnosed with and/or has symptoms associated with one or more fibrotic disorders.

Recombinant vector used for delivering the transgene are described in Section 5.4.3. For delivery to the liver, recombinant vector used for delivering the transgene are described in Section 5.4.2. Such vectors should have a tropism for human liver cells and can include non-replicating rAAV, particularly those bearing an AAV8 or AAV9 capsid. The recombinant vectors, such as those shown in FIG. 15, can be administered in any manner such that the recombinant vector enters the liver, e.g. by introducing the recombinant vector into the bloodstream. See Section 5.5.2 for details regarding the methods of treatment.

Subjects to whom such gene therapy is administered can be those responsive to anti-CTGF therapy. In certain embodiments, the methods encompass treating patients who have been diagnosed with one or more fibrotic disorders, or have one or more symptoms associated therewith, and identified as responsive to treatment with an anti-CTGF antibody or considered a good candidate for therapy with an anti-CTGF antibody. In specific embodiments, the patients have previously been treated with pamrevlumab, and have been found to be responsive to pamrevlumab. To determine responsiveness, the anti-CTGF or antigen-binding fragment transgene product (e.g., produced in cell culture, bioreactors, etc.) may be administered directly to the subject.

### Human Post Translationally Modified Antibodies

The production of the anti-CTGF HuPTM mAb or HuPTM Fab, should result in a “biobetter” molecule for the treatment of one or more fibrotic disorders accomplished via gene therapy—e.g., by administering a viral vector or other DNA expression construct encoding the anti-CTGF HuPTM Fab, subcutaneously, intramuscularly, or intravenously to human subjects (patients) diagnosed with one or more fibrotic disorders, to create a permanent depot in the liver or muscle that continuously supplies the fully-human post-translationally modified, e.g., human-glycosylated, sulfated transgene product produced by transduced cells of the liver or muscle.

The cDNA construct for the anti-CTGF HuPTMmAb or anti-CTGF HuPTM Fab should include a signal peptide that ensures proper co- and post-translational processing (glycosylation and protein sulfation) by the transduced cells of the muscle or liver. For example, the signal sequence may be MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by cells of the muscle or liver, respectively.

As an alternative, or an additional treatment to gene therapy, the anti-CTGF HuPTM mAb or HuPTM Fab can be produced in human cell lines by recombinant DNA technology and administered to patients diagnosed with a fibrotic disorder for whom therapy for a fibrotic disorder is considered appropriate.

In specific embodiments, the anti-CTGF HuPTM mAb or antigen-binding fragment thereof has heavy and light chains with the amino acid sequences of the heavy and light chain Fab portions of pamrevlumab as set forth in FIG. 15 (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N59, Q108, and/or N158 of the heavy chain (SEQ ID NO:57) or N68, N95, and/or N172 of the light chain (SEQ ID NO:58). Alternatively or in addition to, the HuPTM mAb or antigen binding-fragment thereof with the heavy and light chain variable domain sequences of pamrevlumab has a sulfation group at Y94 and/or Y95 of the heavy chain (SEQ ID NO: 57) and/or Y30, Y85 and/or Y86 of the light chain (SEQ ID NO: 58). In other embodiments, the anti-CTGF HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of the disease being treated or alleviate one or more symptoms thereof. In the case of CF, efficacy can be monitored by assessing Forced Expiratory Volume in is (FEV1), decreased frequency of pulmonary exacerbations, quality of life (QoL) improvement, and, for younger patients, growth improvement. (e.g., see VanDevanter and Konstan, “Outcome measurement for clinical trials assessing treatment of cystic fibrosis lung disease,” Clin. Investig. 2(2):163-175 (2012)). In the case of pulmonary fibrosis, efficacy can be monitored by assessing the lung vital capacity (LVC), desaturation with exertion, and diffusing capacity of the lungs for carbon monoxide (DLCO). In the case of IPF, efficacy may be monitored by assessing the level of dyspnoea, FVC, DLCO, desaturation during the 6-min walk test, extent of honeycombing on high-resolution computed tomography (HRCT), or presence of pulmonary hypertension or emphysema. In the case of liver cirrhosis, efficacy may be monitored by assessing the hepatic venous pressure gradient (HVPG) and the stage of liver fibrosis. In the case of atrial fibrosis, efficacy may be monitored by localizing and quantifying the degree of structural remodeling (SRM) and/or fibrosis in the atrium using contrast-media enhanced delayed enhancement MRI (DE-MRI). In the case of endomyocardial fibrosis, efficacy may be monitored by assessing fibrotic lesions of the inner lining of the heart cavities. In the case of arthrofibrosis, efficacy may be monitored by assessing the extent of involvement by fibrosis in the affected joint (e.g., MRI). In the case of Crohn's disease, efficacy may be monitored by assessing the level of serological markers (e.g., fibronectin, CRP, bFGF, ASCA) and/or the degree of fibrotic lesions using advanced imaging technologies. In the case of mediastinal fibrosis, efficacy may be monitored by assessing the presence and/or degree of fibrosis, polymorphic inflammatory infiltrates and phlebitis by using contrast-enhanced CT. In the case of myelofibrosis, efficacy may be monitored by assessing changes in the level of inflammatory cytokine in the serum, blood counts (PLT, WBC, RBC), degree of fibrosis (reticulin and collagen) in the bone marrow, spleen size, and/or constitutional symptoms compared to baseline. In the case of nephrogenic systemic fibrosis, efficacy may be monitored by assessing changes in the glomerular filtration rate (GFR) from baseline, quantification of fibrotic and/or darkened of the skin, and/or reduction in burning, itching and/or severe sharp pains in areas of involvement. In the case of progressive massive fibrosis, efficacy may be monitored by improvement of pulmonary function and/or reduction in the size of conglomerate masses of dense fibrosis in the lungs. In the case of retroperitoneal fibrosis, the efficacy may be monitored by assessing decreased pain in the lower back and/or abdomen, improvement of anemia, abnormal discoloration of the skin, and/or quality of life.

Combinations of delivery of the anti-CTGF HuPTM mAb or antigen-binding fragment thereof to the muscle or liver accompanied by delivery of other available treatments are encompassed by the methods provided herein. The additional treatments may be administered before, concurrently, or subsequent to the gene therapy treatment. Available treatments for CF that could be combined with the gene therapy provided herein include but are not limited to antibiotics, vaccines, and cough medicines (e.g., acetylcysteine and dornasa alfa) and administration with anti-CTGF, including but not limited to pamrevlumab. Available treatments for IPF that could be combined with the gene therapy provided herein include but are not limited to oxygen therapy, pulmonary rehabilitation, Nintedanib (OFEV®), Pirfenidone (ESBRIET®, PIRFENEX®, PIRESPA®), Corticosteroids (prednisone), Mycophenolate mofetil/mycophenolic acid (CELLCEPT®), and Azathioprine (IMURAN®) and administration with anti-CTGF, including but not limited to pamrevlumab. Available treatments for cirrhosis that could be combined with gene therapy provided herein include but are not limited to diuretics (e.g., spironolactone, metolazone, and frusemide), ammonia reducer, beta blocker, synthetic hormones (e.g., octeotride), antibiotics, and antiviral drugs. Available treatments for atrial fibrosis that could be combined with gene therapy provided herein include but are not limited to physical ablation, beta blockers, blood thinners, calcium channel blockers, corticosteroids (e.g., prednisone), non-steroidal anti-inflammatory drugs, and antiarrhythmics (digoxin). Available treatments for endomyocardial fibrosis that could be combined with gene therapy provided herein include but are not limited to surgery, blood thinners, ACE inhibitors, corticosteroids (e.g., prednisone), non-steroidal anti-inflammatory drugs, diuretics (e.g., spironolactone, metolazone, and frusemide) or other anti-inflammatories, and antiarrhythmics (digoxin). Available treatments for old myocardial infarction that could be combined with gene therapy provided herein include but are not limited to beta blockers, blood thinners, statins, nitroglycerin, and ACE inhibitors. Available treatments for arthrofibrosis that could be combined with gene therapy provided herein include but are not limited to surgery, physical therapy, corticosteroid injections, non-steroidal anti-inflammatory drugs, and cryotherapy. Available treatments for Crohn's disease that could be combined with gene therapy provided herein include but are not limited to vitamin D, anti-inflammatory agents, non-steroidal anti-inflammatory agents, corticosteroids, immunosuppressants (e.g., Remicade®, adalimumab, methotrexate, mercaptopurine, or azathioprine), and antibiotics. Available treatments for mediastinal fibrosis that could be combined with gene therapy provided herein include but are not limited to tamoxifen, anti-inflammatory agents, non-steroidal anti-inflammatory agents (e.g., indocin), corticosteroids, immunosuppressants (e.g., Remicade®, adalimumab, methotrexate, mercaptopurine, or azathioprine). Available treatments for MF that could be combined with gene therapy provided herein include but are not limited to ruxolitinib, thalidomide, androgen therapy, blood transfusions, chemotherapeutics, and radiation treatment of the spleen. Available treatments for NSF that could be combined with gene therapy provided herein include but are not limited to oxygen therapy and bronchodilators. Available treatments for RPF that could be combined with gene therapy provided herein include but are not limited to corticosteroids, immunosuppressants (e.g, mycophenolate mofetil, methotrexate, azathioprine, or cyclophosphamide), and tamoxifen).

### 5.3.15. Anti-IL6R, Anti-IL6, and Anti-CD19 HuPTM Constructs and Formulations for NMO and Non-Infectious Uveitis and Detrimental Immune Response

Compositions and methods are described for the delivery of HuPTM mAbs and antigen-binding fragments thereof, such as HuPTM Fabs, that bind to interleukin-6 receptor (IL6R), interleukin-6 (IL6), or cluster of differentiation 19 (CD19), derived from an anti-IL6R, anti-IL6, or anti-CD19 antibody, such as satralizumab, sarilumab, tocilizumab, siltuximab, clazakizumab, sirukumab, olokizumab, gerilimzumab, or inebilizumab (FIGS. 16A-I), indicated for treating non-infectious uveitis, neuromyelitis optica (NMO), diabetic retinopathy (DR), or diabetic macular edema (DME). Also described are compositions and methods for the delivery of HuPTM mAbs and antigen-binding fragments thereof, such as HuPTM Fabs, that bind to interleukin-6 receptor (IL6R) or interleukin-6 (IL6) derived from an anti-IL6R, anti-IL6, or anti-CD19 antibody, such as satralizumab, sarilumab, tocilizumab, siltuximab, clazakizumab, sirukumab, olokizumab, or gerilimzumab (FIGS. 16A-H), indicated for treating, inhibiting or ameliorating a detrimental immune response, such as inflammation, cytokine release syndrome and the like associated with viral or bacterial infection or administration of an immune-promoting therapeutic, such as an immune-oncology antibody, protein or cell based therapy, such as a CAR-T therapy. In certain embodiments, the HuPTM mAb has the amino acid sequence of satralizumab, sarilumab, tocilizumab, siltuximab, clazakizumab, sirukumab, olokizumab, gerilimzumab, inebilizumab, or an antigen binding fragment thereof. Amino acid sequences of Fab fragments of the antibody are provided in FIGS. 16A-I. Delivery may be accomplished via gene therapy—e.g., by administering a viral vector or other DNA expression construct encoding an IL6R-binding, IL6-binding, or CD19-binding HuPTM mAb (or an antigen binding fragment and/or a hyperglycosylated derivative or other derivative, thereof) to patients (human subjects) diagnosed with one or more symptoms of non-infectious uveitis, NMO, DR, or DME, or alternatively in need of treating, inhibiting or ameliorating a detrimental immune response to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to IL6R, IL6, or CD19 that can be administered to deliver the HuPTM mAb or antigen binding fragment in a patient. The transgene is a nucleic acid comprising the nucleotide sequences encoding an antigen binding fragment of an antibody that binds to IL6R, IL6, or CD19, such as satralizumab, sarilumab, tocilizumab, siltuximab, clazakizumab, sirukumab, olokizumab, gerilimzumab, inebilizumab, or variants thereof as detailed herein. The transgene may also encode an anti-IL6R, IL6, or anti-CD19 antigen binding fragment that contains additional glycosylation sites (e.g., see Courtois et al.).

In certain embodiments, the anti-IL6R antigen-binding fragment transgene comprises the nucleotide sequences encoding the heavy and light chains of the Fab portion of satralizumab (having amino acid sequences of SEQ ID NOs. 59 and 60, respectively, see Table 5 and FIG. 16A). The nucleotide sequences may be codon optimized for expression in human cells. Nucleotide sequences may, for example, comprise the nucleotide sequences of SEQ ID NO: 129 (encoding the satralizumab heavy chain Fab portion) and SEQ ID NO: 130 (encoding the satralizumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL6R-antigen binding domain has a heavy chain Fab domain of SEQ ID NO: 59 with additional hinge region sequence starting after the C-terminal valine (V), contains all or a portion of the amino acid sequence ERKSCVECPPCPAPPVAG (SEQ ID NO: 433) or ERKSCVECPPCPA (SEQ ID NO: 434) as set forth in FIG. 16A. These hinge regions may be encoded by nucleotide sequences at the 3′ end of SEQ ID NO: 129 by the hinge region encoding sequences set forth in Table 6 (SEQ ID NO: 129). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 309 (Table 7) or an IgG2 Fc domain, such as SEQ ID No. 284 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL6R antigen-binding fragment transgene encodes an IL6R antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 60. In certain embodiments, the anti-IL6R antigen-binding fragment transgene encodes an IL6R antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 59. In certain embodiments, the anti-IL6R antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 60 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 59. In specific embodiments, the IL6R antigen binding fragment comprises a heavy chain comprising an amino acid sequence of SEQ ID NO: 59 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16A) or are substitutions with an amino acid present at that position in the heavy chain of one or more of the other therapeutic antibodies, for example, as identified by the alignment in FIG. 20A. In specific embodiments, the IL6R antigen binding fragment comprises a light chain comprising an amino acid sequence of SEQ ID NO: 60 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16A) or are substitutions with an amino acid present at that position in the light chain of one or more of the other therapeutic antibodies, for example, as identified by the alignment in FIG. 20B.

In certain embodiments, the anti-IL6R antigen-binding fragment transgene encodes a hyperglycosylated satralizumab Fab, comprising a heavy chain and a light chain of SEQ ID NOs: 59 and 60, respectively, with one or more of the following mutations: L114N (heavy chain), Q160N or Q160S (light chain), and/or E195N (light chain) (see FIGS. 20A (heavy chain) and 20B (light chain)).

In certain embodiments, the anti-IL6R antigen-binding fragment transgene encodes an antigen-binding fragment and comprises the nucleotide sequences encoding the six satralizumab CDRs which are underlined in the heavy and light chain variable domain sequences of FIG. 16A which are spaced between framework regions, generally human framework regions, and associated with constant domains depending upon the form of the antigen-binding molecule, as is known in the art to form the heavy and/or light chain variable domain of an anti-IL6R antibody or antigen-binding fragment thereof.

In certain embodiments, the anti-IL6R antigen-binding fragment transgene comprises the nucleotide sequences encoding the heavy and light chains of the Fab portion of sarilumab (having amino acid sequences of SEQ ID NOs. 61 and 62, respectively, see Table 5 and FIG. 16B). The nucleotide sequences may be codon optimized for expression in human cells. Nucleotide sequences may, for example, comprise the nucleotide sequences of SEQ ID NO: 131 (encoding the sarilumab heavy chain Fab portion) and SEQ ID NO: 132 (encoding the sarilumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL6R-antigen binding domain has a heavy chain Fab domain of SEQ ID NO: 61 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 16B. These hinge regions may be encoded by nucleotide sequences at the 3′ end of SEQ ID NO: 131 by the hinge region encoding sequences set forth in Table 6 (SEQ ID NO: 131). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 310 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL6R antigen-binding fragment transgene encodes an IL6R antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 62. In certain embodiments, the anti-IL6R antigen-binding fragment transgene encodes an IL6R antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 61. In certain embodiments, the anti-IL6R antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 62 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 61. In specific embodiments, the IL6R antigen binding fragment comprises a heavy chain comprising an amino acid sequence of SEQ ID NO: 61 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16B) or are substitutions with an amino acid present at that position in the heavy chain of one or more of the other therapeutic antibodies, for example, as identified by the alignment in FIG. 20A. In specific embodiments, the IL6R antigen binding fragment comprises a light chain comprising an amino acid sequence of SEQ ID NO: 62 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16B) or are substitutions with an amino acid present at that position in the light chain of one or more of the other therapeutic antibodies, for example, as identified by the alignment in FIG. 20B.

In certain embodiments, the anti-IL6R antigen-binding fragment transgene encodes a hyperglycosylated sarilumab Fab, comprising a heavy chain and a light chain of SEQ ID NOs: 61 and 62, respectively, with one or more of the following mutations: M111N (heavy chain), Q160N or Q160S (light chain), and/or E195N (light chain) (see FIGS. 20A (heavy chain) and 20B (light chain)).

In certain embodiments, the anti-IL6R antigen-binding fragment transgene encodes an antigen-binding fragment and comprises the nucleotide sequences encoding the six sarilumab CDRs which are underlined in the heavy and light chain variable domain sequences of FIG. 16B which are spaced between framework regions, generally human framework regions, and associated with constant domains depending upon the form of the antigen-binding molecule, as is known in the art to form the heavy and/or light chain variable domain of an anti-IL6R antibody or antigen-binding fragment thereof.

In certain embodiments, the anti-IL6R antigen-binding fragment transgene comprises the nucleotide sequences encoding the heavy and light chains of the Fab portion of tocilizumab (having amino acid sequences of SEQ ID NOs. 341 and 342, respectively, see Table 5 and FIG. 16H). The nucleotide sequences may be codon optimized for expression in human cells. Nucleotide sequences may, for example, comprise the nucleotide sequences of SEQ ID NO: 353 (encoding the tocilizumab heavy chain Fab portion) and SEQ ID NO: 354 (encoding the tocilizumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL6R-antigen binding domain has a heavy chain Fab domain of SEQ ID NO: 341 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDEPKSCDKTHTCPPCPAPELLGG KTHTCPPCPAPELLGGPSVFL (SEQ ID NO: 200) or EPKSCDKTHLCPPCPAPELLGGPSVFL (SEQ ID NO: 201) as set forth in FIG. 16H. These hinge regions may be encoded by nucleotide sequences at the 3′ end of SEQ ID NO: 353 by the hinge region encoding sequences set forth in Table 6 (SEQ ID NO: 353). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 359 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL6R antigen-binding fragment transgene encodes an IL6R antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 342. In certain embodiments, the anti-IL6R antigen-binding fragment transgene encodes an IL6R antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 341. In certain embodiments, the anti-IL6R antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 342 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 341. In specific embodiments, the IL6R antigen binding fragment comprises a heavy chain comprising an amino acid sequence of SEQ ID NO: 341 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16H) or are substitutions with an amino acid present at that position in the heavy chain of one or more of the other therapeutic antibodies, for example, as identified by the alignment in FIG. 20A. In specific embodiments, the IL6R antigen binding fragment comprises a light chain comprising an amino acid sequence of SEQ ID NO: 342 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16H) or are substitutions with an amino acid present at that position in the light chain of one or more of the other therapeutic antibodies, for example, as identified by the alignment in FIG. 20B.

In certain embodiments, the anti-IL6R antigen-binding fragment transgene encodes a hyperglycosylated tocilizumab Fab, comprising a heavy chain and a light chain of SEQ ID NOs: 341 and 342, respectively, with one or more of the following mutations: L115N (heavy chain), Q160N or Q160S (light chain), and/or E195N (light chain) (see FIGS. 20A (heavy chain) and 20B (light chain)).

In certain embodiments, the anti-IL6R antigen-binding fragment transgene encodes an antigen-binding fragment and comprises the nucleotide sequences encoding the six tocilizumab CDRs which are underlined in the heavy and light chain variable domain sequences of FIG. 16H which are spaced between framework regions, generally human framework regions, and associated with constant domains depending upon the form of the antigen-binding molecule, as is known in the art to form the heavy and/or light chain variable domain of an anti-IL6R antibody or antigen-binding fragment thereof.

In certain embodiments, the anti-IL6 antigen-binding fragment transgene comprises the nucleotide sequences encoding the heavy and light chains of the Fab portion of siltuximab (having amino acid sequences of SEQ ID NOs. 331 and 332, respectively, see Table 5 and FIG. 16C). The nucleotide sequences may be codon optimized for expression in human cells. Nucleotide sequences may, for example, comprise the nucleotide sequences of SEQ ID NO: 343 (encoding the siltuximab heavy chain Fab portion) and SEQ ID NO: 344 (encoding the siltuximab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL6-antigen binding domain has a heavy chain Fab domain of SEQ ID NO: 331 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 16C. These hinge regions may be encoded by nucleotide sequences at the 3′ end of SEQ ID NO: 343 by the hinge region encoding sequences set forth in Table 6 (SEQ ID NO: 343). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 355 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL6 antigen-binding fragment transgene encodes an IL6 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 332. In certain embodiments, the anti-IL6 antigen-binding fragment transgene encodes an IL6 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 331. In certain embodiments, the anti-IL6 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 332 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 331. In specific embodiments, the IL6 antigen binding fragment comprises a heavy chain comprising an amino acid sequence of SEQ ID NO: 331 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16C) or are substitutions with an amino acid present at that position in the heavy chain of one or more of the other therapeutic antibodies, for example, as identified by the alignment in FIG. 20A. In specific embodiments, the IL6 antigen binding fragment comprises a light chain comprising an amino acid sequence of SEQ ID NO: 332 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16C) or are substitutions with an amino acid present at that position in the light chain of one or more of the other therapeutic antibodies, for example, as identified by the alignment in FIG. 20B.

In certain embodiments, the anti-IL6 antigen-binding fragment transgene encodes a hyperglycosylated siltuximab Fab, comprising a heavy chain and a light chain of SEQ ID NOs: 331 and 332, respectively, with one or more of the following mutations: S114N (heavy chain), Q159N or Q159S (light chain), and/or E194N (light chain) (see FIGS. 20A (heavy chain) and 20B (light chain)).

In certain embodiments, the anti-IL6 antigen-binding fragment transgene encodes an antigen-binding fragment and comprises the nucleotide sequences encoding the six siltuximab CDRs which are underlined in the heavy and light chain variable domain sequences of FIG. 16C which are spaced between framework regions, generally human framework regions, and associated with constant domains depending upon the form of the antigen-binding molecule, as is known in the art to form the heavy and/or light chain variable domain of an anti-IL6 antibody or antigen-binding fragment thereof.

In certain embodiments, the anti-IL6 antigen-binding fragment transgene comprises the nucleotide sequences encoding the heavy and light chains of the Fab portion of clazakizumab (having amino acid sequences of SEQ ID NOs. 333 and 334, respectively, see Table 5 and FIG. 16D). The nucleotide sequences may be codon optimized for expression in human cells. Nucleotide sequences may, for example, comprise the nucleotide sequences of SEQ ID NO: 345 (encoding the clazakizumab heavy chain Fab portion) and SEQ ID NO: 346 (encoding the clazakizumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL6-antigen binding domain has a heavy chain Fab domain of SEQ ID NO: 333 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 16D. These hinge regions may be encoded by nucleotide sequences at the 3′ end of SEQ ID NO: 345 by the hinge region encoding sequences set forth in Table 6 (SEQ ID NO: 345). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 356 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL6 antigen-binding fragment transgene encodes an IL6 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 334. In certain embodiments, the anti-IL6 antigen-binding fragment transgene encodes an IL6 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 333. In certain embodiments, the anti-IL6 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 334 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 333. In specific embodiments, the IL6 antigen binding fragment comprises a heavy chain comprising an amino acid sequence of SEQ ID NO: 333 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16D) or are substitutions with an amino acid present at that position in the heavy chain of one or more of the other therapeutic antibodies, for example, as identified by the alignment in FIG. 20A. In specific embodiments, the IL6 antigen binding fragment comprises a light chain comprising an amino acid sequence of SEQ ID NO: 334 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16D) or are substitutions with an amino acid present at that position in the light chain of one or more of the other therapeutic antibodies, for example, as identified by the alignment in FIG. 20B.

In certain embodiments, the anti-IL6 antigen-binding fragment transgene encodes a hyperglycosylated clazakizumab Fab, comprising a heavy chain and a light chain of SEQ ID NOs: 333 and 334, respectively, with one or more of the following mutations: L115N (heavy chain), Q163N or Q163 S (light chain), and/or E198N (light chain) (see FIGS. 20A (heavy chain) and 20B (light chain)).

In certain embodiments, the anti-IL6 antigen-binding fragment transgene encodes an antigen-binding fragment and comprises the nucleotide sequences encoding the six clazakizumab CDRs which are underlined in the heavy and light chain variable domain sequences of FIG. 16D which are spaced between framework regions, generally human framework regions, and associated with constant domains depending upon the form of the antigen-binding molecule, as is known in the art to form the heavy and/or light chain variable domain of an anti-IL6 antibody or antigen-binding fragment thereof.

In certain embodiments, the anti-IL6 antigen-binding fragment transgene comprises the nucleotide sequences encoding the heavy and light chains of the Fab portion of sirukumab (having amino acid sequences of SEQ ID NOs. 335 and 336, respectively, see Table 5 and FIG. 16E). The nucleotide sequences may be codon optimized for expression in human cells. Nucleotide sequences may, for example, comprise the nucleotide sequences of SEQ ID NO: 347 (encoding the sirukumab heavy chain Fab portion) and SEQ ID NO: 348 (encoding the sirukumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL6-antigen binding domain has a heavy chain Fab domain of SEQ ID NO: 335 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 16E. These hinge regions may be encoded by nucleotide sequences at the 3′ end of SEQ ID NO: 347 by the hinge region encoding sequences set forth in Table 6 (SEQ ID NO: 347). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 357 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL6 antigen-binding fragment transgene encodes an IL6 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 336. In certain embodiments, the anti-IL6 antigen-binding fragment transgene encodes an IL6 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 335. In certain embodiments, the anti-IL6 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 336 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 335. In specific embodiments, the IL6 antigen binding fragment comprises a heavy chain comprising an amino acid sequence of SEQ ID NO: 335 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16E) or are substitutions with an amino acid present at that position in the heavy chain of one or more of the other therapeutic antibodies, for example, as identified by the alignment in FIG. 20A. In specific embodiments, the IL6 antigen binding fragment comprises a light chain comprising an amino acid sequence of SEQ ID NO: 336 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16E) or are substitutions with an amino acid present at that position in the light chain of one or more of the other therapeutic antibodies, for example, as identified by the alignment in FIG. 20B.

In certain embodiments, the anti-IL6 antigen-binding fragment transgene encodes a hyperglycosylated sirukumab Fab, comprising a heavy chain and a light chain of SEQ ID NOs: 335 and 336, respectively, with one or more of the following mutations: T114N (heavy chain), Q159N or Q159S (light chain), and/or E194N (light chain) (see FIGS. 20A (heavy chain) and 20B (light chain)).

In certain embodiments, the anti-IL6 antigen-binding fragment transgene encodes an antigen-binding fragment and comprises the nucleotide sequences encoding the six sirukumab CDRs which are underlined in the heavy and light chain variable domain sequences of FIG. 16E which are spaced between framework regions, generally human framework regions, and associated with constant domains depending upon the form of the antigen-binding molecule, as is known in the art to form the heavy and/or light chain variable domain of an anti-IL6 antibody or antigen-binding fragment thereof.

In certain embodiments, the anti-IL6 antigen-binding fragment transgene comprises the nucleotide sequences encoding the heavy and light chains of the Fab portion of olokizumab (having amino acid sequences of SEQ ID NOs. 337 and 338, respectively, see Table 5 and FIG. 16F). The nucleotide sequences may be codon optimized for expression in human cells. Nucleotide sequences may, for example, comprise the nucleotide sequences of SEQ ID NO: 349 (encoding the olokizumab heavy chain Fab portion) and SEQ ID NO: 350 (encoding the olokizumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL6-antigen binding domain has a heavy chain Fab domain of SEQ ID NO: 337 with additional hinge region sequence starting after the C-terminal valine (V), contains all or a portion of the amino acid sequence ESKYGPPCPPCPAPEFLGG (SEQ ID NO: 214), and specifically, ESKYGPPCPPCPA (SEQ ID NO: 216), ESKYGPPCPSCPA (SEQ ID NO: 217), ESKYGPPCPSCPAPEFLGGPSVFL (SEQ ID NO: 218), or ESKYGPPCPPCPAPEFLGGPSVFL (SEQ ID NO: 219) as set forth in FIG. 16F. These hinge regions may be encoded by nucleotide sequences at the 3′ end of SEQ ID NO: 349 by the hinge region encoding sequences set forth in Table 6 (SEQ ID NO: 349). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 358 (Table 7) or an IgG4 Fc domain, such as SEQ ID No. 285 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL6 antigen-binding fragment transgene encodes an IL6 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 338. In certain embodiments, the anti-IL6 antigen-binding fragment transgene encodes an IL6 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 337. In certain embodiments, the anti-IL6 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 338 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 337. In specific embodiments, the IL6 antigen binding fragment comprises a heavy chain comprising an amino acid sequence of SEQ ID NO: 337 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16F) or are substitutions with an amino acid present at that position in the heavy chain of one or more of the other therapeutic antibodies, for example, as identified by the alignment in FIG. 20A. In specific embodiments, the IL6 antigen binding fragment comprises a light chain comprising an amino acid sequence of SEQ ID NO: 338 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16F) or are substitutions with an amino acid present at that position in the light chain of one or more of the other therapeutic antibodies, for example, as identified by the alignment in FIG. 20B.

In certain embodiments, the anti-IL6 antigen-binding fragment transgene encodes a hyperglycosylated olizikumab Fab, comprising a heavy chain and a light chain of SEQ ID NOs: 337 and 338, respectively, with one or more of the following mutations: L115N (heavy chain), Q160N or Q160S (light chain), and/or E195N (light chain) (see FIGS. 20A (heavy chain) and 20B (light chain)).

In certain embodiments, the anti-IL6 antigen-binding fragment transgene encodes an antigen-binding fragment and comprises the nucleotide sequences encoding the six olokizumab CDRs which are underlined in the heavy and light chain variable domain sequences of FIG. 16F which are spaced between framework regions, generally human framework regions, and associated with constant domains depending upon the form of the antigen-binding molecule, as is known in the art to form the heavy and/or light chain variable domain of an anti-IL6 antibody or antigen-binding fragment thereof.

In certain embodiments, the anti-IL6 antigen-binding fragment transgene comprises the nucleotide sequences encoding the heavy and light chains of the Fab portion of gerilimzumab (having amino acid sequences of SEQ ID NOs. 339 and 340, respectively, see Table 5 and FIG. 16G). The nucleotide sequences may be codon optimized for expression in human cells. Nucleotide sequences may, for example, comprise the nucleotide sequences of SEQ ID NO: 351 (encoding the gerilimzumab heavy chain Fab portion) and SEQ ID NO: 352 (encoding the gerilimzumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL6-antigen binding domain has a heavy chain Fab domain of SEQ ID NO: 339 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 16G. These hinge regions may be encoded by nucleotide sequences at the 3′ end of SEQ ID NO: 351 by the hinge region encoding sequences set forth in Table 7 (SEQ ID NO: 351). In another embodiment, the transgenes comprise the amino acid sequences encoding the full length (or substantially full length) heavy and light chains of the antibody, comprising the Fc domain at the C terminus of the heavy chain, e.g. having an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL6 antigen-binding fragment transgene encodes an IL6 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 340. In certain embodiments, the anti-IL6 antigen-binding fragment transgene encodes an IL6 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 339. In certain embodiments, the anti-IL6 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 340 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 339. In specific embodiments, the IL6 antigen binding fragment comprises a heavy chain comprising an amino acid sequence of SEQ ID NO: 339 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16G) or are substitutions with an amino acid present at that position in the heavy chain of one or more of the other therapeutic antibodies, for example, as identified by the alignment in FIG. 20A. In specific embodiments, the IL6 antigen binding fragment comprises a light chain comprising an amino acid sequence of SEQ ID NO: 340 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16G) or are substitutions with an amino acid present at that position in the light chain of one or more of the other therapeutic antibodies, for example, as identified by the alignment in FIG. 20B.

In certain embodiments, the anti-IL6 antigen-binding fragment transgene encodes a hyperglycosylated gerilimzumab Fab, comprising a heavy chain and a light chain of SEQ ID NOs: 339 and 340, respectively, with one or more of the following mutations: M117N (heavy chain), and/or Q198N (light chain) (see FIGS. 20A (heavy chain) and 20B (light chain)).

In certain embodiments, the anti-IL6 antigen-binding fragment transgene encodes an antigen-binding fragment and comprises the nucleotide sequences encoding the six gerilimzumab CDRs which are underlined in the heavy and light chain variable domain sequences of FIG. 16G which are spaced between framework regions, generally human framework regions, and associated with constant domains depending upon the form of the antigen-binding molecule, as is known in the art to form the heavy and/or light chain variable domain of an anti-IL6 antibody or antigen-binding fragment thereof.

In certain embodiments, the anti-CD19 antigen-binding fragment transgene comprises the nucleotide sequences encoding the heavy and light chains of the Fab portion of inebilizumab (having amino acid sequences of SEQ ID NOs. 63 and 64, respectively, see Table 5 and FIG. 16I). The nucleotide sequences may be codon optimized for expression in human cells. Nucleotide sequences may, for example, comprise the nucleotide sequences of SEQ ID NO: 133 (encoding the inebilizumab heavy chain Fab portion) and SEQ ID NO: 134 (encoding the inebilizumab light chain Fab portion) as set forth in Table 6. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-CD19-antigen binding domain has a heavy chain Fab domain of SEQ ID NO: 63 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 16C. These hinge regions may be encoded by nucleotide sequences at the 3′ end of SEQ ID NO: 133 by the hinge region encoding sequences set forth in Table 6 (SEQ ID NO: 133). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 311 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-CD19 antigen-binding fragment transgene encodes an CD19 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 64. In certain embodiments, the anti-CD19 antigen-binding fragment transgene encodes an CD19 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 63. In certain embodiments, the anti-CD19 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 64 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 63. In specific embodiments, the CD19 antigen binding fragment comprises a heavy chain comprising an amino acid sequence of SEQ ID NO: 63 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16I) or are substitutions with an amino acid present at that position in the heavy chain of one or more of the other therapeutic antibodies, for example, as identified by the alignment in FIG. 20A. In specific embodiments, the CD19 antigen binding fragment comprises a light chain comprising an amino acid sequence of SEQ ID NO: 64 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 16I) or are substitutions with an amino acid present at that position in the light chain of one or more of the other therapeutic antibodies, for example, as identified by the alignment in FIG. 20B.

In certain embodiments, the anti-CD19 antigen-binding fragment transgene encodes a hyperglycosylated inebilizumab Fab, comprising a heavy chain and a light chain of SEQ ID NOs: 63 and 64, respectively, with one or more of the following mutations: L116N (heavy chain), Q164N or Q164S (light chain), and/or E199N (light chain) (see FIGS. 20A (heavy chain) and 20B (light chain)).

In certain embodiments, the anti-CD19 antigen-binding fragment transgene encodes an antigen-binding fragment and comprises the nucleotide sequences encoding the six inebilizumab CDRs which are underlined in the heavy and light chain variable domain sequences of FIG. 16I which are spaced between framework regions, generally human framework regions, and associated with constant domains depending upon the form of the antigen-binding molecule, as is known in the art to form the heavy and/or light chain variable domain of an anti-CD19 antibody or antigen-binding fragment thereof.

Gene Therapy Methods

Provided are methods of treating human subjects for NMO by administration of a viral vector containing a transgene encoding an anti-IL6R, anti-IL6, or anti-CD19 antibody, or antigen binding fragment thereof. The antibody may be satralizumab, sarilumab, tocilizumab, siltuximab, clazakizumab, sirukumab, olokizumab, gerilimzumab, or inebilizumab, and is, e.g., a full length, substantially full length or Fab fragment thereof, or other antigen-binding fragment thereof.

Also provided are methods of treating human subjects for non-infectious uveitis, DR, or DME by administration of a viral vector containing a transgene encoding an anti-IL6R antibody, or anti-IL6 antibody, or antigen binding fragment thereof. The antibody may be satralizumab, sarilumab, tocilizumab, siltuximab, clazakizumab, sirukumab, olokizumab, or gerilimzumab, and is, e.g., a Fab fragment thereof, or other antigen-binding fragment thereof.

In embodiments, the patient has been diagnosed with and/or has symptom(s) associated with one or more ocular disorders. Recombinant vectors used for delivering the transgene are described in Section 5.4.3. Such vectors should have a tropism for human retina-type cells and can include non-replicating rAAV, particularly those bearing an AAV8 capsid. Alternatively, vectors bearing an AAV2.7m8 or AAV9 capsid can be used for ocular indications. The recombinant vectors, such as the ones shown in FIGS. 16A-I, can be administered in any manner such that the recombinant vector enters the retina, e.g., by introducing the recombinant vector into the eye. See Section 5.5.3 for details regarding the methods of treatment.

Provided are methods of treating, inhibiting or ameliorating a detrimental immune response in human subjects by administration of a viral vector containing a transgene encoding an anti-IL6R or anti-IL6 antibody, or antigen binding fragment thereof. The antibody may be satralizumab, sarilumab, tocilizumab, siltuximab, clazakizumab, sirukumab, olokizumab, or gerilimzumab, and is, e.g., a full length, substantially full length or Fab fragment thereof, or other antigen-binding fragment thereof.

In embodiments, the patient has been diagnosed with and/or has symptom(s) associated with a detrimental immune response, such as inflammation or a cytokine storm associated with a viral or bacterial infection or is in need of therapy to manage adverse side effects, including prophylactically, such as inflammation and/or cytokine release syndrome that may be triggered by a bacterial or viral infection, or an immunotherapeutic, such as an immune-oncology therapeutic or an immune cell based therapy, such as, for example, a CAR-T cell based therapy. Viral infections include influenza, coronavirus, such as SARS-CoV-2 or other coronaviral infection, etc. Immunotherapeutics that may trigger adverse effects that may be prevented or ameliorated include BiTE single-chain antibodies, CAR-T therapeutics, such as, but not limited to tisagenlecleucel (KYMRIAH®) or axicabtagene ciloleucel (YESCARTA®) or other immunooncology therapeutics, such as anti-thymocyte globulin (ATG), the CD28 superagonist TGN1412, rituximab, obinutuzumab, alemtuzumab, brentuximab, dacetuzumab, ipilimumab, nivolumab or pembrolizumab. oxaliplatin or lenalidomide. Recombinant vectors used for delivering the transgene are described in Section 5.4.2. Such vectors should have a tropism for human muscle or liver cells and can include non-replicating rAAV, particularly those bearing an AAV8 or AAV9 capsid. The recombinant vectors, such as the ones shown in FIGS. 16A-H, can be administered in any manner such that the recombinant vector enters the liver or muscles, e.g., by introducing the recombinant vector into the bloodstream. See Section 5.5.2 for details regarding the methods of treatment.

Subjects to whom such gene therapy is administered can be those responsive to anti-IL6R, anti-IL6, or anti-CD19 therapy. In certain embodiments, the methods encompass treating patients who have been diagnosed with one or more ocular disorders, or have one or more symptoms associated therewith, and identified as responsive to treatment with an anti-IL6R, anti-IL6, or anti-CD19 antibody or considered a good candidate for therapy with an anti-IL6R, anti-IL6, or anti-CD19 antibody. In specific embodiments, the patients have previously been treated with satralizumab, sarilumab, tocilizumab, siltuximab, clazakizumab, sirukumab, olokizumab, gerilimzumab, or inebilizumab, and have been found to be responsive to satralizumab, sarilumab, tocilizumab, siltuximab, clazakizumab, sirukumab, olokizumab, gerilimzumab, or inebilizumab. In other embodiments, the patients have been previously treated with an anti-IL6R, anti-IL6, or anti-CD19 antibody. To determine responsiveness, the anti-IL6R, anti-IL6, or anti-CD19 antibody or antigen-binding fragment transgene product (e.g., produced in cell culture, bioreactors, etc.) may be administered directly to the subject.

Human Post Translationally Modified Antibodies

The production of the anti-IL6R, anti-IL6, or anti-CD19 HuPTM mAb or HuPTM Fab, should result in a “biobetter” molecule for the treatment of one of more ocular disorders accomplished via gene therapy—e.g., by administering a viral vector or other DNA expression construct encoding the anti-IL6R, anti-IL6, or anti-CD19 HuPTM Fab, subretinally, intravitreally, or suprachoroidally to human subjects (patients) diagnosed with or having one or more symptoms of non-infectious uveitis, NMO, DR, or DME, to create a permanent depot in the retina that continuously supplies the fully-human post-translationally modified, e.g., human-glycosylated, sulfated transgene product produced by transduced cells of the retina. Alternatively, the production of the anti-IL6R, anti-IL6, or anti-CD19 HuPTM mAb or HuPTM Fab, should result in a “biobetter” molecule for the treatment, inhibition or amelioration of a detrimental immune response accomplished via gene therapy—e.g., by administering a viral vector or other DNA expression construct encoding the anti-IL6R or anti-IL6, HuPTM Fab or HuPTM Mab, subcutaneously, intramuscularly or intravenously to human subjects (patients) diagnosed with or having one or more symptoms of a detrimental immune response or to manage side effects of immune therapy, to create a permanent depot in the retina that continuously supplies the fully-human post-translationally modified, e.g., human-glycosylated, sulfated transgene product produced by transduced cells of the liver and/or muscle.

In specific embodiments, the anti-IL6R HuPTM mAb or antigen-binding fragment thereof has heavy and light chains with the amino acid sequences of the heavy and light chain Fab portions of satralizumab as set forth in FIG. 16A (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N77, N161, N194, and/or N203 of the heavy chain (SEQ ID NO:59) or Q100, N158, and/or N210 of the light chain (SEQ ID NO: 60). Alternatively or in addition to, the HuPTM mAb or antigen binding-fragment thereof with the heavy and light chain variable domain sequences of satralizumab has a sulfation group at Y94, Y95, and/or Y200 of the heavy chain (SEQ ID NO: 59) and/or Y49, Y50, Y86, and/or Y87 of the light chain (SEQ ID NO: 60). In other embodiments, the anti-IL6R HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6R HuPTM mAb or antigen-binding fragment thereof has heavy and light chains with the amino acid sequences of the heavy and light chain Fab portions of sarilumab as set forth in FIG. 16B (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N54, Q108, and/or N158 of the heavy chain (SEQ ID NO:61) or Q100, N158, and/or N210 of the light chain (SEQ ID NO:62). Alternatively or in addition to, the HuPTM mAb or antigen binding-fragment thereof with the heavy and light chain variable domain sequences of sarilumab has a sulfation group at Y32, Y94, and/or Y95 of the heavy chain (SEQ ID NO:61) and/or Y86, Y87, and/or Y192 of the light chain (SEQ ID NO:62). In other embodiments, the anti-IL6R HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6R HuPTM mAb or antigen-binding fragment thereof has heavy and light chains with the amino acid sequences of the heavy and light chain Fab portions of tocilizumab as set forth in FIG. 16H (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N61, N77, and/or N161 of the heavy chain (SEQ ID NO: 341) or Q100, N158, and/or N210 of the light chain (SEQ ID NO: 342). Alternatively or in addition to, the HuPTM mAb or antigen binding-fragment thereof with the heavy and light chain variable domain sequences of satralizumab has a sulfation group at Y94 and/or Y95 of the heavy chain (SEQ ID NO: 341) and/or Y86 and/or Y87 of the light chain (SEQ ID NO: 342). In other embodiments, the anti-IL6R HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6 HuPTM mAb or antigen-binding fragment thereof has heavy and light chains with the amino acid sequences of the heavy and light chain Fab portions of siltuximab as set forth in FIG. 16C (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions Q111 and/or N161 of the heavy chain (SEQ ID NO: 331) or N60, N157, and/or N209 of the light chain (SEQ ID NO: 332). Alternatively or in addition to, the HuPTM mAb or antigen binding-fragment thereof with the heavy and light chain variable domain sequences of siltuximab has a sulfation group at Y94 and/or Y95 of the heavy chain (SEQ ID NO: 331) and/or Y85 and/or Y86 of the light chain (SEQ ID NO: 332). In other embodiments, the anti-IL6 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6 HuPTM mAb or antigen-binding fragment thereof has heavy and light chains with the amino acid sequences of the heavy and light chain Fab portions of clazakizumab as set forth in FIG. 16D (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N76, Q112, and/or N162 of the heavy chain (SEQ ID NO: 333) or N30, N161, and/or N213 of the light chain (SEQ ID NO: 334). Alternatively or in addition to, the HuPTM mAb or antigen binding-fragment thereof with the heavy and light chain variable domain sequences of clazakizumab has a sulfation group at Y93 and/or Y94 of the heavy chain (SEQ ID NO: 333) and/or Y86 and/or Y87 of the light chain (SEQ ID NO: 334). In other embodiments, the anti-IL6 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6 HuPTM mAb or antigen-binding fragment thereof has heavy and light chains with the amino acid sequences of the heavy and light chain Fab portions of sirukumab as set forth in FIG. 16E (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions Q111 and/or N161 of the heavy chain (SEQ ID NO: 335) or N157 and/or N209 of the light chain (SEQ ID NO: 336). Alternatively or in addition to, the HuPTM mAb or antigen binding-fragment thereof with the heavy and light chain variable domain sequences of sirukumab has a sulfation group at Y94 and/or Y95 of the heavy chain (SEQ ID NO: 335) and/or Y85 and/or Y86 of the light chain (SEQ ID NO: 336). In other embodiments, the anti-IL6 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6 HuPTM mAb or antigen-binding fragment thereof has heavy and light chains with the amino acid sequences of the heavy and light chain Fab portions of olokizumab as set forth in FIG. 16F (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N79, Q112, N162, and/or N204 of the heavy chain (SEQ ID NO: 337) or Q100, N158 and/or N210 of the light chain (SEQ ID NO: 338). Alternatively or in addition to, the HuPTM mAb or antigen binding-fragment thereof with the heavy and light chain variable domain sequences of olokizumab has a sulfation group at Y96 and/or Y97 of the heavy chain (SEQ ID NO: 337) and/or Y86 and/or Y87 of the light chain (SEQ ID NO: 338). In other embodiments, the anti-IL6 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6 HuPTM mAb or antigen-binding fragment thereof has heavy and light chains with the amino acid sequences of the heavy and light chain Fab portions of gerilimzumab as set forth in FIG. 16G (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N78, Q114, N164 of the heavy chain (SEQ ID NO: 339) or N71 and/or N174 of the light chain (SEQ ID NO: 340). Alternatively or in addition to, the HuPTM mAb or antigen binding-fragment thereof with the heavy and light chain variable domain sequences of gerilimzumab has a sulfation group at Y95 and/or Y96 of the heavy chain (SEQ ID NO: 339) and/or Y88 and/or Y89 of the light chain (SEQ ID NO: 340). In other embodiments, the anti-IL6 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-CD19 HuPTM mAb or antigen-binding fragment thereof has heavy and light chains with the amino acid sequences of the heavy and light chain Fab portions of inebilizumab as set forth in FIG. 16I (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N77, Q113, and/or N163 of the heavy chain (SEQ ID NO:63) or N80, N162, and/or N214 of the light chain (SEQ ID NO:64). Alternatively or in addition to, the HuPTM mAb or antigen binding-fragment thereof with the heavy and light chain variable domain sequences of inebilizumab has a sulfation group at Y94, and/or Y95 of the heavy chain (SEQ ID NO:63) and/or Y90 and/or Y91 of the light chain (SEQ ID NO:64). In other embodiments, the anti-CD19 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of or relieve one or more symptoms of one or more ocular disorders. In the case of non-infectious uveitis, efficacy may be monitored by measuring a reduction in pain, redness, and/or photophobia and/or an improvement in vision from baseline. In the case of NMO, efficacy may be monitored by measuring an improvement in vision and sensation and/or reduction of weakness or paralysis in the legs or arms, painful spasms, and/or uncontrollable vomiting and hiccups. In the case of non-infectious uveitis, efficacy may be monitored by monitoring vision acuity, redness of the eye, sensitivity to light, and/or eye pain. For example, efficacy can be monitored by assessing change in vision acuity, redness of the eye, sensitivity to light, and/or eye pain from baseline. In the case of DR and DME, efficacy may be monitored by monitoring vision acuity. For example, efficacy can be monitored by assessing change in vision acuity from baseline.

Alternatively, the goal of the gene therapy is to reduce, inhibit or ameliorate a detrimental immune response in a human subject, such as a subject suffering from a viral or bacterial infection, including infection by SARS-coV-2 or COVID19 or in need of management of side effects from immunotherapy, such as being administered an immune-oncology agent and/or a cell based immune therapy such as a CAR-T cell therapy. Symptoms of a detrimental immune response, including a cytokine release syndrome, include high fever, inflammation, severe fatigue, nausea, and may lead to tissue or organ damage, including multiple organ failure.

Combinations of delivery of the anti-IL6R, anti-IL6, or anti-CD19 HuPTM mAb or antigen-binding fragment thereof, to the liver or muscles accompanied by delivery of other available treatments are encompassed by the methods provided herein. The additional treatments may be administered before, concurrently, or subsequent to the gene therapy treatment. Available treatments for a subject with non-infectious uveitis that could be combined with the gene therapy provided herein include but are not limited to, azathioprine, methotrexate, mycophenolate mofetil, cyclosporine, cyclophosphamide, corticosteroids (local and/or systemic), and others and administration with anti-IL6R or anti-IL6, including but not limited to sarilumab, satralizumab, tocilizumab, siltuximab, clazakizumab, sirukumab, olokizumab, or gerilimzumab. Available treatments for a subject with NMO that could be combined with the gene therapy provided herein include but are not limited to, azathioprine (IMRUN®, AZASAN®), methotrexate, mycophenolate mofetil (CELLCEPT®), rituximab (RITUXAN®), corticosteroids (local and/or systemic), and others and administration with anti-IL6R, anti-IL6, or anti-CD19 agents, including but not limited to satralizumab, sarilumab, tocilizumab, siltuximab, clazakizumab, sirukumab, olokizumab, gerilimzumab, or inebilizumab. Available treatments for DR or DME that could be combined with the gene therapy provided herein include but are not limited to laser photocoagulation, photodynamic therapy with verteporfin, aflibercept, and/or intravitreal steroids, anti-VEGF agents, and others and administration with anti-IL6R agents or anti-CD19 agents, including but not limited to sarilumab, satralizumab, tocilizumab, siltuximab, clazakizumab, sirukumab, olokizumab, gerilimzumab, or inebilizumab.

### 5.3.16 Anti-Integrin (β7 Subunit) HuPTM Constructs and Formulations for Crohn's Disease and Ulcerative Colitis

Compositions and methods are described for the delivery of HuPTM mAbs and antigen-binding fragments thereof, such as HuPTM Fabs, that bind to integrin 137 subunit (ITGB7) indicated for treating inflammatory bowel disease (IBD) (e.g., UC and CD). In certain embodiments, the HuPTM mAb has the amino acid sequence of etrolizumab or an antigen binding fragment thereof. The amino acid sequence of Fab fragments of etrolizumab is provided in FIG. 17. Delivery may be accomplished via gene therapy—e.g., by administering a viral vector or other DNA expression construct encoding an ITGB7-binding HuPTM mAb (or an antigen binding fragment and/or a hyperglycosylated derivative or other derivative, thereof) to patients (human subjects) diagnosed with, or having one or more symptoms of IBD (e.g., UC or CD) to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to ITGB7 that can be administered to deliver the HuPTM mAb or antigen binding fragment in a patient. The transgene is a nucleic acid comprising the nucleotide sequences encoding an antigen binding fragment of an antibody that binds to ITGB7, such as etrolizumab, or variants thereof as detailed herein. The transgene may also encode an anti-ITGB7 antigen binding fragment that contains additional glycosylation sites (e.g., see Courtois et al.).

In certain embodiments, the anti-ITGB7 antigen-binding fragment transgene comprises the nucleotide sequences encoding the heavy and light chains of the Fab portion of etrolizumab (having amino acid sequences of SEQ ID NOs. 65 and 66, respectively, see Table 5 and FIG. 17). The nucleotide sequences may be codon optimized for expression in human cells. Nucleotide sequences may, for example, comprise the nucleotide sequences of SEQ ID NO: 135 (encoding the etrolizumab heavy chain Fab portion) and SEQ ID NO: 136 (encoding the etrolizumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-ITGB7 antigen binding domain has a heavy chain variable domain of SEQ ID NO: 65 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 17. These hinge regions may be encoded by nucleotide sequences at the 3′ end of SEQ ID NO: 135 by the hinge region encoding sequences set forth in Table 6 (SEQ ID NO: 135). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 311 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-ITGB7 antigen-binding fragment transgene encodes an ITGB7 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 66. In certain embodiments, the anti-ITGB7 antigen-binding fragment transgene encodes an ITGB7 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 65. In certain embodiments, the anti-ITGB7 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 66 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 65. In specific embodiments, the ITGB7 antigen binding fragment comprises a heavy chain comprising an amino acid sequence of SEQ ID NO: 65 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 17) or are substitutions with an amino acid present at that position in the heavy chain of one or more of the other therapeutic antibodies, for example, as identified by the alignment in FIG. 20A. In specific embodiments, the ITGB7 antigen binding fragment comprises a light chain comprising an amino acid sequence of SEQ ID NO: 66 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 17) or are substitutions with an amino acid present at that position in the light chain of one or more of the other therapeutic antibodies, for example, as identified by the alignment in FIG. 20B.

In certain embodiments, the anti-ITGB7 antigen-binding fragment transgene encodes a hyperglycosylated etrolizumab Fab, comprising a heavy chain and a light chain of SEQ ID NOs: 65 and 66, respectively, with one or more of the following mutations: L112N (heavy chain), Q160N or Q160S (light chain), and/or E195N (light chain) (see FIGS. 20A (heavy chain) and 20B (light chain)).

In certain embodiments, the anti-ITGB7 antigen-binding fragment transgene encodes an antigen-binding fragment and comprises the nucleotide sequences encoding the six etrolizumab CDRs which are underlined in the heavy and light chain variable domain sequences of FIG. 17 which are spaced between framework regions, generally human framework regions, and associated with constant domains depending upon the form of the antigen-binding molecule, as is known in the art to form the heavy and/or light chain variable domain of an anti-ITGB7 antibody or antigen-binding fragment thereof.

Gene Therapy Methods

Provided are methods of treating human subjects for IBD by administration of a viral vector containing a transgene encoding an anti-ITGB7 antibody or antigen binding fragment thereof. The antibody may be etrolizumab and is, e.g., a full length or substantially full length antibody or Fab fragment thereof, or other antigen-binding fragment thereof. In embodiments, the patient has been diagnosed with and/or has symptoms associated with one or more of IBD.

Recombinant vector used for delivering the transgene are described in Section 5.4.3. Recombinant vectors used for delivering the transgene are described in Section 5.4.2. Such vectors should have a tropism for human liver cells and can include non-replicating rAAV, particularly those bearing an AAV8 or AAV9 capsid. The recombinant vectors, such as those shown in FIG. 17, can be administered in any manner such that the recombinant vector enters the liver, e.g., by introducing the recombinant vector into the bloodstream. See Section 5.5.2 for details regarding the methods of treatment.

Subjects to whom such gene therapy is administered can be those responsive to anti-ITGB7 therapy. In certain embodiments, the methods encompass treating patients who have been diagnosed with IBD, or have one or more symptoms associated therewith, and identified as responsive to treatment with an anti-ITGB7 antibody, or considered a good candidate for therapy with an anti-ITGB7 antibody. In specific embodiments, the patients have previously been treated with etrolizumab, and have been found to be responsive to etrolizumab. To determine responsiveness, the anti-ITGB7 or antigen-binding fragment transgene product (e.g., produced in cell culture, bioreactors, etc.) may be administered directly to the subject.

Human Post Translationally Modified Antibodies

The production of the anti-ITGB7 HuPTM mAb or HuPTM Fab, should result in a “biobetter” molecule for the treatment of IBD accomplished via gene therapy—e.g., by administering a viral vector or other DNA expression construct encoding the anti-ITGB7 HuPTM Fab, subcutaneously, intramuscularly, or intravenously to human subjects (patients) diagnosed with IBD, to create a permanent depot in the muscle or liver that continuously supplies the fully-human post-translationally modified, e.g., human-glycosylated, sulfated transgene product produced by transduced cells of the muscle or liver.

The cDNA construct for the anti-ITGB7 HuPTMmAb or anti-ITGB7 HuPTM Fab should include a signal peptide that ensures proper co- and post-translational processing (glycosylation and protein sulfation) by the transduced cells of the muscle or liver. For example, the signal sequence may be MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by cells of the muscle or liver, respectively.

As an alternative, or an additional treatment to gene therapy, the anti-ITGB7 HuPTM mAb or HuPTM Fab can be produced in human cell lines by recombinant DNA technology and administered to patients diagnosed with a retinal disorder or cancer for whom therapy for IBD is considered appropriate.

In specific embodiments, the anti-ITGB7 HuPTM mAb or antigen-binding fragment thereof has heavy and light chains with the amino acid sequences of the heavy and light chain Fab portions of etrolizumab as set forth in FIG. 17 (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N60, N76, Q109, and/or N159 of the heavy chain (SEQ ID NO: 65) or Q100, N158, and/or N210 of the light chain (SEQ ID NO: 66). Alternatively or in addition to, the HuPTM mAb or antigen binding-fragment thereof with the heavy and light chain variable domain sequences of etrolizumab has a sulfation group at Y93 and/or Y94 of the heavy chain (SEQ ID NO: 65) and/or Y36, Y86, and/or Y87 of the light chain (SEQ ID NO: 66). In other embodiments, the anti-ITGB7 HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of the disease being treated or alleviate one or more symptoms thereof. With regard to CD, efficacy can be monitored by assessing Crohn's Disease Activity Index [CDAI] over the course of treatment (e.g., see Best W R et al. (1976) Gastroenterology, March; 70(3):439-44, “Development of a Crohn's disease activity index. National Cooperative Crohn's Disease Study.”). With regard to UC, efficacy can be monitored by assessing a Mayo score and an endoscopy subscore over the course of treatment (e.g., see Lobaton et al., “The Modified Mayo Endoscopic Score (MMES): A New Index for the Assessment of Extension and Severity of Endoscopic Activity in Ulcerative Colitis Patients,” J. Crohns Colitis. 2015 October: 9(10):846-52.

Combinations of delivery of the anti-ITGB7 HuPTM mAb or antigen-binding fragment thereof to the muscle or liver accompanied by delivery of other available treatments are encompassed by the methods provided herein. The additional treatments may be administered before, concurrently, or subsequent to the gene therapy treatment. Available treatments for IBD that could be combined with the gene therapy provided herein include but are not limited to nonsteroidal anti-inflammatory drugs (e.g., mesalamine, sulfasalazine), steroids (e.g., hydrocortisone, prednisone, budesonide), immunosuppressants (e.g., methotrexate, mercaptopurine, azathioprine), vitamins (e.g., iron, cholecalciferol), antibiotics (e.g., amino salicylic acid, metronidazole), other antibodies (e.g., infliximab, adalimumab) and administration with anti-ITGB7, including but not limited to etrolizumab.

### 5.3.17. Anti-Sclerostin HuPTM Constructs and Formulations for Osteoporosis

Compositions and methods are described for the delivery of HuPTM mAbs and antigen-binding fragments thereof, such as HuPTM Fabs, that bind to sclerostin (SOST) derived from anti-SOST antibody, such as romosozumab (FIG. 18), and indicated for treating osteoporosis or abnormal bone loss or weakness (e.g., treating giant cell tumor of bone, treating treatment-induced bone loss, slowing the loss of (or increasing) bone mass in breast and prostate cancer patients, preventing skeletal-related events due to bone metastasis or for decreasing bone resorption and turnover. In certain embodiments, the HuPTM mAb has the amino acid sequence of romosozumab or an antigen binding fragment thereof. The amino acid sequence of Fab fragment of this antibody is provided in FIG. 18. Delivery may be accomplished via gene therapy—e.g., by administering a viral vector or other DNA expression construct encoding an SOST-binding HuPTM mAb (or an antigen binding fragment and/or a hyperglycosylated derivative or other derivative, thereof) to patients (human subjects) diagnosed with osteoporosis or suffering bone loss to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to SOST that can be administered to deliver the HuPTM mAb or antigen binding fragment in a patient. The transgene is a nucleic acid comprising the nucleotide sequences encoding an antigen binding fragment of an antibody that binds to SOST, such as romosozumab or variants thereof as detailed herein. The transgene may also encode an anti-SOST antigen binding fragment that contains additional glycosylation sites (e.g., see Courtois et al.).

In certain embodiments, the anti-SOST antigen-binding fragment transgene comprises the nucleotide sequences encoding the heavy and light chains of the Fab portion of romosozumab (having amino acid sequences of SEQ ID NOs. 67 and 68, respectively, see Table 5 and FIG. 18). The nucleotide sequences may be codon optimized for expression in human cells. Nucleotide sequences may, for example, comprise the nucleotide sequences of SEQ ID NO: 137 (encoding the heavy chain Fab portion) and SEQ ID NO: 138 (encoding the romosozumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-SOST-antigen binding domain has a heavy chain Fab domain of SEQ ID NO: 67 with additional hinge region sequence starting after the C-terminal valine (V), contains all or a portion of the amino acid sequence contains all or a portion of the amino acid sequence ERKCCVECPPCPAPPVAG (SEQ ID NO: 220) or ERKCCVECPPCPA (SEQ ID NO: 221) as set forth in FIG. 18. These hinge regions may be encoded by nucleotide sequences at the 3′ end of SEQ ID NO: 137 by the hinge region encoding sequences set forth in Table 6 (SEQ ID NO: 137). In another embodiment, the transgenes comprise the amino acid sequences encoding the full length (or substantially full length) heavy and light chains of the antibody, comprising the Fc domain at the C terminus of the heavy chain, e.g having an amino acid sequence of SEQ ID NO: 313 (Table 7) or an IgG2 Fc domain, such as SEQ ID No. 284 or as depicted in FIG. 23, or a mutant or variant thereof. The Fc domain may be engineered for altered binding to one or more Fc receptors and/or effector function as disclosed in Section 5.1.9, infra.

In certain embodiments, the anti-SOST antigen-binding fragment transgene encodes an SOST antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 68. In certain embodiments, the anti-SOST antigen-binding fragment transgene encodes an SOST antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 67. In certain embodiments, the anti-SOST antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 68 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 67. In specific embodiments, the SOST antigen binding fragment comprises a heavy chain comprising an amino acid sequence of SEQ ID NO: 67 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 18) or are substitutions with an amino acid present at that position in the heavy chain of one or more of the other therapeutic antibodies, for example, as identified by the alignment in FIG. 20A. In specific embodiments, the SOST antigen binding fragment comprises a light chain comprising an amino acid sequence of SEQ ID NO: 68 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 18) or are substitutions with an amino acid present at that position in the light chain of one or more of the other therapeutic antibodies, for example, as identified by the alignment in FIG. 20B.

In certain embodiments, the anti-SOST antigen-binding fragment transgene encodes a hyperglycosylated romosozumab Fab, comprising a heavy chain and a light chain of SEQ ID NOs: 67 and 68, respectively, with one or more of the following mutations: T118N (heavy chain), Q160N or Q160S (light chain), and/or E195N (light chain) (see FIG. 20A (heavy chain) and B (light chain)).

In certain embodiments, the anti-SOST antigen-binding fragment transgene encodes an antigen-binding fragment and comprises the nucleotide sequences encoding the six romosozumab CDRs which are underlined in the heavy and light chain variable domain sequences of FIG. 18 which are spaced between framework regions, generally human framework regions, and associated with constant domains depending upon the form of the antigen-binding molecule, as is known in the art to form the heavy and/or light chain variable domain of an anti-SOST antibody or antigen-binding fragment thereof.

Gene Therapy Methods

Provided are methods of treating human subjects for osteoporosis or abnormal bone loss (for example, in breast or prostate cancer patients or due to bone metastases) by administration of a viral vector containing a transgene encoding an anti-SOST antibody, or antigen binding fragment thereof. The antibody may be romosozumab, and is e.g. a full length or substantially full length antibody or Fab fragment thereof, or other antigen-binding fragment thereof. In embodiments, the patient has been diagnosed with and/or has symptoms associated with osteoporosis or abnormal bone loss. Recombinant vectors used for delivering the transgene are described in Section 5.4.2. Such vectors should have a tropism for human liver or muscle cells and can include non-replicating rAAV, particularly those bearing an AAV8 or AAV9 capsid. The recombinant vector, such as shown in FIG. 18, can be administered in any manner such that the recombinant vector enters the liver or muscle tissue, e.g., by introducing the recombinant vector into the bloodstream. See Section 5.5.2 for details regarding the methods of treatment.

Subjects to whom such gene therapy is administered can be those responsive to anti-SOST therapy. In certain embodiments, the methods encompass treating patients who have been diagnosed with osteoporosis or abnormal bone loss, or have one or more symptoms associated therewith, and identified as responsive to treatment with an anti-SOST antibody or considered a good candidate for therapy with an anti-SOST antibody. In specific embodiments, the patients have previously been treated with romosozumab, and have been found to be responsive to romosozumab. To determine responsiveness, the anti-SOST antibody or antigen-binding fragment transgene product (e.g., produced in cell culture, bioreactors, etc.) may be administered directly to the subject.

Human Post Translationally Modified Antibodies

The production of the anti-SOST HuPTM mAb or HuPTM Fab, should result in a “biobetter” molecule for the treatment of osteoporosis or bone loss accomplished via gene therapy—e.g., by administering a viral vector or other DNA expression construct encoding the anti-SOST HuPTM Fab, intravenously to human subjects (patients) diagnosed with or having one or more symptoms of osteoporosis or bone loss, to create a permanent depot in the liver or muscle tissue that continuously supplies the fully-human post-translationally modified, e.g., human-glycosylated, sulfated transgene product produced by transduced liver or muscle cells.

The cDNA construct for the anti-SOST HuPTMmAb or anti-SOST HuPTM Fab should include a signal peptide that ensures proper co- and post-translational processing (glycosylation and protein sulfation) by the transduced liver or muscle cells. For example, the signal sequence may be MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

As an alternative, or an additional treatment to gene therapy, the anti-SOST HuPTM mAb or HuPTM Fab can be produced in human cell lines by recombinant DNA technology, and administered to patients diagnosed with osteoporosis or bone loss, or for whom therapy for osteoporosis or bone loss is considered appropriate.

In specific embodiments, the anti-SOST HuPTM mAb or antigen-binding fragment thereof has heavy and light chains with the amino acid sequences of the heavy and light chain Fab portions of romosozumab as set forth in FIG. 18 (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions Q115, N165, N198, and/or N207 of the heavy chain (SEQ ID NO:67) or N158 and/or N210 of the light chain (SEQ ID NO:68). Alternatively or in addition to, the HuPTM mAb or antigen binding-fragment thereof with the heavy and light chain variable domain sequences of romosozumab has a sulfation group at Y94 and/or Y204 of the heavy chain (SEQ ID NO: 67) and/or Y86 and/or Y87 of the light chain (SEQ ID NO: 68). In other embodiments, the anti-SOST HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of osteoporosis or bone loss. Efficacy may be monitored by evaluating bone tissue or skeletal events or the lack of skeletal events. For example, with regard to osteoporosis, efficacy can be monitored by a bone mineral content assessment, assessment of radiographs for vertebral fractures, or diagnostic imaging for clinical fractures confirmation.

Combinations of delivery of the anti-SOST HuPTM mAb or antigen-binding fragment thereof, to the liver or muscles accompanied by delivery of other available treatments are encompassed by the methods provided herein. The additional treatments may be administered before, concurrently, or subsequent to the gene therapy treatment. Available treatments for osteoporosis or bone loss that could be combined with the gene therapy provided herein include but are not limited to bisphosphonates (e.g., zoledronic acid), parathyroid hormone (e.g., teriparatide [PTH 1-34] and/or full-length PTH 1-84), calcium, vitamin D, anti-RANKL agents, and chemotherapy, cryotherapy, or radiotherapy in patients diagnosed with cancer, and administration with anti-SOST agents, including but not limited to romosozumab.

### 5.3.18. Anti-pKal HuPTM Constructs and Formulations for Angioedema and Diabetic Retinopathy

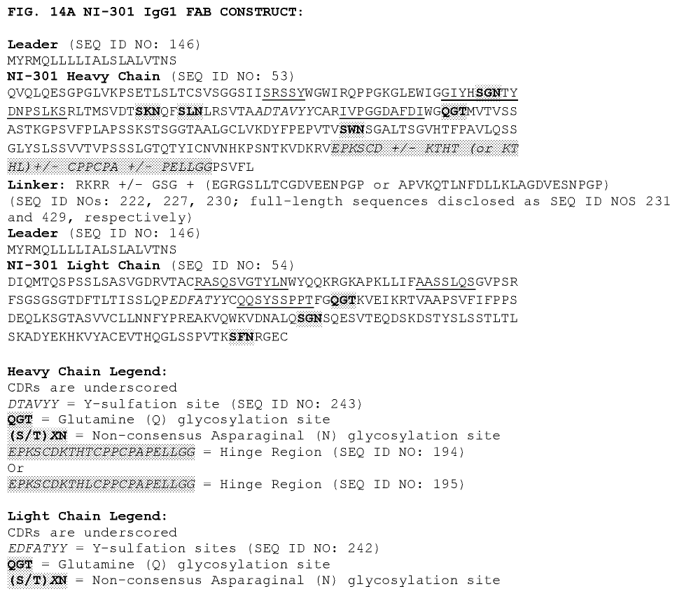

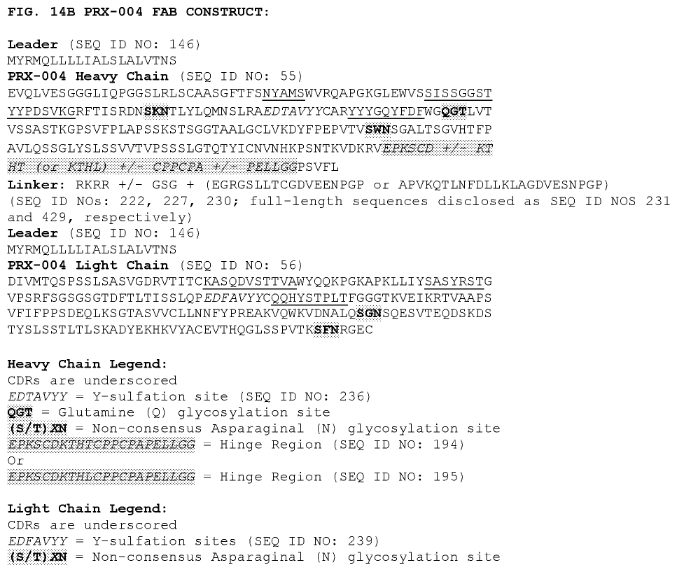

Compositions and methods are described for the delivery of HuPTM mAbs and antigen-binding fragments thereof, such as HuPTM Fabs, that bind to kallikrein (pKal), derived from an anti-pKal antibody and indicated for treating angioedema, such as hereditary angioedema. In other embodiments, compositions and methods are provided for treating diabetic retinopathy and diabetic macular edema. In certain embodiments, the HuPTM mAb has the amino acid sequence of lanadelumab or an antigen binding fragment thereof. The amino acid sequence of Fab fragment of this antibody is provided in FIG. 19. Delivery may be accomplished via gene therapy—e.g., by administering a viral vector or other DNA expression construct encoding an pKal-binding HuPTM mAb (or an antigen binding fragment and/or a hyperglycosylated derivative or other derivative, thereof) to patients (human subjects) diagnosed with angioedema or diabetic retinopathy and diabetic macular edema to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to pKal that can be administered to deliver the HuPTM mAb or antigen binding fragment in a patient. The transgene is a nucleic acid comprising the nucleotide sequences encoding an antigen binding fragment of an antibody that binds to pKal, such as lanadelumab or variants thereof as detailed herein. The transgene may also encode an anti-pKal antigen binding fragment that contains additional glycosylation sites (e.g., see Courtois et al.).

In certain embodiments, the anti-pKal antigen-binding fragment transgene comprises the nucleotide sequences encoding the heavy and light chains of the Fab portion of lanadelumab (having amino acid sequences of SEQ ID NOs. 69 and 70, respectively, see Table 5 and FIG. 19). The nucleotide sequences may be codon optimized for expression in human cells. Nucleotide sequences may, for example, comprise the nucleotide sequences of SEQ ID NO: 139 (encoding the lanadelumab heavy chain Fab portion) and SEQ ID NO: 140 (encoding the lanadelumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively. For diabetic retinopathy and diabetic macular edema indications, the signal sequence may direct expression and secretion in human ocular or retinal cells, for example, one of the signal sequences in Table 5.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-pKal-antigen binding domain has a heavy chain Fab domain of SEQ ID NO: 69 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 19. These hinge regions may be encoded by nucleotide sequences at the 3′ end of SEQ ID NO: 139 by the hinge region encoding sequences set forth in Table 6 (SEQ ID NO: 139). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 314 (Table 7) or an IgG1 Fc domain, such as SEQ ID No. 283 or as depicted in FIG. 23, or a mutant or variant thereof. The Fc domain may be engineered for altered binding to one or more Fc receptors and/or effector function as disclosed in Section 5.1.9, infra.

In specific embodiments, provided are constructs encoding a full length lanadelumab, including the Fc domain, particularly nucleotide sequence L01, L02 or L03 (SEQ ID Nos: 141, 286 or 287, respectively) as described in Example 36 and Table 8, herein, which are codon optimized and, in the case of L02 and L03 depleted for CpG dimers. The transgene may also comprises a nucleotide sequence that encodes a signal peptide MYRMQLLLLIALSLALVTNS (SEQ ID NO: 146; for example at the N-terminal of the heavy and/or the light chain) which may be encoded by the nucleotide sequence of SEQ ID NO: 422. The nucleotide sequences encoding the light chain and heavy chain may be separated by a Furin-2A linker (SEQ ID NOs: 231) to create a bicistronic vector. Alternatively, the nucleotide sequences of the light chain and heavy chain are separated by a Furin-T2A linker, such as SEQ ID NO: 429, and as encoded by SEQ ID NO: 424. Expression of the lanadelumab may be directed by a constitutive or a tissue specific promoter. In certain embodiments, the transgene contains a CAG promoter (SEQ ID NO: 411) or a TBG (SEQ ID NO; 423) promoter. Alternatively, the promoter may be a tissue specific promoter (or regulatory sequence including promoter and enhancer elements) such as the APOE.hAAT regulatory sequence (SEQ ID NO: 412), LSPX1 (SEQ ID NO: 315), LSPX3, LTP1 (SEQ ID NO; 317) or LMTP6 (SEQ ID NO: 320) promoter or CK8 (SEQ ID NO: 413) promoter. See FIG. 24A for a schematic showing the genomic configuration. The transgenes may contain elements provided in Table 1. Exemplary transgenes encoding full length lanadelumab are provided in Table 8 and include CAG.LAN.F2A (SEQ ID NO: 436); CAG.LAN.T2A (SEQ ID NO; 437); TBG.LAN.T2A (SEQ ID NO; 438); APOE.hAAT.LAN.T2A (SEQ ID NO: 439); LSPX1.LAN.T2A (SEQ ID NO; 440); LSPX2.LAN.T2A (SEQ ID NO: 441); LTP1.LAN.T2A (SEQ ID NO: 442); and LMTP6.LAN.T2A (SEQ ID NO: 443). ITR sequences are added to the 5′ and 3; ends of the constructs to generate the genomes. The transgenes may be packaged into AAV, particularly AAV8.

In certain embodiments, the anti-pKal antigen-binding fragment transgene encodes an pKal antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 70. In certain embodiments, the anti-pKal antigen-binding fragment transgene encodes an pKal antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 69. In certain embodiments, the anti-pKal antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 70 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 69. In specific embodiments, the pKal antigen binding fragment comprises a heavy chain comprising an amino acid sequence of SEQ ID NO: 69 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 19) or are substitutions with an amino acid present at that position in the heavy chain of one or more of the other therapeutic antibodies, for example, as identified by the alignment in FIG. 20A. In specific embodiments, the pKal antigen binding fragment comprises a light chain comprising an amino acid sequence of SEQ ID NO: 70 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 19) or are substitutions with an amino acid present at that position in the light chain of one or more of the other therapeutic antibodies, for example, as identified by the alignment in FIG. 20B.

In certain embodiments, the anti-pKal antigen-binding fragment transgene encodes a hyperglycosylated lanadelumab Fab, comprising a heavy chain and a light chain of SEQ ID NOs: 69 and 70, respectively, with one or more of the following mutations: M117N (heavy chain) and/or Q159N, Q159S, and/or E194N (light chain) (see FIGS. 20A (heavy chain) and 20B (light chain)).

In certain embodiments, the anti-pKal antigen-binding fragment transgene encodes an antigen-binding fragment and comprises the nucleotide sequences encoding the six lanadelumab CDRs which are underlined in the heavy and light chain variable domain sequences of FIG. 19 which are spaced between framework regions, generally human framework regions, and associated with constant domains depending upon the form of the antigen-binding molecule, as is known in the art to form the heavy and/or light chain variable domain of an anti-pKal antibody or antigen-binding fragment thereof.

Gene Therapy Methods

Provided are methods of treating human subjects for angioedema by administration of a viral vector containing a transgene encoding an anti-pKal antibody, or antigen binding fragment thereof. The antibody may be lanadelumab and is, e.g., a full length or substantially full length antibody or Fab fragment thereof, or other antigen-binding fragment thereof. In embodiments, the patient has been diagnosed with and/or has symptoms associated with angioedema. Recombinant vectors used for delivering the transgene are described in Section 5.4.2 and exemplary transgenes are provided above. Such vectors should have a tropism for human liver or muscle cells and can include non-replicating rAAV, particularly those bearing an AAV8 or AAV9 capsid, preferably AAV8. The recombinant vectors, such as shown in FIG. 19, can be administered in any manner such that the recombinant vector enters the liver or muscle tissue, e.g., by introducing the recombinant vector into the bloodstream, for example by intravenous or intramuscular administration. See Section 5.5.2 for details regarding the methods of treatment.

Provided are methods of treating human subjects for diabetic retinopathy or diabetic macular edema by administration of a viral vector containing a transgene encoding an anti-pKal antibody, or antigen binding fragment thereof. See also, Section 5.3.8 above, also. The antibody may be lanadelumab and is, e.g., a full length or substantially full length antibody or Fab fragment thereof, or other antigen-binding fragment thereof. In embodiments, the patient has been diagnosed with and/or has symptoms associated with diabetic retinopathy or diabetic macular edema. Recombinant vectors used for delivering the transgene are described in Section 5.4.3 and exemplary transgenes are provided above. Such vectors should have a tropism for human retinal cells and can include non-replicating rAAV, particularly those bearing an AAV8 or AAV9 capsid. The recombinant vectors, such as shown in FIG. 19, can be administered in any manner such that the recombinant vector enters the retinal tissue, for example as disclosed in Section 5.5.3. In particular embodiments, the transgene is CAG.LAN.F2A (SEQ ID NO: 436); CAG.LAN.T2A (SEQ ID NO; 437); TBG.LAN.T2A (SEQ ID NO; 438); APOE.hAAT.LAN.T2A (SEQ ID NO: 439); LSPX1.LAN.T2A (SEQ ID NO; 440); LSPX2.LAN.T2A (SEQ ID NO: 441); LTP1.LAN.T2A (SEQ ID NO: 442); or LMTP6.LAN.T2A (SEQ ID NO: 443) in an AAV8 vector.

See Section 5.5.2 and 5.5.4 for details regarding the methods of treatment.

Examples 51, 53, 54 and 56 herein provide results of serum levels of lanadelumab in mice and rats administered AAV vectors encoding full length lanadelumab to assess different promoters and other regulatory elements, linkers, AAV types, modes of administration, etc. Such results inform dosage of a recombinant AAV vector encoding lanadelumab to achieve serum levels, particularly, steady state serum levels, sufficient for therapeutic efficacy. Steady state serum levels of sufficient therapeutic efficacy may be determined through clinical studies, for example, as provided in the prescribing information for lanadelumab (see TAKHZYRO® Prescribing Information). In particular embodiments, the AAV8 lanadelumab vector is administered to a patient in need thereof, for example, a patient diagnosed with or suffering from HAE, at a dosage (vector genomes) sufficient for to expression of therapeutically effective levels of lanadelumab in the patient serum. In specific embodiments, the administration results in Cₘₐₓ of 9 μg/ml to 35 μg/ml, including between 12 μg/ml to 25 μg/ml, or between 20 μg/ml and 35 μg/ml; and a Cₘᵢₙ of 4 μg/ml to 25 μg/ml or a Cₘᵢₙ greater than 20 μg/ml, but in certain embodiments less than 200 μg/ml or 500 μg/ml. The serum or plasma concentration is preferably achieved as a steady state concentration, for example, maintaining serum or plasma levels within the Cₘₐₓ and Cₘᵢₙ for at least 1 month, 2 months, 3 months, or greater than 3 months, or 1 year. In specific embodiments, administration of the AAV vector results in steady state lanadelumab plasma concentration of 5 μg/ml to 30 μg/ml or 10 μg/ml to 20 μg/ml; or 15 μg/ml to 30 μg/ml or greater than 20 μg/ml, but in certain embodiments less than 200 μg/ml or 500 mg/ml. In particular embodiments, the the lanadelumab antibody secreted into the plasma exhibits greater a greater than at least 40%, 45%, 50%, 55%, 60%, 65% or 70 reduction in pKal activity as measured by a kinetic enzymatic functional assay, for example, the assay described in Example 57. In certain embodiments, the activity of the lanadelumab antibody is measured at 2 weeks, 3 weeks, 4 weeks, 5 weeks, 6 weeks, 7 weeks, 8 weeks, 9 weeks, 10 weeks, 11 weeks or 12 weeks after administration of the AAV vector. The methods of treatment provided herein reduce the incidence or severity of angioedema occurrences or attacks. In particular embodiments, the angioedema occurs in the skin, the gastrointestinal tract or the upper airway.

Subjects to whom such gene therapy is administered can be those responsive to anti-pKal therapy. In certain embodiments, the methods encompass treating patients who have been diagnosed with angioedema or diabetic retinopathy, or have one or more symptoms associated therewith, and identified as responsive to treatment with an anti-pKal antibody or considered a good candidate for therapy with an anti-pKal antibody. In specific embodiments, the patients have previously been treated with lanadelumab, and have been found to be responsive to lanadelumab. To determine responsiveness, the anti-pKal antibody or antigen-binding fragment transgene product (e.g., produced in cell culture, bioreactors, etc.) may be administered directly to the subject.

Human Post Translationally Modified Antibodies

The production of the anti-pKal HuPTM mAb or HuPTM Fab, should result in a “biobetter” molecule for the treatment of angioedema accomplished via gene therapy—e.g., by administering a viral vector or other DNA expression construct encoding the anti-pKal HuPTM Fab, intravenously to human subjects (patients) diagnosed with or having one or more symptoms of angioedema, to create a permanent depot in the liver or muscle tissue that continuously supplies the fully-human post-translationally modified, e.g., human-glycosylated, sulfated transgene product produced by transduced liver or muscle cells.

In specific embodiments, the anti-pKal HuPTM mAb or antigen-binding fragment thereof has heavy and light chains with the amino acid sequences of the heavy and light chain Fab portions of lanadelumab as set forth in FIG. 19 (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N77, Q114 and/or N164 of the heavy chain (SEQ ID NO: 69) or Q99, N157, and/or N209 of the light chain (SEQ ID NO: 70). Alternatively or in addition to, the HuPTM mAb or antigen binding-fragment thereof with the heavy and light chain variable domain sequences of lanadelumab has a sulfation group at Y94 and/or Y95 of the heavy chain (SEQ ID NO: 69) and/or Y86 and/or Y87 of the light chain (SEQ ID NO: 70). In other embodiments, the anti-pKal HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab (or a hyperglycosylated derivative of either) is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of angioedema, reduce the levels of pain or discomfort for the patient, or reduce levels of autoreactive B cells and immunoglobulin producing plasma cells. Efficacy may be monitored by scoring the function, symptoms, or degree of inflammation in the affected tissue or area of the body, e.g., such as the skin, joints, kidneys, lungs, blood cells, heart, and brain. For example, efficacy can be monitored by assessing changes in attack severity or frequency.

Combinations of delivery of the anti-pKal HuPTM mAb or antigen-binding fragment thereof, to the liver or muscle accompanied by delivery of other available treatments are encompassed by the methods provided herein. The additional treatments may be administered before, concurrently, or subsequent to the gene therapy treatment. Available treatments for angioedema that could be combined with the gene therapy provided herein include but are not limited to danazol, bradykinin receptor antagonist (e.g., icatibant), plasma kallikrein inhibitor (e.g., ecallantide), C1 esterase inhibitor, conestat alfa, anti-fibrinolytic agents (e.g., tranexamic acid), omalizumab, and fresh frozen plasma transfusions, antihistamines, and corticosteroids and administration with anti-pKal agents, including but not limited to lanadelumab.

### 5.3.19. Anti-IL and IL-Receptor and Other Targets HuPTM Constructs and Formulations for Autoimmune, Respiratory, and Allergic Diseases

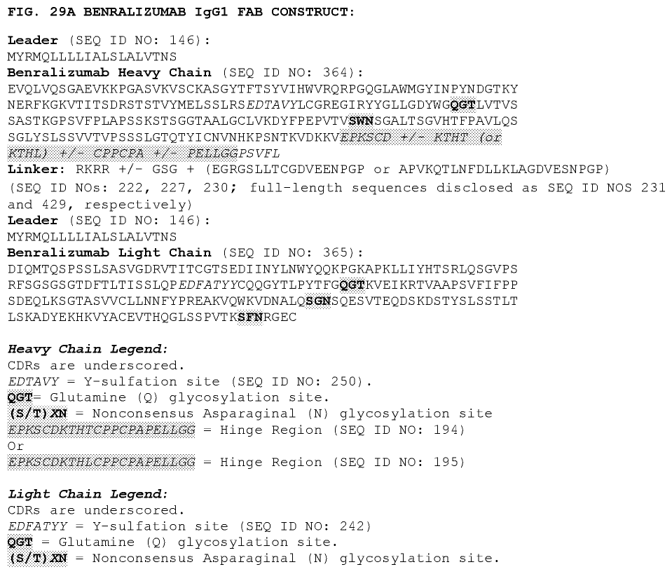

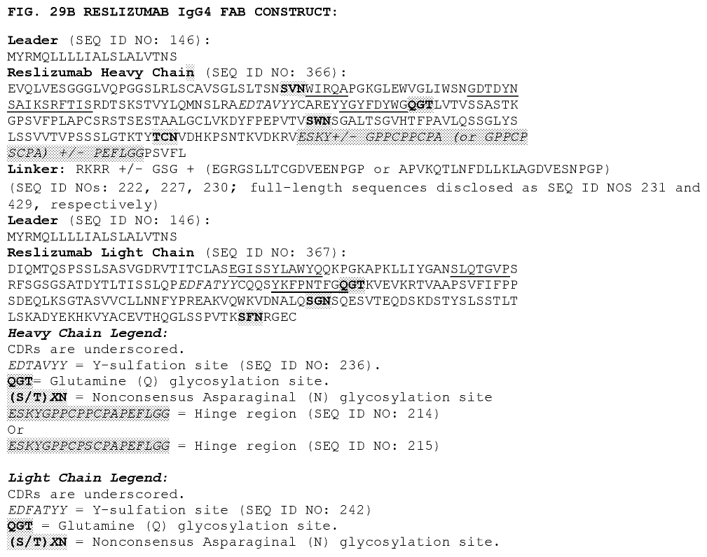

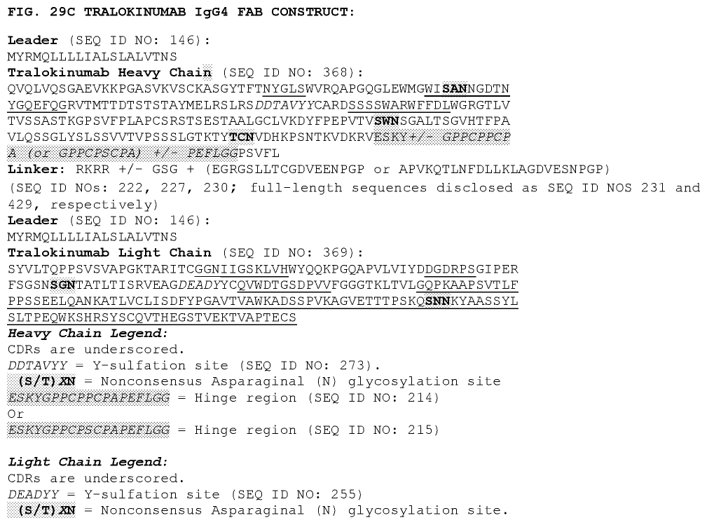

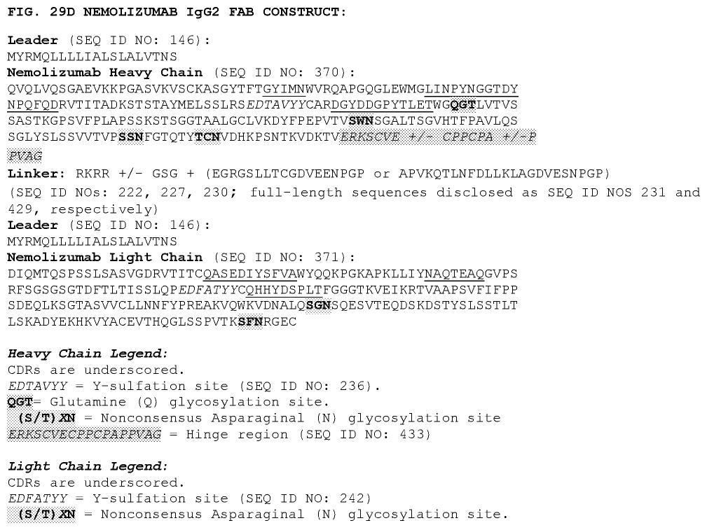

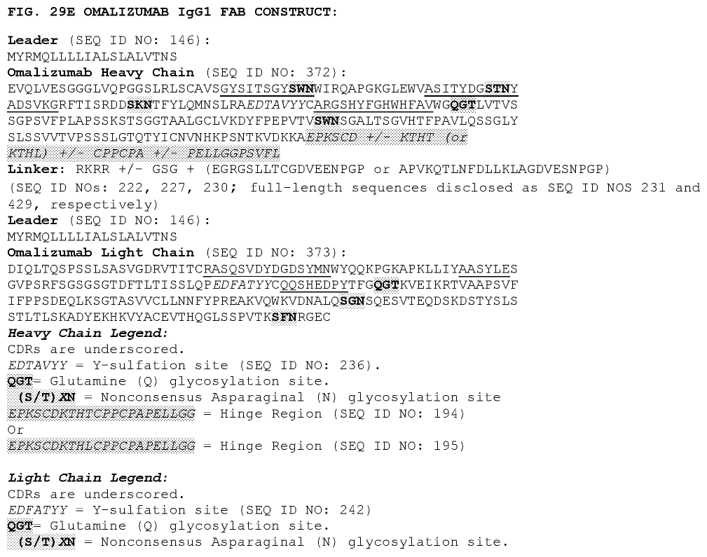

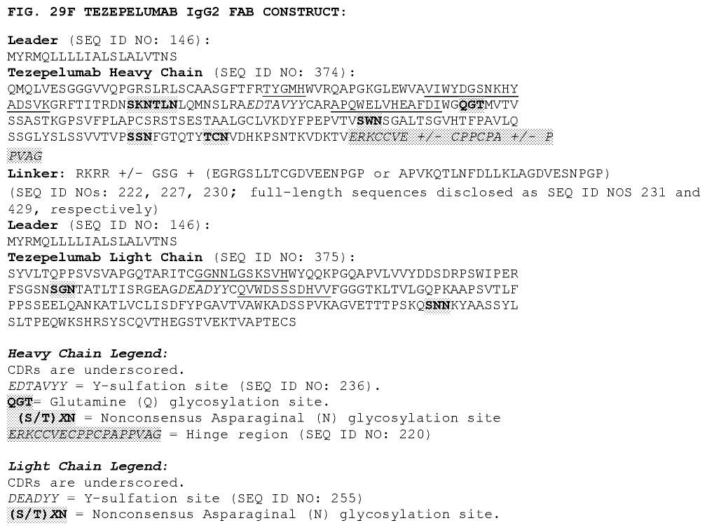

Compositions and methods are described for the delivery of HuPTM mAbs and antigen-binding fragments thereof, such as HuPTM Fabs, that bind to interleukins (IL), interleukin receptors (ILR) (e.g., IL31RA, IL13, IL5, or IL-5), immunoglobin E (IgE), or thymic stromal lymphopoietin (TSLP), derived from anti-ILs, anti-ILRs, anti-IgE, or anti-TSLP indicated for treating one or more autoimmune-related disorders, respiratory diseases, and allergic diseases, such as atopic dermatitis, chronic idiopathic urticaria, asthma, eosinophilic asthma, or chronic obstructive pulmonary disease (COPD) (collectively referred to hereinafter as “subject AI-Ds”). In particular embodiments, the HuPTM mAb has the amino acid sequence of benralizumab, reslizumab, tralokinumab, nemolizumab, omalizumab, or tezepelumab or an antigen binding fragment of one of the foregoing. The amino acid sequences of Fab fragments of these antibodies are provided in FIGS. 29A to 29F, respectively. Delivery may be accomplished via gene therapy—e.g., by administering a viral vector or other DNA expression construct encoding an IL/ILR-binding, IgE-binding, or TSLP-binding HuPTM mAb (or an antigen binding fragment and/or a hyperglycosylated derivative or other derivative, thereof) to patients (human subjects) diagnosed with, or having one or more symptoms of atopic dermatitis, chronic idiopathic urticaria, asthma, eosphinophilic asthma, or COPD to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to IL/ILR, IgE, or TSLP that can be administered to deliver the HuPTM mAb or antigen binding fragment in a patient. The transgene is a nucleic acid comprising the nucleotide sequences encoding an antigen binding fragment of an antibody that binds to IL/ILR, such as benralizumab, reslizumab, tralokinumab, nemolizumab; IgE, such as omalizumab; or TSLP, such as tezepelumab, or variants thereof as detailed herein. The transgene may also encode an anti-IL/ILR, anti-IgE, or anti-TSLP antigen binding fragment that contains additional glycosylation sites (e.g., see Courtois et al.).

In certain embodiments, the anti-IL5 antigen-binding fragment transgene comprises the nucleotide sequences encoding the heavy and light chains of the Fab portion of benralizumab (having amino acid sequences of SEQ ID NOs. 364 and 365, respectively, see Table 5 and FIG. 29A). The nucleotide sequences may be codon optimized for expression in human cells. Nucleotide sequences may, for example, comprise the nucleotide sequences of SEQ ID NO: 380 (encoding the benralizumab heavy chain Fab portion) and SEQ ID NO: 381 (encoding the benralizumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL5-antigen binding domain has a heavy chain Fab domain of SEQ ID NO: 364 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 29A. These hinge regions may be encoded by nucleotide sequences at the 3′ end of SEQ ID NO: 380 by the hinge region encoding sequences set forth in Table 6 (SEQ ID NO: 380). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 394 (Table 7) or an IgG1 Fc domain, such as SEQ ID NO: 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL5 antigen-binding fragment transgene encodes an IL5 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 365. In certain embodiments, the anti-IL5 antigen-binding fragment transgene encodes an IL5 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 364. In certain embodiments, the anti-IL5 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 365 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 364. In specific embodiments, the IL5 antigen binding fragment comprises a heavy chain comprising an amino acid sequence of SEQ ID NO: 364 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29A) or are substitutions with an amino acid present at that position in the heavy chain of one or more of the other therapeutic antibodies, for example, as identified by the alignment in FIG. 20A. In specific embodiments, the IL5 antigen binding fragment comprises a light chain comprising an amino acid sequence of SEQ ID NO: 365 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29A) or are substitutions with an amino acid present at that position in the light chain of one or more of the other therapeutic antibodies, for example, as identified by the alignment in FIG. 20B.

In certain embodiments, the anti-IL5 antigen-binding fragment transgene encodes a hyperglycosylated benralizumab Fab, comprising a heavy chain and a light chain of SEQ ID NOs: 364 and 365, respectively, with one or more of the following mutations: L116N (heavy chain) and/or Q160N, Q160S , and/or E195N (light chain) (see FIGS. 20A (heavy chain) and 20B (light chain)).

In certain embodiments, the anti-IL5 antigen-binding fragment transgene encodes an antigen-binding fragment and comprises the nucleotide sequences encoding the six benralizumab CDRs which are underlined in the heavy and light chain variable domain sequences of FIG. 29A which are spaced between framework regions, generally human framework regions, and associated with constant domains depending upon the form of the antigen-binding molecule, as is known in the art to form the heavy and/or light chain variable domain of an anti-IL5 antibody or antigen-binding fragment thereof.

In certain embodiments, the anti-IL5R antigen-binding fragment transgene comprises the nucleotide sequences encoding the heavy and light chains of the Fab portion of reslizumab (having amino acid sequences of SEQ ID NOs. 366 and 367, respectively, see Table 4 and FIG. 29B). The nucleotide sequences may be codon optimized for expression in human cells. Nucleotide sequences may, for example, comprise the nucleotide sequences of SEQ ID NO: 382 (encoding the reslizumab heavy chain Fab portion) and SEQ ID NO: 383 (encoding the reslizumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL5R-antigen binding domain has a heavy chain Fab domain of SEQ ID NO: 366 with additional hinge region sequence starting after the C-terminal valine (V), contains all or a portion of the amino acid sequence ESKYGPPCPPCPAPEFLGG (SEQ ID NO: 214), and specifically, ESKYGPPCPPCPA (SEQ ID NO: 216), ESKYGPPCPSCPA (SEQ ID NO: 217), ESKYGPPCPSCPAPEFLGGPSVFL (SEQ ID NO: 218), or ESKYGPPCPPCPAPEFLGGPSVFL (SEQ ID NO: 219) as set forth in FIG. 29B. These hinge regions may be encoded by nucleotide sequences at the 3′ end of SEQ ID NO: 382 by the hinge region encoding sequences set forth in Table 6 (SEQ ID NO: 382). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 395 (Table 7) or an IgG4 Fc domain, such as SEQ ID NO: 285 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL5R antigen-binding fragment transgene encodes an IL5R antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 367. In certain embodiments, the anti-IL5R antigen-binding fragment transgene encodes an IL5R antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 366. In certain embodiments, the anti-IL5R antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 367 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 366. In specific embodiments, the IL5R antigen binding fragment comprises a heavy chain comprising an amino acid sequence of SEQ ID NO: 366 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29B) or are substitutions with an amino acid present at that position in the heavy chain of one or more of the other therapeutic antibodies, for example, as identified by the alignment in FIG. 20A. In specific embodiments, the IL5R antigen binding fragment comprises a light chain comprising an amino acid sequence of SEQ ID NO: 367 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29B) or are substitutions with an amino acid present at that position in the light chain of one or more of the other therapeutic antibodies, for example, as identified by the alignment in FIG. 20B.

In certain embodiments, the anti-IL5R antigen-binding fragment transgene encodes a hyperglycosylated reslizumab Fab, comprising a heavy chain and a light chain of SEQ ID NOs: 366 and 367, respectively, with one or more of the following mutations: L111N (heavy chain) and/or Q160N, Q160S, and/or E195N (light chain) (see FIGS. 20A (heavy chain) and 20B (light chain)).

In certain embodiments, the anti-IL5R antigen-binding fragment transgene encodes an antigen-binding fragment and comprises the nucleotide sequences encoding the six reslizumab CDRs which are underlined in the heavy and light chain variable domain sequences of FIG. 29B which are spaced between framework regions, generally human framework regions, and associated with constant domains depending upon the form of the antigen-binding molecule, as is known in the art to form the heavy and/or light chain variable domain of an anti-IL5R antibody or antigen-binding fragment thereof.

In certain embodiments, the anti-IL13 antigen-binding fragment transgene comprises the nucleotide sequences encoding the heavy and light chains of the Fab portion of tralokinumab (having amino acid sequences of SEQ ID NOs. 368 and 369, respectively, see Table 5 and FIG. 29C). The nucleotide sequences may be codon optimized for expression in human cells. Nucleotide sequences may, for example, comprise the nucleotide sequences of SEQ ID NO: 384 (encoding the tralokinumab heavy chain Fab portion) and SEQ ID NO: 385 (encoding the tralokinumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL13-antigen binding domain has a heavy chain Fab domain of SEQ ID NO: 368 with additional hinge region sequence starting after the C-terminal valine (V), contains all or a portion of the amino acid sequence ESKYGPPCPPCPAPEFLGG (SEQ ID NO: 214), and specifically, ESKYGPPCPPCPA (SEQ ID NO: 216), ESKYGPPCPSCPA (SEQ ID NO: 217), ESKYGPPCPSCPAPEFLGGPSVFL (SEQ ID NO: 218), or ESKYGPPCPPCPAPEFLGGPSVFL (SEQ ID NO: 219) as set forth in FIG. 29C. These hinge regions may be encoded by nucleotide sequences at the 3′ end of SEQ ID NO: 384 by the hinge region encoding sequences set forth in Table 6 (SEQ ID NO: 384). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 396 (Table 7) or an IgG4 Fc domain, such as SEQ ID NO: 285 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL13 antigen-binding fragment transgene encodes an IL13 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 369. In certain embodiments, the anti-IL13 antigen-binding fragment transgene encodes an IL13 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 368. In certain embodiments, the anti-IL13 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 369 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 368. In specific embodiments, the IL13 antigen binding fragment comprises a heavy chain comprising an amino acid sequence of SEQ ID NO: 368 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29C) or are substitutions with an amino acid present at that position in the heavy chain of one or more of the other therapeutic antibodies, for example, as identified by the alignment in FIG. 20A. In specific embodiments, the IL13 antigen binding fragment comprises a light chain comprising an amino acid sequence of SEQ ID NO: 369 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29C) or are substitutions with an amino acid present at that position in the light chain of one or more of the other therapeutic antibodies, for example, as identified by the alignment in FIG. 20B.

In certain embodiments, the anti-IL13 antigen-binding fragment transgene encodes a hyperglycosylated tralokinumab Fab, comprising a heavy chain and a light chain of SEQ ID NOs: 368 and 369, respectively, with one or more of the following mutations: L117N (heavy chain) and/or Q196N (light chain) (see FIGS. 20A (heavy chain) and 20B (light chain)).

In certain embodiments, the anti-IL13 antigen-binding fragment transgene encodes an antigen-binding fragment and comprises the nucleotide sequences encoding the six tralokinumab CDRs which are underlined in the heavy and light chain variable domain sequences of FIG. 29C which are spaced between framework regions, generally human framework regions, and associated with constant domains depending upon the form of the antigen-binding molecule, as is known in the art to form the heavy and/or light chain variable domain of an anti-IL13 antibody or antigen-binding fragment thereof.

In certain embodiments, the anti-IL31RA antigen-binding fragment transgene comprises the nucleotide sequences encoding the heavy and light chains of the Fab portion of nemolizumab (having amino acid sequences of SEQ ID NOs. 370 and 371, respectively, see Table 5 and FIG. 29D). The nucleotide sequences may be codon optimized for expression in human cells. Nucleotide sequences may, for example, comprise the nucleotide sequences of SEQ ID NO: 386 (encoding the nemolizumab heavy chain Fab portion) and SEQ ID NO: 387 (encoding the nemolizumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IL31RA-antigen binding domain has a heavy chain Fab domain of SEQ ID NO: 370 with additional hinge region sequence starting after the C-terminal valine (V), contains all or a portion of the amino acid sequence ERKSCVECPPCPAPPVAG (SEQ ID NO: 433) or ERKSCVECPPCPA (SEQ ID NO: 434) as set forth in FIG. 29D. These hinge regions may be encoded by nucleotide sequences at the 3′ end of SEQ ID NO: 386 by the hinge region encoding sequences set forth in Table 6 (SEQ ID NO: 386). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 397 (Table 7) or an IgG2 Fc domain, such as SEQ ID NO: 284 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IL31RA antigen-binding fragment transgene encodes an IL31RA antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 371. In certain embodiments, the anti-IL31RA antigen-binding fragment transgene encodes an IL31RA antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 370. In certain embodiments, the anti-IL31RA antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 371 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 370. In specific embodiments, the IL31RA antigen binding fragment comprises a heavy chain comprising an amino acid sequence of SEQ ID NO: 370 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29D) or are substitutions with an amino acid present at that position in the heavy chain of one or more of the other therapeutic antibodies, for example, as identified by the alignment in FIG. 20A. In specific embodiments, the IL31RA antigen binding fragment comprises a light chain comprising an amino acid sequence of SEQ ID NO: 371 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29D) or are substitutions with an amino acid present at that position in the light chain of one or more of the other therapeutic antibodies, for example, as identified by the alignment in FIG. 20B.

In certain embodiments, the anti-IL31RA antigen-binding fragment transgene encodes a hyperglycosylated nemolizumab Fab, comprising a heavy chain and a light chain of SEQ ID NOs: 370 and 371, respectively, with one or more of the following mutations: L116N (heavy chain) and/or Q160N and/or Q160S and/or E195N (light chain) (see FIGS. 20A (heavy chain) and 20B (light chain)).

In certain embodiments, the anti-IL31RA antigen-binding fragment transgene encodes an antigen-binding fragment and comprises the nucleotide sequences encoding the six nemolizumab CDRs which are underlined in the heavy and light chain variable domain sequences of FIG. 29D which are spaced between framework regions, generally human framework regions, and associated with constant domains depending upon the form of the antigen-binding molecule, as is known in the art to form the heavy and/or light chain variable domain of an anti-IL31RA antibody or antigen-binding fragment thereof.

In certain embodiments, the anti-IgE antigen-binding fragment transgene comprises the nucleotide sequences encoding the heavy and light chains of the Fab portion of omalizumab (having amino acid sequences of SEQ ID NOs. 372 and 33, respectively, see Table 5 and FIG. 29E). The nucleotide sequences may be codon optimized for expression in human cells. Nucleotide sequences may, for example, comprise the nucleotide sequences of SEQ ID NO: 388 (encoding the omalizumab heavy chain Fab portion) and SEQ ID NO: 389 (encoding the omalizumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-IgE-antigen binding domain has a heavy chain Fab domain of SEQ ID NO: 372 with additional hinge region sequence starting after the C-terminal valine (V), contains all or a portion of the amino acid sequence EPKSCDKTHTCPPCPAPELLGG (SEQ ID NO: 194), and specifically, EPKSCDKTHL (SEQ ID NO: 196), EPKSCDKTHT (SEQ ID NO: 197), EPKSCDKTHTCPPCPA (SEQ ID NO: 198), EPKSCDKTHLCPPCPA (SEQ ID NO: 199), EPKSCDKTHTCPPCPAPELLGGPSVFL (SEQ ID NO: 200) or EPKSCDKTHLCPPCPAPELLGGPSVFL (SEQ ID NO: 201) as set forth in FIG. 29E. These hinge regions may be encoded by nucleotide sequences at the 3′ end of SEQ ID NO: 388 by the hinge region encoding sequences set forth in Table 6 (SEQ ID NO: 388). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 398 (Table 7) or an IgG1 Fc domain, such as SEQ ID NO: 283 or as depicted in FIG. 23, or a mutant or variant thereof. The Fc domain may be engineered for altered binding to one or more Fc receptors and/or effector function as disclosed in Section 5.1.9, infra.

In certain embodiments, the anti-IgE antigen-binding fragment transgene encodes an IgE antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 373. In certain embodiments, the anti-IgE antigen-binding fragment transgene encodes an IgE antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 372. In certain embodiments, the anti-IgE antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 373 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 372. In specific embodiments, the IgE antigen binding fragment comprises a heavy chain comprising an amino acid sequence of SEQ ID NO: 372 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29E) or are substitutions with an amino acid present at that position in the heavy chain of one or more of the other therapeutic antibodies, for example, as identified by the alignment in FIG. 20A. In specific embodiments, the IgE antigen binding fragment comprises a light chain comprising an amino acid sequence of SEQ ID NO: 373 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29E) or are substitutions with an amino acid present at that position in the light chain of one or more of the other therapeutic antibodies, for example, as identified by the alignment in FIG. 20B.

In certain embodiments, the anti-IgE antigen-binding fragment transgene encodes a hyperglycosylated omalizumab Fab, comprising a heavy chain and a light chain of SEQ ID NOs: 372 and 373, respectively, with one or more of the following mutations: L116N (heavy chain) and/or Q164N and/or Q164S and/or E199N (light chain) (see FIGS. 20A (heavy chain) and 20B (light chain)).

In certain embodiments, the anti-IgE antigen-binding fragment transgene encodes an antigen-binding fragment and comprises the nucleotide sequences encoding the six omalizumab CDRs which are underlined in the heavy and light chain variable domain sequences of FIG. 29E which are spaced between framework regions, generally human framework regions, and associated with constant domains depending upon the form of the antigen-binding molecule, as is known in the art to form the heavy and/or light chain variable domain of an anti-IgE antibody or antigen-binding fragment thereof.

In certain embodiments, the anti-TSLP antigen-binding fragment transgene comprises the nucleotide sequences encoding the heavy and light chains of the Fab portion of tezepelumab (having amino acid sequences of SEQ ID NOs. 374 and 375, respectively, see Table 5 and FIG. 29F). The nucleotide sequences may be codon optimized for expression in human cells. Nucleotide sequences may, for example, comprise the nucleotide sequences of SEQ ID NO: 390 (encoding the tezepelumab heavy chain Fab portion) and SEQ ID NO: 391 (encoding the tezepelumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-TSLP-antigen binding domain has a heavy chain Fab domain of SEQ ID NO: 374 with additional hinge region sequence starting after the C-terminal valine (V), contains all or a portion of the amino acid sequence ERKCCVECPPCPAPPVAG (SEQ ID NO: 220) or ERKCCVECPPCPA (SEQ ID NO: 221) as set forth in FIG. 29E. These hinge regions may be encoded by nucleotide sequences at the 3′ end of SEQ ID NO: 390 by the hinge region encoding sequences set forth in Table 6 (SEQ ID NO: 390). In another embodiment, the transgenes comprise the amino acid sequences encoding the full length (or substantially full length) heavy and light chains of the antibody, comprising the Fc domain at the C terminus of the heavy chain, e.g. having an amino acid sequence of an IgG2 Fc domain, such as SEQ ID NO: 284 or as depicted in FIG. 23, or a mutant or variant thereof. The Fc domain may be engineered for altered binding to one or more Fc receptors and/or effector function as disclosed in Section 5.1.9, infra.

In certain embodiments, the anti-TSLP antigen-binding fragment transgene encodes an TSLP antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 375. In certain embodiments, the anti-TSLP antigen-binding fragment transgene encodes an TSLP antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 374. In certain embodiments, the anti-TSLP antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 375 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 374. In specific embodiments, the TSLP antigen binding fragment comprises a heavy chain comprising an amino acid sequence of SEQ ID NO: 374 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29F) or are substitutions with an amino acid present at that position in the heavy chain of one or more of the other therapeutic antibodies, for example, as identified by the alignment in FIG. 20A. In specific embodiments, the TSLP antigen binding fragment comprises a light chain comprising an amino acid sequence of SEQ ID NO: 375 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 29F) or are substitutions with an amino acid present at that position in the light chain of one or more of the other therapeutic antibodies, for example, as identified by the alignment in FIG. 20B.

In certain embodiments, the anti-TSLP antigen-binding fragment transgene encodes a hyperglycosylated tezepelumab Fab, comprising a heavy chain and a light chain of SEQ ID NOs: 374 and 375, respectively, with one or more of the following mutations: M117N (heavy chain) and/or and/or Q196N (light chain) (see FIGS. 20A (heavy chain) and 20B (light chain)).

In certain embodiments, the anti-TSLP antigen-binding fragment transgene encodes an antigen-binding fragment and comprises the nucleotide sequences encoding the six tezepelumab CDRs which are underlined in the heavy and light chain variable domain sequences of FIG. 29F which are spaced between framework regions, generally human framework regions, and associated with constant domains depending upon the form of the antigen-binding molecule, as is known in the art to form the heavy and/or light chain variable domain of an anti-TSLP antibody or antigen-binding fragment thereof.

Gene Therapy Methods

Provided are methods of treating human subjects for angioedema by administration of a viral vector containing a transgene encoding an anti-IL/ILR, anti-IgE, or anti-TSLP antibody, or antigen binding fragment thereof. The antibody may be benralizumab, reslizumab, tralokinumab, nemolizumab, omalizumab, or tezepelumab and is, e.g., a full length or substantially full length antibody or Fab fragment thereof, or other antigen-binding fragment thereof. In embodiments, the patient has been diagnosed with and/or has symptoms associated with AI-Ds. Recombinant vectors used for delivering the transgene are described in Section 5.4.2. Such vectors should have a tropism for human liver or muscle cells and can include non-replicating rAAV, particularly those bearing an AAV8 or AAV9 capsid. The recombinant vectors, such as shown in FIGS. 29A-29F, can be administered in any manner such that the recombinant vector enters the liver or muscle tissue, e.g., by introducing the recombinant vector into the bloodstream. See Section 5.5.2 for details regarding the methods of treatment.

Subjects to whom such gene therapy is administered can be those responsive to anti-IL/ILR, anti-IgE, or anti-TSLP therapy. In certain embodiments, the methods encompass treating patients who have been diagnosed with AI-Ds, or have one or more symptoms associated therewith, and identified as responsive to treatment with an anti-IL/ILR, anti-IgE, or anti-TSLP antibody or considered a good candidate for therapy with an anti-IL/ILR, anti-IgE, or anti-TSLP antibody In specific embodiments, the patients have previously been treated with dupilumab, ixekizumab, secukinumab, ustekinumab, mepolizumab, benralizumab, reslizumab, tralokinumab, nemolizumab, omalizumab, or tezepelumab and have been found to be responsive to dupilumab, ixekizumab, secukinumab, ustekinumab, mepolizumab, benralizumab, reslizumab, tralokinumab, nemolizumab, omalizumab, or tezepelumab. To determine responsiveness, the anti-IL/ILR, anti-IgE, or anti-TSLP antibody or antigen-binding fragment transgene product (e.g., produced in cell culture, bioreactors, etc.) may be administered directly to the subject.

Human Post Translationally Modified Antibodies

The production of the anti-IL/ILR, anti-IgE, or anti-TSLP HuPTM mAb or HuPTM Fab, should result in a “biobetter” molecule for the treatment of angioedema accomplished via gene therapy—e.g., by administering a viral vector or other DNA expression construct encoding the anti-IL/ILR, anti-IgE, or anti-TSLP HuPTM Fab, intravenously to human subjects (patients) diagnosed with or having one or more symptoms of angioedema, to create a permanent depot in the liver or muscle tissue that continuously supplies the fully-human post-translationally modified, e.g., human-glycosylated, sulfated transgene product produced by transduced liver or muscle cells.

In specific embodiments, the anti-IL5 HuPTM mAb or antigen-binding fragment thereof has heavy and light chains with the amino acid sequences of the heavy and light chain Fab portions of benralizumab as set forth in FIG. 29A (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions Q113 and/or N163 of the heavy chain (SEQ ID NO: 364) or Q100, N158, and/or N210 of the light chain (SEQ ID NO: 365). Alternatively or in addition to, the HuPTM mAb or antigen binding-fragment thereof with the heavy and light chain variable domain sequences of benralizumab has a sulfation group at Y94 of the heavy chain (SEQ ID NO: 364) and/or Y86 and/or Y87 of the light chain (SEQ ID NO: 365). In other embodiments, the anti-IL5 HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-IL5R HuPTM mAb or antigen-binding fragment thereof has heavy and light chains with the amino acid sequences of the heavy and light chain Fab portions of reslizumab as set forth in FIG. 29B (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N35, Q108, N158, and/or N200 of the heavy chain (SEQ ID NO: 366) or Q100, N158, and/or N210 of the light chain (SEQ ID NO: 367). Alternatively or in addition to, the HuPTM mAb or antigen binding-fragment thereof with the heavy and light chain variable domain sequences of reslizumab has a sulfation group at Y93 and/or Y94 of the heavy chain (SEQ ID NO: 366) and/or Y86 and/or Y87 of the light chain (SEQ ID NO: 367). In other embodiments, the anti-IL5R HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-IL13 HuPTM mAb or antigen-binding fragment thereof has heavy and light chains with the amino acid sequences of the heavy and light chain Fab portions of tralokinumab as set forth in FIG. 29C (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N54, N164, and/or N206 of the heavy chain (SEQ ID NO: 368) or N68 and/or N172 of the light chain (SEQ ID NO: 369). Alternatively or in addition to, the HuPTM mAb or antigen binding-fragment thereof with the heavy and light chain variable domain sequences of tralokinumab has a sulfation group at Y94 and/or Y95 of the heavy chain (SEQ ID NO: 368) and/or Y86 the light chain (SEQ ID NO: 369). In other embodiments, the anti-IL13 HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-IL31RA HuPTM mAb or antigen-binding fragment thereof has heavy and light chains with the amino acid sequences of the heavy and light chain Fab portions of nemolizumab as set forth in FIG. 29D (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions Q113, N163, N196, and/or N205 of the heavy chain (SEQ ID NO: 370) or N158 and/or N210 of the light chain (SEQ ID NO: 371). Alternatively or in addition to, the HuPTM mAb or antigen binding-fragment thereof with the heavy and light chain variable domain sequences of nemolizumab has a sulfation group at Y94 and/or Y95 of the heavy chain (SEQ ID NO: 370) and/or Y86 and/or Y87 the light chain (SEQ ID NO: 371). In other embodiments, the anti-IL31RA HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-IL31RA HuPTM mAb or antigen-binding fragment thereof has heavy and light chains with the amino acid sequences of the heavy and light chain Fab portions of nemolizumab as set forth in FIG. 29D (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions Q113, N163, N196, and/or N205 of the heavy chain (SEQ ID NO: 370) or N158 and/or N210 of the light chain (SEQ ID NO: 371). Alternatively or in addition to, the HuPTM mAb or antigen binding-fragment thereof with the heavy and light chain variable domain sequences of nemolizumab has a sulfation group at Y94 and/or Y95 of the heavy chain (SEQ ID NO: 370) and/or Y86 and/or Y87 the light chain (SEQ ID NO: 371). In other embodiments, the anti-IL31RA HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-IgE HuPTM mAb or antigen-binding fragment thereof has heavy and light chains with the amino acid sequences of the heavy and light chain Fab portions of omalizumab as set forth in FIG. 29E (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N36, N59, N77, Q113, and/or N159 of the heavy chain (SEQ ID NO: 372) or Q104, N162, and/or N214 of the light chain (SEQ ID NO: 373). Alternatively or in addition to, the HuPTM mAb or antigen binding-fragment thereof with the heavy and light chain variable domain sequences of omalizumab has a sulfation group at Y94 and/or Y95 of the heavy chain (SEQ ID NO: 372) and/or Y90 and/or Y91 the light chain (SEQ ID NO: 373). In other embodiments, the anti-IgE HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In specific embodiments, the anti-TSLP HuPTM mAb or antigen-binding fragment thereof has heavy and light chains with the amino acid sequences of the heavy and light chain Fab portions of tezepelumab as set forth in FIG. 29F (with glutamine (Q) glycosylation sites; asparaginal (N) glycosylation sites, non-consensus asparaginal (N) glycosylation sites; and tyrosine-O-sulfation sites (Y) are as indicated in the legend has a glycosylation, particularly a 2,6-sialylation, at one or more of the amino acid positions N77, N80, Q114, N164, N197, and/or N206 of the heavy chain (SEQ ID NO: 374) or N68 and/or N172 of the light chain (SEQ ID NO: 375). Alternatively or in addition to, the HuPTM mAb or antigen binding-fragment thereof with the heavy and light chain variable domain sequences of tezepelumab has a sulfation group at Y94 and/or Y95 of the heavy chain (SEQ ID NO: 374) and/or Y85 and/or Y86 the light chain (SEQ ID NO: 375). In other embodiments, the anti-TSLP HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab (or a hyperglycosylated derivative of either) is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of AI-Ds, reduce the levels of pain or discomfort for the patient.

Efficacy may be monitored by scoring the symptoms or degree of inflammation in the affected tissue or area of the body, e.g., such as the skin. With regard to atopic dermatitis, efficacy can be monitored by assessing changes in the affected skin or in the quality of the patient's life over the course of treatment. One or more standardized assessments can be used to assess the change. (see e.g., Physician Global Assessment (PGA), lattice system, NPF Psoriasis Score (NPF-PS), Medical Outcome Survey Short Form 36 (SF-36), the Euro QoL, Dermatology Life Quality Index (DLQI), and the Skindex; Schram et al. (2012) Allergy; 67: 99-106: “EASI, (objective) SCORAD and POEM for atopic eczema: responsiveness and minimal clinically important difference” describing standardized assessments including Eczema Area and Severity Index (EASI) and the Severity Scoring of Atopic Dermatitis Index (SCORAD)). With regard to COPD and asthma, efficacy may be monitored by assessing changes in symptoms or by measuring airway function by peak expiratory flow (PEV) or spirometry (e.g. FEV1). With regard to eosinophilic asthma, efficacy can be monitored by assessing changes in asthma exacerbations, and in lung function (e.g. airflow obstruction, forced vital capacity, and residual volume), and asthma control. With regard to chronic idiopathic urticaria, efficacy can be monitored by assessing changes in the affected skin or changes in the degree of swelling of the lips, eyelids, or throat.

Combinations of delivery of the anti-IL/ILR, anti-IgE, or anti-TSLP HuPTM mAb or antigen-binding fragment thereof, to the liver or muscle accompanied by delivery of other available treatments are encompassed by the methods provided herein. The additional treatments may be administered before, concurrently, or subsequent to the gene therapy treatment. Available treatments for subject AI-Ds that could be combined with the gene therapy provided herein include but are not limited to antihistamines, H2 blockers, topical corticosteroids, or antidepressants for chronic idiopathic urticaria, aminosalicylates, immunomodulatory agents (e.g., azathioprine (AZA), 6-mercaptopurine (6-MP), methotrexate (MTX)), oral or topical corticosteroids (e.g., prednisone or budesonide), topical calcineurin inhibitors, inhaled corticosteroids for asthma or COPD, leukotriene modifiers, high-dose inhaled corticosteroids, and oral corticosteroids, and/or topical steroids for atopic dermatitis and administration with anti-IL/ILR, anti-IgE, or anti-TSLP agents, including but not limited to dupilumab, ixekizumab, secukinumab, ustekinumab, mepolizumab, benralizumab, reslizumab, tralokinumab, nemolizumab, omalizumab, or tezepelumab.

### 5.3.20 HuPTM Constructs and Formulations for Non-Infectious Uveitis

Compositions and methods are described for the delivery of HuPTM mAbs and antigen-binding fragments thereof, such as HuPTM Fabs, that bind to interleukin-6 receptor (IL6R), interleukin-6 (IL6), TNFα, or C5, derived from an anti-IL6R, anti-IL6, anti-TNFα, or anti-C5 antibody, such as satralizumab, sarilumab, tocilizumab, siltuximab, clazakizumab, sirukumab, olokizumab, gerilimzumab, tesidolumab, ravulizumab, adalimumab, infliximab, or golimumab (FIGS. 10B and 10D, 12A-12C, and 16A-I), indicated for treating uveitis, preferably non-infectious posterior uveitis (NIU).

In certain embodiments, provided are methods of treating panuveitis or intermediate uveitis, including associated with NIU. Accordingly, methods of treatment of NIU and panuveitis and intermediate uveitis are also provided. Uveitis is inflammation in the eye and may be associated with systemic autoimmune disorders, including Behcet's disease, sarcoidosis, juvenile chronic arthritis, Vogt-Koyanagi-Harada syndrome, multiple sclerosis, and other autoimmune indications, particularly T cell-mediated autoimmune indications. In other embodiments, the NIU is associated with ocular autoimmune disease, such as, birdshot retinochoroidopathy, multifocal choroiditis, and other white dot syndromes. Methods of treatment include methods of inhibiting, and reducing the progression of vision loss and ocular damage, such as macular scarring or atrophy, lamellar macular hole formation and optic atrophy. In particular, the method of treatment may improve visual acuity (for example, defined by the best corrected visual acuity (BCVA) score or slow the loss of visual acuity and or ocular damage. Other manifestations of NIU include vitreous haze, macular retinitis and vasculitis. Accordingly, provided are methods of treating, reducing the severity of and reducing the progression of vitreous haze, macular retinitis and/or vasculitis associated with NIU by administering the HuPTM.

In certain embodiments, the HuPTM mAb has the amino acid sequence of satralizumab, sarilumab, tocilizumab, siltuximab, clazakizumab, sirukumab, olokizumab, gerilimzumab, tesidolumab, ravulizumab, adalimumab, infliximab, or golimumab, or an antigen binding fragment thereof. Amino acid sequences of Fab fragments of the antibody are provided in FIG. 10B, 10D, 12A-12C, or 16A-I. Delivery may be accomplished via gene therapy—e.g., by administering a viral vector or other DNA expression construct encoding an IL6R-binding, IL6-binding, TNFα-binding, or C5-binding HuPTM mAb (or an antigen binding fragment and/or a hyperglycosylated derivative or other derivative, thereof) to patients (human subjects) diagnosed with one or more symptoms of non-infectious uveitis to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to IL6R, IL6, TNFα, or C5 that can be administered to deliver the HuPTM mAb or antigen binding fragment in a patient. The transgene is a nucleic acid comprising the nucleotide sequences encoding an antigen binding fragment of an antibody that binds to IL6R, IL6, TNFα, or C5, such as satralizumab, sarilumab, tocilizumab, siltuximab, clazakizumab, sirukumab, olokizumab, gerilimzumab, tesidolumab, ravulizumab, adalimumab, infliximab, golimumab, or variants thereof as detailed herein. The transgene may also encode an anti-IL6R, anti-TNFα, or anti-C5 antigen binding fragment that contains additional glycosylation sites (e.g., see Courtois et al.) Transgenes are described in detail in Sections 5.3.9, 5.3.11, or 5.3.15 above.

In preferred embodiments, the antibody that binds to TNFα is adalimumab, or variants thereof as described herein. For example, a full length or Fab adalimumab antibody may be delivered by administering an adeno-associated virus (AAV) vector encoding an adalimumab mAb (or an antigen binding fragment and/or a hyperglycosylated derivative or other derivative, thereof), such as CAG.adalimumab.IgG (SEQ ID NO: 451) or CAG.adalimumab.Fab (SEQ ID NO: 453).

Sequences encoding the anti-C5, anti-IL6R, anti-IL6, or and TNF-α antigen binding fragment transgenes can be found in Table 5 (amino acid) or Table 6 (nucleotide) or FIG. 10A, 10D, 12A-12C, or 16A-16I. The heavy and light chain sequences both have a signal or leader sequence at the N-terminus appropriate for expression and secretion in human cells, in particular, one or more cells forming the retina. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 2 that correspond to the proteins secreted by one or more cells forming the retina. Alternatively, the signal sequence may be appropriate for expression in muscle or liver cells, such as those listed in Tables 3 and 4 infra.

The anti-C5, anti-IL6, anti-IL6R, or anti-TNFα antigen binding domain can have heavy chain Fab domain of SEQ ID NOS: 39, 362, 45, 47, 49, 331, 333, 335, 337, 339, 341, 59, or 61 with additional hinge region sequence starting after the C-terminal valine (V), contains all or a portion of the amino acid sequences as set forth in FIG. 10B, 10D, 12A-C, or 16A-16I and as described in detail in Sections 5.3.9, 5.3.11, or 5.3.15.

In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NOS: 301, 303, 304, 305, 309, 310, 355-359, or 394-398 (Table 7) or an IgG1, IgG2, or IgG4 Fc domain, such as SEQ ID No. 283, 284, or 285 or as depicted in FIG. 23, or a mutant or variant thereof. The Fc domain may be engineered for altered binding to one or more Fc receptors and/or effector function as disclosed in Section 5.1.9, infra.

The anti-C5, anti-IL6, anti-IL6R, or anti-TNFα antigen binding domain can have light chain Fab domain of SEQ ID NOS: 40, 363, 46, 48, 50, 332, 334, 336, 338, 340, 342, 60, or 62 with additional hinge region sequence starting after the C-terminal valine (V), contains all or a portion of the amino acid sequences as set forth in FIG. 10B, 10D, 12A-C, or 16A-16I and as described in detail in Sections 5.3.9, 5.3.11, or 5.3.15.

In certain embodiments, the anti-C5, anti-IL6, anti-IL6R, or anti-TNFα antigen-binding fragment transgene encodes an C5, IL6, IL6R, or TNFα antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequences set forth in SEQ ID NOS: 40, 363, 46, 48, 50, 332, 334, 336, 338, 340, 342, 60, or 62. In certain embodiments, the anti-C5, anti-IL6, anti-IL6R, or anti-TNFα antigen-binding fragment transgene encodes an C5, IL6, IL6R, or TNFα antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequences set forth in SEQ ID NOS: 39, 362, 45, 47, 49, 331, 333, 335, 337, 339, 341, 59, or 61. In certain embodiments, the anti-C5, anti-IL6, anti-IL6R, or anti-TNFα antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequences set forth in SEQ ID NOS: 40, 363, 46, 48, 50, 332, 334, 336, 338, 340, 342, 60, or 62 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 39, 362, 45, 47, 49, 331, 333, 335, 337, 339, 341, 59, or 61. In specific embodiments, the anti-C5, anti-IL6, anti-IL6R, or anti-TNFα antigen binding fragment comprises a heavy chain comprising an amino acid sequence of SEQ ID NOS: 39, 362, 45, 47, 49, 331, 333, 335, 337, 339, 341, 59, or 61 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 10B, 10D, 12A-C, or 16A-I) or are substitutions with an amino acid present at that position in the heavy chain of one or more of the other therapeutic antibodies, for example, as identified by the alignment in FIG. 20A. In specific embodiments, the C5, IL6, IL6R, or TNFα antigen binding fragment comprises a light chain comprising an amino acid sequence of SEQ ID NOS: 40, 363, 46, 48, 50, 332, 334, 336, 338, 340, 342, 60, or 62 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 10B, 10D, 12A-C, or 16A-I) or are substitutions with an amino acid present at that position in the light chain of one or more of the other therapeutic antibodies, for example, as identified by the alignment in FIG. 20B.

In certain embodiments, the anti-C5, anti-IL6, anti-IL6R, or anti-TNFα antigen-binding fragment transgene encodes a hyperglycosylated Fab, comprising a heavy chain and a light chain of SEQ ID NOs: 39 and 40, 362 and 363, 45 and 46, 47 and 48, 49 and 50, 331 and 332, 333 and 334, 335 and 336, 337 and 338, 339 and 340, 341 and 342, 59 and 60, or 61 and 62, respectively, with one or more of the following mutations as indicated in FIGS. 20A (heavy chain) and 20B (light chain).

In certain embodiments, the anti-C5, anti-IL6, anti-IL6R, or anti-TNFα antigen-binding fragment transgene encodes an antigen-binding fragment and comprises the nucleotide sequences encoding the six CDRs which are underlined in the heavy and light chain variable domain sequences of FIG. 10B, 10D, 12A-12C, or 16A-16I which are spaced between framework regions, generally human framework regions, and associated with constant domains depending upon the form of the antigen-binding molecule, as is known in the art to form the heavy and/or light chain variable domain of an anti-C5, anti-IL6, anti-IL6R, or anti-TNFα antibody or antigen-binding fragment thereof.

Gene Therapy Methods

Also provided are methods of treating human subjects for non-infectious uveitis by administration of a viral vector containing a transgene encoding an anti-IL6R, anti-IL6, anti-TNFα, or anti-C5 antibody, or antigen binding fragment thereof. The antibody may be satralizumab, sarilumab, tocilizumab, siltuximab, clazakizumab, sirukumab, olokizumab, gerilimzumab, tesidolumab, ravulizumab, adalimumab, infliximab, or golimumab, and is, e.g., a Fab fragment thereof, or other antigen-binding fragment thereof.

In embodiments, the patient has been diagnosed with and/or has symptom(s) associated with non-infectious uveitis. Recombinant vectors used for delivering the transgene are described in Section 5.4.3. Such vectors should have a tropism for human retina-type cells and can include non-replicating rAAV, particularly those bearing an AAV8 capsid. Alternatively, vectors bearing an AAV2.7m8 or AAV9 capsid can be used for ocular indications. The recombinant vectors, such as the ones shown in FIG. 10B, 10D, 12A-C, or 16A-I, can be administered in any manner such that the recombinant vector enters the retina, e.g., by introducing the recombinant vector into the eye. See Section 5.5.3 for details regarding the methods of treatment.

Subjects to whom such gene therapy is administered can be those responsive to anti-IL6R, anti-IL6, anti-TNFα, or anti-C5 therapy. In certain embodiments, the methods encompass treating patients who have been diagnosed with non-infectious uveitis, or have one or more symptoms associated therewith, and identified as responsive to treatment with anti-IL6R, anti-IL6, anti-TNFα, or anti-C5 antibody or considered a good candidate for therapy with an anti-IL6R, anti-IL6, anti-TNFα, or anti-C5 antibody. In specific embodiments, the patients have previously been treated with satralizumab, sarilumab, tocilizumab, siltuximab, clazakizumab, sirukumab, olokizumab, gerilimzumab, tesidolumab, ravulizumab, adalimumab, infliximab, or golimumab, or inebilizumab, and have been found to be responsive to satralizumab, sarilumab, tocilizumab, siltuximab, clazakizumab, sirukumab, olokizumab, gerilimzumab, tesidolumab, ravulizumab, adalimumab, infliximab, or golimumab. In other embodiments, the patients have been previously treated with an anti-IL6R, anti-IL6, anti-TNFα, or anti-C5 antibody. To determine responsiveness, the anti-IL6R, anti-IL6, anti-TNFα, or anti-C5 antibody or antigen-binding fragment transgene product (e.g., produced in cell culture, bioreactors, etc.) may be administered directly to the subject.

Human Post Translationally Modified Antibodies

The production of the anti-IL6R, anti-IL6, anti-TNFα, or anti-C5 HuPTM mAb or HuPTM Fab, should result in a “biobetter” molecule for the treatment of non-infectious uveitis accomplished via gene therapy—e.g., by administering a viral vector or other DNA expression construct encoding the anti-IL6R, anti-IL6, anti-TNFα, or anti-C5 HuPTM Fab, subretinally, intravitreally, or suprachoroidally to human subjects (patients) diagnosed with or having one or more symptoms of non-infectious uveitis, to create a permanent depot in the retina that continuously supplies the fully-human post-translationally modified, e.g., human-glycosylated, sulfated transgene product produced by transduced cells of the retina.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of or relieve one or more symptoms of non-infectious uveitis, such as to reduce the levels of pain, redness of the eye, sensitivity to light, and/or other discomfort for the patient. Efficacy may be monitored by measuring a reduction in pain, redness of the eye, and/or photophobia and/or an improvement in vision. Efficacy may also be assessed by monitoring best corrected visual acuity (BCVA) and/or performing applanation tonometry, slit-lamp examination of the anterior and posterior segments, dilated indirect ophthalmoscopy, and optical coherence tomography (OTC), and comparing to baseline values.

In specific embodiments, the anti-C5, anti-IL6, anti-IL6R, or anti-TNFα HuPTM mAb or antigen-binding fragment thereof has heavy and light chains with the amino acid sequences of the heavy and light chain Fab portions as set forth in FIG. 10B, 10D, 12A-12C, or 16A-16I (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions. Alternatively or in addition to, the HuPTM mAb or antigen binding-fragment thereof with the heavy and light chain variable domain sequences has a sulfation group of the heavy chain and/or of the light chain. In other embodiments, the anti-C5, anti-IL6, anti-IL6R, or anti-TNFα HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region. See Sections 5.3.9, 5.3.11, or 5.3.15 for a detailed description of Y-sulfation sites and other post-translational modifications.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of or relieve one or more symptoms of non-infectious uveitis. Efficacy may be monitored by monitoring vision acuity, redness of the eye, sensitivity to light, and/or eye pain. For example, efficacy can be monitored by assessing change in vision acuity, redness of the eye, sensitivity to light, and/or eye pain from baseline.

Combinations of delivery of the anti-IL6, anti-IL6R, anti-TNFα, or anti-C5 HuPTM mAb or antigen-binding fragment thereof, to the retina accompanied by delivery of other available treatments are encompassed by the methods provided herein. The additional treatments may be administered before, concurrently, or subsequent to the gene therapy treatment. Available treatments for a subject with non-infectious uveitis that could be combined with the gene therapy provided herein include but are not limited to, azathioprine, methotrexate, mycophenolate mofetil, cyclosporine, cyclophosphamide, corticosteroids (local and/or systemic (oral and/or inhaled)), and others and administration with anti-TNFα, anti-IL6, anti-IL6R, or anti-C5 agents, including but not limited to adalimumab, infliximab, or golimumab.

Constructs for Delivery to Retinal Cell Types

Sections 5.3.9, 5.3.11, and 5.3.15 describe recombinant vectors that contain a transgene encoding a HuPTM mAb or HuPTM Fab (or other antigen binding fragment of the HuPTM mAb) that binds to C5, TNFα, IL6R, and IL6. Such recombinant vectors used for delivering the transgene can have a tropism for one or more human retina cell types. Such vectors can include non-replicating recombinant adeno-associated virus vectors (“rAAV”), e.g., those bearing an AAV8 capsid. Alternatively, an AAV vector bearing an AAV2.7m8 capsid can be used. However, other viral vectors may be used, including but not limited to lentiviral vectors, vaccinia viral vectors, or non-viral expression vectors referred to as “naked DNA” constructs.

In preferred embodiments, provided are constructs for gene therapy administration to a human subject, comprising an AAV vector, which comprises a viral capsid that is at least 95% identical to the amino acid sequence of an AAV8 capsid (SEQ ID NO: 143) or an AAV2.7m8 capsid (SEQ ID NO: 142); and a viral or artificial genome comprising an expression cassette flanked by AAV inverted terminal repeats (ITRs) wherein the expression cassette comprises a transgene encoding the heavy and light chains of the therapeutic antibody, operably linked to one or more regulatory sequences that control expression of the transgene in human cells (e.g., retina cell or liver cell types) that express and deliver the therapeutic antibody in a therapeutically appropriate manner as disclosed herein. In certain embodiments, the encoded AAV8 or AAV2.7m8 capsid has the sequence of SEQ ID NO: 143 or 142 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions, particularly substitutions with amino acid residues found in the corresponding position in other AAV capsids, for example, in the SUBS row of FIG. 21 which provides a comparison of the amino acid sequences of the capsid sequences of various AAVs, highlighting amino acids appropriate for substitution at different positions within the capsid sequence.

In some embodiments, the HuPTM mAb or antigen binding fragment thereof, including the HuPTM Fab transgene should be controlled by appropriate expression control elements for expression of the HuPTM Fab or HuPTM Mab in human retina or liver cell types, for example, CB7 promoter (a chicken β-actin promoter and CMV enhancer/CAG, SEQ ID NO: 411), or tissue-specific promoters, and can include other expression control elements that enhance expression of the transgene driven by the vector. Promoter sequences and sequences of other regulatory elements, such as introns, are provided in Table 1.

Gene therapy constructs for antibodies or Fabs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. The leader sequence for each of the heavy and light chains is, e.g., MYRMQLLLLIALSLALVTNS (SEQ ID NO: 146). Section 5.1.5, supra, provides specific IRES, 2A, and other linker sequences that can be used with the methods and compositions provided herein. In specific embodiments, the linker is a Furin-2A linker, for example a Furin-F2A linker RKRR(GSG)APVKQTLNFDLLKLAGDVESNPGP (SEQ ID NO: 231) or a Furin-T2A linker RKRR(GSG)EGRGSLLTCGDVEENPGP (SEQ ID NO: 429). In specific embodiments, the transgene is a nucleotide sequence that encodes the following: Signal sequence-heavy chain Fab portion-Furin-(F/T)2A linker-signal sequence-light chain Fab portion. See FIGS. 10B and 10D for sequences for tesidolumab and ravulizumab Fab expression, respectively; FIGS. 12A to 12C for a sequence for adalimumab, infliximab, and golimumab Fab expression, respectively; and FIGS. 16A to 16I for sequences for satralizumab, sarilumab, siltuximab, clazakizumab, sirukumab, olokizumab, gerilimzumab, and tocilizumab, inebilizumab Fab expression, respectively. In an alternative embodiment, the construct also comprises the nucleotide sequence encoding the Fc domain for the therapeutic antibody (see Table 7 or FIG. 23) for expression of the full length mAb. Nucleotide sequences for adalimumab mAb and Fab single chain construct transgenes are described in Table 17. Nucleic acid sequences coding for the component sequences of the heavy and light chains of adalimumab, either the full length (SEQ ID NOS: 444 and 448) or Fab fragments (components encoded by SEQ ID NOS: 445 and 446 (heavy chain) and 498-450 (light chain) are also provided.

In a specific embodiment, the constructs described herein comprise the following components: (1) AAV2 inverted terminal repeats that flank the expression cassette; (2) Control elements, which include a) the CB7 promoter (SEQ ID NO: 411), comprising the CMV enhancer/chicken (β-actin promoter, b) a chicken (β-actin intron and c) a rabbit β-globin poly A signal; and (3) nucleic acid sequences coding for the heavy and light chains of the C5-binding, TNFα-binding, IL6R-binding, and IL6-binding Fab, separated by a self-cleaving furin (F)/(F/T)2A linker (SEQ ID NOS: 231 or 429, respectively), ensuring expression of equal amounts of the heavy and the light chain polypeptides. An exemplary construct is provided in FIG. 1.

In a preferred embodiment, the construct described herein comprise the following components: (1) AAV2 inverted terminal repeats that flank the expression cassette; (2) Control elements, which include a) the CB7 promoter (SEQ ID NO: 411), comprising the CMV enhancer/chicken β-actin promoter, b) a chicken β-actin intron and c) a rabbit β-globin poly A signal; and (3) nucleic acid sequences coding for the component sequences of the heavy and light chains of adalimumab, either the full length (SEQ ID NOS: 444 and 448) or Fab fragments (components encoded by SEQ ID NOS: 445 and 446 (heavy chain) and 498-450 (light chain), separated by a self-cleaving furin (F)/T2A linker (SEQ ID NOS: 429), ensuring expression of equal amounts of the heavy and the light chain polypeptides.

In specific embodiments, provided are AAV vectors comprising a viral capsid that is at least 95% identical to the amino acid sequence of an AAV8 capsid (SEQ ID NO: 143) or of an AAV2.7m8 (SEQ ID NO: 142); and an artificial genome comprising an expression cassette flanked by AAV inverted terminal repeats, wherein the expression cassette comprises a transgene encoding an anti-C5 anti-TNFα, anti-IL6R, and anti-IL6 mAb, or an antigen-binding fragment thereof, operably linked to one or more regulatory sequences that control expression of the transgene in one or more retina cell types (such as human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells).

In a preferred embodiment, provided is an AAV comprising a viral capsid that is at least 95% identical to the amino acid sequence of an AAV8 capsid (SEQ ID NO: 143); and an artificial genome comprising an expression cassette flanked by AAV inverted terminal repeats (ITRs), wherein the expression cassette comprises a transgene encoding an adalimumab mAb (SEQ ID NO: 451), or an antigen-binding fragment thereof (SEQ ID NO: 453), operably linked to one or more regulatory sequences that control expression of the transgene in one or more retina cell types (such as human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells).

Administration for Delivering to Retinal Type Cells

Therapeutically effective doses of the recombinant vector described above should be administered in any manner such that the recombinant vector enters the retina, e.g., by introducing the recombinant vector directly into the eye. In specific, embodiments, the vector is administered subretinally (a surgical procedure performed by trained retinal surgeons that involves a partial vitrectomy with the subject under local anesthesia, and injection of the gene therapy into the retina, or intravitreally, or suprachoroidally such as by microinjection or microcannulation. Subretinal, intravitreal or suprachoroidal administration should result in expression of the soluble transgene product in one or more of the following retinal cell types: human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells. The expression of the encoded anti-C5, anti-TNFα, anti-IL6R, and anti-IL6 antibody results in delivery and maintenance of the transgene product in the retina. Pharmaceutical compositions suitable for administration comprise a suspension of the recombinant vector comprising the transgene encoding the anti-C5, anti-TNFα, anti-IL6R, and anti-IL6 antibody, or antigen-binding fragment thereof, in a formulation buffer comprising a physiologically compatible aqueous buffer. The formulation buffer can comprise one or more of a polysaccharide, a surfactant, polymer, or oil.

Full Length Expression of mAb in Retinal Cell Types

In a specific embodiment for expressing an intact or substantially intact mAb in retinal cell types, the constructs described herein comprise the following components: (1) AAV2 inverted terminal repeats that flank the expression cassette; (2) Control elements, which include a) the CB7 promoter, comprising the CMV enhancer/chicken (β-actin promoter, b) a chicken β-actin intron and c) a rabbit β-globin poly A signal; and (3) nucleic acid sequences coding for the heavy chain Fab of an anti-C5 (e.g., tesidolumab, ravulizumab), anti-TNFα (e.g., adalimumab, infliximab, and golimumab), anti-IL6R (e.g., satralizumab, sarilumab, and tocilizumab), anti-IL6 (e.g. siltuximab, clazakizumab, sirukumab, olokizumab, and gerilimzumab), an Fc polypeptide associated with the therapeutic antibody (Table 6 and X) or of the same IgG isotype as the native form of the therapeutic antibody, such as an IgG isotype amino acid sequence from FIG. 23, and a light chain of an anti-C5 (e.g., tesidolumab and ravulizumab), anti-TNFα (e.g., adalimumab, infliximab, and golimumab), anti-IL6R (e.g., satralizumab, sarilumab, and tocilizumab), and anti-IL6 (e.g. siltuximab, clazakizumab, sirukumab, olokizumab, and gerilimzumab), wherein the heavy chain (Fab and Fc polypeptide) and light chain are separated by a self-cleaving furin (F)/(T/F)2A or flexible linker, ensuring expression of equal amounts of the heavy and the light chain polypeptides. An exemplary construct is provided in FIG. 1.

In specific embodiments, provided are AAV vectors comprising a viral capsid that is at least 95% identical to the amino acid sequence of an AAV8 capsid (SEQ ID NO: 143) or of an AAV2.7m8 (SEQ ID NO: 142); and an artificial genome comprising an expression cassette flanked by AAV inverted terminal repeats (ITRs), wherein the expression cassette comprises a transgene encoding an intact or substantially intact anti-C5, anti-TNFα, anti-IL6R, or anti-IL6 mAb, operably linked to one or more regulatory sequences that control expression of the transgene in one or more retina cell types (such as human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells).

### 5.3.21. Anti-C5 HuPTM Constructs and Formulations for Myasthenia Gravis

Compositions and methods are described for the delivery of HuPTM mAbs and antigen-binding fragments thereof, such as HuPTM Fabs, that bind to complement component 5 (anti-C5) derived from anti-C5 antibody, such as ravulizumab (FIG. 10D), and indicated for treating myasthenia gravis. In certain embodiments, the HuPTM mAb has the amino acid sequence of romosozumab or an antigen binding fragment thereof. The amino acid sequence of Fab fragment of this antibody is provided in FIG. 10D. Delivery may be accomplished via gene therapy—e.g., by administering a viral vector or other DNA expression construct encoding an C5-binding HuPTM mAb (or an antigen binding fragment and/or a hyperglycosylated derivative or other derivative, thereof) to patients (human subjects) diagnosed with myasthenia gravis to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to C5 that can be administered to deliver the HuPTM mAb or antigen binding fragment in a patient. The transgene is a nucleic acid comprising the nucleotide sequences encoding an antigen binding fragment of an antibody that binds to C5, such as ravulizumab or variants thereof as detailed herein. The transgene may also encode an anti-C5 antigen binding fragment that contains additional glycosylation sites (e.g., see Courtois et al.).

In certain embodiments, the anti-C5 antigen-binding fragment transgene comprises the nucleotide sequences encoding the heavy and light chains of the Fab portion of ravulizumab (having amino acid sequences of SEQ ID NOs. 362 and 363, respectively, see Table 5 and FIG. 10D). The nucleotide sequences may be codon optimized for expression in human cells. Nucleotide sequences may, for example, comprise the nucleotide sequences of SEQ ID NO: 378 (encoding the ravulizumab heavy chain Fab portion) and SEQ ID NO: 379 (encoding the ravulizumab light chain Fab portion) as set forth in Table 6. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-C5-antigen binding domain has a heavy chain Fab domain of SEQ ID NO: 362 with additional hinge region sequence starting after the C-terminal valine (V), contains all or a portion of the amino acid sequence contains all or a portion of the amino acid sequence ERKCCVECPPCPAPPVAG (SEQ ID NO: 220) or ERKCCVECPPCPA (SEQ ID NO: 221) as set forth in FIG. 10D. These hinge regions may be encoded by nucleotide sequences at the 3′ end of SEQ ID NO: 378 by the hinge region encoding sequences set forth in Table 6 (SEQ ID NO: 378). In another embodiment, the transgenes comprise the amino acid sequences encoding the full length (or substantially full length) heavy and light chains of the antibody, comprising the Fc domain at the C terminus of the heavy chain, e.g having an amino acid sequence of SEQ ID NO: 393 (Table 7) or an IgG2 Fc domain, such as SEQ ID No. 284 or as depicted in FIG. 23, or a mutant or variant thereof. The Fc domain may be engineered for altered binding to one or more Fc receptors and/or effector function as disclosed in Section 5.1.9, infra.

In certain embodiments, the anti-C5 antigen-binding fragment transgene encodes an C5 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 363. In certain embodiments, the anti-C5 antigen-binding fragment transgene encodes an C5 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 362. In certain embodiments, the anti-C5 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 363 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 362. In specific embodiments, the C5 antigen binding fragment comprises a heavy chain comprising an amino acid sequence of SEQ ID NO: 362 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 10D) or are substitutions with an amino acid present at that position in the heavy chain of one or more of the other therapeutic antibodies, for example, as identified by the alignment in FIG. 20A. In specific embodiments, the C5 antigen binding fragment comprises a light chain comprising an amino acid sequence of SEQ ID NO: 363 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 10D) or are substitutions with an amino acid present at that position in the light chain of one or more of the other therapeutic antibodies, for example, as identified by the alignment in FIG. 20B.

In certain embodiments, the anti-C5 antigen-binding fragment transgene encodes a hyperglycosylated ravulizumab Fab, comprising a heavy chain and a light chain of SEQ ID NOs: 363 and 362, respectively, with one or more of the following mutations L117N (heavy chain), Q160N or Q160S (light chain), and/or E195N (light chain) (see FIG. 20A (heavy chain) and B (light chain)).

In certain embodiments, the anti-C5 antigen-binding fragment transgene encodes an antigen-binding fragment and comprises the nucleotide sequences encoding the six romosozumab CDRs which are underlined in the heavy and light chain variable domain sequences of FIG. 10D which are spaced between framework regions, generally human framework regions, and associated with constant domains depending upon the form of the antigen-binding molecule, as is known in the art to form the heavy and/or light chain variable domain of an anti-C5 antibody or antigen-binding fragment thereof.

Gene Therapy Methods

Provided are methods of treating human subjects for myasthenia gravis by administration of a viral vector containing a transgene encoding an anti-C5 antibody, or antigen binding fragment thereof. The antibody may be ravulizumab, and is e.g. a full length or substantially full length antibody or Fab fragment thereof, or other antigen-binding fragment thereof. In embodiments, the patient has been diagnosed with and/or has symptoms associated with myasthenia gravis. Recombinant vectors used for delivering the transgene are described in Section 5.4.2. Such vectors should have a tropism for human liver or muscle cells and can include non-replicating rAAV, particularly those bearing an AAV8 or AAV9 capsid. The recombinant vector, such as shown in FIG. 10D, can be administered in any manner such that the recombinant vector enters the liver or muscle tissue, e.g., by introducing the recombinant vector into the bloodstream. See Section 5.5.2 for details regarding the methods of treatment.

Subjects to whom such gene therapy is administered can be those responsive to anti-C5 therapy. In certain embodiments, the methods encompass treating patients who have been diagnosed with myasthenia gravis, or have one or more symptoms associated therewith, and identified as responsive to treatment with an anti-C5 antibody or considered a good candidate for therapy with an anti-C5 antibody. In specific embodiments, the patients have previously been treated with ravulizumab, and have been found to be responsive to ravulizumab. To determine responsiveness, the anti-C5 antibody or antigen-binding fragment transgene product (e.g., produced in cell culture, bioreactors, etc.) may be administered directly to the subject.

Human Post Translationally Modified Antibodies

The production of the anti-C5 HuPTM mAb or HuPTM Fab, should result in a “biobetter” molecule for the treatment of myasthenia gravis accomplished via gene therapy—e.g., by administering a viral vector or other DNA expression construct encoding the anti-C5 HuPTM Fab, intravenously to human subjects (patients) diagnosed with or having one or more symptoms of osteoporosis or bone loss, to create a permanent depot in the liver or muscle tissue that continuously supplies the fully-human post-translationally modified, e.g., human-glycosylated, sulfated transgene product produced by transduced liver or muscle cells.

The cDNA construct for the anti-C5 HuPTMmAb or anti-C5 HuPTM Fab should include a signal peptide that ensures proper co- and post-translational processing (glycosylation and protein sulfation) by the transduced liver or muscle cells. For example, the signal sequence may be MYRMQLLLLIALSLALVTNS (SEQ ID NO: 146). Alternatively, the signal sequence may have an amino acid sequence selected from any one of the signal sequences set forth in Table 3 or 4 that correspond to the proteins secreted by myocytes or hepatocytes, respectively.

As an alternative, or an additional treatment to gene therapy, the anti-C5 HuPTM mAb or HuPTM Fab can be produced in human cell lines by recombinant DNA technology, and administered to patients diagnosed with osteoporosis or bone loss, or for whom therapy for osteoporosis or bone loss is considered appropriate.

In specific embodiments, the anti-C5 HuPTM mAb or antigen-binding fragment thereof has heavy and light chains with the amino acid sequences of the heavy and light chain Fab portions of ravulizumab as set forth in FIG. 10D (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N63, N106, Q114, N164, N197, and/or N206 of the heavy chain (SEQ ID NO: 362) or N28, Q100, N158, and/or N210 of the light chain (SEQ ID NO: 363). Alternatively or in addition to, the HuPTM mAb or antigen binding-fragment thereof with the heavy and light chain variable domain sequences of ravulizumab has a sulfation group at Y94 and/or Y95 of the heavy chain (SEQ ID NO: 362) and/or Y86 and/or Y86 of the light chain (SEQ ID NO: 363). In other embodiments, the anti-C5 HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of myasthenia gravis. Efficacy may be monitored by evaluating changes in endurance or fatigability from baseline, e.g. by using the Quantitative Myasthenia Gravis Score (QMGS), Manual Muscle Test (MMT), and/or Myasthenia Muscle Score (see Barnett C et al. Neurol Clin. 2018 May; 36(2): 339-353). For example, the QMSC score assesses changes in ptosis, diplopia, orbicularis oculi weakness, swallowing a cup of water, speech, percent predicted forced vital capacity, grip strength (2 items), arm endurance (2 items), leg endurance (2 items), and neck flexion endurance.

Combinations of delivery of the anti-C5 HuPTM mAb or antigen-binding fragment thereof, to the liver or muscles accompanied by delivery of other available treatments are encompassed by the methods provided herein. The additional treatments may be administered before, concurrently, or subsequent to the gene therapy treatment. Available treatments for myasthenia gravis that could be combined with the gene therapy provided herein include but are not limited to pyridostigmine, corticosteroids, or immunosuppressants, and administration with anti-C5 agents, including but not limited to ravulizumab.

TABLE 5
Table of Fab Fragment Amino Acid Sequences

| | Chain/ | |
|---|---|---|
| | SEQ ID | |
| mAb | NO. | Sequence |
| Solanezumab | Heavy/ | EVQLVESGGG LVQPGGSLRL SCAASGFTFS RYSMSWVRQA |
| | SEQ ID | PGKGLELVAQ INSVGNSTYY PDTVKGRFTI SRDNAKNTLY |
| | NO: 1 | LQMNSLRAED TAVYYCASGD YWGQGTLVTV SSASTKGPSV |
| | | FPLAPSSKST SGGTAALGCL VKDYFPEPVT VSWNSGALTS |
| | | GVHTFPAVLQ SSGLYSLSSV VTVPSSSLGT QTYICNVNHK |
| | | PSNTKVDKKV EPKSCD +/- KTHT(or KTHL) +/- CPPCPA +/- |
| | | PELLGGPSVFL |
| Solanezumab | Light/ | DVVMTQSPLS LPVTLGQPAS ISCRSSQSLI YSDGNAYLHW |
| | SEQ ID | FLQKPGQSPR LLIYKVSNRF SGVPDRFSGS GSGTDFTLKI |
| | NO: 2 | SRVEAEDVGV YYCSQSTHVP WTFGQGTKVE IKRTVAAPSV |
| | | FIFPPSDEQL KSGTASVVCL LNNFYPREAK VQWKVDNALQ |
| | | SGNSQESVTE QDSKDSTYSL SSTLTLSKAD YEKHKVYACE |
| | | VTHQGLSSPV TKSFNRGEC |
| GSK933776 | Heavy/ | EVQLVESGGG LVQPGGSLRL SCA A/V SGFTFS DNGMAWVRQA |
| | SEQ ID | PGKGLEWVSF ISNLAYSIDY ADTVTGRFTI SRDNAKNSLY |
| | NO: 3 | LQMNSLRAED TAVYYCVSGT WFAYWGQGTL VTVSSASTKG |
| | | PSVFPLAPSS KSTSGGTAAL GCLVKDYFPE PVTVSWNSGA |
| | | LTSGVHTFPA VLQSSGLYSL SSVVTVPSSS LGTQTYICNV |
| | | NHKPSNTKVD KKVEPKSCD +/- KTHT (or KTHL)+/- |
| | | CPPCPA +/- PELAGAPSVFL |
| GSK933776 | Light/ | DIVMTQSPLS LPVTPGEPAS ISCRVSQSLL HSNGYTYLHW |
| | SEQ ID | YLQKPGQSPQ LLIYKVSNRF SGVPDRFSGS GSGTDFTLKI |
| | NO: 4 | SRVEAEDVGV YYCSQTRHVP YTFGGGTKVE IKRTVAAPSV |
| | | FIFPPSDEQL KSGTASVVCL LNNFYPREAK VQWKVDNALQ |
| | | SGNSQESVTE QDSKDSTYSL SSTLTLSKAD YEKHKVYACE |
| | | VTHQGLSSPV TKSFNRGEC |
| AL-001 | Heavy/ | QVQLVQSGAE VKKPGASVKV SCKASGYTFT KYYMSWVRQA |
| | SEQ ID | PGQGLEWMGI INPIGGSTSY AQKFQGRVTM TRDTSTSTVY |
| | NO: 5 | MELSSLRSED TAVYYCARDP SGIALAGPAS RGYQGMDVWG |
| | | QGTTVTVSSA STKGPSVFPL APSSKSTSGG TAALGCLVKD |
| | | YFPEPVTVSW NSGALTSGVH TFPAVLQSSG LYSLSSVVTV |
| | | PSSSLGTQTY ICNVNHKPSN TKVDKRVEPK SCD +/- KTHT |
| | | (or KTHL) +/-CPPCPA +/- PELLGGPSVFL |
| AL-001 | Light/ | EIVMTQSPAT LSVSPGERAT LSCRASQSVS SNLAWYQQKP |
| | SEQ ID | GQAPRLLIYG ASTRATGIPA RFSGSGSGTE FTLTISSLQS |
| | NO: 6 | EDFAVYYCQQ ARLGPWTFGG GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| ABBV-8E12 | Heavy/ | EVKVVESGGG LVQPGGSMKL SCVVSGFTFS NYWVNWVRQA |
| | SEQ ID | PGKGLEWVAQ IRLKSDNYAT HYEESVKGRF TISRDDSKSS |
| | NO: 7 | VYLQMNNLRA EDSGIYYCTN WEDYWGQGTT VTVSSASTKG |
| | | PSVFPLAPCS RSTSESTAAL GCLVKDYFPE PVTVSWNSGA |
| | | LTSGVHTFPA VLQSSGLYSL SSVVTVPSSS LGTKTYTCNV |
| | | DHKPSNTKVD KRVESKY +/- GPPCPPCPA (or GPPCPSCPA)+/- |
| | | PEFLGGPSVFL |
| ABBV-8E12 | Light/ | DIVLTQSPDS LAVSLGERAT ISCRASQSVS TSRYSYIHWY |
| | SEQ ID | QQKPGQFPKL LIKYASNLES GVPSRFSGSG SGTDFTLNIH |
| | NO: 8 | PLEPEDFATY YCHHSWEIPL TFGQGTKLEI KRTVAAPSVF |
| | | IFPPSDEQLK SGTASVVCLL NNFYPREAKV QWKVDNALQS |
| | | GNSQESVTEQ DSKDSTYSLS STLTLSKADY EKHKVYACEV |
| | | THQGLSSPVT KSFNRGEC |
| UCB-0107 | Heavy/ | EVQLQESGPG LVKPSETLSL TCTVSGFSLT SNDIAWIRQP |
| | SEQ ID | PGKGLEWMGT IWTDGSTNYN A/T AVQSRVTIS VDTSKNQFSL |
| | NO: 9 | KLSSVTAADT AVYYCARHRL YYGAFDYWGQ GTMVTVSSAS |
| | | TKGPSVFPLA PCSRSTSEST AALGCLVKDY FPEPVTVSWN |
| | | SGALTSGVHT FPAVLQSSGL YSLSSVVTVP SSSLGTKTYT |
| | | CNVDHKPSNT KVDKRVESKY +/- GPPCPPCPA (or |
| | | GPPCPSCPA)+/- PEFLGGPSVFL |
| UCB-0107 | Light/ | DIVMTQTPLS LSVTPGQPAS ISCRSSQSLE YSDGYTYLEW |
| | SEQ ID | YLQKPGQSPQ LLIYEVSNRF SGVPDRFSGS GSGTDFTLKI |
| | NO: 10 | SRVEAEDVGV YYCFQATHNP YTFGQGTKLE IKRTVAAPSV |
| | | FIFPPSDEQL KSGTASVVCL LNNFYPREAK VQWKVDNALQ |
| | | SGNSQESVTE QDSKDSTYSL SSTLTLSKAD YEKHKVYACE |
| | | VTHQGLSSPV TKSFNRGEC |
| NI-105 | Heavy/ | QVQLVESGGG VVQPGRSLRV SCAASGFTFS SYDMHWVRQA |
| | SEQ ID | PGKGLEWVAV IWFDGSNEFY ADSVKGRFTI SRDNSKNTLF |
| | NO: 11 | QMNSLRAEDT AVYYCARDLG ASVTTSNAEN FHHWGQGTLV |
| | | TVSSASTKGP SVFPLAPSSK STSGGTAALG CLVKDYFPEP |
| | | VTVSWNSGAL TSGVHTFPAV LQSSGLYSLS SVVTVPSSSL |
| | | GTQTYICNVN HKPSNTKVDK KVEPKSCD +/- KTHT (or KTHL)+/- |
| | | CPPCPA +/- PELAGAPSVFL |
| NI-105 | Light/ | SYELTQPPSV SVSPGQTARI TCSGDALPKR YVYWYQQKSG |
| | SEQ ID | QAPVLVIYED SKRPSGIPET FSGSSSGTMA TLTISGAQVE |
| | NO: 12 | DEADYYCYST DSNGHHWVFG GGTKLTVLGQ PKAAPSVTLF |
| | | PPSSEELQAN KATLVCLISD FYPGAVTVAW KADSSPVKAG |
| | | VETTTPSKQS NNKYAASSYL SLTPEQWKSH RSYSCQVTHE |
| | | GSTVEKTVAP TCS |
| VX15/2503 | Heavy/ | QVQLVQSGAE VKKPGSSVKV SCKASGYSFS DYYMHWVRQA |
| | SEQ ID | PGQGLEWMGQ INPTTGGASY NQKFKGKATI TVDKSTSTAY |
| | NO: 13 | MELSSLRSED TAVYYCARYY YGRHFDVWGQ GTTVTVSSAS |
| | | TKGPSVFPLA PCSRSTSEST AALGCLVKDY FPEPVTVSWN |
| | | SGALTSGVHT FPAVTVSSVL QSSGLYSLSS VVTVPSSSLG |
| | | TKTYTCNVDH KPSNTKVDKR VESKY +/- GPPCPPCPA(or |
| | | GPPCPSCPA) +/- PEFLGGPSVFL |
| VX15/2503 | Light/ | DIVMTQSPDS LAVSLGERAT INCKASQSVD YDGDSYMNWY |
| | SEQ ID | QQKPGQPPKL LIYAASNLES GVPDRFSGSG SGTDFTLTIS |
| | NO: 14 | SLQAEDVAVY YCQQSNEDPY TFGQGTKLEI KFIFPPSDEQ |
| | | LKSGTASVVC LLNNFYPREA KVQWKVDNAL QSGNSQESVT |
| | | EQDSKDSTYS LSSTLTLSKA DYEKHKVYAC EVTHQGLSSP |
| | | VTKSFNRGEC |
| Prasinezumab | Heavy/ | EVQLVESGGG LVQPGGSLRL SCAASGFTFS NYGMSWVRQA |
| | SEQ ID | PGKGLEWVAS ISSGGGSTYY PDNVKGRFTI SRDDAKNSLY |
| | NO: 15 | LQMNSLRAED TAVYYCARGG AGIDYWGQGT LVTVSSASTK |
| | | GPSVFPLAPS SKSTSGGTAA LGCLVKDYFP EPVTVSWNSG |
| | | ALTSGVHTFP AVLQSSGLYS LSSVVTVPSS SLGTQTYICN |
| | | VNHKPSNTKV DKRVEPKSCD +/- KTHT(or KTHL) +/- |
| | | CPPCPA +/- PELLGGPSVFL |
| Prasinezumab | Light/ | DIQMTQSPSS LSASVGDRVT ITCKSIQTLL YSSNQKNYLA |
| | SEQ ID | VFQQKPGKAP KLLIYWASIR KSGVPSRFSG SGSGTDFTLT |
| | NO: 16 | ISSLQPEDLA TYYCQQYYSY PLTFGGGTKL EIKRTVAAPS |
| | | VFIFPPSDEQ LKSGTASVVC LLNNFYPREA KVQWKVDNAL |
| | | QSGNSQESVT EQDSKDSTYS LSSTLTLSKA DYEKHKVYAC |
| | | EVTHQGLSSP VTKSFNRGEC |
| NI-202 | Heavy/ | EVQLVESGAE VKKPGASVKV SCKASGYTFT NYAMHWVRQA |
| | SEQ ID | PGQRLEWMGW INAGNGKRKY SQKFQDRVTI NRDTSASTIY |
| | NO: 17 | MELSSLGSED TAVYYCAREE DHAGSGSYLS MDVWGQGTLV |
| | | TVSSASTKGP SVFPLAPSSK STSGGTAALG CLVKDYFPEP |
| | | VTVSWNSGAL TSGVHTFPAV LQSSGLYSLS SVVTVPSSSL |
| | | GTQTYICNVN HKPSNTKVDK RVEPKSCD +/- KTHT(or KTHL) +/- |
| | | CPPCPA +/- PELLGGPSVFL |
| NI-202 | Light/ | DIQMTQSPSS LSASVGDRVT ITCKSIQTLL YSSNQKNYLA |
| | SEQ ID | WFQQKPGKAP KLLIYWASIR KSGVPSRFSG SGSGTDFTLT |
| | NO: 18 | ISSLQPEDLA TYYCQQYYSY PLTFGGGTKL EIKRTVAAPS |
| | | VFIFPPSDEQ LKSGTASVVC LLNNFYPREA KVQWKVDNAL |
| | | QSGNSQESVT EQDSKDSTYS LSSTLTLSKA DYEKHKVYAC |
| | | EVTHQGLSSP VTKSFNRGEC |
| MEDI- | Heavy/ | EVQLLESGGG LVQPGGSLRL SCAASGFTFS SYAMSWVRQA |
| 1341/TAK341 | SEQ ID | PGKGLEWVSS ISHLGGSTYY ADSVKGRFTI SRDNSKNTLY |
| | NO: 19 | LQMNSLRAED TAVYYCAGGA NHGKYYYGMD KWGQGTTVTV |
| | | SSASTKGPSV FPLAPSSKST SGGTAALGCL VKDYFPEPVT |
| | | VSWNSGALTS GVHTFPAVLQ SSGLYSLSSV VTVPSSSLGT |
| | | QTYICNVNHK PSNTKVDKRV EPKSCD +/- KTHT (or KTHL) +/- |
| | | CPPCPA +/- PEFEGGPSVFL |
| MEDI- | Light/ | QAVLTQPASL SASPGASASL TCTLRSGAPL PKYRIYWYQQ |
| 1341/TAK341 | SEQ ID | KPGSPPQYLL RYKSDADKHQ GSGVPSRFSG SKDASANAGI |
| | NO: 20 | LLISGLQSED EADYYCMVWD HGVWYFGGGT KLTVLGQPKA |
| | | APSVTLFPPS SEELQANKAT LVCLISDFYP GAVTVAWKAD |
| | | SSPVKAGVET TTPSKQSNNK YAASSYLSLT PEQWKSHRSY |
| | | SCQVTHEGST VEKTVAPTEC S |
| NI-204.10D12 | Heavy/ | EVQLVESGGD LVRPGGSLRL SCVASGFTFS NYWMHWVRQA |
| | SEQ ID | PGQRPVWVSR TNTDGRNTAY ADYAKGRFTI SRDNAKSTLY |
| | NO: 21 | LQLNSLRAED TAVYFCARLR RNVADQITHN YYMDVWGKGT |
| | | LVTVSSASTK GPSVFPLAPS SKSTSGGTAA LGCLVKDYFP |
| | | EPVTVSWNSG ALTSGVHTFP AVLQSSGLYS LSSVVTVPSS |
| | | SLGTQTYICN VNHKPSNTKV DKKVEPKSCD +/- KTHT |
| | | (or KTHL) +/- CPPCPA +/- PEAAGGPSVFL |
| NI-204.10D12 | Light/ | DIQMTQSPSS LSASVGDRVT ACRASQSVGT YLNWYQQKRG |
| | SEQ ID | KAPKLLIFAA SSLQSGVPSR FSGSGSGTDF TLTISSLQPE |
| | NO: 22 | DFATYYCQQS YSSPPTFGQG TKVEIKRTVA APSVFIFPPS |
| | | DEQLKSGTAS VVCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| NI-204.12G7 | Heavy/ | QVQLVQSGAE VKKPGASVTL SCKASGYTFT AYYIHWVRQA |
| | SEQ ID | REQGLEWMGV INPSTGTTFY AQNFPDRVSV TRDTSTSTVF |
| | NO: 23 | MELHNLKSED TAVYYCARAI SEHGSGSYSP YYWGQGTLVT |
| | | VSSASTKGPS VFPLAPSSKS TSGGTAALGC LVKDYFPEPV |
| | | TVSWNSGALT SGVHTFPAVL QSSGLYSLSS VVTVPSSSLG |
| | | TQTYICNVNH KPSNTKVDKK VEPKSCD +/- KTHT (or KTHL)+/- |
| | | CPPCPA +/- PEAAGGPSVFL |
| NI-204.12G7 | Light/ | SYELTQPPSV SVSLGQMAAI TCSGEALPKK YGYWYQQKPG |
| | SEQ ID | QVPVLLIYRD VERPSGVPDR FSGSSSGTMV TLTISGVQAE |
| | NO: 24 | DEADYYCLSA DSSGTWVFGG GTKLTVLGQP KANPTVTLFP |
| | | PSSEELQANK ATLVCLISDF YPGAVTVAWK ADGSPVKAGV |
| | | ETTKPSKQSN NKYAASSYLS LTPEQWKSHR SYSCQVTHEG |
| | | STVEKTVAPT ECS |
| Eptinezumab | Heavy/ | EVQLVESGGG LVQPGGSLRL SCAVSGIDLS GYYMNWVRQA |
| | SEQ ID | PGKGLEWVGV IGINGATYYA SWAKGRFTIS RDNSKTTVYL |
| | NO: 25 | QMNSLRAEDT AVYFCARGDI WGQGTLVTVS SASTKGPSVF |
| | | PLAPSSKSTS GGTAALGCLV KDYFPEPVTV SWNSGALTSG |
| | | VHTFPAVLQS SGLYSLSSVV TVPSSSLGTQ TYICNVNHKP |
| | | SNTKVDARVE PKSCD +/- KTHT (or KTHL) +/- |
| | | CPPCPA +/- PELLGGPSVTL |
| Eptinezumab | Light/ | QVLTQSPSSL SASVGDRVTI NCQASQSVYH NTYLAWYQQK |
| | SEQ ID | PGKVPKQLIY DASTLASGVP SRFSGSGSGT DFTLTISSLQ |
| | NO: 26 | PEDVATYYCL GSYDCTNGDC FVFGGGTKVE IKRTVAAPSV |
| | | FIFPPSDEQL KSGTASVVCL LNNFYPREAK VQWKVDNALQ |
| | | SGNSQESVTE QDSKDSTYSL SSTLTLSKAD YEKHKVYACE |
| | | VTHQGLSSPV TKSFNRGEC |
| Fremanezumab | Heavy/ | EVQLVESGGG LVQPGGSLRL SCAASGFTFS NYWISWVRQA |
| | SEQ ID | PGKGLEWVAE IRSESDASAT HYAEAVKGRF TISRDNAKNS |
| | NO: 27 | LYLQMNSLRA EDTAVYYCLA YFDYGLAIQN YWGQGTLVTV |
| | | SSASTKGPSV FPLAPCSRST SESTAALGCL VKDYFPEPVT |
| | | VSWNSGALTS GVHTFPAVLQ SSGLYSLSSV VTVPSSNFGT |
| | | QTYTCNVDHK PSNTKVDKTV ERKCCVE +/- CPPCPA +/- |
| | | PPVAG |
| Fremanezumab | Light/ | EIVLTQSPAT LSLSPGERAT LSCKASKRVT TYVSWYQQKP |
| | SEQ ID | GQAPRLLIYG ASNRYLGIPA RFSGSGSGTD FTLTISSLEP |
| | NO: 28 | EDFAVYYCSQ SYNYPYTFGQ GTKLEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Galcanezumab | Heavy/ | QVQLVQSGAE VKKPGSSVKV SCKASGYTFG NYWMQWVRQA |
| | SEQ ID | PGQGLEWMGA IYEGTGKTVY IQKFADRVTI TADKSTSTAY |
| | NO: 29 | MELSSLRSED TAVYYCARLS DYVSGFGYWG QGTTVTVSSA |
| | | STKGPSVFPL APCSRSTSES TAALGCLVKD YFPEPVTVSW |
| | | NSGALTSGVH TFPAVLQSSG LYSLSSVVTV PSSSLGTKTY |
| | | TCNVDHKPSN TKVDKRVESK Y +/- GPPCPPCPA (or |
| | | GPPCPSCPA) +/- PEAAGGPSVFL |
| Galcanezumab | Light/ | DIQMTQSPSS LSASVGDRVT ITCRASKDIS KYLNWYQQKP |
| | SEQ ID | GKAPKLLIYY TSGYHSGVPS RFSGSGSGTD FTLTISSLQP |
| | NO: 30 | EDFATYYCQQ GDALPPTFGG GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Sevacizumab | Heavy/ | EVQLVESGGG LVKPGGSLRL SCAASGFSFS NNDVMCWVRQ |
| | SEQ ID | APGKGLEWIG CIMTTDVVTE YANWAKSRFT VSRDSAKNSV |
| | NO: 31 | YLQMNSLRAE DTAVYFCARD SVGSPLMSFD LWGPGTLVTV |
| | | SSASTKGPSV FPLAPSSKST SGGTAALGCL VKDYFPEPVT |
| | | VSWNSGALTS GVHTFPAVLQ SSGLYSLSSV VTVPSSSLGT |
| | | QTYICNVNHK PSNTKVDKKV EPKSCD +/- KTHT (or |
| | | KTHL) +/- CPPCPA +/- PELLGGPSVFL |
| Sevacizumab | Light/ | DIQMTQSPSS LSASVGDRVT INCQASQSIY NNNELSWYQQ |
| | SEQ ID | KPGKPPKLLI YRASTLASGV PSRFSGSGSG TDFTLTISSL |
| | NO: 32 | QPEDVATYYC GGYKSYSNDG NGFGGGTKVE IKRTVAAPSV |
| | | FIFPPSDEQL KSGTASVVCL LNNFYPREAK VQWKVDNALQ |
| | | SGNSQESVTE QDSKDSTYSL SSTLTLSKAD YEKHKVYACE |
| | | VTHQGLSSPV TKSFNRGEC |
| LKA-651 NVS2 | Heavy/ | EVQLVQSGAE VKKPGESLKI SCKGSGYSFT SYWIGWVRQM |
| | SEQ ID | PGKGLEWMGW IDPYRSEIRY SPSFQGQVTI SADKSISTAY |
| | NO: 33 | LQWSSLKASD TAMYYCARVS SEPFDSWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKRVEPKSC D +/- KTHT (or KTHL) |
| | | CPPCPA +/- PELLGGPSVFL |
| LKA-651 NVS2 | Light/ | SYVLTQPPSV SVAPGKTARI TCSGDKLGDH YAYWYQQKPG |
| | SEQ ID | QAPVLVIYDD SKRPSGIPER FSGSNSGNTA TLTISRVEAG |
| | NO: 34 | DEADYYCATW TFEGDYVFGG GTKLTVLGQP KAAPSVTLFP |
| | | PSSEELQANK ATLVCLISDF YPGAVTVAWK ADSSPVKAGV |
| | | ETTTPSKQSN NKYAASSYLS LTPEQWKSHR SYSCQVTHEG |
| | | STVEKTVAPT ECS |
| LKA-651 NVS3 | Heavy/ | QVQLQQSGPG LVKPSQTLSL TCAISGDSVS SNTAAWNWIR |
| | SEQ ID | QSPSRGLEWL GVIYYRSKWY NDYAVSVKSR ITINPDTSKN |
| | NO: 35 | QFSLQLNSVT PEDTAVYYCA RSVPGGDPGL EHAFAYWGRG |
| | | TLVTVSSAST KGPSVFPLAP SSKSTSGGTA ALGCLVKDYF |
| | | PEPVTVSWNS GALTSGVHTF PAVLQSSGLY SLSSVVTVPS |
| | | SSLGTQTYIC NVNHKPSNTK VDKRVEPKSC D +/- KTHT |
| | | (or KTHL) +/- CPPCPA +/- PELLGGPSVFL |
| LKA-651 NVS3 | Light/ | SYVLTQPPSV SVAPGKTARI TCSGDNLGTY YVEWYQQKPG |
| | SEQ ID | QAPVLVIYDD SDRPSGIPER FSGSNSGNTA TLTISRVEAG |
| | NO: 36 | DEADYYCASF ASWSDSVFGG GTKLTVLGQP KAAPSVTLFP |
| | | PSSEELQANK ATLVCLISDF YPGAVTVAWK ADSSPVKAGV |
| | | ETTTPSKQSN NKYAASSYLS LTPEQWKSHR SYSCQVTHEGS |
| | | TVEKTVAPTE CS |
| Ascrinvacumab | Heavy/ | QVQLQESGPG LVKPSQTLSL TCTVSGGSIS SGEYYWNWIR |
| | SEQ ID | QHPGKGLEWI GYIYYSGSTY YNPSLKSRVT ISVDTSKNQF |
| | NO: 37 | SLKLSSVTAA DTAVYYCARE SVAGFDYWGQ GTLVTVSSAS |
| | | TKGPSVFPLA PCSRSTSEST AALGCLVKDY FPEPVTVSWN |
| | | SGALTSGVHT FPAVLQSSGL YSLSSVVTVP SSNFGTQTYT |
| | | CNVDHKPSNT KVDKTVERKC CVE +/- CPPCPA +/- PPVAG |
| Ascrinvacumab | Light/ | EIVLTQSPGT LSLSPGERAT LSCRASQSVS SSYLAWYQQK |
| | SEQ ID | PGQAPRLLIY GTSSRATGIP DRFSGSGSGT DFTLTISRLE |
| | NO: 38 | PEDFAVYYCQ QYGSSPITFG QGTRLEIKRT VAAPSVFIFP |
| | | PSDEQLKSGT ASVVCLLNNF YPREAKVQWK VDNALQSGN |
| | | SQESVTEQDS KDSTYSLSST LTLSKADYEK HKVYACEVTH |
| | | QGLSSPVTKS FNRGEC |
| Tesidolumab | Heavy/ | EVQLVQSGAE VKKPGSSVKV SCKASGGTFS SYAISWVRQA |
| | SEQ ID | PGQGLEWMGG IGPFFGTANY AQKFQGRVTI TADESTSTAY |
| | NO: 39 | MELSSLRSED TAVYYCARDT PYFDYWGQGT LVTVSSASTK |
| | | GPSVFPLAPS SKSTSGGTAA LGCLVKDYFP EPVTVSWNSG |
| | | ALTSGVHTFP AVLQSSGLYS LSSVVTVPSS SLGTQTYICN |
| | | VNHKPSNTKV DKRVEPKSCD +/- KTHT |
| | | (or KTHL) +/- CPPCPA +/- PEAAGGPSVFL |
| Tesidolumab | Light/ | SYELTQPLSV SVALGQTARI TCSGDSIPNY YVYWYQQKPG |
| | SEQ ID | QAPVLVIYDD SNRPSGIPER FSGSNSGNTA TLTISRAQAG |
| | NO: 40 | DEADYYCQSF DSSLNAEVFG GGTKLTVLGQ PKAAPSVTLF |
| | | PPSSEELQAN KATLVCLISD FYPGAVTVAW KADSSPVKAG |
| | | VETTTPSKQS NNKYAASSYL SLTPEQWKSH RSYSCQVTHE |
| | | GSTVEKTVAP TECS |
| Carotuximab | Heavy/ | EVKLEESGGG LVQPGGSMKL SCAASGFTFS DAWMDWVRQS |
| | SEQ ID | PEKGLEWVAE IRSKASNHAT YYAESVKGRF TISRDDSKSS |
| | NO: 41 | VYLQMNSLRA EDTGIYYCTR WRRFFDSWGQ GTTLTVSSAS |
| | | TKGPSVFPLA PSSKSTSGGT AALGCLVKDY FPEPVTVSWN |
| | | SGALTSGVHT FPAVLQSSGL YSLSSVVTVP SSSLGTQTYI |
| | | CNVNHKPSNT KVDKKVEPKS CD +/- KTHT (or |
| | | KTHL) +/- CPPCPA +/- PELLGGPSVFL |
| Carotuximab | Light/ | QIVLSQSPAI LSASPGEKVT MTCRASSSVS YMHWYQQKPG |
| | SEQ ID | SSPKPWIYAT SNLASGVPVR FSGSGSGTSY SLTISRVEAE |
| | NO: 42 | DAATYYCQQW SSNPLTFGAG TKLELKRTVA APSVFIFPPS |
| | | DEQLKSGTAS VVCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| ANX-007 | Heavy/ | QVQLVQSGAE LKKPGASVKV SCKSSGYHFT SYWMHWVKQA |
| | SEQ ID | PGQGLEWIGV IHPNSGSINY NEKFESRVTI TVDKSTSTAY |
| | NO: 43 | MELSSLRSED TAVYYCAGER DSTEVLPMDY WGQGTTVTVS |
| | | SASTKGPSVF PLAPSSKSTS GGTAALGCLV KDYFPEPVTV |
| | | SWNSGALTSG VHTFPAVLQS SGLYSLSSVV TVPSSSLGTQ |
| | | TYICNVNHKP SNTKVDKRVE PKSCD +/- KTHT (or KTHL) +/- |
| | | CPPCPA +/- PELLGGPSVFL |
| ANX-007 | Light/ | DVQITQSPSS LSASLGERAT INCRASKSIN KYLAWYQQKP |
| | SEQ ID | GKAPKLLIYS GSTLQSGIPA RFSGSGSGTD FTLTISSLEP |
| | NO: 44 | EDFAMYYCQQ HNEYPLTFGQ GTKLEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| 363 | | LSSPVTKSFN RGEC |
| Adalimumab | Heavy/ | EVQLVESGGG LVQPGRSLRL SCAASGFTFD DYAMHWVRQA |
| | SEQ ID | PGKGLEWVSA ITWNSGHIDY ADSVEGRFTI SRDNAKNSLY |
| | NO: 45 | LQMNSLRAED TAVYYCAKVS YLSTASSLDY WGQGTLVTVS |
| | | SASTKGPSVF PLAPSSKSTS GGTAALGCLV KDYFPEPVTV |
| | | SWNSGALTSG VHTFPAVLQS SGLYSLSSVV TVPSSSLGTQ |
| | | TYICNVNHKP SNTKVDKKVE PKSCD +/- KTHT (or KTHL) +/- |
| | | CPPCPA +/- PELLGGPSVFL |
| Adalimumab | Light/ | DIQMTQSPSS LSASVGDRVT ITCRASQGIR NYLAWYQQKP |
| | SEQ ID | GKAPKLLIYA ASTLQSGVPS RFSGSGSGTD FTLTISSLQP |
| | NO: 46 | EDVATYYCQR YNRAPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Infliximab | Heavy/ | EVKLEESGGG LVQPGGSMKL SCVASGFIFS NHWMNWVRQS |
| | SEQ ID | PEKGLEWVAE IRSKSINSAT HYAESVKGRF TISRDDSKSA |
| | NO: 47 | VYLQMTDLRT EDTGVYYCSR NYYGSTYDYW GQGTTLTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCD +/- KTHT (or |
| | | KTHL)+/- CPPCPA +/- PELLGGPSVFL |
| Infliximab | Light/ | DILLTQSPAI LSVSPGERVS FSCRASQFVG SSIHWYQQRT |
| | SEQ ID | NGSPRLLIKY ASESMSGIPS RFSGSGSGTD FTLSINTVES |
| | NO: 48 | EDIADYYCQQ SHSWPFTFGS GTNLEVKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Golimumab | Heavy/ | SKLQVQLVES GGGVVQPGRS LRLSCAASGF IFSSYAMHWV |
| | SEQ ID | RQAPGNGLEW VAFMSYDGSN KKYADSVKGR FTISRDNSKN |
| | NO: 49 | TLYLQMNSLR AEDTAVYYCA RDRGIAAGGN YYYYGMDVWG |
| | | QGTTVTVSSA STKGPSVFPL APSSKSTSGG TAALGCLVKD |
| | | YFPEPVTVSW NSGALTSGVH TFPAVLQSSG LYSLSSVVTV |
| | | PSSSLGTQTY ICNVNHKPSN TKVDKKVEPK SCD +/- |
| | | KTHT (or KTHL) +/- CPPCPA +/- PELLGGPSVFL |
| Golimumab | Light/ | AGSEIVLTQS PATLSLSPGE RATLSCRASQ SVYSYLAWYQ |
| | SEQ ID | QKPGQAPRLL IYDASNRATG IPARFSGSGS GTDFTLTISS |
| | NO: 50 | LEPEDFAVYY CQQRSNWPPF TFGPGTKVDI KRTVAAPSVF |
| | | IFPPSDEQLK SGTASVVCLL NNFYPREAKV QWKVDNALQS |
| | | GNSQESVTEQ DSKDSTYSLS STLTLSKADY EKHKVYACEV |
| | | THQGLSSPVT KSFNRGEC |
| Elezanumab | Heavy/ | EVQLVQSGAE VKKPGASVKV SCKASGYTFT SHGISWVRQA |
| | SEQ ID | PGQGLDWMGW ISPYSGNTNY AQKLQGRVTM TTDTSTSTAY |
| | NO: 51 | MELSSLRSED TAVYYCARVG SGPYYYMDVW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCD +/- KTHT (or |
| | | KTHL)+/- CPPCPA +/- PEAAGGPSVFL |
| Elezanumab | Light/ | QSALTQPRSV SGSPGQSVTI SCTGTSSSVG DSIYVSWYQQ |
| | SEQ ID | HPGKAPKLML YDVTKRPSGV PDRFSGSKSG NTASLTISGL |
| | NO: 52 | QAEDEADYYC YSYAGTDTLF GGGTKVTVLG QPKAAPSVTL |
| | | FPPSSEELQA NKATLVCLIS DFYPGAVTVA WKADSSPVKA |
| | | GVETTTPSKQ SNNKYAASSY LSLTPEQWKS HRSYSCQVTH |
| | | EGSTVEKTVA PTECS |
| NI-301 | Heavy/ | QVQLQESGPG LVKPSETLSL TCSVSGGSII SRSSYWGWIR |
| | SEQ ID | QPPGKGLEWI GGIYHSGNTY DNPSLKSRLT MSVDTSKNQF |
| | NO: 53 | SLNLRSVTAA DTAVYYCARI VPGGDAFDIW GQGTMVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKRVEP KSCD +/- KTHT (or KTHL)+/- |
| | | CPPCPA +/- PELLGGPSVFL |
| NI-301 | Light/ | DIQMTQSPSS LSASVGDRVT ACRASQSVGT YLNWYQQKRG |
| | SEQ ID | KAPKLLIFAA SSLQSGVPSR FSGSGSGTDF TLTISSLQPE |
| | NO: 54 | DFATYYCQQS YSSPPTFGQG TKVEIKRTVA APSVFIFPPS |
| | | DEQLKSGTAS VVCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| PRX-004 | Heavy/ | EVQLVESGGG LIQPGGSLRL SCAASGFTFS NYAMSWVRQA |
| | SEQ ID | PGKGLEWVSS ISSGGSTYYP DSVKGRFTIS RDNSKNTLYL |
| | NO: 55 | QMNSLRAEDT AVYYCARYYY GQYFDFWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKRVEPKSC D +/- |
| | | KTHT (or KTHL) +/- CPPCPA +/- PELLGGPSVFL |
| PRX-004 | Light/ | DIVMTQSPSS LSASVGDRVT ITCKASQDVS TTVAWYQQKP |
| | SEQ ID | GKAPKLLIYS ASYRSTGVPS RFSGSGSGTD FTLTISSLQP |
| | NO: 56 | EDFAVYYCQQ HYSTPLTFGG GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Pamrevlumab | Heavy/ | EVQLVQSGAE VKKPGSSVKV SCKASGGTFS SYAISWVRQA |
| | SEQ ID | PGQGLEWMGG IGPFFGTANY AQKFQGRVTI TADESTSTAY |
| | NO: 57 | MELSSLRSED TAVYYCARDT PYFDYWGQGT LVTVSSASTK |
| | | GPSVFPLAPS SKSTSGGTAA LGCLVKDYFP EPVTVSWNSG |
| | | ALTSGVHTFP AVLQSSGLYS LSSVVTVPSS SLGTQTYICN |
| | | VNHKPSNTKV DKRVEPKSCD +/- KTHT (or KTHL)+/- |
| | | CPPCPA +/- PEAAGGPSVFL |
| Pamrevlumab | Light/ | SYELTQPLSV SVALGQTARI TCSGDSIPNY YVYWYQQK2G |
| | SEQ ID | QAPVLVIYDD SNRPSGIPER FSGSNSGNTA TLTISRAQAG |
| | NO: 58 | DEADYYCQSF DSSLNAEVFG GGTKLTVLGQ PKAAPSVTLF |
| | | PPSSEELQAN KATLVCLISD FYPGAVTVAW KADSSPVKAG |
| | | VETTTPSKQS NNKYAASSYL SLTPEQWKSH RSYSCQVTHE |
| | | GSTVEKTVAP TECS |
| Satralizumab | Heavy/ | QVQLQESGPG LVKPSETLSL TCAVSGHSIS HDHAWSWVRQ |
| | SEQ ID | PPGEGLEWIG FISYSGITNY NPSLQGRVTI SRDNSKNTLY |
| | NO: 59 | LQMNSLRAED TAVYYCARSL ARTTAMDYWG EGTLVTVSSA |
| | | STKGPSVFPL APSSKSTSGG TAALGCLVKD YFPEPVTVSW |
| | | NSGALTSGVH TFPAVLQSSG LYSLSSVVTV PSSNFGTQTY |
| | | TCNVDHKPSN TKVDKTVERK SCVE +/- CPPCPA +/- |
| | | PPVAG |
| Satralizumab | Light/ | DIQMTQSPSS LSASVGDSVT ITCQASTDIS SHLNWYQQKP |
| | SEQ ID | GKAPELLIYY GSHLLSGVPS RFSGSGSGTD FTFTISSLEA |
| | NO: 60 | EDAATYYCGQ GNRLPYTFGQ GTKVEIERTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Sarilumab | Heavy/ | EVQLVESGGG LVQPGRSLRL SCAASRFTFD DYAMHWVRQA |
| | SEQ ID | PGKGLEWVSG ISWNSGRIGY ADSVKGRFTI SRDNAENSLF |
| | NO: 61 | LQMNGLRAED TALYYCAKGR DSFDIWGQGT MVTVSSASTK |
| | | GPSVFPLAPS SKSTSGGTAA LGCLVKDYFP EPVTVSWNSG |
| | | ALTSGVHTFP AVLQSSGLYS LSSVVTVPSS SLGTQTYICN |
| | | VNHKPSNTKV DKKVEPKSCD +/- KTHT (or KTHL)+/- |
| | | CPPCPA +/- PELLGGPSVFL |
| Sarilumab | Light/ | DIQMTQSPSS VSASVGDRVT ITCRASQGIS SWLAWYQQKP |
| | SEQ ID | GKAPKLLIYG ASSLESGVPS RFSGSGSGTD FTLTISSLQP |
| | NO: 62 | EDFASYYCQQ ANSFPYTFGQ GTKLEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Inebilizumab | Heavy/ | EVQLVESGGG LVQPGGSLRL SCAASGFTFS SSWMNWVRQA |
| | SEQ ID | PGKGLEWVGR IYPGDGDTNY NVKFKGRFTI SRDDSKNSLY |
| | NO: 63 | LQMNSLKTED TAVYYCARSG FITTVRDFDY WGQGTLVTVS |
| | | SASTKGPSVF PLAPSSKSTS GGTAALGCLV KDYFPEPVTV |
| | | SWNSGALTSG VHTFPAVLQS SGLYSLSSVV TVPSSSLGTQ |
| | | TYICNVNHKP SNTKVDKRVE PKSCD +/- KTHT (or |
| | | KTHL)+/- CPPCPA +/- PELLGGPSVFL |
| Inebilizumab | Light/ | EIVLTQSPD FQSVTPKEK VTITCRASE SVDTFGISF MNWFQQKPD |
| | SEQ ID | QSPKLLIHE ASNQGSGVP SRFSGSGSG TDFTLTINS LEAEDAATY |
| | NO: 64 | YCQQSKEVP FTFGGGTKV EIKRTVAAP SVFIFPPSD EQLKSGTAS |
| | | VVCLLNNFY PREAKVQWK VDNALQSGN SQESVTEQD SKDSTYSLS |
| | | STLTLSKAD YEKHKVYAC EVTHQGLSS PVTKSFNRGEC |
| Etrolizumab | Heavy/ | EVQLVESGGG LVQPGGSLRL SCAASGFFIT NNYWGWVRQA |
| | SEQ ID | PGKGLEWVGY ISYSGSTSYN PSLKSRFTIS RDTSKNTFYL |
| | NO: 65 | QMNSLRAEDT AVYYCARTGS SGYFDFWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSCD +/- KTHT (or KTHL) +/- |
| | | CPPCPA +/- PELLGGPSVFL |
| Etrolizumab | Light/ | DIQMTQSPSS LSASVGDRVT ITCRASESVD DLLHWYQQKP |
| | SEQ ID | GKAPKLLIKY ASQSISGVPS RFSGSGSGTD FTLTISSLQP |
| | NO: 66 | EDFATYYCQQ GNSLPNTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Romosozumab | Heavy/ | EVQLVQSGAE VKKPGASVKV SCKASGYTFT DYNMHWVRQA |
| | SEQ ID | PGQGLEWMGE INPNSGGAGY NQKFKGRVTM TTDTSTSTAY |
| | NO: 67 | MELRSLRSDD TAVYYCARLG YDDIYDDWYF DVWGQGTTVT |
| | | VSSASTKGPS VFPLAPCSRS TSESTAALGC LVKDYFPEPV |
| | | TVSWNSGALT SGVHTFPAVL QSSGLYSLSS WTVPSSNFG |
| | | TQTYTCNVDH KPSNTKVDKT VERKCCVE +/- CPPCPA+/- PPVAG |
| Romosozumab | Light/ | DIQMTQSPSS LSASVGDRVT ITCRASQDIS NYLNWYQQKP |
| | SEQ ID | GKAPKLLIYY TSRLLSGVPS RFSGSGSGTD FTLTISSLQP |
| | NO: 68 | EDFATYYCQQ GDTLPYTFGG GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Lanadelumab | Heavy/ | EVQLLESGGG LVQPGGSLRL SCAASGFTFS HYIMMWVRQA |
| | SEQ ID | PGKGLEWVSG IYSSGGITVY ADSVKGRFTI SRDNSKNTLY |
| | NO: 69 | LQMNSLRAED TAVYYCAYRR IGVPRRDEFD IWGQGTMVTV |
| | | SSASTKGPSV FPLAPSSKST SGGTAALGCL VKDYFPEPVT |
| | | VSWNSGALTS GVHTFPAVLQ SSGLYSLSSV VTVPSSSLGT |
| | | QTYICNVNHK PSNTKVDKRV EPKSCD +/- KTHT (or KTHL) +/- |
| | | CPPCPA +/- PELLGGPSVFL |
| Lanadelumab | Light/ | DIQMTQSPST LSASVGDRVT ITCRASQSIS SWLAWYQQKP |
| | SEQ ID | GKAPKLLIYK ASTLESGVPS RFSGSGSGTE FTLTISSLQP |
| | NO: 70 | DDFATYYCQQ YNTYWTFGQG TKVEIKRTVA APSVFIFPPS |
| | | DEQLKSGTAS WCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| Siltuximab | Heavy/ | EVQLVESGGK LLKPGGSLKL SCAASGFTFS SFAMSWFRQS |
| | SEQ ID | PEKRLEWVAE ISSGGSYTYY PDTVTGRFTI SRDNAKNTLY |
| | NO: 331 | LEMSSLRSED TAMYYCARGL WGYYALDYWG QGTSVTVSSA |
| | | STKGPSVFPL APSSKSTSGG TAALGCLVKD YFPEPVTVSW |
| | | NSGALTSGVH TFPAVLQSSG LYSLSSVVTV PSSSLGTQTY |
| | | ICNVNHKPSN TKVDKKVEPK SCD +/- KTHT (or KTHL) +/- |
| | | CPPCPA +/- PELLGGPSVFL |
| Siltuximab | Heavy/ | QIVLIQSPAI MSASPGEKVT MTCSASSSVS YMYWYQQKPG |
| | SEQ ID | SSPRLLIYDT SNLASGVPVR FSGSGSGTSY SLTISRMEAE |
| | NO: 332 | DAATYYCQQW SGYPYTFGGG TKLEIKRTVA APSVFIFPPS |
| | | DEQLKSGTAS VVCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| Clazakizumab | Heavy/ | EVQLVESGGG LVQPGGSLRL SCAASGFSLS NYYVTWVRQA |
| | SEQ ID | PGKGLEWVGI IYGSDETAYA TSAIGRFTIS RDNSKNTLYL |
| | NO: 333 | QMNSLRAEDT AVYYCARDDS SDWDAKFNLW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKRVEP KSCD +/- KTHT (or KTHL) +/- |
| | | CPPCPA +/- PELLGGPSVFL |
| Clazakizumab | Light/ | AIQMTQSPSS LSASVGDRVT ITCQASQSIN NELSWYQQKP |
| | SEQ ID | GKAPKLLIYR ASTLASGVPS RFSGSGSGTD FTLTISSLQP |
| | NO: 334 | DDFATYYCQQ GYSLRNIDNA FGGGTKVEIK RTVAAPSVFI |
| | | FPPSDEQLKS GTASVVCLLN NFYPREAKVQ WKVDNALQSG |
| | | NSQESVTEQD SKDSTYSLSS TLTLSKADYE KHKVYACEVT |
| | | HQGLSSPVTK SFNRGEC |
| Sirukumab | Heavy/ | EVQLVESGGG LVQPGGSLRL SCAASGFTFS PFAMSWVRQA |
| | SEQ ID | PGKGLEWVAK ISPGGSWTYY SDTVTGRFTI SRDNAKNSLY |
| | NO: 335 | LQMNSLRAED TAVYYCARQL WGYYALDIWG QGTTVTVSSA |
| | | STKGPSVFPL APSSKSTSGG TAALGCLVKD YFPEPVTVSW |
| | | NSGALTSGVH TFPAVLQSSG LYSLSSVVTV PSSSLGTQTY |
| | | ICNVNHKPSN TKVDKKVEPK SCD +-/ KTHT (or KTHL) +/- |
| | | CPPCPA +-/ PELLGGPSVFL |
| Sirukumab | Light/ | EIVLTQSPAT LSLSPGERAT LSCSASISVS YMYWYQQKPG |
| | SEQ ID | QAPRLLIYDM SNLASGIPAR FSGSGSGTDF TLTISSLEPE |
| | NO: 336 | DFAVYYCMQW SGYPYTFGGG TKVEIKRTVA APSVFIFPPS |
| | | DEQLKSGTAS VVCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| Olokizumab | Heavy/ | EVQLVESGGG LVQPGGSLRL SCAASGFNFN DYFMNWVRQA |
| | SEQ ID | PGKGLEWVAQ MRNKNYQYGT YYAESLEGRF TISRDDSKNS |
| | NO: 337 | LYLQMNSLKT EDTAVYYCAR ESYYGFTSYW GQGTLVTVSS |
| | | ASTKGPSVFP LAPCSRSTSE STAALGCLVK DYFPEPVTVS |
| | | WNSGALTSGV HTFPAVLQSS GLYSLSSVVT VPSSSLGTKT |
| | | YTCNVDHKPS NTKVDKRVES KY +/- GPPCPPCPA (or |
| | | GPPCPSCPA) +/- PEFLGGPSVFL |
| Olokizumab | Light/ | DIQMTQSPSS LSASVGDRVT ITCQASQDIG ISLSWYQQKP |
| | SEQ ID | GKAPKLLIYN ANNLADGVPS RFSGSGSGTD FTLTISSLQP |
| | NO: 338 | EDFATYYCLQ HNSAPYTFGQ GTKLEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Gerilimzumab | Heavy/ | EVQLQESGPG LVKPSQTLSL TCTVSGGSIT SRYYAWSWIR |
| | SEQ ID | QPPGKGLEWI GVIDYDGDTY YSPSLKSRVS ISWDTSKNQF |
| | NO: 339 | SLKLSSVTPA DTAVYYCARD PDVVTGFHYD YWGQGTMVTV |
| | | SSASTKGPSV FPLAPSSKST SGGTAALGCL VKDYFPEPVT |
| | | VSWNSGALTS GVHTFPAVLQ SSGLYSLSSV VTVPSSSLGT |
| | | QTYICNVNHK PSNTKVDKKV EPKSCD +/- KTHT (or KTHL) +/- |
| | | CPPCPA +-/ PELLGGPSVFL |
| Gerilimzumab | Light/ | QSALTQPPSV SGTPGQSVTI SCAGANNDIG TYAYVSWYQQ |
| | SEQ ID | LPGTAPKLMI YKVTTRASGI PDRFSGSKSG NTASLTISGL |
| | NO: 340 | QAEDEADYYC ASYRNFNNAV FGTGTKLTVL GQPKAAPSVT |
| | | LFPPSSEELQ ANKATLVCLI SDFYPGAVTV AWKADSSPVK |
| | | AGVETTTPSK QSNNKYAASS YLSLTPEQWK SHRSYSCQVT |
| | | HEGSTVEKTV APTECS |
| Tocilizumab | Heavy/ | QVQLQESGPG LVRPSQTLSL TCTVSGYSIT SDHAWSWVRQ |
| | SEQ ID | PPGRGLEWIG YISYSGITTY NPSLKSRVTM LRDTSKNQFS |
| | NO: 341 | LRLSSVTAAD TAVYYCARSL ARTTAMDYWG QGSLVTVSSA |
| | | STKGPSVFPL APSSKSTSGG TAALGCLVKD YFPEPVTVSW |
| | | NSGALTSGVH TFPAVLQSSG LYSLSSVVTV PSSSLGTQTY |
| | | ICNVNHKPSN TKVDKKVEPK SCD +/- KTHT (or KTHL) +/- |
| | | CPPCPA +/- PELLGGPSVFL |
| Tocilizumab | Light/ | DIQMTQSPSS LSASVGDRVT ITCRASQDIS SYLNWYQQKP |
| | SEQ ID | GKAPKLLIYY TSRLHSGVPS RFSGSGSGTD FTFTISSLQP |
| | NO: 342 | EDIATYYCQQ GNTLPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Lecanemab | Heavy/ | EVQLVESGGG LVQPGGSLRL SCSASGFTFS SFGMHWVRQA |
| | SEQ ID | PGKGLEWVAY ISSGSSTIYY GDTVKGRFTI SRDNAKNSLF |
| | NO: 360 | LQMSSLRAED TAVYYCAREG GYYYGRSYYT MDYWGQGTTV |
| | | TVSSASTKGP SVFPLAPSSK STSGGTAALG CLVKDYFPEP |
| | | VTVSWNSGAL TSGVHTFPAV LQSSGLYSLS SVVTVPSSSL |
| | | GTQTYICNVN HKPSNTKVDK RVEPKSCD +/- KTHT +/- CPPCPA |
| | | +/- PELLGGPSVFL |
| Lecanemab | Light/ | DVVMTQSPLS LPVTPGAPAS ISCRSSQSIV HSNGNTYLEW |
| | SEQ ID | YLQKPGQSPK LLIYKVSNRF SGVPDRFSGS GSGTDFTLRI |
| | NO: 361 | SRVEAEDVGI YYCFQGSHVP PTFGPGTKLE IKRTVAAPSV |
| | | FIFPPSDEQL KSGTASVVCL LNNFYPREAK VQWKVDNALQ |
| | | SGNSQESVTE QDSKDSTYSL SSTLTLSKAD YEKHKVYACE |
| | | VTHQGLSSPV TKSFNRGEC |
| Ravulizumab | Heavy/ | QVQLVQSGA3 VKKPGASVKV SCKASGHIFS NYWIQWVRQA |
| | SEQ ID | PGQGLEWMGE ILPGSGHTEY TENFKDRVTM TRDTSTSTVY |
| | NO: 362 | MELSSLRSED TAVYYCARYE FGSSPNWYFD VWGQGTLVTV |
| | | SSASTKGESV FPLAPCSRST SESTAALGCL VKDYFPEPVT |
| | | VSWNSGALTS GVHTFPAVLQ SSGLYSLSSV VTVPSSNFGT |
| | | QTYTCNVDHK PSNTKVDKTV ERKCCVE +/- CPPCPA+/- PPVAG |
| Ravulizumab | Light/ | DIQMTQSPSS LSASVGDRVT ITCGASENIY GALNWYQQKP |
| | SEQ ID | GKAPKLLIYG ATNLADGVPS RFSGSGSGTD FTITISSLQP |
| | NO: 363 | EDFATYYCQN VLNTPLTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Benralizumab | Heavy/ | EVQLVQSGAE VKKPGASVKV SCKASGYTFT SYVIHWVRQR |
| | SEQ ID | PGQGLAWMGY INPYNDGTKY NERFKGKVTI TSDRSTSTVY |
| | NO: 364 | MELSSLRSED TAVYLCGREG IRYYGLLGDY WGQGTLVTVS |
| | | SASTKGPSVF PLAPSSKSTS GGTAALGCLV KDYFPEPVTV |
| | | SWNSGALTSG VHTFPAVLQS SGLYSLSSVV TVPSSSLGTQ |
| | | TYICNVNHKP SNTKVDKKV EPKSCD +/- KTHT (or |
| | | KTHL) +/- CPPCPA +/- PELLGGPSVFL |
| Benralizumab | Light/ | DIQMTQSPSS LSASVGDRVT ITCGTSEDII NYLNWYQQKP |
| | SEQ ID | GKAPKLLIYH TSRLQSGVPS RFSGSGSGTD FTLTISSLQP |
| | NO: 365 | EDFATYYCQQ GYTLPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Reslizumab | Heavy/ | EVQLVESGGG LVQPGGSLRL SCAVSGLSLT SNSVNWIRQA |
| | SEQ ID | PGKGLEWVGL IWSNGDTDYN SAIKSRFTIS RDTSKSTVYL |
| | NO: 366 | QMNSLRAEDT AVYYCAREYY GYFDYWGQGT LVTVSSASTK |
| | | GPSVFPLAPC SRSTSESTAA LGCLVKDYFP EPVTVSWNSG |
| | | ALTSGVHTFP AVLQSSGLYS LSSVVTVPSS SLGTKTYTCN |
| | | VDHKPSNTKV DKRV ESKY +/- GPPCPPCPA (or GPPCP |
| | | SCPA) +/- PEFLGGPSVFL |
| Reslizumab | Light/ | DIQMTQSPSS LSASVGDRVT ITCLASEGIS SYLAWYQQKP |
| | SEQ ID | GKAPKLLIYG ANSLQTGVPS RFSGSGSATD YTLTISSLQP |
| | NO: 367 | EDFATYYCQQ SYKFPNTFGQ GTKVEVKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Tralokinumab | Heavy/ | QVQLVQSGAE VKKPGASVKV SCKASGYTFT NYGLSWVRQA |
| | SEQ ID | PGQGLEWMGW ISANNGDTNY GQEFQGRVTM TTDTSTSTAY |
| | NO: 368 | MELRSLRSDD TAVYYCARDS SSSWARWFFD LWGRGTLVTV |
| | | SSASTKGPSV FPLAPCSRST SESTAALGCL VKDYFPEPVT |
| | | VSWNSGALTS GVHTFPAVLQ SSGLYSLSSV VTVPSSSLGT |
| | | 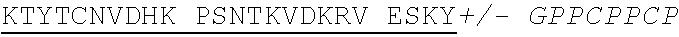 |
| | | 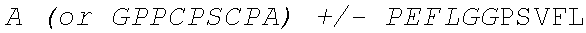 |
| Tralokinumab | Light/ | SYVLTQPPSV SVAPGKTARI TCGGNIIGSK LVHWYQQKPG |
| | SEQ ID | QAPVLVIYDD GDRPSGIPER FSGSNSGNTA TLTISRVEAG |
| | NO:369 | DEADYYCQVW DTGSDPVVFG GGTKLTVLGQ PKAAPSVTLF |
| | | PPSSEELQAN KATLVCLISD FYPGAVTVAW KADSSPVKAG |
| | | VETTTPSKQS NNKYAASSYL SLTPEQWKSH RSYSCQVTHE |
| | | GSTVEKTVAP TECS |
| Nemolizumab | Heavy/ | QVQLVQSGAE VKKPGASVKV SCKASGYTFT GYIMNWVRQA |
| | SEQ ID | PGQGLEWMGL INPYNGGTDY NPQFQDRVTI TADKSTSTAY |
| | NO: 370 | MELSSLRSED TAVYYCARDG YDDGPYTLET WGQGTLVTVS |
| | | SASTKGPSVF PLAPSSKSTS GGTAALGCLV KDYFPEPVTV |
| | | SWNSGALTSG VHTFPAVLQS SGLYSLSSVV TVPSSNFGTQ |
| | | TYTCNVDHKP SNTKVDKTVERK SCVE +/- CPPCPA +/- |
| | | PPVAG |
| Nemolizumab | Light/ | DIQMTQSPSS LSASVGDRVT ITCQASEDIY SFVAWYQQKP |
| | SEQ ID | GKAPKLLIYN AQTEAQGVPS RFSGSGSGTD FTLTISSLQP |
| | NO: 371 | EDFATYYCQH HYDSPLTFGG GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Omalizumab | Heavy/ | EVQLVESGGG LVQPGGSLRL SCAVSGYSIT SGYSWNWIRQ |
| | SEQ ID | APGKGLEWVA SITYDGSTNY ADSVKGRFTI SRDDSKNTFY |
| | NO: 372 | LQMNSLRAED TAVYYCARGS HYFGHWHFAV WGQGTLVTVS |
| | | SGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKA EPKSCD +/- KTHT (or |
| | | KTHL) +/- CPPCPA +/- PELLGGPSVFL |
| Omalizumab | Light/ | DIQLTQSPSS LSASVGDRVT ITCRASQSVD YDGDSYMNWY |
| | SEQ ID | QQKPGKAPKL LIYAASYLES GVPSRFSGSG SGTDFTLTIS |
| | NO: 373 | SLQPEDFATY YCQQSHEDPY TFGQGTKVEI KRTVAAPSVF |
| | | IFPPSDEQLK SGTASVVCLL NNFYPREAKV QWKVDNALQS |
| | | GNSQESVTEQ DSKDSTYSLS STLTLSKADY EKHKVYACEV |
| | | THQGLSSPVT KSFNRGEC |
| Tezepelumab | Heavy/ | QMQLVESGGG VVQPGRSLRL SCAASGFTFR TYGMHWVRQA |
| | SEQ ID | PGKGLEWVAV IWYDGSNKHY ADSVKGRFTI TRDNSKNTLN |
| | NO:374 | LQMNSLRAED TAVYYCARAP QWELVHEAFD IWGQGTMVTV |
| | | SSASTKGPSV FPLAPCSRST SESTAALGCL VKDYFPEPVT |
| | | VSWNSGALTS GVHTFPAVLQ SSGLYSLSSV VTVPSSNFGT |
| | | QTYTCNVDHK PSNTKVDKTV ERKCCVE +/- CPPCPA +/- |
| | | PPVAG |
| Tezepelumab | Light/ | SYVLTQPPSV SVAPGQTARI TCGGNNLGSK SVHWYQQKPG |
| | SEQ ID | QAPVLVVYDD SDRPSWIPER FSGSNSGNTA TLTISRGEAG |
| | NO: 375 | DEADYYCQVW DSSSDHVVFG GGTKLTVLGQ PKAAPSVTLF |
| | | PPSSEELQAN KATLVCLISD FYPGAVTVAW KADSSPVKAG |
| | | VETTTPSKQS NNKYAASSYL SLTPEQWKSH RSYSCQVTHE |
| | | GSTVEKTVAP TECS |

TABLE 6
Table of Fab Fragment Nucleic Acid Sequences

| | Chain/ | |
|---|---|---|
| | SEQ ID | |
| mAb | NO: | Sequence |
| Solanezumab | Heavy/ | GAGGTGCAGCTGGTGGAGAGCGGCGGCGGCCTGGTGCAGCCCGGCGGCAGCC |
| | SEQ ID | TGAGGCTGAGCTGCGCCGCCAGCGGCTTCACCTTCAGCAGGTACAGCATGAG |
| | NO: 71 | CTGGGTGAGGCAGGCCCCCGGCAAGGGCCTGGAGCTGGTGGCCCAGATCAAC |
| | | AGCGTGGGCAACAGCACCTACTACCCCGACACCGTGAAGGGCAGGTTCACCA |
| | | TCAGCAGGGACAACGCCAAGAACACCCTGTACCTGCAGATGAACAGCCTGAG |
| | | GGCCGAGGACACCGCCGTGTACTACTGCGCCAGCGGCGACTACTGGGGCCAG |
| | | GGCACCCTGGTGACCGTGAGCAGCGCCAGCACCAAGGGCCCCAGCGTGTTCC |
| | | CCCTGGCCCCCAGCAGCAAGAGCACCAGCGGCGGCACCGCCGCCCTGGGCTG |
| | | CCTGGTGAAGGACTACTTCCCCGAGCCCGTGACCGTGAGCTGGAACAGCGGC |
| | | GCCCTGACCAGCGGCGTGCACACCTTCCCCGCCGTGCTGCAGAGCAGCGGCC |
| | | TGTACAGCCTGAGCAGCGTGGTGACCGTGCCCAGCAGCAGCCTGGGCACCCA |
| | | GACCTACATCTGCAACGTGAACCACAAGCCCAGCAACACCAAGGTGGACAAG |
| | | AAGGTGGAGCCCAAGAGCTGCGAC +/− AAGACCCACACC (or |
| | | AAGACCCACCTG) +/− TGCCCCCCCTGCCCCGCC +/− |
| | | CCCGAGCTGCTGGGCGGCCCCAGCGTGTTCCTG |
| Solanezumab | Light/ | GACGTGGTGATGACCCAGAGCCCCCTGAGCCTGCCCGTGACCCTGGGCCAGC |
| | SEQ ID | CCGCCAGCATCAGCTGCAGGAGCAGCCAGAGCCTGATCTACAGCGACGGCAA |
| | NO: 72 | CGCCTACCTGCACTGGTTCCTGCAGAAGCCCGGCCAGAGCCCCAGGCTGCTG |
| | | ATCTACAAGGTGAGCAACAGGTTCAGCGGCGTGCCCGACAGGTTCAGCGGCA |
| | | GCGGCAGCGGCACCGACTTCACCCTGAAGATCAGCAGGGTGGAGGCCGAGGA |
| | | CGTGGGCGTGTACTACTGCAGCCAGAGCACCCACGTGCCCTGGACCTTCGGC |
| | | CAGGGCACCAAGGTGGAGATCAAGAGGACCGTGGCCGCCCCCAGCGTGTTCA |
| | | TCTTCCCCCCCAGCGACGAGCAGCTGAAGAGCGGCACCGCCAGCGTGGTGTG |
| | | CCTGCTGAACAACTTCTACCCCAGGGAGGCCAAGGTGCAGTGGAAGGTGGAC |
| | | AACGCCCTGCAGAGCGGCAACAGCCAGGAGAGCGTGACCGAGCAGGACAGCA |
| | | AGGACAGCACCTACAGCCTGAGCAGCACCCTGACCCTGAGCAAGGCCGACTA |
| | | CGAGAAGCACAAGGTGTACGCCTGCGAGGTGACCCACCAGGGCCTGAGCAGC |
| | | CCCGTGACCAAGAGCTTCAACAGGGGCGAGTGC |
| GSK933776 | Heavy/ | GAGGTGCAGCTGGTGGAGAGCGGCGGCGGCCTGGTGCAGCCCGGCGGCAGCC |
| | SEQ ID | TGAGGCTGAGCTGCGCC(GCC/GTG)AGCGGCTTCACCTTCAGCGACAACGG |
| | NO: 73 | CATGGCCTGGGTGAGGCAGGCCCCCGGCAAGGGCCTGGAGTGGGTGAGCTTC |
| | | ATCAGCAACCTGGCCTACAGCATCGACTACGCCGACACCGTGACCGGCAGGT |
| | | TCACCATCAGCAGGGACAACGCCAAGAACAGCCTGTACCTGCAGATGAACAG |
| | | CCTGAGGGCCGAGGACACCGCCGTGTACTACTGCGTGAGCGGCACCTGGTTC |
| | | GCCTACTGGGGCCAGGGCACCCTGGTGACCGTGAGCAGCGCCAGCACCAAGG |
| | | GCCCCAGCGTGTTCCCCCTGGCCCCCAGCAGCAAGAGCACCAGCGGCGGCAC |
| | | CGCCGCCCTGGGCTGCCTGGTGAAGGACTACTTCCCCGAGCCCGTGACCGTG |
| | | AGCTGGAACAGCGGCGCCCTGACCAGCGGCGTGCACACCTTCCCCGCCGTGC |
| | | TGCAGAGCAGCGGCCTGTACAGCCTGAGCAGCGTGGTGACCGTGCCCAGCAG |
| | | CAGCCTGGGCACCCAGACCTACATCTGCAACGTGAACCACAAGCCCAGCAAC |
| | | ACCAAGGTGGACAAGAAGGTGGAGCCCAAGAGCTGCGAC +/− AAGACCC |
| | | ACACC (or AAGACCCACCTG) +/− TGCCCCCCCTGCCCCGCC +/− |
| | | CCCGAGCTGGCCGGCGCCCCCAGCGTGTTCCTG |
| GSK933776 | Light/ | GACATCGTGATGACCCAGAGCCCCCTGAGCCTGCCCGTGACCCCCGGCGAGC |
| | SEQ ID | CCGCCAGCATCAGCTGCAGGGTGAGCCAGAGCCTGCTGCACAGCAACGGCTA |
| | NO: 74 | CACCTACCTGCACTGGTACCTGCAGAAGCCCGGCCAGAGCCCCCAGCTGCTG |
| | | ATCTACAAGGTGAGCAACAGGTTCAGCGGCGTGCCCGACAGGTTCAGCGGCA |
| | | GCGGCAGCGGCACCGACTTCACCCTGAAGATCAGCAGGGTGGAGGCCGAGGA |
| | | CGTGGGCGTGTACTACTGCAGCCAGACCAGGCACGTGCCCTACACCTTCGGC |
| | | GGCGGCACCAAGGTGGAGATCAAGAGGACCGTGGCCGCCCCCAGCGTGTTCA |
| | | TCTTCCCCCCCAGCGACGAGCAGCTGAAGAGCGGCACCGCCAGCGTGGTGTG |
| | | CCTGCTGAACAACTTCTACCCCAGGGAGGCCAAGGTGCAGTGGAAGGTGGAC |
| | | AACGCCCTGCAGAGCGGCAACAGCCAGGAGAGCGTGACCGAGCAGGACAGCA |
| | | AGGACAGCACCTACAGCCTGAGCAGCACCCTGACCCTGAGCAAGGCCGACTA |
| | | CGAGAAGCACAAGGTGTACGCCTGCGAGGTGACCCACCAGGGCCTGAGCAGC |
| | | CCCGTGACCAAGAGCTTCAACAGGGGCGAGTGC |
| AL-001 | Heavy/ | CAGGTGCAGCTGGTGCAGAGCGGCGCCGAGGTGAAGAAGCCCGGCGCCAGCG |
| | SEQ ID | TGAAGGTGAGCTGCAAGGCCAGCGGCTACACCTTCACCAAGTACTACATGAG |
| | NO: 75 | CTGGGTGAGGCAGGCCCCCGGCCAGGGCCTGGAGTGGATGGGCATCATCAAC |
| | | CCCATCGGCGGCAGCACCAGCTACGCCCAGAAGTTCCAGGGCAGGGTGACCA |
| | | TGACCAGGGACACCAGCACCAGCACCGTGTACATGGAGCTGAGCAGCCTGAG |
| | | GAGCGAGGACACCGCCGTGTACTACTGCGCCAGGGACCCCAGCGGCATCGCC |
| | | CTGGCCGGCCCCGCCAGCAGGGGCTACCAGGGCATGGACGTGTGGGGCCAGG |
| | | GCACCACCGTGACCGTGAGCAGCGCCAGCACCAAGGGCCCCAGCGTGTTCCC |
| | | CCTGGCCCCCAGCAGCAAGAGCACCAGCGGCGGCACCGCCGCCCTGGGCTGC |
| | | CTGGTGAAGGACTACTTCCCCGAGCCCGTGACCGTGAGCTGGAACAGCGGCG |
| | | CCCTGACCAGCGGCGTGCACACCTTCCCCGCCGTGCTGCAGAGCAGCGGCCT |
| | | GTACAGCCTGAGCAGCGTGGTGACCGTGCCCAGCAGCAGCCTGGGCACCCAG |
| | | ACCTACATCTGCAACGTGAACCACAAGCCCAGCAACACCAAGGTGGACAAGA |
| | | GGGTGGAGCCCAAGAGCTGCGAC +/− AAGACCCACACC (or |
| | | AAGACCCACCTG) +/− TGCCCCCCCTGCCCCGCC +/− |
| | | CCCGAGCTGCTGGGCGGCCCCAGCGTGTTCCTG |
| AL-001 | Light/ | GAGATCGTGATGACCCAGAGCCCCGCCACCCTGAGCGTGAGCCCCGGCGAGA |
| | SEQ ID | GGGCCACCCTGAGCTGCAGGGCCAGCCAGAGCGTGAGCAGCAACCTGGCCTG |
| | NO: 76 | GTACCAGCAGAAGCCCGGCCAGGCCCCCAGGCTGCTGATCTACGGCGCCAGC |
| | | ACCAGGGCCACCGGCATCCCCGCCAGGTTCAGCGGCAGCGGCAGCGGCACCG |
| | | AGTTCACCCTGACCATCAGCAGCCTGCAGAGCGAGGACTTCGCCGTGTACTA |
| | | CTGCCAGCAGGCCAGGCTGGGCCCCTGGACCTTCGGCGGCGGCACCAAGGTG |
| | | GAGATCAAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| ABBV-8E12 | Heavy/ | GAGGTGAAGGTGGTGGAGAGCGGCGGCGGCCTGGTGCAGCCCGGCGGCAGCA |
| | SEQ ID | TGAAGCTGAGCTGCGTGGTGAGCGGCTTCACCTTCAGCAACTACTGGGTGAA |
| | NO: 77 | CTGGGTGAGGCAGGCCCCCGGCAAGGGCCTGGAGTGGGTGGCCCAGATCAGG |
| | | CTGAAGAGCGACAACTACGCCACCCACTACGAGGAGAGCGTGAAGGGCAGGT |
| | | TCACCATCAGCAGGGACGACAGCAAGAGCAGCGTGTACCTGCAGATGAACAA |
| | | CCTGAGGGCCGAGGACAGCGGCATCTACTACTGCACCAACTGGGAGGACTAC |
| | | TGGGGCCAGGGCACCACCGTGACCGTGAGCAGCGCCAGCACCAAGGGCCCCA |
| | | GCGTGTTCCCCCTGGCCCCCTGCAGCAGGAGCACCAGCGAGAGCACCGCCGC |
| | | CCTGGGCTGCCTGGTGAAGGACTACTTCCCCGAGCCCGTGACCGTGAGCTGG |
| | | AACAGCGGCGCCCTGACCAGCGGCGTGCACACCTTCCCCGCCGTGCTGCAGA |
| | | GCAGCGGCCTGTACAGCCTGAGCAGCGTGGTGACCGTGCCCAGCAGCAGCCT |
| | | GGGCACCAAGACCTACACCTGCAACGTGGACCACAAGCCCAGCAACACCAAG |
| | | GTGGACAAGAGGGTGGAGAGCAAGTAC +/− GGCCCCCCCTGCCCCCCCTG |
| | | CCCCGCC (GGCCCCCCCTGCCCCAGCTGCCCCGCC) +/− |
| | | CCCGAGTTCCTGGGCGGCCCCAGCGTGTTCCTG |
| ABBV-8E12 | Light/ | GACATCGTGCTGACCCAGAGCCCCGACAGCCTGGCCGTGAGCCTGGGCGAGA |
| | SEQ ID | GGGCCACCATCAGCTGCAGGGCCAGCCAGAGCGTGAGCACCAGCAGGTACAG |
| | NO: 78 | CTACATCCACTGGTACCAGCAGAAGCCCGGCCAGTTCCCCAAGCTGCTGATC |
| | | AAGTACGCCAGCAACCTGGAGAGCGGCGTGCCCAGCAGGTTCAGCGGCAGCG |
| | | GCAGCGGCACCGACTTCACCCTGAACATCCACCCCCTGGAGCCCGAGGACTT |
| | | CGCCACCTACTACTGCCACCACAGCTGGGAGATCCCCCTGACCTTCGGCCAG |
| | | GGCACCAAGCTGGAGATCAAGAGGACCGTGGCCGCCCCCAGCGTGTTCATCT |
| | | TCCCCCCCAGCGACGAGCAGCTGAAGAGCGGCACCGCCAGCGTGGTGTGCCT |
| | | GCTGAACAACTTCTACCCCAGGGAGGCCAAGGTGCAGTGGAAGGTGGACAAC |
| | | GCCCTGCAGAGCGGCAACAGCCAGGAGAGCGTGACCGAGCAGGACAGCAAGG |
| | | ACAGCACCTACAGCCTGAGCAGCACCCTGACCCTGAGCAAGGCCGACTACGA |
| | | GAAGCACAAGGTGTACGCCTGCGAGGTGACCCACCAGGGCCTGAGCAGCCCC |
| | | GTGACCAAGAGCTTCAACAGGGGCGAGTGC |
| UCB-0107 | Heavy/ | GAGGTGCAGCTGCAGGAGAGCGGCCCCGGCCTGGTGAAGCCCAGCGAGACCC |
| | SEQ ID | TGAGCCTGACCTGCACCGTGAGCGGCTTCAGCCTGACCAGCAACGACATCGC |
| | NO: 79 | CTGGATCAGGCAGCCCCCCGGCAAGGGCCTGGAGTGGATGGGCACCATCTGG |
| | | ACCGACGGCAGCACCAACTACAAC(GCC/ACC)GCCGTGCAGAGCAGGGTGA |
| | | CCATCAGCGTGGACACCAGCAAGAACCAGTTCAGCCTGAAGCTGAGCAGCGT |
| | | GACCGCCGCCGACACCGCCGTGTACTACTGCGCCAGGCACAGGCTGTACTAC |
| | | GGCGCCTTCGACTACTGGGGCCAGGGCACCATGGTGACCGTGAGCAGCGCCA |
| | | GCACCAAGGGCCCCAGCGTGTTCCCCCTGGCCCCCTGCAGCAGGAGCACCAG |
| | | CGAGAGCACCGCCGCCCTGGGCTGCCTGGTGAAGGACTACTTCCCCGAGCCC |
| | | GTGACCGTGAGCTGGAACAGCGGCGCCCTGACCAGCGGCGTGCACACCTTCC |
| | | CCGCCGTGCTGCAGAGCAGCGGCCTGTACAGCCTGAGCAGCGTGGTGACCGT |
| | | GCCCAGCAGCAGCCTGGGCACCAAGACCTACACCTGCAACGTGGACCACAAG |
| | | CCCAGCAACACCAAGGTGGACAAGAGGGTGGAGAGCAAGTAC +/− GGCCCC |
| | | CCCTGCCCCCCCTGCCCCGCC (GGCCCCCCCTGCCCCAGCTGCCCCGCC) |
| | | +/− CCCGAGTTCCTGGGCGGCCCCAGCGTGTTCCTG |
| UCB-0107 | Light/ | GACATCGTGATGACCCAGACCCCCCTGAGCCTGAGCGTGACCCCCGGCCAGC |
| | SEQ ID | CCGCCAGCATCAGCTGCAGGAGCAGCCAGAGCCTGGAGTACAGCGACGGCTA |
| | NO: 80 | CACCTACCTGGAGTGGTACCTGCAGAAGCCCGGCCAGAGCCCCCAGCTGCTG |
| | | ATCTACGAGGTGAGCAACAGGTTCAGCGGCGTGCCCGACAGGTTCAGCGGCA |
| | | GCGGCAGCGGCACCGACTTCACCCTGAAGATCAGCAGGGTGGAGGCCGAGGA |
| | | CGTGGGCGTGTACTACTGCTTCCAGGCCACCCACAACCCCTACACCTTCGGC |
| | | CAGGGCACCAAGCTGGAGATCAAGAGGACCGTGGCCGCCCCCAGCGTGTTCA |
| | | TCTTCCCCCCCAGCGACGAGCAGCTGAAGAGCGGCACCGCCAGCGTGGTGTG |
| | | CCTGCTGAACAACTTCTACCCCAGGGAGGCCAAGGTGCAGTGGAAGGTGGAC |
| | | AACGCCCTGCAGAGCGGCAACAGCCAGGAGAGCGTGACCGAGCAGGACAGCA |
| | | AGGACAGCACCTACAGCCTGAGCAGCACCCTGACCCTGAGCAAGGCCGACTA |
| | | CGAGAAGCACAAGGTGTACGCCTGCGAGGTGACCCACCAGGGCCTGAGCAGC |
| | | CCCGTGACCAAGAGCTTCAACAGGGGCGAGTGC |
| NI-105 | Heavy/ | CAGGTGCAGCTGGTGGAGAGCGGCGGCGGCGTGGTGCAGCCCGGCAGGAGCC |
| | SEQ ID | TGAGGGTGAGCTGCGCCGCCAGCGGCTTCACCTTCAGCAGCTACGACATGCA |
| | NO: 81 | CTGGGTGAGGCAGGCCCCCGGCAAGGGCCTGGAGTGGGTGGCCGTGATCTGG |
| | | TTCGACGGCAGCAACGAGTTCTACGCCGACAGCGTGAAGGGCAGGTTCACCA |
| | | TCAGCAGGGACAACAGCAAGAACACCCTGTTCCAGATGAACAGCCTGAGGGC |
| | | CGAGGACACCGCCGTGTACTACTGCGCCAGGGACCTGGGCGCCAGCGTGACC |
| | | ACCAGCAACGCCGAGAACTTCCACCACTGGGGCCAGGGCACCCTGGTGACCG |
| | | TGAGCAGCGCCAGCACCAAGGGCCCCAGCGTGTTCCCCCTGGCCCCCAGCAG |
| | | CAAGAGCACCAGCGGCGGCACCGCCGCCCTGGGCTGCCTGGTGAAGGACTAC |
| | | TTCCCCGAGCCCGTGACCGTGAGCTGGAACAGCGGCGCCCTGACCAGCGGCG |
| | | TGCACACCTTCCCCGCCGTGCTGCAGAGCAGCGGCCTGTACAGCCTGAGCAG |
| | | CGTGGTGACCGTGCCCAGCAGCAGCCTGGGCACCCAGACCTACATCTGCAAC |
| | | GTGAACCACAAGCCCAGCAACACCAAGGTGGACAAGAAGGTGGAGCCCAAGA |
| | | GCTGCGAC +/− AAGACCCACACC (or AAGACCCACCTG) +/− |
| | | TGCCCCCCCTGCCCCGCC +/− CCCGAGCTGGCCGGCGCCCCCAGC |
| | | GTGTTCCTG |
| NI-105 | Light/ | AGCTACGAGCTGACCCAGCCCCCCAGCGTGAGCGTGAGCCCCGGCCAGACCG |
| | SEQ ID | CCAGGATCACCTGCAGCGGCGACGCCCTGCCCAAGAGGTACGTGTACTGGTA |
| | NO: 82 | CCAGCAGAAGAGCGGCCAGGCCCCCGTGCTGGTGATCTACGAGGACAGCAAG |
| | | AGGCCCAGCGGCATCCCCGAGACCTTCAGCGGCAGCAGCAGCGGCACCATGG |
| | | CCACCCTGACCATCAGCGGCGCCCAGGTGGAGGACGAGGCCGACTACTACTG |
| | | CTACAGCACCGACAGCAACGGCCACCACTGGGTGTTCGGCGGCGGCACCAAG |
| | | CTGACCGTGCTGGGCCAGCCCAAGGCCGCCCCCAGCGTGACCCTGTTCCCCC |
| | | CCAGCAGCGAGGAGCTGCAGGCCAACAAGGCCACCCTGGTGTGCCTGATCAG |
| | | CGACTTCTACCCCGGCGCCGTGACCGTGGCCTGGAAGGCCGACAGCAGCCCC |
| | | GTGAAGGCCGGCGTGGAGACCACCACCCCCAGCAAGCAGAGCAACAACAAGT |
| | | ACGCCGCCAGCAGCTACCTGAGCCTGACCCCCGAGCAGTGGAAGAGCCACAG |
| | | GAGCTACAGCTGCCAGGTGACCCACGAGGGCAGCACCGTGGAGAAGACCGTG |
| | | GCCCCCACCTGCAGC |
| VX15/2503 | Heavy/ | CAGGTGCAGCTGGTGCAGAGCGGCGCCGAGGTGAAGAAGCCCGGCAGCAGCG |
| | SEQ ID | TGAAGGTGAGCTGCAAGGCCAGCGGCTACAGCTTCAGCGACTACTACATGCA |
| | NO: 83 | CTGGGTGAGGCAGGCCCCCGGCCAGGGCCTGGAGTGGATGGGCCAGATCAAC |
| | | CCCACCACCGGCGGCGCCAGCTACAACCAGAAGTTCAAGGGCAAGGCCACCA |
| | | TCACCGTGGACAAGAGCACCAGCACCGCCTACATGGAGCTGAGCAGCCTGAG |
| | | GAGCGAGGACACCGCCGTGTACTACTGCGCCAGGTACTACTACGGCAGGCAC |
| | | TTCGACGTGTGGGGCCAGGGCACCACCGTGACCGTGAGCAGCGCCAGCACCA |
| | | AGGGCCCCAGCGTGTTCCCCCTGGCCCCCTGCAGCAGGAGCACCAGCGAGAG |
| | | CACCGCCGCCCTGGGCTGCCTGGTGAAGGACTACTTCCCCGAGCCCGTGACC |
| | | GTGAGCTGGAACAGCGGCGCCCTGACCAGCGGCGTGCACACCTTCCCCGCCG |
| | | TGACCGTGAGCAGCGTGCTGCAGAGCAGCGGCCTGTACAGCCTGAGCAGCGT |
| | | GGTGACCGTGCCCAGCAGCAGCCTGGGCACCAAGACCTACACCTGCAACGTG |
| | | GACCACAAGCCCAGCAACACCAAGGTGGACAAGAGGGTGGAGAGCAAGTAC |
| | | +/− GGCCCCCCCTGCCCCCCCTGCCCCGCC |
| | | (GGCCCCCCCTGCCCCAGCTGCCCCGCC) +/− CCCGAGTTCCTGGGCG |
| | | GCCCCAGCGTGTTCCTG |
| VX15/2503 | Light/ | GACATCGTGATGACCCAGAGCCCCGACAGCCTGGCCGTGAGCCTGGGCGAGA |
| | SEQ ID | GGGCCACCATCAACTGCAAGGCCAGCCAGAGCGTGGACTACGACGGCGACAG |
| | NO: 84 | CTACATGAACTGGTACCAGCAGAAGCCCGGCCAGCCCCCCAAGCTGCTGATC |
| | | TACGCCGCCAGCAACCTGGAGAGCGGCGTGCCCGACAGGTTCAGCGGCAGCG |
| | | GCAGCGGCACCGACTTCACCCTGACCATCAGCAGCCTGCAGGCCGAGGACGT |
| | | GGCCGTGTACTACTGCCAGCAGAGCAACGAGGACCCCTACACCTTCGGCCAG |
| | | GGCACCAAGCTGGAGATCAAGTTCATCTTCCCCCCCAGCGACGAGCAGCTGA |
| | | AGAGCGGCACCGCCAGCGTGGTGTGCCTGCTGAACAACTTCTACCCCAGGGA |
| | | GGCCAAGGTGCAGTGGAAGGTGGACAACGCCCTGCAGAGCGGCAACAGCCAG |
| | | GAGAGCGTGACCGAGCAGGACAGCAAGGACAGCACCTACAGCCTGAGCAGCA |
| | | CCCTGACCCTGAGCAAGGCCGACTACGAGAAGCACAAGGTGTACGCCTGCGA |
| | | GGTGACCCACCAGGGCCTGAGCAGCCCCGTGACCAAGAGCTTCAACAGGGGC |
| | | GAGTGC |
| Prasinezumab | Heavy/ | GAGGTGCAGCTGGTGGAGAGCGGCGGCGGCCTGGTGCAGCCCGGCGGCAGCC |
| | SEQ ID | TGAGGCTGAGCTGCGCCGCCAGCGGCTTCACCTTCAGCAACTACGGCATGAG |
| | NO: 85 | CTGGGTGAGGCAGGCCCCCGGCAAGGGCCTGGAGTGGGTGGCCAGCATCAGC |
| | | AGCGGCGGCGGCAGCACCTACTACCCCGACAACGTGAAGGGCAGGTTCACCA |
| | | TCAGCAGGGACGACGCCAAGAACAGCCTGTACCTGCAGATGAACAGCCTGAG |
| | | GGCCGAGGACACCGCCGTGTACTACTGCGCCAGGGGCGGCGCCGGCATCGAC |
| | | TACTGGGGCCAGGGCACCCTGGTGACCGTGAGCAGCGCCAGCACCAAGGGCC |
| | | CCAGCGTGTTCCCCCTGGCCCCCAGCAGCAAGAGCACCAGCGGCGGCACCGC |
| | | CGCCCTGGGCTGCCTGGTGAAGGACTACTTCCCCGAGCCCGTGACCGTGAGC |
| | | TGGAACAGCGGCGCCCTGACCAGCGGCGTGCACACCTTCCCCGCCGTGCTGC |
| | | AGAGCAGCGGCCTGTACAGCCTGAGCAGCGTGGTGACCGTGCCCAGCAGCAG |
| | | CCTGGGCACCCAGACCTACATCTGCAACGTGAACCACAAGCCCAGCAACACC |
| | | AAGGTGGACAAGAGGGTGGAGCCCAAGAGCTGCGAC +/− AAGACCCACACC |
| | | (or AAGACCCACCTG) +/− TGCCCCCCCTGCCCCGCC +/− |
| | | CCCGAGCTGCTGGGCGGCCCCAGCGTGTTCCTG |
| Prasinezumab | Light/ | GACATCCAGATGACCCAGAGCCCCAGCAGCCTGAGCGCCAGCGTGGGCGACA |
| | SEQ ID | GGGTGACCATCACCTGCAAGAGCATCCAGACCCTGCTGTACAGCAGCAACCA |
| | NO: 86 | GAAGAACTACCTGGCCTGGTTCCAGCAGAAGCCCGGCAAGGCCCCCAAGCTG |
| | | CTGATCTACTGGGCCAGCATCAGGAAGAGCGGCGTGCCCAGCAGGTTCAGCG |
| | | GCAGCGGCAGCGGCACCGACTTCACCCTGACCATCAGCAGCCTGCAGCCCGA |
| | | GGACCTGGCCACCTACTACTGCCAGCAGTACTACAGCTACCCCCTGACCTTC |
| | | GGCGGCGGCACCAAGCTGGAGATCAAGAGGACCGTGGCCGCCCCCAGCGTGT |
| | | TCATCTTCCCCCCCAGCGACGAGCAGCTGAAGAGCGGCACCGCCAGCGTGGT |
| | | GTGCCTGCTGAACAACTTCTACCCCAGGGAGGCCAAGGTGCAGTGGAAGGTG |
| | | GACAACGCCCTGCAGAGCGGCAACAGCCAGGAGAGCGTGACCGAGCAGGACA |
| | | GCAAGGACAGCACCTACAGCCTGAGCAGCACCCTGACCCTGAGCAAGGCCGA |
| | | CTACGAGAAGCACAAGGTGTACGCCTGCGAGGTGACCCACCAGGGCCTGAGC |
| | | AGCCCCGTGACCAAGAGCTTCAACAGGGGCGAGTGC |
| NI-202 | Heavy/ | GAGGTGCAGCTGGTGGAGAGCGGCGCCGAGGTGAAGAAGCCCGGCGCCAGCG |
| | SEQ ID | TGAAGGTGAGCTGCAAGGCCAGCGGCTACACCTTCACCAACTACGCCATGCA |
| | NO: 87 | CTGGGTGAGGCAGGCCCCCGGCCAGAGGCTGGAGTGGATGGGCTGGATCAAC |
| | | GCCGGCAACGGCAAGAGGAAGTACAGCCAGAAGTTCCAGGACAGGGTGACCA |
| | | TCAACAGGGACACCAGCGCCAGCACCATCTACATGGAGCTGAGCAGCCTGGG |
| | | CAGCGAGGACACCGCCGTGTACTACTGCGCCAGGGAGGAGGACCACGCCGGC |
| | | AGCGGCAGCTACCTGAGCATGGACGTGTGGGGCCAGGGCACCCTGGTGACCG |
| | | TGAGCAGCGCCAGCACCAAGGGCCCCAGCGTGTTCCCCCTGGCCCCCAGCAG |
| | | CAAGAGCACCAGCGGCGGCACCGCCGCCCTGGGCTGCCTGGTGAAGGACTAC |
| | | TTCCCCGAGCCCGTGACCGTGAGCTGGAACAGCGGCGCCCTGACCAGCGGCG |
| | | TGCACACCTTCCCCGCCGTGCTGCAGAGCAGCGGCCTGTACAGCCTGAGCAG |
| | | CGTGGTGACCGTGCCCAGCAGCAGCCTGGGCACCCAGACCTACATCTGCAAC |
| | | GTGAACCACAAGCCCAGCAACACCAAGGTGGACAAGAGGGTGGAGCCCAAGA |
| | | GCTGCGAC +/− AAGACCCACACC (or AAGACCCACCTG) +/− |
| | | TGCCCCCCCTGCCCCGCC +/− CCCGAGCTGCTGGGCGGCCCCAGCGTGTT |
| | | CCTG |
| NI-202 | Light/ | GACATCCAGATGACCCAGAGCCCCAGCAGCCTGAGCGCCAGCGTGGGCGACA |
| | SEQ ID | GGGTGACCATCACCTGCAAGAGCATCCAGACCCTGCTGTACAGCAGCAACCA |
| | NO: 88 | GAAGAACTACCTGGCCTGGTTCCAGCAGAAGCCCGGCAAGGCCCCCAAGCTG |
| | | CTGATCTACTGGGCCAGCATCAGGAAGAGCGGCGTGCCCAGCAGGTTCAGCG |
| | | GCAGCGGCAGCGGCACCGACTTCACCCTGACCATCAGCAGCCTGCAGCCCGA |
| | | GGACCTGGCCACCTACTACTGCCAGCAGTACTACAGCTACCCCCTGACCTTC |
| | | GGCGGCGGCACCAAGCTGGAGATCAAGAGGACCGTGGCCGCCCCCAGCGTGT |
| | | TCATCTTCCCCCCCAGCGACGAGCAGCTGAAGAGCGGCACCGCCAGCGTGGT |
| | | GTGCCTGCTGAACAACTTCTACCCCAGGGAGGCCAAGGTGCAGTGGAAGGTG |
| | | GACAACGCCCTGCAGAGCGGCAACAGCCAGGAGAGCGTGACCGAGCAGGACA |
| | | GCAAGGACAGCACCTACAGCCTGAGCAGCACCCTGACCCTGAGCAAGGCCGA |
| | | CTACGAGAAGCACAAGGTGTACGCCTGCGAGGTGACCCACCAGGGCCTGAGC |
| | | AGCCCCGTGACCAAGAGCTTCAACAGGGGCGAGTGC |
| MEDI- | Heavy/ | GAGGTGCAGCTGCTGGAGAGCGGCGGCGGCCTGGTGCAGCCCGGCGGCAGCC |
| 1341/TAK 341 | SEQ ID | TGAGGCTGAGCTGCGCCGCCAGCGGCTTCACCTTCAGCAGCTACGCCATGAG |
| | NO: 89 | CTGGGTGAGGCAGGCCCCCGGCAAGGGCCTGGAGTGGGTGAGCAGCATCAGC |
| | | CACCTGGGCGGCAGCACCTACTACGCCGACAGCGTGAAGGGCAGGTTCACCA |
| | | TCAGCAGGGACAACAGCAAGAACACCCTGTACCTGCAGATGAACAGCCTGAG |
| | | GGCCGAGGACACCGCCGTGTACTACTGCGCCGGCGGCGCCAACCACGGCAAG |
| | | TACTACTACGGCATGGACAAGTGGGGCCAGGGCACCACCGTGACCGTGAGCA |
| | | GCGCCAGCACCAAGGGCCCCAGCGTGTTCCCCCTGGCCCCCAGCAGCAAGAG |
| | | CACCAGCGGCGGCACCGCCGCCCTGGGCTGCCTGGTGAAGGACTACTTCCCC |
| | | GAGCCCGTGACCGTGAGCTGGAACAGCGGCGCCCTGACCAGCGGCGTGCACA |
| | | CCTTCCCCGCCGTGCTGCAGAGCAGCGGCCTGTACAGCCTGAGCAGCGTGGT |
| | | GACCGTGCCCAGCAGCAGCCTGGGCACCCAGACCTACATCTGCAACGTGAAC |
| | | CACAAGCCCAGCAACACCAAGGTGGACAAGAGGGTGGAGCCCAAGAGCTGCG |
| | | AC +/− AAGACCCACACC (or AAGACCCACCTG) +/− |
| | | TGCCCCCCCTGCCCCGCC +/− CCCGAGTTCGAGGGCGGCCCCAGCG |
| | | TGTTCCTG |
| MEDI- | Light/ | CAGGCCGTGCTGACCCAGCCCGCCAGCCTGAGCGCCAGCCCCGGCGCCAGCG |
| 1341/TAK 341 | SEQ ID | CCAGCCTGACCTGCACCCTGAGGAGCGGCGCCCCCCTGCCCAAGTACAGGAT |
| | NO: 90 | CTACTGGTACCAGCAGAAGCCCGGCAGCCCCCCCCAGTACCTGCTGAGGTAC |
| | | AAGAGCGACGCCGACAAGCACCAGGGCAGCGGCGTGCCCAGCAGGTTCAGCG |
| | | GCAGCAAGGACGCCAGCGCCAACGCCGGCATCCTGCTGATCAGCGGCCTGCA |
| | | GAGCGAGGACGAGGCCGACTACTACTGCATGGTGTGGGACCACGGCGTGTGG |
| | | TACTTCGGCGGCGGCACCAAGCTGACCGTGCTGGGCCAGCCCAAGGCCGCCC |
| | | CCAGCGTGACCCTGTTCCCCCCCAGCAGCGAGGAGCTGCAGGCCAACAAGGC |
| | | CACCCTGGTGTGCCTGATCAGCGACTTCTACCCCGGCGCCGTGACCGTGGCC |
| | | TGGAAGGCCGACAGCAGCCCCGTGAAGGCCGGCGTGGAGACCACCACCCCCA |
| | | GCAAGCAGAGCAACAACAAGTACGCCGCCAGCAGCTACCTGAGCCTGACCCC |
| | | CGAGCAGTGGAAGAGCCACAGGAGCTACAGCTGCCAGGTGACCCACGAGGGC |
| | | AGCACCGTGGAGAAGACCGTGGCCCCCACCGAGTGCAGC |
| NI-204.10D12 | Heavy/ | GAGGTGCAGCTGGTGGAGAGCGGCGGCGACCTGGTGAGGCCCGGCGGCAGCC |
| | SEQ ID | TGAGGCTGAGCTGCGTGGCCAGCGGCTTCACCTTCAGCAACTACTGGATGCA |
| | NO: 91 | CTGGGTGAGGCAGGCCCCCGGCCAGAGGCCCGTGTGGGTGAGCAGGACCAAC |
| | | ACCGACGGCAGGAACACCGCCTACGCCGACTACGCCAAGGGCAGGTTCACCA |
| | | TCAGCAGGGACAACGCCAAGAGCACCCTGTACCTGCAGCTGAACAGCCTGAG |
| | | GGCCGAGGACACCGCCGTGTACTTCTGCGCCAGGCTGAGGAGGAACGTGGCC |
| | | GACCAGATCACCCACAACTACTACATGGACGTGTGGGGCAAGGGCACCCTGG |
| | | TGACCGTGAGCAGCGCCAGCACCAAGGGCCCCAGCGTGTTCCCCCTGGCCCC |
| | | CAGCAGCAAGAGCACCAGCGGCGGCACCGCCGCCCTGGGCTGCCTGGTGAAG |
| | | GACTACTTCCCCGAGCCCGTGACCGTGAGCTGGAACAGCGGCGCCCTGACCA |
| | | GCGGCGTGCACACCTTCCCCGCCGTGCTGCAGAGCAGCGGCCTGTACAGCCT |
| | | GAGCAGCGTGGTGACCGTGCCCAGCAGCAGCCTGGGCACCCAGACCTACATC |
| | | TGCAACGTGAACCACAAGCCCAGCAACACCAAGGTGGACAAGAAGGTGGAGC |
| | | CCAAGAGCTGCGAC +/− AAGACCCACACC (or AAGACCCACCTG) +/− |
| | | TGCCCCCCCTGCCCCGCC +/− CCCGAGGCCGCCGGCGGCCCCAGC |
| | | GTGTTCCTG |
| NI-204.10D12 | Light/ | GACATCCAGATGACCCAGAGCCCCAGCAGCCTGAGCGCCAGCGTGGGCGACA |
| | SEQ ID | GGGTGACCGCCTGCAGGGCCAGCCAGAGCGTGGGCACCTACCTGAACTGGTA |
| | NO: 92 | CCAGCAGAAGAGGGGCAAGGCCCCCAAGCTGCTGATCTTCGCCGCCAGCAGC |
| | | CTGCAGAGCGGCGTGCCCAGCAGGTTCAGCGGCAGCGGCAGCGGCACCGACT |
| | | TCACCCTGACCATCAGCAGCCTGCAGCCCGAGGACTTCGCCACCTACTACTG |
| | | CCAGCAGAGCTACAGCAGCCCCCCCACCTTCGGCCAGGGCACCAAGGTGGAG |
| | | ATCAAGAGGACCGTGGCCGCCCCCAGCGTGTTCATCTTCCCCCCCAGCGACG |
| | | AGCAGCTGAAGAGCGGCACCGCCAGCGTGGTGTGCCTGCTGAACAACTTCTA |
| | | CCCCAGGGAGGCCAAGGTGCAGTGGAAGGTGGACAACGCCCTGCAGAGCGGC |
| | | AACAGCCAGGAGAGCGTGACCGAGCAGGACAGCAAGGACAGCACCTACAGCC |
| | | TGAGCAGCACCCTGACCCTGAGCAAGGCCGACTACGAGAAGCACAAGGTGTA |
| | | CGCCTGCGAGGTGACCCACCAGGGCCTGAGCAGCCCCGTGACCAAGAGCTTC |
| | | AACAGGGGCGAGTGC |
| NI-204.12G7 | Heavy/ | CAGGTGCAGCTGGTGCAGAGCGGCGCCGAGGTGAAGAAGCCCGGCGCCAGCG |
| | SEQ ID | TGACCCTGAGCTGCAAGGCCAGCGGCTACACCTTCACCGCCTACTACATCCA |
| | NO: 93 | CTGGGTGAGGCAGGCCAGGGAGCAGGGCCTGGAGTGGATGGGCGTGATCAAC |
| | | CCCAGCACCGGCACCACCTTCTACGCCCAGAACTTCCCCGACAGGGTGAGCG |
| | | TGACCAGGGACACCAGCACCAGCACCGTGTTCATGGAGCTGCACAACCTGAA |
| | | GAGCGAGGACACCGCCGTGTACTACTGCGCCAGGGCCATCAGCGAGCACGGC |
| | | AGCGGCAGCTACAGCCCCTACTACTGGGGCCAGGGCACCCTGGTGACCGTGA |
| | | GCAGCGCCAGCACCAAGGGCCCCAGCGTGTTCCCCCTGGCCCCCAGCAGCAA |
| | | GAGCACCAGCGGCGGCACCGCCGCCCTGGGCTGCCTGGTGAAGGACTACTTC |
| | | CCCGAGCCCGTGACCGTGAGCTGGAACAGCGGCGCCCTGACCAGCGGCGTGC |
| | | ACACCTTCCCCGCCGTGCTGCAGAGCAGCGGCCTGTACAGCCTGAGCAGCGT |
| | | GGTGACCGTGCCCAGCAGCAGCCTGGGCACCCAGACCTACATCTGCAACGTG |
| | | AACCACAAGCCCAGCAACACCAAGGTGGACAAGAAGGTGGAGCCCAAGAGCT |
| | | GCGAC +/− AAGACCCACACC (or AAGACCCACCTG) +/− |
| | | TGCCCCCCCTGCCCCGCC +/− CCCGAGGCCGCCGGCGGCCCCAGCGT |
| | | GTTCCTG |
| NI-204.12G7 | Light/ | AGCTACGAGCTGACCCAGCCCCCCAGCGTGAGCGTGAGCCTGGGCCAGATGG |
| | SEQ ID | CCGCCATCACCTGCAGCGGCGAGGCCCTGCCCAAGAAGTACGGCTACTGGTA |
| | NO: 94 | CCAGCAGAAGCCCGGCCAGGTGCCCGTGCTGCTGATCTACAGGGACGTGGAG |
| | | AGGCCCAGCGGCGTGCCCGACAGGTTCAGCGGCAGCAGCAGCGGCACCATGG |
| | | TGACCCTGACCATCAGCGGCGTGCAGGCCGAGGACGAGGCCGACTACTACTG |
| | | CCTGAGCGCCGACAGCAGCGGCACCTGGGTGTTCGGCGGCGGCACCAAGCTG |
| | | ACCGTGCTG |
| Eptinezumab | Heavy/ | GAGGTGCAGCTGGTGGAGAGCGGCGGCGGCCTGGTGCAGCCCGGCGGCAGCC |
| | SEQ ID | TGAGGCTGAGCTGCGCCGTGAGCGGCATCGACCTGAGCGGCTACTACATGAA |
| | NO: 95 | CTGGGTGAGGCAGGCCCCCGGCAAGGGCCTGGAGTGGGTGGGCGTGATCGGC |
| | | ATCAACGGCGCCACCTACTACGCCAGCTGGGCCAAGGGCAGGTTCACCATCA |
| | | GCAGGGACAACAGCAAGACCACCGTGTACCTGCAGATGAACAGCCTGAGGGC |
| | | CGAGGACACCGCCGTGTACTTCTGCGCCAGGGGCGACATCTGGGGCCAGGGC |
| | | ACCCTGGTGACCGTGAGCAGCGCCAGCACCAAGGGCCCCAGCGTGTTCCCCC |
| | | TGGCCCCCAGCAGCAAGAGCACCAGCGGCGGCACCGCCGCCCTGGGCTGCCT |
| | | GGTGAAGGACTACTTCCCCGAGCCCGTGACCGTGAGCTGGAACAGCGGCGCC |
| | | CTGACCAGCGGCGTGCACACCTTCCCCGCCGTGCTGCAGAGCAGCGGCCTGT |
| | | ACAGCCTGAGCAGCGTGGTGACCGTGCCCAGCAGCAGCCTGGGCACCCAGAC |
| | | CTACATCTGCAACGTGAACCACAAGCCCAGCAACACCAAGGTGGACGCCAGG |
| | | GTGGAGCCCAAGAGCTGCGAC +/− AAGACCCACACC (or |
| | | AAGACCCACCTG) +/− TGCCCCCCCTGCCCCGCC +/− |
| | | CCCGAGCTGCTGGGCGGCCCCAGCGTGACCCTG |
| Eptinezumab | Light/ | CAGGTGCTGACCCAGAGCCCCAGCAGCCTGAGCGCCAGCGTGGGCGACAGGG |
| | SEQ ID | TGACCATCAACTGCCAGGCCAGCCAGAGCGTGTACCACAACACCTACCTGGC |
| | NO: 96 | CTGGTACCAGCAGAAGCCCGGCAAGGTGCCCAAGCAGCTGATCTACGACGCC |
| | | AGCACCCTGGCCAGCGGCGTGCCCAGCAGGTTCAGCGGCAGCGGCAGCGGCA |
| | | CCGACTTCACCCTGACCATCAGCAGCCTGCAGCCCGAGGACGTGGCCACCTA |
| | | CTACTGCCTGGGCAGCTACGACTGCACCAACGGCGACTGCTTCGTGTTCGGC |
| | | GGCGGCACCAAGGTGGAGATCAAGAGGACCGTGGCCGCCCCCAGCGTGTTCA |
| | | TCTTCCCCCCCAGCGACGAGCAGCTGAAGAGCGGCACCGCCAGCGTGGTGTG |
| | | CCTGCTGAACAACTTCTACCCCAGGGAGGCCAAGGTGCAGTGGAAGGTGGAC |
| | | AACGCCCTGCAGAGCGGCAACAGCCAGGAGAGCGTGACCGAGCAGGACAGCA |
| | | AGGACAGCACCTACAGCCTGAGCAGCACCCTGACCCTGAGCAAGGCCGACTA |
| | | CGAGAAGCACAAGGTGTACGCCTGCGAGGTGACCCACCAGGGCCTGAGCAGC |
| | | CCCGTGACCAAGAGCTTCAACAGGGGCGAGTGC |
| Fremanezumab | Heavy/ | GAGGTGCAGCTGGTGGAGAGCGGCGGCGGCCTGGTGCAGCCCGGCGGCAGCC |
| | SEQ ID | TGAGGCTGAGCTGCGCCGCCAGCGGCTTCACCTTCAGCAACTACTGGATCAG |
| | NO: 97 | CTGGGTGAGGCAGGCCCCCGGCAAGGGCCTGGAGTGGGTGGCCGAGATCAGG |
| | | AGCGAGAGCGACGCCAGCGCCACCCACTACGCCGAGGCCGTGAAGGGCAGGT |
| | | TCACCATCAGCAGGGACAACGCCAAGAACAGCCTGTACCTGCAGATGAACAG |
| | | CCTGAGGGCCGAGGACACCGCCGTGTACTACTGCCTGGCCTACTTCGACTAC |
| | | GGCCTGGCCATCCAGAACTACTGGGGCCAGGGCACCCTGGTGACCGTGAGCA |
| | | GCGCCAGCACCAAGGGCCCCAGCGTGTTCCCCCTGGCCCCCTGCAGCAGGAG |
| | | CACCAGCGAGAGCACCGCCGCCCTGGGCTGCCTGGTGAAGGACTACTTCCCC |
| | | GAGCCCGTGACCGTGAGCTGGAACAGCGGCGCCCTGACCAGCGGCGTGCACA |
| | | CCTTCCCCGCCGTGCTGCAGAGCAGCGGCCTGTACAGCCTGAGCAGCGTGGT |
| | | GACCGTGCCCAGCAGCAACTTCGGCACCCAGACCTACACCTGCAACGTGGAC |
| | | CACAAGCCCAGCAACACCAAGGTGGACAAGACCGTGGAGAGGAAGTGCTGCG |
| | | TGGAG +/− TGCCCCCCCTGCCCCGCC +/− CCCCCCGTGGCCGGC |
| Fremanezumab | Light/ | GAGATCGTGCTGACCCAGAGCCCCGCCACCCTGAGCCTGAGCCCCGGCGAGA |
| | SEQ ID | GGGCCACCCTGAGCTGCAAGGCCAGCAAGAGGGTGACCACCTACGTGAGCTG |
| | NO: 98 | GTACCAGCAGAAGCCCGGCCAGGCCCCCAGGCTGCTGATCTACGGCGCCAGC |
| | | AACAGGTACCTGGGCATCCCCGCCAGGTTCAGCGGCAGCGGCAGCGGCACCG |
| | | ACTTCACCCTGACCATCAGCAGCCTGGAGCCCGAGGACTTCGCCGTGTACTA |
| | | CTGCAGCCAGAGCTACAACTACCCCTACACCTTCGGCCAGGGCACCAAGCTG |
| | | GAGATCAAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| Galcanezumab | Heavy/ | CAGGTGCAGCTGGTGCAGAGCGGCGCCGAGGTGAAGAAGCCCGGCAGCAGCG |
| | SEQ ID | TGAAGGTGAGCTGCAAGGCCAGCGGCTACACCTTCGGCAACTACTGGATGCA |
| | NO: 99 | GTGGGTGAGGCAGGCCCCCGGCCAGGGCCTGGAGTGGATGGGCGCCATCTAC |
| | | GAGGGCACCGGCAAGACCGTGTACATCCAGAAGTTCGCCGACAGGGTGACCA |
| | | TCACCGCCGACAAGAGCACCAGCACCGCCTACATGGAGCTGAGCAGCCTGAG |
| | | GAGCGAGGACACCGCCGTGTACTACTGCGCCAGGCTGAGCGACTACGTGAGC |
| | | GGCTTCGGCTACTGGGGCCAGGGCACCACCGTGACCGTGAGCAGCGCCAGCA |
| | | CCAAGGGCCCCAGCGTGTTCCCCCTGGCCCCCTGCAGCAGGAGCACCAGCGA |
| | | GAGCACCGCCGCCCTGGGCTGCCTGGTGAAGGACTACTTCCCCGAGCCCGTG |
| | | ACCGTGAGCTGGAACAGCGGCGCCCTGACCAGCGGCGTGCACACCTTCCCCG |
| | | CCGTGCTGCAGAGCAGCGGCCTGTACAGCCTGAGCAGCGTGGTGACCGTGCC |
| | | CAGCAGCAGCCTGGGCACCAAGACCTACACCTGCAACGTGGACCACAAGCCC |
| | | AGCAACACCAAGGTGGACAAGAGGGTGGAGAGCAAGTAC +/− GGCCCCCCC |
| | | TGCCCCCCCTGCCCCGCC (GGCCCCCCCTGCCCCAGCTGCCCCGCC) +/− |
| | | CCCGAGGCCGCCGGCGGCCCCAGCGTGTTCCTG |
| Galcanezumab | Light/ | GACATCCAGATGACCCAGAGCCCCAGCAGCCTGAGCGCCAGCGTGGGCGACA |
| | SEQ ID | GGGTGACCATCACCTGCAGGGCCAGCAAGGACATCAGCAAGTACCTGAACTG |
| | NO: 100 | GTACCAGCAGAAGCCCGGCAAGGCCCCCAAGCTGCTGATCTACTACACCAGC |
| | | GGCTACCACAGCGGCGTGCCCAGCAGGTTCAGCGGCAGCGGCAGCGGCACCG |
| | | ACTTCACCCTGACCATCAGCAGCCTGCAGCCCGAGGACTTCGCCACCTACTA |
| | | CTGCCAGCAGGGCGACGCCCTGCCCCCCACCTTCGGCGGCGGCACCAAGGTG |
| | | GAGATCAAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| Sevacizumab | Heavy/ | GAGGTGCAGCTGGTGGAGAGCGGCGGCGGCCTGGTGAAGCCCGGCGGCAGCC |
| | SEQ ID | TGAGGCTGAGCTGCGCCGCCAGCGGCTTCAGCTTCAGCAACAACGACGTGAT |
| | NO: 101 | GTGCTGGGTGAGGCAGGCCCCCGGCAAGGGCCTGGAGTGGATCGGCTGCATC |
| | | ATGACCACCGACGTGGTGACCGAGTACGCCAACTGGGCCAAGAGCAGGTTCA |
| | | CCGTGAGCAGGGACAGCGCCAAGAACAGCGTGTACCTGCAGATGAACAGCCT |
| | | GAGGGCCGAGGACACCGCCGTGTACTTCTGCGCCAGGGACAGCGTGGGCAGC |
| | | CCCCTGATGAGCTTCGACCTGTGGGGCCCCGGCACCCTGGTGACCGTGAGCA |
| | | GCGCCAGCACCAAGGGCCCCAGCGTGTTCCCCCTGGCCCCCAGCAGCAAGAG |
| | | CACCAGCGGCGGCACCGCCGCCCTGGGCTGCCTGGTGAAGGACTACTTCCCC |
| | | GAGCCCGTGACCGTGAGCTGGAACAGCGGCGCCCTGACCAGCGGCGTGCACA |
| | | CCTTCCCCGCCGTGCTGCAGAGCAGCGGCCTGTACAGCCTGAGCAGCGTGGT |
| | | GACCGTGCCCAGCAGCAGCCTGGGCACCCAGACCTACATCTGCAACGTGAAC |
| | | CACAAGCCCAGCAACACCAAGGTGGACAAGAAGGTGGAGCCCAAGAGCTGCG |
| | | AC +/− AAGACCCACACC (or AAGACCCACCTG) +/− |
| | | TGCCCCCCCTGCCCCGCC +/− CCCGAGCTGCTGGGCGGCCCCAGCGTGTT |
| | | CCTG |
| Sevacizumab | Light/ | GACATCCAGATGACCCAGAGCCCCAGCAGCCTGAGCGCCAGCGTGGGCGACA |
| | SEQ ID | GGGTGACCATCAACTGCCAGGCCAGCCAGAGCATCTACAACAACAACGAGCT |
| | NO: 102 | GAGCTGGTACCAGCAGAAGCCCGGCAAGCCCCCCAAGCTGCTGATCTACAGG |
| | | GCCAGCACCCTGGCCAGCGGCGTGCCCAGCAGGTTCAGCGGCAGCGGCAGCG |
| | | GCACCGACTTCACCCTGACCATCAGCAGCCTGCAGCCCGAGGACGTGGCCAC |
| | | CTACTACTGCGGCGGCTACAAGAGCTACAGCAACGACGGCAACGGCTTCGGC |
| | | GGCGGCACCAAGGTGGAGATCAAGAGGACCGTGGCCGCCCCCAGCGTGTTCA |
| | | TCTTCCCCCCCAGCGACGAGCAGCTGAAGAGCGGCACCGCCAGCGTGGTGTG |
| | | CCTGCTGAACAACTTCTACCCCAGGGAGGCCAAGGTGCAGTGGAAGGTGGAC |
| | | AACGCCCTGCAGAGCGGCAACAGCCAGGAGAGCGTGACCGAGCAGGACAGCA |
| | | AGGACAGCACCTACAGCCTGAGCAGCACCCTGACCCTGAGCAAGGCCGACTA |
| | | CGAGAAGCACAAGGTGTACGCCTGCGAGGTGACCCACCAGGGCCTGAGCAGC |
| | | CCCGTGACCAAGAGCTTCAACAGGGGCGAGTGC |
| LKA-651/NVS2 | Heavy/ | GAGGTGCAGCTGGTGCAGAGCGGCGCCGAGGTGAAGAAGCCCGGCGAGAGCC |
| | SEQ ID | TGAAGATCAGCTGCAAGGGCAGCGGCTACAGCTTCACCAGCTACTGGATCGG |
| | NO: 103 | CTGGGTGAGGCAGATGCCCGGCAAGGGCCTGGAGTGGATGGGCTGGATCGAC |
| | | CCCTACAGGAGCGAGATCAGGTACAGCCCCAGCTTCCAGGGCCAGGTGACCA |
| | | TCAGCGCCGACAAGAGCATCAGCACCGCCTACCTGCAGTGGAGCAGCCTGAA |
| | | GGCCAGCGACACCGCCATGTACTACTGCGCCAGGGTGAGCAGCGAGCCCTTC |
| | | GACAGCTGGGGCCAGGGCACCCTGGTGACCGTGAGCAGCGCCAGCACCAAGG |
| | | GCCCCAGCGTGTTCCCCCTGGCCCCCAGCAGCAAGAGCACCAGCGGCGGCAC |
| | | CGCCGCCCTGGGCTGCCTGGTGAAGGACTACTTCCCCGAGCCCGTGACCGTG |
| | | AGCTGGAACAGCGGCGCCCTGACCAGCGGCGTGCACACCTTCCCCGCCGTGC |
| | | TGCAGAGCAGCGGCCTGTACAGCCTGAGCAGCGTGGTGACCGTGCCCAGCAG |
| | | CAGCCTGGGCACCCAGACCTACATCTGCAACGTGAACCACAAGCCCAGCAAC |
| | | ACCAAGGTGGACAAGAGGGTGGAGCCCAAGAGCTGCGAC +/− AAGACCCA |
| | | CACC (or AAGACCCACCTG) +/− TGCCCCCCCTGCCCCGCC +/− |
| | | CCCGAGCTGCTGGGCGGCCCCAGCGTGTTCCTG |
| LKA-651/NVS2 | Light/ | AGCTACGTGCTGACCCAGCCCCCCAGCGTGAGCGTGGCCCCCGGCAAGACCG |
| | SEQ ID | CCAGGATCACCTGCAGCGGCGACAAGCTGGGCGACCACTACGCCTACTGGTA |
| | NO: 104 | CCAGCAGAAGCCCGGCCAGGCCCCCGTGCTGGTGATCTACGACGACAGCAAG |
| | | AGGCCCAGCGGCATCCCCGAGAGGTTCAGCGGCAGCAACAGCGGCAACACCG |
| | | CCACCCTGACCATCAGCAGGGTGGAGGCCGGCGACGAGGCCGACTACTACTG |
| | | CGCCACCTGGACCTTCGAGGGCGACTACGTGTTCGGCGGCGGCACCAAGCTG |
| | | ACCGTGCTGGGCCAGCCCAAGGCCGCCCCCAGCGTGACCCTGTTCCCCCCCA |
| | | GCAGCGAGGAGCTGCAGGCCAACAAGGCCACCCTGGTGTGCCTGATCAGCGA |
| | | CTTCTACCCCGGCGCCGTGACCGTGGCCTGGAAGGCCGACAGCAGCCCCGTG |
| | | AAGGCCGGCGTGGAGACCACCACCCCCAGCAAGCAGAGCAACAACAAGTACG |
| | | CCGCCAGCAGCTACCTGAGCCTGACCCCCGAGCAGTGGAAGAGCCACAGGAG |
| | | CTACAGCTGCCAGGTGACCCACGAGGGCAGCACCGTGGAGAAGACCGTGGCC |
| | | CCCACCGAGTGCAGC |
| LKA-651/NVS3 | Heavy/ | CAGGTGCAGCTGCAGCAGAGCGGCCCCGGCCTGGTGAAGCCCAGCCAGACCC |
| | SEQ ID | TGAGCCTGACCTGCGCCATCAGCGGCGACAGCGTGAGCAGCAACACCGCCGC |
| | NO: 105 | CTGGAACTGGATCAGGCAGAGCCCCAGCAGGGGCCTGGAGTGGCTGGGCGTG |
| | | ATCTACTACAGGAGCAAGTGGTACAACGACTACGCCGTGAGCGTGAAGAGCA |
| | | GGATCACCATCAACCCCGACACCAGCAAGAACCAGTTCAGCCTGCAGCTGAA |
| | | CAGCGTGACCCCCGAGGACACCGCCGTGTACTACTGCGCCAGGAGCGTGCCC |
| | | GGCGGCGACCCCGGCCTGGAGCACGCCTTCGCCTACTGGGGCAGGGGCACCC |
| | | TGGTGACCGTGAGCAGCGCCAGCACCAAGGGCCCCAGCGTGTTCCCCCTGGC |
| | | CCCCAGCAGCAAGAGCACCAGCGGCGGCACCGCCGCCCTGGGCTGCCTGGTG |
| | | AAGGACTACTTCCCCGAGCCCGTGACCGTGAGCTGGAACAGCGGCGCCCTGA |
| | | CCAGCGGCGTGCACACCTTCCCCGCCGTGCTGCAGAGCAGCGGCCTGTACAG |
| | | CCTGAGCAGCGTGGTGACCGTGCCCAGCAGCAGCCTGGGCACCCAGACCTAC |
| | | ATCTGCAACGTGAACCACAAGCCCAGCAACACCAAGGTGGACAAGAGGGTGG |
| | | AGCCCAAGAGCTGCGAC +/− AAGACCCACACC (or AAGACCCACCTG) |
| | | +/− TGCCCCCCCTGCCCCGCC +/− CCCGAGCTGCTGGGCGGCCCCAG |
| | | CGTGTTCCTG |
| LKA-651/NVS3 | Light/ | AGCTACGTGCTGACCCAGCCCCCCAGCGTGAGCGTGGCCCCCGGCAAGACCG |
| | SEQ ID | CCAGGATCACCTGCAGCGGCGACAACCTGGGCACCTACTACGTGGAGTGGTA |
| | NO: 106 | CCAGCAGAAGCCCGGCCAGGCCCCCGTGCTGGTGATCTACGACGACAGCGAC |
| | | AGGCCCAGCGGCATCCCCGAGAGGTTCAGCGGCAGCAACAGCGGCAACACCG |
| | | CCACCCTGACCATCAGCAGGGTGGAGGCCGGCGACGAGGCCGACTACTACTG |
| | | CGCCAGCTTCGCCAGCTGGAGCGACAGCGTGTTCGGCGGCGGCACCAAGCTG |
| | | ACCGTGCTGGGCCAGCCCAAGGCCGCCCCCAGCGTGACCCTGTTCCCCCCCA |
| | | GCAGCGAGGAGCTGCAGGCCAACAAGGCCACCCTGGTGTGCCTGATCAGCGA |
| | | CTTCTACCCCGGCGCCGTGACCGTGGCCTGGAAGGCCGACAGCAGCCCCGTG |
| | | AAGGCCGGCGTGGAGACCACCACCCCCAGCAAGCAGAGCAACAACAAGTACG |
| | | CCGCCAGCAGCTACCTGAGCCTGACCCCCGAGCAGTGGAAGAGCCACAGGAG |
| | | CTACAGCTGCCAGGTGACCCACGAGGGCAGCACCGTGGAGAAGACCGTGGCC |
| | | CCCACCGAGTGCAGC |
| Ascrinvacumab | Heavy/ | ACCTGCACCGTGAGCGGCGGCAGCATCAGCAGCGGCGAGTACTACTGGAACT |
| | SEQ ID | GGATCAGGCAGCACCCCGGCAAGGGCCTGGAGTGGATCGGCTACATCTACTA |
| | NO: 107 | CAGCGGCAGCACCTACTACAACCCCAGCCTGAAGAGCAGGGTGACCATCAGC |
| | | GTGGACACCAGCAAGAACCAGTTCAGCCTGAAGCTGAGCAGCGTGACCGCCG |
| | | CCGACACCGCCGTGTACTACTGCGCCAGGGAGAGCGTGGCCGGCTTCGACTA |
| | | CTGGGGCCAGGGCACCCTGGTGACCGTGAGCAGCGCCAGCACCAAGGGCCCC |
| | | AGCGTGTTCCCCCTGGCCCCCTGCAGCAGGAGCACCAGCGAGAGCACCGCCG |
| | | CCCTGGGCTGCCTGGTGAAGGACTACTTCCCCGAGCCCGTGACCGTGAGCTG |
| | | GAACAGCGGCGCCCTGACCAGCGGCGTGCACACCTTCCCCGCCGTGCTGCAG |
| | | AGCAGCGGCCTGTACAGCCTGAGCAGCGTGGTGACCGTGCCCAGCAGCAACT |
| | | TCGGCACCCAGACCTACACCTGCAACGTGGACCACAAGCCCAGCAACACCAA |
| | | GGTGGACAAGACCGTGGAGAGGAAGTGCTGCGTGGAG +/− |
| | | TGCCCCCCCTGCCCCGCC +/− CCCCCCGTGGCCGGC |
| Ascrinvacumab | Light/ | GAGATCGTGCTGACCCAGAGCCCCGGCACCCTGAGCCTGAGCCCCGGCGAGA |
| | SEQ ID | GGGCCACCCTGAGCTGCAGGGCCAGCCAGAGCGTGAGCAGCAGCTACCTGGC |
| | NO: 108 | CTGGTACCAGCAGAAGCCCGGCCAGGCCCCCAGGCTGCTGATCTACGGCACC |
| | | AGCAGCAGGGCCACCGGCATCCCCGACAGGTTCAGCGGCAGCGGCAGCGGCA |
| | | CCGACTTCACCCTGACCATCAGCAGGCTGGAGCCCGAGGACTTCGCCGTGTA |
| | | CTACTGCCAGCAGTACGGCAGCAGCCCCATCACCTTCGGCCAGGGCACCAGG |
| | | CTGGAGATCAAGAGGACCGTGGCCGCCCCCAGCGTGTTCATCTTCCCCCCCA |
| | | GCGACGAGCAGCTGAAGAGCGGCACCGCCAGCGTGGTGTGCCTGCTGAACAA |
| | | CTTCTACCCCAGGGAGGCCAAGGTGCAGTGGAAGGTGGACAACGCCCTGCAG |
| | | AGCGGCAACAGCCAGGAGAGCGTGACCGAGCAGGACAGCAAGGACAGCACCT |
| | | ACAGCCTGAGCAGCACCCTGACCCTGAGCAAGGCCGACTACGAGAAGCACAA |
| | | GGTGTACGCCTGCGAGGTGACCCACCAGGGCCTGAGCAGCCCCGTGACCAAG |
| | | AGCTTCAACAGGGGCGAGTGC |
| Tesidolumab | Heavy/ | GAGGTGCAGCTGGTGCAGAGCGGCGCCGAGGTGAAGAAGCCCGGCAGCAGCG |
| | SEQ ID | TGAAGGTGAGCTGCAAGGCCAGCGGCGGCACCTTCAGCAGCTACGCCATCAG |
| | NO: 109 | CTGGGTGAGGCAGGCCCCCGGCCAGGGCCTGGAGTGGATGGGCGGCATCGGC |
| | | CCCTTCTTCGGCACCGCCAACTACGCCCAGAAGTTCCAGGGCAGGGTGACCA |
| | | TCACCGCCGACGAGAGCACCAGCACCGCCTACATGGAGCTGAGCAGCCTGAG |
| | | GAGCGAGGACACCGCCGTGTACTACTGCGCCAGGGACACCCCCTACTTCGAC |
| | | TACTGGGGCCAGGGCACCCTGGTGACCGTGAGCAGCGCCAGCACCAAGGGCC |
| | | CCAGCGTGTTCCCCCTGGCCCCCAGCAGCAAGAGCACCAGCGGCGGCACCGC |
| | | CGCCCTGGGCTGCCTGGTGAAGGACTACTTCCCCGAGCCCGTGACCGTGAGC |
| | | TGGAACAGCGGCGCCCTGACCAGCGGCGTGCACACCTTCCCCGCCGTGCTGC |
| | | AGAGCAGCGGCCTGTACAGCCTGAGCAGCGTGGTGACCGTGCCCAGCAGCAG |
| | | CCTGGGCACCCAGACCTACATCTGCAACGTGAACCACAAGCCCAGCAACACC |
| | | AAGGTGGACAAGAGGGTGGAGCCCAAGAGCTGCGAC +/− AAGACCCACACC |
| | | (or AAGACCCACCTG) +/− TGCCCCCCCTGCCCCGCC +/− |
| | | CCCGAGGCCGCCGGCGGCCCCAGCGTGTTCCTG |
| Tesidolumab | Light/ | AGCTACGAGCTGACCCAGCCCCTGAGCGTGAGCGTGGCCCTGGGCCAGACCG |
| | SEQ ID | CCAGGATCACCTGCAGCGGCGACAGCATCCCCAACTACTACGTGTACTGGTA |
| | NO: 110 | CCAGCAGAAGCCCGGCCAGGCCCCCGTGCTGGTGATCTACGACGACAGCAAC |
| | | AGGCCCAGCGGCATCCCCGAGAGGTTCAGCGGCAGCAACAGCGGCAACACCG |
| | | CCACCCTGACCATCAGCAGGGCCCAGGCCGGCGACGAGGCCGACTACTACTG |
| | | CCAGAGCTTCGACAGCAGCCTGAACGCCGAGGTGTTCGGCGGCGGCACCAAG |
| | | CTGACCGTGCTGGGCCAGCCCAAGGCCGCCCCCAGCGTGACCCTGTTCCCCC |
| | | CCAGCAGCGAGGAGCTGCAGGCCAACAAGGCCACCCTGGTGTGCCTGATCAG |
| | | CGACTTCTACCCCGGCGCCGTGACCGTGGCCTGGAAGGCCGACAGCAGCCCC |
| | | GTGAAGGCCGGCGTGGAGACCACCACCCCCAGCAAGCAGAGCAACAACAAGT |
| | | ACGCCGCCAGCAGCTACCTGAGCCTGACCCCCGAGCAGTGGAAGAGCCACAG |
| | | GAGCTACAGCTGCCAGGTGACCCACGAGGGCAGCACCGTGGAGAAGACCGTG |
| | | GCCCCCACCGAGTGCAGC |
| Carotuximab | Heavy/ | GAGGTGAAGCTGGAGGAGAGCGGCGGCGGCCTGGTGCAGCCCGGCGGCAGCA |
| | SEQ ID | TGAAGCTGAGCTGCGCCGCCAGCGGCTTCACCTTCAGCGACGCCTGGATGGA |
| | NO: 111 | CTGGGTGAGGCAGAGCCCCGAGAAGGGCCTGGAGTGGGTGGCCGAGATCAGG |
| | | AGCAAGGCCAGCAACCACGCCACCTACTACGCCGAGAGCGTGAAGGGCAGGT |
| | | TCACCATCAGCAGGGACGACAGCAAGAGCAGCGTGTACCTGCAGATGAACAG |
| | | CCTGAGGGCCGAGGACACCGGCATCTACTACTGCACCAGGTGGAGGAGGTTC |
| | | TTCGACAGCTGGGGCCAGGGCACCACCCTGACCGTGAGCAGCGCCAGCACCA |
| | | AGGGCCCCAGCGTGTTCCCCCTGGCCCCCAGCAGCAAGAGCACCAGCGGCGG |
| | | CACCGCCGCCCTGGGCTGCCTGGTGAAGGACTACTTCCCCGAGCCCGTGACC |
| | | GTGAGCTGGAACAGCGGCGCCCTGACCAGCGGCGTGCACACCTTCCCCGCCG |
| | | TGCTGCAGAGCAGCGGCCTGTACAGCCTGAGCAGCGTGGTGACCGTGCCCAG |
| | | CAGCAGCCTGGGCACCCAGACCTACATCTGCAACGTGAACCACAAGCCCAGC |
| | | AACACCAAGGTGGACAAGAAGGTGGAGCCCAAGAGCTGCGAC +/− |
| | | AAGACCCACACC (or AAGACCCACCTG) +/− |
| | | TGCCCCCCCTGCCCCGCC +/− CCCGAGCTGCTGGGCGGCCCCAGCGTGTTC |
| | | CTG |
| Carotuximab | Light/ | CAGATCGTGCTGAGCCAGAGCCCCGCCATCCTGAGCGCCAGCCCCGGCGAGA |
| | SEQ ID | AGGTGACCATGACCTGCAGGGCCAGCAGCAGCGTGAGCTACATGCACTGGTA |
| | NO: 112 | CCAGCAGAAGCCCGGCAGCAGCCCCAAGCCCTGGATCTACGCCACCAGCAAC |
| | | CTGGCCAGCGGCGTGCCCGTGAGGTTCAGCGGCAGCGGCAGCGGCACCAGCT |
| | | ACAGCCTGACCATCAGCAGGGTGGAGGCCGAGGACGCCGCCACCTACTACTG |
| | | CCAGCAGTGGAGCAGCAACCCCCTGACCTTCGGCGCCGGCACCAAGCTGGAG |
| | | CTGAAGAGGACCGTGGCCGCCCCCAGCGTGTTCATCTTCCCCCCCAGCGACG |
| | | AGCAGCTGAAGAGCGGCACCGCCAGCGTGGTGTGCCTGCTGAACAACTTCTA |
| | | CCCCAGGGAGGCCAAGGTGCAGTGGAAGGTGGACAACGCCCTGCAGAGCGGC |
| | | AACAGCCAGGAGAGCGTGACCGAGCAGGACAGCAAGGACAGCACCTACAGCC |
| | | TGAGCAGCACCCTGACCCTGAGCAAGGCCGACTACGAGAAGCACAAGGTGTA |
| | | CGCCTGCGAGGTGACCCACCAGGGCCTGAGCAGCCCCGTGACCAAGAGCTTC |
| | | AACAGGGGCGAGTGC |
| ANX-007 | Heavy/ | CAGGTGCAGCTGGTGCAGAGCGGCGCCGAGCTGAAGAAGCCCGGCGCCAGCG |
| | SEQ ID | TGAAGGTGAGCTGCAAGAGCAGCGGCTACCACTTCACCAGCTACTGGATGCA |
| | NO: 113 | CTGGGTGAAGCAGGCCCCCGGCCAGGGCCTGGAGTGGATCGGCGTGATCCAC |
| | | cCCAACAGCGGCAGCATCAACTACAACGAGAAGTTCGAGAGCAGGGTGACCA |
| | | TCACCGTGGACAAGAGCACCAGCACCGCCTACATGGAGCTGAGCAGCCTGAG |
| | | GAGCGAGGACACCGCCGTGTACTACTGCGCCGGCGAGAGGGACAGCACCGAG |
| | | GTGCTGCCCATGGACTACTGGGGCCAGGGCACCACCGTGACCGTGAGCAGCG |
| | | CCAGCACCAAGGGCCCCAGCGTGTTCCCCCTGGCCCCCAGCAGCAAGAGCAC |
| | | CAGCGGCGGCACCGCCGCCCTGGGCTGCCTGGTGAAGGACTACTTCCCCGAG |
| | | CCCGTGACCGTGAGCTGGAACAGCGGCGCCCTGACCAGCGGCGTGCACACCT |
| | | TCCCCGCCGTGCTGCAGAGCAGCGGCCTGTACAGCCTGAGCAGCGTGGTGAC |
| | | CGTGCCCAGCAGCAGCCTGGGCACCCAGACCTACATCTGCAACGTGAACCAC |
| | | AAGCCCAGCAACACCAAGGTGGACAAGAGGGTGGAGCCCAAGAGCTGCGAC |
| | | +/− AAGACCCACACC (or AAGACCCACCTG) +/− |
| | | TGCCCCCCCTGCCCCGCC +/− CCCGAGCTGCTGGGCGGCCCCAGCGTGTT |
| | | CCTG |
| ANX-007 | Light/ | GACGTGCAGATCACCCAGAGCCCCAGCAGCCTGAGCGCCAGCCTGGGCGAGA |
| | SEQ ID | GGGCCACCATCAACTGCAGGGCCAGCAAGAGCATCAACAAGTACCTGGCCTG |
| | NO: 114 | GTACCAGCAGAAGCCCGGCAAGGCCCCCAAGCTGCTGATCTACAGCGGCAGC |
| | | ACCCTGCAGAGCGGCATCCCCGCCAGGTTCAGCGGCAGCGGCAGCGGCACCG |
| | | ACTTCACCCTGACCATCAGCAGCCTGGAGCCCGAGGACTTCGCCATGTACTA |
| | | CTGCCAGCAGCACAACGAGTACCCCCTGACCTTCGGCCAGGGCACCAAGCTG |
| | | GAGATCAAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| Adalimumab | Heavy/ | GAGGTGCAGCTGGTGGAGAGCGGCGGCGGCCTGGTGCAGCCCGGCAGGAGCC |
| | SEQ ID | TGAGGCTGAGCTGCGCCGCCAGCGGCTTCACCTTCGACGACTACGCCATGCA |
| | NO: 115 | CTGGGTGAGGCAGGCCCCCGGCAAGGGCCTGGAGTGGGTGAGCGCCATCACC |
| | | TGGAACAGCGGCCACATCGACTACGCCGACAGCGTGGAGGGCAGGTTCACCA |
| | | TCAGCAGGGACAACGCCAAGAACAGCCTGTACCTGCAGATGAACAGCCTGAG |
| | | GGCCGAGGACACCGCCGTGTACTACTGCGCCAAGGTGAGCTACCTGAGCACC |
| | | GCCAGCAGCCTGGACTACTGGGGCCAGGGCACCCTGGTGACCGTGAGCAGCG |
| | | CCAGCACCAAGGGCCCCAGCGTGTTCCCCCTGGCCCCCAGCAGCAAGAGCAC |
| | | CAGCGGCGGCACCGCCGCCCTGGGCTGCCTGGTGAAGGACTACTTCCCCGAG |
| | | CCCGTGACCGTGAGCTGGAACAGCGGCGCCCTGACCAGCGGCGTGCACACCT |
| | | TCCCCGCCGTGCTGCAGAGCAGCGGCCTGTACAGCCTGAGCAGCGTGGTGAC |
| | | CGTGCCCAGCAGCAGCCTGGGCACCCAGACCTACATCTGCAACGTGAACCAC |
| | | AAGCCCAGCAACACCAAGGTGGACAAGAAGGTGGAGCCCAAGAGCTGCGAC |
| | | +/− AAGACCCACACC (or AAGACCCACCTG) +/− |
| | | TGCCCCCCCTGCCCCGCC +/− CCCGAGCTGCTGGGCGGCCCCAGCGTGTT |
| | | CCTG |
| Adalimumab | Light/ | GACATCCAGATGACCCAGAGCCCCAGCAGCCTGAGCGCCAGCGTGGGCGACA |
| | SEQ ID | GGGTGACCATCACCTGCAGGGCCAGCCAGGGCATCAGGAACTACCTGGCCTG |
| | NO: 116 | GTACCAGCAGAAGCCCGGCAAGGCCCCCAAGCTGCTGATCTACGCCGCCAGC |
| | | ACCCTGCAGAGCGGCGTGCCCAGCAGGTTCAGCGGCAGCGGCAGCGGCACCG |
| | | ACTTCACCCTGACCATCAGCAGCCTGCAGCCCGAGGACGTGGCCACCTACTA |
| | | CTGCCAGAGGTACAACAGGGCCCCCTACACCTTCGGCCAGGGCACCAAGGTG |
| | | GAGATCAAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| Infliximab | Heavy/ | GAGGTGAAGCTGGAGGAGAGCGGCGGCGGCCTGGTGCAGCCCGGCGGCAGCA |
| | SEQ ID | TGAAGCTGAGCTGCGTGGCCAGCGGCTTCATCTTCAGCAACCACTGGATGAA |
| | NO: 117 | CTGGGTGAGGCAGAGCCCCGAGAAGGGCCTGGAGTGGGTGGCCGAGATCAGG |
| | | AGCAAGAGCATCAACAGCGCCACCCACTACGCCGAGAGCGTGAAGGGCAGGT |
| | | TCACCATCAGCAGGGACGACAGCAAGAGCGCCGTGTACCTGCAGATGACCGA |
| | | CCTGAGGACCGAGGACACCGGCGTGTACTACTGCAGCAGGAACTACTACGGC |
| | | AGCACCTACGACTACTGGGGCCAGGGCACCACCCTGACCGTGAGCAGCGCCA |
| | | GCACCAAGGGCCCCAGCGTGTTCCCCCTGGCCCCCAGCAGCAAGAGCACCAG |
| | | CGGCGGCACCGCCGCCCTGGGCTGCCTGGTGAAGGACTACTTCCCCGAGCCC |
| | | GTGACCGTGAGCTGGAACAGCGGCGCCCTGACCAGCGGCGTGCACACCTTCC |
| | | CCGCCGTGCTGCAGAGCAGCGGCCTGTACAGCCTGAGCAGCGTGGTGACCGT |
| | | GCCCAGCAGCAGCCTGGGCACCCAGACCTACATCTGCAACGTGAACCACAAG |
| | | CCCAGCAACACCAAGGTGGACAAGAAGGTGGAGCCCAAGAGCTGCGAC +/− |
| | | AAGACCCACACC (or AAGACCCACCTG) +/− |
| | | TGCCCCCCCTGCCCCGCC +/− |
| | | CCCGAGCTGCTGGGCGGCCCCAGCGTGTTCCTG |
| Infliximab | Light/ | GACATCCTGCTGACCCAGAGCCCCGCCATCCTGAGCGTGAGCCCCGGCGAGA |
| | SEQ ID | GGGTGAGCTTCAGCTGCAGGGCCAGCCAGTTCGTGGGCAGCAGCATCCACTG |
| | NO: 118 | GTACCAGCAGAGGACCAACGGCAGCCCCAGGCTGCTGATCAAGTACGCCAGC |
| | | GAGAGCATGAGCGGCATCCCCAGCAGGTTCAGCGGCAGCGGCAGCGGCACCG |
| | | ACTTCACCCTGAGCATCAACACCGTGGAGAGCGAGGACATCGCCGACTACTA |
| | | CTGCCAGCAGAGCCACAGCTGGCCCTTCACCTTCGGCAGCGGCACCAACCTG |
| | | GAGGTGAAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| Golimumab | Heavy/ | AGCAAGCTGCAGGTGCAGCTGGTGGAGAGCGGCGGCGGCGTGGTGCAGCCCG |
| | SEQ ID | GCAGGAGCCTGAGGCTGAGCTGCGCCGCCAGCGGCTTCATCTTCAGCAGCTA |
| | NO: 119 | CGCCATGCACTGGGTGAGGCAGGCCCCCGGCAACGGCCTGGAGTGGGTGGCC |
| | | TTCATGAGCTACGACGGCAGCAACAAGAAGTACGCCGACAGCGTGAAGGGCA |
| | | GGTTCACCATCAGCAGGGACAACAGCAAGAACACCCTGTACCTGCAGATGAA |
| | | CAGCCTGAGGGCCGAGGACACCGCCGTGTACTACTGCGCCAGGGACAGGGGC |
| | | ATCGCCGCCGGCGGCAACTACTACTACTACGGCATGGACGTGTGGGGCCAGG |
| | | GCACCACCGTGACCGTGAGCAGCGCCAGCACCAAGGGCCCCAGCGTGTTCCC |
| | | CCTGGCCCCCAGCAGCAAGAGCACCAGCGGCGGCACCGCCGCCCTGGGCTGC |
| | | CTGGTGAAGGACTACTTCCCCGAGCCCGTGACCGTGAGCTGGAACAGCGGCG |
| | | CCCTGACCAGCGGCGTGCACACCTTCCCCGCCGTGCTGCAGAGCAGCGGCCT |
| | | GTACAGCCTGAGCAGCGTGGTGACCGTGCCCAGCAGCAGCCTGGGCACCCAG |
| | | ACCTACATCTGCAACGTGAACCACAAGCCCAGC +/− AAGACCCACACC |
| | | (or AAGACCCACCTG) +/− TGCCCCCCCTGCCCCGCC +/− |
| | | CCCGAGCTGCTGGGCGGCCCCAGCGTGTTCCTG |
| Golimumab | Light/ | GCCGGCAGCGAGATCGTGCTGACCCAGAGCCCCGCCACCCTGAGCCTGAGCC |
| | SEQ ID | CCGGCGAGAGGGCCACCCTGAGCTGCAGGGCCAGCCAGAGCGTGTACAGCTA |
| | NO: 120 | CCTGGCCTGGTACCAGCAGAAGCCCGGCCAGGCCCCCAGGCTGCTGATCTAC |
| | | GACGCCAGCAACAGGGCCACCGGCATCCCCGCCAGGTTCAGCGGCAGCGGCA |
| | | GCGGCACCGACTTCACCCTGACCATCAGCAGCCTGGAGCCCGAGGACTTCGC |
| | | CGTGTACTACTGCCAGCAGAGGAGCAACTGGCCCCCCTTCACCTTCGGCCCC |
| | | GGCACCAAGGTGGACATCAAGAGGACCGTGGCCGCCCCCAGCGTGTTCATCT |
| | | TCCCCCCCAGCGACGAGCAGCTGAAGAGCGGCACCGCCAGCGTGGTGTGCCT |
| | | GCTGAACAACTTCTACCCCAGGGAGGCCAAGGTGCAGTGGAAGGTGGACAAC |
| | | GCCCTGCAGAGCGGCAACAGCCAGGAGAGCGTGACCGAGCAGGACAGCAAGG |
| | | ACAGCACCTACAGCCTGAGCAGCACCCTGACCCTGAGCAAGGCCGACTACGA |
| | | GAAGCACAAGGTGTACGCCTGCGAGGTGACCCACCAGGGCCTGAGCAGCCCC |
| | | GTGACCAAGAGCTTCAACAGGGGCGAGTGC |
| Elezanumab | Heavy/ | GAGGTGCAGCTGGTGCAGAGCGGCGCCGAGGTGAAGAAGCCCGGCGCCAGCG |
| | SEQ ID | TGAAGGTGAGCTGCAAGGCCAGCGGCTACACCTTCACCAGCCACGGCATCAG |
| | NO: 121 | CTGGGTGAGGCAGGCCCCCGGCCAGGGCCTGGACTGGATGGGCTGGATCAGC |
| | | CCCTACAGCGGCAACACCAACTACGCCCAGAAGCTGCAGGGCAGGGTGACCA |
| | | TGACCACCGACACCAGCACCAGCACCGCCTACATGGAGCTGAGCAGCCTGAG |
| | | GAGCGAGGACACCGCCGTGTACTACTGCGCCAGGGTGGGCAGCGGCCCCTAC |
| | | TACTACATGGACGTGTGGGGCCAGGGCACCCTGGTGACCGTGAGCAGCGCCA |
| | | GCACCAAGGGCCCCAGCGTGTTCCCCCTGGCCCCCAGCAGCAAGAGCACCAG |
| | | CGGCGGCACCGCCGCCCTGGGCTGCCTGGTGAAGGACTACTTCCCCGAGCCC |
| | | GTGACCGTGAGCTGGAACAGCGGCGCCCTGACCAGCGGCGTGCACACCTTCC |
| | | CCGCCGTGCTGCAGAGCAGCGGCCTGTACAGCCTGAGCAGCGTGGTGACCGT |
| | | GCCCAGCAGCAGCCTGGGCACCCAGACCTACATCTGCAACGTGAACCACAAG |
| | | CCCAGCAACACCAAGGTGGACAAGAAGGTGGAGCCCAAGAGCTGCGAC +/− |
| | | AAGACCCACACC (or AAGACCCACCTG) +/− |
| | | TGCCCCCCCTGCCCCGCC +/− |
| | | CCCGAGGCCGCCGGCGGCCCCAGCGTGTTCCTG |
| Elezanumab | Light/ | CAGAGCGCCCTGACCCAGCCCAGGAGCGTGAGCGGCAGCCCCGGCCAGAGCG |
| | SEQ ID | TGACCATCAGCTGCACCGGCACCAGCAGCAGCGTGGGCGACAGCATCTACGT |
| | NO: 122 | GAGCTGGTACCAGCAGCACCCCGGCAAGGCCCCCAAGCTGATGCTGTACGAC |
| | | GTGACCAAGAGGCCCAGCGGCGTGCCCGACAGGTTCAGCGGCAGCAAGAGCG |
| | | GCAACACCGCCAGCCTGACCATCAGCGGCCTGCAGGCCGAGGACGAGGCCGA |
| | | CTACTACTGCTACAGCTACGCCGGCACCGACACCCTGTTCGGCGGCGGCACC |
| | | AAGGTGACCGTGCTGGGCCAGCCCAAGGCCGCCCCCAGCGTGACCCTGTTCC |
| | | CCCCCAGCAGCGAGGAGCTGCAGGCCAACAAGGCCACCCTGGTGTGCCTGAT |
| | | CAGCGACTTCTACCCCGGCGCCGTGACCGTGGCCTGGAAGGCCGACAGCAGC |
| | | CCCGTGAAGGCCGGCGTGGAGACCACCACCCCCAGCAAGCAGAGCAACAACA |
| | | AGTACGCCGCCAGCAGCTACCTGAGCCTGACCCCCGAGCAGTGGAAGAGCCA |
| | | CAGGAGCTACAGCTGCCAGGTGACCCACGAGGGCAGCACCGTGGAGAAGACC |
| | | GTGGCCCCCACCGAGTGCAGC |
| NI-301 | Heavy/ | CAGGTGCAGCTGCAGGAGAGCGGCCCCGGCCTGGTGAAGCCCAGCGAGACCC |
| | SEQ ID | TGAGCCTGACCTGCAGCGTGAGCGGCGGCAGCATCATCAGCAGGAGCAGCTA |
| | NO: 123 | CTGGGGCTGGATCAGGCAGCCCCCCGGCAAGGGCCTGGAGTGGATCGGCGGC |
| | | ATCTACCACAGCGGCAACACCTACGACAACCCCAGCCTGAAGAGCAGGCTGA |
| | | CCATGAGCGTGGACACCAGCAAGAACCAGTTCAGCCTGAACCTGAGGAGCGT |
| | | GACCGCCGCCGACACCGCCGTGTACTACTGCGCCAGGATCGTGCCCGGCGGC |
| | | GACGCCTTCGACATCTGGGGCCAGGGCACCATGGTGACCGTGAGCAGCGCCA |
| | | GCACCAAGGGCCCCAGCGTGTTCCCCCTGGCCCCCAGCAGCAAGAGCACCAG |
| | | CGGCGGCACCGCCGCCCTGGGCTGCCTGGTGAAGGACTACTTCCCCGAGCCC |
| | | GTGACCGTGAGCTGGAACAGCGGCGCCCTGACCAGCGGCGTGCACACCTTCC |
| | | CCGCCGTGCTGCAGAGCAGCGGCCTGTACAGCCTGAGCAGCGTGGTGACCGT |
| | | GCCCAGCAGCAGCCTGGGCACCCAGACCTACATCTGCAACGTGAACCACAAG |
| | | CCCAGCAACACCAAGGTGGACAAGAGGGTGGAGCCCAAGAGCTGCGAC +/− |
| | | AAGACCCACACC (or AAGACCCACCTG) +/− |
| | | TGCCCCCCCTGCCCCGCC +/− CCCGAGCTGCTGGGCGGCCCCAGCGTG |
| | | TTCCTG |
| NI-301 | Light/ | GACATCCAGATGACCCAGAGCCCCAGCAGCCTGAGCGCCAGCGTGGGCGACA |
| | SEQ ID | GGGTGACCGCCTGCAGGGCCAGCCAGAGCGTGGGCACCTACCTGAACTGGTA |
| | NO: 124 | CCAGCAGAAGAGGGGCAAGGCCCCCAAGCTGCTGATCTTCGCCGCCAGCAGC |
| | | CTGCAGAGCGGCGTGCCCAGCAGGTTCAGCGGCAGCGGCAGCGGCACCGACT |
| | | TCACCCTGACCATCAGCAGCCTGCAGCCCGAGGACTTCGCCACCTACTACTG |
| | | CCAGCAGAGCTACAGCAGCCCCCCCACCTTCGGCCAGGGCACCAAGGTGGAG |
| | | ATCAAGAGGACCGTGGCCGCCCCCAGCGTGTTCATCTTCCCCCCCAGCGACG |
| | | AGCAGCTGAAGAGCGGCACCGCCAGCGTGGTGTGCCTGCTGAACAACTTCTA |
| | | CCCCAGGGAGGCCAAGGTGCAGTGGAAGGTGGACAACGCCCTGCAGAGCGGC |
| | | AACAGCCAGGAGAGCGTGACCGAGCAGGACAGCAAGGACAGCACCTACAGCC |
| | | TGAGCAGCACCCTGACCCTGAGCAAGGCCGACTACGAGAAGCACAAGGTGTA |
| | | CGCCTGCGAGGTGACCCACCAGGGCCTGAGCAGCCCCGTGACCAAGAGCTTC |
| | | AACAGGGGCGAGTGC |
| PRX-004 | Heavy/ | GAGGTGCAGCTGGTGGAGAGCGGCGGCGGCCTGATCCAGCCCGGCGGCAGCC |
| | SEQ ID | TGAGGCTGAGCTGCGCCGCCAGCGGCTTCACCTTCAGCAACTACGCCATGAG |
| | NO: 125 | CTGGGTGAGGCAGGCCCCCGGCAAGGGCCTGGAGTGGGTGAGCAGCATCAGC |
| | | AGCGGCGGCAGCACCTACTACCCCGACAGCGTGAAGGGCAGGTTCACCATCA |
| | | GCAGGGACAACAGCAAGAACACCCTGTACCTGCAGATGAACAGCCTGAGGGC |
| | | CGAGGACACCGCCGTGTACTACTGCGCCAGGTACTACTACGGCCAGTACTTC |
| | | GACTTCTGGGGCCAGGGCACCCTGGTGACCGTGAGCAGCGCCAGCACCAAGG |
| | | GCCCCAGCGTGTTCCCCCTGGCCCCCAGCAGCAAGAGCACCAGCGGCGGCAC |
| | | CGCCGCCCTGGGCTGCCTGGTGAAGGACTACTTCCCCGAGCCCGTGACCGTG |
| | | AGCTGGAACAGCGGCGCCCTGACCAGCGGCGTGCACACCTTCCCCGCCGTGC |
| | | TGCAGAGCAGCGGCCTGTACAGCCTGAGCAGCGTGGTGACCGTGCCCAGCAG |
| | | CAGCCTGGGCACCCAGACCTACATCTGCAACGTGAACCACAAGCCCAGCAAC |
| | | ACCAAGGTGGACAAGAGGGTGGAGCCCAAGAGCTGCGAC +/− |
| | | AAGACCCACACC (or AAGACCCACCTG) +/− |
| | | TGCCCCCCCTGCCCCGCC +/− CCCGAGCTGCTGGGCGGCCCCAGCGTGTT |
| | | CCTG |
| PRX-004 | Light/ | GACATCGTGATGACCCAGAGCCCCAGCAGCCTGAGCGCCAGCGTGGGCGACA |
| | SEQ ID | GGGTGACCATCACCTGCAAGGCCAGCCAGGACGTGAGCACCACCGTGGCCTG |
| | NO: 126 | GTACCAGCAGAAGCCCGGCAAGGCCCCCAAGCTGCTGATCTACAGCGCCAGC |
| | | TACAGGAGCACCGGCGTGCCCAGCAGGTTCAGCGGCAGCGGCAGCGGCACCG |
| | | ACTTCACCCTGACCATCAGCAGCCTGCAGCCCGAGGACTTCGCCGTGTACTA |
| | | CTGCCAGCAGCACTACAGCACCCCCCTGACCTTCGGCGGCGGCACCAAGGTG |
| | | GAGATCAAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| Pamrevlumab | Heavy/ | GAGGTGCAGCTGGTGCAGAGCGGCGCCGAGGTGAAGAAGCCCGGCAGCAGCG |
| | SEQ ID | TGAAGGTGAGCTGCAAGGCCAGCGGCGGCACCTTCAGCAGCTACGCCATCAG |
| | NO: 127 | CTGGGTGAGGCAGGCCCCCGGCCAGGGCCTGGAGTGGATGGGCGGCATCGGC |
| | | CCCTTCTTCGGCACCGCCAACTACGCCCAGAAGTTCCAGGGCAGGGTGACCA |
| | | TCACCGCCGACGAGAGCACCAGCACCGCCTACATGGAGCTGAGCAGCCTGAG |
| | | GAGCGAGGACACCGCCGTGTACTACTGCGCCAGGGACACCCCCTACTTCGAC |
| | | TACTGGGGCCAGGGCACCCTGGTGACCGTGAGCAGCGCCAGCACCAAGGGCC |
| | | CCAGCGTGTTCCCCCTGGCCCCCAGCAGCAAGAGCACCAGCGGCGGCACCGC |
| | | CGCCCTGGGCTGCCTGGTGAAGGACTACTTCCCCGAGCCCGTGACCGTGAGC |
| | | TGGAACAGCGGCGCCCTGACCAGCGGCGTGCACACCTTCCCCGCCGTGCTGC |
| | | AGAGCAGCGGCCTGTACAGCCTGAGCAGCGTGGTGACCGTGCCCAGCAGCAG |
| | | CCTGGGCACCCAGACCTACATCTGCAACGTGAACCACAAGCCCAGCAACACC |
| | | AAGGTGGACAAGAGGGTGGAGCCCAAGAGCTGCGAC +/− AAGACCCACACC |
| | | (or AAGACCCACCTG) +/− TGCCCCCCCTGCCCCGCC +/− |
| | | CCCGAGGCCGCCGGCGGCCCCAGCGTGTTCCTG |
| Pamrevlumab | Light/ | AGCTACGAGCTGACCCAGCCCCTGAGCGTGAGCGTGGCCCTGGGCCAGACCG |
| | SEQ ID | CCAGGATCACCTGCAGCGGCGACAGCATCCCCAACTACTACGTGTACTGGTA |
| | NO: 128 | CCAGCAGAAGCCCGGCCAGGCCCCCGTGCTGGTGATCTACGACGACAGCAAC |
| | | AGGCCCAGCGGCATCCCCGAGAGGTTCAGCGGCAGCAACAGCGGCAACACCG |
| | | CCACCCTGACCATCAGCAGGGCCCAGGCCGGCGACGAGGCCGACTACTACTG |
| | | CCAGAGCTTCGACAGCAGCCTGAACGCCGAGGTGTTCGGCGGCGGCACCAAG |
| | | CTGACCGTGCTGGGCCAGCCCAAGGCCGCCCCCAGCGTGACCCTGTTCCCCC |
| | | CCAGCAGCGAGGAGCTGCAGGCCAACAAGGCCACCCTGGTGTGCCTGATCAG |
| | | CGACTTCTACCCCGGCGCCGTGACCGTGGCCTGGAAGGCCGACAGCAGCCCC |
| | | GTGAAGGCCGGCGTGGAGACCACCACCCCCAGCAAGCAGAGCAACAACAAGT |
| | | ACGCCGCCAGCAGCTACCTGAGCCTGACCCCCGAGCAGTGGAAGAGCCACAG |
| | | GAGCTACAGCTGCCAGGTGACCCACGAGGGCAGCACCGTGGAGAAGACCGTG |
| | | GCCCCCACCGAGTGCAGC |
| Satralizumab | Heavy/ | CAGGTGCAGCTGCAGGAGAGCGGCCCCGGCCTGGTGAAGCCCAGCGAGACCC |
| | SEQ ID | TGAGCCTGACCTGCGCCGTGAGCGGCCACAGCATCAGCCACGACCACGCCTG |
| | NO: 129 | GAGCTGGGTGAGGCAGCCCCCCGGCGAGGGCCTGGAGTGGATCGGCTTCATC |
| | | AGCTACAGCGGCATCACCAACTACAACCCCAGCCTGCAGGGCAGGGTGACCA |
| | | TCAGCAGGGACAACAGCAAGAACACCCTGTACCTGCAGATGAACAGCCTGAG |
| | | GGCCGAGGACACCGCCGTGTACTACTGCGCCAGGAGCCTGGCCAGGACCACC |
| | | GCCATGGACTACTGGGGCGAGGGCACCCTGGTGACCGTGAGCAGCGCCAGCA |
| | | CCAAGGGCCCCAGCGTGTTCCCCCTGGCCCCCAGCAGCAAGAGCACCAGCGG |
| | | CGGCACCGCCGCCCTGGGCTGCCTGGTGAAGGACTACTTCCCCGAGCCCGTG |
| | | ACCGTGAGCTGGAACAGCGGCGCCCTGACCAGCGGCGTGCACACCTTCCCCG |
| | | CCGTGCTGCAGAGCAGCGGCCTGTACAGCCTGAGCAGCGTGGTGACCGTGCC |
| | | CAGCAGCAACTTCGGCACCCAGACCTACACCTGCAACGTGGACCACAAGCCC |
| | | AGCAACACCAAGGTGGACAAGACCGTGGAGAGGAAGAGCTGCGTGGAG |
| | | +/− TGCCCCCCCTGCCCCGCC +/− CCCCCCGTGGCCGGC |
| Satralizumab | Light/ | GACATCCAGATGACCCAGAGCCCCAGCAGCCTGAGCGCCAGCGTGGGCGACA |
| | SEQ ID | GCGTGACCATCACCTGCCAGGCCAGCACCGACATCAGCAGCCACCTGAACTG |
| | NO: 130 | GTACCAGCAGAAGCCCGGCAAGGCCCCCGAGCTGCTGATCTACTACGGCAGC |
| | | CACCTGCTGAGCGGCGTGCCCAGCAGGTTCAGCGGCAGCGGCAGCGGCACCG |
| | | ACTTCACCTTCACCATCAGCAGCCTGGAGGCCGAGGACGCCGCCACCTACTA |
| | | CTGCGGCCAGGGCAACAGGCTGCCCTACACCTTCGGCCAGGGCACCAAGGTG |
| | | GAGATCGAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| Sarilumab | Heavy/ | GAGGTGCAGCTGGTGGAGAGCGGCGGCGGCCTGGTGCAGCCCGGCAGGAGCC |
| | SEQ ID | TGAGGCTGAGCTGCGCCGCCAGCAGGTTCACCTTCGACGACTACGCCATGCA |
| | NO: 131 | CTGGGTGAGGCAGGCCCCCGGCAAGGGCCTGGAGTGGGTGAGCGGCATCAGC |
| | | TGGAACAGCGGCAGGATCGGCTACGCCGACAGCGTGAAGGGCAGGTTCACCA |
| | | TCAGCAGGGACAACGCCGAGAACAGCCTGTTCCTGCAGATGAACGGCCTGAG |
| | | GGCCGAGGACACCGCCCTGTACTACTGCGCCAAGGGCAGGGACAGCTTCGAC |
| | | ATCTGGGGCCAGGGCACCATGGTGACCGTGAGCAGCGCCAGCACCAAGGGCC |
| | | CCAGCGTGTTCCCCCTGGCCCCCAGCAGCAAGAGCACCAGCGGCGGCACCGC |
| | | CGCCCTGGGCTGCCTGGTGAAGGACTACTTCCCCGAGCCCGTGACCGTGAGC |
| | | TGGAACAGCGGCGCCCTGACCAGCGGCGTGCACACCTTCCCCGCCGTGCTGC |
| | | AGAGCAGCGGCCTGTACAGCCTGAGCAGCGTGGTGACCGTGCCCAGCAGCAG |
| | | CCTGGGCACCCAGACCTACATCTGCAACGTGAACCACAAGCCCAGCAACACC |
| | | AAGGTGGACAAGAAGGTGGAGCCCAAGAGCTGCGAC +/− AAGACCCACACC |
| | | (or AAGACCCACCTG) +/− TGCCCCCCCTGCCCCGCC +/− |
| | | CCCGAGCTGCTGGGCGGCCCCAGCGTGTTCCTG |
| Sarilumab | Light/ | GACATCCAGATGACCCAGAGCCCCAGCAGCGTGAGCGCCAGCGTGGGCGACA |
| | SEQ ID | GGGTGACCATCACCTGCAGGGCCAGCCAGGGCATCAGCAGCTGGCTGGCCTG |
| | NO: 132 | GTACCAGCAGAAGCCCGGCAAGGCCCCCAAGCTGCTGATCTACGGCGCCAGC |
| | | AGCCTGGAGAGCGGCGTGCCCAGCAGGTTCAGCGGCAGCGGCAGCGGCACCG |
| | | ACTTCACCCTGACCATCAGCAGCCTGCAGCCCGAGGACTTCGCCAGCTACTA |
| | | CTGCCAGCAGGCCAACAGCTTCCCCTACACCTTCGGCCAGGGCACCAAGCTG |
| | | GAGATCAAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| Inebilizumab | Heavy/ | GAGGTGCAGCTGGTGGAGAGCGGCGGCGGCCTGGTGCAGCCCGGCGGCAGCC |
| | SEQ ID | TGAGGCTGAGCTGCGCCGCCAGCGGCTTCACCTTCAGCAGCAGCTGGATGAA |
| | NO: 133 | CTGGGTGAGGCAGGCCCCCGGCAAGGGCCTGGAGTGGGTGGGCAGGATCTAC |
| | | CCCGGCGACGGCGACACCAACTACAACGTGAAGTTCAAGGGCAGGTTCACCA |
| | | TCAGCAGGGACGACAGCAAGAACAGCCTGTACCTGCAGATGAACAGCCTGAA |
| | | GACCGAGGACACCGCCGTGTACTACTGCGCCAGGAGCGGCTTCATCACCACC |
| | | GTGAGGGACTTCGACTACTGGGGCCAGGGCACCCTGGTGACCGTGAGCAGCG |
| | | CCAGCACCAAGGGCCCCAGCGTGTTCCCCCTGGCCCCCAGCAGCAAGAGCAC |
| | | CAGCGGCGGCACCGCCGCCCTGGGCTGCCTGGTGAAGGACTACTTCCCCGAG |
| | | CCCGTGACCGTGAGCTGGAACAGCGGCGCCCTGACCAGCGGCGTGCACACCT |
| | | TCCCCGCCGTGCTGCAGAGCAGCGGCCTGTACAGCCTGAGCAGCGTGGTGAC |
| | | CGTGCCCAGCAGCAGCCTGGGCACCCAGACCTACATCTGCAACGTGAACCAC |
| | | AAGCCCAGCAACACCAAGGTGGACAAGAGGGTGGAGCCCAAGAGCTGCGAC |
| | | +/− AAGACCCACACC (or AAGACCCACCTG) +/− |
| | | TGCCCCCCCTGCCCCGCC +/− CCCGAGCTGCTGGGCGGCCCCAGCGTGT |
| | | TCCTG |
| Inebilizumab | Light/ | GAGATCGTGCTGACCCAGAGCCCCGACTTCCAGAGCGTGACCCCCAAGGAGA |
| | SEQ ID | AGGTGACCATCACCTGCAGGGCCAGCGAGAGCGTGGACACCTTCGGCATCAG |
| | NO: 134 | CTTCATGAACTGGTTCCAGCAGAAGCCCGACCAGAGCCCCAAGCTGCTGATC |
| | | CACGAGGCCAGCAACCAGGGCAGCGGCGTGCCCAGCAGGTTCAGCGGCAGCG |
| | | GCAGCGGCACCGACTTCACCCTGACCATCAACAGCCTGGAGGCCGAGGACGC |
| | | CGCCACCTACTACTGCCAGCAGAGCAAGGAGGTGCCCTTCACCTTCGGCGGC |
| | | GGCACCAAGGTGGAGATCAAGAGGACCGTGGCCGCCCCCAGCGTGTTCATCT |
| | | TCCCCCCCAGCGACGAGCAGCTGAAGAGCGGCACCGCCAGCGTGGTGTGCCT |
| | | GCTGAACAACTTCTACCCCAGGGAGGCCAAGGTGCAGTGGAAGGTGGACAAC |
| | | GCCCTGCAGAGCGGCAACAGCCAGGAGAGCGTGACCGAGCAGGACAGCAAGG |
| | | ACAGCACCTACAGCCTGAGCAGCACCCTGACCCTGAGCAAGGCCGACTACGA |
| | | GAAGCACAAGGTGTACGCCTGCGAGGTGACCCACCAGGGCCTGAGCAGCCCC |
| | | GTGACCAAGAGCTTCAACAGGGGCGAGTGC |
| Etrolizumab | Heavy/ | GAGGTGCAGCTGGTGGAGAGCGGCGGCGGCCTGGTGCAGCCCGGCGGCAGCC |
| | SEQ ID | TGAGGCTGAGCTGCGCCGCCAGCGGCTTCTTCATCACCAACAACTACTGGGG |
| | NO: 135 | CTGGGTGAGGCAGGCCCCCGGCAAGGGCCTGGAGTGGGTGGGCTACATCAGC |
| | | TACAGCGGCAGCACCAGCTACAACCCCAGCCTGAAGAGCAGGTTCACCATCA |
| | | GCAGGGACACCAGCAAGAACACCTTCTACCTGCAGATGAACAGCCTGAGGGC |
| | | CGAGGACACCGCCGTGTACTACTGCGCCAGGACCGGCAGCAGCGGCTACTTC |
| | | GACTTCTGGGGCCAGGGCACCCTGGTGACCGTGAGCAGCGCCAGCACCAAGG |
| | | GCCCCAGCGTGTTCCCCCTGGCCCCCAGCAGCAAGAGCACCAGCGGCGGCAC |
| | | CGCCGCCCTGGGCTGCCTGGTGAAGGACTACTTCCCCGAGCCCGTGACCGTG |
| | | AGCTGGAACAGCGGCGCCCTGACCAGCGGCGTGCACACCTTCCCCGCCGTGC |
| | | TGCAGAGCAGCGGCCTGTACAGCCTGAGCAGCGTGGTGACCGTGCCCAGCAG |
| | | CAGCCTGGGCACCCAGACCTACATCTGCAACGTGAACCACAAGCCCAGCAAC |
| | | ACCAAGGTGGACAAGAAGGTGGAGCCCAAGAGCTGCGAC +/− |
| | | AAGACCCACACC (or AAGACCCACCTG) +/− |
| | | TGCCCCCCCTGCCCCGCC +/− CCCGAGCTGCTGGGCGGCCCCAGCGTGT |
| | | TCCTG |
| Etrolizumab | Light/ | GACATCCAGATGACCCAGAGCCCCAGCAGCCTGAGCGCCAGCGTGGGCGACA |
| | SEQ ID | GGGTGACCATCACCTGCAGGGCCAGCGAGAGCGTGGACGACCTGCTGCACTG |
| | NO: 136 | GTACCAGCAGAAGCCCGGCAAGGCCCCCAAGCTGCTGATCAAGTACGCCAGC |
| | | CAGAGCATCAGCGGCGTGCCCAGCAGGTTCAGCGGCAGCGGCAGCGGCACCG |
| | | ACTTCACCCTGACCATCAGCAGCCTGCAGCCCGAGGACTTCGCCACCTACTA |
| | | CTGCCAGCAGGGCAACAGCCTGCCCAACACCTTCGGCCAGGGCACCAAGGTG |
| | | GAGATCAAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| Romosozumab | Heavy/ | GAGGTGCAGCTGGTGCAGAGCGGCGCCGAGGTGAAGAAGCCCGGCGCCAGCG |
| | SEQ ID | TGAAGGTGAGCTGCAAGGCCAGCGGCTACACCTTCACCGACTACAACATGCA |
| | NO: 137 | CTGGGTGAGGCAGGCCCCCGGCCAGGGCCTGGAGTGGATGGGCGAGATCAAC |
| | | CCCAACAGCGGCGGCGCCGGCTACAACCAGAAGTTCAAGGGCAGGGTGACCA |
| | | TGACCACCGACACCAGCACCAGCACCGCCTACATGGAGCTGAGGAGCCTGAG |
| | | GAGCGACGACACCGCCGTGTACTACTGCGCCAGGCTGGGCTACGACGACATC |
| | | TACGACGACTGGTACTTCGACGTGTGGGGCCAGGGCACCACCGTGACCGTGA |
| | | GCAGCGCCAGCACCAAGGGCCCCAGCGTGTTCCCCCTGGCCCCCTGCAGCAG |
| | | GAGCACCAGCGAGAGCACCGCCGCCCTGGGCTGCCTGGTGAAGGACTACTTC |
| | | CCCGAGCCCGTGACCGTGAGCTGGAACAGCGGCGCCCTGACCAGCGGCGTGC |
| | | ACACCTTCCCCGCCGTGCTGCAGAGCAGCGGCCTGTACAGCCTGAGCAGCGT |
| | | GGTGACCGTGCCCAGCAGCAACTTCGGCACCCAGACCTACACCTGCAACGTG |
| | | GACCACAAGCCCAGCAACACCAAGGTGGACAAGACCGTGGAGAGGAAGTGCT |
| | | GCGTGGAG +/− TGCCCCCCCTGCCCCGCC +/− CCCCCCGTGGCCGGC |
| Romosozumab | Light/ | GACATCCAGATGACCCAGAGCCCCAGCAGCCTGAGCGCCAGCGTGGGCGACA |
| | SEQ ID | GGGTGACCATCACCTGCAGGGCCAGCCAGGACATCAGCAACTACCTGAACTG |
| | NO: 138 | GTACCAGCAGAAGCCCGGCAAGGCCCCCAAGCTGCTGATCTACTACACCAGC |
| | | AGGCTGCTGAGCGGCGTGCCCAGCAGGTTCAGCGGCAGCGGCAGCGGCACCG |
| | | ACTTCACCCTGACCATCAGCAGCCTGCAGCCCGAGGACTTCGCCACCTACTA |
| | | CTGCCAGCAGGGCGACACCCTGCCCTACACCTTCGGCGGCGGCACCAAGGTG |
| | | GAGATCAAGAGGACCGTGGCCGCCCCCAGCGTGTTCATCTTCCCCCCCAGCG |
| | | ACGAGCAGCTGAAGAGCGGCACCGCCAGCGTGGTGTGCCTGCTGAACAACTT |
| | | CTACCCCAGGGAGGCCAAGGTGCAGTGGAAGGTGGACAACGCCCTGCAGAGC |
| | | GGCAACAGCCAGGAGAGCGTGACCGAGCAGGACAGCAAGGACAGCACCTACA |
| | | GCCTGAGCAGCACCCTGACCCTGAGCAAGGCCGACTACGAGAAGCACAAGGT |
| | | GTACGCCTGCGAGGTGACCCACCAGGGCCTGAGCAGCCCCGTGACCAAGAGC |
| | | TTCAACAGGGGCGAGTGC |
| Lanadelumab | Heavy/ | GAGGTGCAGCTGCTGGAGAGCGGCGGCGGCCTGGTGCAGCCCGGCGGCAGCC |
| | SEQ ID | TGAGGCTGAGCTGCGCCGCCAGCGGCTTCACCTTCAGCCACTACATCATGAT |
| | NO: 139 | GTGGGTGAGGCAGGCCCCCGGCAAGGGCCTGGAGTGGGTGAGCGGCATCTAC |
| | | AGCAGCGGCGGCATCACCGTGTACGCCGACAGCGTGAAGGGCAGGTTCACCA |
| | | TCAGCAGGGACAACAGCAAGAACACCCTGTACCTGCAGATGAACAGCCTGAG |
| | | GGCCGAGGACACCGCCGTGTACTACTGCGCCTACAGGAGGATCGGCGTGCCC |
| | | AGGAGGGACGAGTTCGACATCTGGGGCCAGGGCACCATGGTGACCGTGAGCA |
| | | GCGCCAGCACCAAGGGCCCCAGCGTGTTCCCCCTGGCCCCCAGCAGCAAGAG |
| | | CACCAGCGGCGGCACCGCCGCCCTGGGCTGCCTGGTGAAGGACTACTTCCCC |
| | | GAGCCCGTGACCGTGAGCTGGAACAGCGGCGCCCTGACCAGCGGCGTGCACA |
| | | CCTTCCCCGCCGTGCTGCAGAGCAGCGGCCTGTACAGCCTGAGCAGCGTGGT |
| | | GACCGTGCCCAGCAGCAGCCTGGGCACCCAGACCTACATCTGCAACGTGAAC |
| | | CACAAGCCCAGCAACACCAAGGTGGACAAGAGGGTGGAGCCCAAGAGCTGCG |
| | | AC +/− AAGACCCACACC (or AAGACCCACCTG) +/− |
| | | TGCCCCCCCTGCCCCGCC +/− CCCGAGCTGCTGGGCGGCCCCAGCGTGT |
| | | TCCTG |
| Lanadelumab | Light/ | GACATCCAGATGACCCAGAGCCCCAGCACCCTGAGCGCCAGCGTGGGCGACA |
| | SEQ ID | GGGTGACCATCACCTGCAGGGCCAGCCAGAGCATCAGCAGCTGGCTGGCCTG |
| | NO: 140 | GTACCAGCAGAAGCCCGGCAAGGCCCCCAAGCTGCTGATCTACAAGGCCAGC |
| | | ACCCTGGAGAGCGGCGTGCCCAGCAGGTTCAGCGGCAGCGGCAGCGGCACCG |
| | | AGTTCACCCTGACCATCAGCAGCCTGCAGCCCGACGACTTCGCCACCTACTA |
| | | CTGCCAGCAGTACAACACCTACTGGACCTTCGGCCAGGGCACCAAGGTGGAG |
| | | ATCAAGAGGACCGTGGCCGCCCCCAGCGTGTTCATCTTCCCCCCCAGCGACG |
| | | AGCAGCTGAAGAGCGGCACCGCCAGCGTGGTGTGCCTGCTGAACAACTTCTA |
| | | CCCCAGGGAGGCCAAGGTGCAGTGGAAGGTGGACAACGCCCTGCAGAGCGGC |
| | | AACAGCCAGGAGAGCGTGACCGAGCAGGACAGCAAGGACAGCACCTACAGCC |
| | | TGAGCAGCACCCTGACCCTGAGCAAGGCCGACTACGAGAAGCACAAGGTGTA |
| | | CGCCTGCGAGGTGACCCACCAGGGCCTGAGCAGCCCCGTGACCAAGAGCTTC |
| | | AACAGGGGCGAGTGC |
| Lanadelumab | Light/ | ATGTACCGGATGCAGCTGCTGCTGCTCATTGCCCTGTCTCTGGCCCTGGTCA |
| L01 | SEQ ID | CCAATAGCGAAGTCCAGCTGCTGGAATCTGGCGGCGGACTTGTTCAACCTGG |
| | NO: 141 | CGGCTCTCTGAGACTGAGCTGTGCCGCTTCCGGCTTCACCTTCAGCCACTAT |
| | | ATCATGATGTGGGTCCGACAGGCCCCTGGCAAAGGCCTTGAATGGGTGTCCG |
| | | GCATCTATAGCAGCGGCGGCATCACAGTGTACGCCGACTCTGTGAAGGGCAG |
| | | ATTCACCATCAGCCGGGACAACAGCAAGAACACCCTGTACCTGCAGATGAAC |
| | | AGCCTGAGAGCCGAGGACACCGCCGTGTACTACTGCGCCTATAGAAGAATCG |
| | | GCGTGCCCAGACGGGACGAGTTCGATATTTGGGGCCAGGGCACCATGGTCAC |
| | | CGTGTCTAGCGCCTCTACAAAGGGCCCTAGCGTGTTCCCTCTGGCTCCTAGC |
| | | AGCAAGAGCACAAGCGGAGGAACAGCCGCTCTGGGCTGTCTGGTCAAGGACT |
| | | ACTTTCCCGAGCCTGTGACCGTGTCCTGGAATAGCGGAGCACTGACAAGCGG |
| | | CGTGCACACCTTTCCAGCTGTGCTGCAAAGCAGCGGCCTGTACTCTCTGAGC |
| | | AGCGTGGTCACAGTGCCAAGCTCTAGCCTGGGCACCCAGACCTACATCTGCA |
| | | ATGTGAACCACAAGCCTAGCAACACCAAGGTGGACAAGAGAGTGGAACCCAA |
| | | GAGCTGCGACAAGACCCACACCTGTCCTCCATGTCCTGCTCCAGAACTGCTC |
| | | GGCGGACCTTCCGTGTTCCTGTTTCCTCCAAAGCCTAAGGACACCCTGATGA |
| | | TCAGCAGAACCCCTGAAGTGACCTGCGTGGTGGTGGATGTGTCCCACGAGGA |
| | | TCCCGAAGTGAAGTTCAATTGGTACGTGGACGGCGTGGAAGTGCACAACGCC |
| | | AAGACCAAGCCTAGAGAGGAACAGTACAACAGCACCTACAGAGTGGTGTCCG |
| | | TGCTGACCGTGCTGCACCAGGATTGGCTGAACGGCAAAGAGTACAAGTGCAA |
| | | GGTGTCCAACAAGGCCCTGCCTGCTCCTATCGAGAAAACCATCAGCAAGGCC |
| | | AAGGGCCAGCCTAGGGAACCCCAGGTTTACACACTGCCTCCAAGCCGGGAAG |
| | | AGATGACCAAGAACCAGGTGTCCCTGACCTGCCTCGTGAAGGGCTTCTACCC |
| | | TTCCGATATCGCCGTGGAATGGGAGAGCAATGGCCAGCCAGAGAACAACTAC |
| | | AAGACAACCCCTCCTGTGCTGGACAGCGACGGCTCATTCTTCCTGTACAGCA |
| | | AGCTGACAGTGGACAAGTCCAGATGGCAGCAGGGCAACGTGTTCAGCTGCAG |
| | | CGTGATGCACGAGGCCCTGCACAACCACTACACCCAGAAGAGTCTGAGCCTG |
| | | TCTCCAGGCCTGCGGAAGAGAAGGGCTCCTGTGAAGCAGACCCTGAACTTCG |
| | | ACCTGCTGAAACTGGCCGGCGACGTGGAAAGCAACCCCGGACCTATGTATAG |
| | | AATGCAGCTCCTCCTGCTGATCGCACTGAGCCTGGCTCTCGTGACCAACAGC |
| | | GACATCCAGATGACACAGAGCCCCAGCACACTGTCTGCCAGCGTGGGAGACA |
| | | GAGTGACCATCACCTGTAGAGCCAGCCAGTCCATCTCCTCTTGGCTGGCCTG |
| | | GTATCAGCAAAAGCCTGGCAAGGCCCCTAAGCTGCTGATCTACAAGGCCTCC |
| | | ACACTGGAAAGCGGGGTGCCCTCCAGATTTTCTGGCAGCGGATCTGGCACCG |
| | | AGTTCACCCTGACCATCAGTAGCCTGCAGCCTGACGACTTCGCCACCTACTA |
| | | CTGCCAGCAGTACAATACCTACTGGACCTTCGGCCAGGGAACAAAGGTGGAA |
| | | ATCAAGCGGACTGTGGCCGCTCCAAGCGTGTTCATCTTTCCACCTAGCGACG |
| | | AGCAGCTGAAGTCCGGCACAGCCTCTGTTGTGTGCCTGCTGAACAACTTCTA |
| | | CCCCAGAGAAGCCAAGGTGCAGTGGAAAGTGGACAATGCCCTGCAGAGCGGC |
| | | AACTCCCAAGAGAGCGTGACAGAGCAGGACTCCAAGGATTCCACCTACAGCC |
| | | TGTCTAGCACCCTGACACTGAGCAAGGCCGACTACGAGAAGCACAAAGTGTA |
| | | TGCCTGCGAAGTGACCCACCAGGGCCTTAGCTCTCCAGTGACCAAGAGCTTC |
| | | AACCGGGGCGAGTGTTGATAA |
| Siltuximab | Heavy/ | gaagtgcagctggtggaaagcggcggcaaactgctgaaaccgggcggcagcc |
| | SEQ ID | tgaaactgagctgcgcggcgagcggctttacctttagcagctttgcgatgag |
| | NO: 343 | ctggtttcgccagagcccggaaaaacgcctggaatgggtggcggaaattagc |
| | | agcggcggcagctatacctattatccggataccgtgaccggccgctttacca |
| | | ttagccgcgataacgcgaaaaacaccctgtatctggaaatgagcagcctgcg |
| | | cagcgaagataccgcgatgtattattgcgcgcgcggcctgtggggctattat |
| | | gcgctggattattggggccagggcaccagcgtgaccgtgagcagcgcgagca |
| | | ccaaaggcccgagcgtgtttccgctggcgccgagcagcaaaagcaccagcgg |
| | | cggcaccgcggcgctgggctgcctggtgaaagattattttccggaaccggtg |
| | | accgtgagctggaacagcggcgcgctgaccagcggcgtgcatacctttccgg |
| | | cggtgctgcagagcagcggcctgtatagcctgagcagcgtggtgaccgtgcc |
| | | gagcagcagcctgggcacccagacctatatttgcaacgtgaaccataaaccg |
| | | agcaacaccaaagtggataaaaaagtggaaccgaaaagctgcgat |
| | | +/− AAGACCCACACC (or AAGACCCACCTG) +/− |
| | | TGCCCCCCCTGCCCCGCC +/− |
| | | CCCGAGCTGCTGGGCGGCCCCAGCGTGTTCCTG |
| Siltuximab | Light/ | cagattgtgctgattcagagcccggcgattatgagcgcgagcccgggcgaaa |
| | SEQ ID | aagtgaccatgacctgcagcgcgagcagcagcgtgagctatatgtattggta |
| | NO: 344 | tcagcagaaaccgggcagcagcccgcgcctgctgatttatgataccagcaac |
| | | ctggcgagcggcgtgccggtgcgctttagcggcagcggcagcggcaccagct |
| | | atagcctgaccattagccgcatggaagcggaagatgcggcgacctattattg |
| | | ccagcagtggagcggctatccgtatacctttggcggcggcaccaaactggaa |
| | | attaaacgcaccgtggcggcgccgagcgtgtttatttttccgccgagcgatg |
| | | aacagctgaaaagcggcaccgcgagcgtggtgtgcctgctgaacaactttta |
| | | tccgcgcgaagcgaaagtgcagtggaaagtggataacgcgctgcagagcggc |
| | | aacagccaggaaagcgtgaccgaacaggatagcaaagatagcacctatagcc |
| | | tgagcagcaccctgaccctgagcaaagcggattatgaaaaacataaagtgta |
| | | tgcgtgcgaagtgacccatcagggcctgagcagcccggtgaccaaaagcttt |
| | | aaccgcggcgaatgc |
| Clazakizumab | Heavy/ | gaagtgcagctggtggaaagcggcggcggcctggtgcagccgggcggcagcc |
| | SEQ ID | tgcgcctgagctgcgcggcgagcggctttagcctgagcaactattatgtgac |
| | NO: 345 | ctgggtgcgccaggcgccgggcaaaggcctggaatgggtgggcattatttat |
| | | ggcagcgatgaaaccgcgtatgcgaccagcgcgattggccgctttaccatta |
| | | gccgcgataacagcaaaaacaccctgtatctgcagatgaacagcctgcgcgc |
| | | ggaagataccgcggtgtattattgcgcgcgcgatgatagcagcgattgggat |
| | | gcgaaatttaacctgtggggccagggcaccctggtgaccgtgagcagcgcga |
| | | gcaccaaaggcccgagcgtgtttccgctggcgccgagcagcaaaagcaccag |
| | | cggcggcaccgcggcgctgggctgcctggtgaaagattattttccggaaccg |
| | | gtgaccgtgagctggaacagcggcgcgctgaccagcggcgtgcatacctttc |
| | | cggcggtgctgcagagcagcggcctgtatagcctgagcagcgtggtgaccgt |
| | | gccgagcagcagcctgggcacccagacctatatttgcaacgtgaaccataaa |
| | | ccgagcaacaccaaagtggataaacgcgtggaaccgaaaagctgcgat |
| | | +/− AAGACCCACACC (or AAGACCCACCTG) +/− |
| | | TGCCCCCCCTGCCCCGCC +/− |
| | | CCCGAGCTGCTGGGCGGCCCCAGCGTGTTCCTG |
| Clazakizumab | Light/ | gcgattcagatgacccagagcccgagcagcctgagcgcgagcgtgggcgatc |
| | SEQ ID | gcgtgaccattacctgccaggcgagccagagcattaacaacgaactgagctg |
| | NO: 346 | gtatcagcagaaaccgggcaaagcgccgaaactgctgatttatcgcgcgagc |
| | | accctggcgagcggcgtgccgagccgctttagcggcagcggcagcggcaccg |
| | | attttaccctgaccattagcagcctgcagccggatgattttgcgacctatta |
| | | ttgccagcagggctatagcctgcgcaacattgataacgcgtttggcggcggc |
| | | accaaagtggaaattaaacgcaccgtggcggcgccgagcgtgtttatttttc |
| | | cgccgagcgatgaacagctgaaaagcggcaccgcgagcgtggtgtgcctgct |
| | | gaacaacttttatccgcgcgaagcgaaagtgcagtggaaagtggataacgcg |
| | | ctgcagagcggcaacagccaggaaagcgtgaccgaacaggatagcaaagata |
| | | gcacctatagcctgagcagcaccctgaccctgagcaaagcggattatgaaaa |
| | | acataaagtgtatgcgtgcgaagtgacccatcagggcctgagcagcccggtg |
| | | accaaaagctttaaccgcggcgaatgcgaaccgaaaagctgcgat |
| | | +/− AAGACCCACACC (or AAGACCCACCTG) +/− |
| | | TGCCCCCCCTGCCCCGCC +/− |
| | | CCCGAGCTGCTGGGCGGCCCCAGCGTGTTCCTG |
| Sirukumab | Heavy/ | Gaagtgcagctggtggaaagcggcggcggcctggtgcagccgggcggcagcc |
| | SEQ ID | tgcgcctgagctgcgcggcgagcggctttacctttagcccgtttgcgatgag |
| | NO: 347 | ctgggtgcgccaggcgccgggcaaaggcctggaatgggtggcgaaaattagc |
| | | ccgggcggcagctggacctattatagcgataccgtgaccggccgctttacca |
| | | ttagccgcgataacgcgaaaaacagcctgtatctgcagatgaacagcctgcg |
| | | cgcggaagataccgcggtgtattattgcgcgcgccagctgtggggctattat |
| | | gcgctggatatttggggccagggcaccaccgtgaccgtgagcagcgcgagca |
| | | ccaaaggcccgagcgtgtttccgctggcgccgagcagcaaaagcaccagcgg |
| | | cggcaccgcggcgctgggctgcctggtgaaagattattttccggaaccggtg |
| | | accgtgagctggaacagcggcgcgctgaccagcggcgtgcatacctttccgg |
| | | cggtgctgcagagcagcggcctgtatagcctgagcagcgtggtgaccgtgcc |
| | | gagcagcagcctgggcacccagacctatatttgcaacgtgaaccataaaccg |
| | | agcaacaccaaagtggataaaaaagtggaaccgaaaagctgcgat |
| | | +/− AAGACCCACACC (or AAGACCCACCTG) +/− |
| | | TGCCCCCCCTGCCCCGCC +/− |
| | | CCCGAGCTGCTGGGCGGCCCCAGCGTGTTCCTG |
| Sirukumab | Light/ | gaaattgtgctgacccagagcccggcgaccctgagcctgagcccgggcgaac |
| | SEQ ID | gcgcgaccctgagctgcagcgcgagcattagcgtgagctatatgtattggta |
| | NO: 348 | tcagcagaaaccgggccaggcgccgcgcctgctgatttatgatatgagcaac |
| | | ctggcgagcggcattccggcgcgctttagcggcagcggcagcggcaccgatt |
| | | ttaccctgaccattagcagcctggaaccggaagattttgcggtgtattattg |
| | | catgcagtggagcggctatccgtatacctttggcggcggcaccaaagtggaa |
| | | attaaacgcaccgtggcggcgccgagcgtgtttatttttccgccgagcgatg |
| | | aacagctgaaaagcggcaccgcgagcgtggtgtgcctgctgaacaactttta |
| | | tccgcgcgaagcgaaagtgcagtggaaagtggataacgcgctgcagagcggc |
| | | aacagccaggaaagcgtgaccgaacaggatagcaaagatagcacctatagcc |
| | | tgagcagcaccctgaccctgagcaaagcggattatgaaaaacataaagtgta |
| | | tgcgtgcgaagtgacccatcagggcctgagcagcccggtgaccaaaagcttt |
| | | aaccgcggcgaatgc |
| Olokizumab | Heavy/ | Gaagtgcagctggtggaaagcggcggcggcctggtgcagccgggcggcagcc |
| | SEQ ID | tgcgcctgagctgcgcggcgagcggctttaactttaacgattattttatgaa |
| | NO: 349 | ctgggtgcgccaggcgccgggcaaaggcctggaatgggtggcgcagatgcgc |
| | | aacaaaaactatcagtatggcacctattatgcggaaagcctggaaggccgct |
| | | ttaccattagccgcgatgatagcaaaaacagcctgtatctgcagatgaacag |
| | | cctgaaaaccgaagataccgcggtgtattattgcgcgcgcgaaagctattat |
| | | ggctttaccagctattggggccagggcaccctggtgaccgtgagcagcgcga |
| | | gcaccaaaggcccgagcgtgtttccgctggcgccgtgcagccgcagcaccag |
| | | cgaaagcaccgcggcgctgggctgcctggtgaaagattattttccggaaccg |
| | | gtgaccgtgagctggaacagcggcgcgctgaccagcggcgtgcatacctttc |
| | | cggcggtgctgcagagcagcggcctgtatagcctgagcagcgtggtgaccgt |
| | | gccgagcagcagcctgggcaccaaaacctatacctgcaacgtggatcataaa |
| | | ccgagcaacaccaaagtggataaacgcgtgGAGAGCAAGTAC +/− |
| | | GGCCCCCCCTGCCCCCCCTGCCCCGCC |
| | | (GGCCCCCCCTGCCCCAGCTGCCCCGCC) +/− |
| | | CCCGAGTTCCTGGGCGGCCCCAGCGTGTTCCTG |
| Olokizumab | Light/ | gatattcagatgacccagagcccgagcagcctgagcgcgagcgtgggcgatc |
| | SEQ ID | gcgtgaccattacctgccaggcgagccaggatattggcattagcctgagctg |
| | NO: 350 | gtatcagcagaaaccgggcaaagcgccgaaactgctgatttataacgcgaac |
| | | aacctggcggatggcgtgccgagccgctttagcggcagcggcagcggcaccg |
| | | attttaccctgaccattagcagcctgcagccggaagattttgcgacctatta |
| | | ttgcctgcagcataacagcgcgccgtatacctttggccagggcaccaaactg |
| | | gaaattaaacgcaccgtggcggcgccgagcgtgtttatttttccgccgagcg |
| | | atgaacagctgaaaagcggcaccgcgagcgtggtgtgcctgctgaacaactt |
| | | ttatccgcgcgaagcgaaagtgcagtggaaagtggataacgcgctgcagagc |
| | | ggcaacagccaggaaagcgtgaccgaacaggatagcaaagatagcacctata |
| | | gcctgagcagcaccctgaccctgagcaaagcggattatgaaaaacataaagt |
| | | gtatgcgtgcgaagtgacccatcagggcctgagcagcccggtgaccaaaagc |
| | | tttaaccgcggcgaatgc |
| Gerilimzumab | Heavy/ | Gaagtgcagctgcaggaaagcggcccgggcctggtgaaaccgagccagaccc |
| | SEQ ID | tgagcctgacctgcaccgtgagcggcggcagcattaccagccgctattatgc |
| | NO: 351 | gtggagctggattcgccagccgccgggcaaaggcctggaatggattggcgtg |
| | | attgattatgatggcgatacctattatagcccgagcctgaaaagccgcgtga |
| | | gcattagctgggataccagcaaaaaccagtttagcctgaaactgagcagcgt |
| | | gacccoggcggataccgcggtgtattattgcgcgcgcgatccggatgtggtg |
| | | accggctttcattatgattattggggccagggcaccatggtgaccgtgagca |
| | | gcgcgagcaccaaaggcccgagcgtgtttccgctggcgccgagcagcaaaag |
| | | caccagcggcggcaccgcggcgctgggctgcctggtgaaagattattttccg |
| | | gaaccggtgaccgtgagctggaacagcggcgcgctgaccagcggcgtgcata |
| | | cctttccggcggtgctgcagagcagcggcctgtatagcctgagcagcgtggt |
| | | gaccgtgccgagcagcagcctgggcacccagacctatatttgcaacgtgaac |
| | | cataaaccgagcaacaccaaagtggataaaaaagtggaaccgaaaagctgcg |
| | | at +/− AAGACCCACACC (or AAGACCCACCTG) +/− |
| | | TGCCCCCCCTGCCCCGCC +/− |
| | | CCCGAGCTGCTGGGCGGCCCCAGCGTGTTCCTG |
| Gerilimzumab | Light/ | cagagcgcgctgacccagccgccgagcgtgagcggcaccccgggccagagcg |
| | SEQ ID | tgaccattagctgcgcgggcgcgaacaacgatattggcacctatgcgtatgt |
| | NO: 352 | gagctggtatcagcagctgccgggcaccgcgccgaaactgatgatttataaa |
| | | gtgaccacccgcgcgagcggcattccggatcgctttagcggcagcaaaagcg |
| | | gcaacaccgcgagcctgaccattagcggcctgcaggcggaagatgaagcgga |
| | | ttattattgcgcgagctatcgcaactttaacaacgcggtgtttggcaccggc |
| | | accaaactgaccgtgctgggccagccgaaagcggcgccgagcgtgaccctgt |
| | | ttccgccgagcagcgaagaactgcaggcgaacaaagcgaccctggtgtgcct |
| | | gattagcgatttttatccgggcgcggtgaccgtggcgtggaaagcggatagc |
| | | agcccggtgaaagcgggcgtggaaaccaccaccccgagcaaacagagcaaca |
| | | acaaatatgcggcgagcagctatctgagcctgaccccggaacagtggaaaag |
| | | ccatcgcagctatagctgccaggtgacccatgaaggcagcaccgtggaaaaa |
| | | accgtggcgccgaccgaatgcagc |
| Tocilizumab | Heavy/ | Caggtgcagctgcaggaaagcggcccgggcctggtgcgcccgagccagaccc |
| | SEQ ID | tgagcctgacctgcaccgtgagcggctatagcattaccagcgatcatgcgtg |
| | NO: 353 | gagctgggtgcgccagccgccgggccgcggcctggaatggattggctatatt |
| | | agctatagcggcattaccacctataacccgagcctgaaaagccgcgtgacca |
| | | tgctgcgcgataccagcaaaaaccagtttagcctgcgcctgagcagcgtgac |
| | | cgcggcggataccgcggtgtattattgcgcgcgcagcctggcgcgcaccacc |
| | | gcgatggattattggggccagggcagcctggtgaccgtgagcagcgcgagca |
| | | ccaaaggcccgagcgtgtttccgctggcgccgagcagcaaaagcaccagcgg |
| | | cggcaccgoggcgctgggctgcctggtgaaagattattttccggaaccggtg |
| | | accgtgagctggaacagcggcgcgctgaccagcggcgtgcatacctttccgg |
| | | cggtgctgcagagcagcggcctgtatagcctgagcagcgtggtgaccgtgcc |
| | | gagcagcagcctgggcacccagacctatatttgcaacgtgaaccataaaccg |
| | | agcaacaccaaagtggataaaaaagtggaaccgaaaagctgcgat +/− |
| | | AAGACCCACACC (or AAGACCCACCTG) +/− |
| | | TGCCCCCCCTGCCCCGCC +/− |
| | | CCCGAGCTGCTGGGCGGCCCCAGCGTGTTCCTG |
| Tocilizumab | Light/ | gatattcagatgacccagagcccgagcagcctgagcgcgagcgtgggcgatc |
| | SEQ ID | gcgtgaccattacctgccgcgcgagccaggatattagcagctatctgaactg |
| | NO: 354 | gtatcagcagaaaccgggcaaagcgccgaaactgctgatttattataccagc |
| | | cgcctgcatagcggcgtgccgagccgctttagcggcagcggcagcggcaccg |
| | | attttacctttaccattagcagcctgcagccggaagatattgcgacctatta |
| | | ttgccagcagggcaacaccctgccgtatacctttggccagggcaccaaagtg |
| | | gaaattaaacgcaccgtggcggcgccgagcgtgtttatttttccgccgagcg |
| | | atgaacagctgaaaagcggcaccgcgagcgtggtgtgcctgctgaacaactt |
| | | ttatccgcgcgaagcgaaagtgcagtggaaagtggataacgcgctgcagagc |
| | | ggcaacagccaggaaagcgtgaccgaacaggatagcaaagatagcacctata |
| | | gcctgagcagcaccctgaccctgagcaaagcggattatgaaaaacataaagt |
| | | gtatgcgtgcgaagtgacccatcagggcctgagcagcccggtgaccaaaagc |
| | | tttaaccgcggcgaatgc |
| Lecanemab | Heavy/ | Gaagtgcagctggtggaaagcggcggcggcctggtgcagccgggcggcagcc |
| | SEQ ID | tgcgcctgagctgcagcgcgagcggctttacctttagcagctttggcatgca |
| | NO: 376 | ttgggtgcgccaggcgccgggcaaaggcctggaatgggtggcgtatattagc |
| | | agcggcagcagcaccatttattatggcgataccgtgaaaggccgctttacca |
| | | ttagccgcgataacgcgaaaaacagcctgtttctgcagatgagcagcctgcg |
| | | cgcggaagataccgcggtgtattattgcgcgcgcgaaggcggctattattat |
| | | ggccgcagctattataccatggattattggggccagggcaccaccgtgaccg |
| | | tgagcagcgcgagcaccaaaggcccgagcgtgtttccgctggcgccgagcag |
| | | caaaagcaccagcggcggcaccgcggcgctgggctgcctggtgaaagattat |
| | | tttccggaaccggtgaccgtgagctggaacagcggcgcgctgaccagcggcg |
| | | tgcatacctttccggcggtgctgcagagcagcggcctgtatagcctgagcag |
| | | cgtggtgaccgtgccgagcagcagcctgggcacccagacctatatttgcaac |
| | | gtgaaccataaaccgagcaacaccaaagtggataaacgcgtggaaccgaaaa |
| | | gctgcgat +/− AAGACCCACACC (or AAGACCCACCTG) +/− |
| | | TGCCCCCCCTGCCCCGCC +/− |
| | | CCCGAGCTGGCCGGCGCCCCCAGCGTGTTCCTG |
| Lecanemab | Light/ | Gatgtggtgatgacccagagcccgctgagcctgccggtgaccccgggcgcgc |
| | SEQ ID | cggcgagcattagctgccgcagcagccagagcattgtgcatagcaacggcaa |
| | NO: 377 | cacctatctggaatggtatctgcagaaaccgggccagagcccgaaactgctg |
| | | atttataaagtgagcaaccgctttagcggcgtgccggatcgctttagcggca |
| | | gcggcagcggcaccgattttaccctgcgcattagccgcgtggaagcggaaga |
| | | tgtgggcatttattattgctttcagggcagccatgtgccgccgacctttggc |
| | | ccgggcaccaaactggaaattaaacgcaccgtggcggcgccgagcgtgttta |
| | | tttttccgccgagcgatgaacagctgaaaagcggcaccgcgagcgtggtgtg |
| | | cctgctgaacaacttttatccgcgcgaagcgaaagtgcagtggaaagtggat |
| | | aacgcgctgcagagcggcaacagccaggaaagcgtgaccgaacaggatagca |
| | | aagatagcacctatagcctgagcagcaccctgaccctgagcaaagcggatta |
| | | tgaaaaacataaagtgtatgcgtgcgaagtgacccatcagggcctgagcagc |
| | | ccggtgaccaaaagctttaaccgcggcgaatgc |
| Ravulizumab | Heavy/ | caggtgcagctggtgcagagcggcgcggaagtgaaaaaaccgggcgcgagcg |
| | SEQ ID | tgaaagtgagctgcaaagcgagcggccatatttttagcaactattggattca |
| | NO: 378 | gtgggtgcgccaggcgccgggccagggcctggaatggatgggcgaaattctg |
| | | ccgggcagcggccataccgaatataccgaaaactttaaagatcgcgtgacca |
| | | tgacccgcgataccagcaccagcaccgtgtatatggaactgagcagcctgcg |
| | | cagcgaagataccgcggtgtattattgcgcgcgctatttttttggcagcagc |
| | | ccgaactggtattttgatgtgtggggccagggcaccctggtgaccgtgagca |
| | | gcgcgagcaccaaaggcccgagcgtgtttccgctggcgccgtgcagccgcag |
| | | caccagcgaaagcaccgcggcgctgggctgcctggtgaaagattattttccg |
| | | gaaccggtgaccgtgagctggaacagcggcgcgctgaccagcggcgtgcata |
| | | cctttccggcggtgctgcagagcagcggcctgtatagcctgagcagcgtggt |
| | | gaccgtgccgagcagcaactttggcacccagacctatacctgcaacgtggat |
| | | cataaaccgagcaacaccaaagtggataaaaccgtggaacgcaaatgctgcg |
| | | tggaa +/− TGCCCCCCCTGCCCCGCC +/− CCCCCCGTGGCCGGC |
| Ravulizumab | Light/ | Gatattcagatgacccagagcccgagcagcctgagcgcgagcgtgggcgatc |
| | SEQ ID | gcgtgaccattacctgcggcgcgagcgaaaacatttatggcgcgctgaactg |
| | NO: 379 | gtatcagcagaaaccgggcaaagcgccgaaactgctgatttatggcgcgacc |
| | | aacctggcggatggcgtgccgagccgctttagcggcagcggcagcggcaccg |
| | | attttaccctgaccattagcagcctgcagccggaagattttgcgacctatta |
| | | ttgccagaacgtgctgaacaccccgctgacctttggccagggcaccaaagtg |
| | | gaaattaaacgcaccgtggcggcgccgagcgtgtttatttttccgccgagcg |
| | | atgaacagctgaaaagcggcaccgcgagcgtggtgtgcctgctgaacaactt |
| | | ttatccgcgcgaagcgaaagtgcagtggaaagtggataacgcgctgcagagc |
| | | ggcaacagccaggaaagcgtgaccgaacaggatagcaaagatagcacctata |
| | | gcctgagcagcaccctgaccctgagcaaagcggattatgaaaaacataaagt |
| | | gtatgcgtgcgaagtgacccatcagggcctgagcagcccggtgaccaaaagc |
| | | tttaaccgcggcgaatgc |
| Benralizumab | Heavy/ | Gaagtgcagctggtgcagagcggcgcggaagtgaaaaaaccgggcgcgagcg |
| | SEQ ID | tgaaagtgagctgcaaagcgagcggctatacctttaccagctatgtgattca |
| | NO: 380 | ttgggtgcgccagcgcccgggccagggcctggcgtggatgggctatattaac |
| | | ccgtataacgatggcaccaaatataacgaacgctttaaaggcaaagtgacca |
| | | ttaccagcgatcgcagcaccagcaccgtgtatatggaactgagcagcctgcg |
| | | cagcgaagataccgcggtgtatctgtgcggccgcgaaggcattcgctattat |
| | | ggcctgctgggcgattattggggccagggcaccctggtgaccgtgagcagcg |
| | | cgagcaccaaaggcccgagcgtgtttccgctggcgccgagcagcaaaagcac |
| | | cagcggcggcaccgcggcgctgggctgcctggtgaaagattattttccggaa |
| | | ccggtgaccgtgagctggaacagcggcgcgctgaccagcggcgtgcatacct |
| | | ttccggcggtgctgcagagcagcggcctgtatagcctgagcagcgtggtgac |
| | | cgtgccgagcagcagcctgggcacccagacctatatttgcaacgtgaaccat |
| | | aaaccgagcaacaccaaagtggataaaaaagtggaaccgaaaagctgcgat |
| | | +/− AAGACCCACACC (or AAGACCCACCTG) +/− |
| | | TGCCCCCCCTGCCCCGCC +/− |
| | | CCCGAGCTGGCCGGCGCCCCCAGCGTGTTCCTG |
| Benralizumab | Light/ | Gatattcagatgacccagagcccgagcagcctgagcgcgagcgtgggcgatc |
| | SEQ ID | gcgtgaccattacctgcggcaccagcgaagatattattaactatctgaactg |
| | NO: 381 | gtatcagcagaaaccgggcaaagcgccgaaactgctgatttatcataccagc |
| | | cgcctgcagagcggcgtgccgagccgctttagcggcagcggcagcggcaccg |
| | | attttaccctgaccattagcagcctgcagccggaagattttgcgacctatta |
| | | ttgccagcagggctataccctgccgtatacctttggccagggcaccaaagtg |
| | | gaaattaaacgcaccgtggcggcgccgagcgtgtttatttttccgccgagcg |
| | | atgaacagctgaaaagcggcaccgcgagcgtggtgtgcctgctgaacaactt |
| | | ttatccgcgcgaagcgaaagtgcagtggaaagtggataacgcgctgcagagc |
| | | ggcaacagccaggaaagcgtgaccgaacaggatagcaaagatagcacctata |
| | | gcctgagcagcaccctgaccctgagcaaagcggattatgaaaaacataaagt |
| | | gtatgcgtgcgaagtgacccatcagggcctgagcagcccggtgaccaaaagc |
| | | tttaaccgcggcgaatgc |
| Reslizumab | Heavy/ | Gaagtgcagctggtggaaagcggcggcggcctggtgcagccgggcggcagcc |
| | SEQ ID | tgcgcctgagctgcgcggtgagcggcctgagcctgaccagcaacagcgtgaa |
| | NO: 382 | ctggattcgccaggcgccgggcaaaggcctggaatgggtgggcctgatttgg |
| | | agcaacggcgataccgattataacagcgcgattaaaagccgctttaccatta |
| | | gccgcgataccagcaaaagcaccgtgtatctgcagatgaacagcctgcgcgc |
| | | ggaagataccgcggtgtattattgcgcgcgcgaatattatggctattttgat |
| | | tattggggccagggcaccctggtgaccgtgagcagcgcgagcaccaaaggcc |
| | | cgagcgtgtttccgctggcgccgtgcagccgcagcaccagcgaaagcaccgc |
| | | ggcgctgggctgcctggtgaaagattattttccggaaccggtgaccgtgagc |
| | | tggaacagcggcgcgctgaccagcggcgtgcatacctttccggcggtgctgc |
| | | agagcagcggcctgtatagcctgagcagcgtggtgaccgtgccgagcagcag |
| | | cctgggcaccaaaacctatacctgcaacgtggatcataaaccgagcaacacc |
| | | aaagtggataaacgcgtggaaagcaaatatGAGAGCAAGTAC +/− |
| | | GGCCCCCCCTGCCCCCCCTGCCCCGCC |
| | | (GGCCCCCCCTGCCCCAGCTGCCCCGCC) +/− |
| | | CCCGAGTTCCTGGGCGGCCCCAGCGTGTTCCTG |
| Reslizumab | Light/ | Gatattcagatgacccagagcccgagcagcctgagcgcgagcgtgggcgatc |
| | SEQ ID | gcgtgaccattacctgcctggcgagcgaaggcattagcagctatctggcgtg |
| | NO: 383 | gtatcagcagaaaccgggcaaagcgccgaaactgctgatttatggcgcgaac |
| | | agcctgcagaccggcgtgccgagccgctttagcggcagcggcagcgcgaccg |
| | | attataccctgaccattagcagcctgcagccggaagattttgcgacctatta |
| | | ttgccagcagagctataaatttccgaacacctttggccagggcaccaaagtg |
| | | gaagtgaaacgcaccgtggcggcgccgagcgtgtttatttttccgccgagcg |
| | | atgaacagctgaaaagcggcaccgcgagcgtggtgtgcctgctgaacaactt |
| | | ttatccgcgcgaagcgaaagtgcagtggaaagtggataacgcgctgcagagc |
| | | ggcaacagccaggaaagcgtgaccgaacaggatagcaaagatagcacctata |
| | | gcctgagcagcaccctgaccctgagcaaagcggattatgaaaaacataaagt |
| | | gtatgcgtgcgaagtgacccatcagggcctgagcagcccggtgaccaaaagc |
| | | tttaaccgcggcgaatgc |
| Tralokinumab | Heavy/ | Caggtgcagctggtgcagagcggcgcggaagtgaaaaaaccgggcgcgagcg |
| | SEQ ID | tgaaagtgagctgcaaagcgagcggctatacctttaccaactatggcctgag |
| | NO: 384 | ctgggtgcgccaggcgccgggccagggcctggaatggatgggctggattagc |
| | | gcgaacaacggcgataccaactatggccaggaatttcagggccgcgtgacca |
| | | tgaccaccgataccagcaccagcaccgcgtatatggaactgcgcagcctgcg |
| | | cagcgatgataccgcggtgtattattgcgcgcgcgatagcagcagcagctgg |
| | | gcgcgctggttttttgatctgtggggccgcggcaccctggtgaccgtgagca |
| | | gcgcgagcaccaaaggcccgagcgtgtttccgctggcgccgtgcagccgcag |
| | | caccagcgaaagcaccgcggcgctgggctgcctggtgaaagattattttccg |
| | | gaaccggtgaccgtgagctggaacagcggcgcgctgaccagcggcgtgcata |
| | | cctttccggcggtgctgcagagcagcggcctgtatagcctgagcagcgtggt |
| | | gaccgtgccgagcagcagcctgggcaccaaaacctatacctgcaacgtggat |
| | | cataaaccgagcaacaccaaagtggataaacgcgtgGAGAGCAAGTAC |
| | | +/− GGCCCCCCCTGCCCCCCCTGCCCCGCC |
| | | (GGCCCCCCCTGCCCCAGCTGCCCCGCC) +/− |
| | | CCCGAGTTCCTGGGCGGCCCCAGCGTGTTCCTG |
| Tralokinumab | Light/ | Agctatgtgctgacccagccgccgagcgtgagcgtggcgccgggcaaaaccg |
| | SEQ ID | cgcgcattacctgcggcggcaacattattggcagcaaactggtgcattggta |
| | NO: 385 | tcagcagaaaccgggccaggcgccggtgctggtgatttatgatgatggcgat |
| | | cgcccgagcggcattccggaacgctttagcggcagcaacagcggcaacaccg |
| | | cgaccctgaccattagccgcgtggaagcgggcgatgaagcggattattattg |
| | | ccaggtgtgggataccggcagcgatccggtggtgtttggcggcggcaccaaa |
| | | ctgaccgtgctgggccagccgaaagcggcgccgagcgtgaccctgtttccgc |
| | | cgagcagcgaagaactgcaggcgaacaaagcgaccctggtgtgcctgattag |
| | | cgatttttatccgggcgcggtgaccgtggcgtggaaagcggatagcagcccg |
| | | gtgaaagcgggcgtggaaaccaccaccccgagcaaacagagcaacaacaaat |
| | | atgcggcgagcagctatctgagcctgaccccggaacagtggaaaagccatcg |
| | | cagctatagctgccaggtgacccatgaaggcagcaccgtggaaaaaaccgtg |
| | | gcgccgaccgaatgcagc |
| Nemolizumab | Heavy/ | Caggtgcagctggtgcagagcggcgcggaagtgaaaaaaccgggcgcgagcg |
| | SEQ ID | tgaaagtgagctgcaaagcgagcggctatacctttaccggctatattatgaa |
| | NO: 386 | ctgggtgcgccaggcgccgggccagggcctggaatggatgggcctgattaac |
| | | ccgtataacggcggcaccgattataacccgcagtttcaggatcgcgtgacca |
| | | ttaccgcggataaaagcaccagcaccgcgtatatggaactgagcagcctgcg |
| | | cagcgaagataccgcggtgtattattgcgcgcgcgatggctatgatgatggc |
| | | ccgtataccctggaaacctggggccagggcaccctggtgaccgtgagcagcg |
| | | cgagcaccaaaggcccgagcgtgtttccgctggcgccgagcagcaaaagcac |
| | | cagcggcggcaccgcggcgctgggctgcctggtgaaagattattttccggaa |
| | | ccggtgaccgtgagctggaacagcggcgcgctgaccagcggcgtgcatacct |
| | | ttccggcggtgctgcagagcagcggcctgtatagcctgagcagcgtggtgac |
| | | cgtgccgagcagcaactttggcacccagacctatacctgcaacgtggatcat |
| | | aaaccgagcaacaccaaagtggataaaaccgtggaa |
| | | cgcaaaagctgcgtggaa |
| | | +/− TGCCCCCCCTGCCCCGCC +/− CCCCCCGTGGCCGGC |
| Nemolizumab | Light/ | Gatattcagatgacccagagcccgagcagcctgagcgcgagcgtgggcgatc |
| | SEQ ID | gcgtgaccattacctgccaggcgagcgaagatatttatagctttgtggcgtg |
| | NO: 387 | gtatcagcagaaaccgggcaaagcgccgaaactgctgatttataacgcgcag |
| | | accgaagcgcagggcgtgccgagccgctttagcggcagcggcagcggcaccg |
| | | attttaccctgaccattagcagcctgcagccggaagattttgcgacctatta |
| | | ttgccagcatcattatgatagcccgctgacctttggcggcggcaccaaagtg |
| | | gaaattaaacgcaccgtggcggcgccgagcgtgtttatttttccgccgagcg |
| | | atgaacagctgaaaagcggcaccgcgagcgtggtgtgcctgctgaacaactt |
| | | ttatccgcgcgaagcgaaagtgcagtggaaagtggataacgcgctgcagagc |
| | | ggcaacagccaggaaagcgtgaccgaacaggatagcaaagatagcacctata |
| | | gcctgagcagcaccctgaccctgagcaaagcggattatgaaaaacataaagt |
| | | gtatgcgtgcgaagtgacccatcagggcctgagcagcccggtgaccaaaagc |
| | | tttaaccgcggcgaatgc |
| Omalizumab | Heavy/ | Gaagtgcagctggtggaaagcggcggcggcctggtgcagccgggcggcagcc |
| | SEQ ID | tgcgcctgagctgcgcggtgagcggctatagcattaccagcggctatagctg |
| | NO: 388 | gaactggattcgccaggcgccgggcaaaggcctggaatgggtggcgagcatt |
| | | acctatgatggcagcaccaactatgcggatagcgtgaaaggccgctttacca |
| | | ttagccgcgatgatagcaaaaacaccttttatctgcagatgaacagcctgcg |
| | | cgcggaagataccgcggtgtattattgcgcgcgcggcagccattattttggc |
| | | cattggcattttgcggtgtggggccagggcaccctggtgaccgtgagcagcg |
| | | gcccgagcgtgtttccgctggcgccgagcagcaaaagcaccagcggcggcac |
| | | cgcggcgctgggctgcctggtgaaagattattttccggaaccggtgaccgtg |
| | | agctggaacagcggcgcgctgaccagcggcgtgcatacctttccggcggtgc |
| | | tgcagagcagcggcctgtatagcctgagcagcgtggtgaccgtgccgagcag |
| | | cagcctgggcacccagacctatatttgcaacgtgaaccataaaccgagcaac |
| | | accaaagtggataaaaaagcg +/− AAGACCCACACC (or |
| | | AAGACCCACCTG) +/− TGCCCCCCCTGCCCCGCC +/− |
| | | CCCGAGCTGGCCGGCGCCCCCAGCGTGTTCCTG |
| Omalizumab | Light/ | Gatattcagctgacccagagcccgagcagcctgagcgcgagcgtgggcgatc |
| | SEQ ID | gcgtgaccattacctgccgcgcgagccagagcgtggattatgatggcgatag |
| | NO: 389 | ctatatgaactggtatcagcagaaaccgggcaaagcgccgaaactgctgatt |
| | | tatgcggcgagctatctggaaagcggcgtgccgagccgctttagcggcagcg |
| | | gcagcggcaccgattttaccctgaccattagcagcctgcagccggaagattt |
| | | tgcgacctattattgccagcagagccatgaagatccgtatacctttggccag |
| | | ggcaccaaagtggaaattaaacgcaccgtggcggcgccgagcgtgtttattt |
| | | ttccgccgagcgatgaacagctgaaaagcggcaccgcgagcgtggtgtgcct |
| | | gctgaacaacttttatccgcgcgaagcgaaagtgcagtggaaagtggataac |
| | | gcgctgcagagcggcaacagccaggaaagcgtgaccgaacaggatagcaaag |
| | | atagcacctatagcctgagcagcaccctgaccctgagcaaagcggattatga |
| | | aaaacataaagtgtatgcgtgcgaagtgacccatcagggcctgagcagcccg |
| | | gtgaccaaaagctttaaccgcggcgaatgc |
| Tezepelumab | Heavy/ | Cagatgcagctggtggaaagcggcggcggcgtggtgcagccgggccgcagcc |
| | SEQ ID | tgcgcctgagctgcgcggcgagcggctttacctttcgcacctatggcatgca |
| | NO: 390 | ttgggtgcgccaggcgccgggcaaaggcctggaatgggtggcggtgatttgg |
| | | tatgatggcagcaacaaacattatgcggatagcgtgaaaggccgctttacca |
| | | ttacccgcgataacagcaaaaacaccctgaacctgcagatgaacagcctgcg |
| | | cgcggaagataccgcggtgtattattgcgcgcgcgcgccgcagtgggaactg |
| | | gtgcatgaagcgtttgatatttggggccagggcaccatggtgaccgtgagca |
| | | gcgcgagcaccaaaggcccgagcgtgtttccgctggcgccgtgcagccgcag |
| | | caccagcgaaagcaccgcggcgctgggctgcctggtgaaagattattttccg |
| | | gaaccggtgaccgtgagctggaacagcggcgcgctgaccagcggcgtgcata |
| | | cctttccggcggtgctgcagagcagcggcctgtatagcctgagcagcgtggt |
| | | gaccgtgccgagcagcaactttggcacccagacctatacctgcaacgtggat |
| | | cataaaccgagcaacaccaaagtggataaaaccgtg +/− |
| | | TGCCCCCCCTGCCCCGCC +/− CCCCCCGTGGCCGGC |
| Tezepelumab | Light/ | agctatgtgctgacccagccgccgagcgtgagcgtggcgccgggccagaccg |
| | SEQ ID | cgcgcattacctgcggcggcaacaacctgggcagcaaaagcgtgcattggta |
| | NO: 391 | tcagcagaaaccgggccaggcgccggtgctggtggtgtatgatgatagcgat |
| | | cgcccgagctggattccggaacgctttagcggcagcaacagcggcaacaccg |
| | | cgaccctgaccattagccgcggcgaagcgggcgatgaagcggattattattg |
| | | ccaggtgtgggatagcagcagcgatcatgtggtgtttggcggcggcaccaaa |
| | | ctgaccgtgctgggccagccgaaagcggcgccgagcgtgaccctgtttccgc |
| | | cgagcagcgaagaactgcaggcgaacaaagcgaccctggtgtgcctgattag |
| | | cgatttttatccgggcgcggtgaccgtggcgtggaaagcggatagcagcccg |
| | | gtgaaagcgggcgtggaaaccaccaccccgagcaaacagagcaacaacaaat |
| | | atgcggcgagcagctatctgagcctgaccccggaacagtggaaaagccatcg |
| | | cagctatagctgccaggtgacccatgaaggcagcaccgtggaaaaaaccgtg |
| | | gcgccgaccgaatgcagc |

TABLE 7
Table of Fc Domain Amino Acid Sequences

| | Chain/ | |
|---|---|---|
| | SEQ ID | |
| mAb | NO. | Sequence |
| Solanezumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 290 | VSLTCLVKGF YPSDIAVEWE SNGQPENNYK TTPPVLDSDG |
| | | SFFLYSKLTV DKSRWQQGNV FSCSVMHEAL HNHYTQKSLS |
| | | LSPGK |
| GSK933776 | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 291 | VSLTCLVKGF YPSDIAVEWE SNGQPENNYK TTPPVLDSDG |
| | | SFFLYSKLTV DKSRWQQGNV FSCSVMHEAL HNHYTQKSLS |
| | | LSPGK |
| UCB-0107 | Fc | FPPKPKDTLM ISRTPEVTCV VVDVSQEDPE VQFNWYVDGV |
| | domain/ | EVHNAKTKPR EEQFNSTYRV VSVLTVLHQD WLNGKEYKCK |
| | SEQ ID | VSNKGLPSSI EKTISKAKGQ PREPQVYTLP PSQEEMTKNQ |
| | NO: 292 | VSLTCLVKGF YPSDIAVEWE SNGQPENNYK TTPPVLDSDG |
| | | SFFLYSRLTV DKSRWQEGNV FSCSVMHEAL HNHYTQKSLS |
| | | LSLGK |
| Prasinezumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSREEMTKNQ |
| | NO: 293 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| MEDI- | Fc | FPPKPKDTLM ISRTPEVTCV VVDVSHEDPE VKFNWYVDGV |
| 1341/TAK 341 | domain/ | EVHNAKTKPR EEQYNSTYRV VSVLTVLHQD WLNGKEYKCK |
| | SEQ ID | VSNKALPASI EKTISKAKGQ PREPQVYTLP PSREEMTKNQ |
| | NO: 294 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| NI-204.10D12 | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSREEMTKNQ |
| | NO: 295 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Eptinezumab | Fc | FPPKPKDTLM ISRTPEVTCV VVDVSHEDPE VKFNWYVDGV |
| | domain/ | EVHNAKTKPR EEQYASTYRV VSVLTVLHQD WLNGKEYKCK |
| | SEQ ID | VSNKALPAPI EKTISKAKGQ PREPQVYTLP PSREEMTKNQ |
| | NO: 296 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Fremanezumab | Fc | FPPKPKDTLM ISRTPEVTCV VVDVSHEDPE VQFNWYVDGV |
| | domain/ | EVHNAKTKPR EEQFNSTFRV VSVLTVVHQD WLNGKEYKCK |
| | SEQ ID | VSNKGLPSSI EKTISKTKGQ PREPQVYTLP PSREEMTKNQ |
| | NO: 297 | VSLTCLVKGF YPSDIAVEWE SNGQPENNYK TTPPMLDSDG |
| | | SFFLYSKLTV DKSRWQQGNV FSCSVMHEAL HNHYTQKSLS LSPGK |
| Galcanezumab | Fc | FPPKPKDTL MISRTPEVTC VVVDVSQEDP EVQFNWYVDG |
| | domain/ | VEVHNAKTKP REEQFNSTYR VVSVLTVLHQ DWLNGKEYKC |
| | SEQ ID | KVSNKGLPSS IEKTISKAKG QPREPQVYTL PPSQEEMTKN |
| | NO: 298 | QVSLTCLVKG FYPSDIAVEW ESNGQPENNY KTTPPVLDSD |
| | | GSFFLYSRLT VDKSRWQEGN VFSCSVMHEA LHNHYTQKSL SLSLGK |
| Sevacizumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 299 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Ascrinvacumab | Fc | PSVFL FPPKPKDTLM ISRTPEVTCV VVDVSHEDPE VQFNWYVDGV |
| | domain/ | EVHNAKTKPR EEQFNSTFRV VSVLTVVHQD WLNGKEYKCK |
| | SEQ ID | VSNKGLPAPI EKTISKTKGQ PREPQVYTLP PSREEMTKNQ |
| | NO: 300 | VSLTCLVKGF YPSDIAVEWE SNGQPENNYK TTPPMLDSDG |
| | | SFFLYSKLTV DKSRWQQGNV FSCSVMHEAL HNHYTQKSLS LSPGK |
| Tesidolumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSREEMTKNQ |
| | NO: 301 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Carotuximab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 302 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Adalimumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 303 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Infliximab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 304 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Golimumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 305 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Elezanumab | Fc | FPPKPKDQLM ISRTPEVTCV VVDVSHEDPE VKFNWYVDGV EVHNAKTKPR |
| | domain/ | EEQYNSTYRV VSVLTVLHQD WLNGKEYKCK VSNKALPAPI EKTISKAKGQ |
| | SEQ ID | PREPQVYTLP PSREEMTKNQ VSLTCLVKGF YPSDIAVEWE SNGQPENNYK |
| | NO: 306 | TTPPVLDSDG SFFLYSKLTV DKSRWQQGNV FSCSVLHEAL HNHYTQKSLS |
| | | LSPGK |
| PRX-004 | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSREEMTKNQ |
| | NO: 307 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Pamrevlumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSREEMTKNQ |
| | NO: 308 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Satralizumab | Fc | PSVFL FPPKPKDTLM ISRTPEVTCV VVDVSQEDPE VQFNWYVDGV |
| | domain/ | EVHNAKTKPR EEQFNSTFRV VSVLTVVHQD WLNGKEYKCK |
| | SEQ ID | VSNKGLPAPI EKTISKTKGQ PREPQVYTLP PSQEEMTKNQ |
| | NO: 309 | VSLTCLVKGF YPSDIAVEWE SNGQPENNYK TTPPMLDSDG |
| | | SFFLYSKLTV DKSRWQEGNV FSCSVMHEAL HAHYTQKSLS LSP |
| Sarilumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 310 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Inebilizumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSREEMTKNQ |
| | NO: 311 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Etrolizumab | Fc | FPPKPKDTLMI SRTPEVTCVV VDVSHEDPEV KFNWYVDGVE |
| | domain/ | VHNAKTKPRE EQYNSTYRVV SVLTVLHQDW LNGKEYKCKV |
| | SEQ ID | SNKALPAPIE KTISKAKGQP REPQVYTLPP SREEMTKNQV |
| | NO: 312 | SLTCLVKGFY PSDIAVEWES NGQPENNYKT TPPVLDSDGS |
| | | FFLYSKLTVD KSRWQQGNVF SCSVMHEALH NHYTQKSLSL SPG |
| Romosozumab | Fc | PSVFL FPPKPKDTLM ISRTPEVTCV VVDVSHEDPE VQFNWYVDGV |
| | domain/ | EVHNAKTKPR EEQFNSTFRV VSVLTVVHQD WLNGKEYKCK |
| | SEQ ID | VSNKGLPAPI EKTISKTKGQ PREPQVYTLP PSREEMTKNQ |
| | NO:313 | VSLTCLVKGF YPSDIAVEWE SNGQPENNYK TTPPMLDSDG |
| | | SFFLYSKLTV DKSRWQQGNV FSCSVMHEAL HNHYTQKSLS LSPGK |
| Lanadelumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSREEMTKNQ |
| | NO: 314 | VSLTCLVKGF YPSDIAVEWE SNGQPENNYK TTPPVLDSDG |
| | | SFFLYSKLTV DKSRWQQGNV FSCSVMHEAL HNHYTQKSLS LSPG |
| Siltuximab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 355 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Clazakizumab | Fc | FPPKPKDTLM ISRTPEVTCV VVDVSHEDPE VKFNWYVDGV |
| | domain/ | EVHNAKTKPR EEQYASTYRV VSVLTVLHQD WLNGKEYKCK |
| | SEQ ID | VSNKALPAPI EKTISKAKGQ PREPQVYTLP PSREEMTKNQ |
| | NO: 356 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Sirukumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 357 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Olokizumab | Fc | FPPKPKDTLM ISRTPEVTCV VVDVSQEDPE VQFNWYVDGV |
| | domain/ | EVHNAKTKPR EEQFNSTYRV VSVLTVLHQD WLNGKEYKCK |
| | SEQ ID | VSNKGLPSSI EKTISKAKGQ PREPQVYTLP PSQEEMTKNQ |
| | NO: 358 | VSLTCLVKGF YPSDIAVEWE SNGQPENNYK TTPPVLDSDG |
| | | SFFLYSRLTV DKSRWQEGNV FSCSVMHEAL HNHYTQKSLS LSLGK |
| Tocilizumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 359 | VSLTCLVKGF YPSDIAVEWE SNGQPENNYK TTPPVLDSDG |
| | | SFFLYSKLTV DKSRWQQGNV FSCSVMHEAL HNHYTQKSLS LSPG |
| Lecanemab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSREEMTKNQ |
| | No: 392 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Ravulizumab | Fc | FPPKPKDTLM ISRTPEVTCV VVDVSQEDPE VQFNWYVDGV |
| | Domain/ | EVHNAKTKPR EEQFNSTYRV VSVLTVLHQD WLNGKEYKCK |
| | SEQ ID | VSNKGLPSSI EKTISKAKGQ PPEPQVYTLP PSQEEMTKNQ |
| | NO: 393 | VSLTCLVKGF YPSDIAVEWE SNGQPENNYK TTPPVLDSDG |
| | | SFFLYSRLTV DKSRWQEGNV FSCSVLHEAL HSHYTQKSLS LSLGK |
| Benralizumab | Fc | FPPKPKDTLM ISRTPEVTCV VVDVSHEDPE VKFNWYVDGV |
| | Domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 394 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| Reslizumab | Fc | FPPKPKDTLM ISRTPEVTCV VVDVSQEDPE VQFNWYVDGV |
| | Domain/ | EVHNAKTKPR EEQFNSTYRV VSVLTVLHQD WLNGKEYKCK |
| | SEQ ID | VSNKGLPSSI EKTISKAKGQ PREPQVYTLP PSQEEMTKNQ |
| | NO: 395 | VSLTCLVKGF YPSDIAVEWE SNGQPENNYK TTPPVLDSDG |
| | | SFFLYSRLTV DKSRWQEGNV FSCSVMHEAL HNHYTQKSLS LSLGK |
| Tralokinumab | Fc | FPPKPKDTLM ISRTPEVTCV VVDVSQEDPE VQFNWYVDGV |
| | Domain/ | EVHNAKTKPR EEQFNSTYRV VSVLTVLHQD WLNGKEYKCK |
| | SEQ ID | VSNKGLPSSI EKTISKAKGQ PREPQVYTLP PSQEEMTKNQ |
| | NO: 396 | VSLTCLVKGF YPSDIAVEWE SNGQPENNYK TTPPVLDSDG |
| | | SFFLYSRLTV DKSRWQEGNV FSCSVMHEAL HNHYTQKSLS LSLGK |
| Nemolizumab | Fc | FPPKPKDTLM ISRTPEVTCV VVDVSQEDPE VQFNWYVDGV |
| | Domain/ | EVHNAKTKPR EEQFNSTFRV VSVLTVVHQD WLNGKEYKCK |
| | SEQ ID | VSNKGLPAPI EKTISKTKGQ PREPQVYTLP PSQEEMTKNQ |
| | NO: 397 | VSLTCLVKGF YPSDIAVEWE SNGQPENNYK TTPPMLDSDG |
| | | SFFLYSKLTV DKSRWQEGNV FSCSVMHEAL HNHYTQKSLS LSP |
| Omalizumab | Fc | FPPKPKDTLM ISRTPEVTCV VVDVSHEDPE VKFNWYVDGV |
| | Domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 398 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |

### 5.4 Delivery of Gene Therapy Constructs

### 5.4.1 Constructs for Delivery to CNS

Sections 5.3.1, 5.3.2, 5.3.3, 5.3.4, 5.3.5., 5.3.6., and 5.3.7. describe recombinant vectors that contain a transgene encoding a HuPTM mAb or HuPTM Fab (or other antigen binding fragment of the HuPTM mAb) that binds to Aβ, sortilin, Tau protein, SEMA4D, alpha-synuclein, SOD1, and CGRPR, respectively. Such recombinant vector used for delivering the transgene should have a tropism for human CNS cells, such as glial and neuronal cells. Such vectors can include non-replicating recombinant adeno-associated virus vectors (“rAAV”), particularly those bearing an AAV9, AAVrh10, AAVrh20, AAVrh39, or AAVcy5 capsid. However, other viral vectors may be used, including but not limited to lentiviral vectors, vaccinia viral vectors, or non-viral expression vectors referred to as “naked DNA” constructs.

In specific embodiments, provided are constructs for gene therapy administration to a human subject, comprising an AAV vector, which comprises a viral capsid that is at least 95% identical to the amino acid sequence of an AAV9 capsid (SEQ ID NO: 144); and a viral or artificial genome comprising an expression cassette flanked by AAV inverted terminal repeats (ITRs) wherein the expression cassette comprises a transgene encoding the heavy and light chains of the therapeutic antibody, operably linked to one or more regulatory sequences that control expression of the transgene in human cells that express and deliver the therapeutic antibody in a therapeutically appropriate manner as disclosed herein, particularly expressed from CNS cells. In certain embodiments, the encoded AAV9 capsid has the sequence of SEQ ID NO: 144 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions, particularly substitutions with amino acid residues found in the corresponding position in other AAV capsids, for example, in the SUBS row of FIG. 21 which provides a comparison of the amino acid sequences of the capsid sequences of various AAVs, highlighting amino acids appropriate for substitution at different positions within the capsid sequence.

In other specific embodiments, provided are constructs for gene therapy administration to a human subject, comprising an AAV vector, which comprises a viral capsid that is at least 95% identical to the amino acid sequence of an AAVrh10 capsid (SEQ ID NO: 145); and a viral or artificial genome comprising an expression cassette flanked by AAV inverted terminal repeats (ITRs) wherein the expression cassette comprises a transgene encoding the heavy and light chains of the therapeutic antibody, operably linked to one or more regulatory sequences that control expression of the transgene in human cells that express and deliver the therapeutic antibody in a therapeutically appropriate manner as disclosed herein, particularly from CNS cells. In certain embodiments, the encoded AAVrh10 capsid has the sequence of SEQ ID NO: 145 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions, particularly substitutions with amino acid residues found in the corresponding position in other AAV capsids, for example, in the SUBS row of FIG. 21 which provides a comparison of the amino acid sequences of the capsid sequences of various AAVs, highlighting amino acids appropriate for substitution at different positions within the capsid sequence.

In some embodiments, the HuPTM mAb or antigen binding fragment thereof, including the HuPTM Fab transgene should be controlled by appropriate expression control elements for expression of the HuPTM mAb or HuPTM Fab in human CNS cells, for example, the CB7 promoter (a chicken β-actin promoter and CMV enhancer), RSV promoter, GFAP promoter (glial fibrillary acidic protein), MBP promoter (myelin basic protein), MMT promoter, EF-1α, U86 promoter, RPE65 promoter or opsin promoter, an inducible promoter, for example, a hypoxia-inducible promoter or a drug inducible promoter, such as a promoters induced by rapamycin and related agents, and other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal). See, e.g., Powell and Rivera-Soto, 2015, Discov. Med., 19(102):49-57.

Gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. The leader sequence for each of the heavy and light chains is, e.g., MYRMQLLLLIALSLALVTNS (SEQ ID NO: 146) or one of the leader sequences provided in Table 2. Section 5.1.5, supra, provides specific IRES, 2A, and other linker sequences that can be used with the methods and compositions provided herein. In specific embodiments, the linker is a Furin-2A linker, for example a Furin-F2A linker RKRR(GSG)APVKQTLNFDLLKLAGDVESNPGP (SEQ ID NO: 231. In preferred embodiments, the linker is a Furin-2A linker, for example a Furin-T2A linker RKRR(GSG)EGRGSLLTCGDVEENPGP (SEQ ID NO: 429). In specific embodiments, the transgene is a nucleotide sequence that encodes the following: Signal sequence-heavy chain Fab portion-Furin-2A linker-signal sequence-light chain Fab portion. See, e.g., FIGS. 2A-2C for sequences for Solanezumab, lecanemab, and GSK933776 Fab expression, respectively; FIG. 3 for sequence for AL-001 Fab expression; FIGS. 4A-4C for the sequence for ABBV-8E12, UCB-0107, and NI-105 Fab expression, respectively; FIG. 5 for the sequence for VX15/2503 Fab expression; FIGS. 6A-C for the sequence for prasinezumab, NI-202, and MED-1341 Fab expression, respectively; FIGS. 7A-B for the sequence for NI-204 Fab expression; and FIG. 8A-C for the sequence for eptinezumab, fremanezumab, and galcanezumab Fab expression, respectively. In an alternative embodiment, the construct also comprises the nucleotide sequence encoding the Fc domain for the therapeutic antibody (see Table 7 or FIG. 23) for expression of the full length mAb.

In a specific embodiment, the constructs described herein comprise the following components: (1) AAV2 inverted terminal repeats that flank the expression cassette; (2) Control elements, which include a) the CB7 promoter, comprising the CMV enhancer/chicken β-actin promoter, b) a chicken β-actin intron and c) a rabbit 3-globin poly A signal; and (3) nucleic acid sequences coding for the heavy and light chains of the A3-binding, sortilin-binding, Tau protein-binding, SEMA4D-binding, alpha-synuclein-binding, SOD1-binding, or CGRPR-binding Fab, separated by a self-cleaving furin (F)/2A linker, ensuring expression of equal amounts of the heavy and the light chain polypeptides. An exemplary construct is provided in FIG. 1.

In specific embodiments, provided are AAV vectors comprising a viral capsid that is at least 95% identical to the amino acid sequence of an AAV9 capsid (SEQ ID NO: 144) or AAVrh10 (SEQ ID NO: 145); and an artificial genome comprising an expression cassette flanked by AAV inverted terminal repeats (ITRs), wherein the expression cassette comprises a transgene encoding an anti-Aβ, anti-sortilin, anti-Tau protein, anti-SEMA4D, anti-alpha-synuclein, anti-SOD1, or anti-CGRPR mAb, or an antigen-binding fragment thereof, operably linked to one or more regulatory sequences that control expression of the transgene in human CNS cells.

### 5.4.2 Constructs for Delivery to Liver or Muscle Cells

Sections 5.3.8, 5.3.9, 5.3.10, 5.3.11, 5.3.12, 5.3.13, 5.3.14, 5.3.15, 5.3.16, 5.3.17, 5.3.18, 5.3.19, 5.3.20, and 5.3.21 describe recombinant vectors that contain a transgene encoding a HuPTM mAb or HuPTM Fab (or other antigen binding fragment of the HuPTM mAb) that binds VEGF, EpoR, ALK-1, C5, ENG, CC1Q, TNFα, RGMa, TTR, CTGF, IL6R, IL6, CD19, ITGF7, SOST, pKal, IL-6, IL-6R, IL/ILR, IgE, or TSLP. Such recombinant vector used for delivering the transgene can have a tropism for human liver or muscle cells. Such vectors can include non-replicating recombinant adeno-associated virus vectors (“rAAV”), e.g., those bearing an AAV8 or AAV9 capsid. However, other viral vectors may be used, including but not limited to lentiviral vectors, vaccinia viral vectors, or non-viral expression vectors referred to as “naked DNA” constructs.

In specific embodiments, provided are constructs for gene therapy administration to a human subject, comprising an AAV vector, which comprises a viral capsid that is at least 95% identical to the amino acid sequence of an AAV8 capsid (SEQ ID NO: 143); and a viral or artificial genome comprising an expression cassette flanked by AAV inverted terminal repeats (ITRs) wherein the expression cassette comprises a transgene encoding the heavy and light chains of the therapeutic antibody, operably linked to one or more regulatory sequences that control expression of the transgene in human cells (e.g., human muscle or liver cells) that express and deliver the therapeutic antibody in a therapeutically appropriate manner as disclosed herein. In certain embodiments, the encoded AAV8 capsid has the sequence of SEQ ID NO: 143 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions, particularly substitutions with amino acid residues found in the corresponding position in other AAV capsids, for example, in the SUBS row of FIG. 21 which provides a comparison of the amino acid sequences of the capsid sequences of various AAVs, highlighting amino acids appropriate for substitution at different positions within the capsid sequence.

In specific embodiments, provided are constructs for gene therapy administration to a human subject, comprising an AAV vector, which comprises a viral capsid that is at least 95% identical to the amino acid sequence of an AAV9 capsid (SEQ ID NO: 144); and a viral or artificial genome comprising an expression cassette flanked by AAV inverted terminal repeats (ITRs) wherein the expression cassette comprises a transgene encoding the heavy and light chains of the therapeutic antibody, operably linked to one or more regulatory sequences that control expression of the transgene in human cells (e.g., human muscle or liver cells) that express and deliver the therapeutic antibody in a therapeutically appropriate manner as disclosed herein. In certain embodiments, the encoded AAV9 capsid has the sequence of SEQ ID NO: 144 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions, particularly substitutions with amino acid residues found in the corresponding position in other AAV capsids, for example, in the SUBS row of FIG. 21 which provides a comparison of the amino acid sequences of the capsid sequences of various AAVs, highlighting amino acids appropriate for substitution at different positions within the capsid sequence.

In other embodiments, the HuPTM mAb or antigen binding fragment thereof, including the HuPTM Fab transgene should be controlled by appropriate expression control elements for expression of the HuPTM Fab in human liver or muscle cells, for example, the CB7 promoter (a chicken β-actin promoter and CMV enhancer, SEQ ID NO: 411), EF-1 alpha promoter (SEQ ID NO: 415), mU1α (SEQ ID NO: 414), or liver specific promoters such as the TBG (Thyroxine-binding Globulin) promoter (SEQ ID NO: 423), the APOA2 promoter, the SERPINA1 (hAAT) promoter, the ApoE.hAAT promoter (SEQ ID NO: 412), or the MIR122 promoter, or muscle specific promoters, such as the human desmin promoter, the CK8 promoter (SEQ ID NO: 413), or the human Pitx3 promoter, or inducible promoters, such as hypoxia-inducible promoters or rapamycin-inducible promoter, and can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal). See, e.g., Powell and Rivera-Soto, 2015, Discov. Med., 19(102):49-57.

In some embodiments, transgene expression is controlled by tissue-specific promoters or regulatory elements that promote tissue-specificity, for example, for liver-specific expression, LSPX1 (SEQ ID NO: 315), LSPX2 (SEQ ID NO: 316), LTP1 (SEQ ID NO: 317), LTP2 (SEQ ID NO: 318), or LTP3 (SEQ ID NO: 319), for liver and muscle expression, LMTP6 (SEQ ID NO: 320), LMTP13 (SEQ ID NO: 321), LMTP14 (SEQ ID NO: 322), LMTP15 (SEQ ID NO: 323), LMTP18 (SEQ ID NO: 324), LMTP19 (SEQ ID NO: 325), or LMTP20 (SEQ ID NO: 326), or for liver and bone expression, LBTP1 (SEQ ID NO: 327) or LBTP2 (SEQ ID NO: 328), the sequences of which are provided in Table 1.

Gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. The leader sequence for each of the heavy and light chains is, e.g., MYRMQLLLLIALSLALVTNS (SEQ ID NO: 146). Section 5.1.5, supra, provides specific IRES, 2A, and other linker sequences that can be used with the methods and compositions provided herein. In specific embodiments, the linker is a Furin-2A linker, for example, a Furin-F2A linker RKRR(GSG)APVKQTLNFDLLKLAGDVESNPGP (SEQ ID NO: 231) or a Furin-T2A linker RKRR(GSG)EGRGSLLTCGDVEENPGP (SEQ ID NO: 429). In specific embodiments, the transgene is a nucleotide sequence that encodes the following: Signal sequence-heavy chain Fab portion-Furin-2Alinker-signal sequence-light chain Fab portion. See, e.g., FIG. 2B for sequences of GSK933776 Fab expression; FIGS. 9A to 9C for sequences for sevacizumab and LKA-651 Fab expression, respectively; FIGS. 10A to 10D for a sequence for ascrinvacumab, tesidolumab, ravulizumab, and carotuximab Fab expression, respectively; FIG. 11 for sequences for ANX-007 Fab expression; FIGS. 12A to 12C for a sequence for adalimumab, infliximab, and golimumab Fab expression; FIG. 13 for sequence for elezanumab Fab expression; FIGS. 14A and 14B for sequences for NI-301 and PRX-004 Fab expression, respectively; FIG. 15 for a sequence for pamrevlumab Fab expression; FIGS. 16A to 16I for sequences for satralizumab, sarilumab, siltuximab, clazakizumab, sirukumab, olokizumab, gerilimzumab, tocilizumab, and inebilizumab Fab expression, respectively; FIG. 17 for a sequence for etrolizumab Fab expression; FIG. 18 for a sequence for romosozumab Fab expression; FIG. 19 for a sequence for lanadelumab Fab expression; and FIG. 29A to 29F for a sequence for benralizumab, reslizumab, tralokinumab, nemolizumab, omalizumab, and tezepelumab Fab expression respectively. In an alternative embodiment, the construct also comprises the nucleotide sequence encoding the Fc domain for the therapeutic antibody (see Table 7 or FIG. 23) for expression of the full length mAb. In specific embodiments, transgene sequences of expression of lanadelumab are provided in Table 8.

In a specific embodiment, the constructs described herein comprise the following components: (1) AAV2 inverted terminal repeats that flank the expression cassette; (2) Control elements, which include a) an inducible promoter, e.g., a hypoxia-inducible promoter, or a tissue-specific promoter, e.g. ApoE.hAAT, LSPX1, LMTP6, or CK8 or other disclosed in Table 1, b) a chicken β-actin intron and c) a rabbit β-globin poly A signal; and (3) nucleic acid sequences coding for the heavy and light chains of the VEGF-binding, EpoR-binding, ALK-1-binding, C5-binding, ENG-binding, CC1Q-binding, TNF-α binding, RGMa-binding, TTR-binding, CTGF-binding, IL6R-binding, IL6-binding, CD19-binding, ITGF7-binding, SOST-binding, pKal-binding, IL-6 or IL6R-binding, IL/ILR-binding, IgE-binding, or TSLP-binding Fab, separated by a self-cleaving furin (F)/2A linker or T2A linker, ensuring expression of equal amounts of the heavy and the light chain polypeptides. An exemplary construct is provided in FIG. 1.

In a specific embodiment, the constructs described herein comprise the following components: (1) AAV2 inverted terminal repeats that flank the expression cassette; (2) Control elements, which include a) an inducible promoter, e.g., a hypoxia-inducible promoter or a tissue-specific promoter (e.g. ApoE.hAAT or LSPX1), b) a chicken β-actin intron and c) a rabbit β-globin poly A signal; and (3) nucleic acid sequences coding for the heavy and light chains of the VEGF-binding, EpoR-binding, ALK-1-binding, C5-binding, ENG-binding, CC1Q-binding, TNF-α binding, RGMa-binding, TTR-binding, CTGF-binding, IL6R-binding, IL6-binding, CD19-binding, ITGF7-binding, SOST-binding, pKal-binding, IL-6 or IL-6R, IL/ILR-binding, IgE-binding, or TSLP-binding Fab, separated by a self-cleaving furin (F)/2A linker or a T2A linker, ensuring expression of equal amounts of the heavy and the light chain polypeptides. An exemplary construct is provided in FIG. 1.

In specific embodiments, provided are AAV vectors comprising a viral capsid that is at least 95% identical to the amino acid sequence of an AAV8 capsid (SEQ ID NO: 143); and an artificial genome comprising an expression cassette flanked by AAV inverted terminal repeats (ITRs), wherein the expression cassette comprises a transgene encoding an anti-VEGF, anti-EpoR, anti-ALK-1, anti-C5, anti-ENG, anti-CC1Q, anti-TNF-α anti-RGMa, anti-TTR, anti-CTGF, anti-IL6R, anti-IL6, anti-CD19, anti-ITGF7, anti-SOST, anti-pKal, IL-6 or IL-6R, anti-IL/ILR, anti-IgE, or anti-TSLP mAb, or an antigen-binding fragment thereof, operably linked to one or more regulatory sequences that control expression of the transgene in human liver or muscle cells.

In specific embodiments, provided are AAV vectors comprising a viral capsid that is at least 95% identical to the amino acid sequence of an AAV9 (SEQ ID NO: 144); and an artificial genome comprising an expression cassette flanked by AAV inverted terminal repeats (ITRs), wherein the expression cassette comprises a transgene encoding an anti-VEGF, anti-EpoR, anti-ALK-1, anti-C5, anti-ENG, anti-CC1Q, anti-TNF-α anti-RGMa, anti-TTR, anti-CTGF, anti-IL6R, anti-IL6, anti-CD19, anti-ITGF7, anti-SOST, anti-pKal, IL-6 or IL-6R, anti-IL/ILR, anti-IgE, or anti-TSLP mAb, or an antigen-binding fragment thereof, operably linked to one or more regulatory sequences that control expression of the transgene in human muscle cells.

In certain embodiments, a rAAV comprises a transgene encoding an anti-kallikrein antibody, such as lanadelumab, and the construct comprises the following components: (1) AAV2 inverted terminal repeats that flank the expression cassette; (2) regulatory control elements, a) promoter/enhancers, such as any one of Sp7 (SEQ ID NO: 329), minSP7 (SEQ ID NO: 329), ApoE.hAAT (SEQ ID NO: 412), LSPX1 (SEQ ID NO: 315), LSPX2 (SEQ ID NO: 316), LTP1 (SEQ ID NO: 317), LTP2 (SEQ ID NO: 318), LTP3 (SEQ ID NO: 319), LMTP6 (SEQ ID NO: 320), LMTP13 (SEQ ID NO: 321), LMTP14 (SEQ ID NO: 322), LMTP15 (SEQ ID NO: 323), LMTP18 (SEQ ID NO: 324), LMTP19 (SEQ ID NO: 325), LMTP20 (SEQ ID NO: 326), LBTP1 (SEQ ID NO: 327), or LBTP2 (SEQ ID NO: 328) as in Table 1, b) a poly A signal, and c) optionally an intron; and (3) nucleic acid sequences coding for the heavy and light chain of lanadelumab (L01 (SEQ ID NO: 141), L02 (SEQ ID NO: 286), or L03 (SEQ ID NO: 287), Table 8) wherein the heavy chain (Fab and Fc region) and the light chain are separated by a self-cleaving furin (F)/F2A (SEQ ID NO: 231) or furin (F)/T2A (SEQ ID NO: 429) or flexible linker, ensuring expression of equal amounts of the heavy and the light chain polypeptides. In specific embodiments, the transgene expressing lanadelumab has the sequence of SEQ ID NO: 435 to 443.

### 5.4.3 Constructs for Delivery to Retinal Cell Types

Sections 5.3.8, 5.3.9, 5.3.10, 5.3.11, 5.3.15, 5.3.19, and 5.3.20 describe recombinant vectors that contain a transgene encoding a HuPTM mAb or HuPTM Fab (or other antigen binding fragment of the HuPTM mAb) that binds to VEGF, EpoR, Aβ peptides, kallikrein, ALK-1, C5, ENG, CC1Q, TNFαL, IL6R, IL6, and CD19. Such recombinant vectors used for delivering the transgene can have a tropism for one or more human retina cell types. Such vectors can include non-replicating recombinant adeno-associated virus vectors (“rAAV”), e.g., those bearing an AAV8 capsid. Alternatively, an AAV vector bearing an AAV2.7m8 capsid can be used. However, other viral vectors may be used, including but not limited to lentiviral vectors, vaccinia viral vectors, or non-viral expression vectors referred to as “naked DNA” constructs.

In specific embodiments, provided are constructs for gene therapy administration to a human subject, comprising an AAV vector, which comprises a viral capsid that is at least 95% identical to the amino acid sequence of an AAV8 capsid (SEQ ID NO: 143); and a viral or artificial genome comprising an expression cassette flanked by AAV inverted terminal repeats (ITRs) wherein the expression cassette comprises a transgene encoding the heavy and light chains of the therapeutic antibody, operably linked to one or more regulatory sequences that control expression of the transgene in human cells (e.g., retina cell or liver cell types) that express and deliver the therapeutic antibody in a therapeutically appropriate manner as disclosed herein. In certain embodiments, the encoded AAV8 capsid has the sequence of SEQ ID NO: 143 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions, particularly substitutions with amino acid residues found in the corresponding position in other AAV capsids, for example, in the SUBS row of FIG. 21 which provides a comparison of the amino acid sequences of the capsid sequences of various AAVs, highlighting amino acids appropriate for substitution at different positions within the capsid sequence.

In specific embodiments, provided are constructs for gene therapy administration to a human subject, comprising an AAV vector, which comprises a viral capsid that is at least 95% identical to the amino acid sequence of an AAV2.7m8 capsid (SEQ ID NO: 142); and a viral or artificial genome comprising an expression cassette flanked by AAV inverted terminal repeats (ITRs) wherein the expression cassette comprises a transgene encoding the heavy and light chains of the therapeutic antibody, operably linked to one or more regulatory sequences that control expression of the transgene in human cells (e.g., retina cell or liver cell types) that express and deliver the therapeutic antibody in a therapeutically appropriate manner as disclosed herein. In certain embodiments, the encoded AAV2.7m8 capsid has the sequence of SEQ ID NO: 142 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions, particularly substitutions with amino acid residues found in the corresponding position in other AAV capsids, for example, in the SUBS row of FIG. 21 which provides a comparison of the amino acid sequences of the capsid sequences of various AAVs, highlighting amino acids appropriate for substitution at different positions within the capsid sequence.

In some embodiments, the HuPTM mAb or antigen binding fragment thereof, including the HuPTM Fab transgene should be controlled by appropriate expression control elements for expression of the HuPTM Fab or HuPTM Mab in human retina or liver cell types, for example, CB7 promoter (a chicken β-actin promoter and CMV enhancer, SEQ ID NO: 411), or tissue-specific promoters such as RPE-specific promoters e.g., the RPE65 promoter, or cone-specific promoters, e.g., the opsin promoter, or liver specific promoters, such as, the TBG (Thyroxine-binding Globulin) promoter (SEQ ID NO: 423), the Aβ0A2 promoter, the SERPINA1 (hAAT) promoter, the ApoE.hAAT promoter (SEQ ID NO: 412), or the MIR122 promoter, or inducible promoters, for example, hypoxia-induced promoters and drug inducible promoters such as promoters induced by rapamycin and related agents; and can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal). See, e.g., Powell and Rivera-Soto, 2015, Discov. Med., 19(102):49-57. In some embodiments, transgene expression is controlled by tissue-specific promoters or regulatory elements that promote tissue-specificity, for example, for liver-specific expression, LSPX1 (SEQ ID NO: 315), LSPX2 (SEQ ID NO: 316), LTP1 (SEQ ID NO: 317), LTP2 (SEQ ID NO: 318), or LTP3 (SEQ ID NO: 319), for liver and muscle expression, LMTP6 (SEQ ID NO: 320), LMTP13 (SEQ ID NO: 321), LMTP14 (SEQ ID NO: 322), LMTP15 (SEQ ID NO: 323), LMTP18 (SEQ ID NO: 324), LMTP19 (SEQ ID NO: 325), or LMTP20 (SEQ ID NO: 326), or for liver and bone expression, LBTP1 (SEQ ID NO: 327) or LBTP2 (SEQ ID NO: 328), the sequences of which are provided in Table 1.

Gene therapy constructs for antibodies or Fabs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. The leader sequence for each of the heavy and light chains is, e.g., MYRMQLLLLIALSLALVTNS (SEQ ID NO: 146). Section 5.1.5, supra, provides specific IRES, 2A, and other linker sequences that can be used with the methods and compositions provided herein. In specific embodiments, the linker is a Furin-2A linker, for example a Furin-F2A linker RKRR(GSG)APVKQTLNFDLLKLAGDVESNPGP (SEQ ID NO: 231) or a Furin-T2A linker RKRR(GSG)EGRGSLLTCGDVEENPGP (SEQ ID NO: 429). In specific embodiments, the transgene is a nucleotide sequence that encodes the following: Signal sequence-heavy chain Fab portion-Furin-(F/T)2A linker-signal sequence-light chain Fab portion. See FIGS. 2A-C for sequences for solanezumab, GSK933776, and lecanemab Fab expression, respectively; FIGS. 9A to 9C for sequences for sevacizumab and LKA-651 Fab expression, respectively; FIGS. 10A to 10D for a sequence for ascrinvacumab, tesidolumab, ravulizumab, and carotuximab Fab expression, respectively; FIG. 11 for sequences for ANX-007 Fab expression; FIGS. 12A to 12C for a sequence for adalimumab, infliximab, and golimumab Fab expression, respectively; FIGS. 16A to 16I for sequences for satralizumab, sarilumab, siltuximab, clazakizumab, sirukumab, olokizumab, gerilimzumab, and tocilizumab, inebilizumab Fab expression, respectively; and FIG. 19 for lanadelumab Fab expression (and see also Table 8 for nucleotide sequences encoding lanadelumab and lanadelumab transgenes). In an alternative embodiment, the construct also comprises the nucleotide sequence encoding the Fc domain for the therapeutic antibody (see Table 7 or FIG. 23) for expression of the full length mAb (and Table 8 for lanadelumab).

In a specific embodiment, the constructs described herein comprise the following components: (1) AAV2 inverted terminal repeats that flank the expression cassette; (2) Control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of the VEGF-binding, EpoR-binding, anti-Aβ, ALK-1-binding, C5-binding, ENG-binding, CC1Q-binding, TNFα-binding, kallikrein-binding, IL6R-binding, IL6-binding, and CD19-binding Fab, separated by a self-cleaving furin (F)/2A linker, ensuring expression of equal amounts of the heavy and the light chain polypeptides. An exemplary construct is provided in FIG. 1.

In another embodiment, the constructs described herein comprise the following components: (1) AAV2 inverted terminal repeats that flank the expression cassette; (2) Control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of the VEGF-binding, EpoR-binding, Aβ-binding, ALK-1-binding, C5-binding, ENG-binding, CC1Q-binding, TNFα-binding, kallikrein-binding, IL6R-binding, IL6-binding, and CD19-binding Fab, separated by flexible peptide linker, ensuring proper folding and solubility.

In specific embodiments, provided are AAV vectors comprising a viral capsid that is at least 95% identical to the amino acid sequence of an AAV8 capsid (SEQ ID NO: 143) or of an AAV2.7m8 (SEQ ID NO: 142); and an artificial genome comprising an expression cassette flanked by AAV inverted terminal repeats (ITRs), wherein the expression cassette comprises a transgene encoding an anti-VEGF, anti-EpoR, anti-Aβ, anti-ALK-1, anti-C5, anti-ENG, anti-CC1Q, anti-TNFα, anti-kallikrein, anti-IL6R, anti-IL6, and anti-CD19 mAb, or an antigen-binding fragment thereof, operably linked to one or more regulatory sequences that control expression of the transgene in one or more retina cell types (such as human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells).

### 5.5 Dose Administration

### 5.5.1Administration for Delivering to CNS

Sections 5.3.1, 5.3.2, 5.3.3, 5.3.4, 5.3.5., 5.3.6., and 5.3.7. describe recombinant vectors that contain a transgene encoding a HuPTM mAb or HuPTM Fab (or other antigen binding fragment of the HuPTM mAb) that binds to Aβ, sortilin, Tau protein, SEMA4D, alpha-synuclein, SOD1, and CGRPR, respectively. Therapeutically effective doses of any such recombinant vector should be administered in any manner such that the recombinant vector enters the CNS, e.g. by introducing the recombinant vector into the cerebral spinal fluid (CSF). In specific, embodiments, the vector is administered intrathecally, specifically intracisternally (such as to the cisterna magna) or, alternatively, lumbar delivery. Alternatively, the recombinant vector may be administered intravenously. In particular, recombinant AAV9 vectors have been shown to cross the blood-brain barrier and, as such, may be useful to deliver the anti-An, anti-sortilin, anti-Tau, anti-SEMA4D, anti-alpha-synuclein, anti-SOD1, or anti-CGRPR, antibody transgene product to the CNS. Specifically, a scAAV9 may be particularly useful for intravenous administration. Intrathecal, including intracisternal or lumbar administration, or intravenous administration should result expression of the soluble transgene product in cells of the CNS. The expression of the transgene product (e.g., the encoded anti-Aβ, anti-sortilin, anti-Tau, anti-SEMA4D, anti-alpha-synuclein, anti-SOD1, or anti-CGRPR antibody) results in delivery and maintenance of the transgene product in the CNS. Because the transgene product is continuously produced, maintenance of lower concentrations can be effective. The concentration of the transgene product can be measured in patient samples of the CSF.

Pharmaceutical compositions suitable for intrathecal, intracisternal, lumbar or intravenous administration comprise a suspension of the recombinant vector comprising the transgene encoding the anti-Aβ, anti-sortilin, anti-Tau, anti-SEMA4D, anti-alpha-synuclein, anti-SOD1, or anti-CGRPR antibody, or antigen-binding fragment thereof, in a formulation buffer comprising a physiologically compatible aqueous buffer. The formulation buffer can comprise one or more of a polysaccharide, a surfactant, polymer, or oil.

### 5.5.2 Administration for Delivering to Liver or Muscle Tissue

Sections 5.3.8, 5.3.9, 5.3.10, 5.3.11, 5.3.12, 5.3.13, 5.3.14, 5.3.15, 5.3.16, 5.3.17, 5.3.18, 5.3.19, 5.3.20, and 5.3.21, describe recombinant vectors that contain a transgene encoding a HuPTM mAb or HuPTM Fab (or other antigen binding fragment of the HuPTM mAb) that binds to VEGF, EpoR, Aβ, ALK-1, C5, ENG, CC1Q, TNFα, RGMa, TTR, CTGF, IL6R, IL6, CD19, ITGF7, SOST, pKal, IL/ILR, IgE, or TSLP. Therapeutically effective doses of any such recombinant vector should be administered in any manner such that the recombinant vector enters the liver or muscle (e.g., skeletal muscle), e.g. by introducing the recombinant vector into the bloodstream. Alternatively, the vector may be administered directly to the liver through hepatic blood flow, e.g., via the suprahepatic veins or via the hepatic artery. In specific, embodiments, the vector is administered subcutaneously, intramuscularly or intravenously. Intramuscular, subcutaneous, intravenous or hepatic administration should result in expression of the soluble transgene product in cells of the liver or muscle. Alternatively, the vector may be administered directly to the liver through hepatic blood flow, e.g., via the suprahepatic veins or via the hepatic artery. The expression of the transgene product (e.g., the encoded anti-VEGF, anti-EpoR, anti-Aβ, anti-ALK1, anti-C5, anti-ENG, anti-CC1Q, anti-TNFα, anti-RGMa, anti-TTR, anti-CTGF, anti-IL6R, anti-CD19, anti-ITGF7, anti-SOST, anti-pKal, anti-IL/ILR, anti-IgE, or anti-TSLP antibody) results in delivery and maintenance of the transgene product in the liver or muscle.

In specific embodiments, doses that maintain a plasma concentration of the anti-TNFα antibody transgene product at a Cₘᵢₙ of at least 0.5 μg/mL or at least 1 μg/mL (e.g., Cₘᵢₙ of 1 to 10 μg/ml, 3 to 30 μg/ml or 5 to 15 μg/mL or 5 to 30 μg/mL) are provided.

In specific embodiments, doses that maintain a plasma concentration of the adalimumab antibody, or antigen-binding fragment thereof, at a Cₘᵢₙ of at least 5 μg/mL (e.g., Cₘᵢₙ of 5 to 10 μg/ml or 10 to 20 μg/ml), preferably a Cₘᵢₙ of about 8 μg/mL to 9 μg/mL are provided.

In specific embodiments, doses that maintain a plasma concentration of the infliximab antibody, or antigen-binding fragment thereof, at a Cₘᵢₙ of at least 2 μg/mL (e.g., Cₘᵢₙ of 2 to 10 μg/ml or 10 to 20 μg/ml), preferably at a Cₘᵢₙ of about 5 μg/mL to 6 μg/mL, are provided.

In specific embodiments, doses that maintain a plasma concentration of the anti-CD19 transgene product at a Cₘᵢₙ of at least 1 μg/ml (e.g., Cₘᵢₙ of 1 to 10 μg/ml or 10 to 100 μg/ml or 100 to 300 μg/ml) are provided.

In specific embodiments, doses that maintain a plasma concentration of the anti-SOST transgene product at a Cₘᵢₙ of at least 1 mg/ml (e.g., C. of 1 to 10 μg/ml or 10 to 100 μg/ml or 100 to 200 μg/ml) are desired.

In specific embodiments, doses that maintain a plasma concentration of the sarilumab antibody, or antigen-binding fragment thereof, at a Cₘᵢₙ of at least 5 μg/ml (e.g., C. of 5 to 20 μg/ml or 20 to 50 μg/ml), preferably at a Cₘᵢₙ of about 15 μg/mL to 20 μg/mL, are provided.

In specific embodiments, doses that maintain a plasma concentration of the tocilizumab antibody, or antigen-binding fragment thereof, at a Cₘᵢₙ of at least 1 μg/ml (e.g., Cₘᵢₙ of 1 to 10 μg/ml or 10 to 20 μg/ml) are provided.

In specific embodiments, doses that maintain a plasma concentration of the siltuximab antibody, or an antigen-binding fragment thereof, at a Cₘᵢₙ of at least 20 μg/ml (e.g., Cₘᵢₙ of 20 to 100 μg/ml or 100 to 200 μg/ml), preferably at a Cₘᵢₙ of about 80 μg/mL to 90 μg/mL, are provided.

In specific embodiments, doses that maintain a plasma concentration of the anti-CTGF antibody transgene product at a Cₘᵢₙ of at least 100 μg/mL (e.g., Cₘᵢₙ of 100 to 200 μg/ml or 200 to 300 μg/mL) are provided.

In specific embodiments, doses that maintain a plasma concentration of the anti-ENG antibody transgene product at a Cₘᵢₙ of at least 10 μg/mL are desired, such as Cₘᵢₙ of 10 to 100 μg/ml or 100 to 300 μg/ml or 300 to 600 μg/ml are provided.

In specific embodiments, doses that maintain a plasma concentration of the anti-C5 antibody transgene product at a Cₘᵢₙ of at least 10 μg/mL (e.g., Cₘᵢₙ of 10 to 100 μg/ml or 100 to 200 mg/mL or 200 to 300 mg/mL) are provided.

In specific embodiments, doses that maintain a plasma concentration of the ravulizumab antibody, or antigen-binding fragment thereof, at a Cₘᵢₙ of at least 200 μg/mL (e.g., Cₘᵢₙ of 200 to 300 μg/ml or 300 to 400 μg/mL or 400 to 600 μg/mL), preferably at a Cₘᵢₙ of about 350 to 450 μg/mL in complement-inhibitor naïve patients and at a Cₘᵢₙ of about 450 to 550 μg/mL are provided.

In specific embodiments, doses that maintain a plasma concentration of the anti-IL/ILR antibody transgene product at a Cₘᵢₙ of at least 0.1 μg/mL (e.g., C. of 0.1 to 10 μg/ml or 10 to 20 μg/mL or 20 to 100 μg/mL) are provided.

In specific embodiments, doses that maintain a plasma concentration of the anti-IgE antibody transgene product at a Cₘᵢₙ of at least 100 μg/mL (e.g., Cₘᵢₙ of 100 to 200 μg/ml or 200 to 300 mg/mL or 300 to 400 μg/mL) are provided.

In specific embodiments, doses that maintain a plasma concentration of the anti-TSLP antibody transgene product at a Cₘᵢₙ of at least 70 μg/mL (e.g., Cₘᵢₙ of 70 to 150 μg/ml or 100 to 200 mg/mL or 200 to 350 mg/mL) are provided.

In specific embodiments, doses that maintain a plasma concentration of the lanadelumab antibody, or antigen-binding fragment thereof, at a Cₘᵢₙ of at least 10 μg/mL (e.g., Cₘᵢₙ of 10 to 50 μg/ml or 50 to 100 μg/mL or 100 to 200 μg/mL), preferably at a Cₘᵢₙ of about 20 to 30 μg/mL, are provided.

However, in all cases because the transgene product is continuously produced, maintenance of lower concentrations can be effective. Notwithstanding, because the transgene product is continuously produced, maintenance of lower concentrations can be effective. The concentration of the transgene product can be measured in patient blood serum samples.

Pharmaceutical compositions suitable for intravenous, intramuscular, subcutaneous or hepatic administration comprise a suspension of the recombinant vector comprising the transgene encoding the anti-VEGF, anti-EpoR, anti-Aβ, anti-ALK-1, anti-C5, anti-ENG, anti-CC1Q, anti-TNFα, anti-RGMa, anti-TTR, anti-CTGF, anti-IL6R, anti-IL6, anti-CD19, anti-ITGF7, anti-SOST, anti-pKal, anti-IL/ILR, anti-IgE, or anti-TSLP antibody, or antigen-binding fragment thereof, in a formulation buffer comprising a physiologically compatible aqueous buffer. The formulation buffer can comprise one or more of a polysaccharide, a surfactant, polymer, or oil.

### 5.5.3 Administration for Delivering to Retinal Type Cells

Therapeutically effective doses of the recombinant vector should be administered in any manner such that the recombinant vector enters the retina, e.g., by introducing the recombinant vector directly into the eye. In specific, embodiments, the vector is administered subretinally (a surgical procedure performed by trained retinal surgeons that involves a partial vitrectomy with the subject under local anesthesia, and injection of the gene therapy into the retina; see, e.g., Campochiaro et al., 2016, Hum Gen Ther September 26 epub:doi: 10.1089/hum.2016.117, which is incorporated by reference herein in its entirety), or intravitreally, or suprachoroidally such as by microinjection or microcannulation. (See, e.g., Patel et al., 2012, Invest Ophth & Vis Sci 53:4433-4441; Patel et al., 2011, Pharm Res 28:166-176; Olsen, 2006, Am J Ophth 142:777-787 each of which is incorporated by reference in its entirety). Subretinal, intravitreal or suprachoroidal administration should result in expression of the soluble transgene product in one or more of the following retinal cell types: human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells.

The expression of the transgene product (e.g., the encoded anti-VEGF, anti-EpoR, anti-anti-pKal, anti-ALK-1, anti-C5, anti-ENG, anti-CC1Q, anti-TNFα, anti-IL6R, anti-IL6, and anti-CD19 Antibody) results in delivery and maintenance of the transgene product in the retina. Pharmaceutical compositions suitable for administration comprise a suspension of the recombinant vector comprising the transgene encoding the anti-VEGF, anti-EpoR, anti-Aβ, anti-pKal, anti-ALK-1, anti-C5, anti-ENG, anti-CC1Q, anti-TNFα, anti-IL6R, anti-IL6, and anti-CD19 antibody, or antigen-binding fragment thereof, in a formulation buffer comprising a physiologically compatible aqueous buffer. The formulation buffer can comprise one or more of a polysaccharide, a surfactant, polymer, or

In specific embodiments, doses that maintain a plasma concentration of the adalimumab antibody, or antigen-binding fragment thereof, at a Cₘᵢₙ of at least 5 μg/mL (e.g., Cₘᵢₙ of 5 to 10 μg/ml or 10 to 20 μg/ml), preferably a Cₘᵢₙ of about 8 μg/mL to 9 μg/mL are provided. In specific embodiments, doses and routes of administration of a vector comprising the transgene encoding an adalimumab antibody, or antigen-binding fragment thereof, should result in expression of the adalimumab antibody, or antigen-binding fragment thereof, that achieve and maintain an intravitreal concentration of the adalimumab antibody, or antigen-binding fragment thereof, at an equivalent level to the intravitreal concentration achieved by monthly intravitreal injections of Humira at a dose of 1.5 mg.

In specific embodiments, doses that maintain a plasma concentration of the infliximab antibody, or antigen-binding fragment thereof, at a Cₘᵢₙ of at least 2 μg/mL (e.g., Cₘᵢₙ of 2 to 10 mg/ml or 10 to 20 μg/ml), preferably at a Cₘᵢₙ of about 5 μg/mL to 6 μg/mL, are provided.

In specific embodiments, doses that maintain a plasma concentration of the sarilumab antibody, or antigen-binding fragment thereof, at a Cₘᵢₙ of at least 5 mg/ml (e.g., C. of 5 to 20 mg/ml or 20 to 50 μg/ml), preferably at a Cₘᵢₙ of about 15 μg/mL to 20 μg/mL, are provided.

In specific embodiments, doses and routes of administration of a vector comprising the transgene encoding an satralizumab antibody, or antigen-binding fragment thereof, should result in expression of the satralizumab antibody, or antigen-binding fragment thereof, that achieve and maintain an intravitreal concentration of the satralizumab antibody, or antigen-binding fragment thereof, at an equivalent level to the intravitreal concentration achieved by monthly subcutaneous injections of SA237 (Roche) at a dose of 120 mg.

In specific embodiments, doses that maintain a plasma concentration of the tocilizumab antibody, or antigen-binding fragment thereof, at a Cₘᵢₙ of at least 1 μg/ml (e.g., C. of 1 to 10 μg/ml or 10 to 20 μg/ml) are provided. In specific embodiments, doses and routes of administration of a vector comprising the transgene encoding a tocilizumab antibody, or antigen-binding fragment thereof, should result in expression of the tocilizumab antibody, or antigen-binding fragment thereof, that achieve and maintain a systemic concentration of the adalimumab antibody, or antigen-binding fragment thereof, at an equivalent level to the systemic concentration achieved by monthly intravenous injections of RoActemra at a dose of 4 mg/kg or 8 mg/kg.

In specific embodiments, doses that maintain a plasma concentration of the siltuximab antibody, or an antigen-binding fragment thereof, at a Cm, of at least 20 μg/ml (e.g., Cₘᵢₙ of 20 to 100 μg/ml or 100 to 200 μg/ml), preferably at a Cₘᵢₙ of about 80 μg/mL to 90 μg/mL, are provided.

In specific embodiments, doses that maintain a plasma concentration of the ravulizumab antibody, or antigen-binding fragment thereof, at a Cₘᵢₙ of at least 200 μg/mL (e.g., Cₘᵢₙ of 200 to 300 μg/ml or 300 to 400 μg/mL or 400 to 600 μg/mL), preferably at a Cₘᵢₙ of about 350 to 450 μg/mL in complement-inhibitor naïve patients and at a Cₘᵢₙ of about 450 to 550 μg/mL are provided.

In specific embodiments, doses that maintain a plasma concentration of the lanadelumab antibody, or antigen-binding fragment thereof, at a Cₘᵢₙ of at least 10 μg/mL (e.g., Cₘᵢₙ Of 10 to 50 μg/ml or 50 to 100 μg/mL or 100 to 200 μg/mL), preferably at a Cₘᵢₙ of about 20 to 30 μg/mL, are provided.

### 5.6 Constructs and Methods for Expression of HuPTM mAb

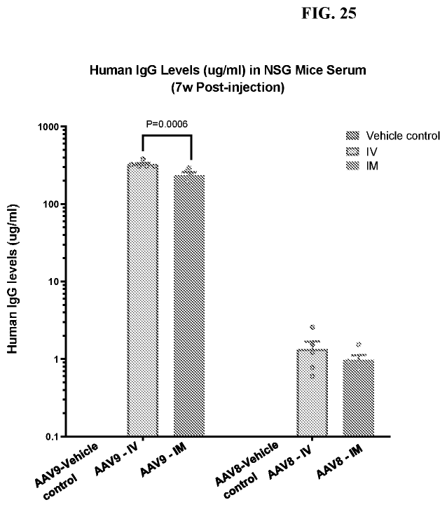

Examples 36 and 37 demonstrate that full length heavy and light chains of lanadelumab were expressed from an AAV vector transgene in human HEK293 cells and secreted into the cell supernatant (FIGS. 24B-24D). Mice administered recombinant AAV8 and recombinant AAV9 vectors that carry a transgene encoding the full length heavy and light chains of lanadelumab had lanadelumab present in serum as detectable by ELISA seven weeks after administration (FIG. 25). Accordingly, provided herein are AAV constructs, vectors, methods of manufacture and methods of treatment comprising transgenes that encode a full length heavy chain (including the heavy chain variable domain, the heavy chain constant domain 1 (C_{H}1), the hinge and Fc domain) and a full length light chain (light chain variable domain and light chain constant domain) that upon expression associate to form antigen-binding antibodies with Fc domains. The recombinant AAV constructs express the intact (i.e., full length) or substantially intact HuPTM mAb in a cell, cell culture, or in a subject. (“Substantially intact” refers to mAb having a sequence that is at least 95% identical to the full-length mAb sequence.) The nucleotide sequences encoding the heavy and light chains may be codon optimized for expression in human cells and have reduced incidence of CpG dimers in the sequence to promote expression in human cells. The transgenes may encode any full-length antibody. In preferred embodiments, the transgenes encode a full-length form of any of the therapeutic antibodies disclosed herein, for example, the Fab fragments of which depicted in FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F herein and including, in certain embodiments, the associated Fc domain provided in Table 7. In other embodiments, the transgenes encode the full-length forms of the Fab fragments of therapeutic antibodies depicted in one of FIGS. 2A-2F, 3A-3E, 4A, 4B, 5A-5C, 6, 7A, 7B, 8A-8H, 9A or 9B of PCT application no. PCT/US2018/056346, filed Oct. 17, 2018, incorporated herein by reference (which Figures are included as an Appendix hereto), with an Fc domain.

The full length mAb encoded by the transgene described herein preferably have the Fc domain of the full-length therapeutic antibody or is an Fc domain of the same type of immunoglobulin as the therapeutic antibody to be expressed. In certain embodiments, the Fc region is an IgG Fc region, but in other embodiments, the Fc region may be an IgA, IgD, IgE, or IgM. The Fc domain is preferably of the same isotype as the therapeutic antibody to be expressed, for example, if the therapeutic antibody is an IgG1 isotype, then the antibody expressed by the transgene comprises an IgG1 Fc domain. The antibody expressed from the transgene may have an IgG1, IgG2, IgG3 or IgG4 Fc domain.

The Fc region of the intact mAb has one or more effector functions that vary with the antibody isotype. The effector functions can be the same as that of the wild-type or the therapeutic antibody or can be modified therefrom to add, enhance, modify, or inhibit one or more effector functions using the Fc modifications disclosed in Section 5.1.9, infra. In certain embodiments, the HuPTM mAb transgene encodes a mAb comprising an Fc polypeptide comprising an amino acid sequence that is at least 85%, 86%, 87%, 88%, 89%, 90%, 91%, 92%, 93%, 94%, 95%, 96%, 97%, 98% or 99% identical to the sequence set forth in the Fc domain polypeptides of the therapeutic antibodies described herein as set forth in Table 7 or an exemplary Fc domain of an IgG1, IgG2 or IgG4 isotype as set forth in FIG. 23. In specific embodiments, the HuPTM mAb comprises a Fc polypeptide comprising an amino acid sequence of an Fc polypeptide in Table 7 or FIG. 23 with 1, 2, 3, 4, 5, 6, 7, 8, 9, 10, 11, 12, 13, 14, 15 or more amino acid substitutions, insertions or deletions. In some embodiments, the HuPTM mAb comprises a Fc polypeptide of a sequence that is a variant of the Fc polypeptide sequence in Table 7 or FIG. 23 in that the sequence has been modified with one or more of the techniques described in Section 5.1.9, infra, to alter the Fc polypeptide's effector function.

In specific embodiments, provided are recombinant AAV constructs such as the construct shown in FIG. 1 or FIG. 24A, for gene therapy administration to a human subject in order to express an intact or substantially intact HuPTM mAb in the subject. Gene therapy constructs are designed such that both the heavy and light chains are expressed in tandem from the vector including the Fc domain polypeptide of the heavy chain. In certain embodiments, the transgene encodes a transgene with heavy and light chain Fab fragment polypeptides as shown in one of FIGS. 2A-2C, 3, 4A-4C, 5, 6A-6C, 7A-7B, 8A-8C, 9A-9C, 10A-10D, 11, 12A-12C, 13, 14A-14B, 15, 16A-16I, 17, 18, 19, and 29A-29F or FIGS. 2A-2F, 3A-3E, 4A, 4B, 5A-5C, 6, 7A, 7B, 8A-8H, 9A or 9B of PCT application no. PCT/US2018/056346, filed Oct. 17, 2018 (copies of these figures are included in an Appendix hereto and are incorporated herein by reference also), yet have a heavy chain that further comprises an Fc domain polypeptide C terminal to the hinge region of the heavy chain (including the corresponding Fc domain set forth in Table 7 or an IgG1, IgG2 or IgG4 Fc domain as in FIG. 23). In specific embodiments, the transgene is a nucleotide sequence that encodes the following: Signal sequence-heavy chain Fab portion (including hinge region)-heavy chain Fc polypeptide-Furin-2A linker-signal sequence-light chain Fab portion.

Provided are recombinant AAV vectors that comprise the constructs encoding the full-length antibodies. The rAAVs include but are not limited to AAV based vectors comprising capsid components from one or more of AAV1, AAV2, AAV3, AAV4, AAV5, AAV6, AAV7, AAV8, AAV9, AAV10, AAV11, AAVrh10 or AAVrh20. In preferred embodiments, AAV based vectors provided herein comprise capsids from one or more of AAV8, AAV9, AAV10, AAV11, AAVrh10 or AAVrh20 serotypes. The AAV serotype may be advantageously selected for tropism to a particular tissue type as detailed herein.

The rAAV vectors that encode and express the full-length therapeutic antibodies may be administered to treat or prevent or ameliorate symptoms of a disease or condition amenable to treatment, prevention or amelioration of symptoms with the therapeutic antibodies. Also provided are methods of expressing HuPTM mAbs in human cells using the rAAV vectors and constructs encoding them.

### 5.6.1. Full Length Expression of HuPTM mAb in CNS Cells

In a specific embodiments for expressing an intact or substantially intact mAb in retinal cell types, the constructs described herein comprise the following components: (1) AAV2 inverted terminal repeats that flank the expression cassette; (2) Control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy chain Fab of an anti-An mAb (e.g., solanezumab, lecanemab, and GSK933776), anti-sortilin mAb (e.g., AL-001), anti-Tau protein mAb (e.g., ABBV-8E12, UCB-0107, NI-105 (BIIB076), and aTAU), anti-SEMA4D mAb (e.g., VX15/2503), anti-alpha-synuclein mAb (e.g., prasinezumab, NI-202 (BIIB054) and MED-1341), anti-SOD1 mAb (e.g., NI-204), or anti-CGRPR mAb (e.g., eptinezumab, fremanezumab, and galcanezumab, the Fc polypeptide may be the Fc associated with the therapeutic antibody (Table 7) or of the same isotype as the native form of the therapeutic antibody, such as an IgG isotype amino acid sequence from FIG. 23; and the light chain Fab of an anti-Aβ mAb (e.g., solanezumab, lecanemab, and GSK933776), anti-sortilin mAb (e.g., AL-001), anti-Tau protein mAb (e.g., ABBV-8E12, UCB-0107, NI-105 (BIIB076), and aTAU), anti-SEMA4D mAb (e.g., VX15/2503), anti-alpha-synuclein mAb (e.g., prasinezumab, NI-202 (BIIB054) and MED-1341), anti-SOD1 mAb (e.g., NI-204), or anti-CGRPR mAb (e.g., eptinezumab, fremanezumab, and galcanezumab, wherein the heavy chain (Fab and Fc polypeptide) and the light chain are separated by a self-cleaving furin (F)/2A or T2A or flexible linker, ensuring expression of equal amounts of the heavy and the light chain polypeptides. An exemplary construct is provided in FIG. 1.

In specific embodiments, provided are AAV vectors comprising a viral capsid that is at least 95% identical to, or is identical to, the amino acid sequence of an AAV9 capsid (SEQ ID NO: 144) or AAVrh10 (SEQ ID NO: 145); and an artificial genome comprising an expression cassette flanked by AAV inverted terminal repeats (ITRs), wherein the expression cassette comprises a transgene encoding an intact or substantially intact anti-Aβ, anti-sortilin, anti-Tau protein, anti-SEMA4D, anti-alpha-synuclein, anti-SOD1, or anti-CGRPR mAb, operably linked to one or more regulatory sequences that control expression of the transgene in human CNS cells. Methods of producing HuPTM mAbs and methods of treatment, prevention or amelioration of symptoms of a disease or conditions amenable to treatment, prevention or amelioration of symptoms with the therapeutic antibody by administration of an rAAV encoding the HuPTM mAb or with the HuPTM mAb are also provided.

### 5.6.2 Full Length Expression of mAB in Liver or Muscle Cells

In a specific embodiments for expressing an intact or substantially intact mAb in muscle or liver cell types, the constructs described herein comprise the following components: (1) AAV2 inverted terminal repeats that flank the expression cassette; (2) Control elements, which include a) an inducible promoter, preferably a hypoxia-inducible promoter, b) a chicken β-actin intron and c) a rabbit β-globin poly A signal; and (3) nucleic acid sequences coding for the heavy chain Fab of an anti-VEGF (e.g., sevacizumab), anti-EpoR (e.g., LKA-651), anti-ALK1 (e.g., ascrinvacumab), anti-C5 (e.g., tesidolumab and ravulizumab), anti-endoglin (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, sarilumab, and tocilizumab), anti-IL6 (e.g. siltuximab, clazakizumab, sirukumab, olokizumab, and gerilimzumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-IL/ILR (e.g. benralizumab, reslizumab, tralokinumab, and nemolizumab), anti-IgE (e.g. omalizumab), or anti-TSLP (e.g. Tezepelumab); an Fc polypeptide associated with the therapeutic antibody (Table 7) or of the same isotype as the native form of the therapeutic antibody, such as an IgG isotype amino acid sequence from FIG. 23; and the light chain of an anti-VEGF (e.g., sevacizumab), anti-EpoR (e.g., LKA-651), anti-ALK1 (e.g., ascrinvacumab), anti-C5 (e.g., tesidolumab and ravulizumab), anti-CD105 or anti-ENG (e.g., carotuximab), anti-CC1Q (e.g., ANX-007), anti-TNFα (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, sarilumab, and tocilizumab), anti-IL6 (e.g. siltuximab, clazakizumab, sirukumab, olokizumab, and gerilimzumab), anti-CD19 (e.g., inebilizumab), anti-ITGF7 mAb (e.g., etrolizumab), anti-SOST mAb (e.g., romosozumab), anti-pKal mAb (e.g., lanadelumab), anti-IL/ILR (e.g. benralizumab, reslizumab, tralokinumab, and nemolizumab), anti-IgE (e.g. omalizumab), or anti-TSLP (e.g. Tezepelumab); wherein the heavy chain (Fab and Fc region) and the light chain are separated by a self-cleaving furin (F)/F2A or T2A or flexible linker, ensuring expression of equal amounts of the heavy and the light chain polypeptides. An exemplary construct is provided in FIG. 1.

In specific embodiments, provided are AAV vectors comprising a viral capsid that is at least 95% identical to the amino acid sequence of an AAV8 capsid (SEQ ID NO: 143); and an artificial genome comprising an expression cassette flanked by AAV inverted terminal repeats (ITRs), wherein the expression cassette comprises a transgene encoding an intact or substantially intact anti-VEGF, anti-EpoR, anti-ALK-1, anti-Aβ anti-C5, anti-ENG, anti-CC1Q, anti-TNFα, anti-RGMa, anti-TTR, anti-CTGF, anti-CD19, anti-ITGF7, anti-SOST, anti-pKal mAb, anti-IL6R, anti-IL6, anti-IL/ILR, anti-IgE, or anti-TSLP; operably linked to one or more regulatory sequences that control expression of the transgene in human liver or muscle cells.

In specific embodiments, provided are AAV vectors comprising a viral capsid that is at least 95% identical to the amino acid sequence of an AAV9 (SEQ ID NO: 144); and an artificial genome comprising an expression cassette flanked by AAV inverted terminal repeats (ITRs), wherein the expression cassette comprises a transgene encoding an intact or substantially intact anti-VEGF, anti-EpoR, anti-Aβ, anti-ALK-1, anti-C5, anti-ENG, anti-CC1Q, anti-TNFα, anti-RGMa, anti-TTR, anti-CTGF, anti-IL6R, anti-IL6, anti-CD19, anti-ITGF7, anti-SOST, anti-pKal mAb, anti-IL/ILR, anti-ITGA4, anti-IgE, or anti-TSLP, operably linked to one or more regulatory sequences that control expression of the transgene in human muscle cells.

### 5.6.3 Full Length Expression of mAb in Retinal Cell Types

In a specific embodiments for expressing an intact or substantially intact mAb in retinal cell types, the constructs described herein comprise the following components: (1) AAV2 inverted terminal repeats that flank the expression cassette; (2) Control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy chain Fab of an anti-VEGF (e.g., sevacizumab), anti-EpoR (e.g., LKA-651), anti-Aβ (e.g., solanezumab, lecanemab, and GSK933776) anti-ALK1 (e.g., ascrinvacumab), anti-C5 (e.g., tesidolumab, ravulizumab), anti-CD105 or anti-ENG (e.g., carotuximab), anti-CC1Q (e.g., ANX-007), anti-TNFα (e.g., adalimumab, infliximab, and golimumab), anti-IL6R (e.g., satralizumab, sarilumab, and tocilizumab), anti-IL6 (e.g. siltuximab, clazakizumab, sirukumab, olokizumab, and gerilimzumab), anti-CD19 (e.g., inebilizumab), an Fc polypeptide associated with the therapeutic antibody (Table 6) or of the same IgG isotype as the native form of the therapeutic antibody, such as an IgG isotype amino acid sequence from FIG. 23, and a light chain of an anti-VEGF (e.g., sevacizumab), anti-Aβ (e.g., solanezumab, lecanemab, and GSK933776), anti-EpoR (e.g., LKA-651), anti-ALK1 (e.g., ascrinvacumab), anti-C5 (e.g., tesidolumab and ravulizumab), anti-CD105 or anti-Endoglin (e.g., carotuximab), anti-CC1Q (e.g., ANX-007), anti-TNFα (e.g., adalimumab, infliximab, and golimumab), anti-IL6R (e.g., satralizumab, sarilumab, and tocilizumab), anti-CD19 (e.g., inebilizumab), and anti-IL6 (e.g. siltuximab, clazakizumab, sirukumab, olokizumab, and gerilimzumab), wherein the heavy chain (Fab and Fc polypeptide) and light chain are separated by a self-cleaving furin (F)/F2A or flexible linker, ensuring expression of equal amounts of the heavy and the light chain polypeptides. An exemplary construct is provided in FIG. 1.

In specific embodiments, provided are AAV vectors comprising a viral capsid that is at least 95% identical to the amino acid sequence of an AAV8 capsid (SEQ ID NO: 143) or of an AAV2.7m8 (SEQ ID NO: 142); and an artificial genome comprising an expression cassette flanked by AAV inverted terminal repeats (ITRs), wherein the expression cassette comprises a transgene encoding an intact or substantially intact anti-VEGF, anti-fD, anti-MMP9, anti-EpoR, anti-Aβ, anti-ALK-1, anti-C5, anti-ENG, anti-CC1Q, anti-TNFα, anti-IL6R, anti-IL6, or anti-CD19 mAb, operably linked to one or more regulatory sequences that control expression of the transgene in one or more retina cell types (such as human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells).

### 6. EXAMPLES

### 6.1 Example 1: Solanezumab Fab cDNA-Based Vector

A solanezumab Fab cDNA-based vector is constructed comprising a transgene comprising nucleotide sequences encoding the Fab portion of the heavy and light chain sequences of solanezumab (amino acid sequences being SEQ ID NOs. 1 and 2, respectively). The nucleotide sequence coding for the Fab portion of the heavy and light chain is codon optimized for expression in human CNS cells. Nucleotide sequences may be the nucleotide sequence of SEQ ID NOs. 71 and 72, respectively. The transgene also comprises nucleotide sequences that encodes a signal peptide, e.g., MYRMQLLLLIALSLALVTNS (SEQ ID NO: 146). The nucleotide sequences encoding the light chain ad heavy chain are separated by IRES elements or 2A cleavage sites (SEQ ID NOs: 227-230) to create a bicistronic vector. See FIG. 2A for amino acid sequence of a transgene product. The vector additionally includes a constitutive promoter, such as CB7 or an inducible promoter, such as a hypoxia-inducible promoter.

### 6.2 Example 2: GSK933776 Fab cDNA-Based Vector

A GSK933776 Fab cDNA-based vector is constructed comprising a transgene comprising nucleotide sequences encoding the Fab portion of the heavy and light chain sequences of GSK933776 (amino acid sequences being SEQ ID NOs. 3 and 4, respectively). The nucleotide sequence coding for the Fab portion of the heavy and light chain is codon optimized for expression in human CNS cells. Nucleotide sequences may be the nucleotide sequence of SEQ ID NOs. 73 and 74, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 2B for amino acid sequence of a transgene product. The vector additionally includes a constitutive promoter, such as CB7 or an inducible promoter, such as a hypoxia-inducible promoter.

### 6.3 Example 3: AL-001 Fab cDNA-Based Vector

An AL-001 Fab cDNA-based vector is constructed comprising a transgene comprising nucleotide sequences encoding the Fab portion of the heavy and light chain sequences of AL-001 (amino acid sequences being SEQ ID NOs. 5 and 6, respectively). The nucleotide sequence coding for the Fab portion of the heavy and light chain is codon optimized for expression in human CNS cells and may be the nucleotide sequence of SEQ ID NOs. 75 and 76, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 3 for amino acid sequence of a transgene product. The vector additionally includes a constitutive promoter, such as CB7 or an inducible promoter, such as a hypoxia-inducible promoter.

### 6.4 Example 4: ABBV-8E12 Fab cDNA-Based Vector

An ABBV-8E12 Fab cDNA-based vector is constructed comprising a transgene comprising nucleotide sequences encoding the Fab portion of the heavy and light chain sequences of ABBV-8E12 (amino acid sequences being SEQ ID NOs. 7 and 8, respectively). The nucleotide sequence coding for the Fab portion of the heavy and light chain is codon optimized for expression in human CNS cells and may be the nucleotide sequence of SEQ ID NOs: 77 and 78, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 4A for amino acid sequence of a transgene product. The vector additionally includes a constitutive promoter, such as CB7 or an inducible promoter, such as a hypoxia-inducible promoter.

### 6.5 Example 5: UCB-0107 Fab cDNA-Based Vector

An UCB-0107 Fab cDNA-based vector is constructed comprising a transgene comprising nucleotide sequences encoding the Fab portion of the heavy and light chain sequences of UCB-0107 (amino acid sequences being SEQ ID NOs. 9 and 10, respectively). The nucleotide sequence coding for the Fab portion of the heavy and light chain is codon optimized for expression in human CNS cells and may be the nucleotide sequence of SEQ ID NOs. 79 and 80, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 4B for amino acid sequence of a transgene product. The vector additionally includes a constitutive promoter, such as CB7 or an inducible promoter, such as a hypoxia-inducible promoter.

### 6.6 Example 6: NI-105 Fab cDNA-Based Vector

A NI-105 Fab cDNA-based vector is constructed comprising a transgene comprising nucleotide sequences encoding the Fab portion of the heavy and light chain sequences of NI-105 (amino acid sequences being SEQ ID NOs. 11 and 12, respectively). The nucleotide sequence coding for the Fab portion of the heavy and light chain is codon optimized for expression in human CNS cells and may be the nucleotide sequence of SEQ ID NOs. 81 and 82, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 4C for amino acid sequence of a transgene product. The vector additionally includes a constitutive promoter, such as CB7 or an inducible promoter, such as a hypoxia-inducible promoter.

### 6.7 Example 7: VX15/2503 Fab cDNA-Based Vector

A VX15/2503 Fab cDNA-based vector is constructed comprising a transgene comprising nucleotide sequences encoding the Fab portion of the heavy and light chain sequences of VX15/2503 (amino acid sequences being SEQ ID NOs. 13 and 14, respectively). The nucleotide sequence coding for the Fab portion of the heavy and light chain is codon optimized for expression in human CNS cells and may be the nucleotide sequence of SEQ ID NOs: 83 and 84, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 5 for amino acid sequence of a transgene product. The vector additionally includes a constitutive promoter, such as CB7 or an inducible promoter, such as a hypoxia-inducible promoter.

### 6.8 Example 8: Prasinezumab Fab cDNA-Based Vector

A prasinezumab Fab cDNA-based vector is constructed comprising a transgene comprising nucleotide sequences encoding the Fab portion of the heavy and light chain sequences of prasinezumab (amino acid sequences being SEQ ID NOs. 15 and 16, respectively). The nucleotide sequence coding for the Fab portion of the heavy and light chain is codon optimized for expression in human CNS cells and may be the nucleotide sequence of SEQ ID NOs. 85 and 86, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 6A for amino acid sequence of a transgene product. The vector additionally includes a constitutive promoter, such as CB7 or an inducible promoter, such as a hypoxia-inducible promoter.

### 6.9 Example 9: NI-202 Fab cDNA-Based Vector

A NI-202 Fab cDNA-based vector is constructed comprising a transgene comprising nucleotide sequences encoding the Fab portion of the heavy and light chain sequences of NI-202 (amino acid sequences being SEQ ID NOs. 17 and 18, respectively). The nucleotide sequence coding for the Fab portion of the heavy and light chain is codon optimized for expression in human CNS cells and may be the nucleotide sequence of SEQ ID NOS: 87 and 88, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 6B for amino acid sequence of a transgene product. The vector additionally includes a constitutive promoter, such as CB7 or an inducible promoter, such as a hypoxia-inducible promoter.

### 6.10 Example 10: MEDI-1341/TAK 341 Fab cDNA-Based Vector

A MEDI-1341/TAK 341 Fab cDNA-based vector is constructed comprising a transgene comprising nucleotide sequences encoding the Fab portion of the heavy and light chain sequences of MEDI-1341/TAK 341 (amino acid sequences being SEQ ID NOs. 19 and 20, respectively). The nucleotide sequence coding for the Fab portion of the heavy and light chain is codon optimized for expression in human CNS cells and may be the nucleotide sequence of SEQ ID NOS: 89 and 90, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID Nos: 227-230) to create a bicistronic vector. See FIG. 6C for amino acid sequence of a transgene product. The vector additionally includes a constitutive promoter, such as CB7 or an inducible promoter, such as a hypoxia-inducible promoter.

### 6.11 Example 11: NI-204.10D12 Fab cDNA-Based Vector

A NI-204.10D12 Fab cDNA-based vector is constructed comprising a transgene comprising nucleotide sequences encoding the Fab portion of the heavy and light chain sequences of NI-204.10D12 (amino acid sequences being SEQ ID NOs. 21 and 22, respectively). The nucleotide sequence coding for the Fab portion of the heavy and light chain is codon optimized for expression in human CNS cells and may be the nucleotide sequence of SEQ ID NOs: 91 and 92, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 7A for amino acid sequence of a transgene product. The vector additionally includes a constitutive promoter, such as CB7 or an inducible promoter, such as a hypoxia-inducible promoter.

### 6.12 Example 12: NI-204.12G7 Fab cDNA-Based Vector

A NI-204.12G7 Fab cDNA-based vector is constructed comprising a transgene comprising nucleotide sequences encoding the Fab portion of the heavy and light chain sequences of NI-204.12G7 (amino acid sequences being SEQ ID NOs. 23 and 24, respectively). The nucleotide sequence coding for the Fab portion of the heavy and light chain is codon optimized for expression in human CNS cells and may be the nucleotide sequence of SEQ ID NOs: 93 and 94, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 7B for amino acid sequence of a transgene product. The vector additionally includes a constitutive promoter, such as CB7 or an inducible promoter, such as a hypoxia-inducible promoter.

### 6.13 Example 13: Eptinezumab Fab cDNA-Based Vector

An eptinezumab Fab cDNA-based vector is constructed comprising a transgene comprising nucleotide sequences encoding the Fab portion of the heavy and light chain sequences of eptinezumab (amino acid sequences being SEQ ID NOs. 25 and 26, respectively). The nucleotide sequence coding for the Fab portion of the heavy and light chain is codon optimized for expression in human CNS cells and may be the nucleotide sequence of SEQ ID NOs: 95 and 96, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 8A for amino acid sequence of a transgene product. The vector additionally includes a constitutive promoter, such as CB7 or an inducible promoter, such as a hypoxia-inducible promoter.

### 6.14 Example 14: Fremanezumab Fab cDNA-Based Vector

A fremanezumab Fab cDNA-based vector is constructed comprising a transgene comprising nucleotide sequences encoding the Fab portion of the heavy and light chain sequences of fremanezumab (amino acid sequences being SEQ ID NOs. 27 and 28, respectively). The nucleotide sequence coding for the Fab portion of the heavy and light chain is codon optimized for expression in human CNS cells and may be the nucleotide sequence of SEQ ID NOS: 97 and 98, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 8B for amino acid sequence of a transgene product. The vector additionally includes a constitutive promoter, such as CB7 or an inducible promoter, such as a hypoxia-inducible promoter.

### 6.15 Example 15: Galcanezumab Fab cDNA-Based Vector

A galcanezumab Fab cDNA-based vector is constructed comprising a transgene comprising nucleotide sequences encoding the Fab portion of the heavy and light chain sequences of galcanezumab (amino acid sequences being SEQ ID NOs. 29 and 30, respectively). The nucleotide sequence coding for the Fab portion of the heavy and light chain is codon optimized for expression in human CNS cells and may be the nucleotide sequence of SEQ ID NOs. 99 and 100, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 8C for amino acid sequence of a transgene product. The vector additionally includes a constitutive promoter, such as CB7 or an inducible promoter, such as a hypoxia-inducible promoter.

### 6.16 Example 16: Sevacizumab Fab cDNA-Based Vector

A sevacizumab Fab cDNA-based vector is constructed comprising a transgene comprising nucleotide sequences encoding the Fab portion of the heavy and light chain sequences of sevacizumab (amino acid sequences being SEQ ID NOs. 31 and 32, respectively). The nucleotide sequence coding for the Fab portion of the heavy and light chain is codon optimized for expression in human CNS cells and may be the nucleotide sequence of SEQ ID NOs. 101 and 102, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 9A for amino acid sequence of a transgene product. The vector additionally includes a constitutive promoter, such as CB7 or an inducible promoter, such as a hypoxia-inducible promoter.

### 6.17 Example 17: LKA-651 (NVS2) Fab cDNA-Based Vector

A LKA-651 (NVS2) Fab cDNA-based vector is constructed comprising a transgene comprising nucleotide sequences encoding the Fab portion of the heavy and light chain sequences of LKA-651 (NVS2) (amino acid sequences being SEQ ID NOs. 33 and 34, respectively). The nucleotide sequence coding for the Fab portion of the heavy and light chain is codon optimized for expression in human CNS cells and may be the nucleotide sequence of SEQ ID NOs. 103 and 104, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 9B for amino acid sequence of a transgene product. The vector additionally includes a constitutive promoter, such as CB7 or an inducible promoter, such as a hypoxia-inducible promoter.

### 6.18 Example 18: LKA-651 (NVS3) Fab cDNA-Based Vector

A LKA-651 (NVS3) Fab cDNA-based vector is constructed comprising a transgene comprising nucleotide sequences encoding the Fab portion of the heavy and light chain sequences of LKA-651 (NVS3) (amino acid sequences being SEQ ID NOs. 35 and 36, respectively). The nucleotide sequence coding for the Fab portion of the heavy and light chain is codon optimized for expression in human CNS cells and may be the nucleotide sequence of SEQ ID NOs. 105 and 106, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 9C for amino acid sequence of a transgene product. The vector additionally includes a constitutive promoter, such as CB7 or an inducible promoter, such as a hypoxia-inducible promoter.

### 6.19 Example 19: Ascrinvacumab Fab cDNA-Based Vector

An ascrinvacumab Fab cDNA-based vector is constructed comprising a transgene comprising nucleotide sequences encoding the Fab portion of the heavy and light chain sequences of ascrinvacumab (amino acid sequences being SEQ ID NOs. 37 and 38, respectively). The nucleotide sequence coding for the Fab portion of the heavy and light chain is codon optimized for expression in human CNS cells and may be the nucleotide sequence of SEQ ID NOs. 107 and 108, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 10A for amino acid sequence of a transgene product. The vector additionally includes a constitutive promoter, such as CB7 or an inducible promoter, such as a hypoxia-inducible promoter.

### 6.20 Example 20: Tesidolumab Fab cDNA-Based Vector

A tesidolumab Fab cDNA-based vector is constructed comprising a transgene comprising nucleotide sequences encoding the Fab portion of the heavy and light chain sequences of tesidolumab (amino acid sequences being SEQ ID NOs. 39 and 40, respectively). The nucleotide sequence coding for the Fab portion of the heavy and light chain is codon optimized for expression in human CNS cells and may be the nucleotide sequence of SEQ ID NOs. 109 and 110, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 10B for amino acid sequence of a transgene product. The vector additionally includes a constitutive promoter, such as CB7 or an inducible promoter, such as a hypoxia-inducible promoter.

### 6.21 Example 21: Carotuximab Fab cDNA-Based Vector

A carotuximab Fab cDNA-based vector is constructed comprising a transgene comprising nucleotide sequences encoding the Fab portion of the heavy and light chain sequences of carotuximab (amino acid sequences being SEQ ID NOs. 41 and 42, respectively). The nucleotide sequence coding for the Fab portion of the heavy and light chain is codon optimized for expression in human CNS cells and may be the nucleotide sequence of SEQ ID NOS: 111 and 112, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 10C for amino acid sequence of a transgene product. The vector additionally includes a constitutive promoter, such as CB7 or an inducible promoter, such as a hypoxia-inducible promoter.

### 6.22 Example 22: ANX-007 Fab cDNA-Based Vector

An ANX-007 Fab cDNA-based vector is constructed comprising a transgene comprising nucleotide sequences encoding the Fab portion of the heavy and light chain sequences of ANX-007 (amino acid sequences being SEQ ID NOs. 43 and 44, respectively). The nucleotide sequence coding for the Fab portion of the heavy and light chain is codon optimized for expression in human CNS cells and may be the nucleotide sequence of SEQ ID NOs. 113 and 114, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 11 for amino acid sequence of a transgene product. The vector additionally includes a constitutive promoter, such as CB7 or an inducible promoter, such as a hypoxia-inducible promoter.

### 6.23 Example 23: Adalimumab Fab cDNA-Based Vector

An adalimumab Fab cDNA-based vector is constructed comprising a transgene comprising nucleotide sequences encoding the Fab portion of the heavy and light chain sequences of adalimumab (amino acid sequences being SEQ ID NOs. 45 and 46, respectively). The nucleotide sequence coding for the Fab portion of the heavy and light chain is codon optimized for expression in human CNS cells and may be the nucleotide sequence of SEQ ID NOs. 115 and 116, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 12A for amino acid sequence of a transgene product. The vector additionally includes a constitutive promoter, such as CB7 or an inducible promoter, such as a hypoxia-inducible promoter.

### 6.24 Example 24: Infliximab Fab cDNA-Based Vector

An infliximab Fab cDNA-based vector is constructed comprising a transgene comprising nucleotide sequences encoding the Fab portion of the heavy and light chain sequences of infliximab (amino acid sequences being SEQ ID NOs. 47 and 48, respectively). The nucleotide sequence coding for the Fab portion of the heavy and light chain is codon optimized for expression in human CNS cells and may be the nucleotide sequence of SEQ ID NOs. 117 and 118, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 12B for amino acid sequence of a transgene product. The vector additionally includes a constitutive promoter, such as CB7 or an inducible promoter, such as a hypoxia-inducible promoter.

### 6.25 Example 25: Golimumab Fab cDNA-Based Vector

A golimumab Fab cDNA-based vector is constructed comprising a transgene comprising nucleotide sequences encoding the Fab portion of the heavy and light chain sequences of golimumab (amino acid sequences being SEQ ID NOs. 49 and 50, respectively). The nucleotide sequence coding for the Fab portion of the heavy and light chain is codon optimized for expression in human CNS cells and may be the nucleotide sequence of SEQ ID NOs. 119 and 120, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 12C for amino acid sequence of a transgene product. The vector additionally includes a constitutive promoter, such as CB7 or an inducible promoter, such as a hypoxia-inducible promoter.

### 6.26 Example 26: Elezanumab Fab cDNA-Based Vector

An elezanumab Fab cDNA-based vector is constructed comprising a transgene comprising nucleotide sequences encoding the Fab portion of the heavy and light chain sequences of elezanumab (amino acid sequences being SEQ ID NOs. 51 and 52, respectively). The nucleotide sequence coding for the Fab portion of the heavy and light chain is codon optimized for expression in human CNS cells and may be the nucleotide sequence of SEQ ID NOS: 121 and 122, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 13 for amino acid sequence of a transgene product. The vector additionally includes a constitutive promoter, such as CB7 or an inducible promoter, such as a hypoxia-inducible promoter.

### 6.27 Example 27: NI-301 Fab cDNA-Based Vector

An NI-301 Fab cDNA-based vector is constructed comprising a transgene comprising nucleotide sequences encoding the Fab portion of the heavy and light chain sequences of NI-301 (amino acid sequences being SEQ ID NOs. 53 and 54, respectively). The nucleotide sequence coding for the Fab portion of the heavy and light chain is codon optimized for expression in human CNS cells and may be the nucleotide sequence of SEQ ID NOs. 123 and 124, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 14A for amino acid sequence of a transgene product. The vector additionally includes a constitutive promoter, such as CB7 or an inducible promoter, such as a hypoxia-inducible promoter.

### 6.28 Example 28: PRX-004 Fab cDNA-Based Vector

A PRX-004 Fab cDNA-based vector is constructed comprising a transgene comprising nucleotide sequences encoding the Fab portion of the heavy and light chain sequences of PRX-004 (amino acid sequences being SEQ ID NOs. 55 and 56, respectively). The nucleotide sequence coding for the Fab portion of the heavy and light chain is codon optimized for expression in human CNS cells and may be the nucleotide sequence of SEQ ID NOs. 125 and 126, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 14B for amino acid sequence of a transgene product. The vector additionally includes a constitutive promoter, such as CB7 or an inducible promoter, such as a hypoxia-inducible promoter.

### 6.29 Example 29: Pamrevlumab Fab cDNA-Based Vector

A pamrevlumab Fab cDNA-based vector is constructed comprising a transgene comprising nucleotide sequences encoding the Fab portion of the heavy and light chain sequences of pamrevlumab (amino acid sequences being SEQ ID NOs. 57 and 58, respectively). The nucleotide sequence coding for the Fab portion of the heavy and light chain is codon optimized for expression in human CNS cells and may be the nucleotide sequence of SEQ ID NOs. 127 and 128, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 15 for amino acid sequence of a transgene product. The vector additionally includes a constitutive promoter, such as CB7 or an inducible promoter, such as a hypoxia-inducible promoter.

### 6.30 Example 30: Satralizumab Fab cDNA-Based Vector

A satralizumab Fab cDNA-based vector is constructed comprising a transgene comprising nucleotide sequences encoding the Fab portion of the heavy and light chain sequences of satralizumab (amino acid sequences being SEQ ID NOs. 59 and 60, respectively). The nucleotide sequence coding for the Fab portion of the heavy and light chain is codon optimized for expression in human CNS cells and may be the nucleotide sequence of SEQ ID NOs. 129 and 130, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 16A for amino acid sequence of a transgene product. The vector additionally includes a constitutive promoter, such as CB7 or an inducible promoter, such as a hypoxia-inducible promoter.

### 6.31 Example 31: Sarilumab Fab cDNA-Based Vector

A sarilumab Fab cDNA-based vector is constructed comprising a transgene comprising nucleotide sequences encoding the Fab portion of the heavy and light chain sequences of sarilumab (amino acid sequences being SEQ ID NOs. 61 and 62, respectively). The nucleotide sequence coding for the Fab portion of the heavy and light chain is codon optimized for expression in human CNS cells and may be the nucleotide sequence of SEQ ID NOs. 131 and 132, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 16B for amino acid sequence of a transgene product. The vector additionally includes a constitutive promoter, such as CB7 or an inducible promoter, such as a hypoxia-inducible promoter.

### 6.32 Example 32: Inebilizumab Fab cDNA-Based Vector

An inebilizumab Fab cDNA-based vector is constructed comprising a transgene comprising nucleotide sequences encoding the Fab portion of the heavy and light chain sequences of inebilizumab (amino acid sequences being SEQ ID NOs. 63 and 64, respectively). The nucleotide sequence coding for the Fab portion of the heavy and light chain is codon optimized for expression in human CNS cells and may be the nucleotide sequence of SEQ ID NOs. 133 and 134, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 16C for amino acid sequence of a transgene product. The vector additionally includes a constitutive promoter, such as CB7 or an inducible promoter, such as a hypoxia-inducible promoter.

### 6.33 Example 33: Etrolizumab Fab cDNA-Based Vector

An etrolizumab Fab cDNA-based vector is constructed comprising a transgene comprising nucleotide sequences encoding the Fab portion of the heavy and light chain sequences of etrolizumab (amino acid sequences being SEQ ID NOs. 65 and 66, respectively). The nucleotide sequence coding for the Fab portion of the heavy and light chain is codon optimized for expression in human CNS cells and may be the nucleotide sequence of SEQ ID NOS: 135 and 136, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 17 for amino acid sequence of a transgene product. The vector additionally includes a constitutive promoter, such as CB7 or an inducible promoter, such as a hypoxia-inducible promoter.

### 6.34 Example 34: Romosozumab Fab cDNA-Based Vector

A romosozumab Fab cDNA-based vector is constructed comprising a transgene comprising nucleotide sequences encoding the Fab portion of the heavy and light chain sequences of romosozumab (amino acid sequences being SEQ ID NOs. 67 and 68, respectively). The nucleotide sequence coding for the Fab portion of the heavy and light chain may be the nucleotide sequence of SEQ ID NOS: 137 and 138, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 18 for amino acid sequence of a transgene product. The vector additionally includes a constitutive promoter, such as CB7 or an inducible promoter, such as a hypoxia-inducible promoter.

### 6.35 Example 35: Siltuximab Fab cDNA-Based Vector

A siltuximab Fab cDNA-based vector is constructed comprising a transgene comprising nucleotide sequences encoding the Fab portion of the heavy and light chain sequences of siltuximab (amino acid sequences being SEQ ID NOs. 331 and 332, respectively). The nucleotide sequence coding for the Fab portion of the heavy and light and may be the nucleotide sequence of SEQ ID NOs: 343 and 344, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 16C for amino acid sequence of a transgene product. The vector additionally includes a constitutive promoter, such as CB7 or an inducible promoter, such as a hypoxia-inducible promoter.

### 6.36 Example 36: Clazakizumab Fab cDNA-Based Vector

A clazakizumab Fab cDNA-based vector is constructed comprising a transgene comprising nucleotide sequences encoding the Fab portion of the heavy and light chain sequences of clazakizumab (amino acid sequences being SEQ ID NOs. 333 and 334, respectively). The nucleotide sequence coding for the Fab portion of the heavy and light chain may be the nucleotide sequence of SEQ ID NOs: 345 and 346, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 16D for amino acid sequence of a transgene product. The vector additionally includes a constitutive promoter, such as CB7 or an inducible promoter, such as a hypoxia-inducible promoter.

### 6.37 Example 37: Sirukumab Fab cDNA-Based Vector

A sirukumab Fab cDNA-based vector is constructed comprising a transgene comprising nucleotide sequences encoding the Fab portion of the heavy and light chain sequences of sirukumab (amino acid sequences being SEQ ID NOs. 335 and 336, respectively). The nucleotide sequence coding for the Fab portion of the heavy and light chain may be the nucleotide sequence of SEQ ID NOs: 347 and 348, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 16E for amino acid sequence of a transgene product. The vector additionally includes a constitutive promoter, such as CB7 or an inducible promoter, such as a hypoxia-inducible promoter.

### 6.38 Example 38: Olokizumab Fab cDNA-Based Vector

A olokizumab Fab cDNA-based vector is constructed comprising a transgene comprising nucleotide sequences encoding the Fab portion of the heavy and light chain sequences of olokizumab (amino acid sequences being SEQ ID NOs. 337 and 338, respectively). The nucleotide sequence coding for the Fab portion of the heavy and light chain may be the nucleotide sequence of SEQ ID NOs: 349 and 350, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 16F for amino acid sequence of a transgene product. The vector additionally includes a constitutive promoter, such as CB7 or an inducible promoter, such as a hypoxia-inducible promoter.

### 6.39 Example 39: Gerilimzumab Fab cDNA-Based Vector

A gerilimzumab Fab cDNA-based vector is constructed comprising a transgene comprising nucleotide sequences encoding the Fab portion of the heavy and light chain sequences of gerilimzumab (amino acid sequences being SEQ ID NOs. 339 and 340, respectively). The nucleotide sequence coding for the Fab portion of the heavy and light chain may be the nucleotide sequence of SEQ ID NOs: 351 and 352, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 16G for amino acid sequence of a transgene product. The vector additionally includes a constitutive promoter, such as CB7 or an inducible promoter, such as a hypoxia-inducible promoter.

### 6.40 Example 40: Tocilizumab Fab cDNA-Based Vector

A tocilizumab Fab cDNA-based vector is constructed comprising a transgene comprising nucleotide sequences encoding the Fab portion of the heavy and light chain sequences of tocilizumab (amino acid sequences being SEQ ID NOs. 341 and 342, respectively). The nucleotide sequence coding for the Fab portion of the heavy and light chain may be the nucleotide sequence of SEQ ID NOs: 353 and 354, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 16H for amino acid sequence of a transgene product. The vector additionally includes a constitutive promoter, such as CB7 or an inducible promoter, such as a hypoxia-inducible promoter.

### 6.41 Example 41: Lecanemab Fab cDNA-Based Vector

A lecanemab Fab cDNA-based vector is constructed comprising a transgene comprising nucleotide sequences encoding the Fab portion of the heavy and light chain sequences of lecanemab (amino acid sequences being SEQ ID NOs. 360 and 361, respectively). The nucleotide sequence coding for the Fab portion of the heavy and light chain may be the nucleotide sequence of SEQ ID NOs: 376 and 377, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 2C for amino acid sequence of a transgene product. The vector additionally includes a constitutive promoter, such as CB7 or an inducible promoter, such as a hypoxia-inducible promoter.

### 6.42 Example 42: Ravulizumab Fab cDNA-Based Vector

A ravulizumab Fab cDNA-based vector is constructed comprising a transgene comprising nucleotide sequences encoding the Fab portion of the heavy and light chain sequences of ravulizumab (amino acid sequences being SEQ ID NOs. 362 and 363, respectively). The nucleotide sequence coding for the Fab portion of the heavy and light chain may be the nucleotide sequence of SEQ ID NOs: 378 and 379, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 10D for amino acid sequence of a transgene product. The vector additionally includes a constitutive promoter, such as CB7 or an inducible promoter, such as a hypoxia-inducible promoter.

### 6.43 Example 43: Benralizumab Fab cDNA-Based Vector

A benralizumab Fab cDNA-based vector is constructed comprising a transgene comprising nucleotide sequences encoding the Fab portion of the heavy and light chain sequences of benralizumab (amino acid sequences being SEQ ID NOs. 364 and 365, respectively). The nucleotide sequence coding for the Fab portion of the heavy and light chain may be the nucleotide sequence of SEQ ID NOs: 380 and 381, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 29A for amino acid sequence of a transgene product. The vector additionally includes a constitutive promoter, such as CB7 or an inducible promoter, such as a hypoxia-inducible promoter.

### 6.44 Example 44: Reslizumab Fab cDNA-Based Vector

A reslizumab Fab cDNA-based vector is constructed comprising a transgene comprising nucleotide sequences encoding the Fab portion of the heavy and light chain sequences of reslizumab (amino acid sequences being SEQ ID NOs. 366 and 367, respectively). The nucleotide sequence coding for the Fab portion of the heavy and light chain may be the nucleotide sequence of SEQ ID NOs: 382 and 383, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 29B for amino acid sequence of a transgene product. The vector additionally includes a constitutive promoter, such as CB7 or an inducible promoter, such as a hypoxia-inducible promoter.

### 6.45 Example 45: Tralokinumab Fab cDNA-Based Vector

A tralokinumab Fab cDNA-based vector is constructed comprising a transgene comprising nucleotide sequences encoding the Fab portion of the heavy and light chain sequences of tralokinumab (amino acid sequences being SEQ ID NOs. 368 and 369, respectively). The nucleotide sequence coding for the Fab portion of the heavy and light chain may be the nucleotide sequence of SEQ ID NOs: 384 and 385, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 29C for amino acid sequence of a transgene product. The vector additionally includes a constitutive promoter, such as CB7 or an inducible promoter, such as a hypoxia-inducible promoter.

### 6.46 Example 46: Nemolizumab Fab cDNA-Based Vector

A nemolizumab Fab cDNA-based vector is constructed comprising a transgene comprising nucleotide sequences encoding the Fab portion of the heavy and light chain sequences of nemolizumab (amino acid sequences being SEQ ID NOs. 370 and 371, respectively). The nucleotide sequence coding for the Fab portion of the heavy and light chain may be the nucleotide sequence of SEQ ID NOs: 386 and 387, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 29D for amino acid sequence of a transgene product. The vector additionally includes a constitutive promoter, such as CB7 or an inducible promoter, such as a hypoxia-inducible promoter.

### 6.47 Example 47: Omalizumab Fab cDNA-Based Vector

A omalizumab Fab cDNA-based vector is constructed comprising a transgene comprising nucleotide sequences encoding the Fab portion of the heavy and light chain sequences of omalizumab (amino acid sequences being SEQ ID NOs. 372 and 373, respectively). The nucleotide sequence coding for the Fab portion of the heavy and light chain may be the nucleotide sequence of SEQ ID NOs: 388 and 389, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 29E for amino acid sequence of a transgene product. The vector additionally includes a constitutive promoter, such as CB7 or an inducible promoter, such as a hypoxia-inducible promoter.

### 6.48 Example 48: Tezepelumab Fab cDNA-Based Vector

A tezepelumab Fab cDNA-based vector is constructed comprising a transgene comprising nucleotide sequences encoding the Fab portion of the heavy and light chain sequences of tezepelumab (amino acid sequences being SEQ ID NOs. 374 and 375, respectively). The nucleotide sequence coding for the Fab portion of the heavy and light chain may be the nucleotide sequence of SEQ ID NOs: 390 and 391, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 29F for amino acid sequence of a transgene product. The vector additionally includes a constitutive promoter, such as CB7 or an inducible promoter, such as a hypoxia-inducible promoter.

### 6.49 Example 49: Lanadelumab Fab cDNA-Based Vector

A lanadelumab Fab cDNA-based vector is constructed comprising a transgene comprising nucleotide sequences encoding the Fab portion of the heavy and light chain sequences of lanadelumab (amino acid sequences being SEQ ID NOs. 69 and 70, respectively). The nucleotide sequence coding for the Fab portion of the heavy and light chain may be the nucleotide sequence of SEQ ID NOs. 139 and 140, respectively. The transgene also comprises nucleotide sequences that encodes a signal peptide, e.g., MYRMQLLLLIALSLALVTNS (SEQ ID NO: 146). The nucleotide sequences encoding the light chain and heavy chain are separated by IRES elements or 2A cleavage sites (SEQ ID NOs: 227-230) to create a bicistronic vector. See FIG. 19 for amino acid sequence of a transgene product. The vector additionally includes a constitutive promoter, such as CB7 or an inducible promoter, such as a hypoxia-inducible promoter.

### 6.50 Example 50: Protein Expression Analysis of Lanadelumab in Cell Lysates and Supernatant

Cell culture studies were performed to assess the expression of full length mAb sequences (containing Fc region) from AAV constructs in human cells.

### Methods

A lanadelumab cDNA-based vector was constructed comprising a transgene comprising a nucleotide sequence encoding the heavy and light chain sequences of lanadelumab (amino acid sequences being SEQ ID NOs. 69 and 70, respectively). The nucleotide sequence coding for the heavy and light chain of lanadelumab was codon optimized to generate the three nucleotide sequences provided in Table 8 below, L01 (SEQ ID NO: 141), L02 (SEQ ID NO: 286), and L03 (SEQ ID NO: 287). L02 and L03 also have reduced incidence of CpG dimers in the sequence. The transgene also comprised a nucleotide sequence that encodes the signal peptide MYRMQLLLLIALSLALVTNS (SEQ ID NO: 146). The nucleotide sequences encoding the light chain and heavy chain were separated by a Furin-F2A linker (SEQ ID NOs: 231) or a Furin T2A linker (SEQ ID NO: 429) to create a bicistronic vector. The vector additionally included a constitutive CAG promoter (SEQ ID NO: 411). See FIG. 24A for a schematic showing the genomic configuration and sequences of the constructs are provided in Table 8 (SEQ ID NOS: 435-437)

Table 1 above provides the sequences of composite nucleic acid regulatory sequences that may be incorporated into expression cassettes and be operably linked to the transgene to promote liver-specific expression (LSPX1, LSPX2, LTP1, LTP2, or LTP3, SEQ ID NOS: 315-319, respectively), liver and muscle expression (LMTP6, LMTP13, LMTP15, LMTP18, LMTP19 or LMTP20, SEQ ID NOS: 320-326 respectively), liver and bone expression (LTBP1 or LPTP2, SEQ ID NOS: 327-328, respectively) Other promoter sequences provided, include the ApoE.hAAT (SEQ ID NO: 412, Table 1 above) promoter, wherein four copies of the liver-specific apolipoprotein E (ApoE) enhancer were placed upstream of the human alpha 1-antitrypsin (hAAT) promoter).

TABLE 8

| Name/ | |
|---|---|
| SEQ ID NO. | Sequence |
| L01 | ATGTACCGGATGCAGCTGCTGCTGCTCATTGCCCTGTCTCTGGCCCTGGTCACCAATAGC |
| SEQ ID NO: 141 | GAAGTCCAGCTGCTGGAATCTGGCGGCGGACTTGTTCAACCTGGCGGCTCTCTGAGACTG |
| | AGCTGTGCCGCTTCCGGCTTCACCTTCAGCCACTATATCATGATGTGGGTCCGACAGGCC |
| | CCTGGCAAAGGCCTTGAATGGGTGTCCGGCATCTATAGCAGCGGCGGCATCACAGTGTAC |
| | GCCGACTCTGTGAAGGGCAGATTCACCATCAGCCGGGACAACAGCAAGAACACCCTGTAC |
| | CTGCAGATGAACAGCCTGAGAGCCGAGGACACCGCCGTGTACTACTGCGCCTATAGAAGA |
| | ATCGGCGTGCCCAGACGGGACGAGTTCGATATTTGGGGCCAGGGCACCATGGTCACCGTG |
| | TCTAGCGCCTCTACAAAGGGCCCTAGCGTGTTCCCTCTGGCTCCTAGCAGCAAGAGCACA |
| | AGCGGAGGAACAGCCGCTCTGGGCTGTCTGGTCAAGGACTACTTTCCCGAGCCTGTGACC |
| | GTGTCCTGGAATAGCGGAGCACTGACAAGCGGCGTGCACACCTTTCCAGCTGTGCTGCAA |
| | AGCAGCGGCCTGTACTCTCTGAGCAGCGTGGTCACAGTGCCAAGCTCTAGCCTGGGCACC |
| | CAGACCTACATCTGCAATGTGAACCACAAGCCTAGCAACACCAAGGTGGACAAGAGAGTG |
| | GAACCCAAGAGCTGCGACAAGACCCACACCTGTCCTCCATGTCCTGCTCCAGAACTGCTC |
| | GGCGGACCTTCCGTGTTCCTGTTTCCTCCAAAGCCTAAGGACACCCTGATGATCAGCAGA |
| | ACCCCTGAAGTGACCTGCGTGGTGGTGGATGTGTCCCACGAGGATCCCGAAGTGAAGTTC |
| | AATTGGTACGTGGACGGCGTGGAAGTGCACAACGCCAAGACCAAGCCTAGAGAGGAACAG |
| | TACAACAGCACCTACAGAGTGGTGTCCGTGCTGACCGTGCTGCACCAGGATTGGCTGAAC |
| | GGCAAAGAGTACAAGTGCAAGGTGTCCAACAAGGCCCTGCCTGCTCCTATCGAGAAAACC |
| | ATCAGCAAGGCCAAGGGCCAGCCTAGGGAACCCCAGGTTTACACACTGCCTCCAAGCCGG |
| | GAAGAGATGACCAAGAACCAGGTGTCCCTGACCTGCCTCGTGAAGGGCTTCTACCCTTCC |
| | GATATCGCCGTGGAATGGGAGAGCAATGGCCAGCCAGAGAACAACTACAAGACAACCCCT |
| | CCTGTGCTGGACAGCGACGGCTCATTCTTCCTGTACAGCAAGCTGACAGTGGACAAGTCC |
| | AGATGGCAGCAGGGCAACGTGTTCAGCTGCAGCGTGATGCACGAGGCCCTGCACAACCAC |
| | TACACCCAGAAGAGTCTGAGCCTGTCTCCAGGCCTGCGGAAGAGAAGGGCTCCTGTGAAG |
| | CAGACCCTGAACTTCGACCTGCTGAAACTGGCCGGCGACGTGGAAAGCAACCCCGGACCT |
| | ATGTATAGAATGCAGCTCCTCCTGCTGATCGCACTGAGCCTGGCTCTCGTGACCAACAGC |
| | GACATCCAGATGACACAGAGCCCCAGCACACTGTCTGCCAGCGTGGGAGACAGAGTGACC |
| | ATCACCTGTAGAGCCAGCCAGTCCATCTCCTCTTGGCTGGCCTGGTATCAGCAAAAGCCT |
| | GGCAAGGCCCCTAAGCTGCTGATCTACAAGGCCTCCACACTGGAAAGCGGGGTGCCCTCC |
| | AGATTTTCTGGCAGCGGATCTGGCACCGAGTTCACCCTGACCATCAGTAGCCTGCAGCCT |
| | GACGACTTCGCCACCTACTACTGCCAGCAGTACAATACCTACTGGACCTTCGGCCAGGGA |
| | ACAAAGGTGGAAATCAAGCGGACTGTGGCCGCTCCAAGCGTGTTCATCTTTCCACCTAGC |
| | GACGAGCAGCTGAAGTCCGGCACAGCCTCTGTTGTGTGCCTGCTGAACAACTTCTACCCC |
| | AGAGAAGCCAAGGTGCAGTGGAAAGTGGACAATGCCCTGCAGAGCGGCAACTCCCAAGAG |
| | AGCGTGACAGAGCAGGACTCCAAGGATTCCACCTACAGCCTGTCTAGCACCCTGACACTG |
| | AGCAAGGCCGACTACGAGAAGCACAAAGTGTATGCCTGCGAAGTGACCCACCAGGGCCTT |
| | AGCTCTCCAGTGACCAAGAGCTTCAACCGGGGCGAGTGTTGATAA |
| L02 | ATGTACAGAATGCAGCTGCTGCTGCTCATTGCCCTGTCTCTGGCCCTGGTCACCAATTCT |
| SEQ ID NO: 286 | GAGGTCCAGCTGCTTGAGAGTGGTGGTGGACTGGTTCAGCCTGGTGGCAGCCTGAGACTG |
| | TCTTGTGCTGCCTCTGGCTTCACCTTCAGCCACTATATCATGATGTGGGTCAGACAGGCC |
| | CCTGGCAAAGGCCTGGAATGGGTGTCAGGCATCTACAGCAGTGGTGGCATCACAGTGTAT |
| | GCTGACTCTGTGAAGGGCAGATTCACCATCAGCAGAGACAACAGCAAGAACACCCTGTAC |
| | CTGCAGATGAACTCCCTGAGAGCTGAGGACACAGCAGTGTACTACTGTGCCTATAGAAGA |
| | ATTGGGGTGCCCAGAAGGGATGAGTTTGACATCTGGGGCCAGGGCACCATGGTTACAGTG |
| | TCCTCTGCCAGCACAAAGGGCCCCTCTGTTTTTCCACTGGCTCCCAGCAGCAAGAGCACC |
| | AGTGGTGGAACAGCTGCCCTGGGCTGTCTGGTCAAGGATTACTTCCCTGAGCCTGTGACT |
| | GTGTCCTGGAACTCTGGGGCTCTGACTTCTGGGGTGCACACCTTTCCAGCTGTGCTGCAG |
| | TCCTCTGGCCTGTACTCTCTGTCCTCTGTGGTCACAGTGCCTAGCTCTAGCCTGGGCACA |
| | CAGACCTACATCTGCAATGTGAACCACAAGCCTAGCAACACCAAGGTGGACAAGAGAGTG |
| | GAACCCAAGAGCTGTGACAAGACCCACACCTGTCCTCCATGTCCTGCTCCAGAACTGCTT |
| | GGAGGCCCTTCTGTGTTCCTGTTTCCTCCAAAGCCTAAGGACACCCTGATGATCAGCAGA |
| | ACCCCTGAAGTGACCTGTGTGGTGGTTGATGTGTCCCATGAGGACCCAGAAGTGAAGTTC |
| | AATTGGTATGTGGATGGGGTTGAAGTGCACAATGCCAAGACCAAGCCTAGAGAGGAACAG |
| | TACAACAGCACCTACAGAGTGGTTTCTGTGCTGACAGTGCTGCACCAGGACTGGCTGAAT |
| | GGCAAAGAGTACAAGTGCAAGGTGTCCAACAAGGCCCTGCCTGCTCCTATTGAGAAAACC |
| | ATCTCCAAGGCCAAGGGCCAGCCAAGAGAACCCCAGGTTTACACCCTGCCACCTAGCAGA |
| | GAAGAGATGACCAAGAACCAGGTGTCCCTGACCTGCCTGGTTAAGGGCTTCTACCCCTCT |
| | GACATTGCTGTGGAATGGGAGAGCAATGGCCAGCCTGAAAACAACTACAAGACAACCCCT |
| | CCTGTGCTGGACTCTGATGGCTCATTCTTCCTGTACAGCAAGCTGACTGTGGACAAGTCC |
| | AGATGGCAGCAGGGAAATGTGTTCAGCTGCTCTGTGATGCATGAGGCCCTGCACAACCAC |
| | TACACCCAGAAAAGTCTGTCTCTGTCCCCTGGCAGAAAGAGAAGAGGCTCTGGAGAAGGC |
| | AGAGGCTCCCTGCTGACATGTGGGGATGTTGAAGAGAATCCTGGGCCTATGTATAGGATG |
| | CAACTGCTCCTCCTGATTGCTCTGAGCCTGGCTCTTGTGACCAACTCTGACATCCAGATG |
| | ACACAGAGCCCCTCCACACTGTCTGCATCTGTGGGAGACAGAGTGACCATCACCTGTAGA |
| | GCCAGCCAGTCTATCTCTAGCTGGCTGGCCTGGTATCAGCAAAAGCCTGGCAAGGCCCCT |
| | AAGCTGCTGATCTACAAGGCCAGCACACTTGAGTCAGGGGTGCCCTCCAGATTTTCTGGC |
| | TCTGGATCTGGCACAGAGTTCACCCTGACCATCAGCTCCCTGCAGCCAGATGACTTTGCC |
| | ACCTACTACTGCCAGCAGTACAATACCTACTGGACCTTTGGCCAGGGAACAAAGGTGGAA |
| | ATCAAGAGAACAGTGGCTGCCCCATCTGTGTTCATCTTCCCACCATCTGATGAACAGCTG |
| | AAGTCTGGCACTGCCTCTGTTGTGTGCCTGCTGAACAACTTTTACCCCAGAGAAGCCAAG |
| | GTGCAGTGGAAAGTGGATAATGCCCTGCAGTCTGGCAATAGCCAAGAATCTGTGACAGAG |
| | CAGGACTCCAAGGATTCCACCTACAGCCTGAGCAGCACCCTGACACTGAGCAAGGCTGAC |
| | TATGAGAAGCACAAAGTGTATGCCTGTGAAGTGACACACCAGGGACTGAGCAGCCCAGTG |
| | ACCAAGAGCTTCAACAGGGGAGAGTGCTGATAA |
| L03 | ATGTACAGAATGCAGCTGCTGCTGCTCATTGCCCTGTCTCTGGCCCTGGTCACCAATTCT |
| SEQ ID NO: 287 | GAGGTCCAGCTGCTTGAGAGTGGTGGTGGACTGGTTCAGCCTGGTGGCAGCCTGAGACTG |
| | TCTTGTGCTGCCTCTGGCTTCACCTTCAGCCACTATATCATGATGTGGGTCAGACAGGCC |
| | CCTGGCAAAGGCCTGGAATGGGTGTCAGGCATCTACAGCAGTGGTGGCATCACAGTGTAT |
| | GCTGACTCTGTGAAGGGCAGATTCACCATCAGCAGAGACAACAGCAAGAACACCCTGTAC |
| | CTGCAGATGAACTCCCTGAGAGCTGAGGACACAGCAGTGTACTACTGTGCCTATAGAAGA |
| | ATTGGGGTGCCCAGAAGGGATGAGTTTGACATCTGGGGCCAGGGCACCATGGTTACAGTG |
| | TCCTCTGCCAGCACAAAGGGCCCCTCTGTTTTTCCACTGGCTCCCAGCAGCAAGAGCACC |
| | AGTGGTGGAACAGCTGCCCTGGGCTGTCTGGTCAAGGATTACTTCCCTGAGCCTGTGACT |
| | GTGTCCTGGAACTCTGGGGCTCTGACCTCTGGGGTGCACACCTTTCCAGCTGTGCTGCAG |
| | TCCTCTGGCCTGTACTCTCTGTCCTCTGTGGTCACAGTGCCTAGCTCTAGCCTGGGCACA |
| | CAGACCTACATCTGCAATGTGAACCACAAGCCTAGCAACACCAAGGTGGACAAGAGAGTG |
| | GAACCCAAGAGCTGTGACAAGACCCACACCTGTCCTCCATGTCCTGCTCCAGAACTGCTT |
| | GGAGGCCCTTCTGTGTTCCTGTTTCCTCCAAAGCCTAAGGACACCCTGATGATCAGCAGA |
| | ACCCCTGAAGTGACCTGTGTGGTGGTTGATGTGTCCCATGAGGACCCAGAAGTGAAGTTC |
| | AATTGGTATGTGGATGGGGTTGAAGTGCACAATGCCAAGACCAAGCCTAGAGAGGAACAG |
| | TACAACAGCACCTACAGAGTGGTTTCTGTGCTGACAGTGCTGCACCAGGACTGGCTGAAT |
| | GGCAAAGAGTACAAGTGCAAGGTGTCCAACAAGGCCCTGCCTGCTCCTATTGAGAAAACC |
| | ATCTCCAAGGCCAAGGGCCAGCCAAGAGAACCCCAGGTTTACACCCTGCCACCTAGCAGA |
| | GAAGAGATGACCAAGAACCAGGTGTCCCTGACCTGCCTGGTTAAGGGCTTCTACCCCTCT |
| | GACATTGCTGTGGAATGGGAGAGCAATGGCCAGCCTGAAAACAACTACAAGACAACCCCT |
| | CCTGTGCTGGACTCTGATGGCTCATTCTTCCTGTACAGCAAGCTGACTGTGGACAAGTCC |
| | AGATGGCAGCAGGGAAATGTGTTCAGCTGCTCTGTGATGCATGAGGCCCTGCACAACCAC |
| | TACACCCAGAAAAGTCTGTCTCTGTCCCCTGGCAGAAAGAGAAGAGGATCAGGGGCCCCA |
| | GTGAAGCAGACCCTGAACTTTGATCTGCTGAAGCTGGCTGGGGATGTTGAGAGCAACCCT |
| | GGACCTATGTATAGGATGCAACTGCTCCTCCTGATTGCTCTGAGCCTGGCTCTTGTGACC |
| | AACTCTGACATCCAGATGACACAGAGCCCCTCCACACTGTCTGCATCTGTGGGAGACAGA |
| | GTGACCATCACCTGTAGAGCCAGCCAGTCTATCTCTTCCTGGCTGGCCTGGTATCAGCAA |
| | AAGCCTGGCAAGGCCCCTAAGCTGCTGATCTACAAGGCCAGCACACTTGAGTCAGGGGTG |
| | CCCTCCAGATTTTCTGGCTCTGGATCTGGCACAGAGTTCACCCTGACCATCAGCTCCCTG |
| | CAGCCAGATGACTTTGCCACCTACTACTGCCAGCAGTACAATACCTACTGGACCTTTGGC |
| | CAGGGAACAAAGGTGGAAATCAAGAGAACAGTGGCTGCCCCATCTGTGTTCATCTTCCCA |
| | CCATCTGATGAACAGCTGAAGTCTGGCACTGCCTCTGTTGTGTGCCTGCTGAACAACTTT |
| | TACCCTAGAGAAGCCAAGGTGCAGTGGAAAGTGGATAATGCCCTGCAGTCTGGCAATAGC |
| | CAAGAATCTGTGACAGAGCAGGACTCCAAGGATTCCACCTACAGCCTGAGCAGCACCCTG |
| | ACACTGAGCAAGGCTGACTATGAGAAGCACAAAGTGTATGCCTGTGAAGTGACACACCAG |
| | GGACTGAGCAGCCCAGTGACCAAGAGCTTCAACAGGGGAGAGTGCTGATAA |
| CAG.LAN.F2A | ctagtcgacattgattattgactagttattaatagtaatcaattacggggtcattagttc |
| (CAG.L01) | atagcccatatatggagttccgcgttacataacttacggtaaatggcccgcctggctgac |
| SEQ ID NO: 435 | cgcccaacgacccccgcccattgacgtcaataatgacgtatgttcccatagtaacgccaa |
| | tagggactttccattgacgtcaatgggtggagtatttacggtaaactgcccacttggcag |
| | tacatcaagtgtatcatatgccaagtacgccccctattgacgtcaatgacggtaaatggc |
| | ccgcctggcattatgcccagtacatgaccttatgggactttcctacttggcagtacatct |
| | acgtattagtcatcgctattaccatggtcgaggtgagccccacgttctgcttcactctcc |
| | ccatctcccccccctccccacccccaattttgtatttatttattttttaattattttgtg |
| | cagcgatgggggcggggggggggggggggcgcgcgccaggcggggcggggcggggcgagg |
| | ggcggggcggggcgaggcggagaggtgcggcggcagccaatcagagcggcgcgctccgaa |
| | agtttccttttatggcgaggcggcggcggcggcggccctataaaaagcgaagcgcgcggc |
| | gggcgggagtcgctgcgcgctgccttcgccccgtgccccgctccgccgccgcctcgcgcc |
| | gcccgccccggctctgactgaccgcgttactcccacaggtgagcgggcgggacggccctt |
| | ctcctccgggctgtaattagcgcttggtttaatgacggcttgtttcttttctgtggctgc |
| | gtgaaagccttgaggggctccgggagggccctttgtgcggggggagcggctcggggggtg |
| | cgtgcgtgtgtgtgtgcgtggggagcgccgcgtgcggctccgcgctgcccggcggctgtg |
| | agcgctgcgggcgcggcgcggggctttgtgcgctccgcagtgtgcgcgaggggagcgcgg |
| | ccgggggcggtgccccgcggtgcggggggggctgcgaggggaacaaaggctgcgtgcggg |
| | gtgtgtgcgtgggggggtgagcagggggtgtgggcgcgtcggtcgggctgcaaccccccc |
| | tgcacccccctccccgagttgctgagcacggcccggcttcgggtgcggggctccgtacgg |
| | ggcgtggcgcggggctcgccgtgccgggcggggggtggcggcaggtgggggtgccgggcg |
| | gggcggggccgcctcgggccggggagggctcgggggaggggcgcggcggcccccggagcg |
| | ccggcggctgtcgaggcgcggcgagccgcagccattgccttttatggtaatcgtgcgaga |
| | gggcgcagggacttcctttgtcccaaatctgtgcggagccgaaatctgggaggcgccgcc |
| | gcaccccctctagcgggcgcggggcgaagcggtgcggcgccggcaggaaggaaatgggcg |
| | gggagggccttcgtgcgtcgccgcgccgccgtccccttctccctctccagcctcggggct |
| | gtccgcggggggacggctgccttcgggggggacggggcagggcggggttcggcttctggc |
| | gtgtgaccggcggctctagagcctctgctaaccatgttcatgccttcttctttttcctac |
| | agctcctgggcaacgtgctggttattgtgctgtctcatcattttggcaaagaattcgcta |
| | gcgggcactttgcactggaacttacaacacccgagcaaggacgcgactctccaccatgta |
| | ccggatgcagctgctgctgctcattgccctgtctctggccctggtcaccaatagcgaagt |
| | ccagctgctggaatctggcggcggacttgttcaacctggcggctctctgagactgagctg |
| | tgccgcttccggcttcaccttcagccactatatcatgatgtgggtccgacaggcccctgg |
| | caaaggccttgaatgggtgtccggcatctatagcagcggcggcatcacagtgtacgccga |
| | ctctgtgaagggcagattcaccatcagccgggacaacagcaagaacaccctgtacctgca |
| | gatgaacagcctgagagccgaggacaccgccgtgtactactgcgcctatagaagaatcgg |
| | cgtgcccagacgggacgagttcgatatttggggccagggcaccatggtcaccgtgtctag |
| | cgcctctacaaagggccctagcgtgttccctctggctcctagcagcaagagcacaagcgg |
| | aggaacagccgctctgggctgtctggtcaaggactactttcccgagcctgtgaccgtgtc |
| | ctggaatagcggagcactgacaagcggcgtgcacacctttccagctgtgctgcaaagcag |
| | cggcctgtactctctgagcagcgtggtcacagtgccaagctctagcctgggcacccagac |
| | ctacatctgcaatgtgaaccacaagcctagcaacaccaaggtggacaagagagtggaacc |
| | caagagctgcgacaagacccacacctgtcctccatgtcctgctccagaactgctcggcgg |
| | accttccgtgttcctgtttcctccaaagcctaaggacaccctgatgatcagcagaacccc |
| | tgaagtgacctgcgtggtggtggatgtgtcccacgaggatcccgaagtgaagttcaattg |
| | gtacgtggacggcgtggaagtgcacaacgccaagaccaagcctagagaggaacagtacaa |
| | cagcacctacagagtggtgtccgtgctgaccgtgctgcaccaggattggctgaacggcaa |
| | agagtacaagtgcaaggtgtccaacaaggccctgcctgctcctatcgagaaaaccatcag |
| | caaggccaagggccagcctagggaaccccaggtttacacactgcctccaagccgggaaga |
| | gatgaccaagaaccaggtgtccctgacctgcctcgtgaagggcttctacccttccgatat |
| | cgccgtggaatgggagagcaatggccagccagagaacaactacaagacaacccctcctgt |
| | gctggacagcgacggctcattcttcctgtacagcaagctgacagtggacaagtccagatg |
| | gcagcagggcaacgtgttcagctgcagcgtgatgcacgaggccctgcacaaccactacac |
| | ccagaagagtctgagcctgtctccaggcctgcggaagagaagggctcctgtgaagcagac |
| | cctgaacttcgacctgctgaaactggccggcgacgtggaaagcaaccccggacctatgta |
| | tagaatgcagctcctcctgctgatcgcactgagcctggctctcgtgaccaacagcgacat |
| | ccagatgacacagagccccagcacactgtctgccagcgtgggagacagagtgaccatcac |
| | ctgtagagccagccagtccatctcctcttggctggcctggtatcagcaaaagcctggcaa |
| | ggcccctaagctgctgatctacaaggcctccacactggaaagcggggtgccctccagatt |
| | ttctggcagcggatctggcaccgagttcaccctgaccatcagtagcctgcagcctgacga |
| | cttcgccacctactactgccagcagtacaatacctactggaccttcggccagggaacaaa |
| | ggtggaaatcaagcggactgtggccgctccaagcgtgttcatctttccacctagcgacga |
| | gcagctgaagtccggcacagcctctgttgtgtgcctgctgaacaacttctaccccagaga |
| | agccaaggtgcagtggaaagtggacaatgccctgcagagcggcaactcccaagagagcgt |
| | gacagagcaggactccaaggattccacctacagcctgtctagcaccctgacactgagcaa |
| | ggccgactacgagaagcacaaagtgtatgcctgcgaagtgacccaccagggccttagctc |
| | tccagtgaccaagagcttcaaccggggcgagtgttgataaagcggccgcggtacctctag |
| | agtcgacccgggcggcctcgaggacggggtgaactacgcctgaggatccgatctttttcc |
| | ctctgccaaaaattatggggacatcatgaagccccttgagcatctgacttctggctaata |
| | aaggaaatttattttcattgcaatagtgtgttggaattttttgtgtctctcactcg |
| CAG.LANv2.F2A | gacattgattattgactagttattaatagtaatcaattacggggtcattagttcatagcc |
| (CAG.L03) | catatatggagttccgcgttacataacttacggtaaatggcccgcctggctgaccgccca |
| SEQ ID NO: 436 | acgacccccgcccattgacgtcaataatgacgtatgttcccatagtaacgccaataggga |
| | ctttccattgacgtcaatgggtggagtatttacggtaaactgcccacttggcagtacatc |
| | aagtgtatcatatgccaagtacgccccctattgacgtcaatgacggtaaatggcccgcct |
| | ggcattatgcccagtacatgaccttatgggactttcctacttggcagtacatctacgtat |
| | tagtcatcgctattaccatggtcgaggtgagccccacgttctgcttcactctccccatct |
| | cccccccctccccacccccaattttgtatttatttattttttaattattttgtgcagcga |
| | tgggggcggggggggggggggggcgcgcgccaggcggggcggggcggggcgaggggcggg |
| | gcggggcgaggcggagaggtgcggcggcagccaatcagagcggcgcgctccgaaagtttc |
| | cttttatggcgaggcggcggcggcggcggccctataaaaagcgaagcgcgcggcgggcgg |
| | gagtcgctgcgcgctgccttcgccccgtgccccgctccgccgccgcctcgcgccgcccgc |
| | cccggctctgactgaccgcgttactcccacaggtgagcgggcgggacggcccttctcctc |
| | cgggctgtaattagcgcttggtttaatgacggcttgtttcttttctgtggctgcgtgaaa |
| | gccttgaggggctccgggagggccctttgtgcggggggagcggctcggggggtgcgtgcg |
| | tgtgtgtgtgcgtggggagcgccgcgtgcggctccgcgctgcccggcggctgtgagcgct |
| | gcgggcgcggcgcggggctttgtgcgctccgcagtgtgcgcgaggggagcgcggccgggg |
| | gcggtgccccgcggtgcggggggggctgcgaggggaacaaaggctgcgtgcggggtgtgt |
| | gcgtgggggggtgagcagggggtgtgggcgcgtcggtcgggctgcaaccccccctgcacc |
| | cccctccccgagttgctgagcacggcccggcttcgggtgcggggctccgtacggggcgtg |
| | gcgcggggctcgccgtgccgggcggggggtggcggcaggtgggggtgccgggcggggcgg |
| | ggccgcctcgggccggggagggctcgggggaggggcgcggcggcccccggagcgccggcg |
| | gctgtcgaggcgcggcgagccgcagccattgccttttatggtaatcgtgcgagagggcgc |
| | agggacttcctttgtcccaaatctgtgcggagccgaaatctgggaggcgccgccgcaccc |
| | cctctagcgggcgcggggcgaagcggtgcggcgccggcaggaaggaaatgggcggggagg |
| | gccttcgtgcgtcgccgcgccgccgtccccttctccctctccagcctcggggctgtccgc |
| | ggggggacggctgccttcgggggggacggggcagggcggggttcggcttctggcgtgtga |
| | ccggcggctctagagcctctgctaaccatgttcatgccttcttctttttcctacagctcc |
| | tgggcaacgtgctggttattgtgctgtctcatcattttggcaaagaattcgccgccacca |
| | tgtacagaatgcagctgctgctgctcattgccctgtctctggccctggtcaccaattctg |
| | aggtccagctgcttgagagtggtggtggactggttcagcctggtggcagcctgagactgt |
| | cttgtgctgcctctggcttcaccttcagccactatatcatgatgtgggtcagacaggccc |
| | ctggcaaaggcctggaatgggtgtcaggcatctacagcagtggtggcatcacagtgtatg |
| | ctgactctgtgaagggcagattcaccatcagcagagacaacagcaagaacaccctgtacc |
| | tgcagatgaactccctgagagctgaggacacagcagtgtactactgtgcctatagaagaa |
| | ttggggtgcccagaagggatgagtttgacatctggggccagggcaccatggttacagtgt |
| | cctctgccagcacaaagggcccctctgtttttccactggctcccagcagcaagagcacca |
| | gtggtggaacagctgccctgggctgtctggtcaaggattacttccctgagcctgtgactg |
| | tgtcctggaactctggggctctgacctctggggtgcacacctttccagctgtgctgcagt |
| | cctctggcctgtactctctgtcctctgtggtcacagtgcctagctctagcctgggcacac |
| | agacctacatctgcaatgtgaaccacaagcctagcaacaccaaggtggacaagagagtgg |
| | aacccaagagctgtgacaagacccacacctgtcctccatgtcctgctccagaactgcttg |
| | gaggcccttctgtgttcctgtttcctccaaagcctaaggacaccctgatgatcagcagaa |
| | cccctgaagtgacctgtgtggtggttgatgtgtcccatgaggacccagaagtgaagttca |
| | attggtatgtggatggggttgaagtgcacaatgccaagaccaagcctagagaggaacagt |
| | acaacagcacctacagagtggtttctgtgctgacagtgctgcaccaggactggctgaatg |
| | gcaaagagtacaagtgcaaggtgtccaacaaggccctgcctgctcctattgagaaaacca |
| | tctccaaggccaagggccagccaagagaaccccaggtttacaccctgccacctagcagag |
| | aagagatgaccaagaaccaggtgtccctgacctgcctggttaagggcttctacccctctg |
| | acattgctgtggaatgggagagcaatggccagcctgaaaacaactacaagacaacccctc |
| | ctgtgctggactctgatggctcattcttcctgtacagcaagctgactgtggacaagtcca |
| | gatggcagcagggaaatgtgttcagctgctctgtgatgcatgaggccctgcacaaccact |
| | acacccagaaaagtctgtctctgtcccctggcagaaagagaagaggatcaggggccccag |
| | tgaagcagaccctgaactttgatctgctgaagctggctggggatgttgagagcaaccctg |
| | gacctatgtataggatgcaactgctcctcctgattgctctgagcctggctcttgtgacca |
| | actctgacatccagatgacacagagcccctccacactgtctgcatctgtgggagacagag |
| | tgaccatcacctgtagagccagccagtctatctcttcctggctggcctggtatcagcaaa |
| | agcctggcaaggcccctaagctgctgatctacaaggccagcacacttgagtcaggggtgc |
| | cctccagattttctggctctggatctggcacagagttcaccctgaccatcagctccctgc |
| | agccagatgactttgccacctactactgccagcagtacaatacctactggacctttggcc |
| | agggaacaaaggtggaaatcaagagaacagtggctgccccatctgtgttcatcttcccac |
| | catctgatgaacagctgaagtctggcactgcctctgttgtgtgcctgctgaacaactttt |
| | accctagagaagccaaggtgcagtggaaagtggataatgccctgcagtctggcaatagcc |
| | aagaatctgtgacagagcaggactccaaggattccacctacagcctgagcagcaccctga |
| | cactgagcaaggctgactatgagaagcacaaagtgtatgcctgtgaagtgacacaccagg |
| | gactgagcagcccagtgaccaagagcttcaacaggggagagtgctgataactcgaggacg |
| | gggtgaactacgcctgaggatccgatctttttccctctgccaaaaattatggggacatca |
| | tgaagccccttgagcatctgacttctggctaataaaggaaatttattttcattgcaatag |
| | tgtgttggaattttttgtgtctctca |
| CAG.LANv2.T2A | gacattgattattgactagttattaatagtaatcaattacggggtcattagttcatagcc |
| (CAG.L02) | catatatggagttccgcgttacataacttacggtaaatggcccgcctggctgaccgccca |
| SEQ ID NO: 437 | acgacccccgcccattgacgtcaataatgacgtatgttcccatagtaacgccaataggga |
| | ctttccattgacgtcaatgggtggagtatttacggtaaactgcccacttggcagtacatc |
| | aagtgtatcatatgccaagtacgccccctattgacgtcaatgacggtaaatggcccgcct |
| | ggcattatgcccagtacatgaccttatgggactttcctacttggcagtacatctacgtat |
| | tagtcatcgctattaccatggtcgaggtgagccccacgttctgcttcactctccccatct |
| | cccccccctccccacccccaattttgtatttatttattttttaattattttgtgcagcga |
| | tgggggcggggggggggggggggcgcgcgccaggcggggcggggcggggcgaggggcggg |
| | gcggggcgaggcggagaggtgcggcggcagccaatcagagcggcgcgctccgaaagtttc |
| | cttttatggcgaggcggcggcggcggcggccctataaaaagcgaagcgcgcggcgggcgg |
| | gagtcgctgcgcgctgccttcgccccgtgccccgctccgccgccgcctcgcgccgcccgc |
| | cccggctctgactgaccgcgttactcccacaggtgagcgggcgggacggcccttctcctc |
| | cgggctgtaattagcgcttggtttaatgacggcttgtttcttttctgtggctgcgtgaaa |
| | gccttgaggggctccgggagggccctttgtgcggggggagcggctcggggggtgcgtgcg |
| | tgtgtgtgtgcgtggggagcgccgcgtgcggctccgcgctgcccggcggctgtgagcgct |
| | gcgggcgcggcgcggggctttgtgcgctccgcagtgtgcgcgaggggagcgcggccgggg |
| | gcggtgccccgcggtgcggggggggctgcgaggggaacaaaggctgcgtgcggggtgtgt |
| | gcgtgggggggtgagcagggggtgtgggcgcgtcggtcgggctgcaaccccccctgcacc |
| | cccctccccgagttgctgagcacggcccggcttcgggtgcggggctccgtacggggcgtg |
| | gcgcggggctcgccgtgccgggcggggggtggcggcaggtgggggtgccgggcggggcgg |
| | ggccgcctcgggccggggagggctcgggggaggggcgcggcggcccccggagcgccggcg |
| | gctgtcgaggcgcggcgagccgcagccattgccttttatggtaatcgtgcgagagggcgc |
| | agggacttcctttgtcccaaatctgtgcggagccgaaatctgggaggcgccgccgcaccc |
| | cctctagcgggcgcggggcgaagcggtgcggcgccggcaggaaggaaatgggcggggagg |
| | gccttcgtgcgtcgccgcgccgccgtccccttctccctctccagcctcggggctgtccgc |
| | ggggggacggctgccttcgggggggacggggcagggcggggttcggcttctggcgtgtga |
| | ccggcggctctagagcctctgctaaccatgttcatgccttcttctttttcctacagctcc |
| | tgggcaacgtgctggttattgtgctgtctcatcattttggcaaagaattcgccgccacca |
| | tgtacagaatgcagctgctgctgctcattgccctgtctctggccctggtcaccaattctg |
| | aggtccagctgcttgagagtggtggtggactggttcagcctggtggcagcctgagactgt |
| | cttgtgctgcctctggcttcaccttcagccactatatcatgatgtgggtcagacaggccc |
| | ctggcaaaggcctggaatgggtgtcaggcatctacagcagtggtggcatcacagtgtatg |
| | ctgactctgtgaagggcagattcaccatcagcagagacaacagcaagaacaccctgtacc |
| | tgcagatgaactccctgagagctgaggacacagcagtgtactactgtgcctatagaagaa |
| | ttggggtgcccagaagggatgagtttgacatctggggccagggcaccatggttacagtgt |
| | cctctgccagcacaaagggcccctctgtttttccactggctcccagcagcaagagcacca |
| | gtggtggaacagctgccctgggctgtctggtcaaggattacttccctgagcctgtgactg |
| | tgtcctggaactctggggctctgacttctggggtgcacacctttccagctgtgctgcagt |
| | cctctggcctgtactctctgtcctctgtggtcacagtgcctagctctagcctgggcacac |
| | agacctacatctgcaatgtgaaccacaagcctagcaacaccaaggtggacaagagagtgg |
| | aacccaagagctgtgacaagacccacacctgtcctccatgtcctgctccagaactgcttg |
| | gaggcccttctgtgttcctgtttcctccaaagcctaaggacaccctgatgatcagcagaa |
| | cccctgaagtgacctgtgtggtggttgatgtgtcccatgaggacccagaagtgaagttca |
| | attggtatgtggatggggttgaagtgcacaatgccaagaccaagcctagagaggaacagt |
| | acaacagcacctacagagtggtttctgtgctgacagtgctgcaccaggactggctgaatg |
| | gcaaagagtacaagtgcaaggtgtccaacaaggccctgcctgctcctattgagaaaacca |
| | tctccaaggccaagggccagccaagagaaccccaggtttacaccctgccacctagcagag |
| | aagagatgaccaagaaccaggtgtccctgacctgcctggttaagggcttctacccctctg |
| | acattgctgtggaatgggagagcaatggccagcctgaaaacaactacaagacaacccctc |
| | ctgtgctggactctgatggctcattcttcctgtacagcaagctgactgtggacaagtcca |
| | gatggcagcagggaaatgtgttcagctgctctgtgatgcatgaggccctgcacaaccact |
| | acacccagaaaagtctgtctctgtcccctggcagaaagagaagaggctctggagaaggca |
| | gaggctccctgctgacatgtggggatgttgaagagaatcctgggcctatgtataggatgc |
| | aactgctcctcctgattgctctgagcctggctcttgtgaccaactctgacatccagatga |
| | cacagagcccctccacactgtctgcatctgtgggagacagagtgaccatcacctgtagag |
| | ccagccagtctatctctagctggctggcctggtatcagcaaaagcctggcaaggccccta |
| | agctgctgatctacaaggccagcacacttgagtcaggggtgccctccagattttctggct |
| | ctggatctggcacagagttcaccctgaccatcagctccctgcagccagatgactttgcca |
| | cctactactgccagcagtacaatacctactggacctttggccagggaacaaaggtggaaa |
| | tcaagagaacagtggctgccccatctgtgttcatcttcccaccatctgatgaacagctga |
| | agtctggcactgcctctgttgtgtgcctgctgaacaacttttaccccagagaagccaagg |
| | tgcagtggaaagtggataatgccctgcagtctggcaatagccaagaatctgtgacagagc |
| | aggactccaaggattccacctacagcctgagcagcaccctgacactgagcaaggctgact |
| | atgagaagcacaaagtgtatgcctgtgaagtgacacaccagggactgagcagcccagtga |
| | ccaagagcttcaacaggggagagtgctgataactcgaggacggggtgaactacgcctgag |
| | gatccgatctttttccctctgccaaaaattatggggacatcatgaagccccttgagcatc |
| | tgacttctggctaataaaggaaatttattttcattgcaatagtgtgttggaattttttgt |
| | gtctctcactcg |
| TBG.LANv2.T2A | aggttaatttttaaaaagcagtcaaaagtccaagtggcccttggcagcatttactctctc |
| (TBG.L02) | tgtttgctctggttaataatctcaggagcacaaacattccagatccaggttaatttttaa |
| SEQ ID NO: 438 | aaagcagtcaaaagtccaagtggcccttggcagcatttactctctctgtttgctctggtt |
| | aataatctcaggagcacaaacattccagatccggcgcgccagggctggaagctacctttg |
| | acatcatttcctctgcgaatgcatgtataatttctacagaacctattagaaaggatcacc |
| | cagcctctgcttttgtacaactttcccttaaaaaactgccaattccactgctgtttggcc |
| | caatagtgagaactttttcctgctgcctcttggtgcttttgcctatggcccctattctgc |
| | ctgctgaagacactcttgccagcatggacttaaacccctccagctctgacaatcctcttt |
| | ctcttttgttttacatgaagggtctggcagccaaagcaatcactcaaagttcaaacctta |
| | tcattttttgctttgttcctcttggccttggttttgtacatcagctttgaaaataccatc |
| | ccagggttaatgctggggttaatttataactaagagtgctctagttttgcaatacaggac |
| | atgctataaaaatggaaagatgttgctttctgagagacagctttattgcggtagtttatc |
| | acagttaaattgctaacgcagtcagtgcttctgacacaacagtctcgaacttaagctgca |
| | gaagttggtcgtgaggcactgggcaggtaagtatcaaggttacaagacaggtttaaggag |
| | accaatagaaactgggcttgtcgagacagagaagactcttgcgtttctgataggcaccta |
| | ttggtcttactgacatccactttgcctttctctccacaggtgtccactcccagttcaatt |
| | acagctcttaaggctagagtacttaatacgactcactataggctagcctcgagaattcac |
| | gcgtgccgccaccatgtacagaatgcagctgctgctgctcattgccctgtctctggccct |
| | ggtcaccaattctgaggtccagctgcttgagagtggtggtggactggttcagcctggtgg |
| | cagcctgagactgtcttgtgctgcctctggcttcaccttcagccactatatcatgatgtg |
| | ggtcagacaggcccctggcaaaggcctggaatgggtgtcaggcatctacagcagtggtgg |
| | catcacagtgtatgctgactctgtgaagggcagattcaccatcagcagagacaacagcaa |
| | gaacaccctgtacctgcagatgaactccctgagagctgaggacacagcagtgtactactg |
| | tgcctatagaagaattggggtgcccagaagggatgagtttgacatctggggccagggcac |
| | catggttacagtgtcctctgccagcacaaagggcccctctgtttttccactggctcccag |
| | cagcaagagcaccagtggtggaacagctgccctgggctgtctggtcaaggattacttccc |
| | tgagcctgtgactgtgtcctggaactctggggctctgacttctggggtgcacacctttcc |
| | agctgtgctgcagtcctctggcctgtactctctgtcctctgtggtcacagtgcctagctc |
| | tagcctgggcacacagacctacatctgcaatgtgaaccacaagcctagcaacaccaaggt |
| | ggacaagagagtggaacccaagagctgtgacaagacccacacctgtcctccatgtcctgc |
| | tccagaactgcttggaggcccttctgtgttcctgtttcctccaaagcctaaggacaccct |
| | gatgatcagcagaacccctgaagtgacctgtgtggtggttgatgtgtcccatgaggaccc |
| | agaagtgaagttcaattggtatgtggatggggttgaagtgcacaatgccaagaccaagcc |
| | tagagaggaacagtacaacagcacctacagagtggtttctgtgctgacagtgctgcacca |
| | ggactggctgaatggcaaagagtacaagtgcaaggtgtccaacaaggccctgcctgctcc |
| | tattgagaaaaccatctccaaggccaagggccagccaagagaaccccaggtttacaccct |
| | gccacctagcagagaagagatgaccaagaaccaggtgtccctgacctgcctggttaaggg |
| | cttctacccctctgacattgctgtggaatgggagagcaatggccagcctgaaaacaacta |
| | caagacaacccctcctgtgctggactctgatggctcattcttcctgtacagcaagctgac |
| | tgtggacaagtccagatggcagcagggaaatgtgttcagctgctctgtgatgcatgaggc |
| | cctgcacaaccactacacccagaaaagtctgtctctgtcccctggcagaaagagaagagg |
| | ctctggagaaggcagaggctccctgctgacatgtggggatgttgaagagaatcctgggcc |
| | tatgtataggatgcaactgctcctcctgattgctctgagcctggctcttgtgaccaactc |
| | tgacatccagatgacacagagcccctccacactgtctgcatctgtgggagacagagtgac |
| | catcacctgtagagccagccagtctatctctagctggctggcctggtatcagcaaaagcc |
| | tggcaaggcccctaagctgctgatctacaaggccagcacacttgagtcaggggtgccctc |
| | cagattttctggctctggatctggcacagagttcaccctgaccatcagctccctgcagcc |
| | agatgactttgccacctactactgccagcagtacaatacctactggacctttggccaggg |
| | aacaaaggtggaaatcaagagaacagtggctgccccatctgtgttcatcttcccaccatc |
| | tgatgaacagctgaagtctggcactgcctctgttgtgtgcctgctgaacaacttttaccc |
| | cagagaagccaaggtgcagtggaaagtggataatgccctgcagtctggcaatagccaaga |
| | atctgtgacagagcaggactccaaggattccacctacagcctgagcagcaccctgacact |
| | gagcaaggctgactatgagaagcacaaagtgtatgcctgtgaagtgacacaccagggact |
| | gagcagcccagtgaccaagagcttcaacaggggagagtgctgataagtcgacccgggcgg |
| | cctcgaggacggggtgaactacgcctgaggatccgatctttttccctctgccaaaaatta |
| | tggggacatcatgaagccccttgagcatctgacttctggctaataaaggaaatttatttt |
| | cattgcaatagtgtgttggaattttttgtgtctctca |
| ApoE.hAAT.LANv2.T2A | ggctcagaggcacacaggagtttctgggctcaccctgcccccttccaacccctcagttcc |
| (ApoE.hAAT.L02) | catcctccagcagctgtttgtgtgctgcctctgaagtccacactgaacaaacttcagcct |
| SEQ ID NO: 439 | actcatgtccctaaaatgggcaaacattgcaagcagcaaacagcaaacacacagccctcc |
| | ctgcctgctgaccttggagctggggcagaggtcagagacctctctgggcccatgccacct |
| | ccaacatccactcgaccccttggaatttcggtggagaggagcagaggttgtcctggcgtg |
| | gtttaggtagtgtgagaggggtacccggggatcttgctaccagtggaacagccactaagg |
| | attctgcagtgagagcagagggccagctaagtggtactctcccagagactgtctgactca |
| | cgccaccccctccaccttggacacaggacgctgtggtttctgagccaggtacaatgactc |
| | ctttcggtaagtgcagtggaagctgtacactgcccaggcaaagcgtccgggcagcgtagg |
| | cgggcgactcagatcccagccagtggacttagcccctgtttgctcctccgataactgggg |
| | tgaccttggttaatattcaccagcagcctcccccgttgcccctctggatccactgcttaa |
| | atacggacgaggacagggccctgtctcctcagcttcaggcaccaccactgacctgggaca |
| | gtcaggtaagtatcaaggttacaagacaggtttaaggagaccaatagaaactgggcttgt |
| | cgagacagagaagactcttgcgtttctgataggcacctattggtcttactgacatccact |
| | ttgcctttctctccacaggttcgaattcgccgccaccatgtacagaatgcagctgctgct |
| | gctcattgccctgtctctggccctggtcaccaattctgaggtccagctgcttgagagtgg |
| | tggtggactggttcagcctggtggcagcctgagactgtcttgtgctgcctctggcttcac |
| | cttcagccactatatcatgatgtgggtcagacaggcccctggcaaaggcctggaatgggt |
| | gtcaggcatctacagcagtggtggcatcacagtgtatgctgactctgtgaagggcagatt |
| | caccatcagcagagacaacagcaagaacaccctgtacctgcagatgaactccctgagagc |
| | tgaggacacagcagtgtactactgtgcctatagaagaattggggtgcccagaagggatga |
| | gtttgacatctggggccagggcaccatggttacagtgtcctctgccagcacaaagggccc |
| | ctctgtttttccactggctcccagcagcaagagcaccagtggtggaacagctgccctggg |
| | ctgtctggtcaaggattacttccctgagcctgtgactgtgtcctggaactctggggctct |
| | gacttctggggtgcacacctttccagctgtgctgcagtcctctggcctgtactctctgtc |
| | ctctgtggtcacagtgcctagctctagcctgggcacacagacctacatctgcaatgtgaa |
| | ccacaagcctagcaacaccaaggtggacaagagagtggaacccaagagctgtgacaagac |
| | ccacacctgtcctccatgtcctgctccagaactgcttggaggcccttctgtgttcctgtt |
| | tcctccaaagcctaaggacaccctgatgatcagcagaacccctgaagtgacctgtgtggt |
| | ggttgatgtgtcccatgaggacccagaagtgaagttcaattggtatgtggatggggttga |
| | agtgcacaatgccaagaccaagcctagagaggaacagtacaacagcacctacagagtggt |
| | ttctgtgctgacagtgctgcaccaggactggctgaatggcaaagagtacaagtgcaaggt |
| | gtccaacaaggccctgcctgctcctattgagaaaaccatctccaaggccaagggccagcc |
| | aagagaaccccaggtttacaccctgccacctagcagagaagagatgaccaagaaccaggt |
| | gtccctgacctgcctggttaagggcttctacccctctgacattgctgtggaatgggagag |
| | caatggccagcctgaaaacaactacaagacaacccctcctgtgctggactctgatggctc |
| | attcttcctgtacagcaagctgactgtggacaagtccagatggcagcagggaaatgtgtt |
| | cagctgctctgtgatgcatgaggccctgcacaaccactacacccagaaaagtctgtctct |
| | gtcccctggcagaaagagaagaggctctggagaaggcagaggctccctgctgacatgtgg |
| | ggatgttgaagagaatcctgggcctatgtataggatgcaactgctcctcctgattgctct |
| | gagcctggctcttgtgaccaactctgacatccagatgacacagagcccctccacactgtc |
| | tgcatctgtgggagacagagtgaccatcacctgtagagccagccagtctatctctagctg |
| | gctggcctggtatcagcaaaagcctggcaaggcccctaagctgctgatctacaaggccag |
| | cacacttgagtcaggggtgccctccagattttctggctctggatctggcacagagttcac |
| | cctgaccatcagctccctgcagccagatgactttgccacctactactgccagcagtacaa |
| | tacctactggacctttggccagggaacaaaggtggaaatcaagagaacagtggctgcccc |
| | atctgtgttcatcttcccaccatctgatgaacagctgaagtctggcactgcctctgttgt |
| | gtgcctgctgaacaacttttaccccagagaagccaaggtgcagtggaaagtggataatgc |
| | cctgcagtctggcaatagccaagaatctgtgacagagcaggactccaaggattccaccta |
| | cagcctgagcagcaccctgacactgagcaaggctgactatgagaagcacaaagtgtatgc |
| | ctgtgaagtgacacaccagggactgagcagcccagtgaccaagagcttcaacaggggaga |
| | gtgctgataactcgaggacggggtgaactacgcctgaggatccgatctttttccctctgc |
| | caaaaattatggggacatcatgaagccccttgagcatctgacttctggctaataaaggaa |
| | atttattttcattgcaatagtgtgttggaattttttgtgtctctca |
| LSPX1.LANv2.T2A | aggttaatttttaaaaagcagtcaaaagtccaagtggcccttggcagcatttactctctc |
| (LSPX.L02) | tgtttgctctggttaataatctcaggagcacaaacattccagatccaggttaatttttaa |
| SEQ ID NO: 440 | aaagcagtcaaaagtccaagtggcccttggcagcatttactctctctgtttgctctggtt |
| | aataatctcaggagcacaaacattccagatccggcgcgccagggctggaagctacctttg |
| | tctagaaggctcagaggcacacaggagtttctgggctcaccctgcccccttccaacccct |
| | cagttcccatcctccagcagctgtttgtgtgctgcctctgaagtccacactgaacaaact |
| | tcagcctactcatgtccctaaaatgggcaaacattgcaagcagcaaacagcaaacacaca |
| | gccctccctgcctgctgaccttggagctggggcagaggtcagagacctctctgggcccat |
| | gccacctccaacatccactcgaccccttggaatttcggtggagaggagcagaggttgtcc |
| | tggcgtggtttaggtagtgtgagaggggtacccggggatcttgctaccagtggaacagcc |
| | actaaggattctgcagtgagagcagagggccagctaagtggtactctcccagagactgtc |
| | tgactcacgccaccccctccaccttggacacaggacgctgtggtttctgagccaggtaca |
| | atgactcctttcggtaagtgcagtggaagctgtacactgcccaggcaaagcgtccgggca |
| | gcgtaggcgggcgactcagatcccagccagtggacttagcccctgtttgctcctccgata |
| | actggggtgaccttggttaatattcaccagcagcctcccccgttgcccctctggatccac |
| | tgcttaaatacggacgaggacagggccctgtctcctcagcttcaggcaccaccactgacc |
| | tgggacagtcaggtaagtatcaaggttacaagacaggtttaaggagaccaatagaaactg |
| | ggcttgtcgagacagagaagactcttgcgtttctgataggcacctattggtcttactgac |
| | atccactttgcctttctctccacaggttgaattcgccgccaccatgtacagaatgcagct |
| | gctgctgctcattgccctgtctctggccctggtcaccaattctgaggtccagctgcttga |
| | gagtggtggtggactggttcagcctggtggcagcctgagactgtcttgtgctgcctctgg |
| | cttcaccttcagccactatatcatgatgtgggtcagacaggcccctggcaaaggcctgga |
| | atgggtgtcaggcatctacagcagtggtggcatcacagtgtatgctgactctgtgaaggg |
| | cagattcaccatcagcagagacaacagcaagaacaccctgtacctgcagatgaactccct |
| | gagagctgaggacacagcagtgtactactgtgcctatagaagaattggggtgcccagaag |
| | ggatgagtttgacatctggggccagggcaccatggttacagtgtcctctgccagcacaaa |
| | gggcccctctgtttttccactggctcccagcagcaagagcaccagtggtggaacagctgc |
| | cctgggctgtctggtcaaggattacttccctgagcctgtgactgtgtcctggaactctgg |
| | ggctctgacttctggggtgcacacctttccagctgtgctgcagtcctctggcctgtactc |
| | tctgtcctctgtggtcacagtgcctagctctagcctgggcacacagacctacatctgcaa |
| | tgtgaaccacaagcctagcaacaccaaggtggacaagagagtggaacccaagagctgtga |
| | caagacccacacctgtcctccatgtcctgctccagaactgcttggaggcccttctgtgtt |
| | cctgtttcctccaaagcctaaggacaccctgatgatcagcagaacccctgaagtgacctg |
| | tgtggtggttgatgtgtcccatgaggacccagaagtgaagttcaattggtatgtggatgg |
| | ggttgaagtgcacaatgccaagaccaagcctagagaggaacagtacaacagcacctacag |
| | agtggtttctgtgctgacagtgctgcaccaggactggctgaatggcaaagagtacaagtg |
| | caaggtgtccaacaaggccctgcctgctcctattgagaaaaccatctccaaggccaaggg |
| | ccagccaagagaaccccaggtttacaccctgccacctagcagagaagagatgaccaagaa |
| | ccaggtgtccctgacctgcctggttaagggcttctacccctctgacattgctgtggaatg |
| | ggagagcaatggccagcctgaaaacaactacaagacaacccctcctgtgctggactctga |
| | tggctcattcttcctgtacagcaagctgactgtggacaagtccagatggcagcagggaaa |
| | tgtgttcagctgctctgtgatgcatgaggccctgcacaaccactacacccagaaaagtct |
| | gtctctgtcccctggcagaaagagaagaggctctggagaaggcagaggctccctgctgac |
| | atgtggggatgttgaagagaatcctgggcctatgtataggatgcaactgctcctcctgat |
| | tgctctgagcctggctcttgtgaccaactctgacatccagatgacacagagcccctccac |
| | actgtctgcatctgtgggagacagagtgaccatcacctgtagagccagccagtctatctc |
| | tagctggctggcctggtatcagcaaaagcctggcaaggcccctaagctgctgatctacaa |
| | ggccagcacacttgagtcaggggtgccctccagattttctggctctggatctggcacaga |
| | gttcaccctgaccatcagctccctgcagccagatgactttgccacctactactgccagca |
| | gtacaatacctactggacctttggccagggaacaaaggtggaaatcaagagaacagtggc |
| | tgccccatctgtgttcatcttcccaccatctgatgaacagctgaagtctggcactgcctc |
| | tgttgtgtgcctgctgaacaacttttaccccagagaagccaaggtgcagtggaaagtgga |
| | taatgccctgcagtctggcaatagccaagaatctgtgacagagcaggactccaaggattc |
| | cacctacagcctgagcagcaccctgacactgagcaaggctgactatgagaagcacaaagt |
| | gtatgcctgtgaagtgacacaccagggactgagcagcccagtgaccaagagcttcaacag |
| | gggagagtgctgataactcgaggacggggtgaactacgcctgaggatccgatctttttcc |
| | ctctgccaaaaattatggggacatcatgaagccccttgagcatctgacttctggctaata |
| | aaggaaatttattttcattgcaatagtgtgttggaattttttgtgtctctca |
| LSPX2.LANv2.T2A | aggctcagaggcacacaggagtttctgggctcaccctgcccccttccaacccctcagttc |
| (LSPX2.L02) | ccatcctccagcagctgtttgtgtgctgcctctgaagtccacactgaacaaacttcagcc |
| SEQ ID NO: 441 | tactcatgtccctaaaatgggcaaacattgcaagcagcaaacagcaaacacacagccctc |
| SEQ ID NO: | cctgcctgctgaccttggagctggggcagaggtcagagacctctctgggcccatgccacc |
| | tccaacatccactcgaccccttggaatttcggtggagaggagcagaggttgtcctggcgt |
| | ggtttaggtagtgtgagagggtctagaaggctcagaggcacacaggagtttctgggctca |
| | ccctgcccccttccaacccctcagttcccatcctccagcagctgtttgtgtgctgcctct |
| | gaagtccacactgaacaaacttcagcctactcatgtccctaaaatgggcaaacattgcaa |
| | gcagcaaacagcaaacacacagccctccctgcctgctgaccttggagctggggcagaggt |
| | cagagacctctctgggcccatgccacctccaacatccactcgaccccttggaatttcggt |
| | ggagaggagcagaggttgtcctggcgtggtttaggtagtgtgagaggggtacccggggat |
| | cttgctaccagtggaacagccactaaggattctgcagtgagagcagagggccagctaagt |
| | ggtactctcccagagactgtctgactcacgccaccccctccaccttggacacaggacgct |
| | gtggtttctgagccaggtacaatgactcctttcggtaagtgcagtggaagctgtacactg |
| | cccaggcaaagcgtccgggcagcgtaggcgggcgactcagatcccagccagtggacttag |
| | cccctgtttgctcctccgataactggggtgaccttggttaatattcaccagcagcctccc |
| | ccgttgcccctctggatccactgcttaaatacggacgaggacagggccctgtctcctcag |
| | cttcaggcaccaccactgacctgggacagtcaggtaagtatcaaggttacaagacaggtt |
| | taaggagaccaatagaaactgggcttgtcgagacagagaagactcttgcgtttctgatag |
| | gcacctattggtcttactgacatccactttgcctttctctccacaggttgaattcgccgc |
| | caccatgtacagaatgcagctgctgctgctcattgccctgtctctggccctggtcaccaa |
| | ttctgaggtccagctgcttgagagtggtggtggactggttcagcctggtggcagcctgag |
| | actgtcttgtgctgcctctggcttcaccttcagccactatatcatgatgtgggtcagaca |
| | ggcccctggcaaaggcctggaatgggtgtcaggcatctacagcagtggtggcatcacagt |
| | gtatgctgactctgtgaagggcagattcaccatcagcagagacaacagcaagaacaccct |
| | gtacctgcagatgaactccctgagagctgaggacacagcagtgtactactgtgcctatag |
| | aagaattggggtgcccagaagggatgagtttgacatctggggccagggcaccatggttac |
| | agtgtcctctgccagcacaaagggcccctctgtttttccactggctcccagcagcaagag |
| | caccagtggtggaacagctgccctgggctgtctggtcaaggattacttccctgagcctgt |
| | gactgtgtcctggaactctggggctctgacttctggggtgcacacctttccagctgtgct |
| | gcagtcctctggcctgtactctctgtcctctgtggtcacagtgcctagctctagcctggg |
| | cacacagacctacatctgcaatgtgaaccacaagcctagcaacaccaaggtggacaagag |
| | agtggaacccaagagctgtgacaagacccacacctgtcctccatgtcctgctccagaact |
| | gcttggaggcccttctgtgttcctgtttcctccaaagcctaaggacaccctgatgatcag |
| | cagaacccctgaagtgacctgtgtggtggttgatgtgtcccatgaggacccagaagtgaa |
| | gttcaattggtatgtggatggggttgaagtgcacaatgccaagaccaagcctagagagga |
| | acagtacaacagcacctacagagtggtttctgtgctgacagtgctgcaccaggactggct |
| | gaatggcaaagagtacaagtgcaaggtgtccaacaaggccctgcctgctcctattgagaa |
| | aaccatctccaaggccaagggccagccaagagaaccccaggtttacaccctgccacctag |
| | cagagaagagatgaccaagaaccaggtgtccctgacctgcctggttaagggcttctaccc |
| | ctctgacattgctgtggaatgggagagcaatggccagcctgaaaacaactacaagacaac |
| | ccctcctgtgctggactctgatggctcattcttcctgtacagcaagctgactgtggacaa |
| | gtccagatggcagcagggaaatgtgttcagctgctctgtgatgcatgaggccctgcacaa |
| | ccactacacccagaaaagtctgtctctgtcccctggcagaaagagaagaggctctggaga |
| | aggcagaggctccctgctgacatgtggggatgttgaagagaatcctgggcctatgtatag |
| | gatgcaactgctcctcctgattgctctgagcctggctcttgtgaccaactctgacatcca |
| | gatgacacagagcccctccacactgtctgcatctgtgggagacagagtgaccatcacctg |
| | tagagccagccagtctatctctagctggctggcctggtatcagcaaaagcctggcaaggc |
| | ccctaagctgctgatctacaaggccagcacacttgagtcaggggtgccctccagattttc |
| | tggctctggatctggcacagagttcaccctgaccatcagctccctgcagccagatgactt |
| | tgccacctactactgccagcagtacaatacctactggacctttggccagggaacaaaggt |
| | ggaaatcaagagaacagtggctgccccatctgtgttcatcttcccaccatctgatgaaca |
| | gctgaagtctggcactgcctctgttgtgtgcctgctgaacaacttttaccccagagaagc |
| | caaggtgcagtggaaagtggataatgccctgcagtctggcaatagccaagaatctgtgac |
| | agagcaggactccaaggattccacctacagcctgagcagcaccctgacactgagcaaggc |
| | tgactatgagaagcacaaagtgtatgcctgtgaagtgacacaccagggactgagcagccc |
| | agtgaccaagagcttcaacaggggagagtgctgataactcgaggacggggtgaactacgc |
| | ctgaggatccgatctttttccctctgccaaaaattatggggacatcatgaagccccttga |
| | gcatctgacttctggctaataaaggaaatttattttcattgcaatagtgtgttggaattt |
| | tttgtgtctctca |
| LTP1.LANv2.T2A | aggttaatttttaaaaagcagtcaaaagtccaagtggcccttggcagcatttactctctc |
| (LTP1.L02) | tgtttgctctggttaataatctcaggagcacaaacattccagatccaggttaatttttaa |
| SEQ ID NO: 442 | aaagcagtcaaaagtccaagtggcccttggcagcatttactctctctgtttgctctggtt |
| | aataatctcaggagcacaaacattccagatccggcgcgccagggctggaagctacctttg |
| | acatcatttcctctgcgaatgcatgtataatttctacagaacctattagaaaggatcacc |
| | cagcctctgcttttgtacaactttcccttaaaaaactgccaattccactgctgtttggcc |
| | caatagtgagaactttttcctgctgcctcttggtgcttttgcctatggcccctattctgc |
| | ctgctgaagacactcttgccagcatggacttaaacccctccagctctgacaatcctcttt |
| | ctcttttgttttacatgaagggtctggcagccaaagcaatcactcaaagttcaaacctta |
| | tcattttttgctttgttcctcttggccttggttttgtacatcagctttgaaaataccatc |
| | ccagggttaatgctggggttaatttataactaagagtgctctagttttgcaatacaggac |
| | atgctataaaaatggaaagatgttgctttctgagaggatcttgctaccagtggaacagcc |
| | actaaggattctgcagtgagagcagagggccagctaagtggtactctcccagagactgtc |
| | tgactcacgccaccccctccaccttggacacaggacgctgtggtttctgagccaggtaca |
| | gtgactcctttcggtaagtgcagtggaagctgtacactgcccaggcaaagcgtccgggca |
| | gcgtaggcgggcgactcagatcccagccagtggacttagcccctgtttgctcctccgata |
| | actggggtgaccttggttaatattcaccagcagcctcccccgttgcccctctggatccac |
| | tgcttaaatacggacgaggacagggccctgtctcctcagcttcaggcaccaccactgacc |
| | tgggacagtcaggtaagtatcaaggttacaagacaggtttaaggagaccaatagaaactg |
| | ggcttgtcgagacagagaagactcttgcgtttctgataggcacctattggtcttactgac |
| | atccactttgcctttctctccacaggttgaattcgccgccaccatgtacagaatgcagct |
| | gctgctgctcattgccctgtctctggccctggtcaccaattctgaggtccagctgcttga |
| | gagtggtggtggactggttcagcctggtggcagcctgagactgtcttgtgctgcctctgg |
| | cttcaccttcagccactatatcatgatgtgggtcagacaggcccctggcaaaggcctgga |
| | atgggtgtcaggcatctacagcagtggtggcatcacagtgtatgctgactctgtgaaggg |
| | cagattcaccatcagcagagacaacagcaagaacaccctgtacctgcagatgaactccct |
| | gagagctgaggacacagcagtgtactactgtgcctatagaagaattggggtgcccagaag |
| | ggatgagtttgacatctggggccagggcaccatggttacagtgtcctctgccagcacaaa |
| | gggcccctctgtttttccactggctcccagcagcaagagcaccagtggtggaacagctgc |
| | cctgggctgtctggtcaaggattacttccctgagcctgtgactgtgtcctggaactctgg |
| | ggctctgacttctggggtgcacacctttccagctgtgctgcagtcctctggcctgtactc |
| | tctgtcctctgtggtcacagtgcctagctctagcctgggcacacagacctacatctgcaa |
| | tgtgaaccacaagcctagcaacaccaaggtggacaagagagtggaacccaagagctgtga |
| | caagacccacacctgtcctccatgtcctgctccagaactgcttggaggcccttctgtgtt |
| | cctgtttcctccaaagcctaaggacaccctgatgatcagcagaacccctgaagtgacctg |
| | tgtggtggttgatgtgtcccatgaggacccagaagtgaagttcaattggtatgtggatgg |
| | ggttgaagtgcacaatgccaagaccaagcctagagaggaacagtacaacagcacctacag |
| | agtggtttctgtgctgacagtgctgcaccaggactggctgaatggcaaagagtacaagtg |
| | caaggtgtccaacaaggccctgcctgctcctattgagaaaaccatctccaaggccaaggg |
| | ccagccaagagaaccccaggtttacaccctgccacctagcagagaagagatgaccaagaa |
| | ccaggtgtccctgacctgcctggttaagggcttctacccctctgacattgctgtggaatg |
| | ggagagcaatggccagcctgaaaacaactacaagacaacccctcctgtgctggactctga |
| | tggctcattcttcctgtacagcaagctgactgtggacaagtccagatggcagcagggaaa |
| | tgtgttcagctgctctgtgatgcatgaggccctgcacaaccactacacccagaaaagtct |
| | gtctctgtcccctggcagaaagagaagaggctctggagaaggcagaggctccctgctgac |
| | atgtggggatgttgaagagaatcctgggcctatgtataggatgcaactgctcctcctgat |
| | tgctctgagcctggctcttgtgaccaactctgacatccagatgacacagagcccctccac |
| | actgtctgcatctgtgggagacagagtgaccatcacctgtagagccagccagtctatctc |
| | tagctggctggcctggtatcagcaaaagcctggcaaggcccctaagctgctgatctacaa |
| | ggccagcacacttgagtcaggggtgccctccagattttctggctctggatctggcacaga |
| | gttcaccctgaccatcagctccctgcagccagatgactttgccacctactactgccagca |
| | gtacaatacctactggacctttggccagggaacaaaggtggaaatcaagagaacagtggc |
| | tgccccatctgtgttcatcttcccaccatctgatgaacagctgaagtctggcactgcctc |
| | tgttgtgtgcctgctgaacaacttttaccccagagaagccaaggtgcagtggaaagtgga |
| | taatgccctgcagtctggcaatagccaagaatctgtgacagagcaggactccaaggattc |
| | cacctacagcctgagcagcaccctgacactgagcaaggctgactatgagaagcacaaagt |
| | gtatgcctgtgaagtgacacaccagggactgagcagcccagtgaccaagagcttcaacag |
| | gggagagtgctgataactcgaggacggggtgaactacgcctgaggatccgatctttttcc |
| | ctctgccaaaaattatggggacatcatgaagccccttgagcatctgacttctggctaata |
| | aaggaaatttattttcattgcaatagtgtgttggaattttttgtgtctctca |
| LMTP6.LANv2.T2A | aggctcagaggcacacaggagtttctgggctcaccctgcccccttccaacccctcagttc |
| (LMTP6.L02) | ccatcctccagcagctgtttgtgtgctgcctctgaagtccacactgaacaaacttcagcc |
| SEQ ID NO: 443 | tactcatgtccctaaaatgggcaaacattgcaagcagcaaacagcaaacacacagccctc |
| | cctgcctgctgaccttggagctggggcagaggtcagagacctctctgggcccatgccacc |
| | tccaacatccactcgaccccttggaatttcggtggagaggagcagaggttgtcctggcgt |
| | ggtttaggtagtgtgagagggccactacgggtttaggctgcccatgtaaggaggcaaggc |
| | ctggggacacccgagatgcctggttataattaacccagacatgtggctgccccccccccc |
| | cccaacacctgctgcctctaaaaataaccctgtccctggtggatcccactacgggtttag |
| | gctgcccatgtaaggaggcaaggcctggggacacccgagatgcctggttataattaaccc |
| | agacatgtggctgccccccccccccccaacacctgctgcctctaaaaataaccctgtccc |
| | tggtggatcccactacgggtttaggctgcccatgtaaggaggcaaggcctggggacaccc |
| | gagatgcctggttataattaacccagacatgtggctgccccccccccccccaacacctgc |
| | tgcctctaaaaataaccctgtccctggtggatcccctgcatgcgaagatcttcgaacaag |
| | gctgtgggggactgagggcaggctgtaacaggcttgggggccagggcttatacgtgcctg |
| | ggactcccaaagtattactgttccatgttcccggcgaagggccagctgtcccccgccagc |
| | tagactcagcacttagtttaggaaccagtgagcaagtcagcccttggggcagcccataca |
| | aggccatggggctgggcaagctgcacgcctgggtccggggtgggcacggtgcccgggcaa |
| | cgagctgaaagctcatctgctctcaggggcccctccctggggacagcccctcctggctag |
| | tcacaccctgtaggctcctctatataacccaggggcacaggggctgccctcattctacca |
| | ccacctccacagcacagacagacactcaggagccagccagcgtcgagatcttgctaccag |
| | tggaacagccactaaggattctgcagtgagagcagagggccagctaagtggtactctccc |
| | agagactgtctgactcacgccaccccctccaccttggacacaggacgctgtggtttctga |
| | gccaggtacagtgactcctttcggtaagtgcagtggaagctgtacactgcccaggcaaag |
| | cgtccgggcagcgtaggcgggcgactcagatcccagccagtggacttagcccctgtttgc |
| | tcctccgataactggggtgaccttggttaatattcaccagcagcctcccccgttgcccct |
| | ctggatccactgcttaaatacggacgaggacagggccctgtctcctcagcttcaggcacc |
| | accactgacctgggacagtcaggtaagtatcaaggttacaagacaggtttaaggagacca |
| | atagaaactgggcttgtcgagacagagaagactcttgcgtttctgataggcacctattgg |
| | tcttactgacatccactttgcctttctctccacaggttcgaattcgccgccaccatgtac |
| | agaatgcagctgctgctgctcattgccctgtctctggccctggtcaccaattctgaggtc |
| | cagctgcttgagagtggtggtggactggttcagcctggtggcagcctgagactgtcttgt |
| | gctgcctctggcttcaccttcagccactatatcatgatgtgggtcagacaggcccctggc |
| | aaaggcctggaatgggtgtcaggcatctacagcagtggtggcatcacagtgtatgctgac |
| | tctgtgaagggcagattcaccatcagcagagacaacagcaagaacaccctgtacctgcag |
| | atgaactccctgagagctgaggacacagcagtgtactactgtgcctatagaagaattggg |
| | gtgcccagaagggatgagtttgacatctggggccagggcaccatggttacagtgtcctct |
| | gccagcacaaagggcccctctgtttttccactggctcccagcagcaagagcaccagtggt |
| | ggaacagctgccctgggctgtctggtcaaggattacttccctgagcctgtgactgtgtcc |
| | tggaactctggggctctgacttctggggtgcacacctttccagctgtgctgcagtcctct |
| | ggcctgtactctctgtcctctgtggtcacagtgcctagctctagcctgggcacacagacc |
| | tacatctgcaatgtgaaccacaagcctagcaacaccaaggtggacaagagagtggaaccc |
| | aagagctgtgacaagacccacacctgtcctccatgtcctgctccagaactgcttggaggc |
| | ccttctgtgttcctgtttcctccaaagcctaaggacaccctgatgatcagcagaacccct |
| | gaagtgacctgtgtggtggttgatgtgtcccatgaggacccagaagtgaagttcaattgg |
| | tatgtggatggggttgaagtgcacaatgccaagaccaagcctagagaggaacagtacaac |
| | agcacctacagagtggtttctgtgctgacagtgctgcaccaggactggctgaatggcaaa |
| | gagtacaagtgcaaggtgtccaacaaggccctgcctgctcctattgagaaaaccatctcc |
| | aaggccaagggccagccaagagaaccccaggtttacaccctgccacctagcagagaagag |
| | atgaccaagaaccaggtgtccctgacctgcctggttaagggcttctacccctctgacatt |
| | gctgtggaatgggagagcaatggccagcctgaaaacaactacaagacaacccctcctgtg |
| | ctggactctgatggctcattcttcctgtacagcaagctgactgtggacaagtccagatgg |
| | cagcagggaaatgtgttcagctgctctgtgatgcatgaggccctgcacaaccactacacc |
| | cagaaaagtctgtctctgtcccctggcagaaagagaagaggctctggagaaggcagaggc |
| | tccctgctgacatgtggggatgttgaagagaatcctgggcctatgtataggatgcaactg |
| | ctcctcctgattgctctgagcctggctcttgtgaccaactctgacatccagatgacacag |
| | agcccctccacactgtctgcatctgtgggagacagagtgaccatcacctgtagagccagc |
| | cagtctatctctagctggctggcctggtatcagcaaaagcctggcaaggcccctaagctg |
| | ctgatctacaaggccagcacacttgagtcaggggtgccctccagattttctggctctgga |
| | tctggcacagagttcaccctgaccatcagctccctgcagccagatgactttgccacctac |
| | tactgccagcagtacaatacctactggacctttggccagggaacaaaggtggaaatcaag |
| | agaacagtggctgccccatctgtgttcatcttcccaccatctgatgaacagctgaagtct |
| | ggcactgcctctgttgtgtgcctgctgaacaacttttaccccagagaagccaaggtgcag |
| | tggaaagtggataatgccctgcagtctggcaatagccaagaatctgtgacagagcaggac |
| | tccaaggattccacctacagcctgagcagcaccctgacactgagcaaggctgactatgag |
| | aagcacaaagtgtatgcctgtgaagtgacacaccagggactgagcagcccagtgaccaag |
| | agcttcaacaggggagagtgctgataactcgaggacggggtgaactacgcctgaggatcc |
| | gatctttttccctctgccaaaaattatggggacatcatgaagccccttgagcatctgact |
| | tctggctaataaaggaaatttattttcattgcaatagtgtgttggaattttttgtgtctc |
| | tca |

HEK293 cells were plated at a density of 7.5×10⁵ cells/well in each well of a standard 6-well dish containing Dulbecco's modified eagle medium (DMEM) supplied with 10% fetal bovine serum (FBS). The next day, cells were transfected with CAG.L01 (SEQ ID NO: 435), CAG.L02 (SEQ ID NO: 437), and CAG.L03 (SEQ ID NO: 436) AAV constructs using Lifpofectamine 2000 (Invitrogen) according the manufacturer's protocol). Non-transfected cells were used as negative control. Cell culture medium was changed 24 hours post-transfection to opti-mem I reduced serum media (2 mL/well). Cell culture supernatant was harvested at 48 hours post-transfection, and cell lysates were harvested with RIPA buffer (Pierce) supplemented with EDTA-free protease inhibitor tablets (Pierce). Supernatant and lysates samples were stored at −80 C.

Proteins from supernatant or cell lysate samples were separated via the NuPAGE electrophoresis system (Thermo Fisher Scientific). For samples derived from cell lysates, 40 μg of protein was loaded unless indicated otherwise. Purified human IgG or Lanadelumab IgG (produced by Genscript) were used as loading controls (50-100 ng). Samples were heated with LDS sample buffer and NuPAGE reducing agent at 70 C for 10 minutes and then loaded into NuPAGE 4-12% Bis-Tris protein gels. Separated proteins were transferred to PVDF membranes using the iBlot2 dry blotting system according to manufacturer's instructions (P3 default setting was used for the protein transfer). Membranes were immediately washed in phosphate buffer saline with 0.1% v/v Tween-20 (PBST). Membranes were then incubated in blocking solution containing PBST and 1% Clear Milk Blocking Buffer (Thermo Scientific) for 1 hour at room temperature. Membranes were then incubated in fresh blocking solution supplemented with goat anti-human kappa light chain-HRP antibody (Bethyl Laboratories; 1:2000 dilution) and goat anti-human IgG Fc-HRP antibody (1:2000 dilution). Following antibody incubation, membranes were washed three times in PB ST for 5 minutes per wash. Finally, membranes were incubated in SuperSignal West Pico PLUS chemiluminescent substrate for 5 minutes and imaged on the BioRad Universal Hood II gel doc system for detection of horseradish peroxidase (HRP) signal.

### Results

Expression analysis of reporter transgene (eGFP) following transfection of different plasmid quantities (4 μg-nontransfected) showed a dose dependent increase in eGFP levels (FIG. 24B). Protein expression analysis of lanadelumab in the cell lysate (FIG. 24C) and in the cell supernatant (FIG. 24D) showed dose-dependent levels of lanadelumab in cell lysates and supernatant. Transfection with the construct containing the L02 transgene (SEQ ID NO: 286, CAG.L02 (SEQ ID NO: 437)), a codon-optimized and depleted of CpG dinucleotide sequences construction, resulted in higher expression levels compared to L01 transgene (SEQ ID NO: 141, CAG.L01 SEQ ID NO: 435). Transfection of CAG.L02 (SEQ ID NO: 437) and CAG.L03 (SEQ ID NO: 436) resulted in similar expression levels.

### 6.51 Example 51: Serum Expression of Lanadelumab in Mice

### Methods

A. Mouse experiments were performed with either AAV8 or AAV9 containing an AAV construct (as depicted in FIG. 24A) comprising the L01 sequence (SEQ ID NO: 141), which contains the Furin and F2A sequence (SEQ ID NO: 231). AAV8 and AAV9 vectors (n=5 mice per group; 2e11 genome copies (gc)) were administered to immunocompromised NSG mice via either intravenous (IV) or intramuscular (IM) routes. IV administrations were into the tail vein and IM administrations were bilateral into the gastrocnemius muscles. Mice injected with vehicle were included as controls. Seven weeks post administration mice were sacrificed and serum human antibody levels were determined by enzyme-linked immunosorbent assay (ELISA).

Lanadelumab levels in NSG mouse serum was assessed by ELISA. Briefly, mouse serum was obtained before treatment and at 1, 3, 5 and 7 weeks post in vivo gene transfection and stored at −80° C. 96-well plate was coated with 1 μg/ml human IgG-Fc fragment antibody (Bethyl, Montgomery, Tex.) in carbonate bicarbonate buffer (0.05M, pH 9.6, Sigma-Aldrich, St. Louis, Mo.) and incubated overnight at 4° C. After washing with Tween 20 washing buffer (PBST, 0.05%, Alfa Aesar, Haverhill, Mass.), plate was incubated with blocking buffer (3% BSA in PBS, ThermoFisher Scientific, Waltham, Mass.) for 1 h at 37° C. followed by washing. Mouse serum samples diluted in sample dilution buffer (0.1% Tween 20 and 3% BSA in PBS) was added to the plate (50 μl/well) and incubated for 2 h at 37° C. A standard curve of known lanadelumab concentrations ranging from 360 to 0.001 ng/mL was included in each plate. Plate was washed with PBST for five times after incubation. The levels of lanadelumab was detected by incubation with horseradish peroxidase-conjugated goat anti-human IgG (H+L) (200 ng/mL; Bethyl, Montgomery, Tex.) for 1 h at 37° C. The optical density was assessed using KPL TMB Microwell Peroxidase Substrate System (Seracare, Milford, Mass.) following the manufacturer's specifications. Data analysis was performed with SoftMax Pro version 7.0.2 software (Molecular Devices, Sunnyvale, Calif.).

### Results

A. Results from a representative experiment are shown in FIG. 25. Serum analysis of AAV8-, AAV9-injected and control (vehicle) NSG mice at 7 weeks post gene transfer showed expression and serum accumulation of Lanadelumab following AAV9 delivery (2E¹¹ gc). Serum Lanadelumab concentration was 100-fold higher in AAV9-injected mice compared to AAV8-injected mice and slightly higher in IV-AAV9-injected compared to IM-AAV9-injected mice. Serum human antibody levels in control mice were undetectable at 7 week time point.

B. In an analogous experiment, a time course of lanadelumab serum levels in NSG mice post-AAV9 administration (n=5 per group) was performed. AAV9 vectors (2E¹¹ gc) were injected either IV or IM (as above, in experiment A), and serum antibody levels were determined by ELISA at day 7 (D7), day 21 (D21), day 35 (D35), and day 49 (D49).

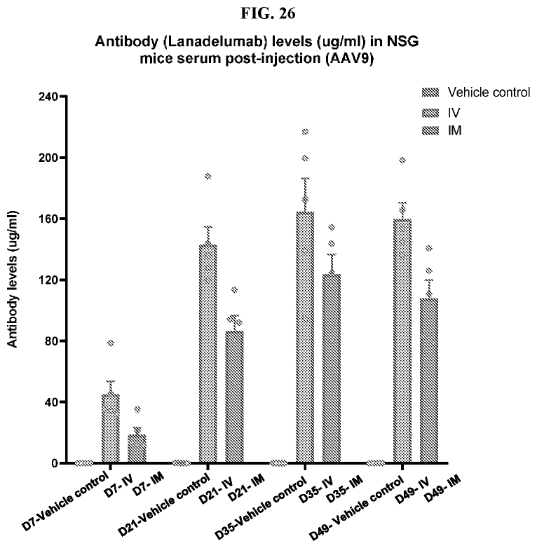

Serum Lanadelumab expression is detectable as early as 1 week (D7) after AAV9 administration in NSG mice. The expression levels increased at 3 weeks (D2), peaked at 5 weeks (D35) and then sustained up to 7 week post-injection (D49). It was observed that serum lanadelumab concentration is higher in IV vs. IM injected mice over the entire time course. See FIG. 26.

C. In an analogous experiment, a time course of lanadelumab serum levels in C/57BL6 mice post AAV8 administration was performed. The optimized expression cassette containing a liver-specific promoter and a codon optimized and CpG depleted transgene with a modified furin-2A processing signal resulted in robust serum antibody concentration when delivered intravenously using an AAV8 vector. Very high (>1 mg/ml) and sustained levels of functional anti-kallikrein antibody were achieved in the serum of C57BL/6 mice following IV vector administration at a dose of 1E¹³ gc/kg.

### 6.52 Example 52: Analysis of In Vitro Transduction and Expression of Tandem Liver- and Tandem Liver/Muscle-Specific Promoters Driving Expression of Lanadelumab

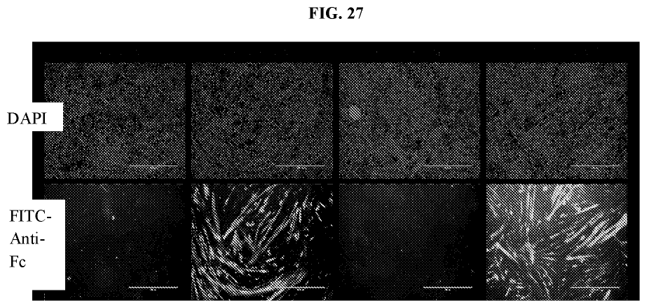

Cis plasmids expressing vectorized lanadelumab were packaged in AAV, then rAAV particles evaluated for potency of the transduction by AAV. Each cis plasmid contained lanadelumab (Mab 1) antibody light chain and heavy chain which are multicistrons driven by the CAG, ApoE.hAAT (SEQ ID NO: 412) or LMTP6 (SEQ ID NO: 320) promoter. Full-length lanadelumab antibody light chain and antibody heavy chain genes were separated by a furin 2A linker to ensure separate expression of each antibody chain. The entire cassette is flanked by AAV2 ITRs, and the genome is encapsidated in an AAV8 capsid for delivery to C2C12 cells (1E¹⁰ vg per well). For detection of antibody protein, following transduction, the cells are treated with FITC conjugated anti-Fc (IgG) antibody. The AAV8.CAG.Mab1 and AAV8.LMTP6.Mab1 infected cells show high expression in muscle cells, whereas the AAV8.hAAT.Mab1 infection does not result in expression of the antibody in muscle cells (FIG. 27). Cells appeared to be equally confluent and viable in all test wells, as seen by DAPI (DNA) staining (FIG. 27).

### 6.53 Example 53: Antibody Expression and Vector Biodistribution in Mouse Treated with AAV8 Lanadelumab Vectors Driven by Various Promoters

Thyroxine binding globulin (TBG) and alpha-1 antitrypsin (hAAT) promoters have been widely used as liver-specific promoters in previous pre-clinical and clinical gene therapy studies. A panel of designed promoter cassettes derived from multiple promoters and enhancers were generated and tested them in vitro by transfecting Huh7 cells, a human liver cell line. Promoter candidates were selected, which include ApoE.hAAT (SEQ ID NO: 412), LSPX1 (SEQ ID NO: 315), LSPX2 (SEQ ID NO: 316), LTP1 (SEQ ID NO: 317) and LMTP6 (SEQ ID NO: 320). AAV8 vectors encoding vectorized lanadelumab regulated by these promoter candidates were then generated. CAG (SEQ ID NO: 411) and TBG (SEQ ID NO: 423) promoters served as controls for ubiquitous and liver-specific promoters, respectfully. Strength of these promoters and vector biodistribution were tested in vivo by measuring lanadelumab protein expression compared to vector genome copy in each wild type mouse.

Vectors were administered intravenously to C57Bl/6 mice at equivalent doses (2.5×10¹² vg/kg) Mouse serum was collected biweekly, and lanadelumab protein expression levels were determined by ELISA. Liver samples were harvested at 49 days post vector administration. The presence of viral genomes in each sample was quantified using Lanadelumab probe and primer by Droplet Digital PCR (ddPCR) (the NAICA™ system from Stilla). The genome copy number of glucagon was also measured simultaneously in each sample, the viral genomes were then normalized and demonstrated as vector genome copy number per cell (assuming 2 glucagon/cell). Statistical analysis was performed using one-way ANOVA in GraphPad Prism 8.

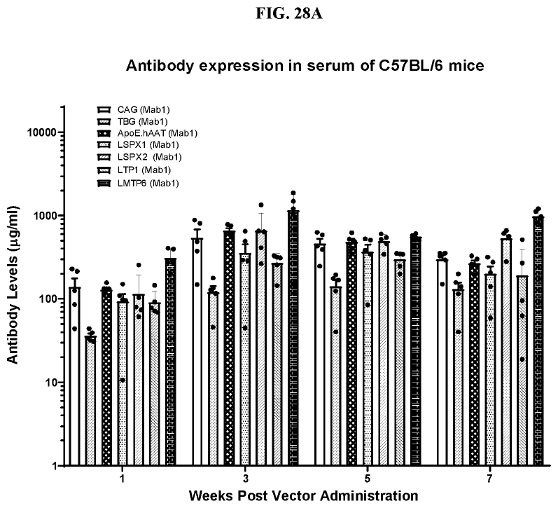

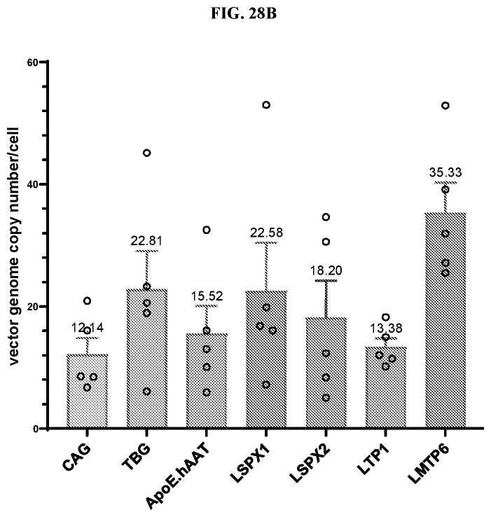

Among the AAV8 vectors with liver-specific promoters, the vectors driven by the ApoE.hAAT (SEQ ID NO: 412) and LMTP6 (SEQ ID NO: 320) promoters provided the highest amount of protein expression at all time points (FIG. 28A). While for the biodistribution data, there was no significant difference of vector genome copy number per cell in liver samples in animals treated with vectors driven by different promoters (FIG. 28B).

All liver-specific promoters outperform the TBG promoter (SEQ ID NO: 423), and the dual-specific LMTP6 promoter (SEQ ID NO: 320) consistently shows the highest expression in the serum (μg/ml) (FIG. 28).

### 6.54 Example 54: Lanadelumab Expression in Rat Serum Following Administration of Vectorized Antibody

A high level of Lanadelumab expression was detected in the serum of mice treated with AAV-Lanadelumab via IV administration. In parts of the study, the lanadelumab expression levels in different rat strains treated with different doses of AAV-Lanadelumab vectors and controls were examined.

### Experiment 1 (Wistar Rats):

To evaluate the route and the dose of vector administration in rats, a control vector AAV.CAG-LANv2.T2A (CAG.L02; SEQ ID NO: 437) was tested in Wistar rat. Eight to ten weeks old male Wistar rats were assigned into three groups (n=3 per group) to receive vector administration via IM or IV injection at a dose of 1×10¹³ vg/kg or 1×10¹⁴ vg/kg. Blood was collected at 7 days before treatment and 7, 10, 14, 17, 21, 28, 35, 42 and 49 days post vector administration and processed into serum.

TABLE 9
Study details for Lanadelumab expression in rat serum, Experiment 1.

| | Male 8-10 week old | | | Bleeds—Day sampled (X) | | | | | | | | | | |
|---|---|---|---|---|---|---|---|---|---|---|---|---|---|---|
| Rat | Wistar Rats (180-200 g) | | | Day | Day | Day | Day | Day | Day | Day | Day | Day | Day | Day |
| group | Treatment | N | ROA | −7 | 0 | 7 | 10 | 14 | 17 | 21 | 28 | 35 | 42 | 49 |
| 1 | AAV8.CAG.LANv2.T2A | 3 | IM | X | Administration | X | X | X | X | X | X | X | X | sacrifice |
| | Dose: 1e13 vg/kg | | | | | | | | | | | | | |
| 2 | AAV8.CAG.LANv2.T2A | 3 | IV | X | Administration | X | X | X | X | X | X | X | X | sacrifice |
| | Dose: 1e13 vg/kg | | | | | | | | | | | | | |
| 3 | AAV8.CAG.LANv2.T2A | 3 | IV | X | Administration | X | X | X | X | X | X | X | X | sacrifice |
| | Dose: 1e14 vg/kg | | | | | | | | | | | | | |

Levels of human IgG antibody in collected rat serum were detected by ELISA. Statistical analysis was done by one-way ANOVA with multiple comparisons at each time point using Prism

TABLE 10
Results of Lanadelumab expression in Wistar rats, Experiment 1

| | AAV8.CAG.Lanv2.T2A | | | AAV8.CAG.Lanv2.T2A | | | AAV8.CAG.Lanv2.T2A | | |
|---|---|---|---|---|---|---|---|---|---|
| Day | 1e13 vg/kg-IM | | | 1e13 vg/kg-IV | | | 1e14 vg/kg-IM | | |
| sampled | Mean | SEM | N | Mean | SEM | N | Mean | SEM | N |
| D7 | 4.1 | 0.96 | 3 | 8.43 | 0.64 | 3 | 33.6 | 13.86 | 3 |
| D10 | 7.67 | 1.91 | 3 | 12.3 | 0.81 | 3 | 64.5 | 30.2 | 3 |
| D14 | 9.37 | 0.73 | 3 | 23.13 | 0.18 | 3 | 123.23 | 69.12 | 3 |
| D17 | 4.97 | 2.23 | 3 | 92 | 53.52 | 3 | 245.17 | 151.09 | 3 |
| D21 | 2.04 | 0.9 | 3 | 33.06 | 5.84 | 3 | 252.63 | 149.41 | 3 |
| D28 | 92.08 | 87.07 | 3 | 65.23 | 41.69 | 3 | 117.97 | 112.47 | 3 |
| D35 | 14.46 | 12.31 | 3 | 88.1 | 56.68 | 3 | 122.97 | 74.94 | 3 |
| D42 | 82.29 | 80.46 | 3 | 40.43 | 26.77 | 3 | 108.02 | 99.26 | 3 |
| D49 | 1.66 | 0.95 | 3 | 81.4 | 39.96 | 3 | 216.30 | 118.18 | 3 |

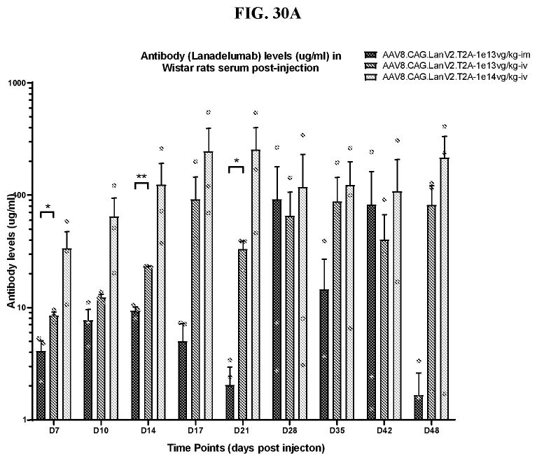

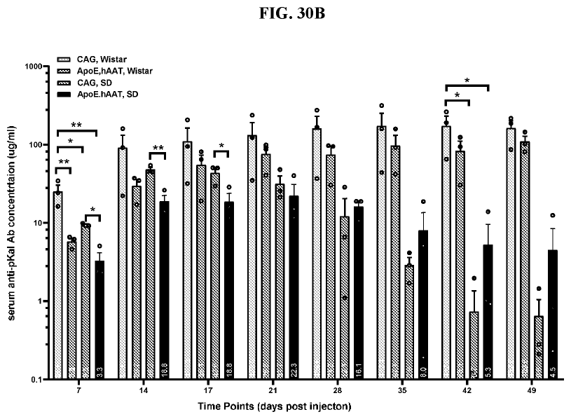

The levels of antibody in rat serum were detectable at 7 days post treatment. It increased over time and reached the peak level at 17 (lower dose) and 21 (higher dose) days post treatment in IV groups and 28 days in IM group. The antibody levels gradually decreased and sustains up to 48 days post treatment in all groups. For animals treated with lower dose (1×10¹³ vg/kg) vector, the antibody expression levels in IV groups are significantly higher than that in IM group at 7, 14 and 21 days post vector administration. For animals received IV administration, the antibody expression levels were dose-dependent at all time points. The highest level of lanadelumab expression was 252.6±149.4 μg/ml, which was detected in animals treated with higher dose (1×10¹⁴ vg/kg) at 21 days post IV administration. See FIG. 30A.

### Experiment 2 (Wistar and Sprague-Dawley Rats):

The aim of this experiment was to investigate the rat strain and the vector dose that will be used for a rat efficacy study. Eight to ten weeks old male Wistar and Sprague-Dawley (SD) rats were assigned into four groups (n=3 per group) to receive treatment of AAV8 vector carrying genome encoding lanadelumab driven by a universal promoter, CAG.L02 (SEQ ID NO: 437), or a liver-specific promoter, ApoE.hAAT.L02 (SEQ ID NO: 439). Vectors were administered via IV injection at a dose of 5×10¹³ vg/kg. Blood was collected at 7 days before treatment and 7, 10, 14, 17, 21, 28, 35, 42 and 49 days post vector administration and processed into the serum (Table 11). Levels of human IgG antibody in collected rat serum were detected by ELISA. Statistical analysis was done by one-way ANOVA with multiple comparisons at each time point using Prism.

TABLE 11
Study details for lanadelumab expression in rat serum, Experiment 2.

| | Male·8-10·week·old·¶ | | | | | | | | | | | | |
|---|---|---|---|---|---|---|---|---|---|---|---|---|---|
| | Wistar· or· Sprague· Dawley· (SD)· Rats | | | | | | | | | | | | |
| | (180-200g)¤ | | | → ·Bleeds·—·Day·sampled·(X)¤ | | | | | | | | | |
| Rat· | Treatment¶ | | | Day· | Day· | Day· | Day· | Day· | Day· | Day· | Day· | Day· | Day· |
| group¤ | Dose:·5e13·vg/kg¤ | N¤ | ROA¤ | −1¤ | 0¶¤ | 7¶·¤ | 14¤ | 17¤ | 21·¤ | 28¤ | 35·¤ | 42¤ | 49¤ |
| 1¤ | CAG.L02¶ | 3¤ | IV¤ | X¤ | Administration¤ | X¤ | X¤ | X¤ | X¤ | X¤ | X¤ | X¤ | sacrifice·¤ |
| | Wistar¤ | | | | | | | | | | | | |
| 2¤ | ApoE.hAAT.L02¶ | 3¤ | IV¤ | X¤ | Administration¤ | X¤ | X¤ | X¤ | X¤ | X¤ | X¤ | X¤ | sacrifice¤ |
| | Wistar¤ | | | | | | | | | | | | |
| 3¤ | CAG.L02¶ | 3¤ | IV¤ | X¤ | Administration¤ | X¤ | X¤ | X¤ | X¤ | X¤ | X¤ | X¤ | sacrifice¤ |
| | Sprague·Dawley·(SD)¤ | | | | | | | | | | | | |
| 4¤ | ApoE.hAAT.L02¶ | 3¤ | IV¤ | X¤ | Administration¤ | X¤ | X¤ | X¤ | X¤ | X¤ | X¤ | X¤ | sacrifice¤ |
| | Sprague·Dawley·(SD)¤ | | | | | | | | | | | | |

In this experiment, a control vector (CAG.L02, SEQ ID NO: 437) and vector ApoE.hAAT.L02 (SEQ ID NO: 439) were tested in Wistar and SD rats, respectively. Lanadelumab expression levels were higher in Wistar rat than SD rat in both vector groups at all time points. At the early time points, animals treated with control vector showed significant higher serum antibody levels than those treated with the liver-specific promoter containing vector. This was observed in Wistar rat at 7 days post treatment, and in SD rat at 7, 14 and 17 days post treatment. In Wistar rats, the concentrations of antibody gradually increased over time in both vectors group. The highest antibody levels were 173.1±78.8 mg/ml and 109.57±18.9 μg/ml at 35 and 49 days respectively in control CAG-Lanadelumab and hAAT-Lanadelumab vector-treated animals. In SD rats, however, the levels of antibody reached peaks at 14 and 21 days in control and lead vector-treated animals, respectively, and decreased gradually afterward in both groups. The highest antibody concentrations were 48.23±3.1 μg/ml and 22.33±8.98 μg/ml in CAG.L02 (SEQ ID NO: 437) and ApoE.hAAT.L02 (SEQ ID NO: 439) vector groups, respectively. See Table 12 and FIG. 30B.

TABLE 12
Results of Lanadelumab expression in Wistar rats, Experiment 2:

| | CAG-Wistar | | | ApoE.hAAT-Wistar | | |
|---|---|---|---|---|---|---|
| Day | 5e13 vg/kg-IV | | | 5e13 vg/kg-IV | | |
| sampled | Mean | SEM | N | Mean | SEM | N |
| D7 | 25.04 | 5.26 | 3 | 5.80 | 0.61 | 3 |
| D14 | 91.50 | 39.90 | 3 | 29.73 | 6.36 | 3 |
| D17 | 111.30 | 51.29 | 3 | 55.10 | 18.60 | 3 |
| D21 | 132.03 | 58.76 | 3 | 75.80 | 17.94 | 3 |
| D28 | 159.90 | 69.08 | 3 | 74.27 | 21.84 | 3 |
| D35 | 173.10 | 78.76 | 3 | 97.67 | 33.89 | 3 |
| D42 | 173.00 | 57.73 | 3 | 83.10 | 27.67 | 3 |
| D49 | 163.57 | 39.45 | 3 | 109.57 | 18.87 | 3 |

### 6.55 Example 55: Characterization of Vectorized Lanadelumab Regulated by Tissue-Specific Promoters Following Intramuscular Administration

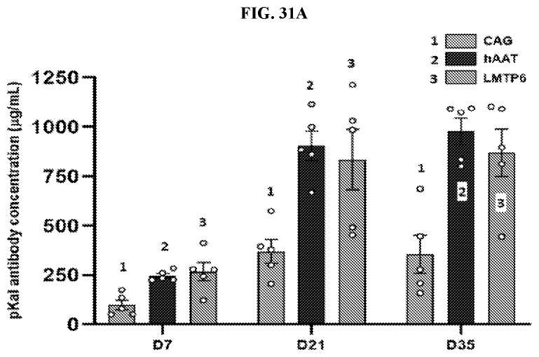

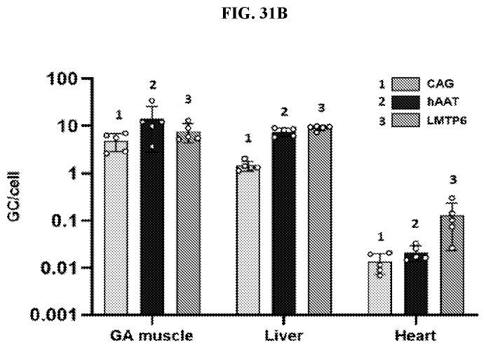

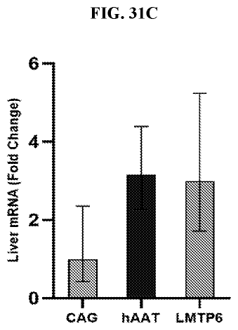

In a previous study, high liver-driven expression of vectorized lanadelumab with AAV8 regulated by the ApoE.hAAT or LMTP6 promoters was identified. The goal of this study was to characterize muscle-driven expression of the LMTP6 promoter following direct injection of lanadelumab vectors into the gastrocnemius (GA) muscle. Animals received bilateral injections of 5×10¹⁰ vg into the GA muscle. Serum was collected biweekly to measure systemic lanadelumab concentration (FIG. 31A). Animals were harvested at 49 days post-injection, and relevant tissues (liver, GA muscle, heart) were analyzed for vector biodistribution and transgene expression.

Vectors regulated by the hAAT and LMTP6 promoters demonstrated significantly increased antibody concentrations in serum compared to CAG at all time points (FIG. 31A). The hAAT and LMTP6 were not significantly different from each other in this experiment. Vector genome copies per cell of vectorized lanadelumab was detected and quantified in GA, liver and heart (FIG. 31B) with a notable difference of higher quantity of genome detected in heart for the dual muscle/liver promoter, LMTP6 vector. Increased liver RNA expression was also detected for all test vectors directly injected into GA muscle at 49 days (relative fold gene expression compared to a reference gene) (FIG. 31C). Gene expression (mRNA μg/mL) data from each of liver, GA muscle, and heart (FIG. 31D) indicates the dual specificity of LMPT6 in liver and muscle tissues following IM administration, whereas the hAAT-driven samples were reduced in muscle compared to LMTP6 and CAG. Significant differences were also seen between the hAAT and LMTP6 groups.

### 6.56 Example 56: Comparison of Lanadelumab Protein Levels in Mouse Serum Derived from Mice Treated with AAV-Lanadelumab Vectors Produced with Different Production Systems

Different AAV production protocols were developed to identify methods that can increase AAV titer and scalability, as well as assess the quality of vector product. Cis and trans plasmids to generate AAV8.Lanadelumab rAAV vectors (all having the same transgene driven by a CAG promoter) were constructed by well-known methods suitable for HEK293-transfected cell and also baculovirus (BV)/Sf9 insect cell production methods. Three different BV/Sf9 vector systems, BV1, BV2 and BV3, were provided as well as rAAV vector produced by an HEK293 method as a control. Purified rAAV product was injected into wild-type mice for this protein expression study (Table 13).

Young adult C57BL/6 mice (aged 8-10 weeks) were administered with above-mentioned vectors at 2.5 E¹² vg/kg via tail vein injection (n=5 per group). Serum was collected from each animal at 7, 21, 35, and 49 days post vector administration. Serum collected two days before injection (Day 0) served as baseline control. Levels of antibody (lanadelumab) expression were detected via ELISA. Data analysis was done by one-way ANOVA with multiple comparisons at each time point using Prism.

TABLE 13
Production system expression study design

| | AAV8.Lanadelumab | | | | | | | | | |
|---|---|---|---|---|---|---|---|---|---|---|
| | 5e10 vg (2.5e12 vg/kg) | | | | | | | | | |
| | rAAV | | | Bleeds sampled (X) | | | | | | |
| Mouse | Production | | | | Day 0 | Day 7 | Day 21 | Day 35 | Day 49 | |
| Group | method | N | ROA | Day −2 | (Week 0) | (Week 1) | (Week 3) | (Week 5) | (Week 7) | Day 50 |
| 1 | HEK cells | 5 | IV | X | Injection | X | X | X | X | Serum |
| 2 | BV1 | 5 | IV | X | Injection | X | X | X | X | Serum |
| Werum 3 | BV2 | 5 | IV | X | Injection | X | X | X | X | Serum |
| 4 | BV3 | 5 | IV | X | Injection | X | X | X | X | Serum |

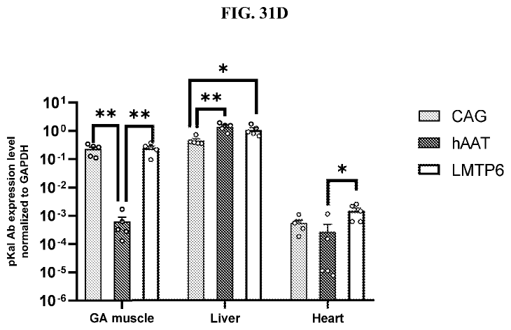

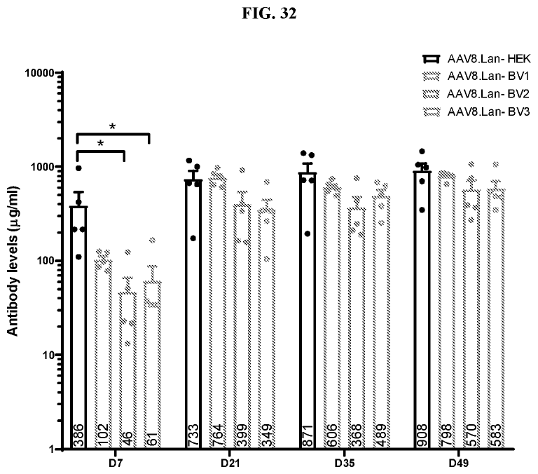

All production methods tested are viable based on this study, with greater yields from the HEK cell production method at the time points tested (see FIG. 32). Antibody expression in serum is detectable as early as 7 days post administration in all groups. The average of antibody concentration at Day 7 in the HEK production group is 386 μg/ml, which is significantly higher than other groups (61-102 μg/ml). The levels of antibody expression increase at day 21 by 1-, 6-, 7-, and 4-fold in BV1, BV2 and BV3 groups, respectively. Antibody expression levels sustained at 35 and 49 days post administration. There is no significant difference in between HEK produced vector and BV3 produced vectors at day 21, 35 and 49 time points.

### 6.57 Example 57: Vectorized Human Anti-pKal Antibody, Lanadelumab, Derived from Mouse Serum Suppressed Human pKal Function

In order to measure pKal function of lanadelumab derived from mouse serum following AAV-lanadelumab administration, we developed a fluorescence-based kinetic enzymatic functional assay. First, activated human plasma kallikrein (Enzyme Research Laboratories) was diluted in sample dilution buffer (SDB; 1×PBS, 3% BSA, 0.1% Tween-20) to top concentration of 100 nM. This pKal was two-fold serially diluted for a total of 12 concentrations in the dilution series (100 nM-0.05 nM). From each dilution, and in duplicate, 254, was placed in one well of a 96-well, opaque flat-bottomed plate along with 254, of SDB. Then, 50 μL of the fluorogenic substrate Pro-Phe-Arg-7-Amino-4-Methylcoumarin (PFR-AMC) (Bachem) prepared at 100 μM in assay buffer (50 mM Tris, 250 mM NaCl, pH 7.5) was added to each well. The samples were immediately run in kinetic mode for AMC fluorescence at excitation/emission wavelengths of 380/460 nm, respectively, for 3 hours using a SpectraMax 3 fluorescent plate reader.

The signal-to-noise ratio for each pKal concentration RFU (last RFU fluorescent value chosen) was calculated by dividing its RFU by background PFR-AMC substrate fluorescence. The two lowest pKal concentrations with a signal-to-noise ratio ≥2 (6.25 nM and 12.5 nM) were then chosen to evaluate the suppressive effect and range of lanadelumab antibody of pKal function in a lanadelumab dose response. Lanadelumab (GenScript) or human IgG control antibody was diluted in SDB to top concentration of 200 nM and two-fold serially diluted to 0.39 nM. Next, 25 μL pKal (each of two chosen concentrations) was incubated with 25 μL lanadelumab or human IgG at 30° C. for 1 hour. Antibody-pKal mixture was then given PFR-AMC and immediately run in kinetic mode for AMC fluorescence at excitation/emission wavelengths of 380/460 nm, respectively, for 3 hours using a SpectraMax fluorescent plate reader.

In vitro pKal functional assay. When used, mouse serum was diluted in sample dilution buffer and incubated 1:1 with 6.25 nM (1.56 nM in-well) pKal for 30° C./1 hour. For total IgG purification from mouse serum, antibody was purified using the Protein A Spin Antibody Purification Kit (BioVision) according to manufacturer's protocol. Total antibody concentration was measured using a Nanodrop spectrophotometer, with OD absorbance=280 nM. AMC standard curve was generated by a two-fold downward dilution series of AMC (500 nM, eleven dilutions and blank subtracted) diluted in assay buffer. AMC was read as end point fluorescence at excitation/emission wavelengths of 380/460 nm, respectively. Specific plasma kallikrein activity was calculated as: (adjusted experimental sample Vmax, RFU/sec)×(Conversion factor, AMC standard curve μM/RFU)/(pKal concentration, nM). Percent reduction in pKal activity was derived from calculating day 49 by day −7 pKal activity.

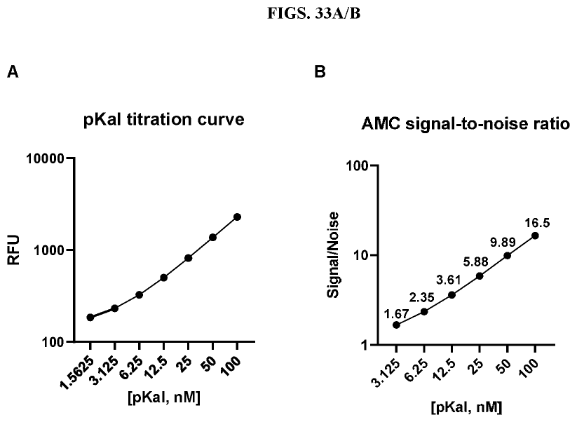

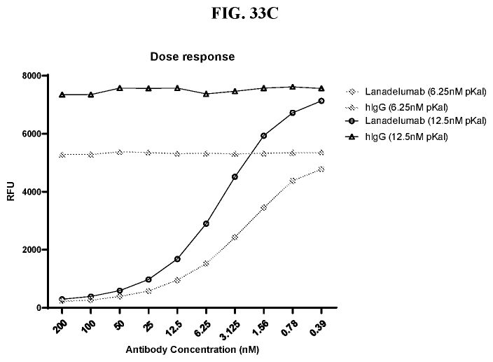

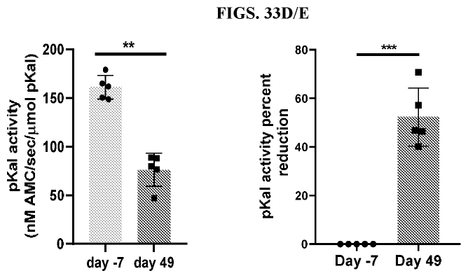

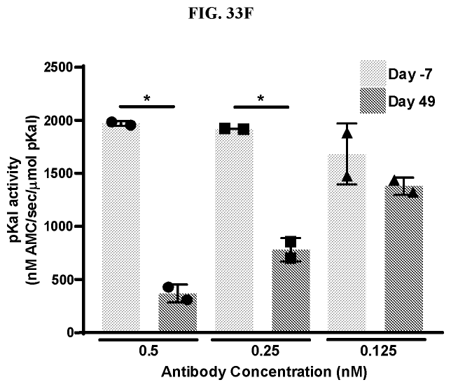

To determine whether AAV-derived lanadelumab can suppress plasma kallikrein function, we developed the in vitro AMC substrate-based functional assay to address this in a proof-of-concept study (FIGS. 33A and 33B). In this assay, antibody-containing medium is incubated with activated human pKal, as described above. Antibody-bound pKal is then given the synthetic peptide substrate Pro-Phe-Arg conjugated to AMC (PFR-AMC) and amount of released AMC is measured over time at excitation/emission wavelengths of 380/460 nm, respectively, for 3 hours. The assay showed noticeable lanadelumab-mediated suppression of pKal activity down to 0.1 nM (in-well concentration) (FIG. 33C) at a defined enzyme concentration. We first sought to determine whether serum from mice administered lanadelumab-encoded AAV could suppress pKal activity. Serum from mice 49 days post-administration was diluted 1:25 (in range predicted to be suppressive), incubated with pKal in vitro, and pKal activity was assayed. Serum from mice post-vector administration, as opposed to 7 days pre-administration, suppressed pKal activity, as reflected in a significant reduction of enzyme activity and a ˜50% percent reduction in pKal activity between the two time points (FIGS. 33D and 33E).

Further experiments show that suppression was due to the lanadelumab within the serum. Reasoning that the human IgG, namely lanadelumab, would only be found in the day 49 post-administration IgG fraction, but not the day −7 pre-administration samples, purified and total IgG antibody was used from the aforementioned day −7 and day 49 mouse serum samples to test pKal suppression. Indeed, only lanadelumab-containing purified IgG from day 49 post-administration serum, but not IgG from the pre-administration time point, suppressed human pKal function (FIG. 33F).

### 6.58 Example 58: Effects of AAV-Lanadelumab in Carrageenan Animal Models

### 6.58.1 Example 58A: Effects of AAV-Lanadelumab in the Carrageenan Paw Edema Model in Mice

Inflammation models induced by carrageenan are frequently used acute inflammation models. Carrageenan (Cg) is a strong chemical agent that functions in stimulating the release of inflammatory and proinflammatory mediators, including bradykinin, histamine, tachykinins, reactive oxygen, and nitrogen species. Typical signs of inflammation include edema, hyperalgesia, and erythema, which develop immediately following the treatment of carrageenan. This example evaluated the effect of AAV-mediated gene delivery of Lanadelumab on carrageenan-induced paw edema in mice.

In total eighty young adult (8-9 weeks old) male C57BL/6 mice were used for this study. Animals were divided into eight groups as listed in Table 14. Paw edema was induced by a single subcutaneous (s.c.) injection of 30 μL of 0.7% or 1% carrageenan solution. Test vectors and positive control Diclofenac were administered at 21 days and 30 minutes prior to carrageenan treatment. Blood was collected before vectors injection and at 7 and 21 days post injection from mice in groups 1, 3, 4, 5, 7 and 8. Paw volume was measured using a digital Plethysmometer prior to carrageenan injection, and at 2, 4, 6, 8, 24 and 48 hours after injection. All animals were sacrificed 48 hours after carrageenan injection. Liver and paw specimens were also collected at the necropsy.

TABLE 14
Carrageenan (Cg) Paw Edema Mouse Study design

| | | Test Items | Route, Dose | Pre- | |
|---|---|---|---|---|---|
| *Gr. | Group/treatment | Dose level | volume | treatment time | N |
| 1 | Cg (0.7%)/ | 0 | I.P., 10 mL/kg | 30 min | 10 |
| | Vehicle of | | | before Cg | |
| | diclofenac | | | | |
| 2 | Cg ( 0.7%)/ | 60 mg/kg | I.P., 10 mL/kg | 30 min | 10 |
| | Diclofenac | | | before Cg | |
| 3 | Cg ( 0.7%)/ | 1e13 vg/kg | I.V., | 3 weeks | 10 |
| | Vector 1 | | 3.333 mL/kg | before Cg | |
| 4 | Cg (0.7%)/ | 1e13 vg/kg | I.V., | 3 weeks | 10 |
| | AAV8. | | 3.333 mL/kg | before Cg | |
| | ApoE.hAAT.Lan | | | | |
| 5 | Cg (1.0%)/ | 0 | I.P., 10 mL/kg | 30 min | 10 |
| | Vehicle of | | | before Cg | |
| | diclofenac | | | | |
| 6 | Cg (1.0%)/ | 60 mg/kg | I.P., 10 mL/kg | 30 min | 10 |
| | Diclofenac | | | before Cg | |
| 7 | Cg (1.0%)/ | 1e13 vg/kg | I.V., | 3 weeks | 10 |
| | Vector 1 | | 3.333 mL/kg | before Cg | |
| 8 | Cg (1.0%)/ | 1e13 vg/kg | I.V., | 3 weeks | 10 |
| | AAV8. | | 3.333 mL/kg | before Cg | |
| | ApoE.hAAT.Lan | | | | |

Vector 1:AAV8-GFP

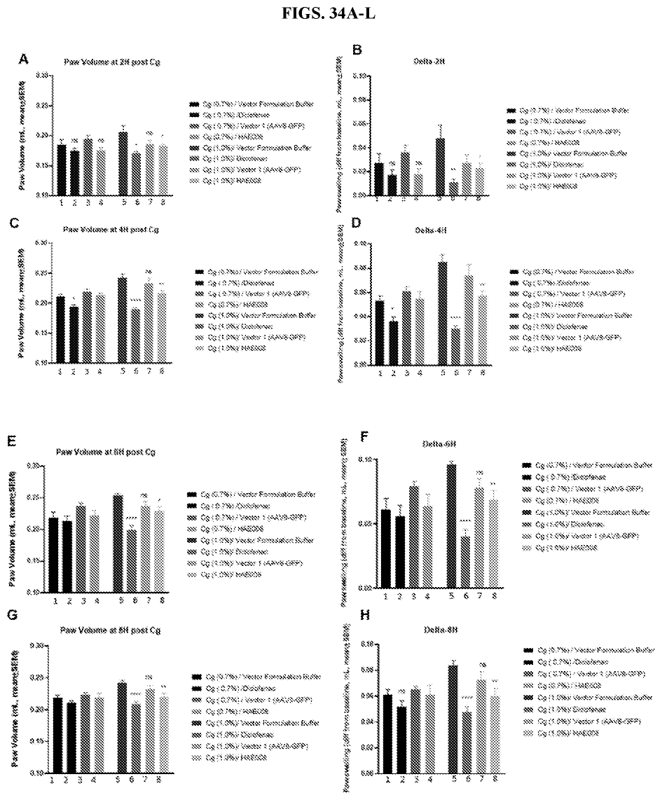

Both 0.7% and 1.0% carrageenan induced swelling in the injected paw; however, swelling was more pronounced with 1.0% carrageenan injection (FIGS. 34, A-L). In the positive control groups (Group 2 and 6), diclofenac treatment significantly decreased the paw volume at 2, 4, 6, 8 and 24 hours post carrageenan injection in 1.0% Cg model (group 2), while a significant decrease on paw volume was observed only at 4 and 24 hours post injection in 0.7% Cg model (group 6).

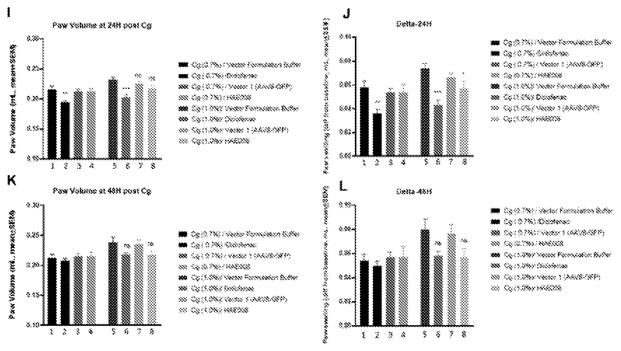

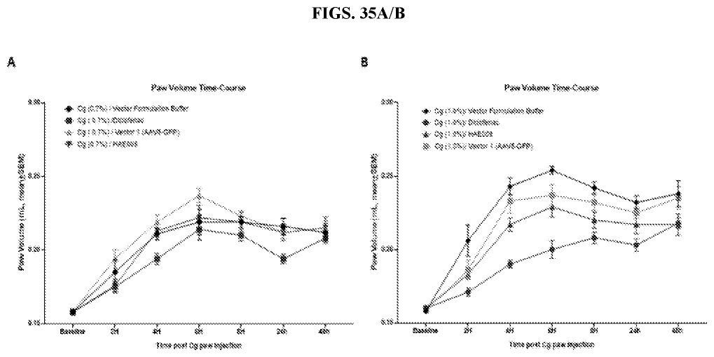

ApoE.hAAT.L02 (SEQ ID NO: 439) treatment significantly reduced the paw volume at 2, 4, 6 and 8 hours post carrageenan injection in 1,0% Cg model when compared with the vehicle control group (group 1, vector formulation buffer) (FIGS. 35A and 35B). However, no effect of ApoE.hAAT.L02 treatment was observed in 0.7% Cg model at any time points (FIGS. 35A and 35 B). There is no significant difference in between groups treated with vehicle (groups 1 and 4) or control vector (AAV-GFP, groups 3 and 7) in both 1.0% and 0.7% Cg models (FIGS. 33A-L). All data was analyzed with One-way ANOVA with Dunnett's post-hoc test for multiple comparisons.

These data indicate that acute inflammation can be successfully induced in mouse paw with a single subcutaneous injection of 1% carrageenan solution. Lanadelumab, a human IgG antibody produced in mouse serum via AAV-mediated gene delivery significantly reduces the severity of inflammation in mouse 1% carrageenan model.

### 6.58.2 Example 58B: Efficacy Study of AAV-Lanadelumab on Carrageenan-Induced Paw Edema in Wistar Rats

This project aims to evaluate the efficacy of AAV-mediated antibody (Lanadelumab) therapy on Carrageenan-induced paw edema in Wistar rats. The experiment involves 50 male Wistar rats in total, divided into 5 groups (see Table. 15). Paw edema will be induced by a single subcutaneous (s.c.) injection of 100 μL of 1% carrageenan solution. Test articles will be administered at different time points (30 minutes, 21 days, 24 hours, or 1 hour) prior to carrageenan treatment.

TABLE 15
Grouping and Treatment for Wistar Rat Study

| | | | | | | | Dosage | |
|---|---|---|---|---|---|---|---|---|
| Group | N | Carrageenan | Treatment | R.O.A | Dosing Time | Conc. | mL/kg | mg/kg/vg/kg |
| 1 | 10 | Yes | Vehicle^{a} | IV | 30 min prior to | N/A | 1.6 | N/A |
| | | | | | Carrageenan injection | | | |
| 2 | 10 | Yes | ApoE.hAAT.L02 | IV | 21 days prior to | 6.25E+13 | 1.6 | 1e14 |
| | | | | | Carrageenan injection | vg/mL | | vg/kg |
| 3 | 10 | Yes | Control AAV | IV | 21 days prior to | 6.25E+13 | 1.6 | 1e14 |
| | | | (irrelevant transgene) | | Carrageenan injection | vg/ml | | vg/kg |
| 4 | 10 | Yes | Lanadelumab | SC | 24 hours prior to | 4.81 | 6.237 | 30 |
| | | | | | Carrageenan injection | mg/ml | | mg/kg |
| 5 | 10 | Yes | Dexamethasone^{b} | PO | 1 hour prior to | 1 | 10 | 10 |
| | | | | | and 6 hours after | mg/ml | | mg/kg |
| | | | | | Carrageenan injection | | | |

^{a}Formulation buffer
^{b}Normal saline

Body weight will be measured twice a week. On 7 days before vectors administration, and 7 and 14 days post-vector injection, 0.3 mL of blood will be collected by retroorbital/submandibular bleeding in all rats, and then processed into serum. Paw volume will be measured prior to carrageenan injection, and at 2, 4, 6, 8, 24 hours post carrageenan injection. All animals will be sacrificed 24 hours after carrageenan injection. At necropsy, blood will be collected for serum preparation by intracardiac puncture. Then, three pieces of liver (approximately 50 mg per unit) from the same lobe of each animal will be collected and cryopreserved in three separate tubes, respectively. The right paw from all rats will be resected, formalin-fixed, and embedded in paraffin (FFPE). See Table 16.

TABLE 16
Measurement and Sampling Regime for Wistar Rat Study

| | | | | | | | | Paw edema measurement | | | | | | Bleed and |
|---|---|---|---|---|---|---|---|---|---|---|---|---|---|---|
| | Bleed | Treatment | Bleed | Bleed | Treatment | Treatment | Carageenan | (hr post carrageenan) | | | | | | end of study |
| Group | Day −7 | Day 0 | Day 7 | Day 14 | Day 20 | Day 21 | Day 21 | 0 h | 2 h | 4 h | 6 h | 8 h | 24 h | Day 22 |
| 1 | √ | x | √ | √ | x | Vehicle | √ | √ | √ | √ | √ | √ | √ | √ |
| 2 | √ | AAV8.Lan | √ | √ | x | x | √ | √ | √ | √ | √ | √ | √ | √ |
| 3 | √ | Control AAV | √ | √ | x | x | √ | √ | √ | √ | √ | √ | √ | √ |

Data will be presented as mean±SEM. Statistical analysis including t-test or ANOVA will be performed using Graphpad, prism 6.0 with the significance level alpha of 0.05.

### 6.59 Example 59: AAV-Mediated Anti-Plasma Kallikrein Antibody Treatment for Diabetic Retinopathy

Diabetic retinopathy (DR) and diabetic macular edema (DME) are the most common complications of diabetes and the leading causes of blindness among working-age adults. Chronic hyperglycemia leads to retinal microvasculature damage and promote an increase in microvascular permeability, upregulation of inflammatory response, and fluid accumulation into the neural retina, which ultimately result in visual loss. Intravitreal administration of anti-VEGFs is the first-line treatment for DME. However, DME response to anti-VEGF is highly variable. Only 54% of DME patients treated with consecutive, monthly anti-VEGF gain vision (Elman M J, Aiello L P, Beck R W, et al. Randomized trial evaluating ranibizumab plus prompt or deferred laser or triamcinolone plus prompt laser for diabetic macular edema. Ophthalmology 2010; 117:1064-1077; Brown D M, Nguyen Q D, Marcus D M, et al. Long-term outcomes of ranibizumab therapy for diabetic macular edema: The 36-month results from two phase III trials: RISE and RIDE. Ophthalmology 2013; 120:2013-2022). Recent studies suggested that plasma kallikrein, a mediator of vascular leakage and inflammation, may play an important role in the pathogenesis of DR and DME independent of VEGF (Gao -B-B, Clermont A, Rook S, et al. Extracellular carbonic anhydrase mediates hemorrhagic retinal and cerebral vascular permeability through prekallikrein activation. Nature Medicine 2007; 13:181; Clermont A, Chilcote T J, Kita T, et al. Plasma kallikrein mediates retinal vascular dysfunction and induces retinal thickening in diabetic rats. Diabetes 2011; 60:1590-1598; and Kita T, Clermont A C, Murugesan N, et al. Plasma kallikrein-kinin system as a VEGF-independent mediator of diabetic macular edema. Diabetes 2015; 64:3588-3599). Plasma kallikrein inhibitors could be the potential alternative therapies for DR and DME patients who are not response to anti-VEGF therapies. This study aims to test the possibility of therapy for DR/DME using ocular vectorized antibody delivery with adeno-associated virus (AAV) gene transfer.

Vectorized anti-plasma kallikrein antibody (anti-pKal Ab such as Lanadelumab) sequence has been constructed and tested for its use in treatment of hereditary angioedema (HAE), as described above. In this study AAV8.CAG.LAN Ab and AAV8.CAG.GFP will be used as test vectors. AAV8.NUL will serve as a control vector.

First, the transduction efficiency and cell type specificity is evaluated in wild type rats. Adult Wistar rats (3-4 months old) will be used for this study. Vectors including AAV8.CAG.LAN Ab, AAV8.CAG.GFP and AAV8.NUL, will be delivered in rat eyes via subretinal (SR) injection at different doses (5×10⁸ vg/eye and 5×10⁹ vg/eye) in 3 μl of formulation buffer. Fundus and OCT imaging will be performed at 2, 4 and 8 weeks after SR injection. Ocular tissue specimens will be collected at 8 weeks post administration. Levels of antibody expression in individual ocular tissues including retina and retinal pigment epithelium (RPE) will be quantified by ELISA. Cell type specificity will be determined by immunofluorescent staining with various retinal cell markers.

Streptozotocin (STZ)-induced diabetic Wistar rats will be used as the animal model for efficacy studies. Diabetes will be induced by intravenous injection of STZ at the dose of 60 mg/kg of the body weight in young adult Wistar rats (8-10 weeks old). Significant and progressive loss of visual acuity generally starts at 8 weeks following induction in this model. AAV8.CAG.pKal Ab vector will be injected subretinally at 4 or 8 weeks after the induction of Diabetes. Contralateral eye will be injected with AAV.NUL vector and serve as a control. Fundus, fluorescein angiography (FA) and OCT imaging, electroretinography (ERG) and Optokinetic nystagmus (OKN) will be tested at 8, 12 and 16 weeks following induction of Diabetes. Ocular tissues or the whole eyeballs will be collected at 16-17 weeks post Diabetes induction. Antibody expression in ocular tissues will be quantified by ELISA. Retina structure changes and neuron survival will be evaluated by histology and immunofluorescent staining.

### 6.60 Example 60: Characterization of Tissue-Restricted Transgene Immunogenicity

The goal of this study is to understand transgene immunogenicity and/or tolerance induction in the context of ubiquitous, tissue-specific, or tandem promoters. Hypothesis: Vectors driven by liver-specific and liver-muscle tandem promoters will demonstrate reduced immunogenicity compared to vectors driven by a ubiquitous promoter. To test this hypothesis, four AAV vectors that drive expression of a highly immunogenic membrane-bound ovalbumin (mOVA) were constructed. These vectors differ in their promoter sequences which includes: a) a ubiquitous CAG promoter (SEQ ID NO: 411) b) the liver-specific hAAT promoter with upstream ApoE enhancer (SEQ ID NO: 412) c) the muscle-specific CK8 promoter cassette composed of the CK core promoter and three copies of a modified MCK enhancer (SEQ ID NO: 413) and d) liver-muscle tandem promoter 6 (LMTP6, SEQ ID NO: 320) that contains sequence elements derived from hAAT and CK8. Initial experiments will measure the immune response following intravenous (IV) vector administration within mice. Study endpoints will include characterization of humoral and cell-mediated immune responses against the mOVA transgene product. In addition, tissues will be harvested for vector biodistribution and transgene expression analysis.

### 6.61 Example 61: Plasma Expression of Vectorized Lanadelumab in Cynomolgus Monkeys

Plasma kinetics of lanadelumab expression in non-human primates administered AAV vectors encoding lanadelumab antibodies will be assessed. The goal is to assess and select the dose of AAV8.ApoE.hAAT.Lan vector that results in sustained lanadelumab expression of at least 200 μg/ml lanadelumab by three months or more. The cynomolgus monkey is chosen as the test system because of its established usefulness and acceptance as a model for AAV biodistribution studies in a large animal species and for further translation to human. All animals on this study are naïve with respect to prior treatment.

Nine cynomolgus animals will be used. Animals judged suitable for experimentation based on clinical sign data and prescreening antibody titers will be placed in three study groups, each receiving a different dosage of AAV vector, by body weight using computer-generated random numbers. Each set of three animals will be administered a single i.v. dose of the vector AAV8.ApoE.hAAT.Lan vector (described above) at the dose of 1e12 gc/kg (Group 1), 1e13 gc/kg (Group 2), and 1e14 gc/kg (Group 3).

Clinical signs will be recorded at least once daily beginning approximately two weeks prior to initiation of dosing and continuing throughout the study period. The animals will be observed for signs of clinical effects, illness, and/or death. Additional observations may be recorded based upon the condition of the animal at the discretion of the Study Director and/or technicians.

Blood samples will be collected from a peripheral vein for bioanalytical analysis prior to dose administration and then at weekly intervals for at least 3 months (9 weeks). The samples will be collected in clot tubes and the times recorded. The tubes will be maintained at room temperature until fully clotted, then centrifuged at approximately 2400 rpm at room temperature for 15 minutes. The serum will be harvested, placed in labeled vials, frozen in liquid nitrogen, and stored at −60° C. or below.

A gross necropsy will be performed on any animal found dead or sacrificed moribund, and at the end of the study period, at least three months after vector administration. All animals, except those found dead, will be sedated with 8 mg/kg of ketamine HCl IM, maintained on an isoflurane/oxygen mixture and provided with an intravenous bolus of heparin sodium, 200 IU/kg. The animals will be perfused via the left cardiac ventricle with 0.001% sodium nitrite in saline. Animals found dead will be necropsied but will not be perfused.

As primary endpoint analysis, plasma samples will be assayed for lanadelumab concentration by ELISA and/or western blot, to be reported at least as μg lanadelumab per ml plasma; and lanadelumab activity, for example, kallikrein inhibition, by fluorogenic assay.

The presence of antibodies against lanadelumab (ADAs) in the serum will be evaluated by ELISA and lanadelumab binding assays. Biodistribution of the vector and lanadelumab coding transcripts will be assessed in necroscopy samples by quantitative PCR and NGS methods. Tissues to be assayed include liver, muscle, and heart. Toxicity assessment will be done by full pathology, including assaying liver enzymes, urinalysis, cardiovascular health, and more.

### 6.62 Example 62: Vectorized Adalimumab IgG and Fab Cassettes: Design and Characterization

An AAV transgene cassette was constructed (SEQ ID NO: 451) that drives ubiquitous expression of vectorized adalimumab IgG (TNF001, SEQ ID NO: 452). The protein coding sequence is composed of the heavy and light chains of adalimumab separated by a Furin cleavage site, Gly-Ser-Gly (GSG) linker (SEQ ID NO: 427), and T2A self-processing peptide sequence (SEQ ID NO: 429). The specific sequence configuration yields expression of separate heavy and light chain peptides. The entire reading frame is codon-optimized and depleted of CpG dinucleotides. Expression is driven by the CAG promoter (SEQ ID NO: 411). Similarly, an additional cassette was developed (SEQ ID NO: 453) that drives expression of a Fab containing the adalimumab variable regions (TNF002, SEQ ID NO: 454). Constructs are outlined in FIG. 1, and FIG. 12A and sequences are provided in Table. 17.

TABLE 17

| Name/ | |
|---|---|
| SEQ ID NO. | Sequence |
| TNF001 | gacattgattattgactagttattaatagtaatcaattacggggtcattagttcatagcc |
| SEQ ID NO: 451 | catatatggagttccgcgttacataacttacggtaaatggcccgcctggctgaccgccca |
| | acgacccccgcccattgacgtcaataatgacgtatgttcccatagtaacgccaataggga |
| | ctttccattgacgtcaatgggtggagtatttacggtaaactgcccacttggcagtacatc |
| | aagtgtatcatatgccaagtacgccccctattgacgtcaatgacggtaaatggcccgcct |
| | ggcattatgcccagtacatgaccttatgggactttcctacttggcagtacatctacgtat |
| | tagtcatcgctattaccatggtcgaggtgagccccacgttctgcttcactctccccatct |
| | cccccccctccccacccccaattttgtatttatttattttttaattattttgtgcagcga |
| | tgggggcggggggggggggggggcgcgcgccaggcggggcggggcggggcgaggggcggg |
| | gcggggcgaggcggagaggtgcggcggcagccaatcagagcggcgcgctccgaaagtttc |
| | cttttatggcgaggcggcggcggcggcggccctataaaaagcgaagcgcgcggcgggcgg |
| | gagtcgctgcgcgctgccttcgccccgtgccccgctccgccgccgcctcgcgccgcccgc |
| | cccggctctgactgaccgcgttactcccacaggtgagcgggcgggacggcccttctcctc |
| | cgggctgtaattagcgcttggtttaatgacggcttgtttcttttctgtggctgcgtgaaa |
| | gccttgaggggctccgggagggccctttgtgcggggggagcggctcggggggtgcgtgcg |
| | tgtgtgtgtgcgtggggagcgccgcgtgcggctccgcgctgcccggcggctgtgagcgct |
| | gcgggcgcggcgcggggctttgtgcgctccgcagtgtgcgcgaggggagcgcggccgggg |
| | gcggtgccccgcggtgcggggggggctgcgaggggaacaaaggctgcgtgcggggtgtgt |
| | gcgtgggggggtgagcagggggtgtgggcgcgtcggtcgggctgcaaccccccctgcacc |
| | cccctccccgagttgctgagcacggcccggcttcgggtgcggggctccgtacggggcgtg |
| | gcgcggggctcgccgtgccgggcggggggtggcggcaggtgggggtgccgggcggggcgg |
| | ggccgcctcgggccggggagggctcgggggaggggcgcggcggcccccggagcgccggcg |
| | gctgtcgaggcgcggcgagccgcagccattgccttttatggtaatcgtgcgagagggcgc |
| | agggacttcctttgtcccaaatctgtgcggagccgaaatctgggaggcgccgccgcaccc |
| | cctctagcgggcgcggggcgaagcggtgcggcgccggcaggaaggaaatgggcggggagg |
| | gccttcgtgcgtcgccgcgccgccgtccccttctccctctccagcctcggggctgtccgc |
| | ggggggacggctgccttcgggggggacggggcagggcggggttcggcttctggcgtgtga |
| | ccggcggctctagagcctctgctaaccatgttcatgccttcttctttttcctacagctcc |
| | tgggcaacgtgctggttattgtgctgtctcatcattttggcaaagaattcgccaccatgt |
| | acagaatgcagctgctgctgctcattgccctgtctctggccctggtcaccaattctgaag |
| | tgcagctggtggaaagtggtggtggactggtgcagcctggcagaagcctgagactgtctt |
| | gtgctgcctctggcttcacctttgatgactatgccatgcactgggtcagacaggcccctg |
| | gcaaaggactggaatgggtgtcagccatcacctggaactctggccacattgactatgctg |
| | actctgtggaaggcagattcaccatcagcagagacaatgccaagaacagcctgtacctgc |
| | agatgaactccctgagagctgaggacacagcagtgtactactgtgccaaggtgtcctacc |
| | tgagcacagccagcagcctggattattggggccagggcacactggttacagtgtcctctg |
| | ccagcacaaagggcccctctgtttttccactggctcccagcagcaagagcaccagtggtg |
| | gaacagctgccctgggctgtctggtcaaggattacttccctgagcctgtgacagtgtctt |
| | ggaactcaggggctctgacctctggggtgcacacatttccagctgtgctgcagtcctctg |
| | gcctgtactctctgtcctctgtggtcacagtgcctagctctagcctgggcacccagacct |
| | acatctgcaatgtgaaccacaagcctagcaacaccaaggtggacaagaaggtggaaccca |
| | agagctgtgacaagacccacacctgtcctccatgtcctgctccagaactgcttggaggcc |
| | cttctgtgttcctgtttcctccaaagcctaaggacaccctgatgatcagcagaacccctg |
| | aagtgacctgtgtggtggttgatgtgtcccatgaggacccagaagtgaagttcaattggt |
| | atgtggatggggttgaagtgcacaatgctaagaccaagcctagagaggaacagtacaaca |
| | gcacctacagagtggtgtctgtgctgacagtgctgcatcaggactggctgaatggcaaag |
| | agtacaagtgcaaagtgtccaacaaggccctgcctgctcctattgagaaaaccatctcca |
| | aggccaagggccagccaagagaaccccaggtttacacactgccacctagcagagatgagc |
| | tgaccaagaaccaggtgtccctgacctgcctggttaagggcttctacccctctgacattg |
| | ctgtggaatgggagagcaatggccagcctgagaacaactacaagacaacccctcctgtgc |
| | tggactctgatggctcattcttcctgtacagcaagctgactgtggacaagtccagatggc |
| | agcaggggaatgtgttcagctgctctgtgatgcatgaggccctgcacaaccactacaccc |
| | agaaaagtctgagtctgagccctggcaagagaaagagaagaggctctggagaaggcagag |
| | gctccctgctgacatgtggggatgttgaagagaatcctgggcctatgtataggatgcaac |
| | tgctcctcctgattgctctgagcctggctcttgtgaccaactctgacatccagatgacac |
| | agagccctagcagcctgtctgcttctgtgggagacagagtgaccatcacatgcagagcca |
| | gccagggaatcagaaactacctggcctggtatcagcaaaagcctggcaaggcccctaagc |
| | tgctgatctatgcagccagcacactgcagtcaggggtgccaagcagattttcaggctctg |
| | gctctggcacagacttcaccctgaccatttctagcctgcagcctgaggatgtggccacct |
| | actactgccagagatacaacagagccccatacacctttggacagggcacaaaggtggaaa |
| | tcaagagaacagtggctgccccatctgtgttcatcttcccaccatctgatgaacagctga |
| | agtctggcactgcctctgttgtgtgcctgctgaacaacttctaccctagagaagccaagg |
| | tgcagtggaaggttgacaatgccctgcagtctggcaatagccaagaatctgtgacagagc |
| | aggactccaaggattccacctacagcctgagcagcaccctgacactgagcaaggctgact |
| | atgagaagcacaaagtgtatgcctgtgaagtgacacaccagggactgagcagcccagtga |
| | ccaagagcttcaacaggggagagtgctgataactcgaggacggggtgaactacgcctgag |
| | gatccgatctttttccctctgccaaaaattatggggacatcatgaagccccttgagcatc |
| | tgacttctggctaataaaggaaatttattttcattgcaatagtgtgttggaattttttgt |
| | gtctctcactcg |
| Adalimumab | atgtacagaatgcagctgctgctgctcattgccctgtctctggccctggtcaccaattct |
| Full-length mAb | gaagtgcagctggtggaaagtggtggtggactggtgcagcctggcagaagcctgagactg |
| single chain | tcttgtgctgcctctggcttcacctttgatgactatgccatgcactgggtcagacaggcc |
| construct | cctggcaaaggactggaatgggtgtcagccatcacctggaactctggccacattgactat |
| transgene | gctgactctgtggaaggcagattcaccatcagcagagacaatgccaagaacagcctgtac |
| (TNF001) | ctgcagatgaactccctgagagctgaggacacagcagtgtactactgtgccaaggtgtcc |
| (HC-Furin- | tacctgagcacagccagcagcctggattattggggccagggcacactggttacagtgtcc |
| linker-T2A-LC) | tctgccagcacaaagggcccctctgtttttccactggctcccagcagcaagagcaccagt |
| SEQ ID NO: 452 | ggtggaacagctgccctgggctgtctggtcaaggattacttccctgagcctgtgacagtg |
| | tcttggaactcaggggctctgacctctggggtgcacacatttccagctgtgctgcagtcc |
| | tctggcctgtactctctgtcctctgtggtcacagtgcctagctctagcctgggcacccag |
| | acctacatctgcaatgtgaaccacaagcctagcaacaccaaggtggacaagaaggtggaa |
| | cccaagagctgtgacaagacccacacctgtcctccatgtcctgctccagaactgcttgga |
| | ggcccttctgtgttcctgtttcctccaaagcctaaggacaccctgatgatcagcagaacc |
| | cctgaagtgacctgtgtggtggttgatgtgtcccatgaggacccagaagtgaagttcaat |
| | tggtatgtggatggggttgaagtgcacaatgctaagaccaagcctagagaggaacagtac |
| | aacagcacctacagagtggtgtctgtgctgacagtgctgcatcaggactggctgaatggc |
| | aaagagtacaagtgcaaagtgtccaacaaggccctgcctgctcctattgagaaaaccatc |
| | tccaaggccaagggccagccaagagaaccccaggtttacacactgccacctagcagagat |
| | gagctgaccaagaaccaggtgtccctgacctgcctggttaagggcttctacccctctgac |
| | attgctgtggaatgggagagcaatggccagcctgagaacaactacaagacaacccctcct |
| | gtgctggactctgatggctcattcttcctgtacagcaagctgactgtggacaagtccaga |
| | tggcagcaggggaatgtgttcagctgctctgtgatgcatgaggccctgcacaaccactac |
| | acccagaaaagtctgagtctgagccctggcaagagaaagagaagaggctctggagaaggc |
| | agaggctccctgctgacatgtggggatgttgaagagaatcctgggcctatgtataggatg |
| | caactgctcctcctgattgctctgagcctggctcttgtgaccaactctgacatccagatg |
| | acacagagccctagcagcctgtctgcttctgtgggagacagagtgaccatcacatgcaga |
| | gccagccagggaatcagaaactacctggcctggtatcagcaaaagcctggcaaggcccct |
| | aagctgctgatctatgcagccagcacactgcagtcaggggtgccaagcagattttcaggc |
| | tctggctctggcacagacttcaccctgaccatttctagcctgcagcctgaggatgtggcc |
| | acctactactgccagagatacaacagagccccatacacctttggacagggcacaaaggtg |
| | gaaatcaagagaacagtggctgccccatctgtgttcatcttcccaccatctgatgaacag |
| | ctgaagtctggcactgcctctgttgtgtgcctgctgaacaacttctaccctagagaagcc |
| | aaggtgcagtggaaggttgacaatgccctgcagtctggcaatagccaagaatctgtgaca |
| | gagcaggactccaaggattccacctacagcctgagcagcaccctgacactgagcaaggct |
| | gactatgagaagcacaaagtgtatgcctgtgaagtgacacaccagggactgagcagccca |
| | gtgaccaagagcttcaacaggggagagtgctga |
| TNF002 | gacattgattattgactagttattaatagtaatcaattacggggtcattagttcatagcc |
| SEQ ID NO: 453 | catatatggagttccgcgttacataacttacggtaaatggcccgcctggctgaccgccca |
| | acgacccccgcccattgacgtcaataatgacgtatgttcccatagtaacgccaataggga |
| | ctttccattgacgtcaatgggtggagtatttacggtaaactgcccacttggcagtacatc |
| | aagtgtatcatatgccaagtacgccccctattgacgtcaatgacggtaaatggcccgcct |
| | ggcattatgcccagtacatgaccttatgggactttcctacttggcagtacatctacgtat |
| | tagtcatcgctattaccatggtcgaggtgagccccacgttctgcttcactctccccatct |
| | cccccccctccccacccccaattttgtatttatttattttttaattattttgtgcagcga |
| | tgggggcggggggggggggggggcgcgcgccaggcggggcggggcggggcgaggggcggg |
| | gcggggcgaggcggagaggtgcggcggcagccaatcagagcggcgcgctccgaaagtttc |
| | cttttatggcgaggcggcggcggcggcggccctataaaaagcgaagcgcgcggcgggcgg |
| | gagtcgctgcgcgctgccttcgccccgtgccccgctccgccgccgcctcgcgccgcccgc |
| | cccggctctgactgaccgcgttactcccacaggtgagcgggcgggacggcccttctcctc |
| | cgggctgtaattagcgcttggtttaatgacggcttgtttcttttctgtggctgcgtgaaa |
| | gccttgaggggctccgggagggccctttgtgcggggggagcggctcggggggtgcgtgcg |
| | tgtgtgtgtgcgtggggagcgccgcgtgcggctccgcgctgcccggcggctgtgagcgct |
| | gcgggcgcggcgcggggctttgtgcgctccgcagtgtgcgcgaggggagcgcggccgggg |
| | gcggtgccccgcggtgcggggggggctgcgaggggaacaaaggctgcgtgcggggtgtgt |
| | gcgtgggggggtgagcagggggtgtgggcgcgtcggtcgggctgcaaccccccctgcacc |
| | cccctccccgagttgctgagcacggcccggcttcgggtgcggggctccgtacggggcgtg |
| | gcgcggggctcgccgtgccgggcggggggtggcggcaggtgggggtgccgggcggggcgg |
| | ggccgcctcgggccggggagggctcgggggaggggcgcggcggcccccggagcgccggcg |
| | gctgtcgaggcgcggcgagccgcagccattgccttttatggtaatcgtgcgagagggcgc |
| | agggacttcctttgtcccaaatctgtgcggagccgaaatctgggaggcgccgccgcaccc |
| | cctctagcgggcgcggggcgaagcggtgcggcgccggcaggaaggaaatgggcggggagg |
| | gccttcgtgcgtcgccgcgccgccgtccccttctccctctccagcctcggggctgtccgc |
| | ggggggacggctgccttcgggggggacggggcagggcggggttcggcttctggcgtgtga |
| | ccggcggctctagagcctctgctaaccatgttcatgccttcttctttttcctacagctcc |
| | tgggcaacgtgctggttattgtgctgtctcatcattttggcaaagaattcgccaccatgt |
| | acagaatgcagctgctgctgctcattgccctgtctctggccctggtcaccaattctgaag |
| | tgcagctggtggaaagtggtggtggactggtgcagcctggcagaagcctgagactgtctt |
| | gtgctgcctctggcttcacctttgatgactatgccatgcactgggtcagacaggcccctg |
| | gcaaaggactggaatgggtgtcagccatcacctggaactctggccacattgactatgctg |
| | actctgtggaaggcagattcaccatcagcagagacaatgccaagaacagcctgtacctgc |
| | agatgaactccctgagagctgaggacacagcagtgtactactgtgccaaggtgtcctacc |
| | tgagcacagccagcagcctggattattggggccagggcacactggttacagtgtcctctg |
| | ccagcacaaagggcccctctgtttttccactggctcccagcagcaagagcaccagtggtg |
| | gaacagctgccctgggctgtctggtcaaggattacttccctgagcctgtgacagtgtctt |
| | ggaactcaggggctctgacctctggggtgcacacatttccagctgtgctgcagtcctctg |
| | gcctgtactctctgtcctctgtggtcacagtgcctagctctagcctgggcacccagacct |
| | acatctgcaatgtgaaccacaagcctagcaacaccaaggtggacaagaaggtggaaccca |
| | agagctgtgacaagacccacagaaagagaagaggctctggagaaggcagaggctccctgc |
| | tgacatgtggggatgttgaagagaatcctgggcctatgtataggatgcaactgctcctcc |
| | tgattgctctgagcctggctcttgtgaccaactctgacatccagatgacacagagcccta |
| | gcagcctgtctgcttctgtgggagacagagtgaccatcacatgcagagccagccagggca |
| | tcagaaactacctggcctggtatcagcagaagccaggcaaggcccctaagctgctgatct |
| | atgcagccagcacactgcagagtggggtgccaagcagattttcaggctctggctctggca |
| | cagacttcaccctgaccatttctagcctgcagcctgaggatgtggccacctactactgcc |
| | agagatacaacagagccccatacacctttggacagggcacaaaggtggaaatcaagagaa |
| | cagttgcagcaccctcagttttcatcttccccccctcagatgaacagctgaagtctggca |
| | ctgcctctgttgtgtgcctgctgaacaacttctaccccagagaagccaaggtgcagtgga |
| | aggttgacaatgccctgcagtcaggcaacagccaagaatctgtgactgaacaggattcca |
| | aggatagcacctacagcctgagcagcaccctgacactgagcaaggctgactatgagaagc |
| | acaaagtgtatgcctgtgaagtgacccaccagggactgagcagcccagtgaccaagagct |
| | tcaacaggggagagtgctgataactcgaggacggggtgaactacgcctgaggatccgatc |
| | tttttccctctgccaaaaattatggggacatcatgaagccccttgagcatctgacttctg |
| | gctaataaaggaaatttattttcattgcaatagtgtgttggaattttttgtgtctctcac |
| | tcg |
| Adalimumab | atgtacagaatgcagctgctgctgctcattgccctgtctctggccctggtcaccaattct |
| Fab single chain | gaagtgcagctggtggaaagtggtggtggactggtgcagcctggcagaagcctgagactg |
| construct | tcttgtgctgcctctggcttcacctttgatgactatgccatgcactgggtcagacaggcc |
| transgene | cctggcaaaggactggaatgggtgtcagccatcacctggaactctggccacattgactat |
| (TNF002) | gctgactctgtggaaggcagattcaccatcagcagagacaatgccaagaacagcctgtac |
| (HC-Furin- | ctgcagatgaactccctgagagctgaggacacagcagtgtactactgtgccaaggtgtcc |
| linker-T2A-LC) | tacctgagcacagccagcagcctggattattggggccagggcacactggttacagtgtcc |
| SEQ ID NO: 453 | tctgccagcacaaagggcccctctgtttttccactggctcccagcagcaagagcaccagt |
| | ggtggaacagctgccctgggctgtctggtcaaggattacttccctgagcctgtgacagtg |
| | tcttggaactcaggggctctgacctctggggtgcacacatttccagctgtgctgcagtcc |
| | tctggcctgtactctctgtcctctgtggtcacagtgcctagctctagcctgggcacccag |
| | acctacatctgcaatgtgaaccacaagcctagcaacaccaaggtggacaagaaggtggaa |
| | cccaagagctgtgacaagacccacagaaagagaagaggctctggagaaggcagaggctcc |
| | ctgctgacatgtggggatgttgaagagaatcctgggcctatgtataggatgcaactgctc |
| | ctcctgattgctctgagcctggctcttgtgaccaactctgacatccagatgacacagagc |
| | cctagcagcctgtctgcttctgtgggagacagagtgaccatcacatgcagagccagccag |
| | ggcatcagaaactacctggcctggtatcagcagaagccaggcaaggcccctaagctgctg |
| | atctatgcagccagcacactgcagagtggggtgccaagcagattttcaggctctggctct |
| | ggcacagacttcaccctgaccatttctagcctgcagcctgaggatgtggccacctactac |
| | tgccagagatacaacagagccccatacacctttggacagggcacaaaggtggaaatcaag |
| | agaacagttgcagcaccctcagttttcatcttccccccctcagatgaacagctgaagtct |
| | ggcactgcctctgttgtgtgcctgctgaacaacttctaccccagagaagccaaggtgcag |
| | tggaaggttgacaatgccctgcagtcaggcaacagccaagaatctgtgactgaacaggat |
| | tccaaggatagcacctacagcctgagcagcaccctgacactgagcaaggctgactatgag |
| | aagcacaaagtgtatgcctgtgaagtgacccaccagggactgagcagcccagtgaccaag |
| | agcttcaacaggggagagtgctga |
| Adalimumab | gaagtgcagctggtggaaagtggtggtggactggtgcagcctggcagaagcctgagactg |
| Heavy Chain | tcttgtgctgcctctggcttcacctttgatgactatgccatgcactgggtcagacaggcc |
| (HC) IgG1 | cctggcaaaggactggaatgggtgtcagccatcacctggaactctggccacattgactat |
| SEQ ID NO: 444 | gctgactctgtggaaggcagattcaccatcagcagagacaatgccaagaacagcctgtac |
| | ctgcagatgaactccctgagagctgaggacacagcagtgtactactgtgccaaggtgtcc |
| | tacctgagcacagccagcagcctggattattggggccagggcacactggttacagtgtcc |
| | tctgccagcacaaagggcccctctgtttttccactggctcccagcagcaagagcaccagt |
| | ggtggaacagctgccctgggctgtctggtcaaggattacttccctgagcctgtgacagtg |
| | tcttggaactcaggggctctgacctctggggtgcacacatttccagctgtgctgcagtcc |
| | tctggcctgtactctctgtcctctgtggtcacagtgcctagctctagcctgggcacccag |
| | acctacatctgcaatgtgaaccacaagcctagcaacaccaaggtggacaagaaggtggaa |
| | cccaagagctgtgacaagacccacacctgtcctccatgtcctgctccagaactgcttgga |
| | ggcccttctgtgttcctgtttcctccaaagcctaaggacaccctgatgatcagcagaacc |
| | cctgaagtgacctgtgtggtggttgatgtgtcccatgaggacccagaagtgaagttcaat |
| | tggtatgtggatggggttgaagtgcacaatgctaagaccaagcctagagaggaacagtac |
| | aacagcacctacagagtggtgtctgtgctgacagtgctgcatcaggactggctgaatggc |
| | aaagagtacaagtgcaaagtgtccaacaaggccctgcctgctcctattgagaaaaccatc |
| | tccaaggccaagggccagccaagagaaccccaggtttacacactgccacctagcagagat |
| | gagctgaccaagaaccaggtgtccctgacctgcctggttaagggcttctacccctctgac |
| | attgctgtggaatgggagagcaatggccagcctgagaacaactacaagacaacccctcct |
| | gtgctggactctgatggctcattcttcctgtacagcaagctgactgtggacaagtccaga |
| | tggcagcaggggaatgtgttcagctgctctgtgatgcatgaggccctgcacaaccactac |
| | acccagaaaagtctgagtctgagccctggcaag |
| | IgG1 CH1 and CH2 are shown bold; hinge region is shown in |
| | italic. |
| Adalimumab VH | Gaagtgcagctggtggaaagtggtggtggactggtgcagcctggcagaagcctgagactg |
| SEQ ID NO: 445 | tcttgtgctgcctctggcttcacctttgatgactatgccatgcactgggtcagacaggcc |
| | cctggcaaaggactggaatgggtgtcagccatcacctggaactctggccacattgactat |
| | gctgactctgtggaaggcagattcaccatcagcagagacaatgccaagaacagcctgtac |
| | ctgcagatgaactccctgagagctgaggacacagcagtgtactactgtgccaaggtgtcc |
| | tacctgagcacagccagcagcctggattattggggccagggcacactggttacagtgtcc |
| | tct |
| | CDRs are underlined. |
| IgG1 CH1 | gccagcacaaagggcccctctgtttttccactggctcccagcagcaagagcaccagtggt |
| SEQ ID NO: 446 | ggaacagctgccctgggctgtctggtcaaggattacttccctgagcctgtgacagtgtct |
| | tggaactcaggggctctgacctctggggtgcacacatttccagctgtgctgcagtcctct |
| | ggcctgtactctctgtcctctgtggtcacagtgcctagctctagcctgggcacccagacc |
| | tacatctgcaatgtgaaccacaagcctagcaacaccaaggtggacaagaaggtg |
| IgG1 CH2 | gctccagaactgcttggaggcccttctgtgttcctgtttcctccaaagcctaaggacacc |
| SEQ ID NO: 447 | ctgatgatcagcagaacccctgaagtgacctgtgtggtggttgatgtgtcccatgaggac |
| | ccagaagtgaagttcaattggtatgtggatggggttgaagtgcacaatgctaagaccaag |
| | cctagagaggaacagtacaacagcacctacagagtggtgtctgtgctgacagtgctgcat |
| | caggactggctgaatggcaaagagtacaagtgcaaagtgtccaacaaggccctgcctgct |
| | cctattgagaaaaccatctccaaggccaagggccagccaagagaaccccaggtttacaca |
| | ctgccacctagcagagatgagctgaccaagaaccaggtgtccctgacctgcctggttaag |
| | ggcttctacccctctgacattgctgtggaatgggagagcaatggccagcctgagaacaac |
| | tacaagacaacccctcctgtgctggactctgatggctcattcttcctgtacagcaagctg |
| | actgtggacaagtccagatggcagcaggggaatgtgttcagctgctctgtgatgcatgag |
| | gccctgcacaaccactacacccagaaaagtctgagtctgagccctggcaag |
| Adalimumab | gctccagaactgcttggaggcccttctgtgttcctgtttcctccaaagcctaaggacacc |
| Light Chain | ctgatgatcagcagaacccctgaagtgacctgtgtggtggttgatgtgtcccatgaggac |
| (kappa) | ccagaagtgaagttcaattggtatgtggatggggttgaagtgcacaatgctaagaccaag |
| SEQ ID NO: 448 | cctagagaggaacagtacaacagcacctacagagtggtgtctgtgctgacagtgctgcat |
| | caggactggctgaatggcaaagagtacaagtgcaaagtgtccaacaaggccctgcctgct |
| | cctattgagaaaaccatctccaaggccaagggccagccaagagaaccccaggtttacaca |
| | ctgccacctagcagagatgagctgaccaagaaccaggtgtccctgacctgcctggttaag |
| | ggcttctacccctctgacattgctgtggaatgggagagcaatggccagcctgagaacaac |
| | tacaagacaacccctcctgtgctggactctgatggctcattcttcctgtacagcaagctg |
| | actgtggacaagtccagatggcagcaggggaatgtgttcagctgctctgtgatgcatgag |
| | gccctgcacaaccactacacccagaaaagtctgagtctgagccctggcaag |
| Adalimumab VL | Gacatccagatgacacagagccctagcagcctgtctgcttctgtgggagacagagtgacc |
| SEQ ID NO: 449 | atcacatgcagagccagccagggaatcagaaactacctggcctggtatcagcaaaagcct |
| | ggcaaggcccctaagctgctgatctatgcagccagcacactgcagtcaggggtgccaagc |
| | agattttcaggctctggctctggcacagacttcaccctgaccatttctagcctgcagcct |
| | gaggatgtggccacctactactgccagagatacaacagagccccatacacctttggacag |
| | ggcacaaaggtggaaatcaag |
| | CDRs are underlined. |
| Adalimumab | agaacagttgcagcaccctcagttttcatcttccccccctcagatgaacagctgaagtct |
| Light chain C- | ggcactgcctctgttgtgtgcctgctgaacaacttctaccccagagaagccaaggtgcag |
| Domain (CL) | tggaaggttgacaatgccctgcagtcaggcaacagccaagaatctgtgactgaacaggat |
| SEQ ID NO: 450 | tccaaggatagcacctacagcctgagcagcaccctgacactgagcaaggctgactatgag |
| | aagcacaaagtgtatgcctgtgaagtgacccaccagggactgagcagcccagtgaccaag |
| | agcttcaacaggggagagtgctga |

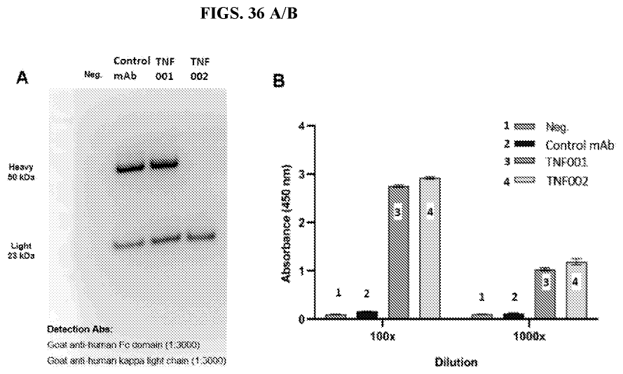

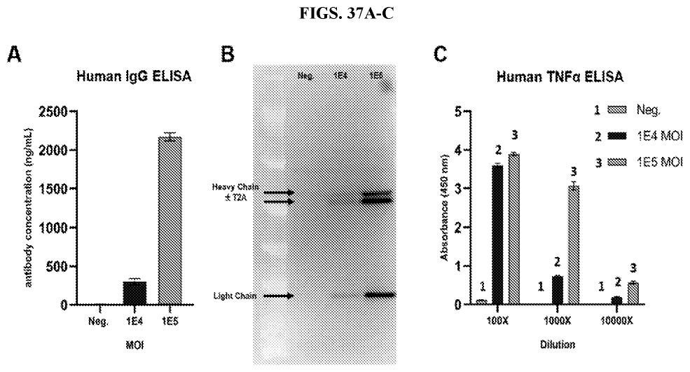

Plasmid expression was characterized via western blot following transfection into 293T cells (FIG. 36A). In addition, plasmid-expressed antibody activity was assessed via ELISA for binding of recombinant human TNFα (FIG. 36B). In addition, this plasmid was used to produce recombinant AAV8 vectors. Expression and activity of adalimumab produced from AAV8 was evaluated with the aforementioned assays (FIGS. 37A-37C).

### 6.63 Example 63: Self-Complementary Adalimumab Fab Transgene Cassettes: Design and Characterization

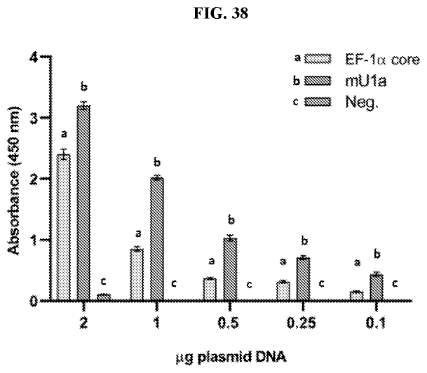

Two self-complementary AAV (scAAV) transgene cassettes encoding vectorized adalimumab Fab were generated. The transgenes are driven by the ubiquitous mU1a (SEQ ID NO: 414) or EF-1α (SEQ ID NO: 415) core promoters. These plasmids were compared for Fab expression via transfection into 293T cells (FIG. 38). The mU1a-driven vector displayed a higher absorbance values suggesting higher Fab concentration within the cell supernatant.

### 6.64 Example 64: TNFα Binding Across Model Species with Vectorized Adalimumab IgG and Fab

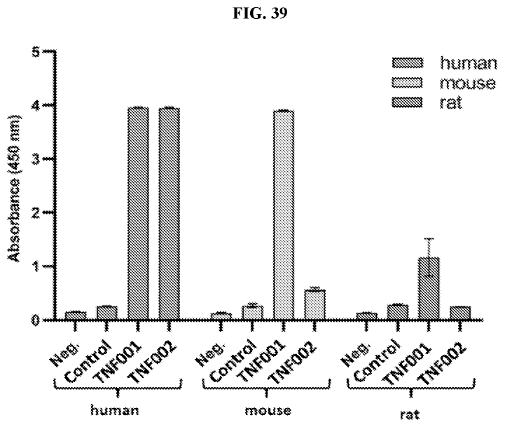

Vectorized adalimumab candidates for TNFα isolated from model species including human, mouse, and rat were tested for their binding capacity. Vectorized antibodies were expressed and secreted into cell supernatant following cis plasmid transfection into 293T cells. The cell supernatant was tested in an ELISA where the plates were coated with recombinant TNFα derived from the aforementioned species (FIG. 39). Adalimumab IgG effectively bound human and mouse derived TNFα. The Fab demonstrates a similar binding profile to human TNFα as the IgG. However, the Fab displays poor binding to mouse TNFα compared to adalimumab IgG. Both IgG and Fab display far reduced binding to rat TNFα.

### 6.65 Example 65: AAV-Mediated Ocular Gene Therapy for Non-Infectious Posterior Uveitis

Non-infectious posterior uveitis is a form of ocular inflammation that affects the retina and choroid of the eye and leads to blindness. It afflicts approximately 38,000 Americans per year. Patients are usually treated with systemic steroids or corticosteroids therapy, which results in high risks of systemic complications. In 2016, Humira (adalimumab), a human monoclonal antibody that targets tumor necrosis factor-alpha (TNFα), was approved by FDA and became the only systemic noncorticosteroid agent for the treatment of non-infectious uveitis (NIU) and has been widely used since then. In this study, full length or Fab adalimumab antibody in an adeno-associated virus (AAV) vector, such as AAV8.CAG.adalimumab.IgG, or AAV8.CAG.adalimumab.Fab, as well as AAV8.CAG.GFP, will be evaluated for AAV-mediated antibody expression in vivo in mouse ocular tissues via local administration. AAV8.NUL will serve as a control vector. Efficacy studies in rodent EAU model will also be performed to investigate therapeutic potential of AAV-mediated anti-TNFα treatment for NIU.

Vectorized adalimumab sequence have been constructed and tested in vitro. The transduction efficiency and cell type specificity in wild type mouse will further be evaluated. Young adult C57BL/6 and B10.RIII mice (8-10 weeks old) will be used for this study. Vectors including AAV8.CAG.adalimumab.IgG, AAV8.CAG.adalimumab.Fab, AAV8.CAG.GFP and AAV8.NUL will be delivered in mouse eyes via subretinal (SR) injection at different doses (1×10⁷, 1×10⁸ and 1×10⁹ vg/eye) in 1 μl of formulation buffer. Fundus and OCT imaging will be performed at 1, 2 and 4 weeks after SR injection. Ocular samples will be collected at 5 weeks post administration. Levels of antibody or fusion protein expression in ocular tissues will be quantified by ELISA. Cell type specificity will be determined by immunofluorescent staining with various retinal cell markers. Test vector(s) will be selected for efficacy study. Different routes of administration (ROA) including suprachoroidal, intracameral and intravitreal injections will also be explored. The preferred ROA will be used for efficacy study.

Efficacy studies will be conducted by inducing experimental autoimmune uveitis (EAU) in B10.RIII mice by immunization with the human IRBP peptide. T-cell mediated ocular autoimmune response will occur in this model with a peak from approximately 11 to 18 days post-induction. Test vector will be administrated in mouse eye via preferred ROA at 2 weeks before or 1 week after induction of EAU. Contralateral eye will be delivered with AAV.NUL vector and serve as a control. Fundus and OCT imaging, electroretinography (ERG) and Optokinetic nystagmus (OKN) will be tested at 10, 17 and 30 days following induction of EAU to monitor the progress of the disease. Ocular tissues or whole eyeballs will be collected at 5 weeks post EAU induction. Levels of antibody or fusion protein expression in ocular tissues will be detected and quantified by ELISA. Retina structure changes and neuron survival will be evaluated by histology and immunofluorescent staining.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A pharmaceutical composition for treating angioedema including hereditary angioedema in a human subject in need thereof, comprising an AAV vector comprising:
(a) a viral capsid that is at least 95% identical to the amino acid sequence of AAV8 capsid (SEQ ID NO: 143); AAVrh10 capsid (SEQ ID NO: 145); or AAV9 capsid (SEQ ID NO: 144); and
(b) an artificial genome comprising an expression cassette flanked by AAV ITRs wherein the expression cassette comprises a transgene encoding a full-length or substantially full-length anti-kallikrein (anti-pKal) mAb, or an antigen-binding fragment thereof, operably linked to one or more regulatory sequences that control expression of the transgene in human liver cells or human muscle cells;
wherein said AAV vector is formulated for administration to the subject, optionally wherein administration is intravenous, subcutaneous, intranasal, or intramuscular.

2. The pharmaceutical composition of claim 2, wherein the anti-pKal mAb is lanadelumab.

3. A pharmaceutical composition for delivering lanadelumab to the bloodstream to treat hereditary angioedema in a human subject in need thereof, said composition comprising a recombinant AAV comprising a transgene encoding lanadelumab operably linked to one or more regulatory sequences that control expression of the transgene in muscle and/or liver cells, wherein said recombinant AAV is administered to said human subject at a dose sufficient to result in expression from the transgene and secretion of lanadelumab into the bloodstream of the human subject to produce lanadelumab plasma levels of at least 5 μg/ml to at least 35 μg/ml lanadelumab in said subject.

4. The method of claim 3, wherein the lanadelumab plasma levels are 20 μg/ml to 35 μg/ml and the lanadelumab plasma levels are maintained for at least three months.

5. The method of claim 3 or 4 wherein the lanadelumab antibody secreted into the plasma exhibits greater a greater than at least 40%, 45%, 50%, 55%, 60%, 65% or 70 reduction in pKal activity as measured by a kinetic enzymatic functional assay wherein the activity of the lanadelumab antibody is measured at 2 weeks, 3 weeks, 4 weeks, 5 weeks, 6 weeks, 7 weeks, 8 weeks, 9 weeks, 10 weeks, 11 weeks or 12 weeks after said administration.

6. A pharmaceutical composition for delivery of an antibody or antigen binding fragment thereof to the bloodstream of a human subject in need thereof, comprising an AAV vector comprising:
(a) An AAV viral capsid that infects liver and/or muscle cells; and
(b) an artificial genome comprising an expression cassette flanked by AAV ITRs wherein the expression cassette comprises a transgene encoding a full-length antibody or an antigen-binding fragment thereof, operably linked to a chimeric promoter that directs expression in muscle and liver cells;
wherein said AAV vector is formulated for intramuscular administration.

7. The pharmaceutical composition of claim 6 wherein the chimeric promoter is LMTP6 (SEQ ID NO: 320), LMTP13 (SEQ ID NO: 321), LMTP14 (SEQ ID NO: 322), LMTP15 (SEQ ID NO: 323), LMTP18 (SEQ ID NO: 324), LMTP19 (SEQ ID NO: 325), or LMTP20 (SEQ ID NO: 326).

8. A pharmaceutical composition for treating non-infectious uveitis in a human subject in need thereof, comprising an AAV vector comprising:
(a) a viral capsid that is at least 95% identical to the amino acid sequence of an AAV2.7m8 (SEQ ID NO: 142), an AAV8 capsid (SEQ ID NO: 143), or AAV9 capsid (SEQ ID NO: 144); and
(b) an artificial genome comprising an expression cassette flanked by AAV ITRs wherein the expression cassette comprises a transgene encoding a substantially full-length or full-length anti-tumor necrosis factor-alpha (anti-TNFα) mAb or an antigen-binding fragment thereof, a substantially full-length or full-length anti-complement component 5 (C5) mAb or an antigen-binding fragment thereof, a substantially full-length or full-length anti-interleukin-6 (IL-6) mAb or an antigen-binding fragment thereof, or a substantially full-length or full-length anti-interleukin-6 receptor (IL-6R) mAb or an antigen-binding fragment thereof, operably linked to one or more regulatory sequences that control expression of the transgene in human retina cells;
wherein said AAV vector is formulated for subretinal, intravitreal, intranasal, or suprachoroidal administration to the subject.

9. The pharmaceutical composition of claim 8 wherein the anti-TNFα mAb is adalimumab, infliximab or golimumab; the anti-C5 mAb is tesidolumab or ravulizumab; the anti-IL-6 mAb is siltuximab, clazakimzumab, sirukumab, olokizumab or gerilimzumab; or the anti-IL-6R mAb is satralizumab, sarilumab or tocilizumab.

10. A pharmaceutical composition for treating Alzheimer's disease, frontotemporal dementia (FD), tauopathies, progressive supranuclear palsy, chronic traumatic encephalopathy, Pick's Complex, and primary age-related tauopathy, Huntington's disease, juvenile Huntington's disease, Parkinson's disease, synucleinopathies, ALS, migraines, or cluster headaches in a human subject in need thereof, comprising an adeno-associated virus (AAV) vector having:
(a) a viral capsid that is at least 95% identical to the amino acid sequence of an AAV8 capsid (SEQ ID NO: 143), AAV9 capsid (SEQ ID NO: 144), AAVrh10 capsid (SEQ ID NO: 145), an AAVrh20 capsid, an AAVrh39 capsid or an AAVcy5 capsid; and
(b) an artificial genome comprising an expression cassette flanked by AAV inverted terminal repeats (ITRs), wherein the expression cassette comprises a transgene encoding a full-length or substantially full-length anti-amyloid beta (anti-Aβ), anti-sortilin, anti-Tau protein (anti-Tau), anti-semaphorin 4D (anti-SEMA4D), anti-alpha synuclein (anti-SNCA), anti-superoxide dismutase-1 (anti-SOD1) or anti-calcitonin gene-related peptide receptor (anti-CGRPR) mAb, or an antigen-binding fragment thereof, operably linked to one or more regulatory sequences that control expression of the transgene in human CNS, muscle, or liver cells;
wherein said AAV vector is formulated for administration to the subject, optionally wherein administration is intrathecal, intravenous, subcutaneous, intranasal, or intramuscular.

11. The pharmaceutical composition of claim 10, wherein the anti-Aβ mAb is solanezumab, lecanemab, or GSK933776; the anti-sortilin mAb is AL-001; the anti-Tau mAb is ABBV-8E12, UCB-0107, or NI-105 (BIIB076); the anti-SEMA4D mAb is VX15/2503; the anti-SNCA mAb is prasinezumab, NI-202 (BIIB054), or MED-1341; the anti-SOD1 mAb is NI-2041.10D12 or NI-204.12G7; and the anti-CGRPR mAb is eptinezumab, fremanezumab, or galcanezumab.

12. A pharmaceutical composition for treating retinal disorders including diabetic retinopathy, myopic choroidal neovascularization (mCNV), macular degeneration (e.g., neovascular (wet) or dry age-related macular degeneration (nAMD)), macular edema (e.g., macular edema following a retinal vein occlusion (RVO) or diabetic macular edema (DME)), retinal vein occlusion, diabetic retinopathy (DR), non-infectious uveitis, or glaucoma, or abnormal vascularization of the retina in a human subject in need thereof, comprising an AAV vector comprising:
(a) a viral capsid that is at least 95% identical to the amino acid sequence of an AAV2.7m8 capsid (142), an AAV8 capsid (SEQ ID NO: 143), or an AAV9 capsid (SEQ ID NO: 144); and
(b) an artificial genome comprising an expression cassette flanked by AAV ITRs wherein the expression cassette comprises a transgene encoding a full-length or substantially full-length anti-vascular endothelial growth factor (anti-VEGF), anti-erythropoietin receptor (anti-EPOR), anti-Aβ, anti-activin receptor like kinase 1 (anti-ALK1), anti-complement component 5 (anti-C5), anti-endoglin (anti-ENG), anti-complement component 1Q (anti-CC1Q), anti-tumor necrosis factor-alpha (anti-TNFα), or anti-pKal mAb or an antigen-binding fragment thereof, operably linked to one or more regulatory sequences that control expression of the transgene in human retinal cells;
wherein said AAV vector is formulated for subretinal, intravitreal, suprachoroidal, or intranasal administration to said subject.

13. The pharmaceutical composition of claim 12 wherein the anti-VEGF mAb is sevacizumab; anti-EPOR mAb is LKA-651 (NSV2) or LKA-651 (NSV3); anti-Aβ mAb is solanezumab, lecanemab, or GSK933776; anti-ALK1 mAb is ascrinvacumab; anti-C5 mAb is tesidolumab or ravulizumab; anti-ENG mAb is carotuximab; the anti-CC1Q mAb is ANX-007; wherein anti-TNFα mAb is adalimumab, infliximab or golimumab; and the anti-pKal mAb is lanadelumab.

14. A pharmaceutical composition for treating multiple sclerosis in a human subject in need thereof, comprising an AAV vector comprising:
(a) a viral capsid that is at least 95% identical to the amino acid sequence of an AAV8 capsid (SEQ ID NO: 143), AAV9 capsid (SEQ ID NO: 144), AAVrh10 capsid (SEQ ID NO: 145), an AAVrh20 capsid, an AAVrh39 capsid or an AAVcy5 capsid; and
(b) an artificial genome comprising an expression cassette flanked by AAV ITRs wherein the expression cassette comprises a transgene encoding a full-length or substantially full-length anti-repulsive guidance molecule-A (anti-RGMa) mAb, or an antigen-binding fragment thereof, operably linked to one or more regulatory sequences that control expression of the transgene in human CNS, liver, or muscle cells;
wherein said AAV vector is formulated for administration to the subject, optionally wherein administration is intrathecal, intravenous, subcutaneous, intranasal, or intramuscular.

15. The pharmaceutical composition of claim 14, wherein the anti-RGMa mAb is elezanumab.

16. A pharmaceutical composition for treating amyloidosis (ATTR), familial amyloid cardiomyopathy (FAC), or familial amyloid polyneuropathy (FAP) in a human subject in need thereof, comprising AAV vector comprising:
(c) a viral capsid that is at least 95% identical to the amino acid sequence of an AAV8 capsid (SEQ ID NO: 143), an AAV9 capsid (SEQ ID NO: 144), an AAVrh10 capsid (SEQ ID NO: 145), an AAVrh20 capsid, an AAVrh39 capsid, or an AAVcy5 capsid; and
(a) an artificial genome comprising an expression cassette flanked by AAV ITRs wherein the expression cassette comprises a transgene encoding a full-length or substantially full-length anti-TTR mAb, or an antigen-binding fragment thereof, operably linked to one or more regulatory sequences that control expression of the transgene in human liver cells or human muscle cells;
wherein said AAV vector is formulated for administration to the subject, optionally wherein administration is intravenous, subcutaneous, intranasal, or intramuscular.

17. The pharmaceutical composition of claim 16, wherein the anti-TTR mAb is NI-301 or PRX-004.

18. A pharmaceutical composition for treating fibrotic disorders, pulmonary fibrosis, cystic fibrosis (CF), idiopathic pulmonary fibrosis (IPF), liver cirrhosis, atrial fibrosis, endomyocardial fibrosis, old myocardial infarction, arthrofibrosis, Crohn's disease, ulcerative colitis, mediastinal fibrosis, myelofibrosis (MF), nephrogenic systemic fibrosis (NSF), progressive massive fibrosis (PMF), and retroperitoneal fibrosis (RPF) in a human subject in need thereof, comprising an AAV vector comprising:
(c) a viral capsid that is at least 95% identical to the amino acid sequence of an AAV8 capsid (SEQ ID NO: 143), an AAV9 capsid (SEQ ID NO: 144), or AAVrh10 (SEQ ID NO: 145); and
(d) an artificial genome comprising an expression cassette flanked by AAV ITRs wherein the expression cassette comprises a transgene encoding a full-length or substantially full-length anti-connective tissue growth factor (anti-CTGF) mAb, or an antigen-binding fragment thereof, operably linked to one or more regulatory sequences that control expression of the transgene in human liver cells or human muscle cells;
wherein said AAV vector is formulated for administration to said subject, optionally wherein administration is intravenous, subcutaneous, intranasal, or intramuscular.

19. The pharmaceutical composition of claim 18, wherein the anti-CTGF mAb is pamrevlumab.

20. A pharmaceutical composition for treating non-infectious uveitis, neuromyelitis optica (NMO), diabetic retinopathy (DR), or diabetic macular edema (DME) in a human subject in need thereof, comprising an AAV vector comprising:
(a) a viral capsid that is at least 95% identical to the amino acid sequence of an AAV8 capsid (SEQ ID NO: 143), an AAV2.7m8 capsid (SEQ ID NO: 142) or an AAV9 capsid (SEQ ID NO: 144); and
(b) an artificial genome comprising an expression cassette flanked by AAV inverted terminal repeats (ITRs) wherein the expression cassette comprises a transgene encoding a full-length or substantially full-length anti-interleukin-6 receptor (anti-IL6R), anti-interleukin 6 (IL6), or anti-cluster of differentiation 19 (anti-CD19) mAb, or an antigen-binding fragment thereof, operably linked to one or more regulatory sequences that control expression of the transgene in human retinal cells;
wherein said AAV vector is formulated for administration to said subject, optionally wherein administration is intranasal, subretinal, intravitreal, or suprachoroidal.

21. The pharmaceutical composition of claim 20, wherein the anti-IL6R mAb is satralizumab, sarilumab, or tocilizumab, or the anti-IL6 mAb is siltuximab, clazakizumab, sirukumab, olokizumab, or gerilimzumab, or the anti-CD19 mAb is inebilizumab.

22. A pharmaceutical composition for reducing, inhibiting or ameliorating a detrimental immune response in a human subject in need thereof, comprising an AAV vector comprising:
(a) a viral capsid that is at least 95% identical to the amino acid sequence of an AAV8 capsid (SEQ ID NO: 143), an AAV9 capsid (SEQ ID NO: 144), an AAVrh10 capsid (SEQ ID NO: 145); and
(b) an artificial genome comprising an expression cassette flanked by AAV ITRs wherein the expression cassette comprises a transgene encoding a substantially full-length or full-length mAb of an anti-interleukin-6 receptor (anti-IL6R) or anti-interleukin-6 (IL6), or an antigen-binding fragment thereof, operably linked to one or more regulatory sequences that control expression of the transgene in human liver cells or muscle cells;
wherein said AAV vector is formulated for subcutaneous, intramuscular, intravenous or pulmonary administration to the subject.

23. The pharmaceutical composition of claim 22, wherein the anti-IL6R mAb is satralizumab, sarilumab, or tocilizumab, or the anti-IL6 mAb is siltuximab, clazakizumab, sirukumab, olokizumab, or gerilimzumab.

24. A pharmaceutical composition for treating inflammatory bowel disease (IBD) including UC and CD in a human subject in need thereof, comprising an AAV vector comprising:
(a) a viral capsid that is at least 95% identical to the amino acid sequence of AAV8 capsid (SEQ ID NO: 143); AAV9 capsid (SEQ ID NO: 144); or AAVrh10 capsid (SEQ ID NO: 145); and
(b) an artificial genome comprising an expression cassette flanked by AAV ITRs wherein the expression cassette comprises a transgene encoding a full-length or substantially full-length anti-integrin β7 subunit (anti-ITGB7) mAb, or an antigen-binding fragment thereof, operably linked to one or more regulatory sequences that control expression of the transgene in human liver cells or human muscle cells;
wherein said AAV vector is formulated for administration to the subject, optionally wherein administration is intravenous, subcutaneous, intranasal, or intramuscular.

25. The pharmaceutical composition of claim 24, wherein the anti-ITGB7 mAb is etrolizumab.

26. A pharmaceutical composition for treating osteoporosis or abnormal bone loss or weakness (e.g., treating giant cell tumor of bone, treating treatment-induced bone loss, slowing the loss of (or increasing) bone mass in breast and prostate cancer patients, preventing skeletal-related events due to bone metastasis, or for decreasing bone resorption and turnover in a human subject in need thereof, comprising an AAV vector comprising:
(a) a viral capsid that is at least 95% identical to the amino acid sequence of an AAV8 capsid (SEQ ID NO: 143); AAVrh10 capsid (SEQ ID NO: 145); or an AAV9 capsid (SEQ ID NO: 144);
(b) an artificial genome comprising an expression cassette flanked by AAV ITRs wherein the expression cassette comprises a transgene encoding a full-length or substantially full-length anti-sclerostin (anti-SOST) mAb, or an antigen-binding fragment thereof, operably linked to one or more regulatory sequences that control expression of the transgene in human liver cells or human muscle cells;
wherein said AAV vector is formulated for administration to the subject, optionally wherein administration is intravenous, subcutaneous, intranasal, or intramuscular.

27. The pharmaceutical composition of claim 26, wherein the anti-SOST mAb is romosozumab.

28. A pharmaceutical composition for treating atopic dermatitis in a human subject in need thereof, comprising an AAV vector comprising:
(a) a viral capsid that is at least 95% identical to the amino acid sequence of an AAV8 capsid (SEQ ID NO: 143) or AAV9 capsid (SEQ ID NO: 144); and
(b) an artificial genome comprising an expression cassette flanked by AAV ITRs wherein the expression cassette comprises a transgene encoding an anti-IL13 mAb or anti-IL31RA, or an antigen-binding fragment thereof, operably linked to one or more regulatory sequences that control expression of the transgene in human liver cells or human muscle cells;
wherein said AAV vector is formulated for intravenous administration to the liver or muscle of said subject.

29. The pharmaceutical composition of claim 28 wherein the anti-IL13 or the IL31RA is tralokinumab or nemolizumab.

30. A pharmaceutical composition for treating eosinophilic asthma in a human subject in need thereof, comprising an AAV vector comprising:
(a) a viral capsid that is at least 95% identical to the amino acid sequence of an AAV8 capsid (SEQ ID NO: 143) or an AAV9 capsid (SEQ ID NO: 144); and
(b) an artificial genome comprising an expression cassette flanked by AAV ITRs wherein the expression cassette comprises a transgene encoding an anti-IL5R mAb or anti-IgE mAb, or an antigen-binding fragment thereof, operably linked to one or more regulatory sequences that control expression of the transgene in human liver cells or human muscle cells;
wherein said AAV vector is formulated for intravenous administration to the liver or muscle of said subject.

31. The pharmaceutical composition of claim 30 wherein the anti-IL5R or anti-IgE mAb is reslizumab or omalizumab.

32. A pharmaceutical composition for treating asthma or chronic obstructive pulmonary disease (COPD) in a human subject in need thereof, comprising an AAV vector comprising:
(a) a viral capsid that is at least 95% identical to the amino acid sequence of an AAV8 capsid (SEQ ID NO: 143) or an AAV9 capsid (SEQ ID NO: 144); and
(b) an artificial genome comprising an expression cassette flanked by AAV ITRs wherein the expression cassette comprises a transgene encoding an anti-IL5, anti-IL-5R, anti-IgE, or anti-TSLP mAb, or an antigen-binding fragment thereof, operably linked to one or more regulatory sequences that control expression of the transgene in human liver cells or human muscle cells;
wherein said AAV vector is formulated for intravenous administration to the liver or muscle of said subject.

33. The pharmaceutical composition of claim 32 wherein the anti-IL-5, anti-IL5R, anti-IgE, or anti-TSLP mAb is benralizumab, reslizumab, omalizumab, or tezepelumab.

34. A pharmaceutical composition for treating chronic idiopathic urticaria in a human subject in need thereof, comprising an AAV vector comprising:
(a) a viral capsid that is at least 95% identical to the amino acid sequence of an AAV8 capsid (SEQ ID NO:143) or an AAV9 capsid (SEQ ID NO: 144); and
(b) an artificial genome comprising an expression cassette flanked by AAV ITRs wherein the expression cassette comprises a transgene encoding an anti-IgE mAb, or an antigen-binding fragment thereof, operably linked to one or more regulatory sequences that control expression of the transgene in human liver cells or human muscle cells;
wherein said AAV vector is formulated for intravenous administration to the liver or muscle of said subject.

35. The pharmaceutical composition of claim 34, wherein the anti-IgE mAb is omalizumab.

36. A pharmaceutical composition for treating myasthenia gravis in a human subject in need thereof, comprising an AAV vector comprising:
(c) a viral capsid that is at least 95% identical to the amino acid sequence of an AAV8 capsid (SEQ ID NO: 143) or AAV9 capsid (SEQ ID NO: 144); and
(d) an artificial genome comprising an expression cassette flanked by AAV ITRs wherein the expression cassette comprises a transgene encoding an anti-C5 mAb, or an antigen-binding fragment thereof, operably linked to one or more regulatory sequences that control expression of the transgene in human liver cells or human muscle cells;
wherein said AAV vector is formulated for intravenous administration to the liver or muscle of said subject.

37. The pharmaceutical composition of claim 36 wherein the anti-C5 is ravulizumab.

38. A method of determining human anti-pKal antibody activity in a sample, said method comprising
a. Incubating the sample with activated human pKal;
b. Subsequently incubating the sample having been incubated with the activated human pKal with the synthetic substrate Pro-Phe-Arg-AMC
c. Measure the release of AMC over three hours as compared to a control sample.
